(19) United States
(12) Patent Application Publication
(10) Pub. No.: US 2016/0256573 A1
(43) Pub. Date: Sep. 8, 2016

(54) MODIFIED NUCLEIC ACIDS, AND ACUTE CARE USES THEREOF

(71) Applicant: Moderna Therapeutics, Inc., Cambridge, MA (US)

(72) Inventors: Antonin de Fougerolles, Waterloo (BE); Stephane Bancel, Cambridge, MA (US)

(21) Appl. No.: 15/130,064

(22) Filed: Apr. 15, 2016

Related U.S. Application Data
(62) Division of application No. 14/364,406, filed on Jun. 11, 2014, now abandoned.
(60) Provisional application No. 61/570,708, filed on Dec. 14, 2011.

(51) Int. Cl. A61K 48/00; A61K 9/00; A61K 38/18; A61K 38/19

(52) U.S. Cl. CPC A61K 48/0075; A61K 38/19; A61K 9/0014; A61K 38/1891

(57) ABSTRACT

The invention provides compositions and methods for effecting wound healing in a mammal, where the compositions include therapeutic mRNA which incorporate modified nucleosides and nucleotides.

### STATEMENT OF PRIORITY

This application is divisional of U.S. application Ser. No. 14/364,406 filed Jun. 11, 2014, which is a 35 U.S.C. §371 U.S. National Stage Entry of International Application No. PCT/US2012/068732 filed Dec. 10, 2012, which claims the benefit of priority to U.S. Provisional Patent Application No. 61/570,708, filed Dec. 14, 2011, entitled Modified Nucleic Acids, and Acute Care Uses Thereof, the contents of which are incorporated herein by reference in their entirety.

### REFERENCE TO SEQUENCE LISTING

The present application is being filed along with a Sequence Listing in electronic format. The Sequence Listing file, entitled M13USDIV.txt, was created on Apr. 15, 2016 and is 531,911 bytes in size. The information in electronic format of the Sequence Listing is incorporated herein by reference in its entirety.

### BACKGROUND

Naturally occurring RNAs are synthesized from four basic ribonucleotides: ATP, CTP, UTP and GTP, but may contain post-transcriptionally modified nucleotides. Further, approximately one hundred different nucleoside modifications have been identified in RNA (Rozenski, J, Crain, P, and McCloskey, J. (1999). The RNA Modification Database: 1999 update. Nucl Acids Res 27: 196-197). The role of nucleoside modifications on the immuno-stimulatory potential, stability, and on the translation efficiency of RNA, and the consequent benefits to this for enhancing protein expression and producing therapeutics however, is unclear.

There are multiple problems with prior methodologies of effecting protein expression. For example, heterologous deoxyribonucleic acid (DNA) introduced into a cell can be inherited by daughter cells (whether or not the heterologous DNA has integrated into the chromosome) or by offspring. Introduced DNA can integrate into host cell genomic DNA at some frequency, resulting in alterations and/or damage to the host cell genomic DNA. In addition, multiple steps must occur before a protein is made. Once inside the cell, DNA must be transported into the nucleus where it is transcribed into RNA. The RNA transcribed from DNA must then enter the cytoplasm where it is translated into protein. This need for multiple processing steps creates lag times before the generation of a protein of interest. Further, it is difficult to obtain DNA expression in cells; frequently DNA enters cells but is not expressed or not expressed at reasonable rates or concentrations. This can be a particular problem when DNA is introduced into cells such as primary cells or modified cell lines.

There is a need in the art for synthesis of biological modalities to address the modulation of intracellular translation of nucleic acids, and the use of these biological modalities in acute care situations, such as for wound healing after injury, for the treatment of mammalian subjects in need thereof.

### SUMMARY

The present disclosure provides, inter alia, modified nucleosides, modified nucleotides, and modified nucleic acids These modified nucleic acids are capable of being introduced into a target cell or target tissue of a mammalian subject and rapidly translated into a polypeptide of interest, which is particularly useful in acute care situations.

In one embodiment, the present invention provides a synthetic isolated RNA comprising a first region of linked nucleosides encoding a polypeptide of interest, said polypeptide of interest, a first terminal region located at the 5′ terminus of said first region comprising a 5′ untranslated region (UTR), a second terminal region located at the 3′ terminus of said first region comprising a 3′ UTR and a 3′ tailing region of linked nucleosides. The first region, the first terminal region, the second terminal region and/or the 3′ tailing region may comprise at least one modified nucleoside. In one aspect the modified nucleoside is not 5-methylcytosine or pseudouridine. The 5′UTR and/or the 3′UTR of the synthetic isolated RNA may be the native 5′UTR or the native 3′UTR of the encoded polypeptide of interest. The 5′UTR may comprise a translational initiation sequence such as, but not limited to, a Kozak sequence or an internal ribosome entry site (IRES).

In one embodiment, the polypeptide of interest may be selected from, but is not limited to SEQ ID NO: 86-170.

The first terminal region may comprise at least one 5′ cap structure such as, but not limited to, Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azido-guanosine, Cap2 and Cap4.

The 3′ tailing region may include a PolyA tail or a PolyA-G quartet. The PolyA tail may be approximately 150 to 170 nucleotides in length, such as, but not limited to, approximately 160 nucleotides in length.

The synthetic isolated RNA may be purified.

Methods of treating a mammalian subject in need thereof by administering the synthetic isolated RNA comprising at least one 5′ cap structure are also provided. The mammalian subject may be suffering from and/or is at risk of developing an acute or life-threatening disease and/or condition. The mammalian subject may be suffering from a traumatic injury. The mammalian subject may be administered a synthetic isolated RNA comprising a first region encoding a polypeptide of interest which may accelerate wound healing.

In one aspect the present invention provides a method of treating a mammalian subject suffering from or at risk of developing an acute or life-threatening disease or condition, comprising administering to the subject an effective dose of a modified RNA encoding a polypeptide of interest. The polypeptide of interest may be capable of treating or reducing the severity of the disease or condition.

The mammalian subject may be suffering from a bacterial infection. The polypeptide of interest may accelerate recovery from a bacterial infection and/or accelerate resistance to a viral infection. The polypeptide of interest may be a viral antigen or an anti-microbial peptide (AMP) which may comprise lethal activity against a plurality of bacterial pathogens.

The mammalian subject may be suffering from a traumatic injury. The polypeptide of interest may be include, but is not limited to, Platelet Derived Growth Factor (PDGF), Epidermal Growth Factor (EGF), Vascular Endothelial Growth Factor (VEGF), Keratinocyte Growth Factor (KGF), Fibroblast Growth Factor (FGF) and Transforming Growth Factor (TGF).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DETAILED DESCRIPTION

The present disclosure provides, inter alia, generation of modified nucleic acids that exhibit a reduced innate immune response when introduced into a population of cells and use of such modified nucleic acids in acute care situations. In a therapeutic context, the modified nucleic acids are developed very quickly, e.g., in minutes or hours. Any of the approximately 22,000 proteins encoded in the human genome and an infinite number of variants thereof, can be quickly made and administered in vivo using this technology.

In general, exogenous unmodified nucleic acids, particularly viral nucleic acids, introduced into cells induce an innate immune response, resulting in cytokine and interferon (IFN) production and cell death. However, it is of great interest for therapeutics, diagnostics, reagents and for biological assays to deliver a nucleic acid, e.g., a ribonucleic acid (RNA) inside a cell, either in vivo or ex vivo, such as to cause intracellular translation of the nucleic acid and production of the encoded protein. Of particular importance is the delivery and function of a non-integrative nucleic acid, as nucleic acids characterized by integration into a target cell are generally imprecise in their expression levels, deleteriously transferable to progeny and neighbor cells, and suffer from the substantial risk of causing mutation. Provided herein in part are nucleic acids encoding useful polypeptides capable of modulating a cell's function and/or activity, and methods of making and using these nucleic acids and polypeptides. As described herein, these nucleic acids are capable of reducing the innate immune activity of a population of cells into which they are introduced, thus increasing the efficiency of protein production in that cell population. Further, one or more additional advantageous activities and/or properties of the nucleic acids and proteins of the present disclosure are described.

Accordingly, in a first aspect, provided is the use of modified nucleic acids in acute care situations, particularly life-threatening situations such as traumatic injury, or bacterial or viral infections.

In some embodiments, the chemical modifications can be located on the sugar moiety of the nucleotide.

In some embodiments, the chemical modifications can be located on the phosphate backbone of the nucleotide.

### DEFINITIONS

At various places in the present specification, substituents of compounds of the present disclosure are disclosed in groups or in ranges. It is specifically intended that the present disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

About: As used herein, the term “about” means+/−10% of the recited value.

Accelerate: As used herein, the term “accelerate” means to speed up or hasten.

Acute: As used herein, the term “acute” means sudden or severe.

Animal: As used herein, “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions.

Bifunctional: As used herein, the term “bifunctional” refers to any substance, molecule or moiety which is capable of or maintains at least two functions. The functions may effect the same outcome or a different outcome. The structure that produces the function may be the same or different. For example, bifunctional modified RNAs of the present invention may encode a cytotoxic peptide (a first function) while those nucleosides which comprise the encoding RNA are, in and of themselves, cytotoxic (second function). In this example, delivery of the bifunctional modified RNA to a cancer cell would produce not only a peptide or protein molecule which may ameliorate or treat the cancer but would also deliver a cytotoxic payload of nucleosides to the cell should degradation, instead of translation of the modified RNA, occur.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, “biologically active” refers to a characteristic of any substance that has activity in a biological system and/or organism. For instance, a substance that, when administered to an organism, has a biological effect on that organism, is considered to be biologically active. In particular embodiments, where a nucleic acid is biologically active, a portion of that nucleic acid that shares at least one biological activity of the whole nucleic acid is typically referred to as a “biologically active” portion.

Chemical terms: The following provides the definition of various chemical terms from “acyl” to “thiol.”

The term “acyl,” as used herein, represents a hydrogen or an alkyl group (e.g., a haloalkyl group), as defined herein, that is attached to the parent molecular group through a carbonyl group, as defined herein, and is exemplified by formyl (i.e., a carboxyaldehyde group), acetyl, propionyl, butanoyl and the like. Exemplary unsubstituted acyl groups include from 1 to 7, from 1 to 11, or from 1 to 21 carbons. In some embodiments, the alkyl group is further substituted with 1, 2, 3, or 4 substituents as described herein.

The term “acylamino,” as used herein, represents an acyl group, as defined herein, attached to the parent molecular group though an amino group, as defined herein (i.e., —N(R^{N1})—C(O)—R, where R is H or an optionally substituted C₁₋₆, C₁₋₁₀, or C₁₋₂₀ alkyl group and R^{N1} is as defined herein). Exemplary unsubstituted acylamino groups include from 1 to 41 carbons (e.g., from 1 to 7, from 1 to 13, from 1 to 21, from 2 to 7, from 2 to 13, from 2 to 21, or from 2 to 41 carbons). In some embodiments, the alkyl group is further substituted with 1, 2, 3, or 4 substituents as described herein, and/or the amino group is —NH₂ or —NHR^{N1}, wherein R^{N1} is, independently, OH, NO₂, NH₂, NR^{N2}₂, SO₂OR^{N2}, SO₂R^{N2}, SOR^{N2}, alkyl, or aryl, and each R^{N2} can be H, alkyl, or aryl.

The term “acyloxy,” as used herein, represents an acyl group, as defined herein, attached to the parent molecular group though an oxygen atom (i.e., —O—C(O)—R, where R is H or an optionally substituted C₁₋₆, C₁₋₁₀, or C₁₋₂₀ alkyl group). Exemplary unsubstituted acyloxy groups include from 1 to 21 carbons (e.g., from 1 to 7 or from 1 to 11 carbons). In some embodiments, the alkyl group is further substituted with 1, 2, 3, or 4 substituents as described herein, and/or the amino group is —NH₂ or —NHR^{N1}, wherein R^{N1} is, independently, OH, NO₂, NH₂, NR^{N2}₂, SO₂OR^{N2}, SO₂R^{N2}, SOR^{N2}, alkyl, or aryl, and each R^{N2} can be H, alkyl, or aryl.

The term “alkaryl,” as used herein, represents an aryl group, as defined herein, attached to the parent molecular group through an alkylene group, as defined herein. Exemplary unsubstituted alkaryl groups are from 7 to 30 carbons (e.g., from 7 to 16 or from 7 to 20 carbons, such as C₁₋₆ alk-C₆₋₁₀ aryl, C₁₋₁₀ alk-C₆₋₁₀ aryl, or C₁₋₂₀ alk-C₆₋₁₀ aryl). In some embodiments, the alkylene and the aryl each can be further substituted with 1, 2, 3, or 4 substituent groups as defined herein for the respective groups. Other groups preceded by the prefix “alk-” are defined in the same manner, where “alk” refers to a C₁₋₆ alkylene, unless otherwise noted, and the attached chemical structure is as defined herein.

The term “alkcycloalkyl” represents a cycloalkyl group, as defined herein, attached to the parent molecular group through an alkylene group, as defined herein (e.g., an alkylene group of from 1 to 4, from 1 to 6, from 1 to 10, or form 1 to 20 carbons). In some embodiments, the alkylene and the cycloalkyl each can be further substituted with 1, 2, 3, or 4 substituent groups as defined herein for the respective group.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, 2-butenyl, and the like. Alkenyls include both cis and trans isomers. Alkenyl groups may be optionally substituted with 1, 2, 3, or 4 substituent groups that are selected, independently, from amino, aryl, cycloalkyl, or heterocyclyl (e.g., heteroaryl), as defined herein, or any of the exemplary alkyl substituent groups described herein.

The term “alkenyloxy” represents a chemical substituent of formula —OR, where R is a C₂₋₂₀ alkenyl group (e.g., C₂₋₆ or C₂₋₁₀ alkenyl), unless otherwise specified. Exemplary alkenyloxy groups include ethenyloxy, propenyloxy, and the like. In some embodiments, the alkenyl group can be further substituted with 1, 2, 3, or 4 substituent groups as defined herein (e.g., a hydroxy group).

The term “alkheteroaryl” refers to a heteroaryl group, as defined herein, attached to the parent molecular group through an alkylene group, as defined herein. Exemplary unsubstituted alkheteroaryl groups are from 2 to 32 carbons (e.g., from 2 to 22, from 2 to 18, from 2 to 17, from 2 to 16, from 3 to 15, from 2 to 14, from 2 to 13, or from 2 to 12 carbons, such as C₁₋₆ alk-C₁₋₁₂ heteroaryl, C₁₋₁₀ alk-C₁₋₁₂ heteroaryl, or C₁₋₂₀ alk-C₁₋₁₂ heteroaryl). In some embodiments, the alkylene and the heteroaryl each can be further substituted with 1, 2, 3, or 4 substituent groups as defined herein for the respective group. Alkheteroaryl groups are a subset of alkheterocyclyl groups.

The term “alkheterocyclyl” represents a heterocyclyl group, as defined herein, attached to the parent molecular group through an alkylene group, as defined herein. Exemplary unsubstituted alkheterocyclyl groups are from 2 to 32 carbons (e.g., from 2 to 22, from 2 to 18, from 2 to 17, from 2 to 16, from 3 to 15, from 2 to 14, from 2 to 13, or from 2 to 12 carbons, such as C₁₋₆ alk-C₁₋₁₂ heterocyclyl, C₁₋₁₀ alk-C₁₋₁₂ heterocyclyl, or C₁₋₂₀ alk-C₁₋₁₂ heterocyclyl). In some embodiments, the alkylene and the heterocyclyl each can be further substituted with 1, 2, 3, or 4 substituent groups as defined herein for the respective group.

The term “alkoxy” represents a chemical substituent of formula —OR, where R is a C₁₋₂₀ alkyl group (e.g., C₁₋₆ or C₁₋₁₀ alkyl), unless otherwise specified. Exemplary alkoxy groups include methoxy, ethoxy, propoxy (e.g., n-propoxy and isopropoxy), t-butoxy, and the like. In some embodiments, the alkyl group can be further substituted with 1, 2, 3, or 4 substituent groups as defined herein (e.g., hydroxy or alkoxy).

The term “alkoxyalkoxy” represents an alkoxy group that is substituted with an alkoxy group. Exemplary unsubstituted alkoxyalkoxy groups include between 2 to 40 carbons (e.g., from 2 to 12 or from 2 to 20 carbons, such as C₁₋₆ alkoxy-C₁₋₆ alkoxy, C₁₋₁₀ alkoxy-C₁₋₁₀ alkoxy, or C₁₋₂₀ alkoxy-C₁₋₂₀ alkoxy). In some embodiments, the each alkoxy group can be further substituted with 1, 2, 3, or 4 substituent groups as defined herein.

The term “alkoxyalkyl” represents an alkyl group that is substituted with an alkoxy group. Exemplary unsubstituted alkoxyalkyl groups include between 2 to 40 carbons (e.g., from 2 to 12 or from 2 to 20 carbons, such as C₁₋₆ alkoxy-C₁₋₆ alkyl, C₁₋₁₀ alkoxy-C₁₋₁₀ alkyl, or C₁₋₂₀ alkoxy-C₁₋₂₀ alkyl). In some embodiments, the alkyl and the alkoxy each can be further substituted with 1, 2, 3, or 4 substituent groups as defined herein for the respective group.

The term “alkoxycarbonyl,” as used herein, represents an alkoxy, as defined herein, attached to the parent molecular group through a carbonyl atom (e.g., —C(O)—OR, where R is H or an optionally substituted C₁₋₆, C₁₋₁₀, or C₁₋₂₀ alkyl group). Exemplary unsubstituted alkoxycarbonyl include from 1 to 21 carbons (e.g., from 1 to 11 or from 1 to 7 carbons). In some embodiments, the alkoxy group is further substituted with 1, 2, 3, or 4 substituents as described herein.

The term “alkoxycarbonylalkoxy,” as used herein, represents an alkoxy group, as defined herein, that is substituted with an alkoxycarbonyl group, as defined herein (e.g., —O-alkyl-C(O)—OR, where R is an optionally substituted C₁₋₆, C₁₋₁₀, or C₁₋₂₀ alkyl group). Exemplary unsubstituted alkoxycarbonylalkoxy include from 3 to 41 carbons (e.g., from 3 to 10, from 3 to 13, from 3 to 17, from 3 to 21, or from 3 to 31 carbons, such as C₁₋₆ alkoxycarbonyl-C₁₋₆ alkoxy, alkoxycarbonyl-C₁₋₁₀ alkoxy, or C₁₋₂₀ alkoxycarbonyl-C₁₋₂₀ alkoxy). In some embodiments, each alkoxy group is further independently substituted with 1, 2, 3, or 4 substituents, as described herein (e.g., a hydroxy group).

The term “alkoxycarbonylalkyl,” as used herein, represents an alkyl group, as defined herein, that is substituted with an alkoxycarbonyl group, as defined herein (e.g., -alkyl-C(O)—OR, where R is an optionally substituted C₁₋₂₀, C₁₋₁₀, or C₁₋₆ alkyl group). Exemplary unsubstituted alkoxycarbonylalkyl include from 3 to 41 carbons (e.g., from 3 to 10, from 3 to 13, from 3 to 17, from 3 to 21, or from 3 to 31 carbons, such as C₁₋₆ alkoxycarbonyl-C₁₋₆ alkyl, C₁₋₁₀ alkoxycarbonyl-C₁₋₁₀ alkyl, or C₁₋₂₀ alkoxycarbonyl-C₁₋₂₀ alkyl). In some embodiments, each alkyl and alkoxy group is further independently substituted with 1, 2, 3, or 4 substituents as described herein (e.g., a hydroxy group).

The term “alkyl,” as used herein, is inclusive of both straight chain and branched chain saturated groups from 1 to 20 carbons (e.g., from 1 to 10 or from 1 to 6), unless otherwise specified. Alkyl groups are exemplified by methyl, ethyl, n- and iso-propyl, n-, sec-, iso- and tert-butyl, neopentyl, and the like, and may be optionally substituted with one, two, three, or, in the case of alkyl groups of two carbons or more, four substituents independently selected from the group consisting of: (1) C₁₋₆ alkoxy; (2) C₁₋₆ alkylsulfinyl; (3) amino, as defined herein (e.g., unsubstituted amino (i.e., —NH₂) or a substituted amino (i.e., —N(R^{N1})₂, where R^{N1} is as defined for amino); (4) C₆₋₁₀ aryl-C₁₋₆ alkoxy; (5) azido; (6) halo; (7) (C₂₋₉ heterocyclyl)oxy; (8) hydroxy; (9) nitro; (10) oxo (e.g., carboxyaldehyde or acyl); (11) C₁₋₇ spirocyclyl; (12) thioalkoxy; (13) thiol; (14) —CO₂R^{A′}, where R^{A′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alk-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (15) —C(O)NR^{B′}R^{C′}, where each of R^{B′} and R^{C′} is, independently, selected from the group consisting of (a) hydrogen, (b) C₁₋₆ alkyl, (c) C₆₋₁₀ aryl, and (d) C₁₋₆ alk-C₆₋₁₀ aryl; (16) —SO₂R^{D′}, where R^{D′} is selected from the group consisting of (a) C₁₋₆ alkyl, (b) C₆₋₁₀ aryl, (c) C₁₋₆ alk-C₆₋₁₀ aryl, and (d) hydroxy; (17) —SO₂NR^{E′}R^{F′}, where each of R^{E′} and R^{F′} is, independently, selected from the group consisting of (a) hydrogen, (b) C₁₋₆ alkyl, (c) C₆₋₁₀ aryl and (d) C₁₋₆ alk-C₆₋₁₀ aryl; (18) —C(O)R^{G′}, where R^{G′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alk-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (19) —NR^{H′}C(O)R^{I′}, wherein R^{H′} is selected from the group consisting of (a1) hydrogen and (b1) C₁₋₆ alkyl, and R^{I′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alk-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (20) —NR^{J′}C(O)OR^{K′}, wherein R^{J′} is selected from the group consisting of (a1) hydrogen and (b1) C₁₋₆ alkyl, and R^{K′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alk-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; and (21) amidine. In some embodiments, each of these groups can be further substituted as described herein. For example, the alkylene group of a C₁-alkaryl can be further substituted with an oxo group to afford the respective aryloyl substituent.

The term “alkylene” and the prefix “alk-,” as used herein, represent a saturated divalent hydrocarbon group derived from a straight or branched chain saturated hydrocarbon by the removal of two hydrogen atoms, and is exemplified by methylene, ethylene, isopropylene, and the like. The term “C_{x-y} alkylene” and the prefix “C_{x-y} alk-” represent alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁₋₆, C₁₋₁₀, C₂₋₂₀, C₂₋₆, C₂₋₁₀, or C₂₋₂₀ alkylene). In some embodiments, the alkylene can be further substituted with 1, 2, 3, or 4 substituent groups as defined herein for an alkyl group.

The term “alkylsulfinyl,” as used herein, represents an alkyl group attached to the parent molecular group through an —S(O)— group. Exemplary unsubstituted alkylsulfinyl groups are from 1 to 6, from 1 to 10, or from 1 to 20 carbons. In some embodiments, the alkyl group can be further substituted with 1, 2, 3, or 4 substituent groups as defined herein.

The term “alkylsulfinylalkyl,” as used herein, represents an alkyl group, as defined herein, substituted by an alkylsulfinyl group. Exemplary unsubstituted alkylsulfinylalkyl groups are from 2 to 12, from 2 to 20, or from 2 to 40 carbons. In some embodiments, each alkyl group can be further substituted with 1, 2, 3, or 4 substituent groups as defined herein.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, 1-propynyl, and the like. Alkynyl groups may be optionally substituted with 1, 2, 3, or 4 substituent groups that are selected, independently, from aryl, cycloalkyl, or heterocyclyl (e.g., heteroaryl), as defined herein, or any of the exemplary alkyl substituent groups described herein.

The term “alkynyloxy” represents a chemical substituent of formula —OR, where R is a C₂₋₂₀ alkynyl group (e.g., C₂₋₆ or C₂₋₁₀ alkynyl), unless otherwise specified. Exemplary alkynyloxy groups include ethynyloxy, propynyloxy, and the like. In some embodiments, the alkynyl group can be further substituted with 1, 2, 3, or 4 substituent groups as defined herein (e.g., a hydroxy group).

The term “amidine,” as used herein, represents a —C(═NH)NH₂ group.

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, alkaryl, cycloalkyl, alkcycloalkyl, carboxyalkyl, sulfoalkyl, heterocyclyl (e.g., heteroaryl), or alkheterocyclyl (e.g., alkheteroaryl), wherein each of these recited R^{N1} groups can be optionally substituted, as defined herein for each group; or two R^{N1} combine to form a heterocyclyl or an N-protecting group, and wherein each R^{N2} is, independently, H, alkyl, or aryl. The amino groups of the invention can be an unsubstituted amino (i.e., —NH₂) or a substituted amino (i.e., —N(R^{N1})₂). In a preferred embodiment, amino is —NH₂ or —NHR^{N1}, wherein R^{N1} is, independently, OH, NO₂, NH₂, NR^{N2}₂, SO₂OR^{N2}, SO₂R^{N2}, SOR^{N2}, alkyl, carboxyalkyl, sulfoalkyl, or aryl, and each R^{N2} can be H, C₁₋₂₀ alkyl (e.g., C₁₋₆ alkyl), or C₆₋₁₀ aryl.

The term “amino acid,” as described herein, refers to a molecule having a side chain, an amino group, and an acid group (e.g., a carboxy group of —CO₂H or a sulfo group of —SO₃H), wherein the amino acid is attached to the parent molecular group by the side chain, amino group, or acid group (e.g., the side chain). In some embodiments, the amino acid is attached to the parent molecular group by a carbonyl group, where the side chain or amino group is attached to the carbonyl group. Exemplary side chains include an optionally substituted alkyl, aryl, heterocyclyl, alkaryl, alkheterocyclyl, aminoalkyl, carbamoylalkyl, and carboxyalkyl. Exemplary amino acids include alanine, arginine, asparagine, aspartic acid, cysteine, glutamic acid, glutamine, glycine, histidine, hydroxynorvaline, isoleucine, leucine, lysine, methionine, norvaline, ornithine, phenylalanine, proline, pyrrolysine, selenocysteine, serine, taurine, threonine, tryptophan, tyrosine, and valine. Amino acid groups may be optionally substituted with one, two, three, or, in the case of amino acid groups of two carbons or more, four substituents independently selected from the group consisting of: (1) C₁₋₆ alkoxy; (2) C₁₋₆ alkylsulfinyl; (3) amino, as defined herein (e.g., unsubstituted amino (i.e., —NH₂) or a substituted amino (i.e., —N(R^{N1})₂, where R^{N1} is as defined for amino); (4) C₆₋₁₀ aryl-C₁₋₆ alkoxy; (5) azido; (6) halo; (7) (C₂₋₉ heterocyclyl)oxy; (8) hydroxy; (9) nitro; (10) oxo (e.g., carboxyaldehyde or acyl); (11) C₁₋₇ spirocyclyl; (12) thioalkoxy; (13) thiol; (14) —CO₂R^{A′}, where R^{A′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alk-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (15) —C(O)NR^{B′}R^{C′}, where each of R^{B′} and R^{C′} is, independently, selected from the group consisting of (a) hydrogen, (b) C₁₋₆ alkyl, (c) C₆₋₁₀ aryl, and (d) C₁₋₆ alk-C₆₋₁₀ aryl; (16) —SO₂R^{D′}, where R^{D′} is selected from the group consisting of (a) C₁₋₆ alkyl, (b) C₆₋₁₀ aryl, (c) C₁₋₆ alk-C₆₋₁₀ aryl, and (d) hydroxy; (17) —SO₂NR^{E′}R^{F′}, where each of R^{E′} and R^{F′} is, independently, selected from the group consisting of (a) hydrogen, (b) C₁₋₆ alkyl, (c) C₆₋₁₀ aryl and (d) C₁₋₆ alk-C₆₋₁₀ aryl; (18) —C(O)R^{G′}, where R^{G′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alk-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (19) —NR^{H′}C(O)R^{I′}, wherein R^{H′} is selected from the group consisting of (a1) hydrogen and (b1) C₁₋₆ alkyl, and R^{I′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alk-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (20) —NR^{J′}C(O)OR^{K′}, wherein R^{J′} is selected from the group consisting of (a1) hydrogen and (b1) C₁₋₆ alkyl, and R^{K′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alk-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; and (21) amidine. In some embodiments, each of these groups can be further substituted as described herein.

The term “aminoalkoxy,” as used herein, represents an alkoxy group, as defined herein, substituted by an amino group, as defined herein. The alkyl and amino each can be further substituted with 1, 2, 3, or 4 substituent groups as described herein for the respective group (e.g., CO₂R^{A′}, where R^{A′} is selected from the group consisting of (a) C₁₋₆ alkyl, (b) C₆₋₁₀ aryl, (c) hydrogen, and (d) C₁₋₆ alk-C₆₋₁₀ aryl, e.g., carboxy).

The term “aminoalkyl,” as used herein, represents an alkyl group, as defined herein, substituted by an amino group, as defined herein. The alkyl and amino each can be further substituted with 1, 2, 3, or 4 substituent groups as described herein for the respective group (e.g., CO₂R^{A′}, where R^{A′} is selected from the group consisting of (a) C₁₋₆ alkyl, (b) C₆₋₁₀ aryl, (c) hydrogen, and (d) C₁₋₆ alk-C₆₋₁₀ aryl, e.g., carboxy).

The term “aryl,” as used herein, represents a mono-, bicyclic, or multicyclic carbocyclic ring system having one or two aromatic rings and is exemplified by phenyl, naphthyl, 1,2-dihydronaphthyl, 1,2,3,4-tetrahydronaphthyl, anthracenyl, phenanthrenyl, fluorenyl, indanyl, indenyl, and the like, and may be optionally substituted with 1, 2, 3, 4, or 5 substituents independently selected from the group consisting of: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alk-C₆₋₁₀ aryl; (8) azido; (9) C₃₋₈ cycloalkyl; (10) C₁₋₆ alk-C₃₋₈ cycloalkyl; (11) halo; (12) C₁₋₁₂ heterocyclyl (e.g., C₁₋₁₂ heteroaryl); (13) (C₁₋₁₂ heterocyclyl)oxy; (14) hydroxy; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alk-C₆₋₁₀ aryl; (18) —(CH₂)_{q}CONR^{B′}R^{C′}, where q is an integer from zero to four and where R^{B′} and R^{C′} are independently selected from the group consisting of (a) hydrogen, (b) C₁₋₆ alkyl, (c) C₆₋₁₀ aryl, and (d) C₁₋₆ alk-C₆₋₁₀ aryl; (19) —(CH₂)_{q}SO₂R^{D′}, where q is an integer from zero to four and where R^{D′} is selected from the group consisting of (a) alkyl, (b) C₆₋₁₀ aryl, and (c) alk-C₆₋₁₀ aryl; (20) —(CH₂)_{q}SO₂NR^{E′}R^{F′}, where q is an integer from zero to four and where each of R^{E′} and R^{F′} is, independently, selected from the group consisting of (a) hydrogen, (b) C₁₋₆ alkyl, (c) C₆₋₁₀ aryl, and (d) C₁₋₆ alk-C₆₋₁₀ aryl; (21) thiol; (22) C₆₋₁₀ aryloxy; (23) C₃₋₈ cycloalkoxy; (24) C₆₋₁₀ aryl-C₁₋₆ alkoxy; (25) C₁₋₆ alk-C₁₋₁₂ heterocyclyl (e.g., C₁₋₆ alk-C₁₋₁₂ heteroaryl); (26) C₂₋₂₀ alkenyl; and (27) C₂₋₂₀ alkynyl. In some embodiments, each of these groups can be further substituted as described herein. For example, the alkylene group of a C₁-alkaryl or a C₁-alkheterocyclyl can be further substituted with an oxo group to afford the respective aryloyl and (heterocyclyl)oyl substituent group.

The term “arylalkoxy,” as used herein, represents an alkaryl group, as defined herein, attached to the parent molecular group through an oxygen atom. Exemplary unsubstituted alkoxyalkyl groups include from 7 to 30 carbons (e.g., from 7 to 16 or from 7 to 20 carbons, such as C₆₋₁₀ aryl-C₁₋₆ alkoxy, C₆₋₁₀ aryl-C₁₋₁₀ alkoxy, or C₆₋₁₀ aryl-C₁₋₂₀ alkoxy). In some embodiments, the arylalkoxy group can be substituted with 1, 2, 3, or 4 substituents as defined herein

The term “aryloxy” represents a chemical substituent of formula —OR′, where R′ is an aryl group of 6 to 18 carbons, unless otherwise specified. In some embodiments, the aryl group can be substituted with 1, 2, 3, or 4 substituents as defined herein.

The term “aryloyl,” as used herein, represents an aryl group, as defined herein, that is attached to the parent molecular group through a carbonyl group. Exemplary unsubstituted aryloyl groups are of 7 to 11 carbons. In some embodiments, the aryl group can be substituted with 1, 2, 3, or 4 substituents as defined herein.

The term “azido” represents an —N₃ group, which can also be represented as —N═N═N.

The term “bicyclic,” as used herein, refer to a structure having two rings, which may be aromatic or non-aromatic. Bicyclic structures include spirocyclyl groups, as defined herein, and two rings that share one or more bridges, where such bridges can include one atom or a chain including two, three, or more atoms. Exemplary bicyclic groups include a bicyclic carbocyclyl group, where the first and second rings are carbocyclyl groups, as defined herein; a bicyclic aryl groups, where the first and second rings are aryl groups, as defined herein; bicyclic heterocyclyl groups, where the first ring is a heterocyclyl group and the second ring is a carbocyclyl (e.g., aryl) or heterocyclyl (e.g., heteroaryl) group; and bicyclic heteroaryl groups, where the first ring is a heteroaryl group and the second ring is a carbocyclyl (e.g., aryl) or heterocyclyl (e.g., heteroaryl) group. In some embodiments, the bicyclic group can be substituted with 1, 2, 3, or 4 substituents as defined herein for cycloalkyl, heterocyclyl, and aryl groups.

The terms “carbocyclic” and “carbocyclyl,” as used herein, refer to an optionally substituted C₃₋₁₂ monocyclic, bicyclic, or tricyclic structure in which the rings, which may be aromatic or non-aromatic, are formed by carbon atoms. Carbocyclic structures include cycloalkyl, cycloalkenyl, and aryl groups.

The term “carbamoyl,” as used herein, represents —C(O)—N(R^{N1})₂, where the meaning of each R^{N1} is found in the definition of “amino” provided herein.

The term “carbamoylalkyl,” as used herein, represents an alkyl group, as defined herein, substituted by a carbamoyl group, as defined herein. The alkyl group can be further substituted with 1, 2, 3, or 4 substituent groups as described herein.

The term “carbamyl,” as used herein, refers to a carbamate group having the structure —NR^{N1}C(═O)OR or —OC(═O)N(R^{N1})₂, where the meaning of each R^{N1} is found in the definition of “amino” provided herein, and R is alkyl, cycloalkyl, alkcycloalkyl, aryl, alkaryl, heterocyclyl (e.g., heteroaryl), or alkheterocyclyl (e.g., alkheteroaryl), as defined herein.

The term “carbonyl,” as used herein, represents a C(O) group, which can also be represented as C═O.

The term “carboxyaldehyde” represents an acyl group having the structure —CHO.

The term “carboxy,” as used herein, means —CO₂H.

The term “carboxyalkoxy,” as used herein, represents an alkoxy group, as defined herein, substituted by a carboxy group, as defined herein. The alkoxy group can be further substituted with 1, 2, 3, or 4 substituent groups as described herein for the alkyl group.

The term “carboxyalkyl,” as used herein, represents an alkyl group, as defined herein, substituted by a carboxy group, as defined herein. The alkyl group can be further substituted with 1, 2, 3, or 4 substituent groups as described herein.

The term “cyano,” as used herein, represents an —CN group.

The term “cycloalkoxy” represents a chemical substituent of formula —OR, where R is a C₃₋₈ cycloalkyl group, as defined herein, unless otherwise specified. The cycloalkyl group can be further substituted with 1, 2, 3, or 4 substituent groups as described herein. Exemplary unsubstituted cycloalkoxy groups are from 3 to 8 carbons. In some embodiment, the cycloalkyl group can be further substituted with 1, 2, 3, or 4 substituent groups as described herein.

The term “cycloalkyl,” as used herein represents a monovalent saturated or unsaturated non-aromatic cyclic hydrocarbon group from three to eight carbons, unless otherwise specified, and is exemplified by cyclopropyl, cyclobutyl, cyclopentyl, cyclohexyl, cycloheptyl, bicyclo[2.2.1.]heptyl, and the like. When the cycloalkyl group includes one carbon-carbon double bond, the cycloalkyl group can be referred to as a “cycloalkenyl” group. Exemplary cycloalkenyl groups include cyclopentenyl, cyclohexenyl, and the like. The cycloalkyl groups of this invention can be optionally substituted with: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alk-C₆₋₁₀ aryl; (8) azido; (9) C₃₋₈ cycloalkyl; (10) C₁₋₆ alk-C₃₋₈ cycloalkyl; (11) halo; (12) C₁₋₁₂ heterocyclyl (e.g., C₁₋₁₂ heteroaryl); (13) (C₁₋₁₂ heterocyclyl)oxy; (14) hydroxy; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alk-C₆₋₁₀ aryl; (18) —(CH₂)_{q}CONR^{B′}R^{C′}, where q is an integer from zero to four and where R^{B′} and R^{C′} are independently selected from the group consisting of (a) hydrogen, (b) C₆₋₁₀ alkyl, (c) C₆₋₁₀ aryl, and (d) C₁₋₆ alk-C₆₋₁₀ aryl; (19) —(CH₂)_{q}SO₂R^{D′}, where q is an integer from zero to four and where R^{D′} is selected from the group consisting of (a) C₆₋₁₀ alkyl, (b) C₆₋₁₀ aryl, and (c) C₁₋₆ alk-C₆₋₁₀ aryl; (20) —(CH₂)_{q}SO₂NR^{E′}R^{F′}, where q is an integer from zero to four and where each of R^{E′} and R^{F′} is, independently, selected from the group consisting of (a) hydrogen, (b) C₆₋₁₀ alkyl, (c) C₆₋₁₀ aryl, and (d) C₁₋₆ alk-C₆₋₁₀ aryl; (21) thiol; (22) C₆₋₁₀ aryloxy; (23) C₃₋₈ cycloalkoxy; (24) C₆₋₁₀ aryl-C₁₋₆ alkoxy; (25) C₁₋₆ alk-C₁₋₁₂ heterocyclyl (e.g., C₁₋₆ alk-C₁₋₁₂ heteroaryl); (26) oxo; (27) C₂₋₂₀ alkenyl; and (28) C₂₋₂₀ alkynyl. In some embodiments, each of these groups can be further substituted as described herein. For example, the alkylene group of a C₁-alkaryl or a C₁-alkheterocyclyl can be further substituted with an oxo group to afford the respective aryloyl and (heterocyclyl)oyl substituent group.

The term “diastereomer,” as used herein means stereoisomers that are not mirror images of one another and are non-superimposable on one another.

The term “effective amount” of an agent, as used herein, is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “halo,” as used herein, represents a halogen selected from bromine, chlorine, iodine, or fluorine.

The term “haloalkoxy,” as used herein, represents an alkoxy group, as defined herein, substituted by a halogen group (i.e., F, Cl, Br, or I). A haloalkoxy may be substituted with one, two, three, or, in the case of alkyl groups of two carbons or more, four halogens. Haloalkoxy groups include perfluoroalkoxys (e.g., —OCF₃), —OCHF₂, —OCH₂F, —OCCl₃, —OCH₂CH₂Br, —OCH₂CH(CH₂CH₂Br)CH₃, and —OCHICH₃. In some embodiments, the haloalkoxy group can be further substituted with 1, 2, 3, or 4 substituent groups as described herein for alkyl groups.

The term “haloalkyl,” as used herein, represents an alkyl group, as defined herein, substituted by a halogen group (i.e., F, Cl, Br, or I). A haloalkyl may be substituted with one, two, three, or, in the case of alkyl groups of two carbons or more, four halogens. Haloalkyl groups include perfluoroalkyls (e.g., —CF₃), —CHF₂, —CH₂F, —CCl₃, —CH₂CH₂Br, —CH₂CH(CH₂CH₂Br)CH₃, and —CHICH₃. In some embodiments, the haloalkyl group can be further substituted with 1, 2, 3, or 4 substituent groups as described herein for alkyl groups.

The term “heteroalkylene,” as used herein, refers to an alkylene group, as defined herein, in which one or two of the constituent carbon atoms have each been replaced by nitrogen, oxygen, or sulfur. In some embodiments, the heteroalkylene group can be further substituted with 1, 2, 3, or 4 substituent groups as described herein for alkylene groups.

The term “heteroaryl,” as used herein, represents that subset of heterocyclyls, as defined herein, which are aromatic: i.e., they contain 4n+2 pi electrons within the mono- or multicyclic ring system. Exemplary unsubstituted heteroaryl groups are of 1 to 12 (e.g., 1 to 11, 1 to 10, 1 to 9, 2 to 12, 2 to 11, 2 to 10, or 2 to 9) carbons. In some embodiment, the heteroaryl is substituted with 1, 2, 3, or 4 substituents groups as defined for a heterocyclyl group.

The term “heterocyclyl,” as used herein represents a 5-, 6- or 7-membered ring, unless otherwise specified, containing one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocyclyl groups are of 1 to 12 (e.g., 1 to 11, 1 to 10, 1 to 9, 2 to 12, 2 to 11, 2 to 10, or 2 to 9) carbons. The term “heterocyclyl” also represents a heterocyclic compound having a bridged multicyclic structure in which one or more carbons and/or heteroatoms bridges two non-adjacent members of a monocyclic ring, e.g., a quinuclidinyl group. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring, such as indolyl, quinolyl, isoquinolyl, tetrahydroquinolyl, benzofuryl, benzothienyl and the like. Examples of fused heterocyclyls include tropanes and 1,2,3,5,8,8a-hexahydroindolizine. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, indazolyl, quinolyl, isoquinolyl, quinoxalinyl, dihydroquinoxalinyl, quinazolinyl, cinnolinyl, phthalazinyl, benzimidazolyl, benzothiazolyl, benzoxazolyl, benzothiadiazolyl, furyl, thienyl, thiazolidinyl, isothiazolyl, triazolyl, tetrazolyl, oxadiazolyl (e.g., 1,2,3-oxadiazolyl), purinyl, thiadiazolyl (e.g., 1,2,3-thiadiazolyl), tetrahydrofuranyl, dihydrofuranyl, tetrahydrothienyl, dihydrothienyl, dihydroindolyl, dihydroquinolyl, tetrahydroquinolyl, tetrahydroisoquinolyl, dihydroisoquinolyl, pyranyl, dihydropyranyl, dithiazolyl, benzofuranyl, isobenzofuranyl, benzothienyl, and the like, including dihydro and tetrahydro forms thereof, where one or more double bonds are reduced and replaced with hydrogens. Still other exemplary heterocyclyls include: 2,3,4,5-tetrahydro-2-oxo-oxazolyl; 2,3-dihydro-2-oxo-1H-imidazolyl; 2,3,4,5-tetrahydro-5-oxo-1H-pyrazolyl (e.g., 2,3,4,5-tetrahydro-2-phenyl-5-oxo-1H-pyrazolyl); 2,3,4,5-tetrahydro-2,4-dioxo-1H-imidazolyl (e.g., 2,3,4,5-tetrahydro-2,4-dioxo-5-methyl-5-phenyl-1H-imidazolyl); 2,3-dihydro-2-thioxo-1,3,4-oxadiazolyl (e.g., 2,3-dihydro-2-thioxo-5-phenyl-1,3,4-oxadiazolyl); 4,5-dihydro-5-oxo-1H-triazolyl (e.g., 4,5-dihydro-3-methyl-4-amino 5-oxo-1H-triazolyl); 1,2,3,4-tetrahydro-2,4-dioxopyridinyl (e.g., 1,2,3,4-tetrahydro-2,4-dioxo-3,3-diethylpyridinyl); 2,6-dioxo-piperidinyl (e.g., 2,6-dioxo-3-ethyl-3-phenylpiperidinyl); 1,6-dihydro-6-oxopyridiminyl; 1,6-dihydro-4-oxopyrimidinyl (e.g., 2-(methylthio)-1,6-dihydro-4-oxo-5-methylpyrimidin-1-yl); 1,2,3,4-tetrahydro-2,4-dioxopyrimidinyl (e.g., 1,2,3,4-tetrahydro-2,4-dioxo-3-ethylpyrimidinyl); 1,6-dihydro-6-oxo-pyridazinyl (e.g., 1,6-dihydro-6-oxo-3-ethylpyridazinyl); 1,6-dihydro-6-oxo-1,2,4-triazinyl (e.g., 1,6-dihydro-5-isopropyl-6-oxo-1,2,4-triazinyl); 2,3-dihydro-2-oxo-1H-indolyl (e.g., 3,3-dimethyl-2,3-dihydro-2-oxo-1H-indolyl and 2,3-dihydro-2-oxo-3,3′-spiropropane-1H-indol-1-yl); 1,3-dihydro-1-oxo-2H-iso-indolyl; 1,3-dihydro-1,3-dioxo-2H-iso-indolyl; 1H-benzopyrazolyl (e.g., 1-(ethoxycarbonyl)-1H-benzopyrazolyl); 2,3-dihydro-2-oxo-1H-benzimidazolyl (e.g., 3-ethyl-2,3-dihydro-2-oxo-1H-benzimidazolyl); 2,3-dihydro-2-oxo-benzoxazolyl (e.g., 5-chloro-2,3-dihydro-2-oxo-benzoxazolyl); 2,3-dihydro-2-oxo-benzoxazolyl; 2-oxo-2H-benzopyranyl; 1,4-benzodioxanyl; 1,3-benzodioxanyl; 2,3-dihydro-3-oxo,4H-1,3-benzothiazinyl; 3,4-dihydro-4-oxo-3H-quinazolinyl (e.g., 2-methyl-3,4-dihydro-4-oxo-3H-quinazolinyl); 1,2,3,4-tetrahydro-2,4-dioxo-3H-quinazolyl (e.g., 1-ethyl-1,2,3,4-tetrahydro-2,4-dioxo-3H-quinazolyl); 1,2,3,6-tetrahydro-2,6-dioxo-7H-purinyl (e.g., 1,2,3,6-tetrahydro-1,3-dimethyl-2,6-dioxo-7H-purinyl); 1,2,3,6-tetrahydro-2,6-dioxo-1H-purinyl (e.g., 1,2,3,6-tetrahydro-3,7-dimethyl-2,6-dioxo-1H-purinyl); 2-oxobenz[c,d]indolyl; 1,1-dioxo-2H-naphth[1,8-c,d]isothiazolyl; and 1,8-naphthylenedicarboxamido. Additional heterocyclics include 3,3a,4,5,6,6a-hexahydro-pyrrolo[3,4-b]pyrrol-(2H)-yl, and 2,5-diazabicyclo[2.2.1]heptan-2-yl, homopiperazinyl (or diazepanyl), tetrahydropyranyl, dithiazolyl, benzofuranyl, benzothienyl, oxepanyl, thiepanyl, azocanyl, oxecanyl, and thiocanyl. Heterocyclic groups also include groups of the formula

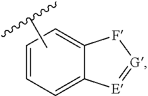

where

E′ is selected from the group consisting of —N— and —CH—; F′ is selected from the group consisting of —N═CH—, —NH—CH₂—, —NH—C(O)—, —NH—, —CH═N—, —CH₂—NH—, —C(O)—NH—, —CH═CH—, —CH₂—, —CH₂CH₂—, —CH₂O—, —OCH₂—, —O—, and —S—; and G′ is selected from the group consisting of —CH— and —N—. Any of the heterocyclyl groups mentioned herein may be optionally substituted with one, two, three, four or five substituents independently selected from the group consisting of: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alk-C₆₋₁₀ aryl; (8) azido; (9) C₃₋₈ cycloalkyl; (10) C₁₋₆ alk-C₃₋₈ cycloalkyl; (11) halo; (12) C₁₋₁₂ heterocyclyl (e.g., C₂₋₁₂ heteroaryl); (13) (C₁₋₁₂ heterocyclyl)oxy; (14) hydroxy; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alk-C₆₋₁₀ aryl; (18) —(CH₂)_{q}CONR^{B′}R^{C′}, where q is an integer from zero to four and where R^{B′} and R^{C′} are independently selected from the group consisting of (a) hydrogen, (b) C₁₋₆ alkyl, (c) C₆₋₁₀ aryl, and (d) C₁₋₆ alk-C₆₋₁₀ aryl; (19) —(CH₂)_{q}SO₂R^{D′}, where q is an integer from zero to four and where R^{D′} is selected from the group consisting of (a) C₁₋₆ alkyl, (b) C₆₋₁₀ aryl, and (c) C₁₋₆ alk-C₆₋₁₀ aryl; (20) —(CH₂)_{q}SO₂NR^{E′}R^{F′}, where q is an integer from zero to four and where each of R^{E′} and R^{F′} is, independently, selected from the group consisting of (a) hydrogen, (b) C₁₋₆ alkyl, (c) C₆₋₁₀ aryl, and (d) C₁₋₆ alk-C₆₋₁₀ aryl; (21) thiol; (22) C₆₋₁₀ aryloxy; (23) C₃₋₈ cycloalkoxy; (24) arylalkoxy; (25) C₁₋₆ alk-C₁₋₁₂ heterocyclyl (e.g., C₁₋₆ alk-C₁₋₁₂ heteroaryl); (26) oxo; (27) (C₁₋₁₂ heterocyclyl)imino; (28) C₂₋₂₀ alkenyl; and (29) C₂₋₂₀ alkynyl. In some embodiments, each of these groups can be further substituted as described herein. For example, the alkylene group of a C₁-alkaryl or a C₁-alkheterocyclyl can be further substituted with an oxo group to afford the respective aryloyl and (heterocyclyl)oyl substituent group.

The term “(heterocyclyl)imino,” as used herein, represents a heterocyclyl group, as defined herein, attached to the parent molecular group through an imino group. In some embodiments, the heterocyclyl group can be substituted with 1, 2, 3, or 4 substituent groups as defined herein.

The term “(heterocyclyl)oxy,” as used herein, represents a heterocyclyl group, as defined herein, attached to the parent molecular group through an oxygen atom. In some embodiments, the heterocyclyl group can be substituted with 1, 2, 3, or 4 substituent groups as defined herein.

The term “(heterocyclyl)oyl,” as used herein, represents a heterocyclyl group, as defined herein, attached to the parent molecular group through a carbonyl group. In some embodiments, the heterocyclyl group can be substituted with 1, 2, 3, or 4 substituent groups as defined herein.

The term “hydrocarbon,” as used herein, represents a group consisting only of carbon and hydrogen atoms.

The term “hydroxy,” as used herein, represents an —OH group.

The term “hydroxyalkenyl,” as used herein, represents an alkenyl group, as defined herein, substituted by one to three hydroxy groups, with the proviso that no more than one hydroxy group may be attached to a single carbon atom of the alkyl group, and is exemplified by dihydroxypropenyl, hydroxyisopentenyl, and the like.

The term “hydroxyalkyl,” as used herein, represents an alkyl group, as defined herein, substituted by one to three hydroxy groups, with the proviso that no more than one hydroxy group may be attached to a single carbon atom of the alkyl group, and is exemplified by hydroxymethyl, dihydroxypropyl, and the like.

The term “isomer,” as used herein, means any tautomer, stereoisomer, enantiomer, or diastereomer of any compound of the invention. It is recognized that the compounds of the invention can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

The term “N-protected amino,” as used herein, refers to an amino group, as defined herein, to which is attached one or two N-protecting groups, as defined herein.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3^{rd} Edition (John Wiley & Sons, New York, 1999), which is incorporated herein by reference. N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, alkaryl groups such as benzyl, triphenylmethyl, benzyloxymethyl, and the like and silyl groups, such as trimethylsilyl, and the like. Preferred N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “oxo” as used herein, represents ═O.

The term “perfluoroalkyl,” as used herein, represents an alkyl group, as defined herein, where each hydrogen radical bound to the alkyl group has been replaced by a fluoride radical. Perfluoroalkyl groups are exemplified by trifluoromethyl, pentafluoroethyl, and the like.

The term “perfluoroalkoxy,” as used herein, represents an alkoxy group, as defined herein, where each hydrogen radical bound to the alkoxy group has been replaced by a fluoride radical. Perfluoroalkoxy groups are exemplified by trifluoromethoxy, pentafluoroethoxy, and the like.

The term “spirocyclyl,” as used herein, represents a C₂₋₇ alkylene diradical, both ends of which are bonded to the same carbon atom of the parent group to form a spirocyclic group, and also a C₁₋₆ heteroalkylene diradical, both ends of which are bonded to the same atom. The heteroalkylene radical forming the spirocyclyl group can containing one, two, three, or four heteroatoms independently selected from the group consisting of nitrogen, oxygen, and sulfur. In some embodiments, the spirocyclyl group includes one to seven carbons, excluding the carbon atom to which the diradical is attached. The spirocyclyl groups of the invention may be optionally substituted with 1, 2, 3, or 4 substituents provided herein as optional substituents for cycloalkyl and/or heterocyclyl groups.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers and/or conformers of the basic molecular structure. Some compounds of the present invention may exist in different tautomeric forms, all of the latter being included within the scope of the present invention.

The term “sulfoalkyl,” as used herein, represents an alkyl group, as defined herein, substituted by a sulfo group of —SO₃H. In some embodiments, the alkyl group can be further substituted with 1, 2, 3, or 4 substituent groups as described herein.

The term “sulfonyl,” as used herein, represents an —S(O)₂— group.

The term “thioalkaryl,” as used herein, represents a chemical substituent of formula —SR, where R is an alkaryl group. In some embodiments, the alkaryl group can be further substituted with 1, 2, 3, or 4 substituent groups as described herein.

The term “thioalkheterocyclyl,” as used herein, represents a chemical substituent of formula —SR, where R is an alkheterocyclyl group. In some embodiments, the alkheterocyclyl group can be further substituted with 1, 2, 3, or 4 substituent groups as described herein.

The term “thioalkoxy,” as used herein, represents a chemical substituent of formula —SR, where R is an alkyl group, as defined herein. In some embodiments, the alkyl group can be further substituted with 1, 2, 3, or 4 substituent groups as described herein.

The term “thiol” represents an —SH group.

Compound: As used herein, the term “compound,” as used herein, is meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide or polypeptide or may apply to a portion, region or feature thereof.

Delivery: As used herein, “delivery” refers to the act or manner of delivering a compound, substance, entity, moiety, cargo or payload.

Delivery Agent: As used herein, “delivery agent” refers to any substance which facilitates, at least in part, the in vivo delivery of a modified nucleic acid to targeted cells.

Device: As used herein, the term “device” means a piece of equipment designed to serve a special purpose. The device may comprise many features such as, but not limited to, components, electrical (e.g., wiring and circuits), storage modules and analysis modules.

Digest: As used herein, the term “digest” means to break apart into smaller pieces or components. When referring to polypeptides or proteins, digestion results in the production of peptides.

Encoded protein cleavage signal: As used herein, “encoded protein cleavage signal” refers to the nucleotide sequence which encodes a protein cleavage signal.

Engineered: As used herein, embodiments of the invention are “engineered” when they are designed to have a feature or property, whether structural or chemical, that varies from a starting point, wild type or native molecule.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least a modified nucleic acid and a delivery agent.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids.

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

Injury: As used herein, the term “injury” results from an act that damages or hurts.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Linker: As used herein, a linker refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker may be of sufficient length as to not interfere with incorporation into a nucleic acid sequence. The linker can be used for any useful purpose, such as to form modified mRNA multimers (e.g., through linkage of two or more modified nucleic acids) or modified mRNA conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

Mobile: As used herein, “mobile” means able to be moved freely or easily.

Modified: As used herein “modified” refers to a changed state or structure of a molecule of the invention. Molecules may be modified in many ways including chemically, structurally, and functionally. In one embodiment, the mRNA molecules of the present invention are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

Module: As used herein, a “module” is an individual self contained unit.

Naturally occurring: As used herein, “naturally occurring” means existing in nature without artificial aid.

Operably linked: As used herein, the phrase “operably linked” refers to a functional connection between two or more molecules, constructs, transcripts, entities, moieties or the like.

Patient: As used herein, “patient” refers to a subject who may seek or be in need of treatment, requires treatment, is receiving treatment, will receive treatment, or a subject who is under care by a trained professional for a particular disease or condition.

Optionally substituted: Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g. alkyl) per se is optional. Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Pharmaceutically acceptable solvate: The term “pharmaceutically acceptable solvate,” as used herein, means a compound of the invention wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prodrug: The present disclosure also includes prodrugs of the compounds described herein. As used herein, “prodrugs” refer to any carriers, typically covalently bonded, which release the active parent drug when administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the present disclosure. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Protein cleavage signal: As used herein “protein cleavage signal” refers to at least one amino acid that flags or marks a polypeptide for cleavage.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Pseudouridine: As used herein, pseudouridine refers to the C-glycoside isomer of the nucleoside uridine. A “pseudouridine analog” is any modification, variant, isoform or derivative of pseudouridine. For example, pseudouridine analogs include but are not limited to 1-carboxymethyl-pseudouridine, 1-propynyl-pseudouridine, 1-taurinomethyl-pseudouridine, 1-taurinomethyl-4-thio-pseudouridine, 1-methyl-pseudouridine (m¹ψ), 1-methyl-4-thio-pseudouridine (m¹s⁴ψ) 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 2-methoxyuridine, 2-methoxy-4-thio-uridine, 4-methoxy-pseudouridine, 4-methoxy-2-thio-pseudouridine, N1-methyl-pseudouridine, 1-methyl-3-(3-amino-3-carboxypropyl)pseudouridine (acp³ψ), and 2′-O-methyl-pseudouridine (ψm).

Purified: As used herein, “purify,” “purified,” “purification” means to make substantially pure or clear from unwanted components, material defilement, admixture or imperfection.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Single unit dose: As used herein, a “single unit dose” is a dose of any therapeutic administered in one dose/at one time/single route/single point of contact, i.e., single administration event.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules of the present invention may be chemical or enzymatic.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Therapeutically effective outcome: As used herein, “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to a disease, disorder, and/or condition, to treat, improve symptoms of, diagnose, prevent, and/or delay the onset of the disease, disorder, and/or condition.

Total daily dose: As used herein, a “total daily dose” is an amount given or prescribed in 24 hr period. It may be administered as a single unit dose.

Transcription factor: As used herein, “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules.

Traumatic: As used herein, the term “traumatic” or “trauma” refers to an injury.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Wound: As used herein, the term “wound” refers to an injury causing damage to a subject. The damage may be the breaking of a membrane such as the skin or damage to underlying tissue.

### Acute Delivery and Use of Modified Nucleic Acids

### Encoded Polypeptides

The modified nucleic acids of the present invention may be designed to encode polypeptides of interest selected from any of several target categories including, but not limited to, wound healing, anti-bacterial and anti-viral.

In one embodiment modified nucleic acids may encode variant polypeptides which have a certain identity with a reference polypeptide sequence. As used herein, a “reference polypeptide sequence” refers to a starting polypeptide sequence. Reference sequences may be wild type sequences or any sequence to which reference is made in the design of another sequence. A “reference polypeptide sequence” may, e.g., be any one of SEQ ID NOs: 86-170 as disclosed herein, e.g., any of SEQ ID NOs 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170.

The term “identity” as known in the art, refers to a relationship between the sequences of two or more peptides, as determined by comparing the sequences. In the art, identity also means the degree of sequence relatedness between peptides, as determined by the number of matches between strings of two or more amino acid residues. Identity measures the percent of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

In some embodiments, the polypeptide variant may have the same or a similar activity as the reference polypeptide. Alternatively, the variant may have an altered activity (e.g., increased or decreased) relative to a reference polypeptide. Generally, variants of a particular modified nucleic acid or polypeptide of the invention will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference modified nucleic acid or polypeptide as determined by sequence alignment programs and parameters described herein and known to those skilled in the art. Such tools for alignment include those of the BLAST suite (Stephen F. Altschul, Thomas L. Madden, Alejandro A. Schäffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.) Other tools are described herein, specifically in the definition of “Identity.”

Default parameters in the BLAST algorithm include, for example, an expect threshold of 10, Word size of 28, Match/Mismatch Scores 1, -2, Gap costs Linear. Any filter can be applied as well as a selection for species specific repeats, e.g., Homo sapiens.

### Wound Healing.

The invention provides for the delivery of wound healing therapeutics to a mammalian subject in need thereof. Proteins are required to facilitate all the key steps in the process of wound healing, including (i) inflammation, (ii) cell motility, (iii) regrowth of cells, and (iv) rebuilding of tissue architecture, such as the epidermis and reconstructing damaged blood vessels in the case of a skin injury. Inappropriate or abnormal protein and gene expression is associated with impaired wound healing or excessive scarring, indicating the importance of the key steps in the wound healing process. Conversely, localized over-expression of proteins and genes has been shown to improve the rate of wound healing in animal models. Thus, high levels of proteins found at the site of a wound indicate key markers that can be regulated using the modified RNA technology in accordance with the invention to increase an immune response and enhance wound healing.

At the onset of an injury, neutrophils are found in abundance at the site of a wound. Neutrophils are cells that express and release cytokines into the circulation or directly into the tissue during an immune response and amplify inflammatory reactions. The released cytokines interact with receptors on targeted immune cells by binding to them, an interaction that triggers specific responses by the targeted cells. There are several different kinds of cytokines found in mammalian subjects, including but not limited to (i) cytokines for stimulating the production of blood cells, (ii) cytokines that function in growth and differentiation as growth factor proteins and (iii) cytokines specialized for immunoregulatory and proinflammatory functions. Specific examples of cytokines include but are not limited to: Platelet Derived Growth Factor (PDGF), Epidermal Growth Factor (EGF), Vascular Endothelial Growth Factor (VEGF), Keratinocyte Growth Factor (KGF), Fibroblast Growth Factor (FGF), and Transforming Growth Factor (TGF). Administration of modified RNA encoding for a specific cytokine in a mammalian subject can increase the cytokine response and improve wound healing, in accordance with the invention.

Macrophages are also present during the inflammation step of wound healing. Macrophages are cells that function by expressing proteins that engulf and digest cellular debris and pathogens. Specific examples of proteins expressed by macrophages include but are not limited to: Cluster of Differentiation Proteins (mCD14), (sCD14), (CD11b), and (CD-68), EGF-like Module-Containing Mucin-like Hormone Receptor-like 1 proteins expressed by the EMR1 gene (EMR1), Macrophage-1 Antigens (MAC-1), and Granulocyte-Macrophage Colony-Stimulating Factor (GM-CSF). GM-CSF, for instance, is a cytokine secreted by macrophages that functions to increase the white blood cell count of a mammalian subject. Monocytes are an example of white blood cells increased by GM-CSF. Monocytes play a critical role in wound healing by (i) replenishing macrophages and dendritic cells and (ii) moving quickly in response to inflammation signals to divide into macrophages and dendritic cells to elicit an immune response. Regulation of GM-CSF through modified RNA delivery to a subject can thereby result in an increase in white blood cell count and a faster and improved immune response.

In response to cytokines and growth factors, Signal Transducer and Activator of Transcription 3 (STAT3) proteins are formed. STAT3 mediates the expression of a variety of genes in response to cell stimuli, resulting in the STAT3 gene and STAT3 proteins having an important role in many cellular processes such as cell growth. Manipulation of the STAT3 gene through modified RNA delivery can enhance important steps of cell regrowth and cell rebuilding.

In a next step of wound healing, proliferation, which is characterized by cell motility and cell regrowth, fibroblasts are predominant and in charge of synthesizing a new extracellular matrix and collagen. Fibroblasts grow and form a new provisional extracellular matrix by excreting collagen and fibronectin, while at the same time epithelial cells form on top of a wound, providing a cover for new tissue to grow. In the step of proliferation, tissue repair markers are found, including but not limited to Cysteine, Protease and Collagen Modifying Enzymes including but not limited to Pro-Collagen-Lysine, 2-Oxoglutarate 5-Dioxygenase and Integrin B5. Regulation of regrowth factors through modified RNA in accordance with the invention can further stimulate improved wound repair and coverage by increasing fibroblast cell secretions.

Finally, in a last step of rebuilding of tissue architecture, a new extracellular matrix is formed and the angiogenesis process of building new capillaries occurs. At this step the technology in accordance with the invention can be used to target genes of interest for amplification or inhibition and for protein-therapy to manipulate angiogenic growth factors including but not limited to Fibroblast Growth Factor (FGF-1) and Vascular Endothelial Growth Factor (VEGF) to improve matrix and vessel formation.

The rapid and timely synthesis and delivery of modified RNAs encoding for protein proteins needed to facilitate wound healing, such as cytokines and, growth factors, is particularly useful in the immediate treatment and care of wound healing, e.g., following a motor vehicle accident, or in military operations such as on the battlefield.

In one embodiment, the modified RNA such as, but not limited to, wound healing therapeutics described herein, may be encapsulated into a lipid nanoparticle or a rapidly eliminating lipid nanoparticle and/or the may be encapsulated into a polymer, hydrogel and/or surgical sealant described herein and/or known in the art. In another embodiment, the modified RNA may be encapsulated into a lipid nanoparticle or a rapidly eliminating lipid nanoparticle prior to being encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.). The modified RNA and/or modified RNA lipid nanoparitice may be encapsulated in any polymer or hydrogel known in the art which may form a gel when injected into a subject.

### Target Selection

According to the present invention, the modified nucleic acids comprise at least a first region of linked nucleosides encoding at least one polypeptide of interest. Non-limiting examples of the polypeptides of interest or “Targets” of the present invention are listed in Table 1. Shown in Table 1, in addition to the description of the gene encoding the polypeptide of interest are the National Center for Biotechnology Information (NCBI) nucleotide reference ID (NM Ref) and the NCBI peptide reference ID (NP Ref). For any particular gene there may exist one or more variants or isoforms. Where these exist, they are shown in the table as well. It will be appreciated by those of skill in the art that disclosed in the Table are potential flanking regions. These are encoded in each nucleotide sequence either to the 5′ (upstream) or 3′ (downstream) of the open reading frame. The open reading frame is definitively and specifically disclosed by teaching the nucleotide reference sequence. Consequently, the sequences taught flanking that encoding the protein are considered flanking regions. It is also possible to further characterize the 5′ and 3′ flanking regions by utilizing one or more available databases or algorithms. Databases have annotated the features contained in the flanking regions of the NCBI sequences and these are available in the art.

TABLE 1
Targets

| | | | SEQ | | SEQ ID |
|---|---|---|---|---|---|
| Target | Description | NM Ref. | ID NO | NP Ref. | NO |
| 1 | Homo sapiens platelet-derived | NM_002607.5 | 1 | NP_002598.4 | 86 |
| | growth factor alpha polypeptide | | | | |
| | (PDGFA), transcript variant 1, | | | | |
| | mRNA | | | | |
| 2 | Homo sapiens platelet-derived | NM_033023.4 | 2 | NP_148983.1 | 87 |
| | growth factor alpha polypeptide | | | | |
| | (PDGFA), transcript variant 2, | | | | |
| | mRNA | | | | |
| 3 | Homo sapiens platelet-derived | NM_002608.2 | 3 | NP_002599.1 | 88 |
| | growth factor beta polypeptide | | | | |
| | (PDGFB), transcript variant 1, | | | | |
| | mRNA | | | | |
| 4 | Homo sapiens platelet-derived | NM_033016.2 | 4 | NP_148937.1 | 89 |
| | growth factor beta polypeptide | | | | |
| | (PDGFB), transcript variant 2, | | | | |
| | mRNA | | | | |
| 5 | Homo sapiens platelet derived | NM_016205.2 | 5 | NP_057289.1 | 90 |
| | growth factor C (PDGFC), transcript | | | | |
| | variant 1, mRNA | | | | |
| 6 | Homo sapiens platelet derived | NM_025208.4 | 6 | NP_079484.1 | 91 |
| | growth factor D (PDGFD), transcript | | | | |
| | variant 1, mRNA | | | | |
| 7 | Homo sapiens platelet derived | NM_033135.3 | 7 | NP_149126.1 | 92 |
| | growth factor D (PDGFD), transcript | | | | |
| | variant 2, mRNA | | | | |
| 8 | Homo sapiens epidermal growth | NM_001963.4 | 8 | NP_001954.2 | 93 |
| | factor (EGF), transcript variant 1, | | | | |
| | mRNA | | | | |
| 9 | Homo sapiens epidermal growth | NM_001178130.1 | 9 | NP_001171601.1 | 94 |
| | factor (EGF), transcript variant 2, | | | | |
| | mRNA | | | | |
| 10 | Homo sapiens epidermal growth | NM_001178131.1 | 10 | NP_001171602.1 | 95 |
| | factor (EGF), transcript variant 3, | | | | |
| | mRNA | | | | |
| 11 | Homo sapiens vascular endothelial | NM_001171623.1 | 11 | NP_001165094.1 | 96 |
| | growth factor A (VEGFA), transcript | | | | |
| | variant 1, mRNA | | | | |
| 12 | Homo sapiens vascular endothelial | NM_001025366.2 | 12 | NP_001020537.2 | 97 |
| | growth factor A (VEGFA), transcript | | | | |
| | variant 1, mRNA | | | | |
| 13 | Homo sapiens vascular endothelial | NM_001171624.1 | 13 | NP_001165095.1 | 98 |
| | growth factor A (VEGFA), transcript | | | | |
| | variant 2, mRNA | | | | |
| 14 | Homo sapiens vascular endothelial | NM_003376.5 | 14 | NP_003367.4 | 99 |
| | growth factor A (VEGFA), transcript | | | | |
| | variant 2, mRNA | | | | |
| 15 | Homo sapiens vascular endothelial | NM_001171625.1 | 15 | NP_001165096.1 | 100 |
| | growth factor A (VEGFA), transcript | | | | |
| | variant 3, mRNA | | | | |
| 16 | Homo sapiens vascular endothelial | NM_001025367.2 | 16 | NP_001020538.2 | 101 |
| | growth factor A (VEGFA), transcript | | | | |
| | variant 3, mRNA | | | | |
| 17 | Homo sapiens vascular endothelial | NM_001171626.1 | 17 | NP_001165097.1 | 102 |
| | growth factor A (VEGFA), transcript | | | | |
| | variant 4, mRNA | | | | |
| 18 | Homo sapiens vascular endothelial | NM_001025368.2 | 18 | NP_001020539.2 | 103 |
| | growth factor A (VEGFA), transcript | | | | |
| | variant 4, mRNA | | | | |
| 19 | Homo sapiens vascular endothelial | NM_001171627.1 | 19 | NP_001165098.1 | 104 |
| | growth factor A (VEGFA), transcript | | | | |
| | variant 5, mRNA | | | | |
| 20 | Homo sapiens vascular endothelial | NM_001025369.2 | 20 | NP_001020540.2 | 105 |
| | growth factor A (VEGFA), transcript | | | | |
| | variant 5, mRNA | | | | |
| 21 | Homo sapiens vascular endothelial | NM_001171628.1 | 21 | NP_001165099.1 | 106 |
| | growth factor A (VEGFA), transcript | | | | |
| | variant 6, mRNA | | | | |
| 22 | Homo sapiens vascular endothelial | NM_001025370.2 | 22 | NP_001020541.2 | 107 |
| | growth factor A (VEGFA), transcript | | | | |
| | variant 6, mRNA | | | | |
| 23 | Homo sapiens vascular endothelial | NM_001171629.1 | 23 | NP_001165100.1 | 108 |
| | growth factor A (VEGFA), transcript | | | | |
| | variant 7, mRNA | | | | |
| 24 | Homo sapiens vascular endothelial | NM_001033756.2 | 24 | NP_001028928.1 | 109 |
| | growth factor A (VEGFA), transcript | | | | |
| | variant 7, mRNA | | | | |
| 25 | Homo sapiens vascular endothelial | NM_001171630.1 | 25 | NP_001165101.1 | 110 |
| | growth factor A (VEGFA), transcript | | | | |
| | variant 8, mRNA | | | | |
| 26 | Homo sapiens vascular endothelial | NM_001171622.1 | 26 | NP_001165093.1 | 111 |
| | growth factor A (VEGFA), transcript | | | | |
| | variant 8, mRNA | | | | |
| 27 | Homo sapiens vascular endothelial | NM_001204385.1 | 27 | NP_001191314.1 | 112 |
| | growth factor A (VEGFA), transcript | | | | |
| | variant 9, mRNA | | | | |
| 28 | Homo sapiens vascular endothelial | NM_001204385.1 | 28 | NP_001191314.1 | 113 |
| | growth factor A (VEGFA), transcript | | | | |
| | variant 9, mRNA | | | | |
| 29 | Homo sapiens vascular endothelial | NM_001204384.1 | 29 | NP_001191313.1 | 114 |
| | growth factor A (VEGFA), transcript | | | | |
| | variant 9, mRNA | | | | |
| 30 | Homo sapiens vascular endothelial | NM_001243733.1 | 30 | NP_001230662.1 | 115 |
| | growth factor B (VEGFB), transcript | | | | |
| | variant VEGFB-167, mRNA | | | | |
| 31 | Homo sapiens vascular endothelial | NM_005429.2 | 31 | NP_005420.1 | 116 |
| | growth factor C (VEGFC), mRNA | | | | |
| 32 | Homo sapiens vascular endothelial | NM_003377.4 | 32 | NP_003368.1 | 117 |
| | growth factor B (VEGFB), transcript | | | | |
| | variant VEGFB-186, mRNA | | | | |
| 33 | Homo sapiens fibroblast growth | NM_002009.3 | 33 | NP_002000.1 | 118 |
| | factor 7 (FGF7), mRNA | | | | |
| 34 | Homo sapiens transforming growth | NM_003236.3 | 34 | NP_003227.1 | 119 |
| | factor, alpha (TGFA), transcript | | | | |
| | variant 1, mRNA | | | | |
| 35 | Homo sapiens transforming growth | NM_001099691.2 | 35 | NP_001093161.1 | 120 |
| | factor, alpha (TGFA), transcript | | | | |
| | variant 2, mRNA | | | | |
| 36 | Homo sapiens transforming growth | NM_000660.4 | 36 | NP_000651.3 | 121 |
| | factor, beta 1 (TGFB1), mRNA | | | | |
| 37 | Homo sapiens transforming growth | NM_001135599.2 | 37 | NP_001129071.1 | 122 |
| | factor, beta 2 (TGFB2), transcript | | | | |
| | variant 1, mRNA | | | | |
| 38 | Homo sapiens transforming growth | NM_003238.3 | 38 | NP_003229.1 | 123 |
| | factor, beta 2 (TGFB2), transcript | | | | |
| | variant 2, mRNA | | | | |
| 39 | Homo sapiens transforming growth | NM_003239.2 | 39 | NP_003230.1 | 124 |
| | factor, beta 3 (TGFB3), mRNA | | | | |
| 40 | Homo sapiens fibroblast growth | NM_000800.4 | 40 | NP_000791.1 | 125 |
| | factor 1 (acidic) (FGF1), transcript | | | | |
| | variant 1, mRNA | | | | |
| 41 | Homo sapiens fibroblast growth | NM_033136.3 | 41 | NP_149127.1 | 126 |
| | factor 1 (acidic) (FGF1), transcript | | | | |
| | variant 2, mRNA | | | | |
| 42 | Homo sapiens fibroblast growth | NM_033137.2 | 42 | NP_149128.1 | 127 |
| | factor 1 (acidic) (FGF1), transcript | | | | |
| | variant 3, mRNA | | | | |
| 43 | Homo sapiens fibroblast growth | NM_001144892.2 | 43 | NP_001138364.1 | 128 |
| | factor 1 (acidic) (FGF1), transcript | | | | |
| | variant 4, mRNA | | | | |
| 44 | Homo sapiens fibroblast growth | NM_001144934.1 | 44 | NP_001138406.1 | 129 |
| | factor 1 (acidic) (FGF1), transcript | | | | |
| | variant 5, mRNA | | | | |
| 45 | Homo sapiens fibroblast growth | NM_001144935.1 | 45 | NP_001138407.1 | 130 |
| | factor 1 (acidic) (FGF1), transcript | | | | |
| | variant 6, mRNA | | | | |
| 46 | Homo sapiens fibroblast growth | NM_001257205.1 | 46 | NP_001244134.1 | 131 |
| | factor 1 (acidic) (FGF1), transcript | | | | |
| | variant 7, mRNA | | | | |
| 47 | Homo sapiens fibroblast growth | NM_001257206.1 | 47 | NP_001244135.1 | 132 |
| | factor 1 (acidic) (FGF1), transcript | | | | |
| | variant 8, mRNA | | | | |
| 48 | Homo sapiens fibroblast growth | NM_001257207.1 | 48 | NP_001244136.1 | 133 |
| | factor 1 (acidic) (FGF1), transcript | | | | |
| | variant 9, mRNA | | | | |
| 49 | Homo sapiens fibroblast growth | NM_001257208.1 | 49 | NP_001244137 | 134 |
| | factor 1 (acidic) (FGF1), transcript | | | | |
| | variant 10, mRNA | | | | |
| 50 | Homo sapiens fibroblast growth | NM_001257209.1 | 50 | NP_001244138.1 | 135 |
| | factor 1 (acidic) (FGF1), transcript | | | | |
| | variant 11, mRNA | | | | |
| 51 | Homo sapiens fibroblast growth | NM_001257210.1 | 51 | NP_001244139.1 | 136 |
| | factor 1 (acidic) (FGF1), transcript | | | | |
| | variant 12, mRNA | | | | |
| 52 | Homo sapiens fibroblast growth | NM_001257211.1 | 52 | NP_001244140.1 | 137 |
| | factor 1 (acidic) (FGF1), transcript | | | | |
| | variant 13, mRNA | | | | |
| 53 | Homo sapiens fibroblast growth | NM_001257212.1 | 53 | NP_001244141.1 | 138 |
| | factor 1 (acidic) (FGF1), transcript | | | | |
| | variant 14, mRNA | | | | |
| 54 | Homo sapiens fibroblast growth | NM_002006.4 | 54 | NP_001997.5 | 139 |
| | factor 2 (basic) (FGF2), mRNA | | | | |
| 55 | Homo sapiens fibroblast growth | NM_005247.2 | 55 | NP_005238.1 | 140 |
| | factor 3 (FGF3), mRNA | | | | |
| 56 | Homo sapiens fibroblast growth | NM_002007.2 | 56 | NP_001998.1 | 141 |
| | factor 4 (FGF4), mRNA | | | | |
| 57 | Homo sapiens fibroblast growth | NM_004464.3 | 57 | NP_004455.2 | 142 |
| | factor 5 (FGF5), transcript variant 1, | | | | |
| | mRNA | | | | |
| 58 | Homo sapiens fibroblast growth | NM_033143.2 | 58 | NP_149134.1 | 143 |
| | factor 5 (FGF5), transcript variant 2, | | | | |
| | mRNA | | | | |
| 59 | Homo sapiens fibroblast growth | NM_020996.1 | 59 | NP_066276.2 | 144 |
| | factor 6 (FGF6), mRNA | | | | |
| 60 | Homo sapiens fibroblast growth | NM_033165.3 | 60 | NP_149355.1 | 145 |
| | factor 8 (androgen-induced) (FGF8), | | | | |
| | transcript variant A, mRNA | | | | |
| 61 | Homo sapiens fibroblast growth | NM_006119.4 | 61 | NP_006110.1 | 146 |
| | factor 8 (androgen-induced) (FGF8), | | | | |
| | transcript variant B, mRNA | | | | |
| 62 | Homo sapiens fibroblast growth | NM_033164.3 | 62 | NP_149354.1 | 147 |
| | factor 8 (androgen-induced) (FGF8), | | | | |
| | transcript variant E, mRNA | | | | |
| 63 | Homo sapiens fibroblast growth | NM_033163.3 | 63 | NP_149353.1 | 148 |
| | factor 8 (androgen-induced) (FGF8), | | | | |
| | transcript variant F, mRNA | | | | |
| 64 | Homo sapiens fibroblast growth | NM_001206389.1 | 64 | NP_001193318.1 | 149 |
| | factor 8 (androgen-induced) (FGF8), | | | | |
| | transcript variant G, mRNA | | | | |
| 65 | Homo sapiens fibroblast growth | NM_002010.2 | 65 | NP_002001.1 | 150 |
| | factor 9 (glia-activating factor) | | | | |
| | (FGF9), mRNA | | | | |
| 66 | Homo sapiens fibroblast growth | NM_004465.1 | 66 | NP_004456 | 151 |
| | factor 10 (FGF10), mRNA | | | | |
| 67 | Homo sapiens fibroblast growth | NM_004112.2 | 67 | NP_004103.1 | 152 |
| | factor 11 (FGF11), mRNA | | | | |
| 68 | Homo sapiens fibroblast growth | NM_021032.4 | 68 | NP_066360.1 | 153 |
| | factor 12 (FGF12), transcript variant | | | | |
| | 1, mRNA | | | | |
| 69 | Homo sapiens fibroblast growth | NM_004113.5 | 69 | NP_004104.3 | 154 |
| | factor 12 (FGF12), transcript variant | | | | |
| | 2, mRNA | | | | |
| 70 | Homo sapiens fibroblast growth | NM_004114.3 | 70 | NP_004105.1 | 155 |
| | factor 13 (FGF13), transcript variant | | | | |
| | 1, mRNA | | | | |
| 71 | Homo sapiens fibroblast growth | NM_001139500.1 | 71 | NP_001132972.1 | 156 |
| | factor 13 (FGF13), transcript variant | | | | |
| | 2, mRNA | | | | |
| 72 | Homo sapiens fibroblast growth | NM_001139501.1 | 72 | NP_001132973.1 | 157 |
| | factor 13 (FGF13), transcript variant | | | | |
| | 3, mRNA | | | | |
| 73 | Homo sapiens fibroblast growth | NM_001139498.1 | 73 | NP_001132970.1 | 158 |
| | factor 13 (FGF13), transcript variant | | | | |
| | 4, mRNA | | | | |
| 74 | Homo sapiens fibroblast growth | NM_001139502.1 | 74 | NP_001132974.1 | 159 |
| | factor 13 (FGF13), transcript variant | | | | |
| | 5, mRNA | | | | |
| 75 | Homo sapiens fibroblast growth | NM_033642.2 | 75 | NP_378668.1 | 160 |
| | factor 13 (FGF13), transcript variant | | | | |
| | 6, mRNA | | | | |
| 76 | Homo sapiens fibroblast growth | NM_004115.3 | 76 | NP_004106.1 | 161 |
| | factor 14 (FGF14), transcript variant | | | | |
| | 1, mRNA | | | | |
| 77 | Homo sapiens fibroblast growth | NM_175929.2 | 77 | NP_787125.1 | 162 |
| | factor 14 (FGF14), transcript variant | | | | |
| | 2, mRNA | | | | |
| 78 | Homo sapiens fibroblast growth | NM_003868.1 | 78 | NP_003859.1 | 163 |
| | factor 16 (FGF16), mRNA | | | | |
| 79 | Homo sapiens fibroblast growth | NM_003867.2 | 79 | NP_003858.1 | 164 |
| | factor 17 (FGF17), mRNA | | | | |
| 80 | Homo sapiens fibroblast growth | NM_003862.2 | 80 | NP_003853.1 | 165 |
| | factor 18 (FGF18), mRNA | | | | |
| 81 | Homo sapiens fibroblast growth | NM_005117.2 | 81 | NP_005108.1 | 166 |
| | factor 19 (FGF19), mRNA | | | | |
| 82 | Homo sapiens fibroblast growth | NM_019851.2 | 82 | NP_062825.1 | 167 |
| | factor 20 (FGF20), mRNA | | | | |
| 83 | Homo sapiens fibroblast growth | NM_019113.2 | 83 | NP_061986.1 | 168 |
| | factor 21 (FGF21), mRNA | | | | |
| 84 | Homo sapiens fibroblast growth | NM_020637.1 | 84 | NP_065688.1 | 169 |
| | factor 22 (FGF22), mRNA | | | | |
| 85 | Homo sapiens fibroblast growth | NM_020638.2 | 85 | NP_065689.1 | 170 |
| | factor 23 (FGF23), mRNA | | | | |

### Anti-Bacterials.

Despite numerous successes in anti-microbial development over the past century, the emergence of resistance worldwide continues to spur the search for novel anti-infectives to replace and/or supplement conventional antibiotics. One area of antimicrobial drug research that shows significant promise is in the discovery and development of anti-microbial peptides (AMPs). To avoid opportunistic infections, animals and humans have evolved a large number of AMPs that can form pores in the cytoplasmic membrane of microorganisms. To date, more than 1700 endogenous AMPs have been isolated, with many being expressed in tissues with direct contact with microorganisms, such as epithelial cells of the skin and the respiratory and digestive systems. AMPs can also be expressed and active systemically through expression in blood.

AMPs are typically small (less than 10 kDa, 15 to 45 amino acid residues), cationic and amphipathic peptides of variable length, sequence and structure with broad spectrum killing activity against a wide range of microorganisms including gram-positive and gram-negative bacteria, enveloped viruses, fungi and some protozoa. AMPs exert their effect by binding to the negatively charged phospholipid bilayer of prokaryotic cells, leading to membrane pore formation and cell lysis. The lack of specific receptors makes it difficult for bacteria to develop resistance to AMPs as they would need to alter the properties of their whole membrane rather than specific receptors. Importantly, eukaryotic cell membranes are generally unaffected by AMPs given their different membrane composition and overall neutrally charged phospholipid bilayers. However, despite promising results in early-stage and even late-stage clinical trials, the unfavorable pharmacokinetics (low bioavailability and protease stability) and high cost of producing these naturally occurring anti-microbial peptides represent a major barrier to their use as anti-microbials in vivo. The modified RNAs provided herein are useful and novel anti-microbial drugs, and are suited to overcome some of the limitations with administration of polypeptide AMPs.

### Anti-Virals.

Viral subunit vaccines consisting of protein target antigens stimulate the immune system to attack invading pathogens. Virus specific protein targets are identified and cultured in cells for mass production and purification as a vaccine. The modified RNAs of the invention are useful to rapidly prime an individual's immune system to respond to emerging viral threats. Once the genomic sequence or antigenic protein of the offending virus is identified, a modified RNA vaccine is generated for immediate administration, without cell culturing or protein manufacture. The subject (e.g., a soldier, government employee or hospital patient exposed or at risk of being exposed to a virus) is treated with a modified RNA vaccine encoding the viral antigen. The antigen is quickly synthesized in the body in a biologically relevant manner and triggers a less broadly immunogenic response, but instead directly primes an immediate response to the specific threat. This approach provides a rapid prophylactic treatment response to new and emerging threats, with minimal side effects where quality and speed are of the essence.

### Modified Nucleosides and Nucleotides

The present invention also includes the building blocks, e.g., modified ribonucleosides, modified ribonucleotides, of the nucleic acids or modified RNA, e.g., modified RNA (or mRNA) molecules. For example, these building blocks can be useful for preparing the nucleic acids or modified RNA of the invention.

In some embodiments, the building block molecule has Formula (IIIa) or (IIIa-1):

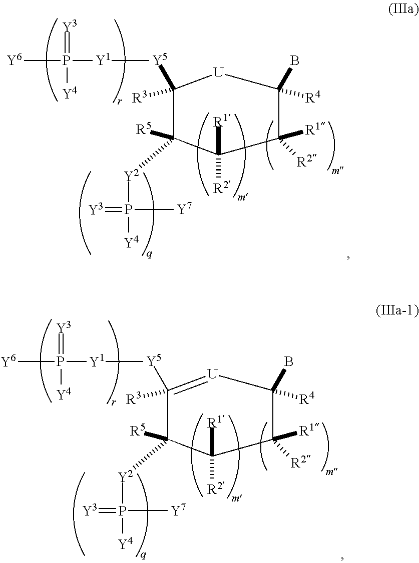

or a pharmaceutically acceptable salt or stereoisomer thereof, wherein the substituents are as described herein (e.g., for Formula (Ia) and (Ia-1)), and wherein when B is an unmodified nucleobase selected from cytosine, guanine, uracil and adenine, then at least one of Y¹, Y², or Y³ is not O.

In some embodiments, the building block molecule, which may be incorporated into a nucleic acids or modified RNA, has Formula (IVa)-(IVb):

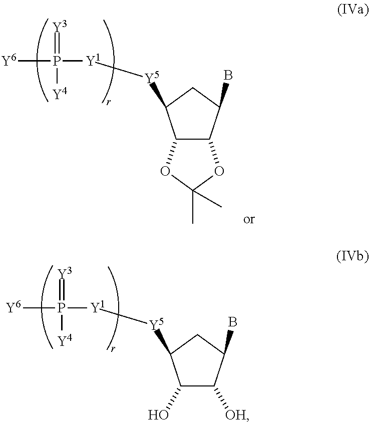

or a pharmaceutically acceptable salt or stereoisomer thereof, wherein B is as described herein (e.g., any one of (b1)-(b43)).

In particular embodiments, Formula (IVa) or (IVb) is combined with a modified uracil (e.g., any one of formulas (b1)-(b9), (b21)-(b23), and (b28)-(b31), such as formula (b1), (b8), (b28), (b29), or (b30)). In particular embodiments, Formula (IVa) or (IVb) is combined with a modified cytosine (e.g., any one of formulas (b10)-(b14), (b24), (b25), and (b32)-(b36), such as formula (b10) or (b32)). In particular embodiments, Formula (IVa) or (IVb) is combined with a modified guanine (e.g., any one of formulas (b15)-(b17) and (b37)-(b40)). In particular embodiments, Formula (IVa) or (IVb) is combined with a modified adenine (e.g., any one of formulas (b18)-(b20) and (b41)-(b43)).

In some embodiments, the building block molecule, which may be incorporated into a nucleic acids or modified RNA, has Formula (IVc)-(IVk):

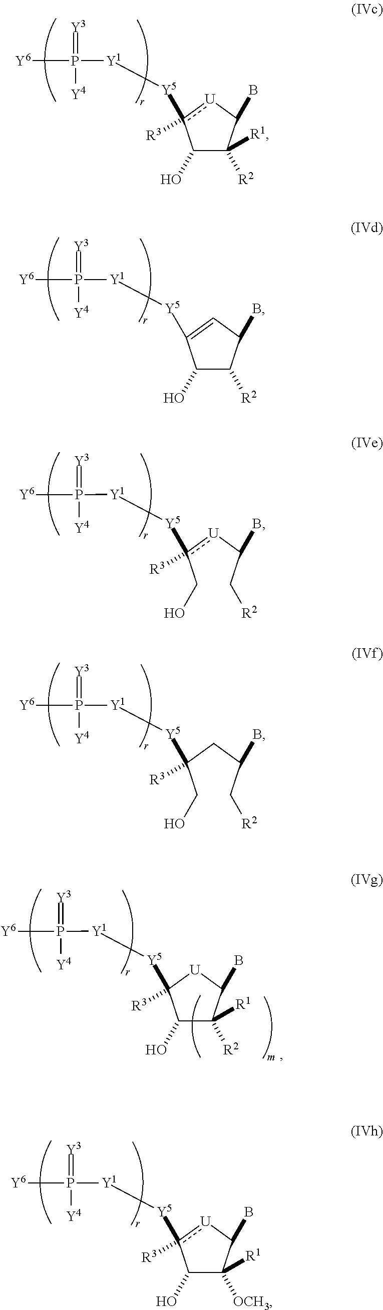

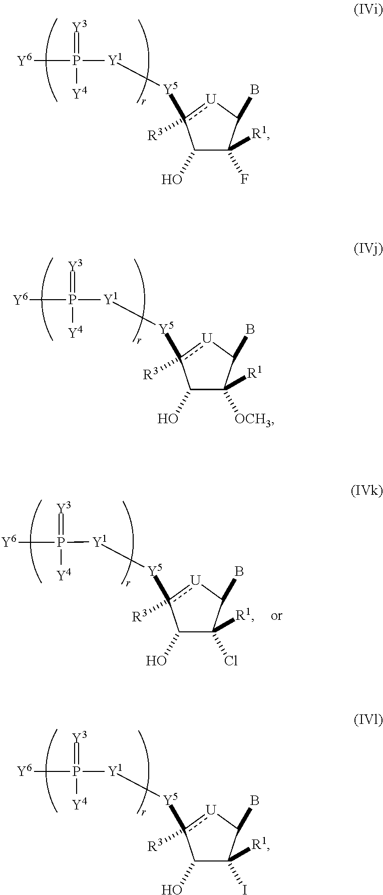

or a pharmaceutically acceptable salt or stereoisomer thereof, wherein B is as described herein (e.g., any one of (b1)-(b43)).

In particular embodiments, one of Formulas (IVc)-(IVk) is combined with a modified uracil (e.g., any one of formulas (b1)-(b9), (b21)-(b23), and (b28)-(b31), such as formula (b1), (b8), (b28), (b29), or (b30)).

In particular embodiments, one of Formulas (IVc)-(IVk) is combined with a modified cytosine (e.g., any one of formulas (b10)-(b14), (b24), (b25), and (b32)-(b36), such as formula (b10) or (b32)).

In particular embodiments, one of Formulas (IVc)-(IVk) is combined with a modified guanine (e.g., any one of formulas (b15)-(b17) and (b37)-(b40)).

In particular embodiments, one of Formulas (IVc)-(IVk) is combined with a modified adenine (e.g., any one of formulas (b18)-(b20) and (b41)-(b43)).

In other embodiments, the building block molecule, which may be incorporated into a nucleic acids or modified RNA has Formula (Va) or (Vb):

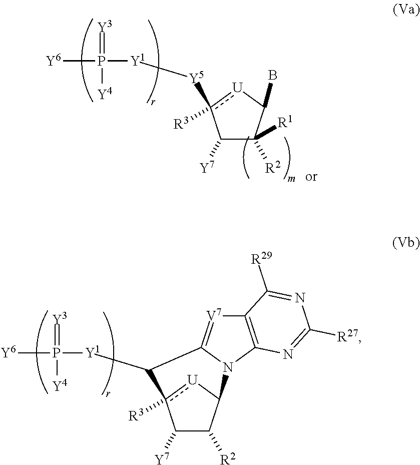

or a pharmaceutically acceptable salt or stereoisomer thereof, wherein B is as described herein (e.g., any one of (b1)-(b43)).

In other embodiments, the building block molecule, which may be incorporated into a nucleic acids or modified RNA has Formula (IXa)-(IXd):

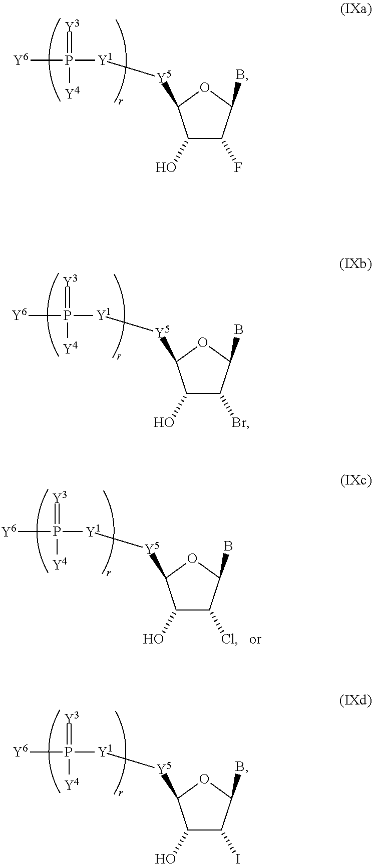

or a pharmaceutically acceptable salt or stereoisomer thereof, wherein B is as described herein (e.g., any one of (b1)-(b43)).
In particular embodiments, one of Formulas (IXa)-(IXd) is combined with a modified uracil (e.g., any one of formulas (b1)-(b9), (b21)-(b23), and (b28)-(b31), such as formula (b1), (b8), (b28), (b29), or (b30)). In particular embodiments, one of Formulas (IXa)-(IXd) is combined with a modified cytosine (e.g., any one of formulas (b10)-(b14), (b24), (b25), and (b32)-(b36), such as formula (b10) or (b32)).
In particular embodiments, one of Formulas (IXa)-(IXd) is combined with a modified guanine (e.g., any one of formulas (b15)-(b17) and (b37)-(b40)).
In particular embodiments, one of Formulas (IXa)-(IXd) is combined with a modified adenine (e.g., any one of formulas (b18)-(b20) and (b41)-(b43)).

In other embodiments, the building block molecule, which may be incorporated into a nucleic acids or modified RNA has Formula (IXe)-(IXg):

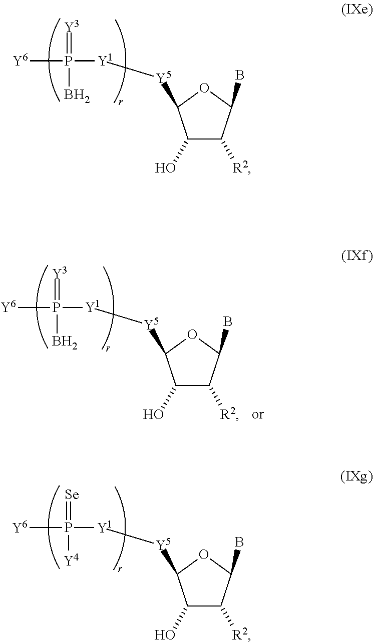

or a pharmaceutically acceptable salt or stereoisomer thereof, wherein B is as described herein (e.g., any one of (b1)-(b43)).

In particular embodiments, one of Formulas (IXe)-(IXg) is combined with a modified uracil (e.g., any one of formulas (b1)-(b9), (b21)-(b23), and (b28)-(b31), such as formula (b1), (b8), (b28), (b29), or (b30)).

In particular embodiments, one of Formulas (IXe)-(IXg) is combined with a modified cytosine (e.g., any one of formulas (b10)-(b14), (b24), (b25), and (b32)-(b36), such as formula (b10) or (b32)).

In particular embodiments, one of Formulas (IXe)-(IXg) is combined with a modified guanine (e.g., any one of formulas (b15)-(b17) and (b37)-(b40)).

In particular embodiments, one of Formulas (IXe)-(IXg) is combined with a modified adenine (e.g., any one of formulas (b18)-(b20) and (b41)-(b43)).

In other embodiments, the building block molecule, which may be incorporated into a nucleic acids or modified RNA has Formula (IXh)-(IXk):

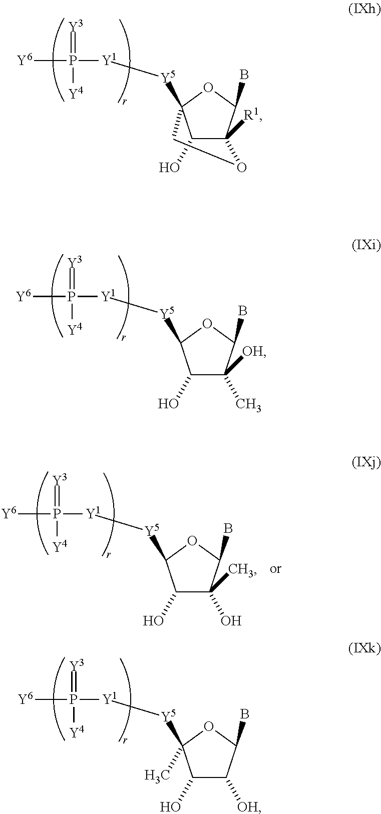

or a pharmaceutically acceptable salt or stereoisomer thereof, wherein B is as described herein (e.g., any one of (b1)-(b43)). In particular embodiments, one of Formulas (IXh)-(IXk) is combined with a modified uracil (e.g., any one of formulas (b1)-(b9), (b21)-(b23), and (b28)-(b31), such as formula (b1), (b8), (b28), (b29), or (b30)). In particular embodiments, one of Formulas (IXh)-(IXk) is combined with a modified cytosine (e.g., any one of formulas (b10)-(b14), (b24), (b25), and (b32)-(b36), such as formula (b10) or (b32)).

In particular embodiments, one of Formulas (IXh)-(IXk) is combined with a modified guanine (e.g., any one of formulas (b15)-(b17) and (b37)-(b40)). In particular embodiments, one of Formulas (IXh)-(IXk) is combined with a modified adenine (e.g., any one of formulas (b18)-(b20) and (b41)-(b43)).

In other embodiments, the building block molecule, which may be incorporated into a nucleic acids or modified RNA has Formula (IXl)-(IXr):

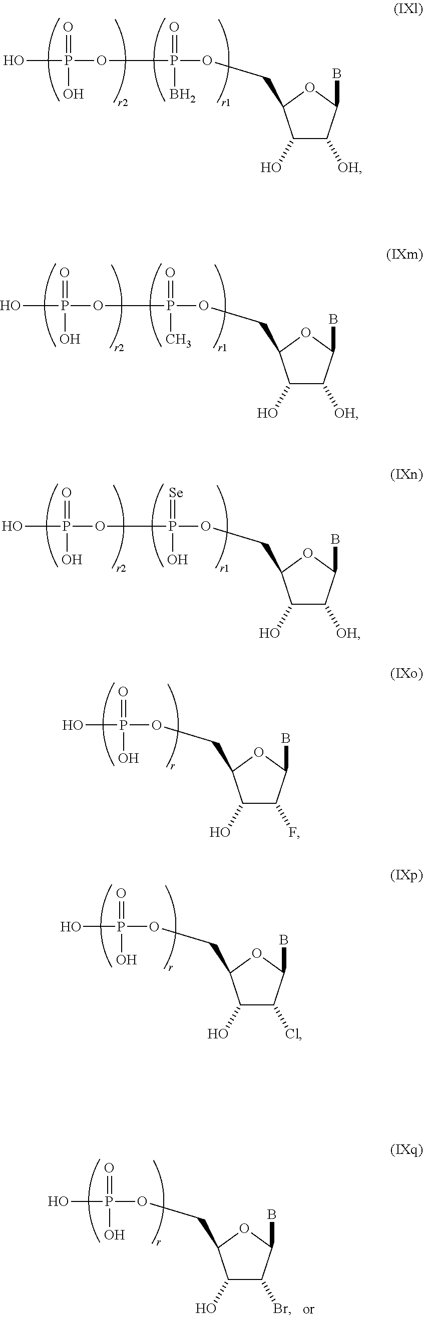

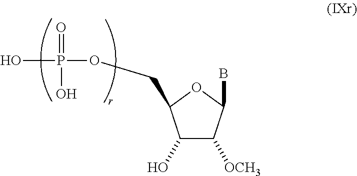

or a pharmaceutically acceptable salt or stereoisomer thereof, wherein each r1 and r2 is, independently, an integer from 0 to 5 (e.g., from 0 to 3, from 1 to 3, or from 1 to 5) and B is as described herein (e.g., any one of (b1)-(b43)).

In particular embodiments, one of Formulas (IXl)-(IXr) is combined with a modified uracil (e.g., any one of formulas (b1)-(b9), (b21)-(b23), and (b28)-(b31), such as formula (b1), (b8), (b28), (b29), or (b30)).

In particular embodiments, one of Formulas (IXl)-(IXr) is combined with a modified cytosine (e.g., any one of formulas (b10)-(b14), (b24), (b25), and (b32)-(b36), such as formula (b10) or (b32)).

In particular embodiments, one of Formulas (IXl)-(IXr) is combined with a modified guanine (e.g., any one of formulas (b15)-(b17) and (b37)-(b40)). In particular embodiments, one of Formulas (IXl)-(IXr) is combined with a modified adenine (e.g., any one of formulas (b18)-(b20) and (b41)-(b43)).

In some embodiments, the building block molecule, which may be incorporated into a nucleic acids or modified RNA can be selected from the group consisting of:

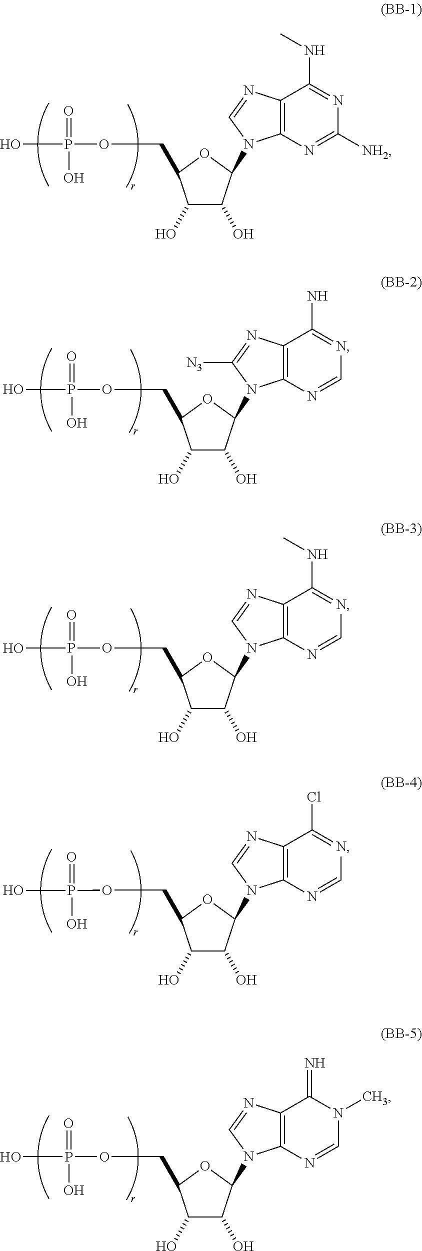

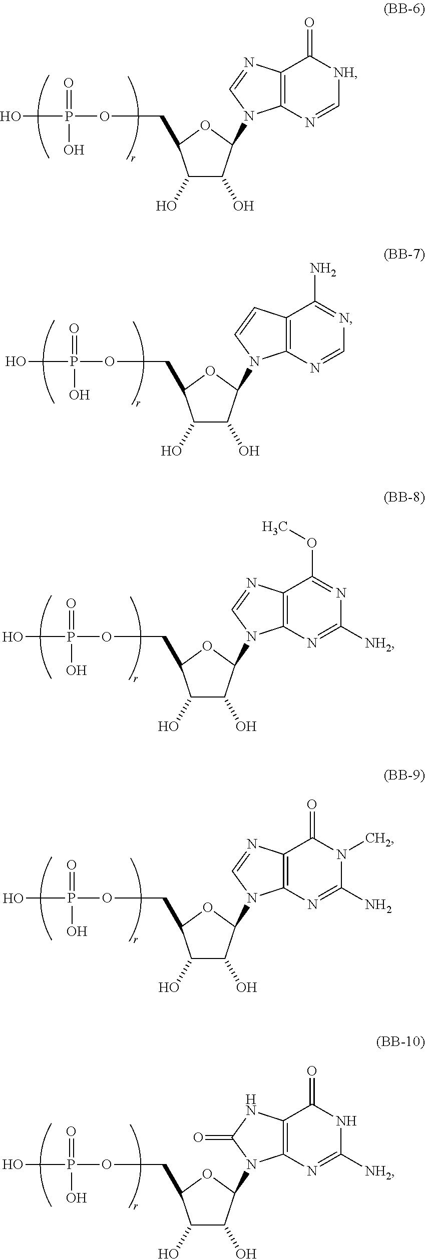

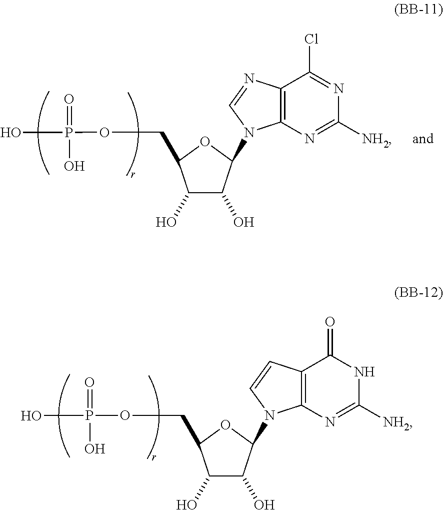

or a pharmaceutically acceptable salt or stereoisomer thereof, wherein each r is, independently, an integer from 0 to 5 (e.g., from 0 to 3, from 1 to 3, or from 1 to 5).

In some embodiments, the building block molecule, which may be incorporated into a nucleic acids or modified RNA can be selected from the group consisting of:

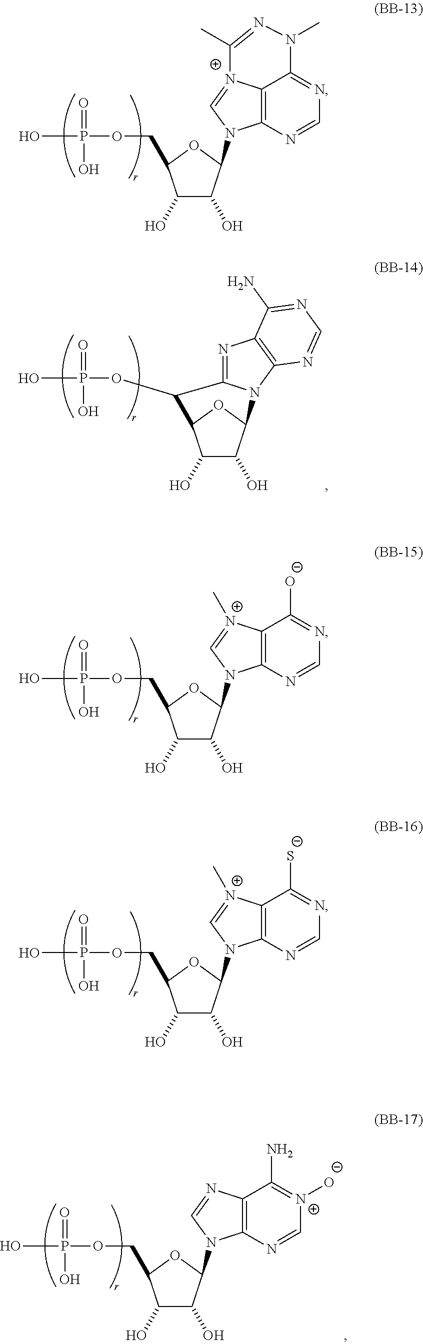

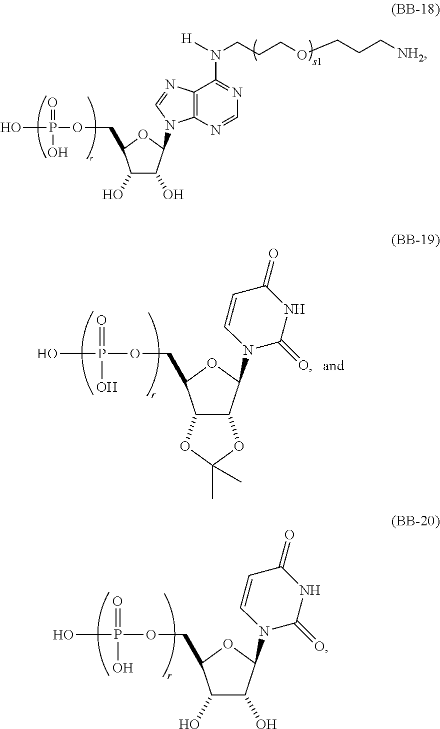

or a pharmaceutically acceptable salt or stereoisomer thereof, wherein each r is, independently, an integer from 0 to 5 (e.g., from 0 to 3, from 1 to 3, or from 1 to 5) and s1 is as described herein.

In some embodiments, the building block molecule, which may be incorporated into a nucleic acid (e.g., RNA, mRNA, or modified RNA), is a modified uridine (e.g., selected from the group consisting of:

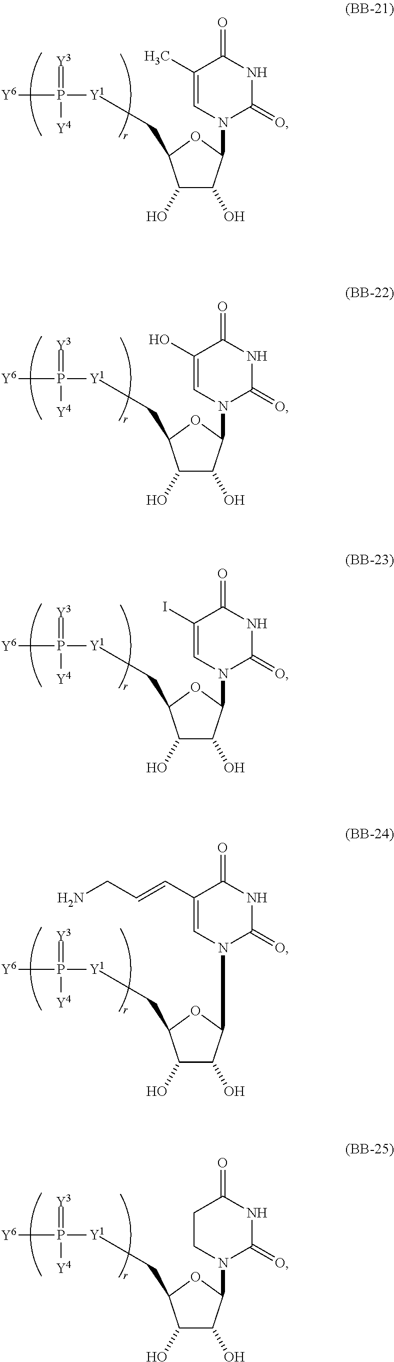

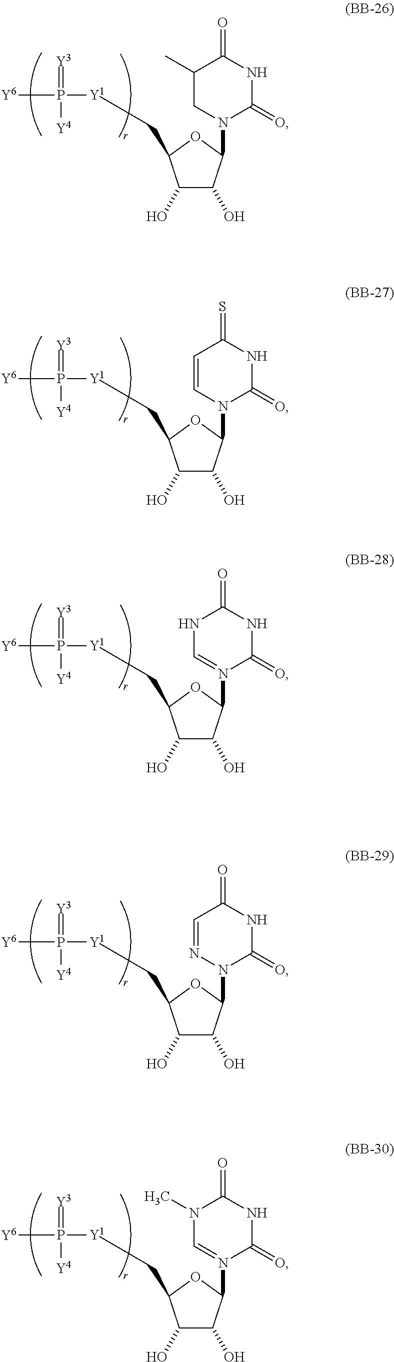

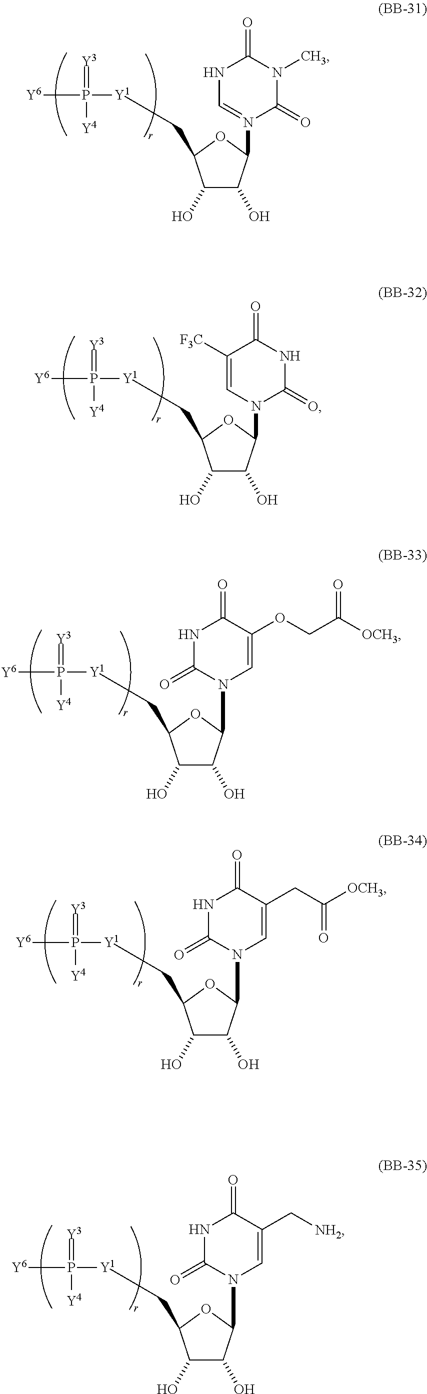

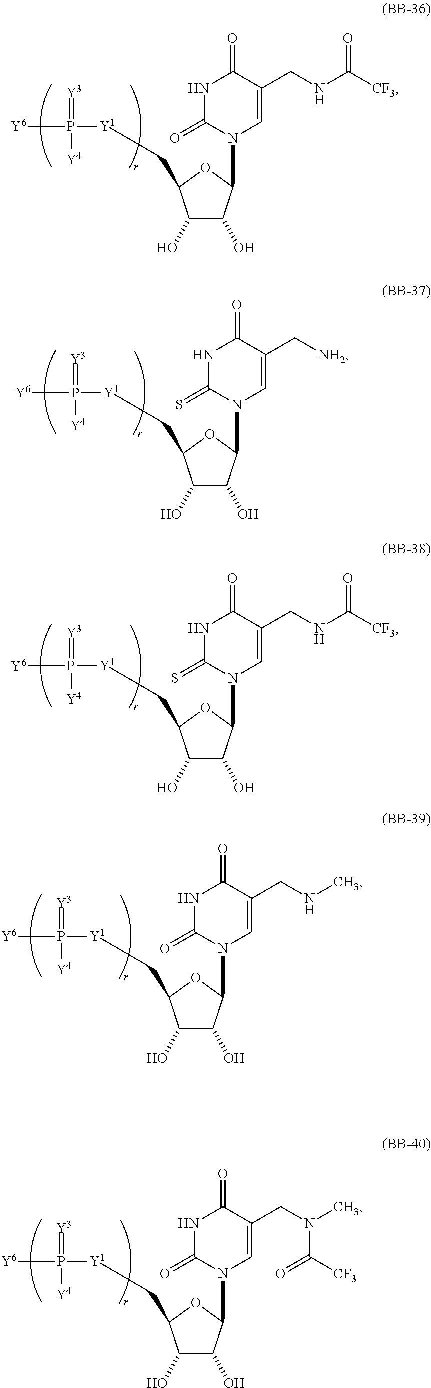

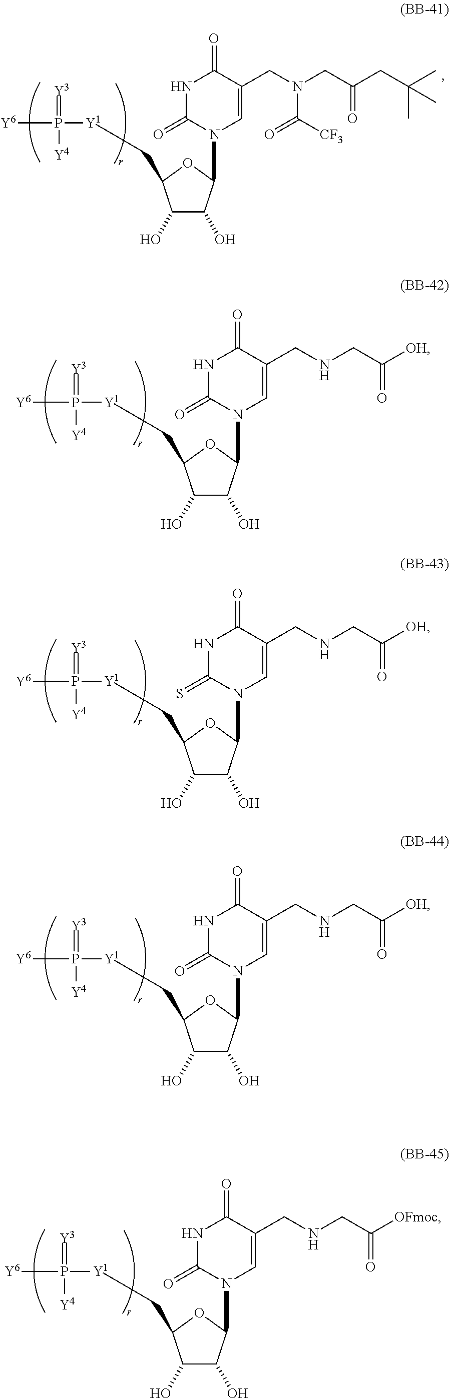

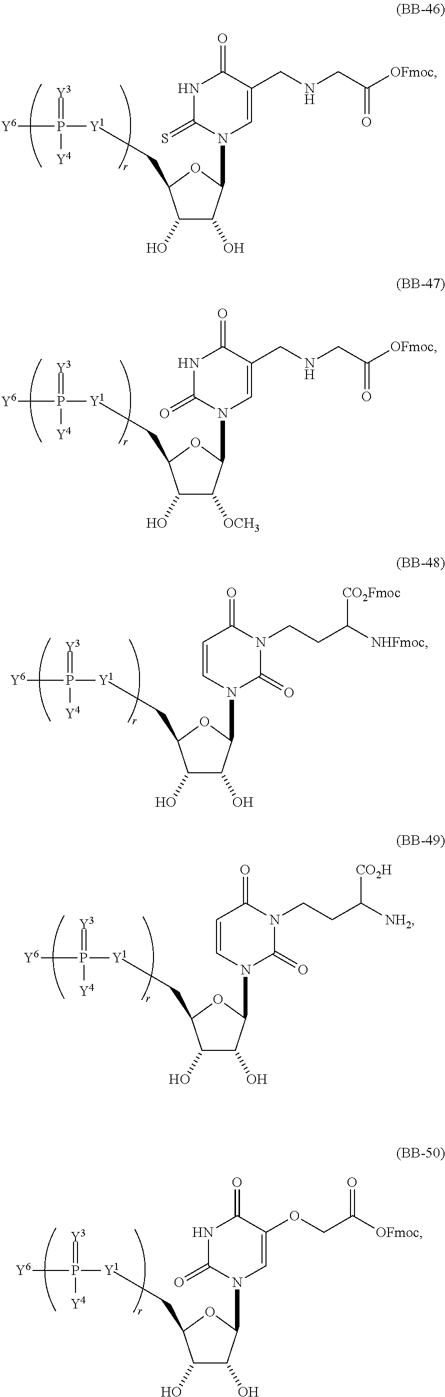

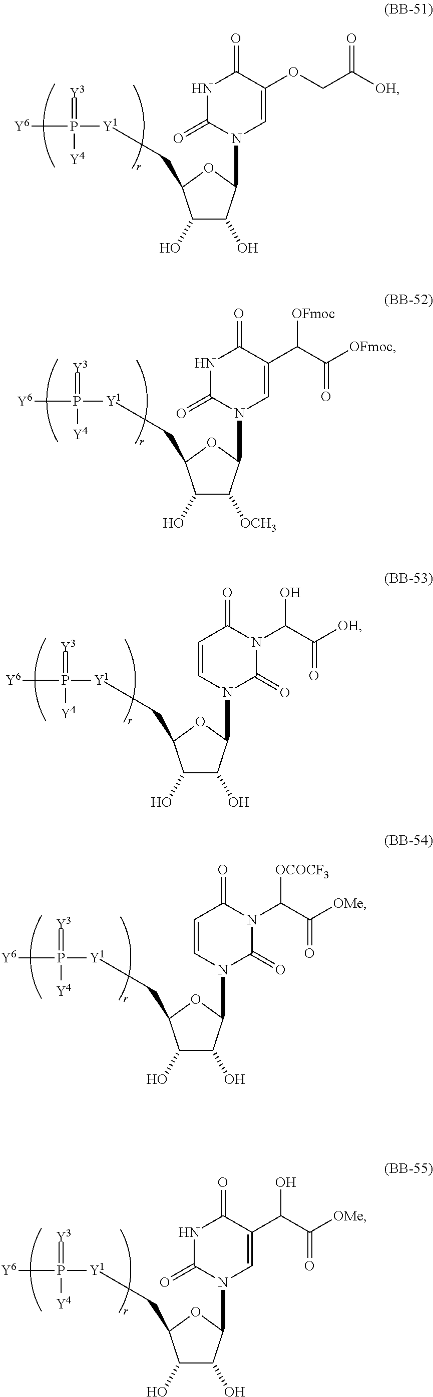

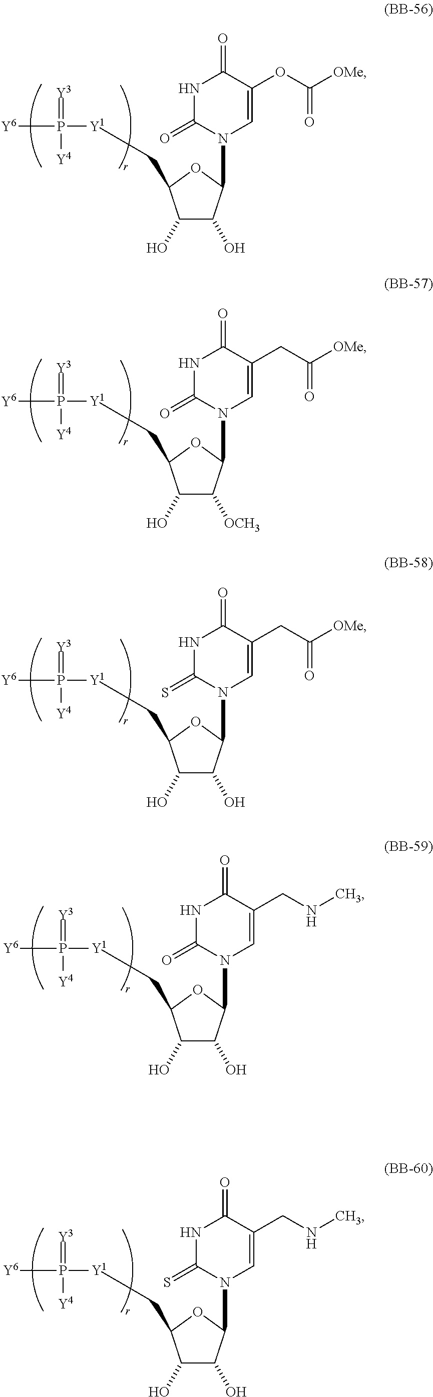

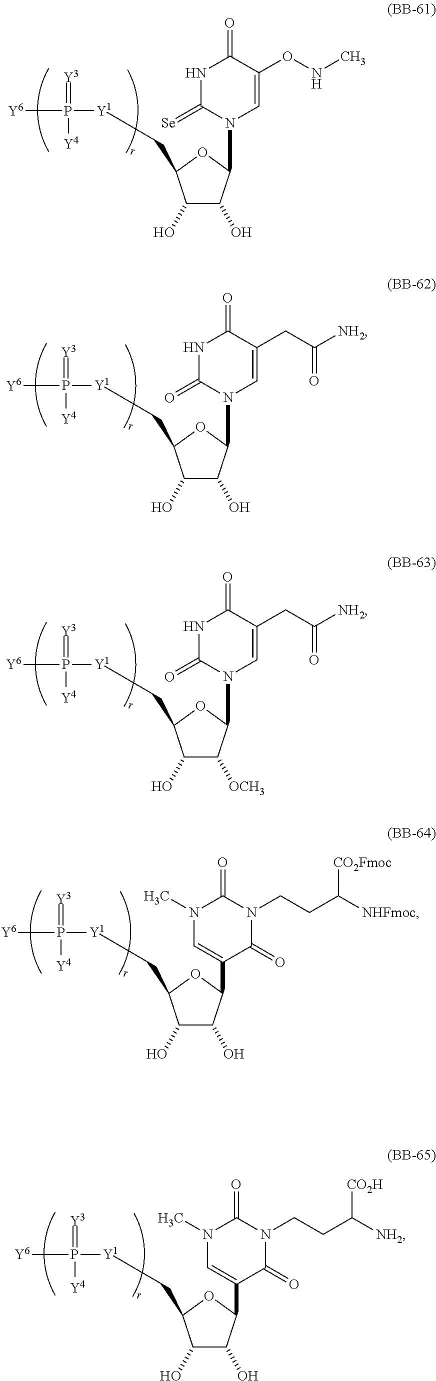

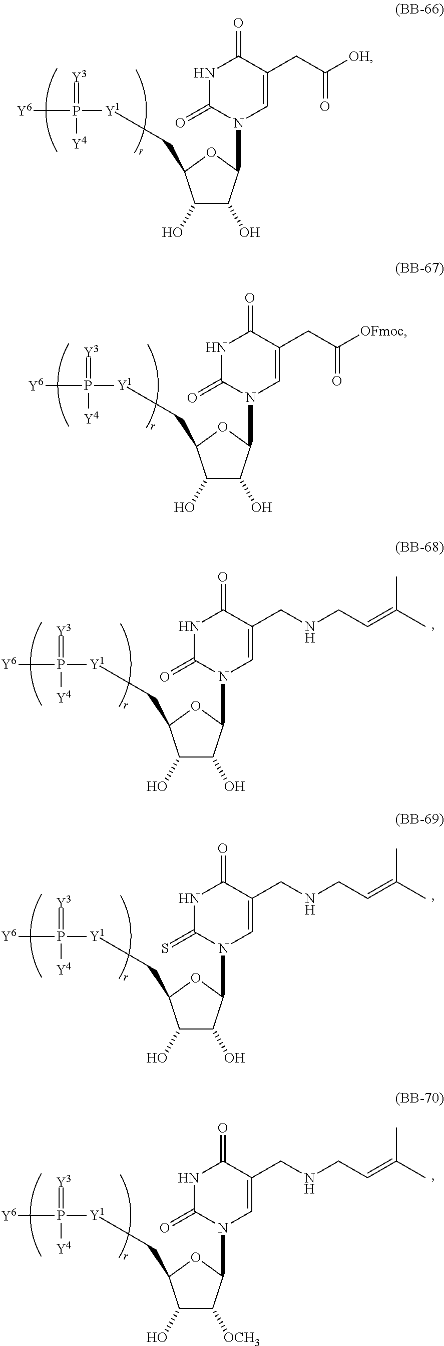

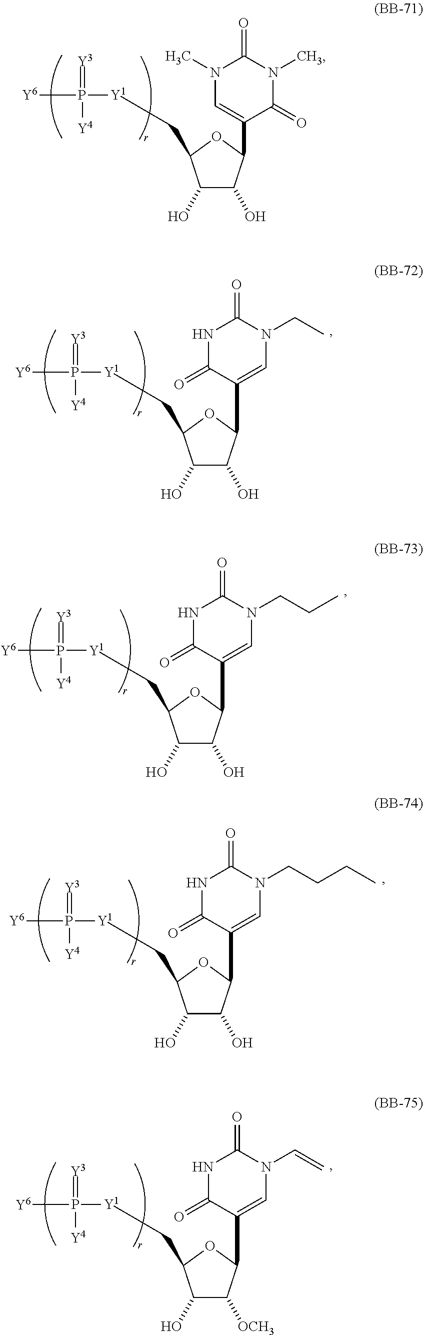

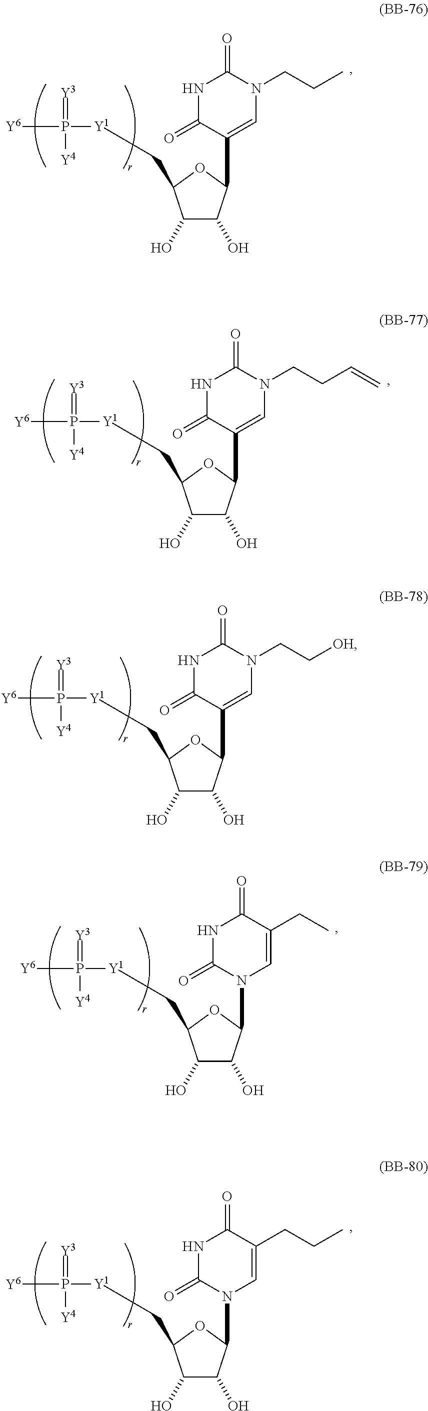

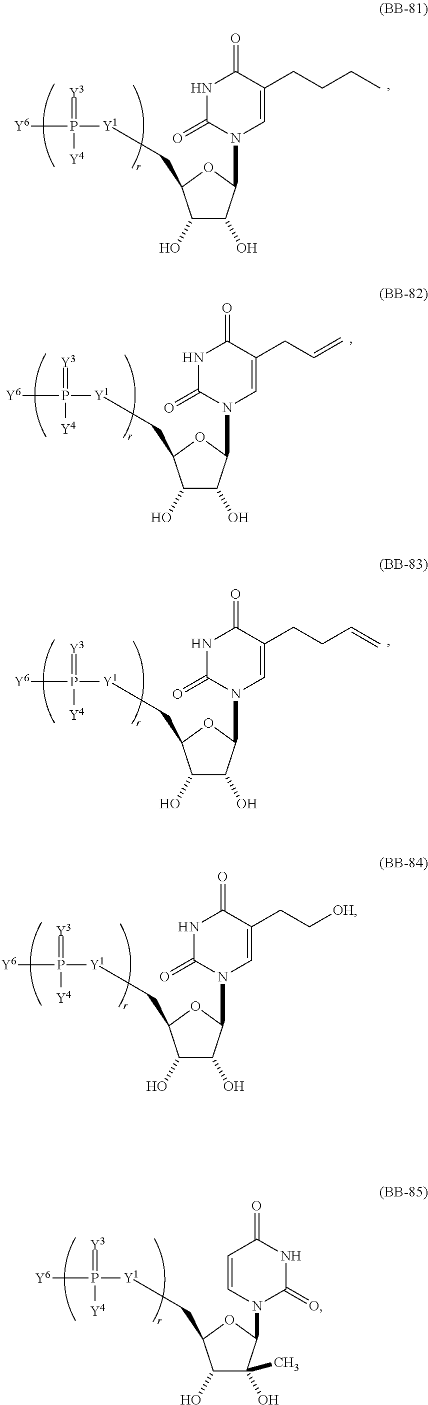

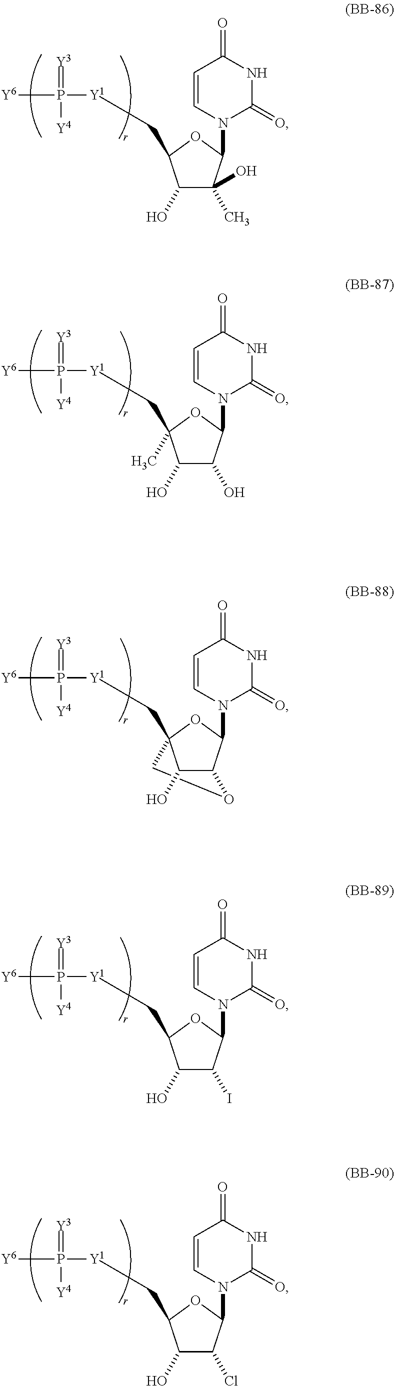

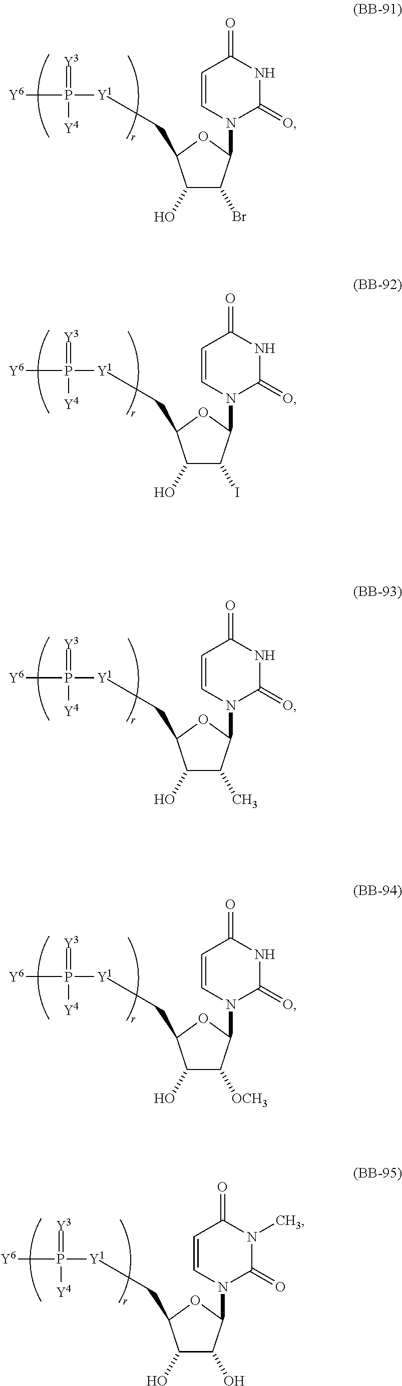

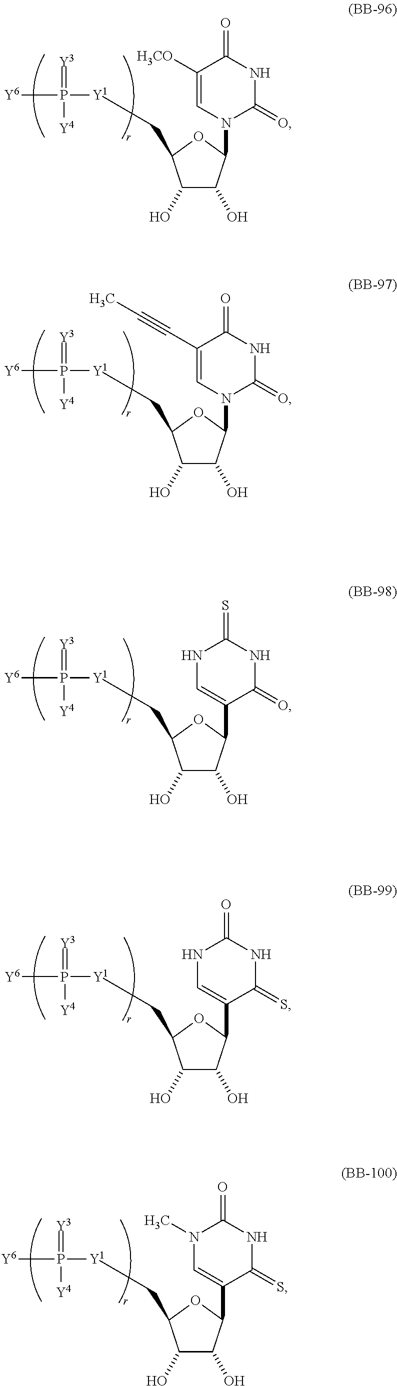

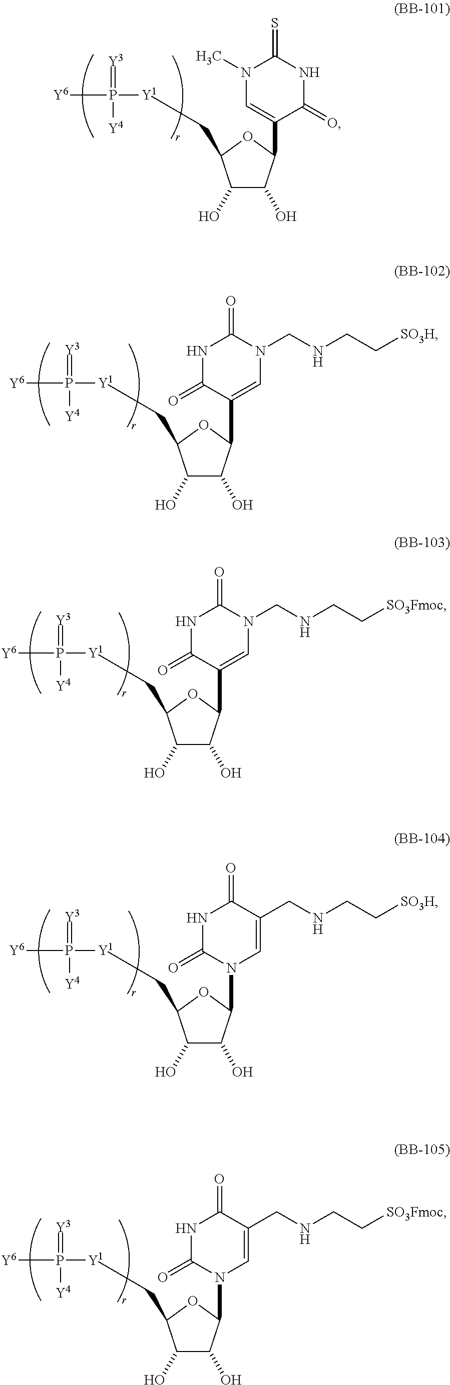

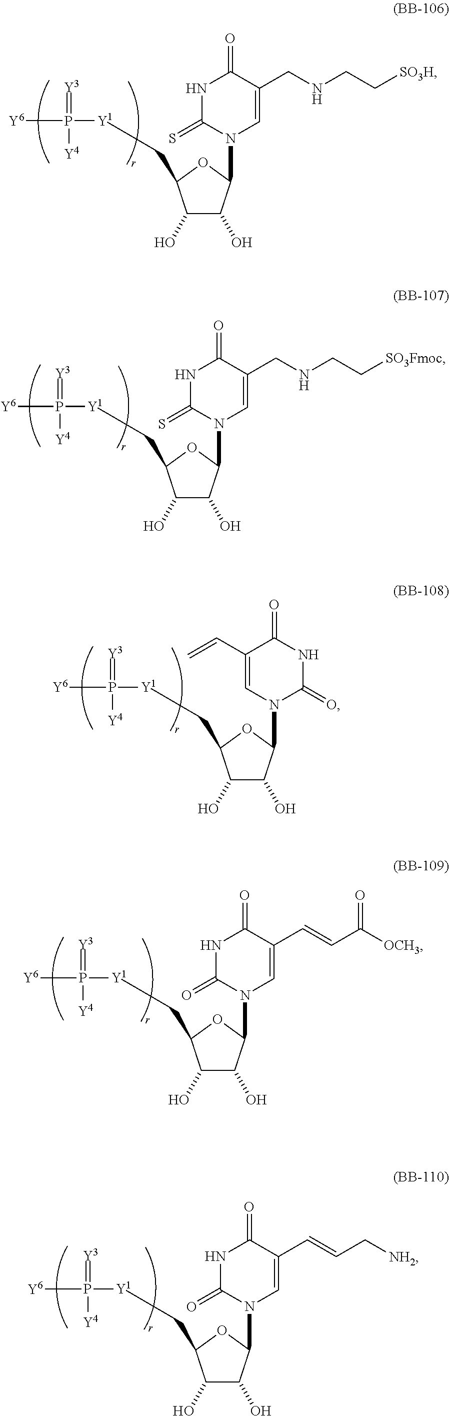

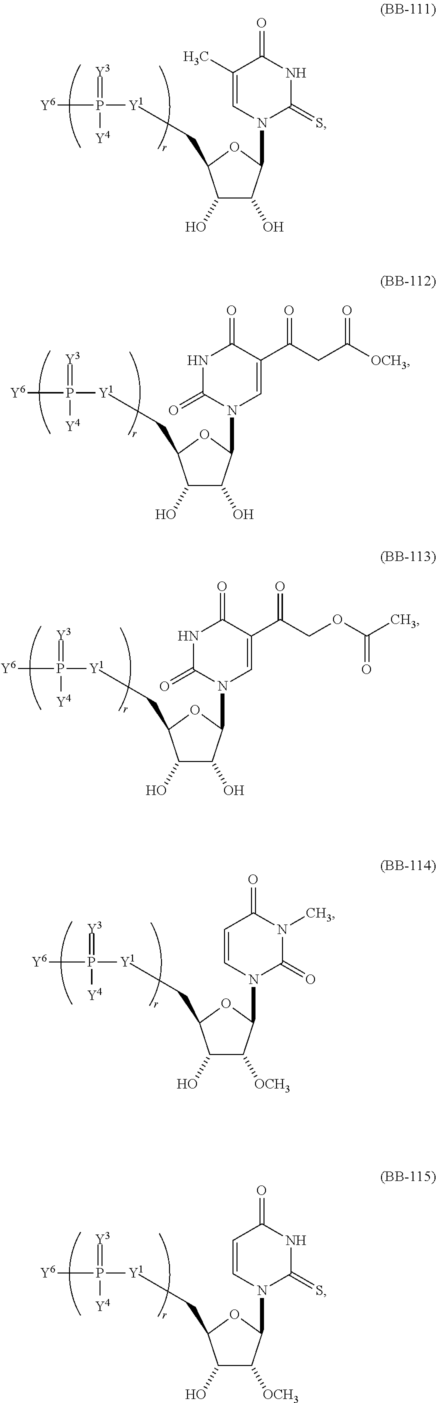

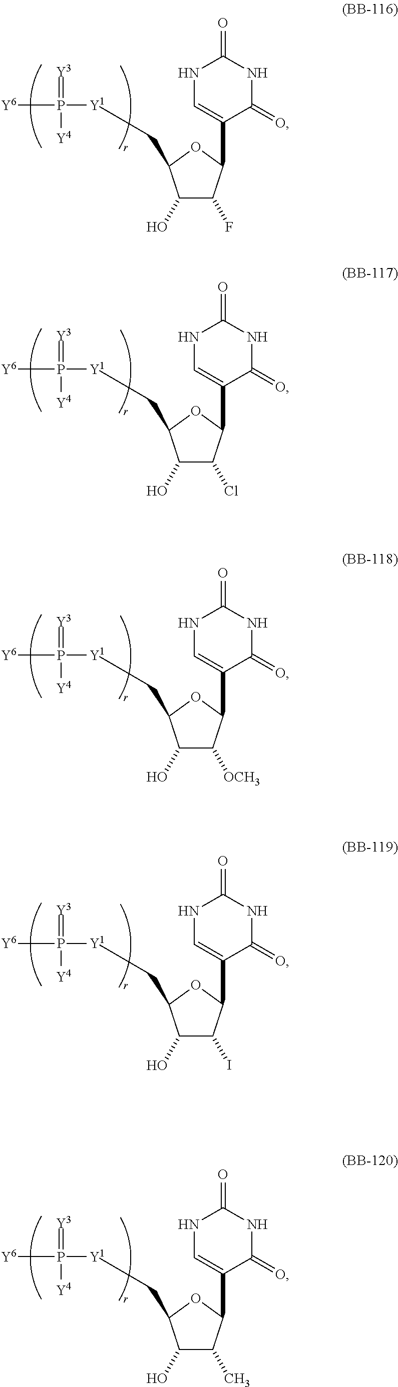

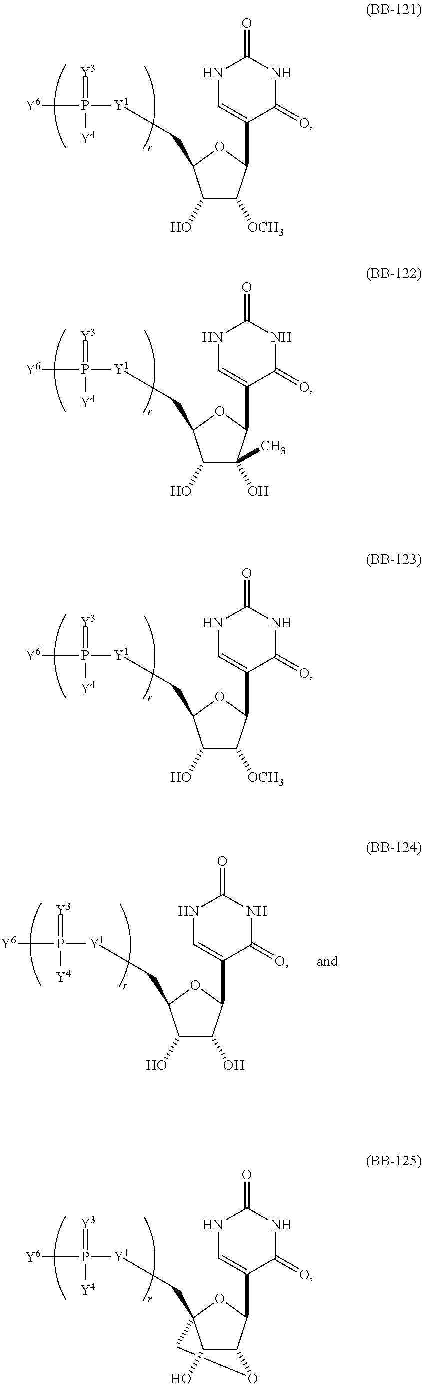

or a pharmaceutically acceptable salt or stereoisomer thereof, wherein Y¹, Y³, Y⁴, Y⁶, and r are as described herein (e.g., each r is, independently, an integer from 0 to 5, such as from 0 to 3, from 1 to 3, or from 1 to 5)).

In some embodiments, the building block molecule, which may be incorporated into a nucleic acids or modified RNA is a modified cytidine (e.g., selected from the group consisting of:

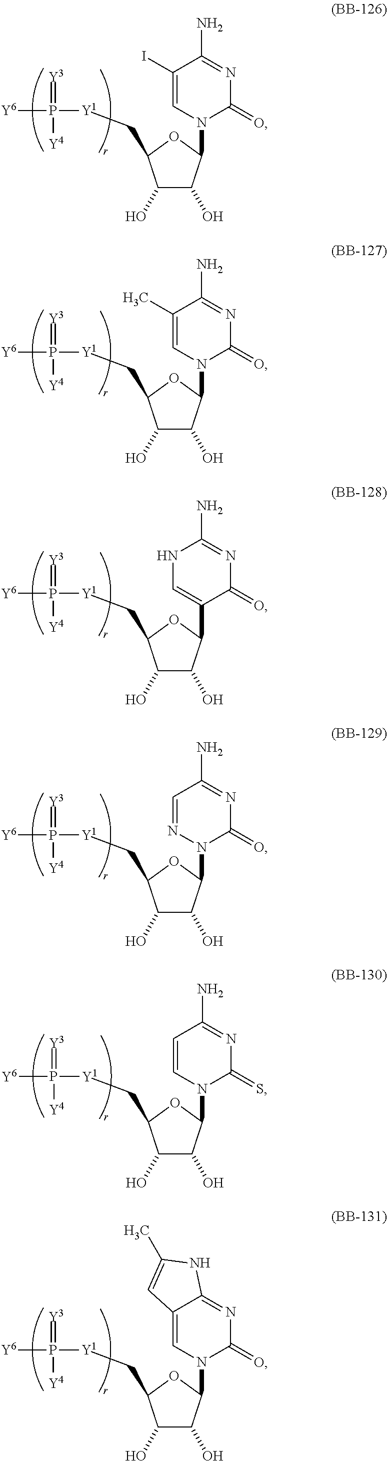

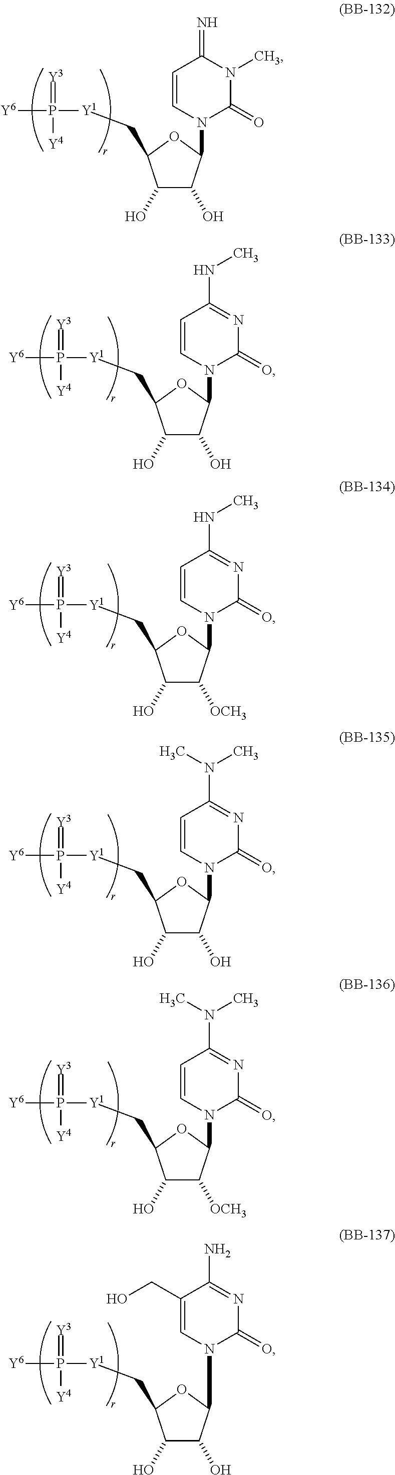

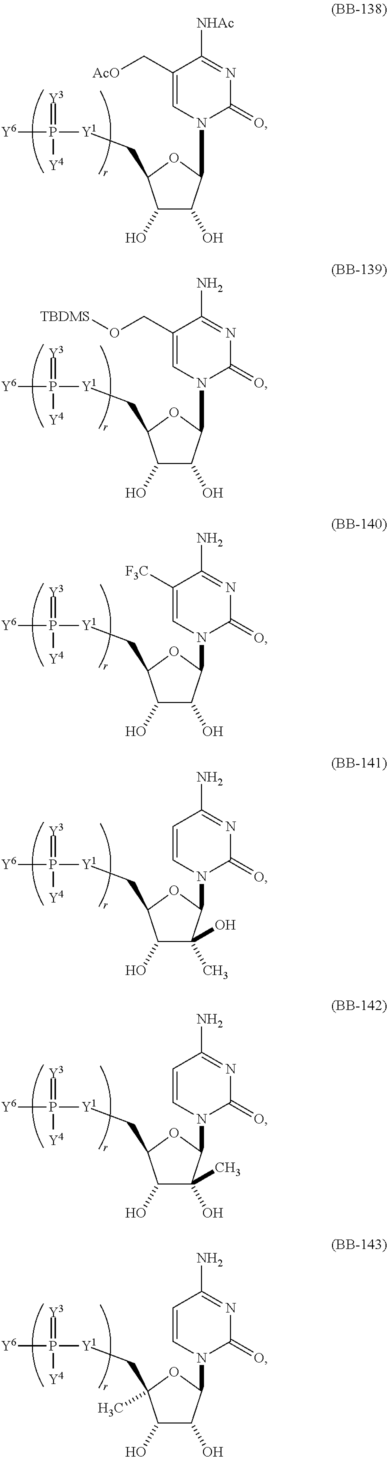

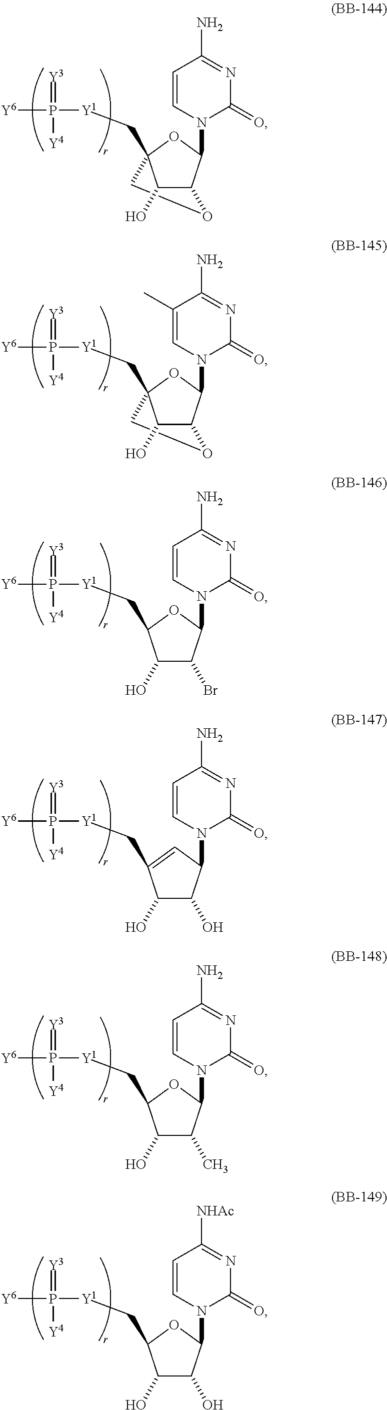

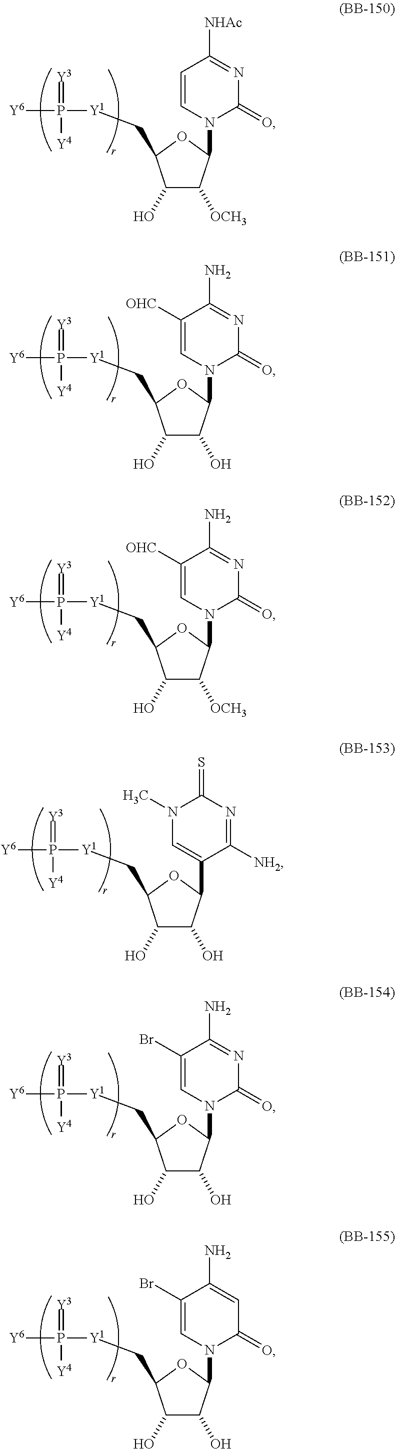

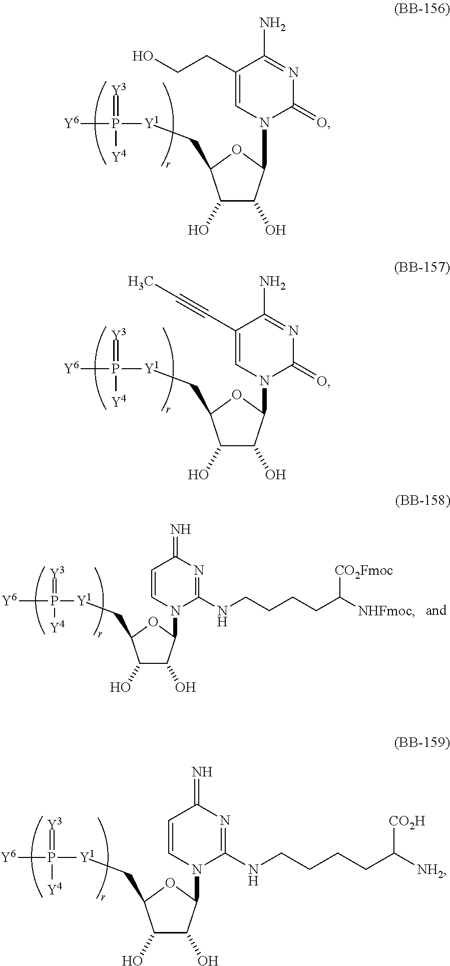

or a pharmaceutically acceptable salt or stereoisomer thereof, wherein Y¹, Y³, Y⁴, Y⁶, and r are as described herein (e.g., each r is, independently, an integer from 0 to 5, such as from 0 to 3, from 1 to 3, or from 1 to 5)). For example, the building block molecule, which may be incorporated into a nucleic acids or modified RNA can be:

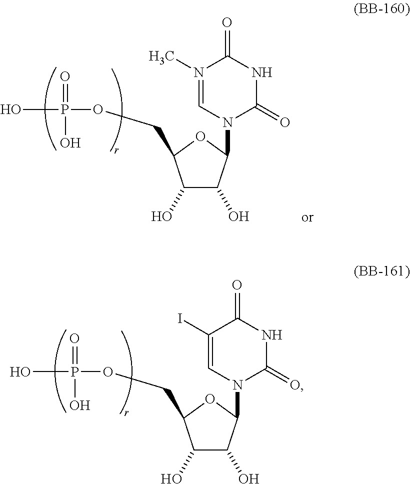

or a pharmaceutically acceptable salt or stereoisomer thereof, wherein each r is, independently, an integer from 0 to 5 (e.g., from 0 to 3, from 1 to 3, or from 1 to 5).

In some embodiments, the building block molecule, which may be incorporated into a nucleic acids or modified RNA is a modified adenosine (e.g., selected from the group consisting of:

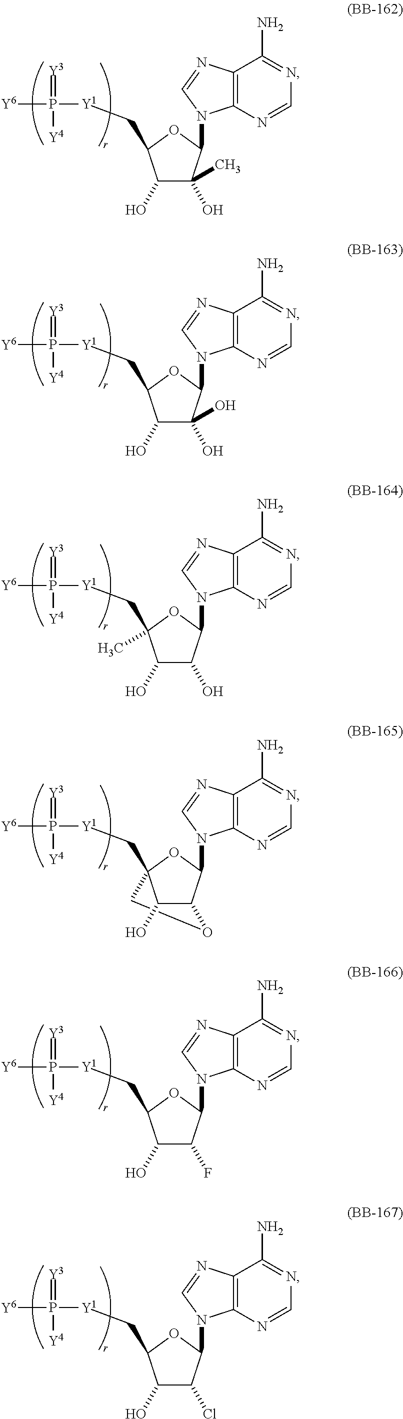

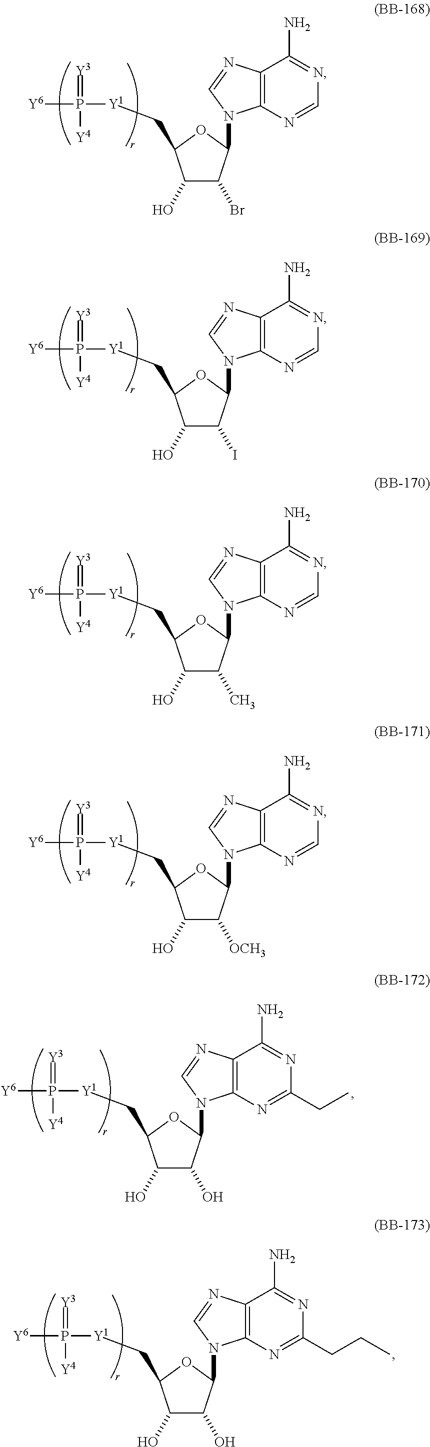

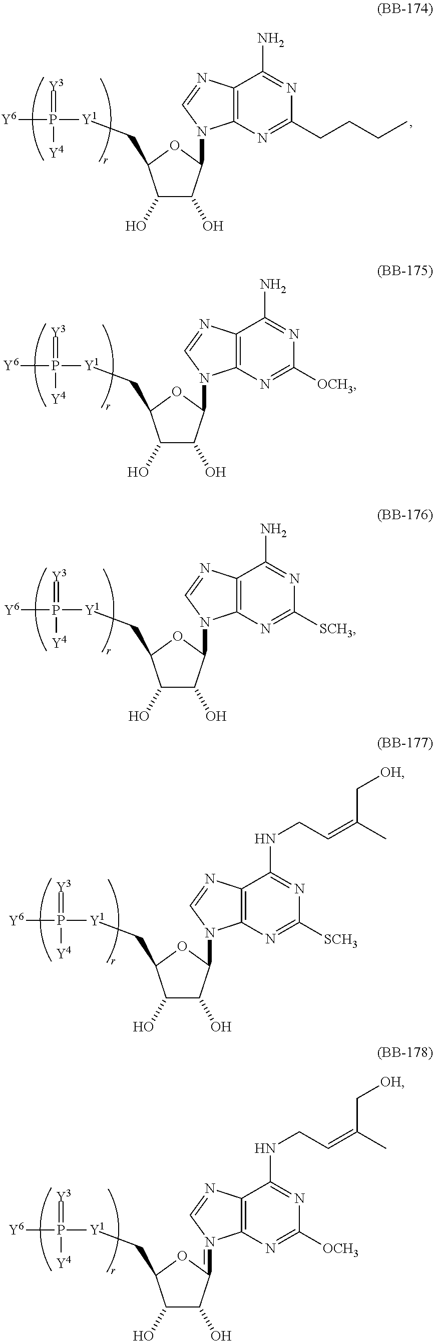

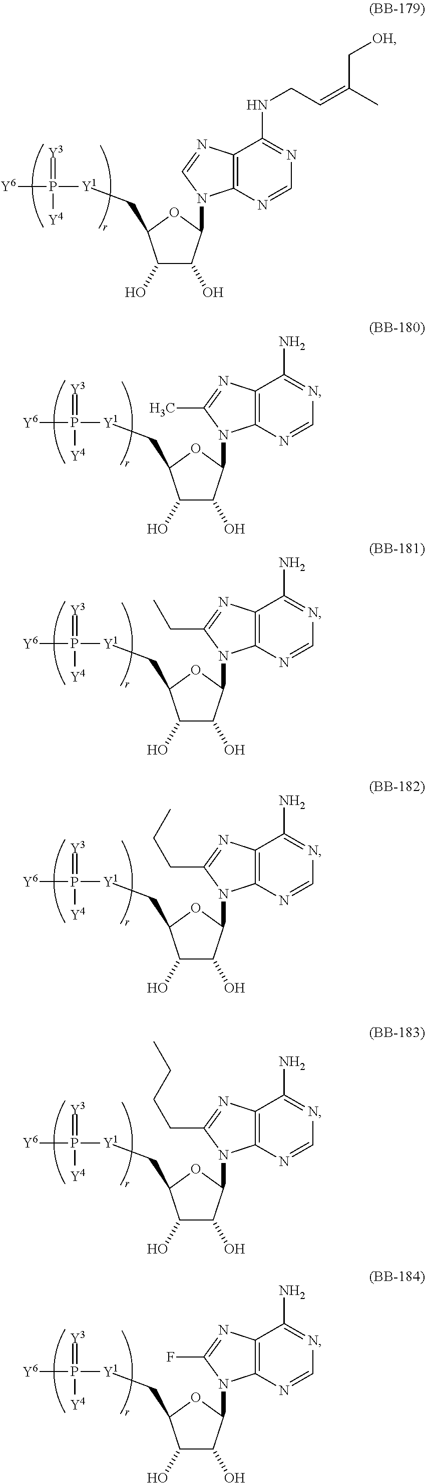

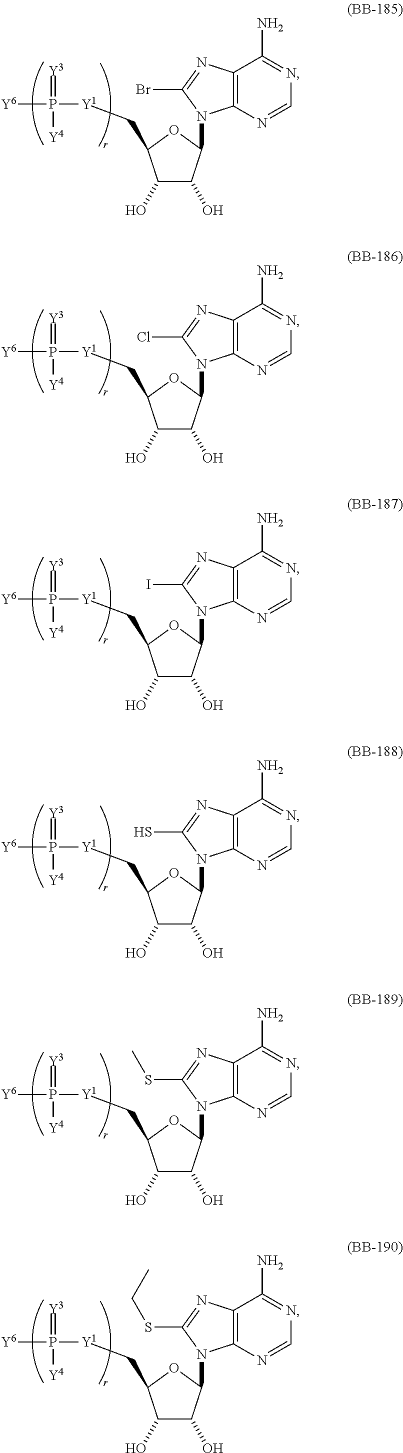

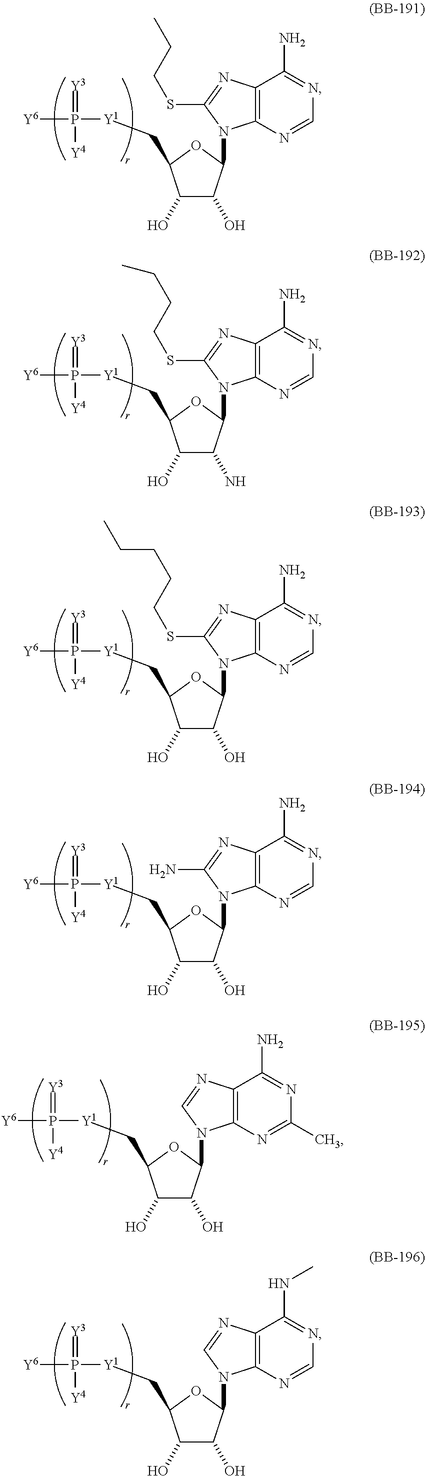

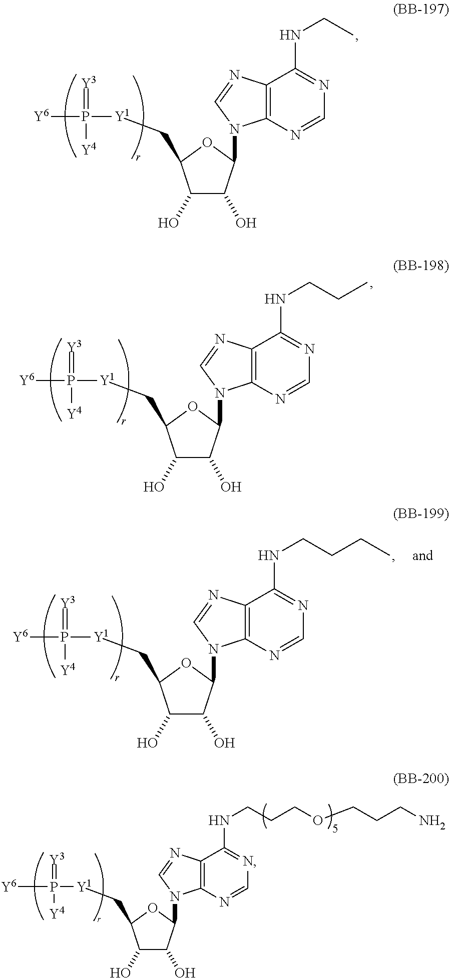

or a pharmaceutically acceptable salt or stereoisomer thereof, wherein Y¹, Y³, Y⁴, Y⁶, and r are as described herein (e.g., each r is, independently, an integer from 0 to 5, such as from 0 to 3, from 1 to 3, or from 1 to 5)).

In some embodiments, the building block molecule, which may be incorporated into a nucleic acids or modified RNA, is a modified guanosine (e.g., selected from the group consisting of:

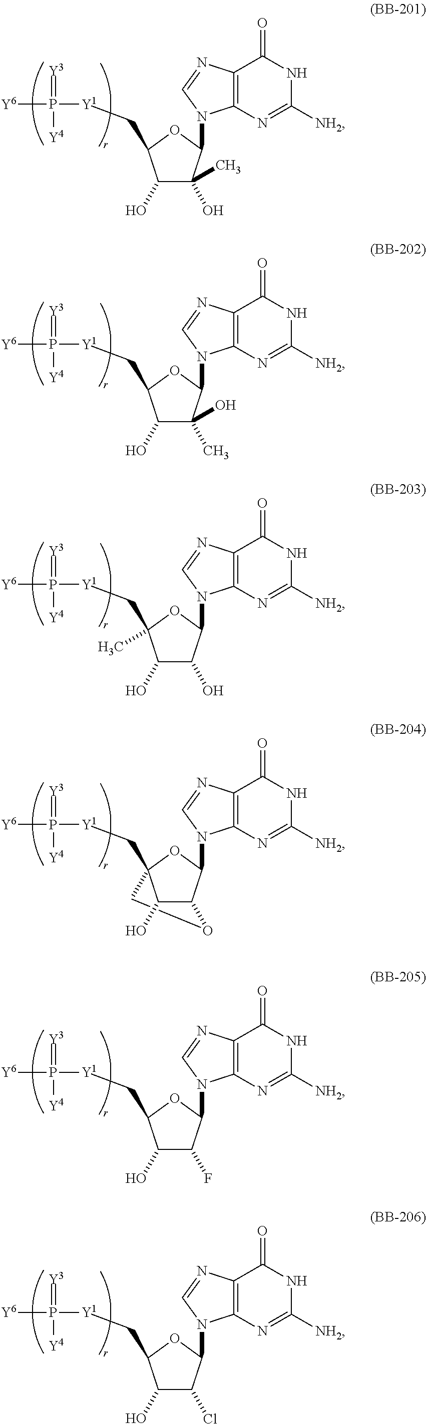

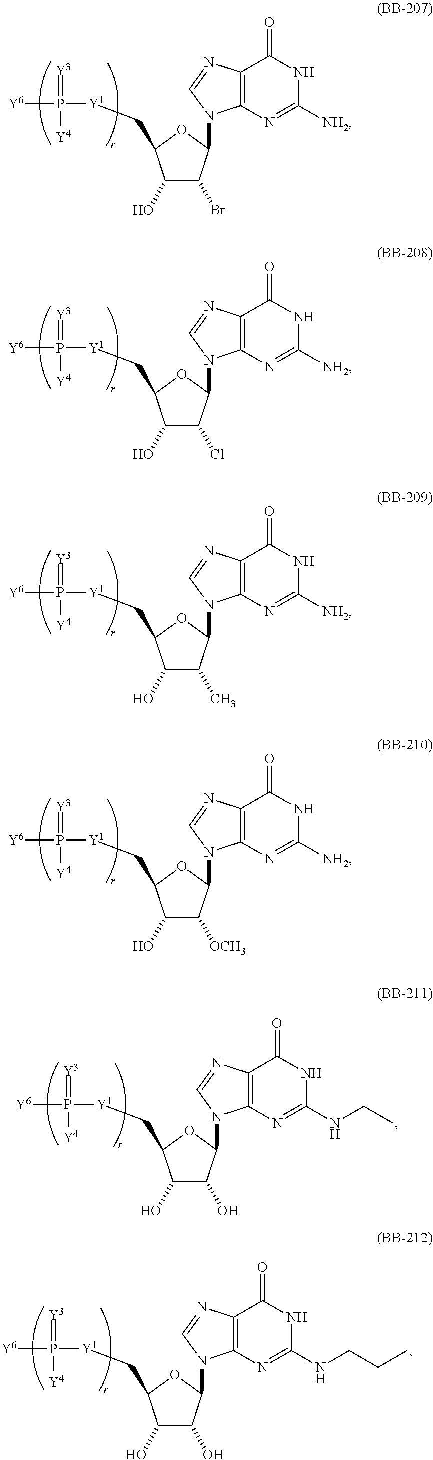

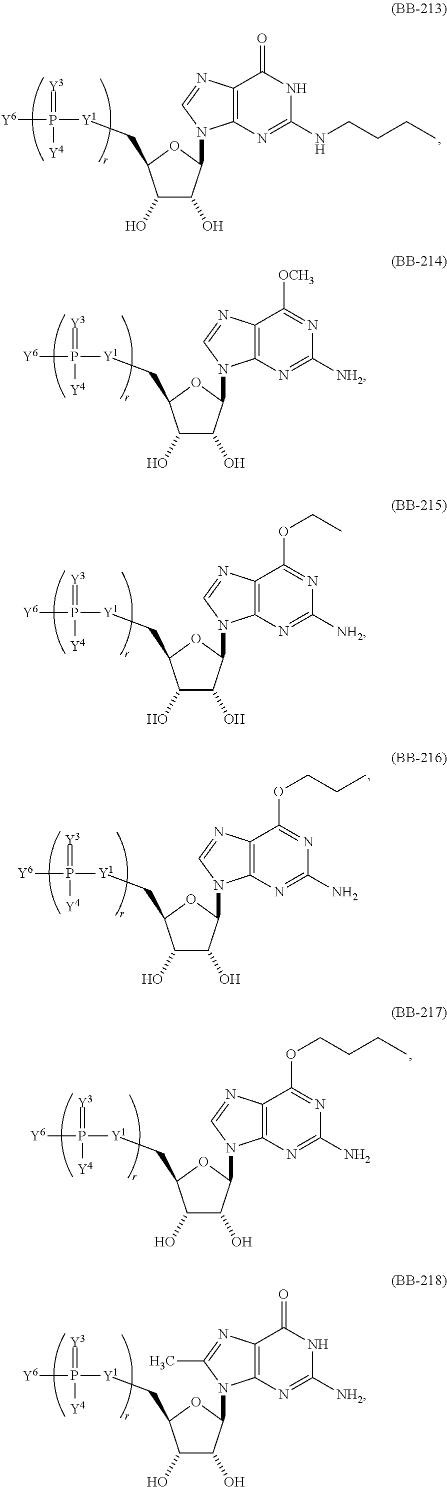

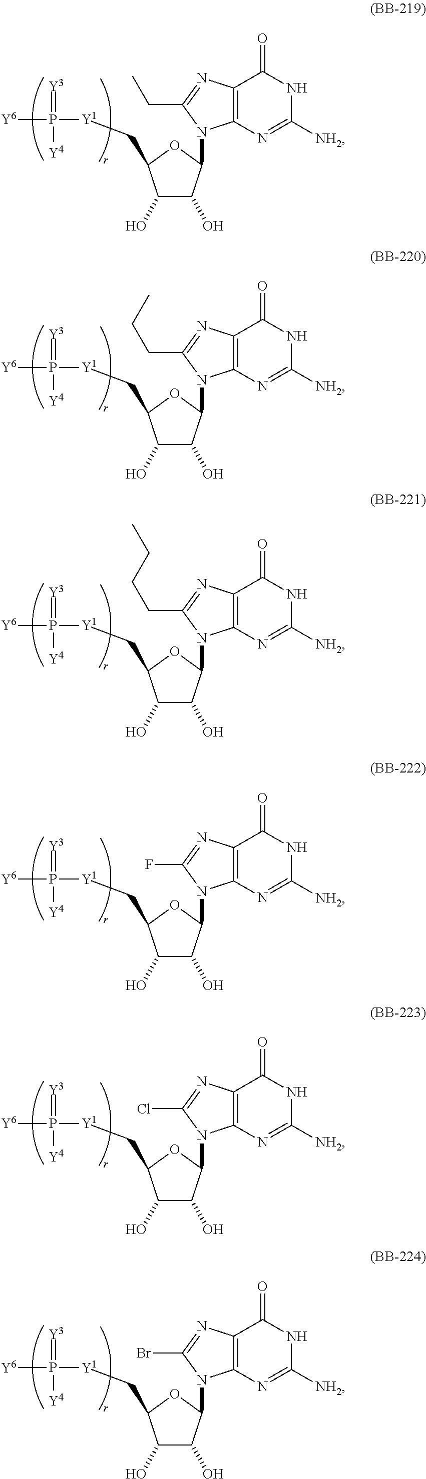

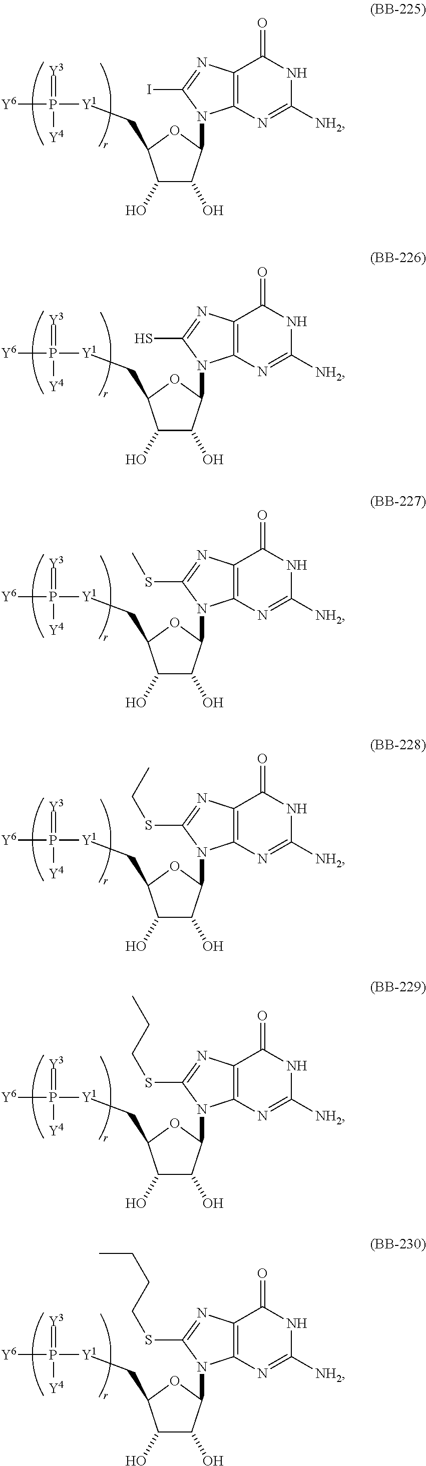

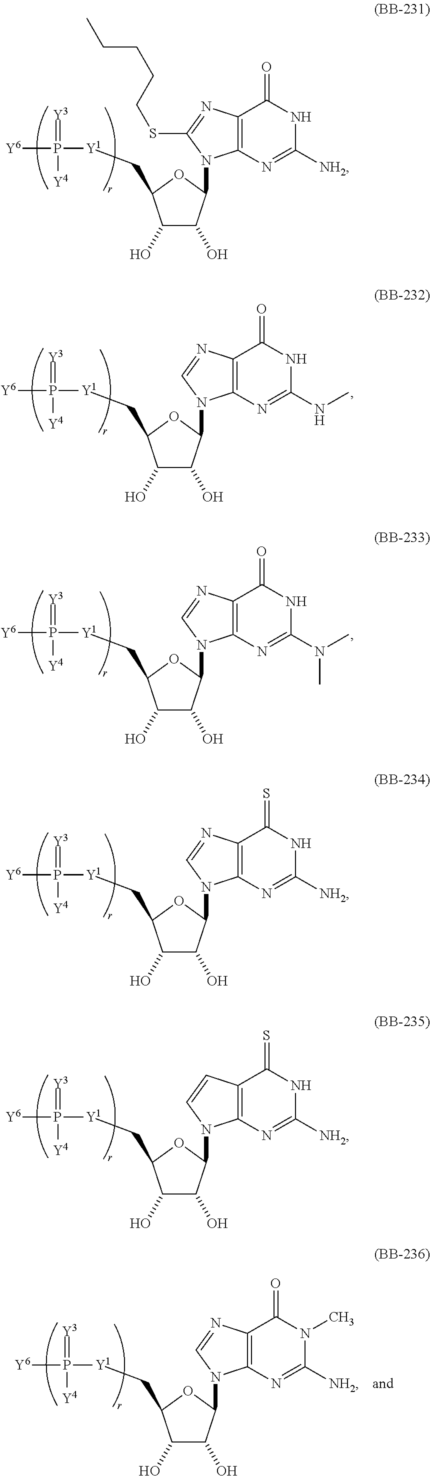

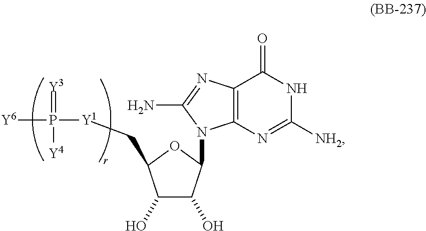

or a pharmaceutically acceptable salt or stereoisomer thereof, wherein Y¹, Y³, Y⁴, Y⁶, and r are as described herein (e.g., each r is, independently, an integer from 0 to 5, such as from 0 to 3, from 1 to 3, or from 1 to 5)).

In some embodiments, the chemical modification can include replacement of C group at C-5 of the ring (e.g., for a pyrimidine nucleoside, such as cytosine or uracil) with N (e.g., replacement of the >CH group at C-5 with >NR^{N1} group, wherein R^{N1} is H or optionally substituted alkyl). For example, the building block molecule, which may be incorporated into a nucleic acids or modified RNA can be:

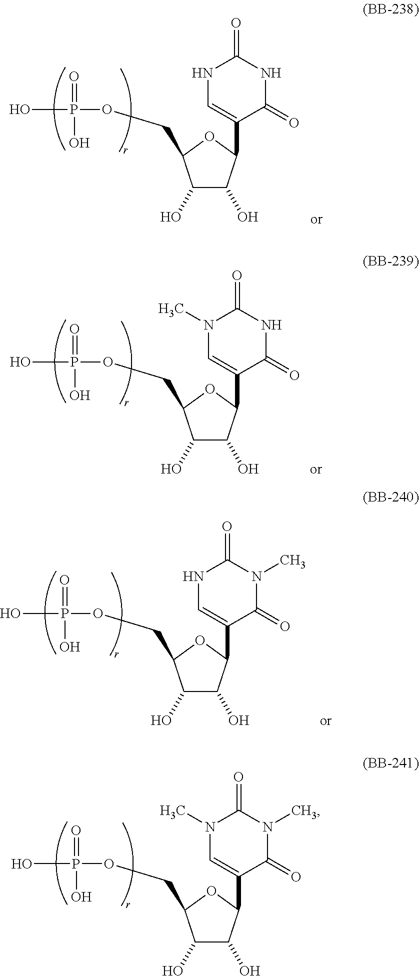

or a pharmaceutically acceptable salt or stereoisomer thereof, wherein each r is, independently, an integer from 0 to 5 (e.g., from 0 to 3, from 1 to 3, or from 1 to 5).

In another embodiment, the chemical modification can include replacement of the hydrogen at C-5 of cytosine with halo (e.g., Br, Cl, F, or I) or optionally substituted alkyl (e.g., methyl). For example, the building block molecule, which may be incorporated into a nucleic acids or modified RNA can be:

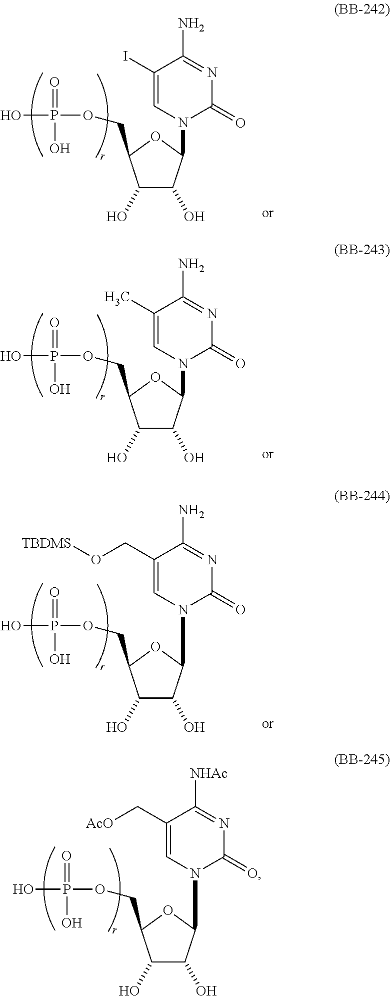

or a pharmaceutically acceptable salt or stereoisomer thereof, wherein each r is, independently, an integer from 0 to 5 (e.g., from 0 to 3, from 1 to 3, or from 1 to 5).

In yet a further embodiment, the chemical modification can include a fused ring that is formed by the NH₂ at the C-4 position and the carbon atom at the C-5 position. For example, the building block molecule, which may be incorporated into a nucleic acids or modified RNA can be:

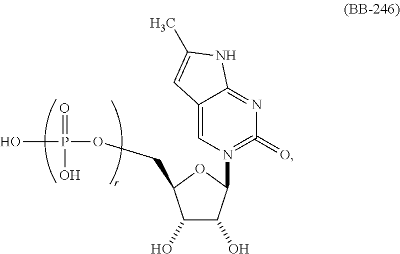

or a pharmaceutically acceptable salt or stereoisomer thereof, wherein each r is, independently, an integer from 0 to 5 (e.g., from 0 to 3, from 1 to 3, or from 1 to 5).

### Modifications on the Sugar

The modified nucleosides and nucleotides (e.g., building block molecules), which may be incorporated into a nucleic acids or modified RNA (e.g., RNA or mRNA, as described herein), can be modified on the sugar of the ribonucleic acid. For example, the 2′ hydroxyl group (OH) can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a nucleic acids or modified RNA molecule can include nucleotides containing, e.g., arabinose, as the sugar.

### Modifications on the Nucleobase

The present disclosure provides for modified nucleosides and nucleotides. As described herein “nucleoside” is defined as a compound containing a five-carbon sugar molecule (a pentose or ribose) or derivative thereof, and an organic base, purine or pyrimidine, or a derivative thereof. As described herein, “nucleotide” is defined as a nucleoside consisting of a phosphate group.

Exemplary non-limiting modifications include an amino group, a thiol group, an alkyl group, a halo group, or any described herein. The modified nucleotides may by synthesized by any useful method, as described herein (e.g., chemically, enzymatically, or recombinantly to include one or more modified or non-natural nucleosides).

The modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil.

The modified nucleosides and nucleotides can include a modified nucleobase. Examples of nucleobases found in RNA include, but are not limited to, adenine, guanine, cytosine, and uracil. Examples of nucleobase found in DNA include, but are not limited to, adenine, guanine, cytosine, and thymine. These nucleobases can be modified or wholly replaced to provide nucleic acids or modified RNA molecules having enhanced properties, e.g., resistance to nucleases, stability, and these properties may manifest through disruption of the binding of a major groove binding partner.

Table 2 below identifies the chemical faces of each canonical nucleotide. Circles identify the atoms comprising the respective chemical regions.

TABLE 2

| | | Major Groove Face | Minor Groove Face |
|---|---|---|---|
| Pyrimidines | Cytidine: | 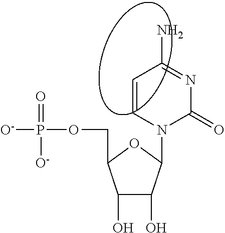 [SMILES: Nc1ccn(C2OC(COP(=O)([O-])[O-])C(O)C2O)c(=O)n1] | 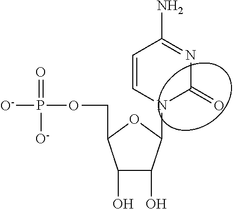 [SMILES: Nc1ccn(C2OC(COP(=O)([O-])[O-])C(O)C2O)c(=O)n1] |
| | Uridine: | 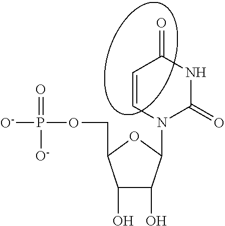 [SMILES: O=c1ccn(C2OC(COP(=O)([O-])[O-])C(O)C2O)c(=O)[nH]1] | 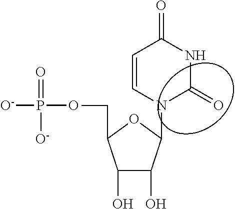 [SMILES: O=c1ccn(C2OC(COP(=O)([O-])[O-])C(O)C2O)c(=O)[nH]1] |
| Purines | Adenosine: | 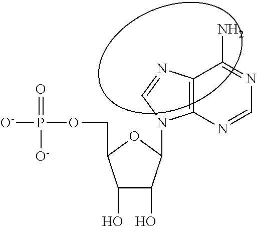 | 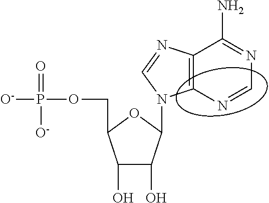 [SMILES: Nc1ncnc2c1ncn2C1OC(COP(=O)([O-])[O-])C(O)C1O] |
| | Guanosine: | 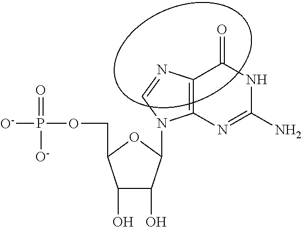 [SMILES: Nc1nc2c(ncn2C2OC(COP(=O)([O-])[O-])C(O)C2O)c(=O)[nH]1] | 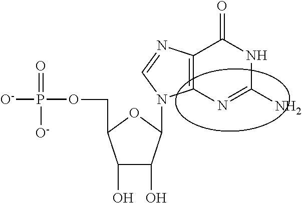 [SMILES: Nc1nc2c(ncn2C2OC(COP(=O)([O-])[O-])C(O)C2O)c(=O)[nH]1] |

| | | Watson-Crick Base-pairing Face |
|---|---|---|
| Pyrimidines | Cytidine: | 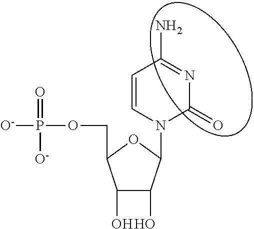 [SMILES: Nc1ccn(C2OC(COP(=O)([O-])[O-])C(O)C2O)c(=O)n1] |
| | Uridine: | 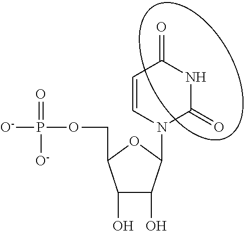 [SMILES: O=c1ccn(C2OC(COP(=O)([O-])[O-])C(O)C2O)c(=O)[nH]1] |
| Purines | Adenosine: | 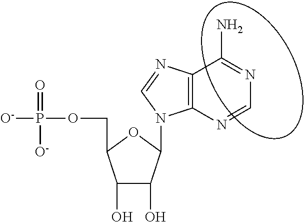 [SMILES: Nc1ncnc2c1ncn2C1OC(COP(=O)([O-])[O-])C(O)C1O] |
| | Guanosine: | 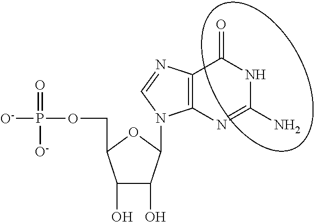 [SMILES: Nc1nc2c(ncn2C2OC(COP(=O)([O-])[O-])C(O)C2O)c(=O)[nH]1] |

In some embodiments, B is a modified uracil. Exemplary modified uracils include those having Formula (b1)-(b5):

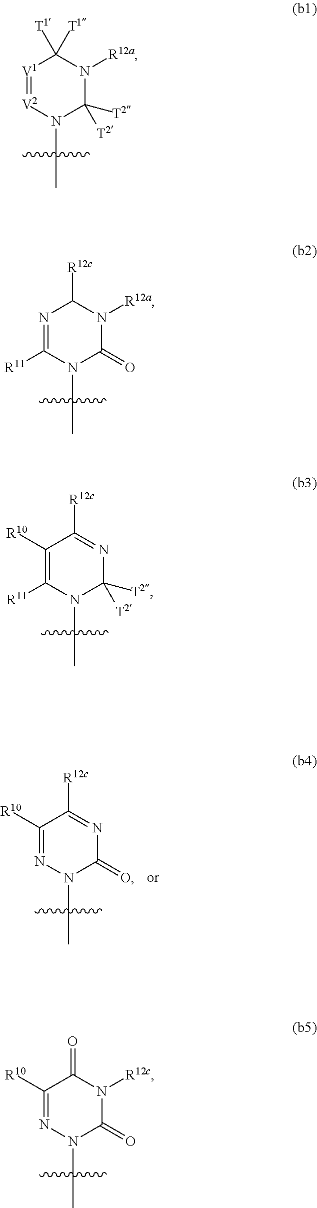

or a pharmaceutically acceptable salt or stereoisomer thereof, wherein

[symbol] is a single or double bond;

each of T^{1′}, T^{1″}, T^{2′}, and T^{2″} is, independently, H, optionally substituted alkyl, optionally substituted alkoxy, or optionally substituted thioalkoxy, or the combination of T^{1′} and T^{1″} or the combination of T^{2′} and T^{2″} join together (e.g., as in T²) to form O (oxo), S (thio), or Se (seleno);

each of V¹ and V² is, independently, O, S, N(R^{Vb})ₙᵥ, or C(R^{Vb})ₙᵥ, wherein nv is an integer from 0 to 2 and each R^{Vb} is, independently, H, halo, optionally substituted amino acid, optionally substituted alkyl, optionally substituted haloalkyl, optionally substituted alkenyl, optionally substituted alkynyl, optionally substituted alkoxy, optionally substituted alkenyloxy, optionally substituted alkynyloxy, optionally substituted hydroxyalkyl, optionally substituted hydroxyalkenyl, optionally substituted hydroxyalkynyl, optionally substituted aminoalkyl (e.g., substituted with an N-protecting group, such as any described herein, e.g., trifluoroacetyl), optionally substituted aminoalkenyl, optionally substituted aminoalkynyl, optionally substituted acylaminoalkyl (e.g., substituted with an N-protecting group, such as any described herein, e.g., trifluoroacetyl), optionally substituted alkoxycarbonylalkyl, optionally substituted alkoxycarbonylalkenyl, optionally substituted alkoxycarbonylalkynyl, or optionally substituted alkoxycarbonylalkoxy (e.g., optionally substituted with any substituent described herein, such as those selected from (1)-(21) for alkyl);

R¹⁰ is H, halo, optionally substituted amino acid, hydroxy, optionally substituted alkyl, optionally substituted alkenyl, optionally substituted alkynyl, optionally substituted aminoalkyl, optionally substituted hydroxyalkyl, optionally substituted hydroxyalkenyl, optionally substituted hydroxyalkynyl, optionally substituted aminoalkenyl, optionally substituted aminoalkynyl, optionally substituted alkoxy, optionally substituted alkoxycarbonylalkyl, optionally substituted alkoxycarbonylalkenyl, optionally substituted alkoxycarbonylalkynyl, optionally substituted alkoxycarbonylalkoxy, optionally substituted carboxyalkoxy, optionally substituted carboxyalkyl, or optionally substituted carbamoylalkyl;

R¹¹ is H or optionally substituted alkyl;

R^{12a} is H, optionally substituted alkyl, optionally substituted hydroxyalkyl, optionally substituted hydroxyalkenyl, optionally substituted hydroxyalkynyl, optionally substituted aminoalkyl, optionally substituted aminoalkenyl, or optionally substituted aminoalkynyl, optionally substituted carboxyalkyl (e.g., optionally substituted with hydroxy), optionally substituted carboxyalkoxy, optionally substituted carboxyaminoalkyl, or optionally substituted carbamoylalkyl; and

R^{12c} is H, halo, optionally substituted alkyl, optionally substituted alkoxy, optionally substituted thioalkoxy, optionally substituted amino, optionally substituted hydroxyalkyl, optionally substituted hydroxyalkenyl, optionally substituted hydroxyalkynyl, optionally substituted aminoalkyl, optionally substituted aminoalkenyl, or optionally substituted aminoalkynyl.

Other exemplary modified uracils include those having Formula (b6)-(b9):

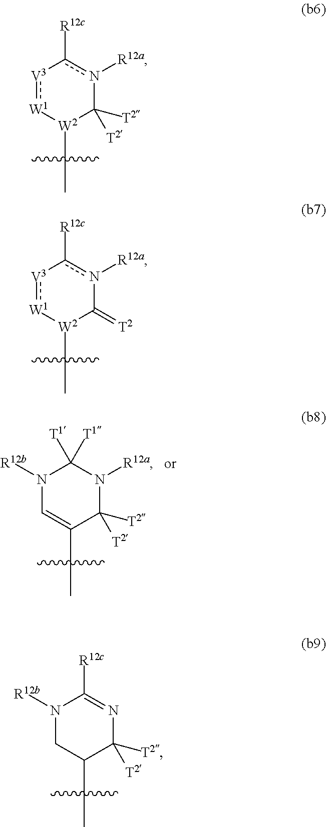

or a pharmaceutically acceptable salt or stereoisomer thereof, wherein

[symbol] is a single or double bond;

each of T^{1′}, T^{1″}, T^{2′}, and T^{2″} is, independently, H, optionally substituted alkyl, optionally substituted alkoxy, or optionally substituted thioalkoxy, or the combination of T^{1′} and T^{1″} join together (e.g., as in T¹) or the combination of T^{2′} and T^{2″} join together (e.g., as in T²) to form O (oxo), S (thio), or Se (seleno), or each T¹ and T² is, independently, O (oxo), S (thio), or Se (seleno);

each of W¹ and W² is, independently, N(R^{Wa})_{nw} or C(R^{Wa})_{nw}, wherein nw is an integer from 0 to 2 and each R^{Wa} is, independently, H, optionally substituted alkyl, or optionally substituted alkoxy;

each V³ is, independently, O, S, N(R^{Va})ₙᵥ, or C(R^{Va})ₙᵥ, wherein nv is an integer from 0 to 2 and each R^{Va} is, independently, H, halo, optionally substituted amino acid, optionally substituted alkyl, optionally substituted hydroxyalkyl, optionally substituted hydroxyalkenyl, optionally substituted hydroxyalkynyl, optionally substituted alkenyl, optionally substituted alkynyl, optionally substituted heterocyclyl, optionally substituted alkheterocyclyl, optionally substituted alkoxy, optionally substituted alkenyloxy, or optionally substituted alkynyloxy, optionally substituted aminoalkyl (e.g., substituted with an N-protecting group, such as any described herein, e.g., trifluoroacetyl, or sulfoalkyl), optionally substituted aminoalkenyl, optionally substituted aminoalkynyl, optionally substituted acylaminoalkyl (e.g., substituted with an N-protecting group, such as any described herein, e.g., trifluoroacetyl), optionally substituted alkoxycarbonylalkyl, optionally substituted alkoxycarbonylalkenyl, optionally substituted alkoxycarbonylalkynyl, optionally substituted alkoxycarbonylacyl, optionally substituted alkoxycarbonylalkoxy, optionally substituted carboxyalkyl (e.g., optionally substituted with hydroxy and/or an O-protecting group), optionally substituted carboxyalkoxy, optionally substituted carboxyaminoalkyl, or optionally substituted carbamoylalkyl (e.g., optionally substituted with any substituent described herein, such as those selected from (1)-(21) for alkyl), and wherein R^{Va} and R^{12c} taken together with the carbon atoms to which they are attached can form optionally substituted cycloalkyl, optionally substituted aryl, or optionally substituted heterocyclyl (e.g., a 5- or 6-membered ring);

R^{12a} is H, optionally substituted alkyl, optionally substituted hydroxyalkyl, optionally substituted hydroxyalkenyl, optionally substituted hydroxyalkynyl, optionally substituted aminoalkyl, optionally substituted aminoalkenyl, optionally substituted aminoalkynyl, optionally substituted carboxyalkyl (e.g., optionally substituted with hydroxy and/or an O-protecting group), optionally substituted carboxyalkoxy, optionally substituted carboxyaminoalkyl, optionally substituted carbamoylalkyl, or absent;

R^{12b} is H, optionally substituted alkyl, optionally substituted alkenyl, optionally substituted alkynyl, optionally substituted hydroxyalkyl, optionally substituted hydroxyalkenyl, optionally substituted hydroxyalkynyl, optionally substituted aminoalkyl, optionally substituted aminoalkenyl, optionally substituted aminoalkynyl, optionally substituted alkaryl, optionally substituted heterocyclyl, optionally substituted alkheterocyclyl, optionally substituted amino acid, optionally substituted alkoxycarbonylacyl, optionally substituted alkoxycarbonylalkoxy, optionally substituted alkoxycarbonylalkyl, optionally substituted alkoxycarbonylalkenyl, optionally substituted alkoxycarbonylalkynyl, optionally substituted alkoxycarbonylalkoxy, optionally substituted carboxyalkyl (e.g., optionally substituted with hydroxy and/or an O-protecting group), optionally substituted carboxyalkoxy, optionally substituted carboxyaminoalkyl, or optionally substituted carbamoylalkyl,

wherein the combination of R^{12b} and T^{1′} or the combination of R^{12b} and R^{12c} can join together to form optionally substituted heterocyclyl; and

R^{12c} is H, halo, optionally substituted alkyl, optionally substituted alkoxy, optionally substituted thioalkoxy, optionally substituted amino, optionally substituted aminoalkyl, optionally substituted aminoalkenyl, or optionally substituted aminoalkynyl.

Further exemplary modified uracils include those having Formula (b28)-(b31):

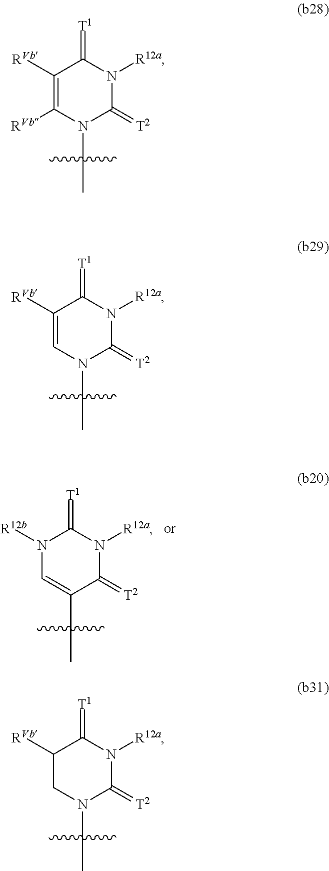

or a pharmaceutically acceptable salt or stereoisomer thereof, wherein

each of T¹ and T² is, independently, O (oxo), S (thio), or Se (seleno);

each R^{Vb′} and R^{Vb″} is, independently, H, halo, optionally substituted amino acid, optionally substituted alkyl, optionally substituted haloalkyl, optionally substituted hydroxyalkyl, optionally substituted hydroxyalkenyl, optionally substituted hydroxyalkynyl, optionally substituted alkenyl, optionally substituted alkynyl, optionally substituted alkoxy, optionally substituted alkenyloxy, optionally substituted alkynyloxy, optionally substituted aminoalkyl (e.g., substituted with an N-protecting group, such as any described herein, e.g., trifluoroacetyl, or sulfoalkyl), optionally substituted aminoalkenyl, optionally substituted aminoalkynyl, optionally substituted acylaminoalkyl (e.g., substituted with an N-protecting group, such as any described herein, e.g., trifluoroacetyl), optionally substituted alkoxycarbonylalkyl, optionally substituted alkoxycarbonylalkenyl, optionally substituted alkoxycarbonylalkynyl, optionally substituted alkoxycarbonylacyl, optionally substituted alkoxycarbonylalkoxy, optionally substituted carboxyalkyl (e.g., optionally substituted with hydroxy and/or an O-protecting group), optionally substituted carboxyalkoxy, optionally substituted carboxyaminoalkyl, or optionally substituted carbamoylalkyl (e.g., optionally substituted with any substituent described herein, such as those selected from (1)-(21) for alkyl) (e.g., R^{Vb′} is optionally substituted alkyl, optionally substituted alkenyl, or optionally substituted aminoalkyl, e.g., substituted with an N-protecting group, such as any described herein, e.g., trifluoroacetyl, or sulfoalkyl);

R^{12a} is H, optionally substituted alkyl, optionally substituted carboxyaminoalkyl, optionally substituted aminoalkyl (e.g., e.g., substituted with an N-protecting group, such as any described herein, e.g., trifluoroacetyl, or sulfoalkyl), optionally substituted aminoalkenyl, or optionally substituted aminoalkynyl; and

R^{12b} is H, optionally substituted alkyl, optionally substituted alkenyl, optionally substituted alkynyl, optionally substituted hydroxyalkyl, optionally substituted hydroxyalkenyl, optionally substituted hydroxyalkynyl, optionally substituted aminoalkyl, optionally substituted aminoalkenyl, optionally substituted aminoalkynyl (e.g., substituted with an N-protecting group, such as any described herein, e.g., trifluoroacetyl, or sulfoalkyl), optionally substituted alkoxycarbonylacyl, optionally substituted alkoxycarbonylalkoxy, optionally substituted alkoxycarbonylalkyl, optionally substituted alkoxycarbonylalkenyl, optionally substituted alkoxycarbonylalkynyl, optionally substituted alkoxycarbonylalkoxy, optionally substituted carboxyalkoxy, optionally substituted carboxyalkyl, or optionally substituted carbamoylalkyl.

In particular embodiments, T¹ is O (oxo), and T² is S (thio) or Se (seleno). In other embodiments, T¹ is S (thio), and T² is O (oxo) or Se (seleno). In some embodiments, R^{Vb′} is H, optionally substituted alkyl, or optionally substituted alkoxy.

In other embodiments, each R^{12a} and R^{12b} is, independently, H, optionally substituted alkyl, optionally substituted alkenyl, optionally substituted alkynyl, or optionally substituted hydroxyalkyl. In particular embodiments, R^{12a} is H. In other embodiments, both R^{12a} and R^{12b} are H.

In some embodiments, each R^{Vb′} of R^{12b} is, independently, optionally substituted aminoalkyl (e.g., substituted with an N-protecting group, such as any described herein, e.g., trifluoroacetyl, or sulfoalkyl), optionally substituted aminoalkenyl, optionally substituted aminoalkynyl, or optionally substituted acylaminoalkyl (e.g., substituted with an N-protecting group, such as any described herein, e.g., trifluoroacetyl). In some embodiments, the amino and/or alkyl of the optionally substituted aminoalkyl is substituted with one or more of optionally substituted alkyl, optionally substituted alkenyl, optionally substituted sulfoalkyl, optionally substituted carboxy (e.g., substituted with an O-protecting group), optionally substituted hydroxy (e.g., substituted with an O-protecting group), optionally substituted carboxyalkyl (e.g., substituted with an O-protecting group), optionally substituted alkoxycarbonylalkyl (e.g., substituted with an O-protecting group), or N-protecting group. In some embodiments, optionally substituted aminoalkyl is substituted with an optionally substituted sulfoalkyl or optionally substituted alkenyl. In particular embodiments, R^{12a} and R^{Vb″} are both H. In particular embodiments, T¹ is O (oxo), and T² is S (thio) or Se (seleno).

In some embodiments, R^{Vb′} is optionally substituted alkoxycarbonylalkyl or optionally substituted carbamoylalkyl.

In particular embodiments, the optional substituent for R^{12a}, R^{12b}, R^{12c}, or R^{Va} is a polyethylene glycol group (e.g., —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl); or an amino-polyethylene glycol group (e.g., —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl).

In some embodiments, B is a modified cytosine. Exemplary modified cytosines include compounds of Formula (b10)-(b14):

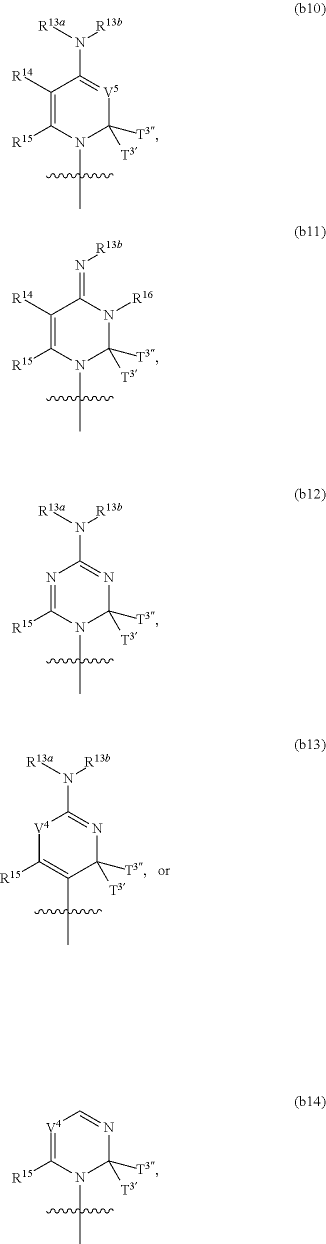

or a pharmaceutically acceptable salt or stereoisomer thereof, wherein

each of T^{3′} and T^{3″} is, independently, H, optionally substituted alkyl, optionally substituted alkoxy, or optionally substituted thioalkoxy, or the combination of T^{3′} and T^{3″} join together (e.g., as in T³) to form O (oxo), S (thio), or Se (seleno);

each V⁴ is, independently, O, S, N(R^{Vc})ₙᵥ, or C(R^{Vc})ₙᵥ, wherein nv is an integer from 0 to 2 and each R^{Vc} is, independently, H, halo, optionally substituted amino acid, optionally substituted alkyl, optionally substituted alkenyl, optionally substituted alkynyl, optionally substituted alkoxy, optionally substituted alkenyloxy, optionally substituted heterocyclyl, optionally substituted alkheterocyclyl, or optionally substituted alkynyloxy (e.g., optionally substituted with any substituent described herein, such as those selected from (1)-(21) for alkyl), wherein the combination of R^{13b} and R^{Vc} can be taken together to form optionally substituted heterocyclyl;

each V⁵ is, independently, N(R^{Vd})ₙᵥ, or C(R^{Vd})ₙᵥ, wherein nv is an integer from 0 to 2 and each R^{Vd} is, independently, H, halo, optionally substituted amino acid, optionally substituted alkyl, optionally substituted alkenyl, optionally substituted alkynyl, optionally substituted alkoxy, optionally substituted alkenyloxy, optionally substituted heterocyclyl, optionally substituted alkheterocyclyl, or optionally substituted alkynyloxy (e.g., optionally substituted with any substituent described herein, such as those selected from (1)-(21) for alkyl) (e.g., V⁵ is —CH or N);

each of R^{13a} and R^{13b} is, independently, H, optionally substituted acyl, optionally substituted acyloxyalkyl, optionally substituted alkyl, or optionally substituted alkoxy, wherein the combination of R^{13b} and R¹⁴ can be taken together to form optionally substituted heterocyclyl;

each R¹⁴ is, independently, H, halo, hydroxy, thiol, optionally substituted acyl, optionally substituted amino acid, optionally substituted alkyl, optionally substituted haloalkyl, optionally substituted alkenyl, optionally substituted alkynyl, optionally substituted hydroxyalkyl (e.g., substituted with an O-protecting group), optionally substituted hydroxyalkenyl, optionally substituted hydroxyalkynyl, optionally substituted alkoxy, optionally substituted alkenyloxy, optionally substituted alkynyloxy, optionally substituted aminoalkoxy, optionally substituted alkoxyalkoxy, optionally substituted acyloxyalkyl, optionally substituted amino (e.g., —NHR, wherein R is H, alkyl, aryl, or phosphoryl), azido, optionally substituted aryl, optionally substituted heterocyclyl, optionally substituted alkheterocyclyl, optionally substituted aminoalkyl, optionally substituted aminoalkenyl, or optionally substituted aminoalkynyl; and

each of R¹⁵ and R¹⁶ is, independently, H, optionally substituted alkyl, optionally substituted alkenyl, or optionally substituted alkynyl.

Further exemplary modified cytosines include those having Formula (b32)-(b35):

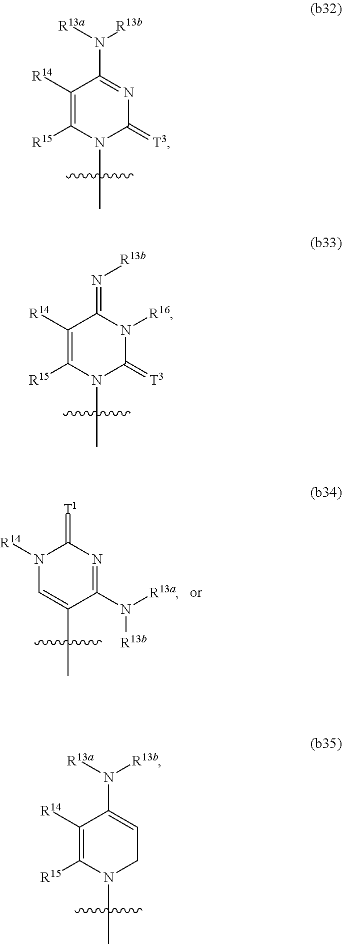

or a pharmaceutically acceptable salt or stereoisomer thereof, wherein

each of T¹ and T³ is, independently, O (oxo), S (thio), or Se (seleno);

each of R^{13a} and R^{13b} is, independently, H, optionally substituted acyl, optionally substituted acyloxyalkyl, optionally substituted alkyl, or optionally substituted alkoxy, wherein the combination of R^{13b} and R¹⁴ can be taken together to form optionally substituted heterocyclyl;

each R¹⁴ is, independently, H, halo, hydroxy, thiol, optionally substituted acyl, optionally substituted amino acid, optionally substituted alkyl, optionally substituted haloalkyl, optionally substituted alkenyl, optionally substituted alkynyl, optionally substituted hydroxyalkyl (e.g., substituted with an O-protecting group), optionally substituted hydroxyalkenyl, optionally substituted hydroxyalkynyl, optionally substituted alkoxy, optionally substituted alkenyloxy, optionally substituted alkynyloxy, optionally substituted aminoalkoxy, optionally substituted alkoxyalkoxy, optionally substituted acyloxyalkyl, optionally substituted amino (e.g., —NHR, wherein R is H, alkyl, aryl, or phosphoryl), azido, optionally substituted aryl, optionally substituted heterocyclyl, optionally substituted alkheterocyclyl, optionally substituted aminoalkyl (e.g., hydroxyalkyl, alkyl, alkenyl, or alkynyl), optionally substituted aminoalkenyl, or optionally substituted aminoalkynyl; and

each of R¹⁵ and R¹⁶ is, independently, H, optionally substituted alkyl, optionally substituted alkenyl, or optionally substituted alkynyl (e.g., R¹⁵ is H, and R¹⁶ is H or optionally substituted alkyl).

In some embodiments, R¹⁵ is H, and R¹⁶ is H or optionally substituted alkyl. In particular embodiments, R¹⁴ is H, acyl, or hydroxyalkyl. In some embodiments, R¹⁴ is halo. In some embodiments, both R¹⁴ and R¹⁵ are H. In some embodiments, both R¹⁵ and R¹⁶ are H. In some embodiments, each of R¹⁴ and R¹⁵ and R¹⁶ is H. In further embodiments, each of R^{13a} and R^{13b} is independently, H or optionally substituted alkyl.

Further non-limiting examples of modified cytosines include compounds of Formula (b36):

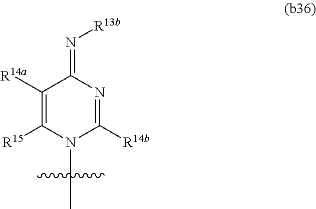

or a pharmaceutically acceptable salt or stereoisomer thereof, wherein

each R^{13b} is, independently, H, optionally substituted acyl, optionally substituted acyloxyalkyl, optionally substituted alkyl, or optionally substituted alkoxy, wherein the combination of R^{13b} and R^{14b} can be taken together to form optionally substituted heterocyclyl;

each R^{14a} and R^{14b} is, independently, H, halo, hydroxy, thiol, optionally substituted acyl, optionally substituted amino acid, optionally substituted alkyl, optionally substituted haloalkyl, optionally substituted alkenyl, optionally substituted alkynyl, optionally substituted hydroxyalkyl (e.g., substituted with an O-protecting group), optionally substituted hydroxyalkenyl, optionally substituted alkoxy, optionally substituted alkenyloxy, optionally substituted alkynyloxy, optionally substituted aminoalkoxy, optionally substituted alkoxyalkoxy, optionally substituted acyloxyalkyl, optionally substituted amino (e.g., —NHR, wherein R is H, alkyl, aryl, phosphoryl, optionally substituted aminoalkyl, or optionally substituted carboxyaminoalkyl), azido, optionally substituted aryl, optionally substituted heterocyclyl, optionally substituted alkheterocyclyl, optionally substituted aminoalkyl, optionally substituted aminoalkenyl, or optionally substituted aminoalkynyl; and

each of R¹⁵ is, independently, H, optionally substituted alkyl, optionally substituted alkenyl, or optionally substituted alkynyl.

In particular embodiments, R^{14b} is an optionally substituted amino acid (e.g., optionally substituted lysine). In some embodiments, R^{14a} is H.

In some embodiments, B is a modified guanine. Exemplary modified guanines include compounds of Formula (b15)-(b17):

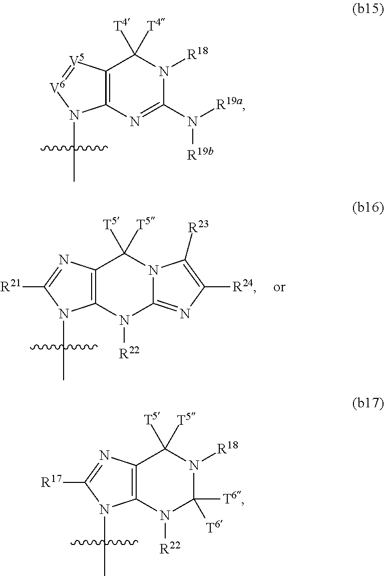

or a pharmaceutically acceptable salt or stereoisomer thereof, wherein

Each of T^{4′}, T^{4″}, T^{5′}, T^{5″}, T^{6′}, and T^{6″} is, independently, H, optionally substituted alkyl, or optionally substituted alkoxy, and wherein the combination of T^{4′} and T^{4″} (e.g., as in T⁴) or the combination of T^{5′} and T^{5″} (e.g., as in T⁵) or the combination of T^{6′} and T^{6″} join together (e.g., as in T⁶) form O (oxo), S (thio), or Se (seleno);

each of V⁵ and V⁶ is, independently, O, S, N(R^{Vd})ₙᵥ, or C(R^{Vd})ₙᵥ, wherein nv is an integer from 0 to 2 and each R^{Vd} is, independently, H, halo, thiol, optionally substituted amino acid, cyano, amidine, optionally substituted aminoalkyl, optionally substituted aminoalkenyl, optionally substituted aminoalkynyl, optionally substituted alkyl, optionally substituted alkenyl, optionally substituted alkynyl, optionally substituted alkoxy, optionally substituted alkenyloxy, optionally substituted alkynyloxy (e.g., optionally substituted with any substituent described herein, such as those selected from (1)-(21) for alkyl), optionally substituted thioalkoxy, or optionally substituted amino; and

each of R¹⁷, R¹⁸, R^{19a}, R^{19b}, R²¹, R²², R²³, and R²⁴ is independently, H, halo, thiol, optionally substituted alkyl, optionally substituted alkenyl, optionally substituted alkynyl, optionally substituted thioalkoxy, optionally substituted amino, or optionally substituted amino acid.

Exemplary modified guanosines include compounds of Formula (b37)-(b40):

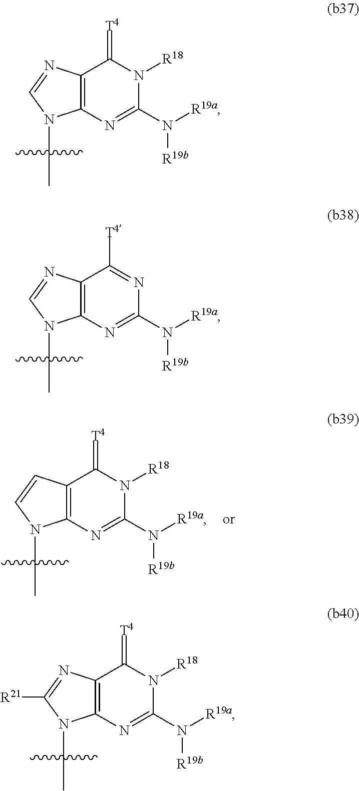

or a pharmaceutically acceptable salt or stereoisomer thereof, wherein

each of T^{4′} is, independently, H, optionally substituted alkyl, or optionally substituted alkoxy, and each T⁴ is, independently, O (oxo), S (thio), or Se (seleno);

each of R¹⁸, R^{19a}, R^{19b}, and R²¹ is, independently, H, halo, thiol, optionally substituted alkyl, optionally substituted alkenyl, optionally substituted alkynyl, optionally substituted thioalkoxy, optionally substituted amino, or optionally substituted amino acid.

In some embodiments, R¹⁸ is H or optionally substituted alkyl. In further embodiments, T⁴ is oxo. In some embodiments, each of R^{19a} and R^{19b} is, independently, H or optionally substituted alkyl.

In some embodiments, B is a modified adenine. Exemplary modified adenines include compounds of Formula (b18)-(b20):

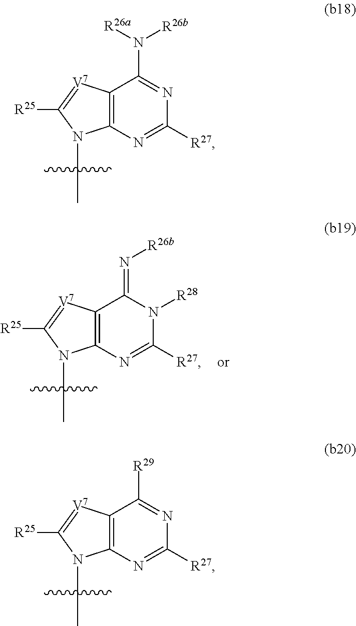

or a pharmaceutically acceptable salt or stereoisomer thereof, wherein

each V⁷ is, independently, O, S, N(R^{Ve})ₙᵥ, or C(R^{Ve})ₙᵥ, wherein nv is an integer from 0 to 2 and each R^{Ve} is, independently, H, halo, optionally substituted amino acid, optionally substituted alkyl, optionally substituted alkenyl, optionally substituted alkynyl, optionally substituted alkoxy, optionally substituted alkenyloxy, or optionally substituted alkynyloxy (e.g., optionally substituted with any substituent described herein, such as those selected from (1)-(21) for alkyl);

each R²⁵ is, independently, H, halo, thiol, optionally substituted alkyl, optionally substituted alkenyl, optionally substituted alkynyl, optionally substituted thioalkoxy, or optionally substituted amino;

each of R^{26a} and R^{26b} is, independently, H, optionally substituted acyl, optionally substituted amino acid, optionally substituted carbamoylalkyl, optionally substituted alkyl, optionally substituted alkenyl, optionally substituted alkynyl, optionally substituted hydroxyalkyl, optionally substituted hydroxyalkenyl, optionally substituted hydroxyalkynyl, optionally substituted alkoxy, or polyethylene glycol group (e.g., —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl); or an amino-polyethylene glycol group (e.g., —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl);

each R²⁷ is, independently, H, optionally substituted alkyl, optionally substituted alkenyl, optionally substituted alkynyl, optionally substituted alkoxy, optionally substituted thioalkoxy, or optionally substituted amino;

each R²⁸ is, independently, H, optionally substituted alkyl, optionally substituted alkenyl, or optionally substituted alkynyl; and

each R²⁹ is, independently, H, optionally substituted acyl, optionally substituted amino acid, optionally substituted carbamoylalkyl, optionally substituted alkyl, optionally substituted alkenyl, optionally substituted alkynyl, optionally substituted hydroxyalkyl, optionally substituted hydroxyalkenyl, optionally substituted alkoxy, or optionally substituted amino.

Exemplary modified adenines include compounds of Formula (b41)-(b43):

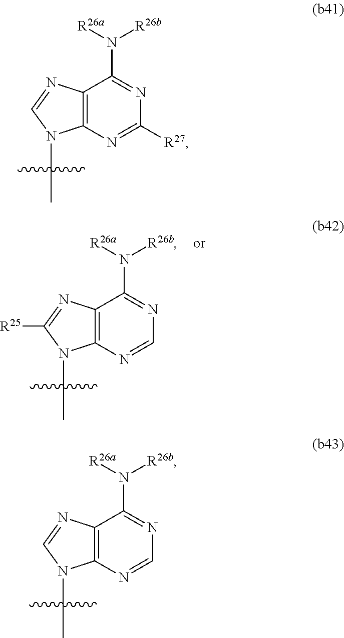

or a pharmaceutically acceptable salt or stereoisomer thereof, wherein

each R²⁵ is, independently, H, halo, thiol, optionally substituted alkyl, optionally substituted alkenyl, optionally substituted alkynyl, optionally substituted thioalkoxy, or optionally substituted amino;

each of R^{26a} and R^{26b} is, independently, H, optionally substituted acyl, optionally substituted amino acid, optionally substituted carbamoylalkyl, optionally substituted alkyl, optionally substituted alkenyl, optionally substituted alkynyl, optionally substituted hydroxyalkyl, optionally substituted hydroxyalkenyl, optionally substituted hydroxyalkynyl, optionally substituted alkoxy, or polyethylene glycol group (e.g., —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl); or an amino-polyethylene glycol group (e.g., —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl); and

each R²⁷ is, independently, H, optionally substituted alkyl, optionally substituted alkenyl, optionally substituted alkynyl, optionally substituted alkoxy, optionally substituted thioalkoxy, or optionally substituted amino.

In some embodiments, R^{26a} is H, and R^{26b} is optionally substituted alkyl. In some embodiments, each of R^{26a} and R^{26b} is, independently, optionally substituted alkyl. In particular embodiments, R²⁷ is optionally substituted alkyl, optionally substituted alkoxy, or optionally substituted thioalkoxy. In other embodiments, R²⁵ is optionally substituted alkyl, optionally substituted alkoxy, or optionally substituted thioalkoxy.

In particular embodiments, the optional substituent for R^{26a}, R^{26b}, or R²⁹ is a polyethylene glycol group (e.g., —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl); or an amino-polyethylene glycol group H (e.g., —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl).

In some embodiments, B may have Formula (b21):

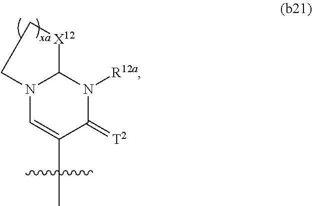

wherein X¹² is, independently, O, S, optionally substituted alkylene (e.g., methylene), or optionally substituted heteroalkylene, xa is an integer from 0 to 3, and R^{12a} and T² are as described herein.

In some embodiments, B may have Formula (b22):

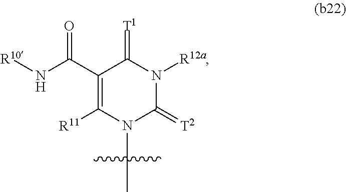

wherein R^{10′} is, independently, optionally substituted alkyl, optionally substituted alkenyl, optionally substituted alkynyl, optionally substituted aryl, optionally substituted heterocyclyl, optionally substituted aminoalkyl, optionally substituted aminoalkenyl, optionally substituted aminoalkynyl, optionally substituted alkoxy, optionally substituted alkoxycarbonylalkyl, optionally substituted alkoxycarbonylalkenyl, optionally substituted alkoxycarbonylalkynyl, optionally substituted alkoxycarbonylalkoxy, optionally substituted carboxyalkoxy, optionally substituted carboxyalkyl, or optionally substituted carbamoylalkyl, and R¹¹, R^{12a}, T¹, and T² are as described herein.

In some embodiments, B may have Formula (b23):

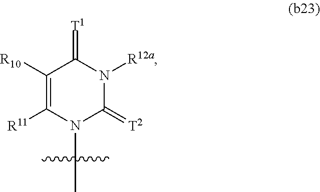

wherein R¹⁰ is optionally substituted heterocyclyl (e.g., optionally substituted furyl, optionally substituted thienyl, or optionally substituted pyrrolyl), optionally substituted aryl (e.g., optionally substituted phenyl or optionally substituted naphthyl), or any substituent described herein (e.g., for R¹⁰); and wherein R¹¹ (e.g., H or any substituent described herein), R^{12a} (e.g., H or any substituent described herein), T¹ (e.g., oxo or any substituent described herein), and T² (e.g., oxo or any substituent described herein) are as described herein.

In some embodiments, B may have Formula (b24):

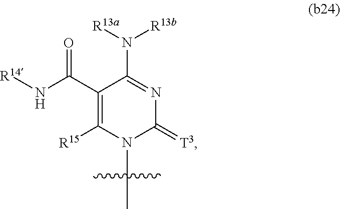

wherein R^{14′} is, independently, optionally substituted alkyl, optionally substituted alkenyl, optionally substituted alkynyl, optionally substituted aryl, optionally substituted heterocyclyl, optionally substituted alkaryl, optionally substituted alkheterocyclyl, optionally substituted aminoalkyl, optionally substituted aminoalkenyl, optionally substituted aminoalkynyl, optionally substituted alkoxy, optionally substituted alkoxycarbonylalkyl, optionally substituted alkoxycarbonylalkenyl, optionally substituted alkoxycarbonylalkynyl, optionally substituted alkoxycarbonylalkoxy, optionally substituted carboxyalkoxy, optionally substituted carboxyalkyl, or optionally substituted carbamoylalkyl, and R^{13a}, R^{13b}, R¹⁵, and T³ are as described herein.

In some embodiments, B may have Formula (b25):

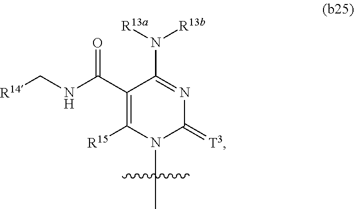

wherein R^{14′} is optionally substituted heterocyclyl (e.g., optionally substituted furyl, optionally substituted thienyl, or optionally substituted pyrrolyl), optionally substituted aryl (e.g., optionally substituted phenyl or optionally substituted naphthyl), or any substituent described herein (e.g., for R¹⁴ or R^{14′}); and wherein R^{13a} (e.g., H or any substituent described herein), R^{13b} (e.g., H or any substituent described herein), R¹⁵ (e.g., H or any substituent described herein), and T³ (e.g., oxo or any substituent described herein) are as described herein.

In some embodiments, B is a nucleobase selected from the group consisting of cytosine, guanine, adenine, and uracil. In some embodiments, B may be:

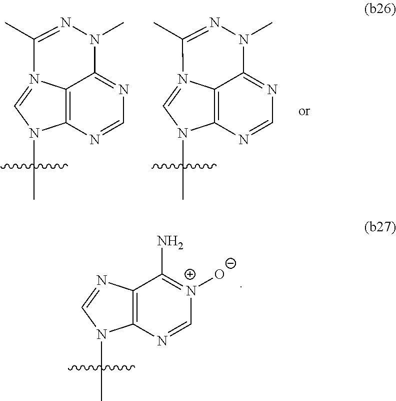

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thiouridine (s²U), 4-thio-uridine (s⁴U), 4-thio-pseudouridine, 2-thio-pseudouridine, 5-hydroxyuridine (ho⁵U), 5-aminoallyl-uridine, 5-halo-uridine (e.g., 5-iodo-uridineor 5-bromo-uridine), 3-methyluridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyluridine (τm⁵U), 1-taurinomethyl-pseudouridine, 5-taurinomethyl-2-thio-uridine (τm⁵s²U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formylcytidine (f⁵C), N4-methylcytidine (m⁴C), 5-methyl-cytidine (m⁵C), 5-halo-cytidine (e.g., 5-iodo-cytidine), 5-hydroxymethylcytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴2Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-aminopurine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyladenosine (m¹A), 2-methyl-adenine (m²A), N6-methyladenosine (m⁶A), 2-methylthio-N6-methyl-adenosine (ms² m⁶A), N6-isopentenyladenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyladenosine (g⁶A), N6-threonylcarbamoyladenosine (t⁶A), N6-methyl-N6-threonylcarbamoyl-adenosine (m⁶t⁶A), 2-methylthio-N6-threonyl carbamoyladenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyladenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶2Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OHyW), undermodified hydroxywybutosine (OHyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methylguanosine (m⁷G), 6-thio-7-methyl-guanosine, 7-methyl-inosine, 6-methoxy-guanosine, 1-methylguanosine (m¹G), N2-methyl-guanosine (m²G), N2,N2-dimethyl-guanosine (m²2G), N2,7-dimethyl-guanosine (m^{2,7}G), N2,N2,7-dimethyl-guanosinem (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m¹Gm), N2,7-dimethyl-2′-O-methyl-guanosine (m^{2,7}Gm), 2′-O-methyl-inosine (Im), 1,2′-O-dimethyl-inosine (m¹Im), 2′-O-ribosylguanosine (phosphate) (Gr(p)), 1-thio-guanosine, O6-methyl-guanosine, T-F-ara-guanosine, and 2′-F-guanosine.

In some embodiments, a modified nucleotide is 5′-O-(1-Thiophosphate)-Adenosine, 5′-O-(1-Thiophosphate)-Cytidine, 5′-O-(1-Thiophosphate)-Guanosine, 5′-O-(1-Thiophosphate)-Uridine or 5′-O-(1-Thiophosphate)-Pseudouridine.

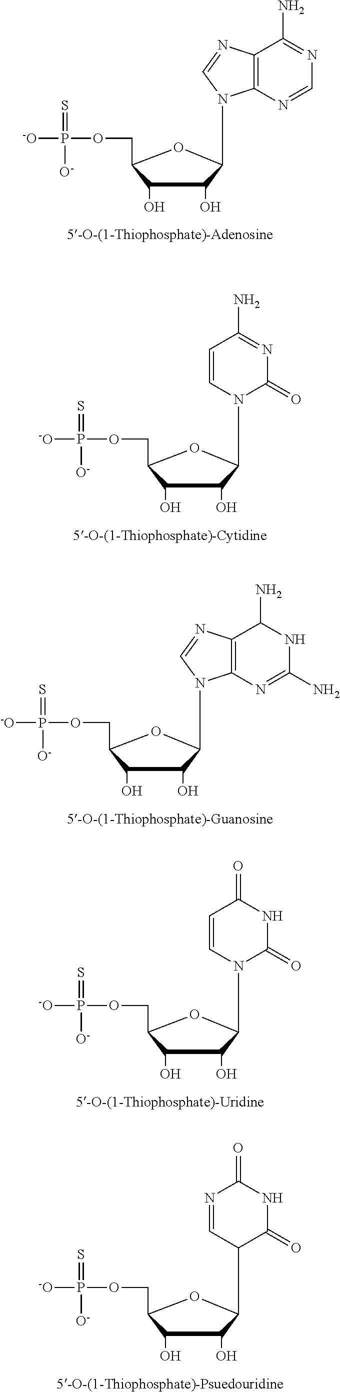

The α-thio substituted phosphate moiety is provided to confer stability to RNA and DNA polymers through the unnatural phosphorothioate backbone linkages.

Phosphorothioate DNA and RNA have increased nuclease resistance and subsequently a longer half-life in a cellular environment. Phosphorothioate linked nucleic acids are expected to also reduce the innate immune response through weaker binding/activation of cellular innate immune molecules.

The nucleobase of the nucleotide can be independently selected from a purine, a pyrimidine, a purine or pyrimidine analog. For example, the nucleobase can each be independently selected from adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

In some embodiments, the modified nucleotide is a compound of Formula XI:

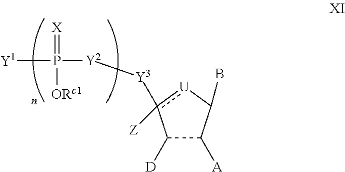

wherein:

[symbol] denotes a single or a double bond;

- - - denotes an optional single bond;

U is O, S, —NR^{a}—, or —CR^{a}R^{b}— when [symbol] denotes a single bond, or U is —CR^{a}— when [symbol] denotes a double bond;

Z is H, C₁₋₁₂ alkyl, or C₆₋₂₀ aryl, or Z is absent when [symbol] denotes a double bond; and

Z can be —CR^{a}R^{b}— and form a bond with A;

A is H, OH, NHR wherein R═ alkyl or aryl or phosphoryl, sulfate, —NH₂, N₃, azido, —SH, N an amino acid, or a peptide comprising 1 to 12 amino acids;

D is H, OH, NHR wherein R═ alkyl or aryl or phosphoryl, —NH₂, —SH, an amino acid, a peptide comprising 1 to 12 amino acids, or a group of Formula XII:

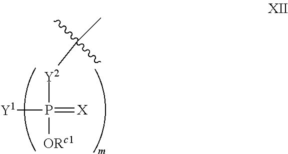

or A and D together with the carbon atoms to which they are attached form a 5-membered ring;

X is O or S;

each of Y¹ is independently selected from —OR^{a1}, —NR^{a1}R^{b1}, and —SR^{a1};

each of Y² and Y³ are independently selected from O, —CR^{a}R^{b}—, S or a linker comprising one or more atoms selected from the group consisting of C, O, N, and S;

n is 0, 1, 2, or 3;

m is 0, 1, 2 or 3;

B is nucleobase;

R^{a} and R^{b} are each independently H, C₁₋₁₂ alkyl, C₂₋₁₂ alkenyl, C₂₋₁₂ alkynyl, or C₆₋₂₀ aryl;

R^{c} is H, C₁₋₁₂ alkyl, C₂₋₁₂ alkenyl, phenyl, benzyl, a polyethylene glycol group, or an amino-polyethylene glycol group;

R^{a1} and R^{b1} are each independently H or a counterion; and

—OR^{c1} is OH at a pH of about 1 or —OR^{c1} is O⁻ at physiological pH;

provided that the ring encompassing the variables A, B, D, U, Z, Y² and Y³ cannot be ribose.

In some embodiments, B is a nucleobase selected from the group consisting of cytosine, guanine, adenine, and uracil.

In some embodiments, the nucleobase is a pyrimidine or derivative thereof.

In some embodiments, the modified nucleotides are a compound of Formula XI-a:

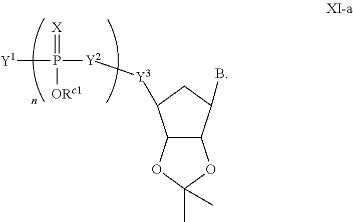

In some embodiments, the modified nucleotides are a compound of Formula XI-b:

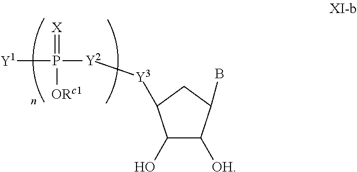

In some embodiments, the modified nucleotides are a compound of Formula XI-c1, XI-c2, or XI-c3:

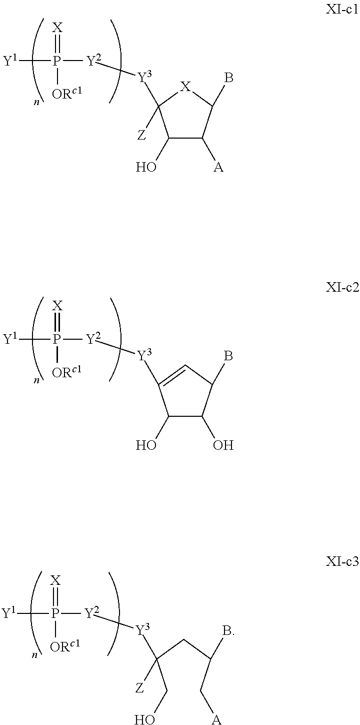

In some embodiments, the modified nucleotides are a compound of Formula XI:

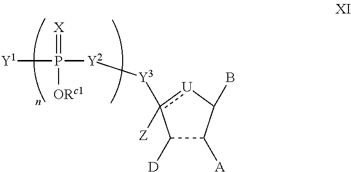

wherein:

[symbol] denotes a single or a double bond;

- - - denotes an optional single bond;

U is O, S, —NR^{a}—, or —CR^{a}R^{b}— when [symbol] denotes a single bond, or U is —CR^{a}— when [symbol] denotes a double bond;

Z is H, C₁₋₁₂ alkyl, or C₆₋₂₀ aryl, or Z is absent when [symbol] denotes a double bond; and

Z can be —CR^{a}R^{b}— and form a bond with A;

A is H, OH, sulfate, —NH₂, —SH, an amino acid, or a peptide comprising 1 to 12 amino acids;

D is H, OH, —NH₂, —SH, an amino acid, a peptide comprising 1 to 12 amino acids, or a group of Formula XII:

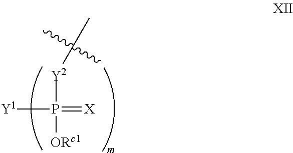

or A and D together with the carbon atoms to which they are attached form a 5-membered ring;

X is O or S;

each of Y¹ is independently selected from —OR^{a1}, —NR^{a1}R^{b1} and —SR^{a1};

each of Y² and Y³ are independently selected from O, —CR^{a}R^{b}—, S or a linker comprising one or more atoms selected from the group consisting of C, O, N, and S;

n is 0, 1, 2, or 3;

m is 0, 1, 2 or 3;

B is a nucleobase of Formula XIII:

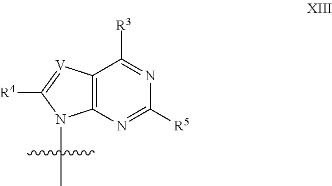

wherein:

V is N or positively charged NR^{c};

R³ is NR^{c}R^{d}, —OR^{a}, or —SR^{a};

R⁴ is H or can optionally form a bond with Y³;

R⁵ is H, —NR^{c}R^{d}, or —OR^{a};

R^{a} and R^{b} are each independently H, C₁₋₁₂ alkyl, C₂₋₁₂ alkenyl, C₂₋₁₂ alkynyl, or C₆₋₂₀ aryl;

R^{c} is H, C₁₋₁₂ alkyl, C₂₋₁₂ alkenyl, phenyl, benzyl, a polyethylene glycol group, or an amino-polyethylene glycol group;

R^{a1} and R^{b1} are each independently H or a counterion; and

—OR^{c1} is OH at a pH of about 1 or —OR^{c1} is O⁻ at physiological pH.

In some embodiments, B is:

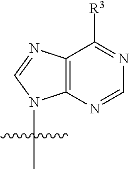

wherein R³ is —OH, —SH, or

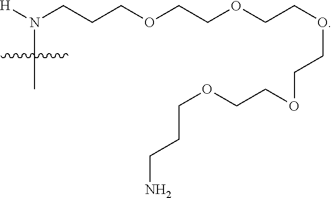 [SMILES: *NCCCOCCOCCOCCOCCOCCCN]

In some embodiments, B is:

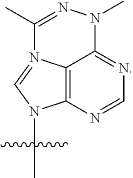

In some embodiments, B is:

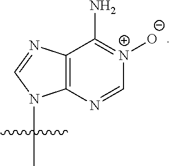 [SMILES: *n1cnc2c(N)[n+]([O-])cnc21]

In some embodiments, the modified nucleotides are a compound of Formula I-d:

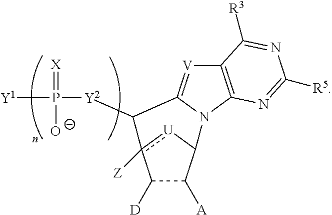

In some embodiments, the modified nucleotides are a compound selected from the group consisting of:

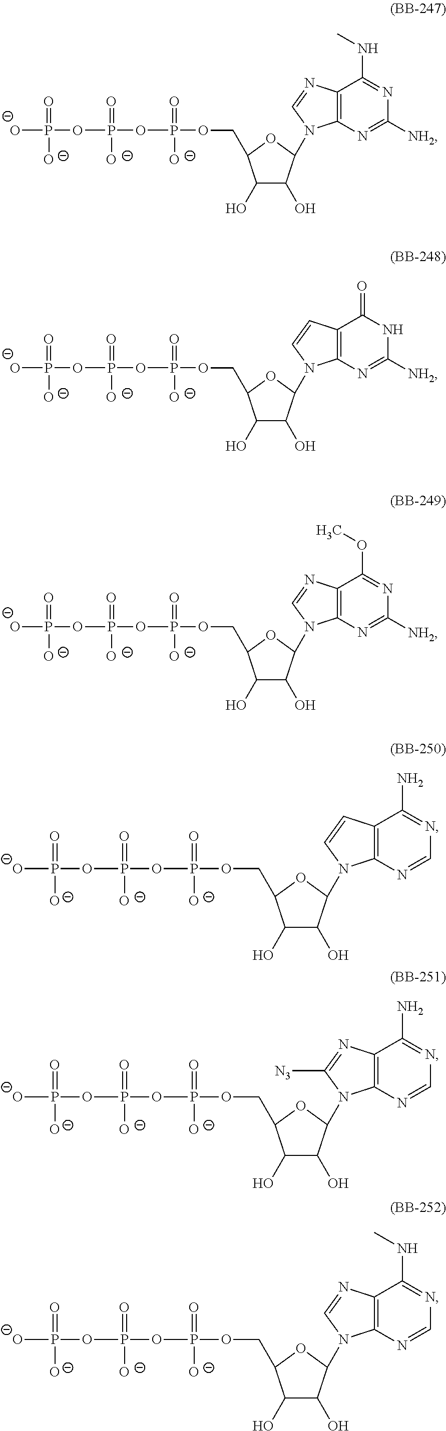 [SMILES: CNc1nc(N)nc2c1ncn2C1OC(COP(=O)([O-])OP(=O)([O-])OP(=O)([O-])[O-])C(O)C1O ; Nc1nc2c(ccn2C2OC(COP(=O)([O-])OP(=O)([O-])OP(=O)([O-])[O-])C(O)C2O)c(=O)[nH]1 ; COc1nc(N)nc2c1ncn2C1OC(COP(=O)([O-])OP(=O)([O-])OP(=O)([O-])[O-])C(O)C1O ; Nc1ncnc2c1ccn2C1OC(COP(=O)([O-])OP(=O)([O-])OP(=O)([O-])[O-])C(O)C1O ; [N-]=[N+]=Nc1nc2c(N)ncnc2n1C1OC(COP(=O)([O-])OP(=O)([O-])OP(=O)([O-])[O-])C(O)C1O ; CNc1ncnc2c1ncn2C1OC(COP(=O)([O-])OP(=O)([O-])OP(=O)([O-])[O-])C(O)C1O]

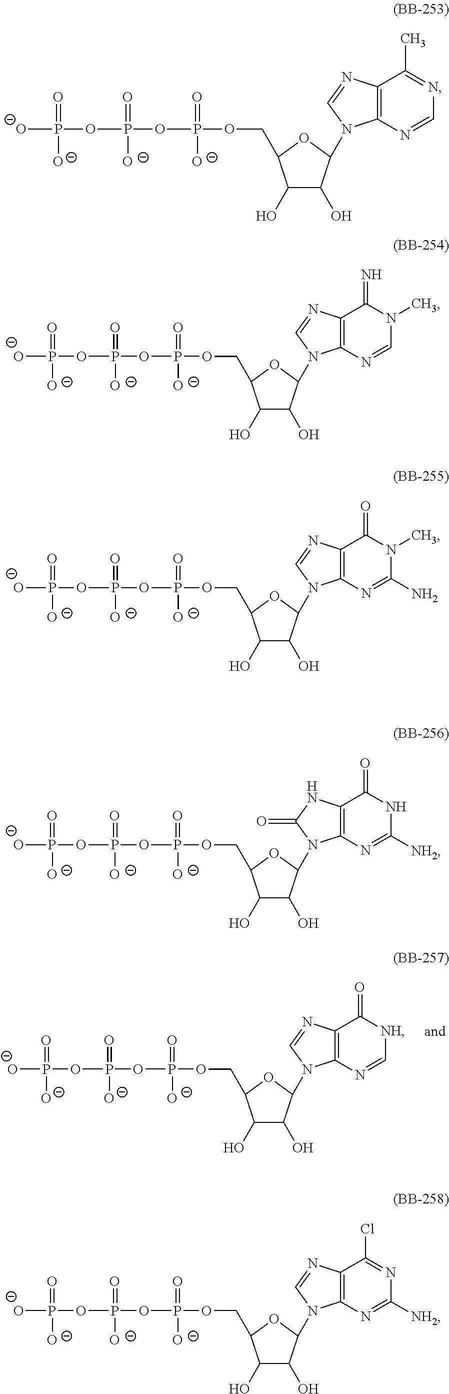

or a pharmaceutically acceptable salt thereof.

In some embodiments, the modified nucleotides are a compound selected from the group consisting of:

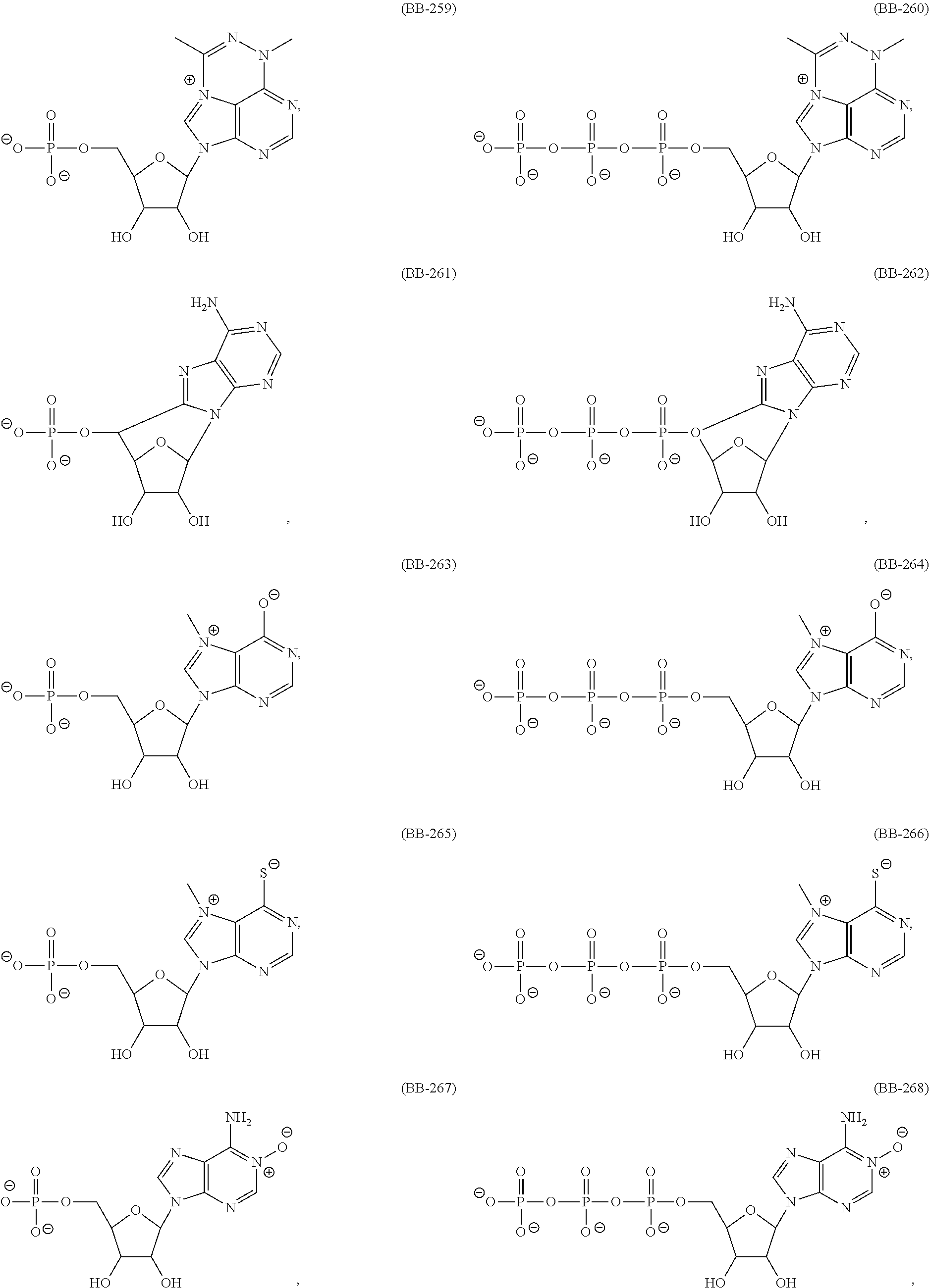

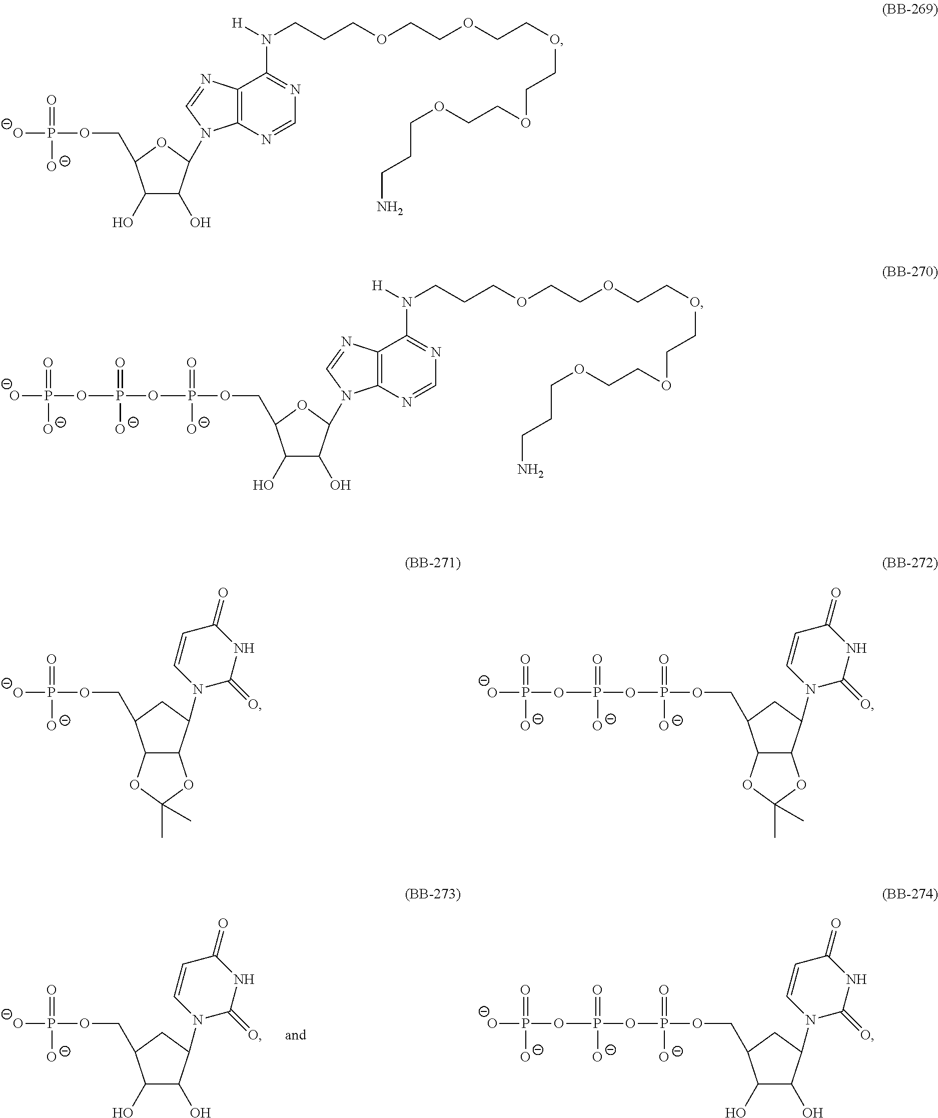

or a pharmaceutically acceptable salt thereof.

### Modifications on the Internucleoside Linkage

The modified nucleotides, which may be incorporated into a nucleic acid or modified RNA molecule, can be modified on the internucleoside linkage (e.g., phosphate backbone). Herein, in the context of the nucleic acids or modified RNA backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. While not wishing to be bound by theory, phosphorothioate linked nucleic acids or modified RNA molecules are expected to also reduce the innate immune response through weaker binding/activation of cellular innate immune molecules.

In specific embodiments, a modified nucleoside includes an alpha-thio-nucleoside (e.g., 5′-O-(1-thiophosphate)-adenosine, 5′-O-(1-thiophosphate)-cytidine (α-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein below.

### Combinations of Modified Sugars, Nucleobases, and Internucleoside Linkages

The nucleic acids or modified RNA of the invention can include a combination of modifications to the sugar, the nucleobase, and/or the internucleoside linkage. These combinations can include any one or more modifications described herein. For examples, any of the nucleotides described herein in Formulas (Ia), (Ia-1)-(Ia-3), (Ib)-(If), (IIa)-(IIp), (IIb-1), (IIb-2), (IIc-1)-(IIc-2), (IIn-1), (IIn-2), (IVa)-(IV1), and (IXa)-(IXr) can be combined with any of the nucleobases described herein (e.g., in Formulas (b1)-(b43) or any other described herein).

Further examples of modified nucleotides and modified nucleotide combinations are provided below in Table 3. These combinations of modified nucleotides can be used to form the nucleic acids or modified RNA of the invention. Unless otherwise noted, the modified nucleotides may be completely substituted for the natural nucleotides of the nucleic acids or modified RNA of the invention. As a non-limiting example, the natural nucleotide uridine may be substituted with a modified nucleoside described herein. In another non-limiting example, the natural nucleotide uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9%) with at least one of the modified nucleoside disclosed herein.

TABLE 3

| Modified Nucleotide | Modified Nucleotide Combination |
|---|---|
| 6-aza-cytidine | α-thio-cytidine/5-iodo-uridine |
| 2-thio-cytidine | α-thio-cytidine/N1-methyl-pseudo-uridine |
| α-thio-cytidine | α-thio-cytidine/α-thio-uridine |
| Pseudo-iso-cytidine | α-thio-cytidine/5-methyl-uridine |
| 5-aminoallyl-uridine | α-thio-cytidine/pseudo-uridine |
| 5-iodo-uridine | Pseudo-iso-cytidine/5-iodo-uridine |
| N1-methyl-pseudouridine | Pseudo-iso-cytidine/N1-methyl-pseudo-uridine |
| 5,6-dihydrouridine | Pseudo-iso-cytidine/α-thio-uridine |
| α-thio-uridine | Pseudo-iso-cytidine/5-methyl-uridine |
| 4-thio-uridine | Pseudo-iso-cytidine/Pseudo-uridine |
| 6-aza-uridine | Pyrrolo-cytidine/5-iodo-uridine |
| 5-hydroxy-uridine | Pyrrolo-cytidine/N1-methyl-pseudo-uridine |
| Deoxy-thymidine | Pyrrolo-cytidine/α-thio-uridine |
| Pseudo-uridine | Pyrrolo-cytidine/5-methyl-uridine |
| Inosine | Pyrrolo-cytidine/Pseudo-uridine |
| α-thio-guanosine | 5-methyl-cytidine/5-iodo-uridine |
| 8-oxo-guanosine | 5-methyl-cytidine/N1-methyl-pseudo-uridine |
| O6-methyl-guanosine | 5-methyl-cytidine/α-thio-uridine |
| 7-deaza-guanosine | 5-methyl-cytidine/5-methyl-uridine |
| No modification | 5-methyl-cytidine/Pseudo-uridine |
| N1-methyl-adenosine | about 25% of cytosines are Pseudo-iso-cytidine |
| 2-amino-6-Chloro-purine | about 25% of uridines are N1-methyl-pseudo-uridine |
| N6-methyl-2-amino-purine | 25% N1-Methyl-pseudo-uridine/75%-pseudo-uridine |
| 6-Chloro-purine | about 50% of the cytosines are pyrrolo-cytidine |
| N6-methyl-adenosine | 5-methyl-cytidine/5-iodo-uridine |
| α-thio-adenosine | 5-methyl-cytidine/N1-methyl-pseudouridine |
| 8-azido-adenosine | 5-methyl-cytidine/α-thio-uridine |
| 7-deaza-adenosine | 5-methyl-cytidine/5-methyl-uridine |
| Pyrrolo-cytidine | 5-methyl-cytidine/pseudouridine |
| 5-methyl-cytidine | about 25% of cytosines are 5-methyl-cytidine |
| N4-acetyl-cytidine | about 50% of cytosines are 5-methyl-cytidine |
| 5-methyl-uridine | 5-methyl-cytidine/5-methoxy-uridine |
| 5-iodo-cytidine | 5-methyl-cytidine/5-bromo-uridine |
| | 5-methyl-cytidine/2-thio-uridine |
| | 5-methyl-cytidine/about 50% of uridines are 2-thio- |
| | uridine |
| | about 50% of uridines are 5-methyl-cytidine/about 50% |
| | of uridines are 2-thio-uridine |
| | N4-acetyl-cytidine/5-iodo-uridine |
| | N4-acetyl-cytidine/N1-methyl-pseudouridine |
| | N4-acetyl-cytidine/α-thio-uridine |
| | N4-acetyl-cytidine/5-methyl-uridine |
| | N4-acetyl-cytidine/pseudouridine |
| | about 50% of cytosines are N4-acetyl-cytidine |
| | about 25% of cytosines are N4-acetyl-cytidine |
| | N4-acetyl-cytidine/5-methoxy-uridine |
| | N4-acetyl-cytidine/5-bromo-uridine |
| | N4-acetyl-cytidine/2-thio-uridine |
| | about 50% of cytosines are N4-acetyl-cytidine/about 50% |
| | of uridines are 2-thio-uridine |
| | pseudoisocytidine/about 50% of uridines are N1-methyl- |
| | pseudouridine and about 50% of uridines are |
| | pseudouridine |
| | pseudoisocytidine/about 25% of uridines are N1-methyl- |
| | pseudouridine and about 25% of uridines are |
| | pseudouridine |
| | (e.g., 25% N1-methyl-pseudouridine/75% pseudouridine) |
| | about 50% of the cytosines are α-thio-cytidine |

Certain modified nucleotides and nucleotide combinations have been explored by the current inventors. These findings are described in U.S. Provisional Application No. 61/404,413, filed on Oct. 1, 2010, entitled Engineered Nucleic Acids and Methods of Use Thereof, U.S. patent application Ser. No. 13/251,840, filed on Oct. 3, 2011, entitled Modified Nucleotides, and Nucleic Acids, and Uses Thereof, now abandoned, U.S. patent application Ser. No. 13/481,127, filed on May 25, 2012, entitled Modified Nucleotides, and Nucleic Acids, and Uses Thereof, International Patent Publication No WO2012045075, filed on Oct. 3, 2011, entitled Modified Nucleosides, Nucleotides, And Nucleic Acids, and Uses Thereof, U.S. Patent Publication No US20120237975 filed on Oct. 3, 2011, entitled Engineered Nucleic Acids and Method of Use Thereof, and International Patent Publication No WO2012045082, which are incorporated by reference in their entireties.

Further examples of modified nucleotide combinations are provided below in Table 4. These combinations of modified nucleotides can be used to form the nucleic acids of the invention.

TABLE 4

| Modified Nucleotide | Modified Nucleotide Combination |
|---|---|
| modified cytidine having one or more | modified cytidine with (b10)/pseudouridine |
| nucleobases of Formula (b10) | modified cytidine with (b10)/N1-methyl-pseudouridine |
| | modified cytidine with (b10)/5-methoxy-uridine |
| | modified cytidine with (b10)/5-methyl-uridine |
| | modified cytidine with (b10)/5-bromo-uridine |
| | modified cytidine with (b10)/2-thio-uridine |
| | about 50% of cytidine substituted with modified cytidine |
| | (b10)/about 50% of uridines are 2-thio-uridine |
| modified cytidine having one or more | modified cytidine with (b32)/pseudouridine |
| nucleobases of Formula (b32) | modified cytidine with (b32)/N1-methyl-pseudouridine |
| | modified cytidine with (b32)/5-methoxy-uridine |
| | modified cytidine with (b32)/5-methyl-uridine |
| | modified cytidine with (b32)/5-bromo-uridine |
| | modified cytidine with (b32)/2-thio-uridine |
| | about 50% of cytidine substituted with modified cytidine |
| | (b32)/about 50% of uridines are 2-thio-uridine |
| modified uridine having one or more | modified uridine with (b1)/N4-acetyl-cytidine |
| nucleobases of Formula (b1) | modified uridine with (b1)/5-methyl-cytidine |
| modified uridine having one or more | modified uridine with (b8)/N4-acetyl-cytidine |
| nucleobases of Formula (b8) | modified uridine with (b8)/5-methyl-cytidine |
| modified uridine having one or more | modified uridine with (b28)/N4-acetyl-cytidine |
| nucleobases of Formula (b28) | modified uridine with (b28)/5-methyl-cytidine |
| modified uridine having one or more | modified uridine with (b29)/N4-acetyl-cytidine |
| nucleobases of Formula (b29) | modified uridine with (b29)/5-methyl-cytidine |
| modified uridine having one or more | modified uridine with (b30)/N4-acetyl-cytidine |
| nucleobases of Formula (b30) | modified uridine with (b30)/5-methyl-cytidine |

In some embodiments, at least 25% of the cytosines are replaced by a compound of Formula (b10)-(b14), (b24), (b25), or (b32)-(b35) (e.g., at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of, e.g., a compound of Formula (b10) or (b32)).

In some embodiments, at least 25% of the uracils are replaced by a compound of Formula (b1)-(b9), (b21)-(b23), or (b28)-(b31) (e.g., at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of, e.g., a compound of Formula (b1), (b8), (b28), (b29), or (b30)).

In some embodiments, at least 25% of the cytosines are replaced by a compound of Formula (b10)-(b14), (b24), (b25), or (b32)-(b35) (e.g. Formula (b10) or (b32)), and at least 25% of the uracils are replaced by a compound of Formula (b1)-(b9), (b21)-(b23), or (b28)-(b31) (e.g. Formula (b1), (b8), (b28), (b29), or (b30)) (e.g., at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%).

### Modifications Including Linker and a Payload

The nucleobase of the nucleotide can be covalently linked at any chemically appropriate position to a payload, e.g., detectable agent or therapeutic agent. For example, the nucleobase can be deaza-adenosine or deaza-guanosine and the linker can be attached at the C-7 or C-8 positions of the deaza-adenosine or deaza-guanosine. In other embodiments, the nucleobase can be cytosine or uracil and the linker can be attached to the N-3 or C-5 positions of cytosine or uracil. Scheme 1 below depicts an exemplary modified nucleotide wherein the nucleobase, adenine, is attached to a linker at the C-7 carbon of 7-deaza adenine. In addition, Scheme 1 depicts the modified nucleotide with the linker and payload, e.g., a detectable agent, incorporated onto the 3′ end of the mRNA. Disulfide cleavage and 1,2-addition of the thiol group onto the propargyl ester releases the detectable agent. The remaining structure (depicted, for example, as pApC5Parg in Scheme 1) is the inhibitor. The rationale for the structure of the modified nucleotides is that the tethered inhibitor sterically interferes with the ability of the polymerase to incorporate a second base. Thus, it is critical that the tether be long enough to affect this function and that the inhibitor be in a stereochemical orientation that inhibits or prohibits second and follow on nucleotides into the growing nucleic acid or modified RNA strand.

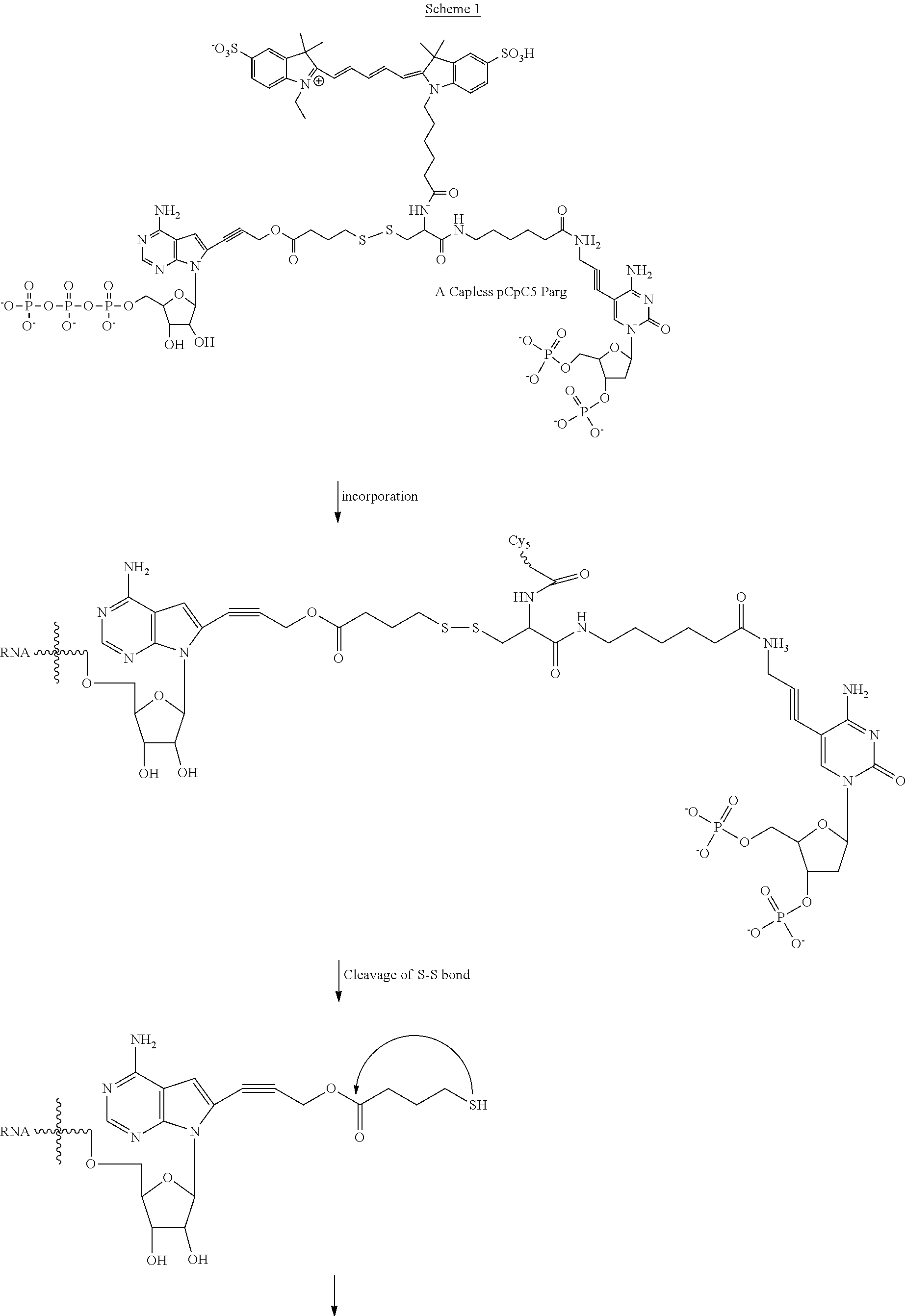

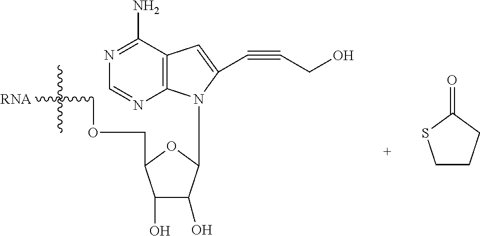

### Linker

The term “linker” as used herein refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., detectable or therapeutic agent, at a second end. The linker is of sufficient length as to not interfere with incorporation into a nucleic acid sequence.

Examples of chemical groups that can be incorporated into the linker include, but are not limited to, an alkyl, alkene, an alkyne, an amido, an ether, a thioether, an or an ester group. The linker chain can also comprise part of a saturated, unsaturated or aromatic ring, including polycyclic and heteroaromatic rings wherein the heteroaromatic ring is an aryl group containing from one to four heteroatoms, N, O or S. Specific examples of linkers include, but are not limited to, unsaturated alkanes, polyethylene glycols, and dextran polymers.

For example, the linker can include ethylene or propylene glycol monomeric units, e.g., diethylene glycol, dipropylene glycol, triethylene glycol, tripropylene glycol, tetraethylene glycol, or tetraethylene glycol. In some embodiments, the linker can include a divalent alkyl, alkenyl, and/or alkynyl moiety. The linker can include an ester, amide, or ether moiety.

Other examples include cleavable moieties within the linker, such as, for example, a disulfide bond (—S—S—) or an azo bond (—N═N—), which can be cleaved using a reducing agent or photolysis. A cleavable bond incorporated into the linker and attached to a modified nucleotide, when cleaved, results in, for example, a short “scar” or chemical modification on the nucleotide. For example, after cleaving, the resulting scar on a nucleotide base, which formed part of the modified nucleotide, and is incorporated into a nucleic acid or modified RNA strand, is unreactive and does not need to be chemically neutralized. This increases the ease with which a subsequent nucleotide can be incorporated during sequencing of a nucleic acid polymer template. For example, conditions include the use of tris(2-carboxyethyl)phosphine (TCEP), dithiothreitol (DTT) and/or other reducing agents for cleavage of a disulfide bond. A selectively severable bond that includes an amido bond can be cleaved for example by the use of TCEP or other reducing agents, and/or photolysis. A selectively severable bond that includes an ester bond can be cleaved for example by acidic or basic hydrolysis.

### Payload

The methods and compositions described herein are useful for delivering a payload to a biological target. The payload can be used, e.g., for labeling (e.g., a detectable agent such as a fluorophore), or for therapeutic purposes (e.g., a cytotoxin or other therapeutic agent).

### Payload: Therapeutic Agents

In some embodiments the payload is a therapeutic agent such as a cytotoxin, radioactive ion, chemotherapeutic, or other therapeutic agent. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Radioactive ions include, but are not limited to iodine (e.g., iodine 125 or iodine 131), strontium 89, phosphorous, palladium, cesium, iridium, phosphate, cobalt, yttrium 90, Samarium 153 and praseodymium. Other therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

### Payload:Detectable Agents

Examples of detectable substances include various organic small molecules, inorganic compounds, nanoparticles, enzymes or enzyme substrates, fluorescent materials, luminescent materials, bioluminescent materials, chemiluminescent materials, radioactive materials, and contrast agents. Such optically-detectable labels include for example, without limitation, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′ 5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]-naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cyanine-3 (Cy3); Cyanine-5 (Cy5); Cyanine-5.5 (Cy5.5), Cyanine-7 (Cy7); IRD 700; IRD 800; Alexa 647; La Jolta Blue; phthalo cyanine; and naphthalo cyanine. In some embodiments, the detectable label is a fluorescent dye, such as Cy5 and Cy3.

Examples luminescent material includes luminol; examples of bioluminescent materials include luciferase, luciferin, and aequorin.

Examples of suitable radioactive material include ¹⁸F, ⁶⁷Ga, ^{81m}Kr, ⁸²Rb, ¹¹¹In, ¹²³I, ¹³³Xe, ²⁰¹Tl, ¹²⁵I, ³⁵S, ¹⁴C, or ³H, ^{99m}Tc (e.g., as pertechnetate (technetate(VII), TcO₄⁻) either directly or indirectly, or other radioisotope detectable by direct counting of radioemission or by scintillation counting.

In addition, contrast agents, e.g., contrast agents for MRI or NMR, for X-ray CT, Raman imaging, optical coherence tomography, absorption imaging, ultrasound imaging, or thermal imaging can be used. Exemplary contrast agents include gold (e.g., gold nanoparticles), gadolinium (e.g., chelated Gd), iron oxides (e.g., superparamagnetic iron oxide (SPIO), monocrystalline iron oxide nanoparticles (MIONs), and ultrasmall superparamagnetic iron oxide (USPIO)), manganese chelates (e.g., Mn-DPDP), barium sulfate, iodinated contrast media (iohexol), microbubbles, or perfluorocarbons can also be used.

In some embodiments, the detectable agent is a non-detectable pre-cursor that becomes detectable upon activation. Examples include fluorogenic tetrazine-fluorophore constructs (e.g., tetrazine-BODIPY FL, tetrazine-Oregon Green 488, or tetrazine-BODIPY TMR-X) or enzyme activatable fluorogenic agents (e.g., PROSENSE (VisEn Medical)).

When the compounds are enzymatically labeled with, for example, horseradish peroxidase, alkaline phosphatase, or luciferase, the enzymatic label is detected by determination of conversion of an appropriate substrate to product.

In vitro assays in which these compositions can be used include enzyme linked immunosorbent assays (ELISAs), immunoprecipitations, immunofluorescence, enzyme immunoassay (EIA), radioimmunoassay (RIA), and Western blot analysis.

Labels other than those described herein are contemplated by the present disclosure, including other optically-detectable labels. Labels can be attached to the modified nucleotide of the present disclosure at any position using standard chemistries such that the label can be removed from the incorporated base upon cleavage of the cleavable linker.

Payload:Cell Penetrating Payloads

In some embodiments, the modified nucleotides and modified nucleic acids can also include a payload that can be a cell penetrating moiety or agent that enhances intracellular delivery of the compositions. For example, the compositions can include a cell-penetrating peptide sequence that facilitates delivery to the intracellular space, e.g., HIV-derived TAT peptide, penetratins, transportans, or hCT derived cell-penetrating peptides, see, e.g., Caron et al., (2001) Mol Ther. 3(3):310-8; Langel, Cell-Penetrating Peptides: Processes and Applications (CRC Press, Boca Raton Fla. 2002); El-Andaloussi et al., (2005) Curr Pharm Des. 11(28):3597-611; and Deshayes et al., (2005) Cell Mol Life Sci. 62(16):1839-49. The compositions can also be formulated to include a cell penetrating agent, e.g., liposomes, which enhance delivery of the compositions to the intracellular space.

### Payload:Biological Targets

The modified nucleotides and modified nucleic acids described herein can be used to deliver a payload to any biological target for which a specific ligand exists or can be generated. The ligand can bind to the biological target either covalently or non-covalently.

Exemplary biological targets include biopolymers, e.g., antibodies, nucleic acids such as RNA and DNA, proteins, enzymes; exemplary proteins include enzymes, receptors, and ion channels. In some embodiments the target is a tissue- or cell-type specific marker, e.g., a protein that is expressed specifically on a selected tissue or cell type. In some embodiments, the target is a receptor, such as, but not limited to, plasma membrane receptors and nuclear receptors; more specific examples include G-protein-coupled receptors, cell pore proteins, transporter proteins, surface-expressed antibodies, HLA proteins, MHC proteins and growth factor receptors.

### Synthesis of Modified Nucleotides

The modified nucleosides and nucleotides disclosed herein can be prepared from readily available starting materials using the following general methods and procedures. It is understood that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

Preparation of modified nucleosides and nucleotides can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents, which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Resolution of racemic mixtures of modified nucleosides and nucleotides can be carried out by any of numerous methods known in the art. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Resolution of racemic mixtures can also be carried out by elution on a column packed with an optically active resolving agent (e.g., dinitrobenzoylphenylglycine). Suitable elution solvent composition can be determined by one skilled in the art.

Exemplary syntheses of modified nucleotides, which are incorporated into nucleic acids or modified RNA, e.g., RNA or mRNA, are provided below in Scheme 2 through Scheme 12. Scheme 2 provides a general method for phosphorylation of nucleosides, including modified nucleosides.

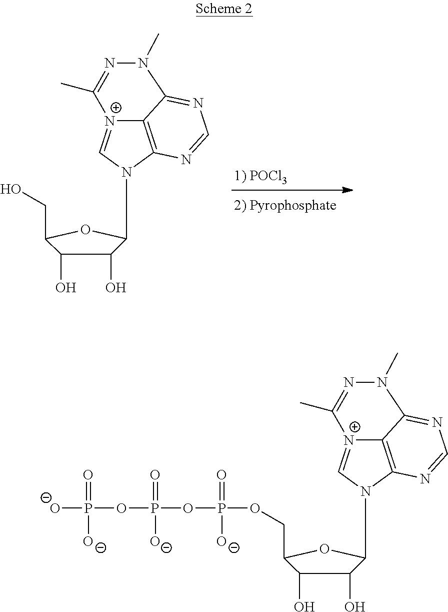 [SMILES: CC1=NN(C)c2ncnc3c2[n+]1cn3CC1OC(CO)C(O)C1O ; CC1=NN(C)c2ncnc3c2[n+]1cn3CC1OC(COP(=O)([O-])OP(=O)([O-])OP(=O)([O-])[O-])C(O)C1O]

Various protecting groups may be used to control the reaction. For example, Scheme 3 provides the use of multiple protecting and deprotecting steps to promote phosphorylation at the 5′ position of the sugar, rather than the 2′ and 3′ hydroxyl groups.

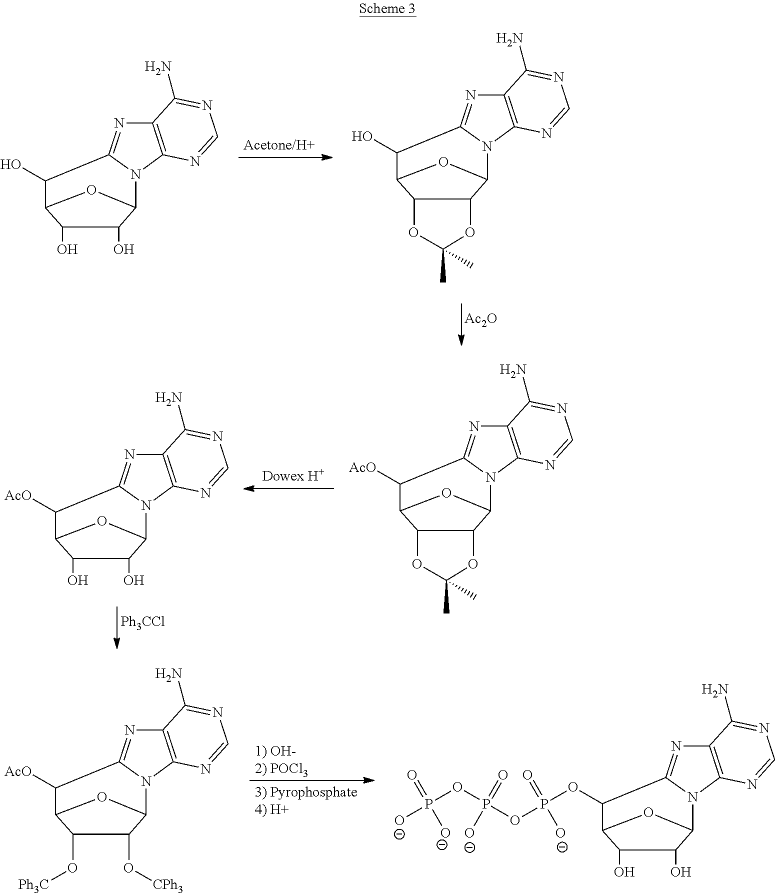

Modified nucleotides can be synthesized in any useful manner. Schemes 4, 5, and 8 provide exemplary methods for synthesizing modified nucleotides having a modified purine nucleobase; and Schemes 6 and 7 provide exemplary methods for synthesizing modified nucleotides having a modified pseudouridine or pseudoisocytidine, respectively.

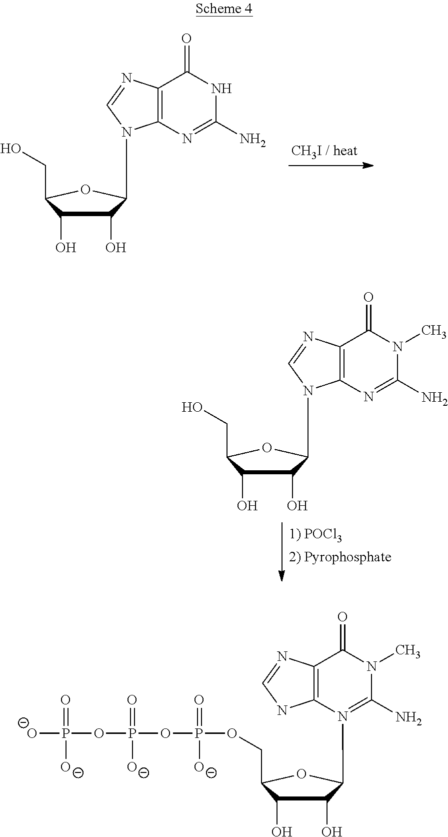

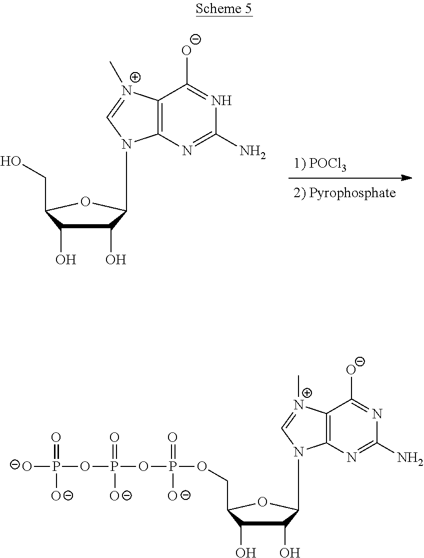

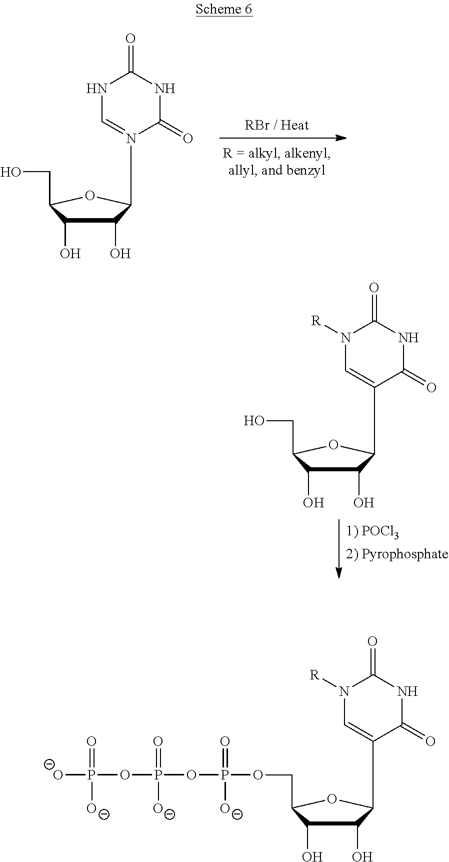

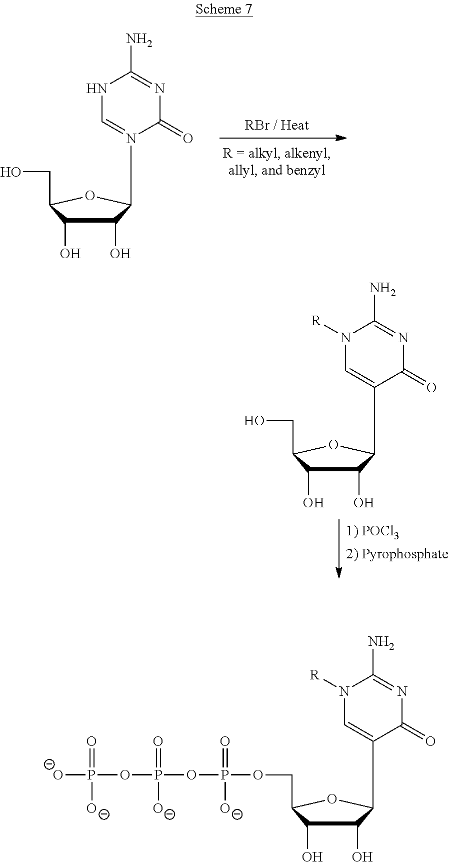

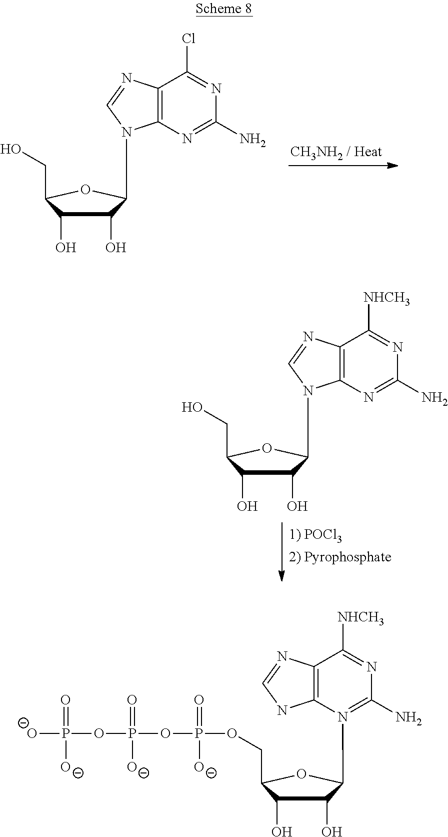

Schemes 9 and 10 provide exemplary syntheses of modified nucleotides. Scheme 11 provides a non-limiting biocatalytic method for producing nucleotides.

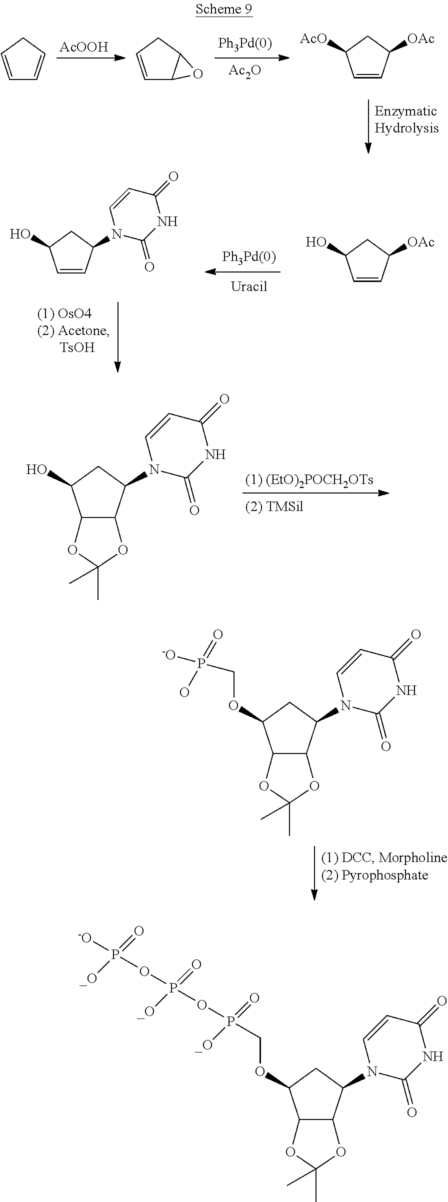

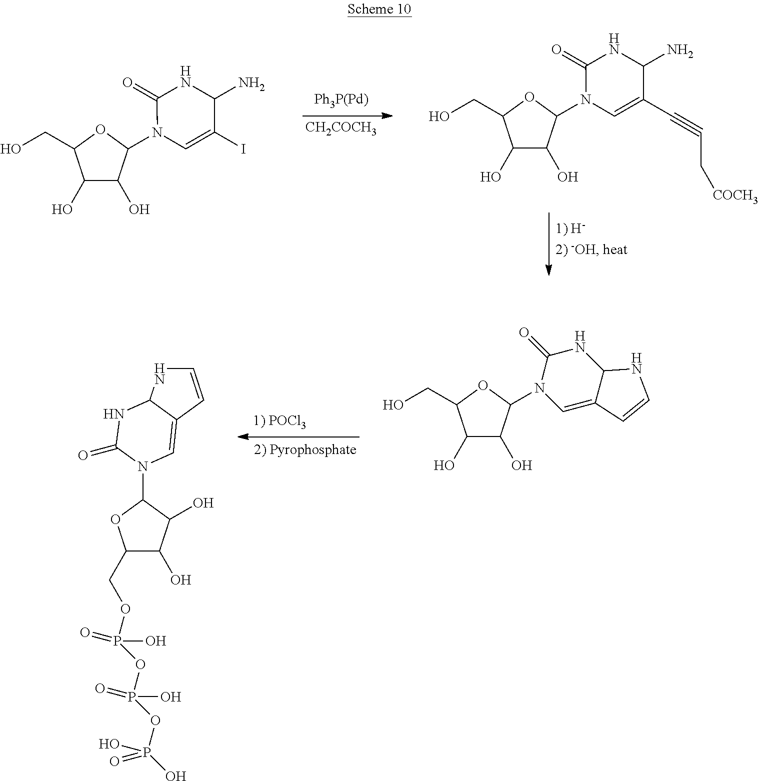

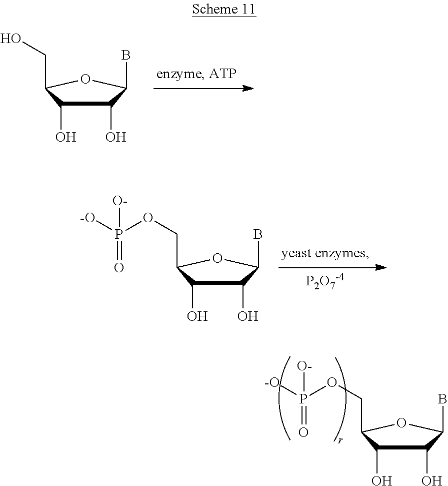

Scheme 12 provides an exemplary synthesis of a modified uracil, where the N1 position is modified with R^{12b}, as provided elsewhere, and the 5′-position of ribose is phosphorylated. T¹, T², R^{12a}, R^{12b}, and r are as provided herein. This synthesis, as well as optimized versions thereof, can be used to modify other pyrimidine nucleobases and purine nucleobases (see e.g., Formulas (b1)-(b43)) and/or to install one or more phosphate groups (e.g., at the 5′ position of the sugar). This alkylating reaction can also be used to include one or more optionally substituted alkyl group at any reactive group (e.g., amino group) in any nucleobase described herein (e.g., the amino groups in the Watson-Crick base-pairing face for cytosine, uracil, adenine, and guanine).

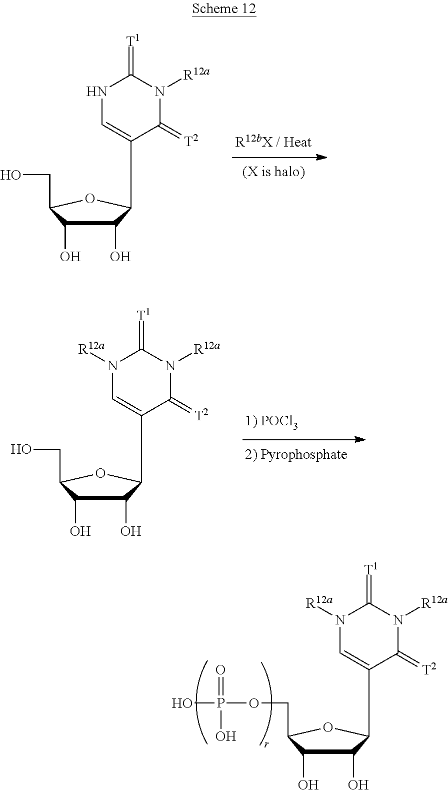

Modified nucleosides and nucleotides can also be prepared according to the synthetic methods described in Ogata et al. Journal of Organic Chemistry 74:2585-2588, 2009; Purmal et al. Nucleic Acids Research 22(1): 72-78, 1994; Fukuhara et al. Biochemistry 1(4): 563-568, 1962; and Xu et al. Tetrahedron 48(9): 1729-1740, 1992, each of which are incorporated by reference in their entirety.

### Modified Nucleic Acids

The present disclosure provides nucleic acids, including RNAs such as mRNAs that contain one or more modified nucleosides (termed “modified nucleic acids”) or nucleotides as described herein, which have useful properties including the significant decrease or lack of a substantial induction of the innate immune response of a cell into which the mRNA is introduced, or the suppression thereof. Because these modified nucleic acids enhance the efficiency of protein production, intracellular retention of nucleic acids, and viability of contacted cells, as well as possess reduced immunogenicity, of these nucleic acids compared to unmodified nucleic acids, having these properties are termed “enhanced nucleic acids” herein.

In addition, the present disclosure provides nucleic acids, which have decreased binding affinity to a major groove interacting, e.g. binding, partner.

The term “nucleic acid,” in its broadest sense, includes any compound and/or substance that is or can be incorporated into an oligonucleotide chain. Exemplary nucleic acids for use in accordance with the present disclosure include, but are not limited to, one or more of DNA, RNA including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc., described in detail herein.

Provided are modified nucleic acids containing a translatable region and one, two, or more than two different nucleoside modifications. In some embodiments, the modified nucleic acid exhibits reduced degradation in a cell into which the nucleic acid is introduced, relative to a corresponding unmodified nucleic acid. Exemplary nucleic acids include ribonucleic acids (RNAs), deoxyribonucleic acids (DNAs), threose nucleic acids (TNAs), glycol nucleic acids (GNAs), locked nucleic acids (LNAs) or a hybrid thereof. In preferred embodiments, the modified nucleic acid includes messenger RNAs (mRNAs). As described herein, the nucleic acids of the present disclosure do not substantially induce an innate immune response of a cell into which the mRNA is introduced.

In certain embodiments, it is desirable to intracellularly degrade a modified nucleic acid introduced into the cell, for example if precise timing of protein production is desired. Thus, the present disclosure provides a modified nucleic acid containing a degradation domain, which is capable of being acted on in a directed manner within a cell.

Other components of nucleic acid are optional, and are beneficial in some embodiments. For example, a 5′ untranslated region (UTR) and/or a 3′UTR are provided, wherein either or both may independently contain one or more different nucleoside modifications. In such embodiments, nucleoside modifications may also be present in the translatable region. Also provided are nucleic acids containing a Kozak sequence.

Additionally, provided are nucleic acids containing one or more intronic nucleotide sequences capable of being excised from the nucleic acid.

### 5′ UTR and Translation Initiation

Natural 5′UTRs bear features which play roles in for translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

By engineering the features typically found in abundantly expressed genes of specific target organs, one can enhance the stability and protein production of the nucleic acids or mRNA of the invention. For example, introduction of 5′ UTR of liver-expressed mRNA, such as albumin, serum amyloid A, Apolipoprotein AB/E, transferrin, alpha fetoprotein, erythropoietin, or Factor VIII, could be used to enhance expression of a nucleic acid molecule, such as a mmRNA, in hepatic cell lines or liver. Likewise, use of 5′ UTR from other tissue-specific mRNA to improve expression in that tissue is possible—for muscle (MyoD, Myosin, Myoglobin, Myogenin, Herculin), for endothelial cells (Tie-1, CD36), for myeloid cells (C/EBP, AML1, G-CSF, GM-CSF, CD11b, MSR, Fr-1, i-NOS), for leukocytes (CD45, CD18), for adipose tissue (CD36, GLUT4, ACRP30, adiponectin) and for lung epithelial cells (SP-A/B/C/D).

Other non-UTR sequences may be incorporated into the 5′ (or 3′ UTR) UTRs. For example, introns or portions of introns sequences may be incorporated into the flanking regions of the nucleic acids or mRNA of the invention. Incorporation of intronic sequences may increase protein production as well as mRNA levels.

### 3′ UTR and the AU Rich Elements

3′UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids or mRNA of the invention. When engineering specific nucleic acids or mRNA, one or more copies of an ARE can be introduced to make nucleic acids or mRNA of the invention less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids or mRNA of the invention and protein production can be assayed at various time points post-transfection. For example, cells can be transfected with different ARE-engineering molecules and by using an ELISA kit to the relevant protein and assaying protein produced at 6 hr, 12 hr, 24 hr, 48 hr, and 7 days post-transfection.

### 3′ UTR and Viral Sequences

Additional viral sequences such as, but not limited to, the translation enhancer sequence of the barley yellow dwarf virus (BYDV-PAV) can be engineered and inserted in the 3′ UTR of the nucleic acids or mRNA of the invention and can stimulate the translation of the construct in vitro and in vivo. Transfection experiments can be conducted in relevant cell lines at and protein production can be assayed by ELISA at 12 hr, 24 hr, 48 hr, 72 hr and day 7 post-transfection.

### 5′ Capping

The 5′ cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA. This 5′-guanylate cap may then be methylated to generate an N7-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

Modifications to the nucleic acids of the present invention may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, Mass.) may be used with α-thio-guanosine nucleotides according to the manufacturer's instructions to create a phosphorothioate linkage in the 5′-ppp-5′ cap. Additional modified guanosine nucleotides may be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/or linked to a nucleic acid molecule.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which may equivalently be designated 3′ O-Me-m7G(5′)ppp(5′)G). The 3′-O atom of the other, unmodified, guanine becomes linked to the 5′-terminal nucleotide of the capped nucleic acid molecule (e.g. an mRNA or mmRNA). The N7- and 3′-O-methylated guanine provides the terminal moiety of the capped nucleic acid molecule (e.g. mRNA or mmRNA).

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

While cap analogs allow for the concomitant capping of a nucleic acid molecule in an in vitro transcription reaction, up to 20% of transcripts remain uncapped. This, as well as the structural differences of a cap analog from an endogenous 5′-cap structures of nucleic acids produced by the endogenous, cellular transcription machinery, may lead to reduced translational competency and reduced cellular stability.

Modified nucleic acids of the invention may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those which, among other things, have enhanced binding of cap binding proteins, increased half life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)N1mpNp (cap 1), 7mG(5′)-ppp(5′)N1mpN2mp (cap 2) and m(7)Gpppm(3)(6,6,2′)Apm(2′)Apm(2′)Cpm(2)(3,2′)Up (cap 4).

Because the modified nucleic acids may be capped post-transcriptionally, and because this process is more efficient, nearly 100% of the modified nucleic acids may be capped. This is in contrast to ˜80% when a cap analog is linked to an mRNA in the course of an in vitro transcription reaction.

According to the present invention, 5′ terminal caps may include endogenous caps or cap analogs. According to the present invention, a 5′ terminal cap may comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### Poly-A Tails

During RNA processing, a long chain of adenine nucleotides (poly-A tail) may be added to a polynucleotide such as an mRNA molecules in order to increase stability. Immediately after transcription, the 3′ end of the transcript may be cleaved to free a 3′ hydroxyl. Then poly-A polymerase adds a chain of adenine nucleotides to the RNA. The process, called polyadenylation, adds a poly-A tail that can be between 100 and 250 residues long.

It has been discovered that unique poly-A tail lengths provide certain advantages to the modified mRNA of the present invention.

Generally, the length of a poly-A tail of the present invention is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the modified mRNA includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In one embodiment, the poly-A tail is designed relative to the length of the overall modified mRNA. This design may be based on the length of the coding region, the length of a particular feature or region (such as a flanking regions), or based on the length of the ultimate product expressed from the modified mRNA.

In this context the poly-A tail may be 10, 20, 30, 40, 50, 60, 70, 80, 90, or 100% greater in length than the modified mRNA or feature thereof. The poly-A tail may also be designed as a fraction of modified mRNA to which it belongs. In this context, the poly-A tail may be 10, 20, 30, 40, 50, 60, 70, 80, or 90% or more of the total length of the molecule or the total length of the molecule minus the poly-A tail. Further, engineered binding sites and conjugation of modified mRNA for Poly-A binding protein may enhance expression.

Additionally, multiple distinct modified mRNA may be linked together to the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In one embodiment, the modified mRNA of the present invention are designed to include a polyA-G Quartet. The G-quartet is a cyclic hydrogen bonded array of four guanine nucleotides that can be formed by G-rich sequences in both DNA and RNA. In this embodiment, the G-quartet is incorporated at the end of the poly-A tail. The resultant modified mRNA molecule is assayed for stability, protein production and other parameters including half-life at various time points. It has been discovered that the polyA-G quartet results in protein production equivalent to at least 75% of that seen using a poly-A tail of 120 nucleotides alone.

### IRES Sequences

Further, provided are nucleic acids containing an internal ribosome entry site (IRES). An IRES may act as the sole ribosome binding site, or may serve as one of multiple ribosome binding sites of an mRNA. An mRNA containing more than one functional ribosome binding site may encode several peptides or polypeptides that are translated independently by the ribosomes (“multicistronic mRNA”). When nucleic acids are provided with an IRES, further optionally provided is a second translatable region. Examples of IRES sequences that can be used according to the present disclosure include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

### Protein Cleavage Signals and Sites

In one embodiment, the nucleic acids of the present invention may include at least one protein cleavage signal containing at least one protein cleavage site. The protein cleavage site may be located at the N-terminus, the C-terminus, at any space between the N- and the C-termini such as, but not limited to, half-way between the N- and C-termini, between the N-terminus and the half way point, between the half way point and the C-terminus, and combinations thereof.

The nucleic acids of the present invention may include, but is not limited to, a proprotein convertase (or prohormone convertase), thrombin or Factor Xa protein cleavage signal. Proprotein convertases are a family of nine proteinases, comprising seven basic amino acid-specific subtilisin-like serine proteinases related to yeast kexin, known as prohormone convertase 1/3 (PC1/3), PC2, furin, PC4, PC5/6, paired basic amino-acid cleaving enzyme 4 (PACE4) and PC7, and two other subtilases that cleave at non-basic residues, called subtilisin kexin isozyme 1 (SKI-1) and proprotein convertase subtilisin kexin 9 (PCSK9). Non-limiting examples of protein cleavage signal amino acid sequences are listing in Table 5. In Table 5, “X” refers to any amino acid, “n” may be 0, 2, 4 or 6 amino acids and “*” refers to the protein cleavage site. In Table 5, SEQ ID NO: 171 refers to when n=4 and SEQ ID NO:172 refers to when n=6.

TABLE 5
Protein Cleavage Site Sequences

| Protein | | |
|---|---|---|
| Cleavage | Amino Acid | SEQ |
| Signal | Cleavage Sequence | ID NO |
| Proprotein | R-X-X-R* | |
| convertase | R-X-K/R-R* | |
| | K/R-Xn-K/R* | 171 and 172 |
| Thrombin | L-V-P-R*-G-S | 173 |
| | L-V-P-R* | |
| | A/F/G/I/L/T/V/M- | |
| | A/F/G/I/L/T/V/W/A-P-R* | |
| Factor Xa | I-E-G-R* | |
| | I-D-G-R* | |
| | A-E-G-R* | |
| | A/F/G/I/L/T/V/M-D/E-G-R* | |

In one embodiment, the nucleic acid and mRNA of the present invention may be engineered such that the nucleic acid or mRNA contain at least one encoded protein cleavage signal. The encoded protein cleavage signal may be located before the start codon, after the start codon, before the coding region, within the coding region such as, but not limited to, half way in the coding region, between the start codon and the half way point, between the half way point and the stop codon, after the coding region, before the stop codon, between two stop codons, after the stop codon and combinations thereof.

In one embodiment, the nucleic acid or mRNA of the present invention may include at least one encoded protein cleavage signal containing at least one protein cleavage site. The encoded protein cleavage signal may include, but is not limited to, a proprotein convertase (or prohormone convertase), thrombin and/or Factor Xa protein cleavage signal. One of skill in the art may use any known methods to determine the appropriate encoded protein cleavage signal to include in the nucleic acid or mRNA of the present invention. For example, starting with the signal of Table 5 and considering the codons known in the art one can design a signal for the nucleic acid which can produce a protein signal in the resulting polypeptide.

In one embodiment, the polypeptides of the present invention include at least one protein cleavage signal and/or site.

As a non-limiting example, U.S. Pat. No. 7,374,930 and U.S. Pub. No. 20090227660, herein incorporated by reference in their entireties, use a furin cleavage site to cleave the N-terminal methionine of GLP-1 in the expression product from the Golgi apparatus of the cells. In one embodiment, the polypeptides of the present invention include at least one protein cleavage signal and/or site with the proviso that the polypeptide is not GLP-1.

In one embodiment, the nucleic acid or mRNA of the present invention includes at least one encoded protein cleavage signal and/or site.

In one embodiment, the nucleic acid or mRNA of the present invention includes at least one encoded protein cleavage signal and/or site with the proviso that the nucleic acid or mRNA does not encode GLP-1.

In one embodiment, the nucleic acid or mRNA of the present invention may include more than one coding region. Where multiple coding regions are present in the nucleic acid or mRNA of the present invention, the multiple coding regions may be separated by encoded protein cleavage sites. As a non-limiting example, the nucleic acid or mRNA may be signed in an ordered pattern. On such pattern follows AXBY form where A and B are coding regions which may be the same or different coding regions and/or may encode the same or different polypeptides, and X and Y are encoded protein cleavage signals which may encode the same or different protein cleavage signals. A second such pattern follows the form AXYBZ where A and B are coding regions which may be the same or different coding regions and/or may encode the same or different polypeptides, and X, Y and Z are encoded protein cleavage signals which may encode the same or different protein cleavage signals. A third pattern follows the form ABXCY where A, B and C are coding regions which may be the same or different coding regions and/or may encode the same or different polypeptides, and X and Y are encoded protein cleavage signals which may encode the same or different protein cleavage signals.

In one embodiment, the nucleic acid or mRNA can also contain sequences that encode protein cleavage sites so that the nucleic acid or mRNA can be released from a carrier.

### Cyclic Modified RNA

According to the present invention, a nucleic acid or modified RNA may be cyclized, or concatemerized, to generate a translation competent molecule to assist interactions between poly-A binding proteins and 5′-end binding proteins. The mechanism of cyclization or concatemerization may occur through at least 3 different routes: 1) chemical, 2) enzymatic, and 3) ribozyme catalyzed. The newly formed 5′-/3′-linkage may be intramolecular or intermolecular.

In the first route, the 5′-end and the 3′-end of the nucleic acid contain chemically reactive groups that, when close together, form a new covalent linkage between the 5′-end and the 3′-end of the molecule. The 5′-end may contain an NETS-ester reactive group and the 3′-end may contain a 3′-amino-terminated nucleotide such that in an organic solvent the 3′-amino-terminated nucleotide on the 3′-end of a synthetic mRNA molecule will undergo a nucleophilic attack on the 5′-NHS-ester moiety forming a new 5′-/3′-amide bond.

In the second route, T4 RNA ligase may be used to enzymatically link a 5′-phosphorylated nucleic acid molecule to the 3′-hydroxyl group of a nucleic acid forming a new phosphorodiester linkage. In an example reaction, 1 μg of a nucleic acid molecule is incubated at 37° C. for 1 hour with 1-10 units of T4 RNA ligase (New England Biolabs, Ipswich, Mass.) according to the manufacturer's protocol. The ligation reaction may occur in the presence of a split oligonucleotide capable of base-pairing with both the 5′- and 3′-region in juxtaposition to assist the enzymatic ligation reaction.

In the third route, either the 5′- or 3′-end of the cDNA template encodes a ligase ribozyme sequence such that during in vitro transcription, the resultant nucleic acid molecule can contain an active ribozyme sequence capable of ligating the 5′-end of a nucleic acid molecule to the 3′-end of a nucleic acid molecule. The ligase ribozyme may be derived from the Group I Intron, Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

### Modified RNA Multimers

According to the present invention, multiple distinct nucleic acids or modified RNA may be linked together through the 3′-end using nucleotides which are modified at the 3′-terminus. Chemical conjugation may be used to control the stoichiometry of delivery into cells. For example, the glyoxylate cycle enzymes, isocitrate lyase and malate synthase, may be supplied into HepG2 cells at a 1:1 ratio to alter cellular fatty acid metabolism. This ratio may be controlled by chemically linking nucleic acids or modified RNA using a 3′-azido terminated nucleotide on one nucleic acids or modified RNA species and a C5-ethynyl or alkynyl-containing nucleotide on the opposite nucleic acids or modified RNA species. The modified nucleotide is added post-transcriptionally using terminal transferase (New England Biolabs, Ipswich, Mass.) according to the manufacturer's protocol. After the addition of the 3′-modified nucleotide, the two nucleic acids or modified RNA species may be combined in an aqueous solution, in the presence or absence of copper, to form a new covalent linkage via a click chemistry mechanism as described in the literature.

In another example, more than two polynucleotides may be linked together using a functionalized linker molecule. For example, a functionalized saccharide molecule may be chemically modified to contain multiple chemical reactive groups (SH—, NH₂—, N₃, etc. . . . ) to react with the cognate moiety on a 3′-functionalized mRNA molecule (i.e., a 3′-maleimide ester, 3′-NHS-ester, alkynyl). The number of reactive groups on the modified saccharide can be controlled in a stoichiometric fashion to directly control the stoichiometric ratio of conjugated nucleic acid or mRNA.

### Modified RNA Conjugates and Combinations

In order to further enhance protein production, nucleic acids or modified RNA of the present invention can be designed to be conjugated to other polynucleotides, dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases, proteins, e.g., glycoproteins, or peptides, e.g., molecules having a specific affinity for a co-ligand, or antibodies e.g., an antibody, that binds to a specified cell type such as a cancer cell, endothelial cell, or bone cell, hormones and hormone receptors, non-peptidic species, such as lipids, lectins, carbohydrates, vitamins, cofactors, or a drug.

Conjugation may result in increased stability and/or half life and may be particularly useful in targeting the nucleic acids or modified RNA to specific sites in the cell, tissue or organism.

According to the present invention, the nucleic acids or modified RNA may be administered with, or further encode one or more of RNAi agents, siRNAs, shRNAs, miRNAs, miRNA binding sites, antisense RNAs, ribozymes, catalytic DNA, tRNA, RNAs that induce triple helix formation, aptamers or vectors, and the like.

Bifunctional mmRNA

In one embodiment of the invention are bifunctional polynucleotides (e.g., bifunctional nucleic acids or bifunctional modified RNA). As the name implies, bifunctional polynucleotides are those having or capable of at least two functions. These molecules may also by convention be referred to as multi-functional.

The multiple functionalities of bifunctional polynucleotides may be encoded by the RNA (the function may not manifest until the encoded product is translated) or may be a property of the polynucleotide itself. It may be structural or chemical. Bifunctional modified polynucleotides may comprise a function that is covalently or electrostatically associated with the polynucleotides. Further, the two functions may be provided in the context of a complex of a modified RNA and another molecule.

Bifunctional polynucleotides may encode peptides which are anti-proliferative. These peptides may be linear, cyclic, constrained or random coil. They may function as aptamers, signaling molecules, ligands or mimics or mimetics thereof. Anti-proliferative peptides may, as translated, be from 3 to 50 amino acids in length. They may be 5-40, 10-30, or approximately 15 amino acids long. They may be single chain, multichain or branched and may form complexes, aggregates or any multi-unit structure once translated.

### Noncoding Nucleic Acids and Modified RNA

As described herein, provided are nucleic acids or modified RNA having sequences that are partially or substantially not translatable, e.g., having a noncoding region. Such molecules are generally not translated, but can exert an effect on protein production by one or more of binding to and sequestering one or more translational machinery components such as a ribosomal protein or a transfer RNA (tRNA), thereby effectively reducing protein expression in the cell or modulating one or more pathways or cascades in a cell which in turn alters protein levels. The nucleic acids or mRNA may contain or encode one or more long noncoding RNA (lncRNA, or lincRNA) or portion thereof, a small nucleolar RNA (sno-RNA), micro RNA (miRNA), small interfering RNA (siRNA) or Piwi-interacting RNA (piRNA).

### Terminal Architecture Modifications: 5′-Capping

The 5′ cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous eukaryotic cellular messenger RNA (mRNA) molecules contain a 5′-cap structure on the 5′-end of a mature mRNA molecule. The 5′-cap may contain a 5′-5′-triphosphate linkage (a 5′-ppp-5′-triphosphate linkage) between the 5′-most nucleotide and a terminal guanine nucleotide. The conjugated guanine nucleotide is methylated at the N7 position. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

Modifications to the nucleic acids or mRNA of the present invention may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, Mass.) may be used with α-thio-guanosine nucleotides according to the manufacturer's instructions to create a phosphorothioate linkage in the 5′-ppp-5′ cap. Additional modified guanosine nucleotides may be used such as α-methyl-phosphonate and seleno-phosphate nucleotides.

Additional modifications include methylation of the ultimate and penultimate most 5′-nucleotides on the 2′-hydroxyl group. The 5′-cap structure is responsible for binding the mRNA Cap Binding Protein (CBP), which is responsibility for mRNA stability in the cell and translation competency. Multiple distinct 5′-cap structures can be used to generate the 5′-cap of a synthetic mRNA molecule.

Many chemical cap analogs are used to co-transcriptionally cap a synthetic mRNA molecule. Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/linked to a nucleic acid molecule.

For example, the Anti-Reverse Cap Analog (ARCA) cap contains a 5′-5′-triphosphate guanine-guanine linkage where one guanine contains an N7 methyl group as well as a 3′-O-methyl group (i.e., N7,3′-O-dimethyl-guanosine-5′-triphosphate-5′-guanosine (m⁷G-3′mppp-G; which may equivalently be designated 3′ O-Me-m7G(5)ppp(5′)G)). The 3′-O atom of the other, unmodified, guanine becomes linked to the 5′-terminal nucleotide of the capped nucleic acid molecule (e.g. an mRNA or mmRNA). The N7- and 3′-O-methylated guanine provides the terminal moiety of the capped nucleic acid molecule (e.g. mRNA or mmRNA).

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

While chemical cap analogs allow for the concomitant capping of an RNA molecule, up 20% of transcripts remain uncapped and the synthetic cap analog is not identical to an endogenous 5′-cap structure of an authentic cellular mRNA. This may lead to reduced translationally-competency and reduced cellular stability.

Synthetic mRNA molecules may also be capped post-transcriptionally using enzymes responsible for generating a more authentic 5′-cap structure. As used herein the phrase “more authentic” refers to a feature that closely mirrors or mimics, either structurally or functionally an endogenous or wild type feature. Non-limiting examples of more authentic 5′ cap structures of the present invention are those which, among other things, have enhanced binding of cap binding proteins, increased half life, reduced susceptibility to 5′ endonucleases and/or reduced 5′ decapping. For example, recombinant Vaccinia Virus Capping Enzyme and recombinant 2′-O-methyltransferase enzyme can create a canonical 5′-5′-triphosphate linkage between the 5′-most nucleotide of an mRNA and a guanine nucleotide where the guanine contains an N7 methylation and the ultimate 5′-nucleotide contains a 2′-O-methyl. Such a structure is termed the Cap1 structure. This results in a cap with higher translational-competency and cellular stability and reduced activation of cellular pro-inflammatory cytokines, as compared, e.g., to other 5′cap analog structures known in the art. Cap structures include 7mG(5′)ppp(5′)N,pN2p (cap 0), 7mG(5′)ppp(5′)N1mpNp (cap 1), and 7mG(5′)-ppp(5′)N1mpN2mp (cap 2).

Because the synthetic mRNA is capped post-transcriptionally, and because this process is more efficient, nearly 100% of the mRNA molecules may be capped. This is in contrast to ˜80% when a cap analog is linked to synthetic mRNAs in the course of an in vitro transcript reaction.

According to the present invention, 5′ terminal caps may include endogenous caps or cap analogs. According to the present invention, a 5′ terminal cap may comprise a guanine analog. Useful guanine analogs include inosine, N1-methyl-guanosine, 2′fluoro-guanosine, 7-deaza-guanosine, 8-oxo-guanosine, 2-amino-guanosine, LNA-guanosine, and 2-azido-guanosine.

### Terminal Architecture Modifications: Poly-A Tails

During RNA processing, a long chain of adenine nucleotides (poly-A tail) is normally added to a messenger RNA (mRNA) molecules to increase the stability of the molecule. Immediately after transcription, the 3′ end of the transcript is cleaved to free a 3′ hydroxyl. Then poly-A polymerase adds a chain of adenine nucleotides to the RNA. The process, called polyadenylation, adds a poly-A tail that is between 100 and 250 residues long.

It has been discovered that unique poly-A tail lengths provide certain advantages to the modified RNAs of the present invention.

Generally, the length of a poly-A tail of the present invention is greater than 30 nucleotides in length. In another embodiment, the poly-A tail is greater than 35 nucleotides in length. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 60 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1700 nucleotides. In another embodiment, the length is at least 1800 nucleotides. In another embodiment, the length is at least 1900 nucleotides. In another embodiment, the length is at least 2000 nucleotides. In another embodiment, the length is at least 2500 nucleotides. In another embodiment, the length is at least 3000 nucleotides.

In some embodiments, the nucleic acid or mRNA includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In one embodiment, the poly-A tail is designed relative to the length of the overall modified RNA molecule. This design may be based on the length of the coding region of the modified RNA, the length of a particular feature or region of the modified RNA (such as the mRNA), or based on the length of the ultimate product expressed from the modified RNA. When relative to any additional feature of the modified RNA (e.g., other than the mRNA portion which includes the poly-A tail) the poly-A tail may be 10, 20, 30, 40, 50, 60, 70, 80, 90 or 100% greater in length than the additional feature. The poly-A tail may also be designed as a fraction of the modified RNA to which it belongs. In this context, the poly-A tail may be 10, 20, 30, 40, 50, 60, 70, 80, or 90% or more of the total length of the construct or the total length of the construct minus the poly-A tail. Further, engineered binding sites and conjugation of nucleic acids or mRNA for Poly-A binding protein may enhance expression.

Additionally, multiple distinct nucleic acids or mRNA may be linked together to the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In one embodiment, the nucleic acids or mRNA of the present invention are designed to include a polyA-G Quartet. The G-quartet is a cyclic hydrogen bonded array of four guanine nucleotides that can be formed by G-rich sequences in both DNA and RNA. In this embodiment, the G-quartet is incorporated at the end of the poly-A tail. The resultant nucleic acid or mRNA may be assayed for stability, protein production and other parameters including half-life at various time points. It has been discovered that the polyA-G quartet results in protein production equivalent to at least 75% of that seen using a poly-A tail of 120 nucleotides alone.

### Modified Nucleotides, Nucleosides and Polynucleotides of the Invention

Herein, in a nucleotide, nucleoside polynucleotide (such as the nucleic acids of the invention, e.g., modified RNA, modified nucleic acid molecule, modified RNAs, nucleic acid and modified nucleic acids), the terms “modification” or, as appropriate, “modified” refer to modification with respect to A, G, U or C ribonucleotides. Generally, herein, these terms are not intended to refer to the ribonucleotide modifications in naturally occurring 5′-terminal mRNA cap moieties. In a polypeptide, the term “modification” refers to a modification as compared to the canonical set of 20 amino acids, moiety.

The modifications may be various distinct modifications. In some embodiments, where the nucleic acids or modified RNA, the coding region, the flanking regions and/or the terminal regions may contain one, two, or more (optionally different) nucleoside or nucleotide modifications. In some embodiments, a modified nucleic acids or modified RNA introduced to a cell may exhibit reduced degradation in the cell, as compared to an unmodified nucleic acids or modified RNA.

The nucleic acids or modified RNA can include any useful modification, such as to the sugar, the nucleobase, or the internucleoside linkage (e.g. to a linking phosphate/to a phosphodiester linkage/to the phosphodiester backbone). In certain embodiments, modifications (e.g., one or more modifications) are present in each of the sugar and the internucleoside linkage. Modifications according to the present invention may be modifications of ribonucleic acids (RNAs) to deoxyribonucleic acids (DNAs), e.g., the substitution of the 2′OH of the ribofuranysyl ring to 2′H, threose nucleic acids (TNAs), glycol nucleic acids (GNAs), peptide nucleic acids (PNAs), locked nucleic acids (LNAs) or hybrids thereof). Additional modifications are described herein.

As described herein, the nucleic acids or modified RNA of the invention do not substantially induce an innate immune response of a cell into which the nucleic acids or modified RNA (e.g., mRNA) is introduced. Features of an induced innate immune response include 1) increased expression of pro-inflammatory cytokines, 2) activation of intracellular PRRs (RIG-I, MDA5, etc, and/or 3) termination or reduction in protein translation.

In certain embodiments, it may desirable for a modified nucleic acid molecule introduced into the cell to be degraded intracellulary. For example, degradation of a modified nucleic acid molecule may be preferable if precise timing of protein production is desired. Thus, in some embodiments, the invention provides a modified nucleic acid molecule containing a degradation domain, which is capable of being acted on in a directed manner within a cell.

In another aspect, the present disclosure provides nucleic acids or modified RNA comprising a nucleoside or nucleotide that can disrupt the binding of a major groove interacting, e.g. binding, partner with the nucleic acids or modified RNA (e.g., where the modified nucleotide has decreased binding affinity to major groove interacting partner, as compared to an unmodified nucleotide).

The nucleic acids or modified RNA can optionally include other agents (e.g., RNAi-inducing agents, RNAi agents, siRNAs, shRNAs, miRNAs, antisense RNAs, ribozymes, catalytic DNA, tRNA, RNAs that induce triple helix formation, aptamers, vectors, etc.). In some embodiments, the nucleic acids or modified RNA may include one or more messenger RNAs (mRNAs) having one or more modified nucleoside or nucleotides (i.e., modified mRNA molecules). Details for these nucleic acids or modified RNA follow.

### Nucleic Acids or Modified RNA

The nucleic acids or modified RNA of the invention includes a first region of linked nucleosides encoding a polypeptide of interest, a first flanking region located at the 5′ terminus of the first region, and a second flanking region located at the 3′ terminus of the first region. The first region of linked nucleosides may be a translatable region.

In some embodiments, the nucleic acids or modified RNA (e.g., the first region, first flanking region, or second flanking region) includes n number of linked nucleosides having Formula (Ia) or Formula (Ia-1):

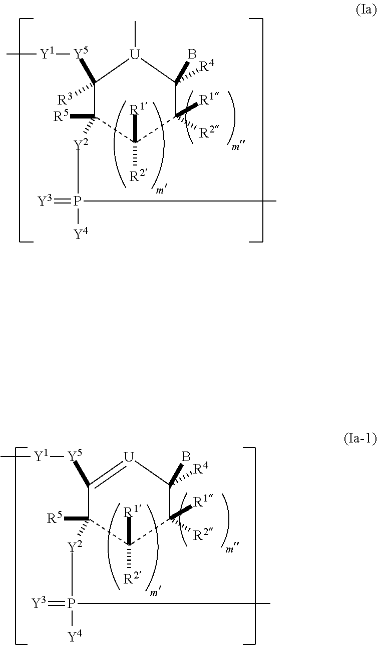

or a pharmaceutically acceptable salt or stereoisomer thereof, wherein U is O, S, N(R^{U})ₙᵤ, or C(R^{U})ₙᵤ, wherein nu is an integer from 0 to 2 and each R^{U} is, independently, H, halo, or optionally substituted alkyl;

- - - is a single bond or absent;

each of R^{1′}, R^{2′}, R^{1″}, R^{2″}, R¹, R², R³, R⁴, and R⁵, if present, is, independently, H, halo, hydroxy, thiol, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted hydroxyalkoxy, optionally substituted amino, azido, optionally substituted aryl, optionally substituted aminoalkyl, optionally substituted aminoalkenyl, optionally substituted aminoalkynyl, or absent; wherein the combination of R³ with one or more of R^{1′}, R^{1″}, R^{2′}, R^{2″}, or R⁵ (e.g., the combination of R^{1′} and R³, the combination of R^{1″} and R³, the combination of R^{2′} and R³, the combination of R^{2″} and R³, or the combination of R⁵ and R³) can join together to form optionally substituted alkylene or optionally substituted heteroalkylene and, taken together with the carbons to which they are attached, provide an optionally substituted heterocyclyl (e.g., a bicyclic, tricyclic, or tetracyclic heterocyclyl); wherein the combination of R⁵ with one or more of R^{1′}, R^{1″}, R^{2′}, or R^{2″} (e.g., the combination of R^{1′} and R⁵, the combination of R^{1″} and R⁵, the combination of R^{2′} and R⁵, or the combination of R^{2″} and R⁵) can join together to form optionally substituted alkylene or optionally substituted heteroalkylene and, taken together with the carbons to which they are attached, provide an optionally substituted heterocyclyl (e.g., a bicyclic, tricyclic, or tetracyclic heterocyclyl); and wherein the combination of R⁴ and one or more of R^{1′}, R^{1″}, R^{2′}, R^{2″}, R³, or R⁵ can join together to form optionally substituted alkylene or optionally substituted heteroalkylene and, taken together with the carbons to which they are attached, provide an optionally substituted heterocyclyl (e.g., a bicyclic, tricyclic, or tetracyclic heterocyclyl);

each of m′ and m″ is, independently, an integer from 0 to 3 (e.g., from 0 to 2, from 0 to 1, from 1 to 3, or from 1 to 2);

each of Y¹, Y², and Y³, is, independently, O, S, Se, —NR^{N1}—, optionally substituted alkylene, or optionally substituted heteroalkylene, wherein R^{N1} is H, optionally substituted alkyl, optionally substituted alkenyl, optionally substituted alkynyl, optionally substituted aryl, or absent;

each Y⁴ is, independently, H, hydroxy, thiol, boranyl, optionally substituted alkyl, optionally substituted alkenyl, optionally substituted alkynyl, optionally substituted alkoxy, optionally substituted alkenyloxy, optionally substituted alkynyloxy, optionally substituted thioalkoxy, optionally substituted alkoxyalkoxy, or optionally substituted amino;

each Y⁵ is, independently, O, S, Se, optionally substituted alkylene (e.g., methylene), or optionally substituted heteroalkylene;

n is an integer from 1 to 100,000; and

B is a nucleobase (e.g., a purine, a pyrimidine, or derivatives thereof), wherein the combination of B and R^{1′}, the combination of B and R^{2′}, the combination of B and R^{1″}, or the combination of B and R^{2″} can, taken together with the carbons to which they are attached, optionally form a bicyclic group (e.g., a bicyclic heterocyclyl) or wherein the combination of B, R^{1″}, and R³ or the combination of B, R^{2″}, and R³ can optionally form a tricyclic or tetracyclic group (e.g., a tricyclic or tetracyclic heterocyclyl, such as in Formula (IIo)-(IIp) herein).

In some embodiments, the nucleic acids or modified RNA includes a modified ribose. In some embodiments, the nucleic acids or modified RNA (e.g., the first region, the first flanking region, or the second flanking region) includes n number of linked nucleosides having Formula (Ia-2)-(Ia-5) or a pharmaceutically acceptable salt or stereoisomer thereof

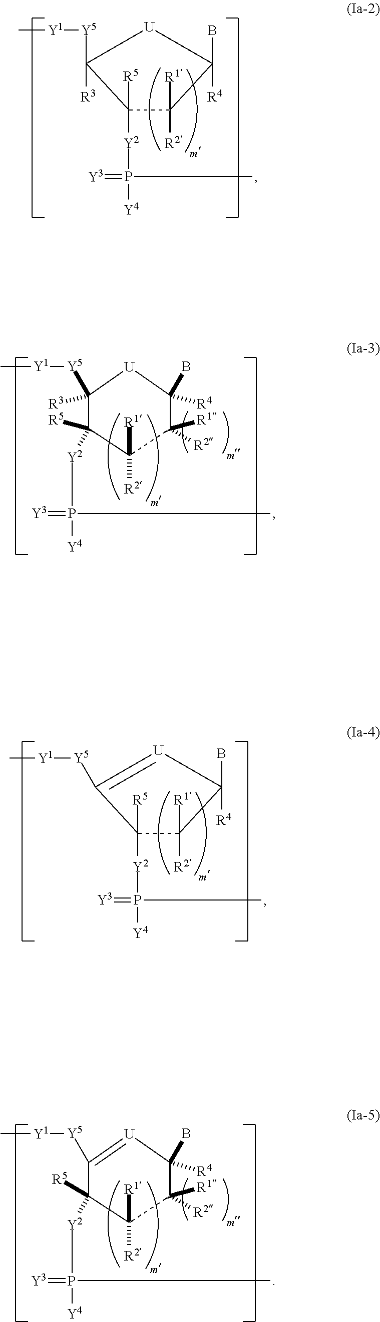

In some embodiments, the nucleic acids or modified RNA (e.g., the first region, the first flanking region, or the second flanking region) includes n number of linked nucleosides having Formula (Ib) or Formula (Ib-1):

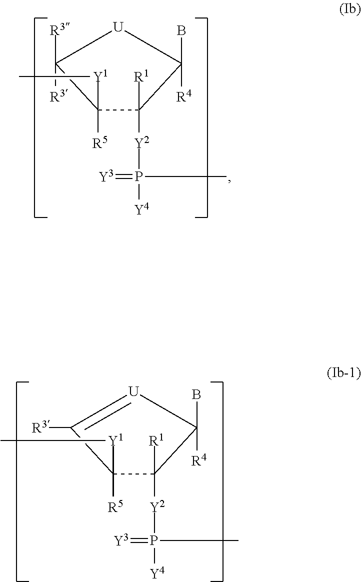

or a pharmaceutically acceptable salt or stereoisomer thereof, wherein

U is O, S, N(R^{U})ₙᵤ, or C(R^{U})ₙᵤ, wherein nu is an integer from 0 to 2 and each R^{U} is, independently, H, halo, or optionally substituted alkyl;

- - - is a single bond or absent;

each of R¹, R^{3′}, R^{3″}, and R⁴ is, independently, H, halo, hydroxy, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted hydroxyalkoxy, optionally substituted amino, azido, optionally substituted aryl, optionally substituted aminoalkyl, optionally substituted aminoalkenyl, optionally substituted aminoalkynyl, or absent; and wherein the combination of R¹ and R^{3′} or the combination of R¹ and R^{3″} can be taken together to form optionally substituted alkylene or optionally substituted heteroalkylene (e.g., to produce a locked nucleic acid);

each R⁵ is, independently, H, halo, hydroxy, optionally substituted alkyl, optionally substituted alkoxy, optionally substituted alkenyloxy, optionally substituted alkynyloxy, optionally substituted aminoalkoxy, optionally substituted alkoxyalkoxy, or absent;

each of Y¹, Y², and Y³ is, independently, O, S, Se, NR^{N1}—, optionally substituted alkylene, or optionally substituted heteroalkylene, wherein R^{N1} is H, optionally substituted alkyl, optionally substituted alkenyl, optionally substituted alkynyl, or optionally substituted aryl;

each Y⁴ is, independently, H, hydroxy, thiol, boranyl, optionally substituted alkyl, optionally substituted alkenyl, optionally substituted alkynyl, optionally substituted alkoxy, optionally substituted alkenyloxy, optionally substituted alkynyloxy, optionally substituted alkoxyalkoxy, or optionally substituted amino;

n is an integer from 1 to 100,000; and

B is a nucleobase.

In some embodiments, the nucleic acids or modified RNA (e.g., the first region, first flanking region, or second flanking region) includes n number of linked nucleosides having Formula (Ic):

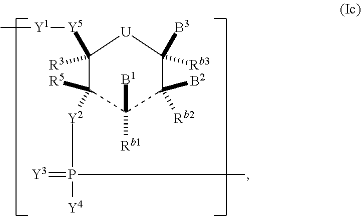

or a pharmaceutically acceptable salt or stereoisomer thereof, wherein

U is O, S, N(R^{U})ₙᵤ, or C(R^{U})ₙᵤ, wherein nu is an integer from 0 to 2 and each R^{U} is, independently, H, halo, or optionally substituted alkyl;

- - - is a single bond or absent;

each of B¹, B², and B³ is, independently, a nucleobase (e.g., a purine, a pyrimidine, or derivatives thereof, as described herein), H, halo, hydroxy, thiol, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted hydroxyalkoxy, optionally substituted amino, azido, optionally substituted aryl, optionally substituted aminoalkyl, optionally substituted aminoalkenyl, or optionally substituted aminoalkynyl, wherein one and only one of B¹, B², and B³ is a nucleobase;

each of R^{b1}, R^{b2}, R^{b3}, R³, and R⁵ is, independently, H, halo, hydroxy, thiol, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted hydroxyalkoxy, optionally substituted amino, azido, optionally substituted aryl, optionally substituted aminoalkyl, optionally substituted aminoalkenyl, or optionally substituted aminoalkynyl;

each of Y¹, Y², and Y³, is, independently, O, S, Se, —NR^{N1}—, optionally substituted alkylene, or optionally substituted heteroalkylene, wherein R^{N1} is H, optionally substituted alkyl, optionally substituted alkenyl, optionally substituted alkynyl, or optionally substituted aryl;

each Y⁴ is, independently, H, hydroxy, thiol, boranyl, optionally substituted alkyl, optionally substituted alkenyl, optionally substituted alkynyl, optionally substituted alkoxy, optionally substituted alkenyloxy, optionally substituted alkynyloxy, optionally substituted thioalkoxy, optionally substituted alkoxyalkoxy, or optionally substituted amino;

each Y⁵ is, independently, O, S, Se, optionally substituted alkylene (e.g., methylene), or optionally substituted heteroalkylene;

n is an integer from 1 to 100,000; and

wherein the ring including U can include one or more double bonds.

In particular embodiments, the ring including U does not have a double bond between U—CB³R^{b3} or between CB³R^{b3}—C^{B2}R^{b2}.

In some embodiments, the nucleic acids or modified RNA (e.g., the first region, first flanking region, or second flanking region) includes n number of linked nucleosides having Formula (Id):

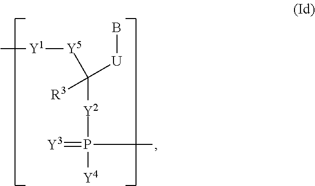

or a pharmaceutically acceptable salt or stereoisomer thereof, wherein U is O, S, N(R^{U})ₙᵤ, or C(R^{U})ₙᵤ, wherein nu is an integer from 0 to 2 and each R^{U} is, independently, H, halo, or optionally substituted alkyl;

each R³ is, independently, H, halo, hydroxy, thiol, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted hydroxyalkoxy, optionally substituted amino, azido, optionally substituted aryl, optionally substituted aminoalkyl, optionally substituted aminoalkenyl, or optionally substituted aminoalkynyl;

each of Y¹, Y², and Y³, is, independently, O, S, Se, —NR^{N1}—, optionally substituted alkylene, or optionally substituted heteroalkylene, wherein R^{N1} is H, optionally substituted alkyl, optionally substituted alkenyl, optionally substituted alkynyl, or optionally substituted aryl;

each Y⁴ is, independently, H, hydroxy, thiol, boranyl, optionally substituted alkyl, optionally substituted alkenyl, optionally substituted alkynyl, optionally substituted alkoxy, optionally substituted alkenyloxy, optionally substituted alkynyloxy, optionally substituted thioalkoxy, optionally substituted alkoxyalkoxy, or optionally substituted amino;

each Y⁵ is, independently, O, S, optionally substituted alkylene (e.g., methylene), or optionally substituted heteroalkylene;

n is an integer from 1 to 100,000; and

B is a nucleobase (e.g., a purine, a pyrimidine, or derivatives thereof).

In some embodiments, the polynucleotide includes n number of linked nucleosides having Formula (Ie):

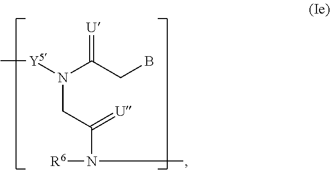

or a pharmaceutically acceptable salt or stereoisomer thereof,

wherein each of U′ and U″ is, independently, O, S, N(R^{U})ₙᵤ, or C(R^{U})ₙᵤ, wherein nu is an integer from 0 to 2 and each RU is, independently, H, halo, or optionally substituted alkyl;

each R⁶ is, independently, H, halo, hydroxy, thiol, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted hydroxyalkoxy, optionally substituted amino, azido, optionally substituted aryl, optionally substituted aminoalkyl, optionally substituted aminoalkenyl, or optionally substituted aminoalkynyl;

each Y^{5′} is, independently, O, S, optionally substituted alkylene (e.g., methylene or ethylene), or optionally substituted heteroalkylene;

n is an integer from 1 to 100,000; and

B is a nucleobase (e.g., a purine, a pyrimidine, or derivatives thereof).

In some embodiments, the nucleic acids or modified RNA (e.g., the first region, first flanking region, or second flanking region) includes n number of linked nucleosides having Formula (If) or (If-1):

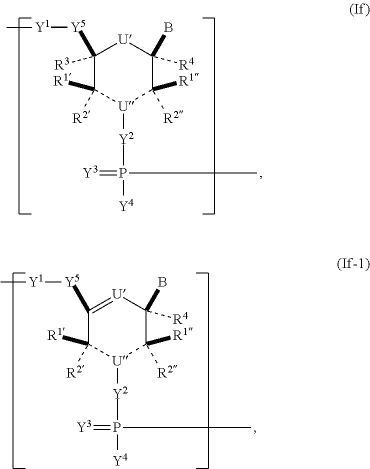

or a pharmaceutically acceptable salt or stereoisomer thereof,

wherein each of U′ and U″ is, independently, O, S, N, N(R^{U})ₙᵤ, or C(R^{U})ₙᵤ, wherein nu is an integer from 0 to 2 and each R^{U} is, independently, H, halo, or optionally substituted alkyl (e.g., U′ is O and U″ is N);

- - - is a single bond or absent;

each of R^{1′}, R^{2′}, R^{1″}, R^{2″}, R³, and R⁴ is, independently, H, halo, hydroxy, thiol, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted hydroxyalkoxy, optionally substituted amino, azido, optionally substituted aryl, optionally substituted aminoalkyl, optionally substituted aminoalkenyl, optionally substituted aminoalkynyl, or absent; and wherein the combination of R^{1′} and R³, the combination of R^{1″} and R³, the combination of R^{2′} and R³, or the combination of R^{2″} and R³ can be taken together to form optionally substituted alkylene or optionally substituted heteroalkylene (e.g., to produce a locked nucleic acid); each of m′ and m″ is, independently, an integer from 0 to 3 (e.g., from 0 to 2, from 0 to 1, from 1 to 3, or from 1 to 2);

each of Y¹, Y², and Y³, is, independently, O, S, Se, —NR^{N1}—, optionally substituted alkylene, or optionally substituted heteroalkylene, wherein R^{N1} is H, optionally substituted alkyl, optionally substituted alkenyl, optionally substituted alkynyl, optionally substituted aryl, or absent;

each Y⁴ is, independently, H, hydroxy, thiol, boranyl, optionally substituted alkyl, optionally substituted alkenyl, optionally substituted alkynyl, optionally substituted alkoxy, optionally substituted alkenyloxy, optionally substituted alkynyloxy, optionally substituted thioalkoxy, optionally substituted alkoxyalkoxy, or optionally substituted amino;

each Y⁵ is, independently, O, S, Se, optionally substituted alkylene (e.g., methylene), or optionally substituted heteroalkylene;

n is an integer from 1 to 100,000; and

B is a nucleobase (e.g., a purine, a pyrimidine, or derivatives thereof).

In some embodiments of the nucleic acids or modified RNA (e.g., Formulas (Ia)-(Ia-5), (Ib)-(If-1), (IIa)-(IIp), (IIb-1), (IIb-2), (IIc-1)-(IIc-2), (IIn-1), (IIn-2), (IVa)-(IV1), and (IXa)-(IXr)), the ring including U has one or two double bonds.

In some embodiments of the nucleic acids or modified RNA (e.g., Formulas (Ia)-Ia-5), (Ib)-(If-1), (IIa)-(IIp), (IIb-1), (IIb-2), (IIc-1)-(IIc-2), (IIn-1), (IIn-2), (IVa)-(IV1), and (IXa)-(IXr)), each of R¹, R^{1′}, and R^{1″}, if present, is H. In further embodiments, each of R², R^{2′}, and R²″, if present, is, independently, H, halo (e.g., fluoro), hydroxy, optionally substituted alkoxy (e.g., methoxy or ethoxy), or optionally substituted alkoxyalkoxy. In particular embodiments, alkoxyalkoxy is —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl). In some embodiments, s2 is 0, s1 is 1 or 2, s3 is 0 or 1, and R′ is C₁₋₆ alkyl.

In some embodiments of the nucleic acids or modified RNA (e.g., Formulas (Ia)-(Ia-5), (Ib)-(If), (IIa)-(IIp), (IIb-1), (IIb-2), (IIc-1)-(IIc-2), (IIn-1), (IIn-2), (IVa)-(IV1), and (IXa)-(IXr)), each of R², R^{2′}, and R^{2″}, if present, is H. In further embodiments, each of R¹, R^{1′}, and R^{1″}, if present, is, independently, H, halo (e.g., fluoro), hydroxy, optionally substituted alkoxy (e.g., methoxy or ethoxy), or optionally substituted alkoxyalkoxy. In particular embodiments, alkoxyalkoxy is —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl). In some embodiments, s2 is 0, s1 is 1 or 2, s3 is 0 or 1, and R′ is C₁₋₆ alkyl.

In some embodiments of the nucleic acids or modified RNA (e.g., Formulas (Ia)-(Ia-5), (Ib)-(If-1), (IIa)-(IIp), (IIb-1), (IIb-2), (IIc-1)-(IIc-2), (IIn-1), (IIn-2), (IVa)-(IV1), and (IXa)-(IXr)), each of R³, R⁴, and R⁵ is, independently, H, halo (e.g., fluoro), hydroxy, optionally substituted alkyl, optionally substituted alkoxy (e.g., methoxy or ethoxy), or optionally substituted alkoxyalkoxy. In particular embodiments, R³ is H, R⁴ is H, R⁵ is H, or R³, R⁴, and R⁵ are all H. In particular embodiments, R³ is C₁₋₆ alkyl, R⁴ is C₁₋₆ alkyl, R⁵ is C₁₋₆ alkyl, or R³, R⁴, and R⁵ are all C₁₋₆ alkyl. In particular embodiments, R³ and R⁴ are both H, and R⁵ is C₁₋₆ alkyl.

In some embodiments of the nucleic acids or modified RNA (e.g., Formulas (Ia)-(Ia-5), (Ib)-(If-1), (IIa)-(IIp), (IIb-1), (IIb-2), (IIc-1)-(IIc-2), (IIn-1), (IIn-2), (IVa)-(IV1), and (IXa)-(IXr)), R³ and R⁵ join together to form optionally substituted alkylene or optionally substituted heteroalkylene and, taken together with the carbons to which they are attached, provide an optionally substituted heterocyclyl (e.g., a bicyclic, tricyclic, or tetracyclic heterocyclyl, such as trans-3′,4′ analogs, wherein R³ and R⁵ join together to form heteroalkylene (e.g., —(CH₂)_{b1}O(CH₂)_{b2}O(CH₂)_{b3}—, wherein each of b1, b2, and b3 are, independently, an integer from 0 to 3).

In some embodiments of the nucleic acids or modified RNA (e.g., Formulas (Ia)-(Ia-5), (Ib)-(If-1), (IIa)-(IIp), (IIb-1), (IIb-2), (IIc-1)-(IIc-2), (IIn-1), (IIn-2), (IVa)-(IV1), and (IXa)-(IXr)), R³ and one or more of R^{1′}, R^{1″}, R^{2′}, R^{2″}, or R⁵ join together to form optionally substituted alkylene or optionally substituted heteroalkylene and, taken together with the carbons to which they are attached, provide an optionally substituted heterocyclyl (e.g., a bicyclic, tricyclic, or tetracyclic heterocyclyl, R³ and one or more of R^{1′}, R^{1″}, R^{2′}, R^{2″}, or R⁵ join together to form heteroalkylene (e.g., —(CH₂)_{b1}O(CH₂)_{b2}O(CH₂)_{b3}—, wherein each of b1, b2, and b3 are, independently, an integer from 0 to 3).

In some embodiments of the nucleic acids or modified RNA (e.g., Formulas (Ia)-(Ia-5), (Ib)-(If-1), (IIa)-(IIp), (IIb-1), (IIb-2), (IIc-1)-(IIc-2), (IIn-1), (IIn-2), (IVa)-(IV1), and (IXa)-(IXr)), R⁵ and one or more of R^{1′}, R^{1″}, R^{2′}, or R^{2″} join together to form optionally substituted alkylene or optionally substituted heteroalkylene and, taken together with the carbons to which they are attached, provide an optionally substituted heterocyclyl (e.g., a bicyclic, tricyclic, or tetracyclic heterocyclyl, R⁵ and one or more of R^{1′}, R^{1″}, R^{2′}, or R^{2″} join together to form heteroalkylene (e.g., —(CH₂)_{b1}O(CH₂)_{b2}O(CH₂)_{b3}—, wherein each of b1, b2, and b3 are, independently, an integer from 0 to 3).

In some embodiments of the nucleic acids or modified RNA (e.g., Formulas (Ia)-(Ia-5), (Ib)-(If-1), (IIa)-(IIp), (IIb-1), (IIb-2), (IIc-1)-(IIc-2), (IIn-1), (IIn-2), (IVa)-(IV1), and (IXa)-(IXr)), each Y² is, independently, O, S, or —NR^{N1}—, wherein R^{N1} is H, optionally substituted alkyl, optionally substituted alkenyl, optionally substituted alkynyl, or optionally substituted aryl. In particular embodiments, Y² is NR^{N1}—, wherein R^{N1} is H or optionally substituted alkyl (e.g., C₁₋₆ alkyl, such as methyl, ethyl, isopropyl, or n-propyl).

In some embodiments of the nucleic acids or modified RNA (e.g., Formulas (Ia)-(Ia-5), (Ib)-(If-1), (IIa)-(IIp), (IIb-1), (IIb-2), (IIc-1)-(IIc-2), (IIn-1), (IIn-2), (IVa)-(IV1), and (IXa)-(IXr)), each Y³ is, independently, O or S.

In some embodiments of the nucleic acids or modified RNA (e.g., Formulas (Ia)-(Ia-5), (Ib)-(If-1), (IIa)-(IIp), (IIb-1), (IIb-2), (IIc-1)-(IIc-2), (IIn-1), (IIn-2), (IVa)-(IV1), and (IXa)-(IXr)), R¹ is H; each R² is, independently, H, halo (e.g., fluoro), hydroxy, optionally substituted alkoxy (e.g., methoxy or ethoxy), or optionally substituted alkoxyalkoxy (e.g., —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, such as wherein s2 is 0, s1 is 1 or 2, s3 is 0 or 1, and R′ is C₁₋₆ alkyl); each Y² is, independently, O or —NR^{N1}—, wherein R^{N1} is H, optionally substituted alkyl, optionally substituted alkenyl, optionally substituted alkynyl, or optionally substituted aryl (e.g., wherein R^{N1} is H or optionally substituted alkyl (e.g., C₁₋₆ alkyl, such as methyl, ethyl, isopropyl, or n-propyl)); and each Y³ is, independently, O or S (e.g., S). In further embodiments, R³ is H, halo (e.g., fluoro), hydroxy, optionally substituted alkyl, optionally substituted alkoxy (e.g., methoxy or ethoxy), or optionally substituted alkoxyalkoxy. In yet further embodiments, each Y¹ is, independently, O or —NR^{N1}—, wherein R^{N1} is H, optionally substituted alkyl, optionally substituted alkenyl, optionally substituted alkynyl, or optionally substituted aryl (e.g., wherein R^{N1} is H or optionally substituted alkyl (e.g., C₁₋₆ alkyl, such as methyl, ethyl, isopropyl, or n-propyl)); and each Y⁴ is, independently, H, hydroxy, thiol, optionally substituted alkyl, optionally substituted alkoxy, optionally substituted thioalkoxy, optionally substituted alkoxyalkoxy, or optionally substituted amino.

In some embodiments of the nucleic acids or modified RNA (e.g., Formulas (Ia)-(Ia-5), (Ib)-(If-1), (IIa)-(IIp), (IIb-1), (IIb-2), (IIc-1)-(IIc-2), (IIn-1), (IIn-2), (IVa)-(IV1), and (IXa)-(IXr)), each R¹ is, independently, H, halo (e.g., fluoro), hydroxy, optionally substituted alkoxy (e.g., methoxy or ethoxy), or optionally substituted alkoxyalkoxy (e.g., —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, such as wherein s2 is 0, s1 is 1 or 2, s3 is 0 or 1, and R′ is C₁₋₆ alkyl); R² is H; each Y² is, independently, O or —NR^{N1}—, wherein R^{N1} is H, optionally substituted alkyl, optionally substituted alkenyl, optionally substituted alkynyl, or optionally substituted aryl (e.g., wherein R^{N1} is H or optionally substituted alkyl (e.g., C₁₋₆ alkyl, such as methyl, ethyl, isopropyl, or n-propyl)); and each Y³ is, independently, O or S (e.g., S). In further embodiments, R³ is H, halo (e.g., fluoro), hydroxy, optionally substituted alkyl, optionally substituted alkoxy (e.g., methoxy or ethoxy), or optionally substituted alkoxyalkoxy. In yet further embodiments, each Y¹ is, independently, O or —NR^{N1}—, wherein R^{N1} is H, optionally substituted alkyl, optionally substituted alkenyl, optionally substituted alkynyl, or optionally substituted aryl (e.g., wherein R^{N1} is H or optionally substituted alkyl (e.g., C₁₋₆ alkyl, such as methyl, ethyl, isopropyl, or n-propyl)); and each Y⁴ is, independently, H, hydroxy, thiol, optionally substituted alkyl, optionally substituted alkoxy, optionally substituted thioalkoxy, optionally substituted alkoxyalkoxy, or optionally substituted amino.

In some embodiments of the nucleic acids or modified RNA (e.g., Formulas (Ia)-(Ia-5), (Ib)-(If-1), (IIa)-(IIp), (IIb-1), (IIb-2), (IIc-1)-(IIc-2), (IIn-1), (IIn-2), (IVa)-(IV1), and (IXa)-(IXr)), the ring including U is in the β-D (e.g., β-D-ribo) configuration.

In some embodiments of the polynucleotides (e.g., Formulas (Ia)-(Ia-5), (Ib)-(If-1), (IIa)-(IIp), (IIb-1), (IIb-2), (IIc-1)-(IIc-2), (IIn-1), (IIn-2), (IVa)-(IV1), and (IXa)-(IXr)), the ring including U is in the α-L (e.g., α-L-ribo) configuration.

In some embodiments of the nucleic acids or modified RNA (e.g., Formulas (Ia)-(Ia-5), (Ib)-(If-1), (IIa)-(IIp), (IIb-1), (IIb-2), (IIc-1)-(IIc-2), (IIn-1), (IIn-2), (IVa)-(IV1), and (IXa)-(IXr)), one or more B is not pseudouridine (ψ) or 5-methyl-cytidine (m⁵C).

In some embodiments, about 10% to about 100% of n number of B nucleobases is not w or m⁵C (e.g., from 10% to 20%, from 10% to 35%, from 10% to 50%, from 10% to 60%, from 10% to 75%, from 10% to 90%, from 10% to 95%, from 10% to 98%, from 10% to 99%, from 20% to 35%, from 20% to 50%, from 20% to 60%, from 20% to 75%, from 20% to 90%, from 20% to 95%, from 20% to 98%, from 20% to 99%, from 20% to 100%, from 50% to 60%, from 50% to 75%, from 50% to 90%, from 50% to 95%, from 50% to 98%, from 50% to 99%, from 50% to 100%, from 75% to 90%, from 75% to 95%, from 75% to 98%, from 75% to 99%, and from 75% to 100% of n number of B is not ψ or m⁵C). In some embodiments, B is not ψ or m⁵C.

In some embodiments of the polynucleotides (e.g., Formulas (Ia)-(Ia-5), (Ib)-(If-1), (IIa)-(IIp), (IIb-1), (IIb-2), (IIc-1)-(IIc-2), (IIn-1), (IIn-2), (IVa)-(IV1), and (IXa)-(IXr)), when B is an unmodified nucleobase selected from cytosine, guanine, uracil and adenine, then at least one of Y¹, Y², or Y³ is not O.

In some embodiments, the nucleic acids or modified RNA includes a modified ribose. In some embodiments, the polynucleotide (e.g., the first region, the first flanking region, or the second flanking region) includes n number of linked nucleosides having Formula (IIa)-(IIc):

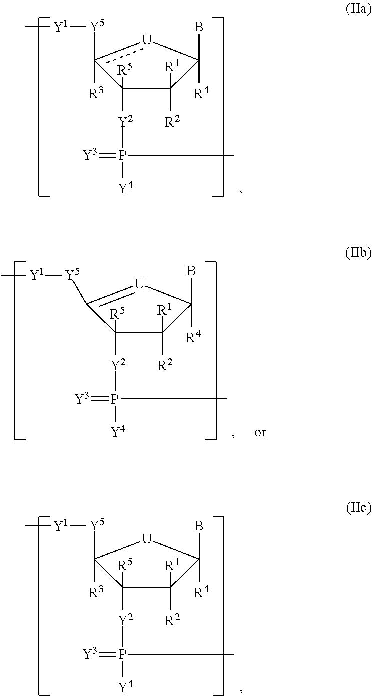

or a pharmaceutically acceptable salt or stereoisomer thereof. In particular embodiments, U is O or C(R^{U})ₙᵤ, wherein nu is an integer from 0 to 2 and each R^{U} is, independently, H, halo, or optionally substituted alkyl (e.g., U is —CH₂— or —CH—). In other embodiments, each of R¹, R², R³, R⁴, and R⁵ is, independently, H, halo, hydroxy, thiol, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted hydroxyalkoxy, optionally substituted amino, azido, optionally substituted aryl, optionally substituted aminoalkyl, optionally substituted aminoalkenyl, optionally substituted aminoalkynyl, or absent (e.g., each R¹ and R² is, independently H, halo, hydroxy, optionally substituted alkyl, or optionally substituted alkoxy; each R³ and R⁴ is, independently, H or optionally substituted alkyl; and R⁵ is H or hydroxy), and [symbol] is a single bond or double bond.

In particular embodiments, the nucleic acids or modified RNA (e.g., the first region, the first flanking region, or the second flanking region) includes n number of linked nucleosides having Formula (IIb-1)-(IIb-2):

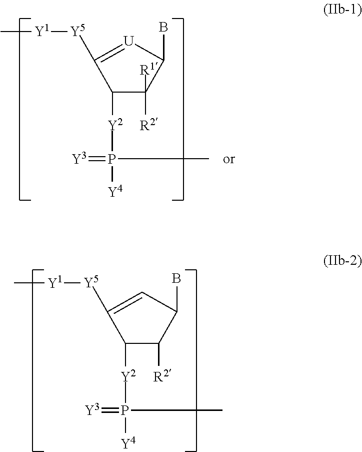

or a pharmaceutically acceptable salt or stereoisomer thereof. In some embodiments, U is O or C(R^{U})ₙᵤ, wherein nu is an integer from 0 to 2 and each R^{U} is, independently, H, halo, or optionally substituted alkyl (e.g., U is —CH₂— or —CH—). In other embodiments, each of R¹ and R² is, independently, H, halo, hydroxy, thiol, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted hydroxyalkoxy, optionally substituted amino, azido, optionally substituted aryl, optionally substituted aminoalkyl, optionally substituted aminoalkenyl, optionally substituted aminoalkynyl, or absent (e.g., each R¹ and R² is, independently, H, halo, hydroxy, optionally substituted alkyl, or optionally substituted alkoxy, e.g., H, halo, hydroxy, alkyl, or alkoxy). In particular embodiments, R² is hydroxy or optionally substituted alkoxy (e.g., methoxy, ethoxy, or any described herein).

In particular embodiments, the nucleic acids or modified RNA (e.g., the first region, the first flanking region, or the second flanking region) includes n number of linked nucleosides having Formula (IIc-1)-(IIc-4):

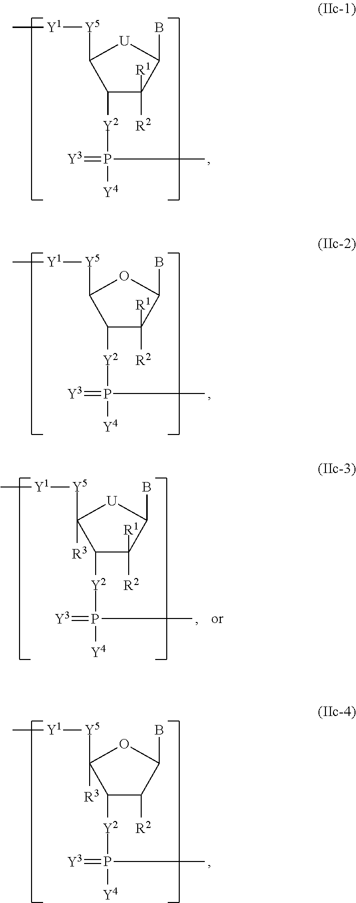

or a pharmaceutically acceptable salt or stereoisomer thereof.

In some embodiments, U is O or C(R^{U})ₙᵤ, wherein nu is an integer from 0 to 2 and each R^{U} is, independently, H, halo, or optionally substituted alkyl (e.g., U is —CH₂— or —CH—). In some embodiments, each of R², and R³ is, independently, H, halo, hydroxy, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted hydroxyalkoxy, optionally substituted amino, azido, optionally substituted aryl, optionally substituted aminoalkyl, optionally substituted aminoalkenyl, optionally substituted aminoalkynyl, or absent (e.g., each R¹ and R² is, independently, H, halo, hydroxy, optionally substituted alkyl, or optionally substituted alkoxy, e.g., H, halo, hydroxy, alkyl, or alkoxy; and each R³ is, independently, H or optionally substituted alkyl)). In particular embodiments, R² is optionally substituted alkoxy (e.g., methoxy or ethoxy, or any described herein). In particular embodiments, le is optionally substituted alkyl, and R² is hydroxy. In other embodiments, le is hydroxy, and R² is optionally substituted alkyl. In further embodiments, R³ is optionally substituted alkyl.

In some embodiments, the nucleic acids or modified RNA includes an acyclic modified ribose. In some embodiments, the polynucleotide (e.g., the first region, the first flanking region, or the second flanking region) includes n number of linked nucleosides having Formula (IId)-(IIf):

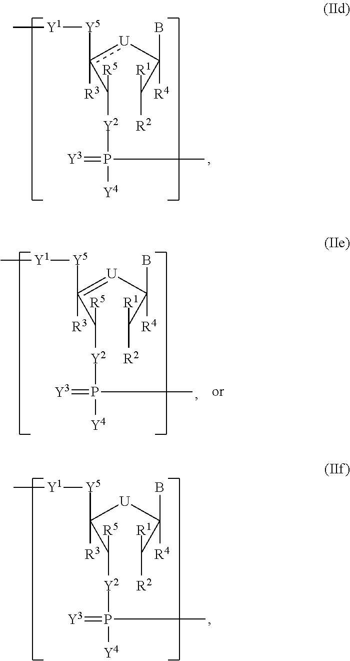

or a pharmaceutically acceptable salt or stereoisomer thereof.

In some embodiments, the nucleic acids or modified RNA includes an acyclic modified hexitol. In some embodiments, the polynucleotide (e.g., the first region, the first flanking region, or the second flanking region) includes n number of linked nucleosides having Formula (IIg)-(IIj):

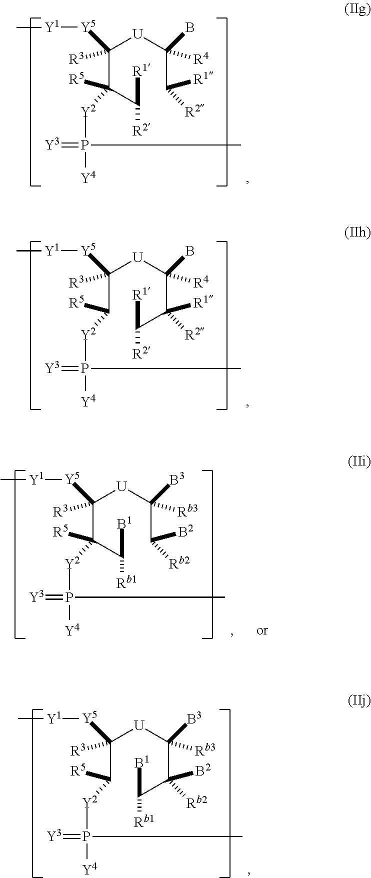

or a pharmaceutically acceptable salt or stereoisomer thereof.

In some embodiments, the nucleic acids or modified RNA includes a sugar moiety having a contracted or an expanded ribose ring. In some embodiments, the polynucleotide (e.g., the first region, the first flanking region, or the second flanking region) includes n number of linked nucleosides having Formula (IIk)-(IIm):

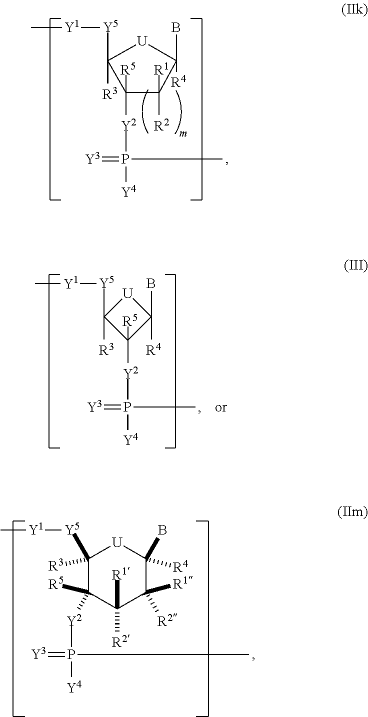

or a pharmaceutically acceptable salt or stereoisomer thereof, wherein each of R^{1′}, R^{1″}, R^{2′}, and R^{2″} is, independently, H, halo, hydroxy, optionally substituted alkyl, optionally substituted alkoxy, optionally substituted alkenyloxy, optionally substituted alkynyloxy, optionally substituted aminoalkoxy, optionally substituted alkoxyalkoxy, or absent; and wherein the combination of R^{2′} and R³ or the combination of R^{2″} and R³ can be taken together to form optionally substituted alkylene or optionally substituted heteroalkylene.

In some embodiments, the nucleic acids or modified RNA includes a locked modified ribose. In some embodiments, the polynucleotide (e.g., the first region, the first flanking region, or the second flanking region) includes n number of linked nucleosides having Formula (IIn):

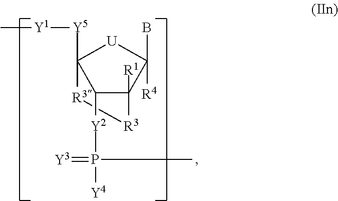

or a pharmaceutically acceptable salt or stereoisomer thereof, wherein R^{3′} is O, S, or —NR^{N1}—, wherein R^{N1} is H, optionally substituted alkyl, optionally substituted alkenyl, optionally substituted alkynyl, or optionally substituted aryl and R^{3″} is optionally substituted alkylene (e.g., —CH₂—, —CH₂CH₂—, or —CH₂CH₂CH₂—) or optionally substituted heteroalkylene (e.g., —CH₂NH—, —CH₂CH₂NH—, —CH₂OCH₂—, or —CH₂CH₂OCH₂—) (e.g., R^{3′} is O and R³″ is optionally substituted alkylene (e.g., —CH₂—, —CH₂CH₂—, or —CH₂CH₂CH₂—)).

In some embodiments, the nucleic acids or modified RNA (e.g., the first region, the first flanking region, or the second flanking region) includes n number of linked nucleosides having Formula (IIn-1)-(II-n2):

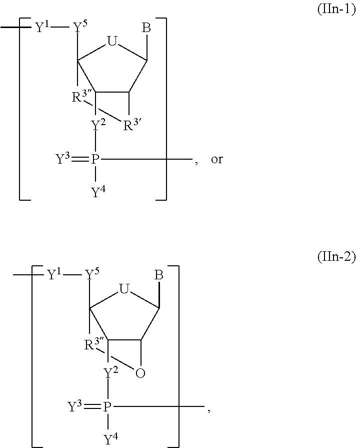

or a pharmaceutically acceptable salt or stereoisomer thereof, wherein R^{3′} is O, S, or —NR^{N1}—, wherein R^{N1} is H, optionally substituted alkyl, optionally substituted alkenyl, optionally substituted alkynyl, or optionally substituted aryl and R^{3″} is optionally substituted alkylene (e.g., —CH₂—, —CH₂CH₂—, or —CH₂CH₂CH₂—) or optionally substituted heteroalkylene (e.g., —CH₂NH—, —CH₂CH₂NH—, —CH₂OCH₂—, or —CH₂CH₂OCH₂—) (e.g., R^{3′} is O and R^{3″} is optionally substituted alkylene (e.g., —CH₂—, —CH₂CH₂—, or —CH₂CH₂CH₂—)).

In some embodiments, the nucleic acids or modified RNA includes a locked modified ribose that forms a tetracyclic heterocyclyl. In some embodiments, the nucleic acids or modified RNA (e.g., the first region, the first flanking region, or the second flanking region) includes n number of linked nucleosides having Formula (IIo):

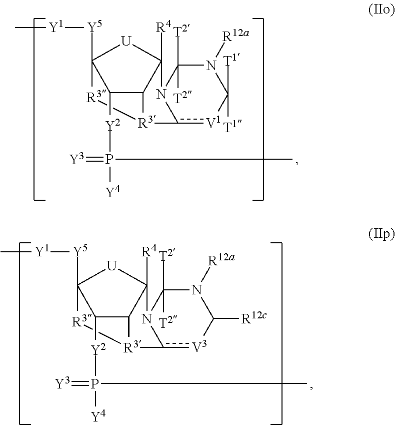

or a pharmaceutically acceptable salt or stereoisomer thereof, wherein R^{12a}, R^{12c}, T^{1′}, T^{1″}, T^{2′}, T^{2″}, V¹, and V³ are as described herein.

Any of the formulas for the nucleic acids or modified RNA can include one or more nucleobases described herein (e.g., Formulas (b1)-(b43)).

In one embodiment, the present invention provides methods of preparing a nucleic acids or modified RNA comprising at least one nucleotide wherein the polynucleotide comprises n number of nucleosides having Formula (Ia), as defined herein:

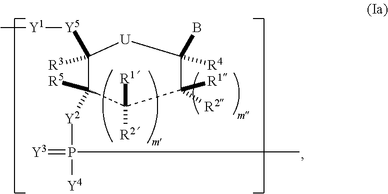

the method comprising reacting a compound of Formula (IIIa), as defined herein:

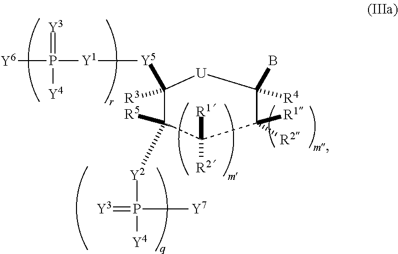

with an RNA polymerase, and a cDNA template.

In a further embodiment, the present invention provides methods of amplifying a nucleic acids or modified RNA comprising: reacting a compound of Formula (IIIa), as defined herein, with a primer, a cDNA template, and an RNA polymerase.

In one embodiment, the present invention provides methods of preparing a nucleic acids or modified RNA comprising at least one nucleotide, wherein the nucleic acids or modified RNA comprises n number of nucleosides having Formula (Ia-1), as defined herein:

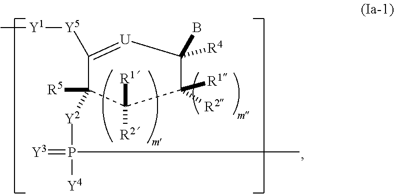

the method comprising reacting a compound of Formula (IIIa-1), as defined herein:

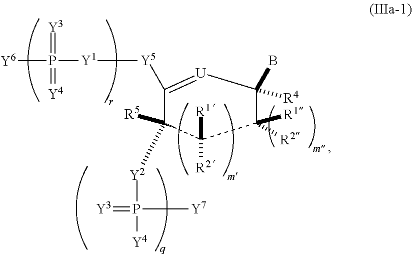

with an RNA polymerase, and a cDNA template.

In a further embodiment, the present invention provides methods of amplifying a nucleic acids or modified RNA comprising at least one nucleotide (e.g., modified mRNA molecule), the method comprising: reacting a compound of Formula (IIIa-1), as defined herein, with a primer, a cDNA template, and an RNA polymerase.

In one embodiment, the present invention provides methods of preparing a nucleic acids or modified RNA comprising at least one nucleotide, wherein the nucleic acids or modified RNA comprises n number of nucleosides having Formula (Ia-2), as defined herein:

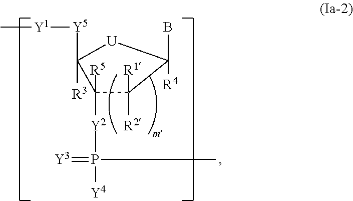

the method comprising reacting a compound of Formula (IIIa-2), as defined herein:

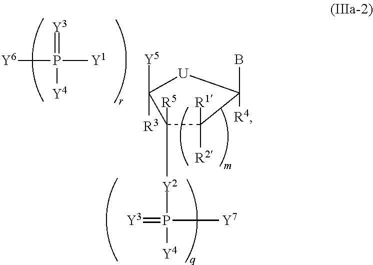

with an RNA polymerase, and a cDNA template.

In a further embodiment, the present invention provides methods of amplifying a nucleic acids or modified RNA comprising at least one nucleotide (e.g., modified mRNA molecule), the method comprising reacting a compound of Formula (IIIa-2), as defined herein, with a primer, a cDNA template, and an RNA polymerase.

In some embodiments, the reaction may be repeated from 1 to about 7,000 times. In any of the embodiments herein, B may be a nucleobase of Formula (b1)-(b43).

The nucleic acids or modified RNA can optionally include 5′ and/or 3′ flanking regions, which are described herein.

### Major Groove Interacting Partners

As described herein, the phrase “major groove interacting partner” refers RNA recognition receptors that detect and respond to RNA ligands through interactions, e.g. binding, with the major groove face of a nucleotide or nucleic acid. As such, RNA ligands comprising modified nucleotides or nucleic acids as described herein decrease interactions with major groove binding partners, and therefore decrease an innate immune response.

Example major groove interacting, e.g. binding, partners include, but are not limited to the following nucleases and helicases. Within membranes, TLRs (Toll-like Receptors) 3, 7, and 8 can respond to single- and double-stranded RNAs. Within the cytoplasm, members of the superfamily 2 class of DEX(D/H) helicases and ATPases can sense RNAs to initiate antiviral responses. These helicases include the RIG-I (retinoic acid-inducible gene I) and MDA5 (melanoma differentiation-associated gene 5). Other examples include laboratory of genetics and physiology 2 (LGP2), HIN-200 domain containing proteins, or Helicase-domain containing proteins.

### Prevention or Reduction of Innate Cellular Immune Response Activation Using Modified Nucleic Acids

The term “innate immune response” includes a cellular response to exogenous nucleic acids, including single stranded nucleic acids, generally of viral or bacterial origin, which involves the induction of cytokine expression and release, particularly the interferons, and cell death. Protein synthesis is also reduced during the innate cellular immune response. While it is advantageous to eliminate the innate immune response in a cell, the present disclosure provides modified mRNAs that substantially reduce the immune response, including interferon signaling, without entirely eliminating such a response. In some embodiments, the immune response is reduced by 10%, 20%, 30%, 40%, 50%, 60%, 70%, 80%, 90%, 95%, 99%, 99.9%, or greater than 99.9% as compared to the immune response induced by a corresponding unmodified nucleic acid. Such a reduction can be measured by expression or activity level of Type 1 interferons or the expression of interferon-regulated genes such as the toll-like receptors (e.g., TLR7 and TLR8). Reduction of innate immune response can also be measured by decreased cell death following one or more administrations of modified RNAs to a cell population; e.g., cell death is 10%, 25%, 50%, 75%, 85%, 90%, 95%, or over 95% less than the cell death frequency observed with a corresponding unmodified nucleic acid. Moreover, cell death may affect fewer than 50%, 40%, 30%, 20%, 10%, 5%, 1%, 0.1%, 0.01% or fewer than 0.01% of cells contacted with the modified nucleic acids.

The present disclosure provides for the repeated introduction (e.g., transfection) of modified nucleic acids into a target cell population, e.g., in vitro, ex vivo, or in vivo. The step of contacting the cell population may be repeated one or more times (such as two, three, four, five or more than five times). In some embodiments, the step of contacting the cell population with the modified nucleic acids is repeated a number of times sufficient such that a predetermined efficiency of protein translation in the cell population is achieved. Given the reduced cytotoxicity of the target cell population provided by the nucleic acid modifications, such repeated transfections are achievable in a diverse array of cell types.

### Polypeptide Variants

Provided are nucleic acids that encode variant polypeptides, which have a certain identity with a reference polypeptide sequence. The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

In some embodiments, the polypeptide variant has the same or a similar activity as the reference polypeptide. Alternatively, the variant has an altered activity (e.g., increased or decreased) relative to a reference polypeptide. Generally, variants of a particular polynucleotide or polypeptide of the present disclosure will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular reference polynucleotide or polypeptide as determined by sequence alignment programs and parameters described herein and known to those skilled in the art.

As recognized by those skilled in the art, protein fragments, functional protein domains, and homologous proteins are also considered to be within the scope of this present disclosure. For example, provided herein is any protein fragment of a reference protein (meaning a polypeptide sequence at least one amino acid residue shorter than a reference polypeptide sequence but otherwise identical) 10, 20, 30, 40, 50, 60, 70, 80, 90, 100 or greater than 100 amino acids in length In another example, any protein that includes a stretch of about 20, about 30, about 40, about 50, or about 100 amino acids which are about 40%, about 50%, about 60%, about 70%, about 80%, about 90%, about 95%, or about 100% identical to any of the sequences described herein can be utilized in accordance with the present disclosure. In certain embodiments, a protein sequence to be utilized in accordance with the present disclosure includes 2, 3, 4, 5, 6, 7, 8, 9, 10, or more mutations as shown in any of the sequences provided or referenced herein.

### Polypeptide Libraries

Also provided are polynucleotide libraries containing nucleoside modifications, wherein the polynucleotides individually contain a first nucleic acid sequence encoding a polypeptide, such as an antibody, protein binding partner, scaffold protein, and other polypeptides known in the art. Preferably, the polynucleotides are mRNA in a form suitable for direct introduction into a target cell host, which in turn synthesizes the encoded polypeptide.

In certain embodiments, multiple variants of a protein, each with different amino acid modification(s), are produced and tested to determine the best variant in terms of pharmacokinetics, stability, biocompatibility, and/or biological activity, or a biophysical property such as expression level. Such a library may contain 10, 10², 10³, 10⁴, 10⁵, 10⁶, 10⁷, 10⁸, 10⁹, or over 10⁹ possible variants (including substitutions, deletions of one or more residues, and insertion of one or more residues).

### Polypeptide-Nucleic Acid Complexes

Proper protein translation involves the physical aggregation of a number of polypeptides and nucleic acids associated with the mRNA. Provided by the present disclosure are protein-nucleic acid complexes, containing a translatable mRNA having one or more nucleoside modifications (e.g., at least two different nucleoside modifications) and one or more polypeptides bound to the mRNA. Generally, the proteins are provided in an amount effective to prevent or reduce an innate immune response of a cell into which the complex is introduced.

### Untranslatable Modified Nucleic Acids

As described herein, provided are mRNAs having sequences that are substantially not translatable. Such mRNA is effective as a vaccine when administered to a mammalian subject.

Also provided are modified nucleic acids that contain one or more noncoding regions. Such modified nucleic acids are generally not translated, but are capable of binding to and sequestering one or more translational machinery component such as a ribosomal protein or a transfer RNA (tRNA), thereby effectively reducing protein expression in the cell. The modified nucleic acid may contain a small nucleolar RNA (sno-RNA), micro RNA (miRNA), small interfering RNA (siRNA) or Piwi-interacting RNA (piRNA).

### Synthesis of Modified Nucleic Acids

Nucleic acids for use in accordance with the present disclosure may be prepared according to any available technique including, but not limited to chemical synthesis, enzymatic synthesis, which is generally termed in vitro transcription, enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference in their entirety).

The modified nucleosides and nucleotides disclosed herein can be prepared from readily available starting materials using the following general methods and procedures. It is understood that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

Preparation of modified nucleosides and nucleotides can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents, which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Resolution of racemic mixtures of modified nucleosides and nucleotides can be carried out by any of numerous methods known in the art. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Resolution of racemic mixtures can also be carried out by elution on a column packed with an optically active resolving agent (e.g., dinitrobenzoylphenylglycine). Suitable elution solvent composition can be determined by one skilled in the art. Modified nucleic acids need not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of a nucleic acid such that the function of the nucleic acid is not substantially decreased. A modification may also be a 5′ or 3′ terminal modification. The nucleic acids may contain at a minimum one and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as uracil or cytosine. In some embodiments, at least 5%, at least 10%, at least 25%, at least 50%, at least 80%, at least 90% or 100% of the uracil in the nucleic acid is replaced with a modified uracil. The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine. The modified cytosine can be replaced by a compound having a single unique structure, or can be replaced by a plurality of compounds having different structures (e.g., 2, 3, 4 or more unique structures).

Generally, the shortest length of a modified mRNA of the present disclosure can be the length of an mRNA sequence that is sufficient to encode for a dipeptide. In another embodiment, the length of the mRNA sequence is sufficient to encode for a tripeptide. In another embodiment, the length of an mRNA sequence is sufficient to encode for a tetrapeptide. In another embodiment, the length of an mRNA sequence is sufficient to encode for a pentapeptide. In another embodiment, the length of an mRNA sequence is sufficient to encode for a hexapeptide. In another embodiment, the length of an mRNA sequence is sufficient to encode for a heptapeptide. In another embodiment, the length of an mRNA sequence is sufficient to encode for an octapeptide. In another embodiment, the length of an mRNA sequence is sufficient to encode for a nonapeptide. In another embodiment, the length of an mRNA sequence is sufficient to encode for a decapeptide.

Examples of dipeptides that the modified nucleic acid sequences can encode for include, but are not limited to, carnosine and anserine.

In a further embodiment, the mRNA is greater than 30 nucleotides in length. In another embodiment, the RNA molecule is greater than 35 nucleotides in length. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 60 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1800 nucleotides. In another embodiment, the length is at least 2000 nucleotides. In another embodiment, the length is at least 2500 nucleotides. In another embodiment, the length is at least 3000 nucleotides. In another embodiment, the length is at least 4000 nucleotides. In another embodiment, the length is at least 5000 nucleotides, or greater than 5000 nucleotides.

### Uses of Modified Nucleic Acids

### Therapeutic Agents

The modified nucleic acids and the proteins translated from the modified nucleic acids described herein can be used as therapeutic agents. For example, a modified nucleic acid described herein can be administered to a subject, wherein the modified nucleic acid is translated in vivo to produce a therapeutic peptide in the subject. Accordingly, provided herein are compositions, methods, kits, and reagents for treatment or prevention of disease or conditions in humans and other mammals. The active therapeutic agents of the present disclosure include modified nucleic acids, cells containing modified nucleic acids or polypeptides translated from the modified nucleic acids, polypeptides translated from modified nucleic acids, and cells contacted with cells containing modified nucleic acids or polypeptides translated from the modified nucleic acids.

In certain embodiments, provided are combination therapeutics containing one or more modified nucleic acids containing translatable regions that encode for a protein or proteins that boost a mammalian subject's immunity along with a protein that induces antibody-dependent cellular toxicity. For example, provided are therapeutics containing one or more nucleic acids that encode trastuzumab and granulocyte-colony stimulating factor (G-CSF). In particular, such combination therapeutics are useful in Her2+ breast cancer patients who develop induced resistance to trastuzumab. (See, e.g., Albrecht, Immunotherapy. 2(6):795-8 (2010)).

Provided are methods of inducing translation of a recombinant polypeptide in a cell population using the modified nucleic acids described herein. Such translation can be in vivo, ex vivo, in culture, or in vitro. The cell population is contacted with an effective amount of a composition containing a nucleic acid that has at least one nucleoside modification, and a translatable region encoding the recombinant polypeptide. The population is contacted under conditions such that the nucleic acid is localized into one or more cells of the cell population and the recombinant polypeptide is translated in the cell from the nucleic acid.

An effective amount of the composition is provided based, at least in part, on the target tissue, target cell type, means of administration, physical characteristics of the nucleic acid (e.g., size, and extent of modified nucleosides), and other determinants. In general, an effective amount of the composition provides efficient protein production in the cell, preferably more efficient than a composition containing a corresponding unmodified nucleic acid. Increased efficiency may be demonstrated by increased cell transfection (i.e., the percentage of cells transfected with the nucleic acid), increased protein translation from the nucleic acid, decreased nucleic acid degradation (as demonstrated, e.g., by increased duration of protein translation from a modified nucleic acid), or reduced innate immune response of the host cell.

Aspects of the present disclosure are directed to methods of inducing in vivo translation of a recombinant polypeptide in a mammalian subject in need thereof. Therein, an effective amount of a composition containing a nucleic acid that has at least one nucleoside modification and a translatable region encoding the recombinant polypeptide is administered to the subject using the delivery methods described herein. The nucleic acid is provided in an amount and under other conditions such that the nucleic acid is localized into a cell of the subject and the recombinant polypeptide is translated in the cell from the nucleic acid. The cell in which the nucleic acid is localized, or the tissue in which the cell is present, may be targeted with one or more than one rounds of nucleic acid administration.

Other aspects of the present disclosure relate to transplantation of cells containing modified nucleic acids to a mammalian subject. Administration of cells to mammalian subjects is known to those of ordinary skill in the art, such as local implantation (e.g., topical or subcutaneous administration), organ delivery or systemic injection (e.g., intravenous injection or inhalation), as is the formulation of cells in pharmaceutically acceptable carrier. Compositions containing modified nucleic acids are formulated for administration intramuscularly, transarterially, intraperitoneally, intravenously, intranasally, subcutaneously, endoscopically, transdermally, or intrathecally. In some embodiments, the composition is formulated for extended release.

The subject to whom the therapeutic agent is administered suffers from or is at risk of developing a disease, disorder, or deleterious condition. Provided are methods of identifying, diagnosing, and classifying subjects on these bases, which may include clinical diagnosis, biomarker levels, genome-wide association studies (GWAS), and other methods known in the art.

In certain embodiments, the administered modified nucleic acid directs production of one or more recombinant polypeptides that provide a functional activity which is substantially absent in the cell in which the recombinant polypeptide is translated. For example, the missing functional activity may be enzymatic, structural, or gene regulatory in nature.

In other embodiments, the administered modified nucleic acid directs production of one or more recombinant polypeptides that replace a polypeptide (or multiple polypeptides) that is substantially absent in the cell in which the recombinant polypeptide is translated. Such absence may be due to genetic mutation of the encoding gene or regulatory pathway thereof. Alternatively, the recombinant polypeptide functions to antagonize the activity of an endogenous protein present in, on the surface of, or secreted from the cell. Usually, the activity of the endogenous protein is deleterious to the subject, for example, do to mutation of the endogenous protein resulting in altered activity or localization. Additionally, the recombinant polypeptide antagonizes, directly or indirectly, the activity of a biological moiety present in, on the surface of, or secreted from the cell. Examples of antagonized biological moieties include lipids (e.g., cholesterol), a lipoprotein (e.g., low density lipoprotein), a nucleic acid, a carbohydrate, or a small molecule toxin.

The recombinant proteins described herein are engineered for localization within the cell, potentially within a specific compartment such as the nucleus, or are engineered for secretion from the cell or translocation to the plasma membrane of the cell.

As described herein, a useful feature of the modified nucleic acids of the present disclosure is the capacity to reduce the innate immune response of a cell to an exogenous nucleic acid. Provided are methods for performing the titration, reduction or elimination of the immune response in a cell or a population of cells. In some embodiments, the cell is contacted with a first composition that contains a first dose of a first exogenous nucleic acid including a translatable region and at least one nucleoside modification, and the level of the innate immune response of the cell to the first exogenous nucleic acid is determined. Subsequently, the cell is contacted with a second composition, which includes a second dose of the first exogenous nucleic acid, the second dose containing a lesser amount of the first exogenous nucleic acid as compared to the first dose. Alternatively, the cell is contacted with a first dose of a second exogenous nucleic acid. The second exogenous nucleic acid may contain one or more modified nucleosides, which may be the same or different from the first exogenous nucleic acid or, alternatively, the second exogenous nucleic acid may not contain modified nucleosides. The steps of contacting the cell with the first composition and/or the second composition may be repeated one or more times. Additionally, efficiency of protein production (e.g., protein translation) in the cell is optionally determined, and the cell may be re-transfected with the first and/or second composition repeatedly until a target protein production efficiency is achieved.

### Therapeutics for Diseases and Conditions

Provided are methods for treating or preventing a symptom of diseases characterized by missing or aberrant protein activity, by replacing the missing protein activity or overcoming the aberrant protein activity. Because of the rapid initiation of protein production following introduction of modified mRNAs, as compared to viral DNA vectors, the compounds of the present disclosure are particularly advantageous in treating acute diseases such as sepsis, stroke, and myocardial infarction. Moreover, the lack of transcriptional regulation of the modified mRNAs of the present disclosure is advantageous in that accurate titration of protein production is achievable.

Diseases characterized by dysfunctional or aberrant protein activity include, but not limited to, cancer and proliferative diseases, genetic diseases (e.g., cystic fibrosis), autoimmune diseases, diabetes, neurodegenerative diseases, cardiovascular diseases, and metabolic diseases. The present disclosure provides a method for treating such conditions or diseases in a subject by introducing nucleic acid or cell-based therapeutics containing the modified nucleic acids provided herein, wherein the modified nucleic acids encode for a protein that antagonizes or otherwise overcomes the aberrant protein activity present in the cell of the subject. Specific examples of a dysfunctional protein are the missense mutation variants of the cystic fibrosis transmembrane conductance regulator (CFTR) gene, which produce a dysfunctional protein variant of CFTR protein, which causes cystic fibrosis.

Multiple diseases are characterized by missing (or substantially diminished such that proper protein function does not occur) protein activity. Such proteins may not be present, or are essentially non-functional. The present disclosure provides a method for treating such conditions or diseases in a subject by introducing nucleic acid or cell-based therapeutics containing the modified nucleic acids provided herein, wherein the modified nucleic acids encode for a protein that replaces the protein activity missing from the target cells of the subject. Specific examples of a dysfunctional protein are the nonsense mutation variants of the cystic fibrosis transmembrane conductance regulator (CFTR) gene, which produce a nonfunctional protein variant of CFTR protein, which causes cystic fibrosis.

Thus, provided are methods of treating cystic fibrosis in a mammalian subject by contacting a cell of the subject with a modified nucleic acid having a translatable region that encodes a functional CFTR polypeptide, under conditions such that an effective amount of the CTFR polypeptide is present in the cell. Preferred target cells are epithelial cells, such as the lung, and methods of administration are determined in view of the target tissue; i.e., for lung delivery, the RNA molecules are formulated for administration by inhalation.

In another embodiment, the present disclosure provides a method for treating hyperlipidemia in a subject, by introducing into a cell population of the subject with a modified mRNA molecule encoding Sortilin, a protein recently characterized by genomic studies, thereby ameliorating the hyperlipidemia in a subject. The SORT1 gene encodes a trans-Golgi network (TGN) transmembrane protein called Sortilin. Genetic studies have shown that one of five individuals has a single nucleotide polymorphism, rs12740374, in the 1p13 locus of the SORT1 gene that predisposes them to having low levels of low-density lipoprotein (LDL) and very-low-density lipoprotein (VLDL). Each copy of the minor allele, present in about 30% of people, alters LDL cholesterol by 8 mg/dL, while two copies of the minor allele, present in about 5% of the population, lowers LDL cholesterol 16 mg/dL. Carriers of the minor allele have also been shown to have a 40% decreased risk of myocardial infarction. Functional in vivo studies in mice describes that overexpression of SORT1 in mouse liver tissue led to significantly lower LDL-cholesterol levels, as much as 80% lower, and that silencing SORT1 increased LDL cholesterol approximately 200% (Musunuru K et al. From noncoding variant to phenotype via SORT1 at the 1p13 cholesterol locus. Nature 2010; 466: 714-721).

### Methods of Cellular Nucleic Acid Delivery

Methods of the present disclosure enhance nucleic acid delivery into a cell population, in vivo, ex vivo, or in culture. For example, a cell culture containing a plurality of host cells (e.g., eukaryotic cells such as yeast or mammalian cells) is contacted with a composition that contains an enhanced nucleic acid having at least one nucleoside modification and, optionally, a translatable region. The composition also generally contains a transfection reagent or other compound that increases the efficiency of enhanced nucleic acid uptake into the host cells. The enhanced nucleic acid exhibits enhanced retention in the cell population, relative to a corresponding unmodified nucleic acid. The retention of the enhanced nucleic acid is greater than the retention of the unmodified nucleic acid. In some embodiments, it is at least about 50%, 75%, 90%, 95%, 100%, 150%, 200% or more than 200% greater than the retention of the unmodified nucleic acid. Such retention advantage may be achieved by one round of transfection with the enhanced nucleic acid, or may be obtained following repeated rounds of transfection.

In some embodiments, the enhanced nucleic acid is delivered to a target cell population with one or more additional nucleic acids. Such delivery may be at the same time, or the enhanced nucleic acid is delivered prior to delivery of the one or more additional nucleic acids. The additional one or more nucleic acids may be modified nucleic acids or unmodified nucleic acids. It is understood that the initial presence of the enhanced nucleic acids does not substantially induce an innate immune response of the cell population and, moreover, that the innate immune response will not be activated by the later presence of the unmodified nucleic acids. In this regard, the enhanced nucleic acid may not itself contain a translatable region, if the protein desired to be present in the target cell population is translated from the unmodified nucleic acids.

### Targeting Moieties

In some embodiments, modified nucleic acids are provided to express a protein-binding partner or a receptor on the surface of the cell, which functions to target the cell to a specific tissue space or to interact with a specific moiety, either in vivo or in vitro. Suitable protein-binding partners include antibodies and functional fragments thereof, scaffold proteins, or peptides. Additionally, modified nucleic acids can be employed to direct the synthesis and extracellular localization of lipids, carbohydrates, or other biological moieties.

### Permanent Gene Expression Silencing

A method for epigenetically silencing gene expression in a mammalian subject, comprising a nucleic acid where the translatable region encodes a polypeptide or polypeptides capable of directing sequence-specific histone H3 methylation to initiate heterochromatin formation and reduce gene transcription around specific genes for the purpose of silencing the gene. For example, a gain-of-function mutation in the Janus Kinase 2 gene is responsible for the family of Myeloproliferative Diseases.

### Pharmaceutical Compositions

### Formulation, Administration, Delivery and Dosing

The present disclosure provides proteins generated from modified mRNAs. Pharmaceutical compositions may optionally comprise one or more additional therapeutically active substances. In accordance with some embodiments, a method of administering pharmaceutical compositions comprising one or more proteins to be delivered to a subject in need thereof is provided. In some embodiments, compositions are administered to humans. For the purposes of the present disclosure, the phrase “active ingredient” generally refers to a modified nucleic acid, a protein or a protein-containing complex as described herein.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure will vary, depending upon the identity, size, and/or condition of the subject treated and further depending upon the route by which the composition is to be administered. By way of example, the composition may comprise between 0.1% and 100% (w/w) active ingredient.

### Formulations

The modified nucleic acid of the invention can be formulated using one or more excipients to: (1) increase stability; (2) increase cell transfection; (3) permit the sustained or delayed release (e.g., from a depot formulation of the modified nucleic acids); (4) alter the biodistribution (e.g., target the modified nucleic acids to specific tissues or cell types); (5) increase the translation of encoded protein in vivo; and/or (6) alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with modified nucleic acid (e.g., for transplantation into a subject), hyaluronidase, nanoparticle mimics and combinations thereof. Accordingly, the formulations of the invention can include one or more excipients, each in an amount that together increases the stability of the modified nucleic acid increases cell transfection by the modified nucleic acid increases the expression of modified nucleic acid encoded protein, and/or alters the release profile of modified nucleic acid encoded proteins. Further, the modified nucleic acid of the present invention may be formulated using self-assembled nucleic acid nanoparticles.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient may generally be equal to the dosage of the active ingredient which would be administered to a subject and/or a convenient fraction of such a dosage including, but not limited to, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient.

In some embodiments, the modified mRNA formulations described herein may contain at least one modified mRNA. The formulations may contain 1, 2, 3, 4 or 5 modified mRNA. In one embodiment, the formulation contains at least three modified mRNA encoding proteins. In one embodiment, the formulation contains at least five modified mRNA encoding proteins.

Pharmaceutical formulations may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

In some embodiments, the particle size of the lipid nanoparticle may be increased and/or decreased. The change in particle size may be able to help counter biological reaction such as, but not limited to, inflammation or may increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients may optionally be included in the pharmaceutical formulations of the invention

### Lipidoid

The synthesis of lipidoids has been extensively described and formulations containing these compounds are particularly suited for delivery of modified nucleic acids (see Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001; all of which are incorporated herein by reference in their entireties).

While these lipidoids have been used to effectively deliver double stranded small interfering RNA molecules in rodents and non-human primates (see Akinc et al., Nat Biotechnol. 2008 26:561-569; Frank-Kamenetsky et al., Proc Natl Acad Sci USA. 2008 105:11915-11920; Akinc et al., Mol Ther. 2009 17:872-879; Love et al., Proc Natl Acad Sci USA. 2010 107:1864-1869; Leuschner et al., Nat Biotechnol. 2011 29:1005-1010; all of which is incorporated herein by reference in their entirety), the present disclosure describes their formulation and use in delivering single stranded modified nucleic acids. Complexes, micelles, liposomes or particles can be prepared containing these lipidoids and therefore, can result in an effective delivery of the modified nucleic acids, as judged by the production of an encoded protein, following the injection of a lipidoid formulation via localized and/or systemic routes of administration. Lipidoid complexes of modified nucleic acids can be administered by various means including, but not limited to, intravenous, intramuscular, or subcutaneous routes.

In vivo delivery of nucleic acids may be affected by many parameters, including, but not limited to, the formulation composition, nature of particle PEGylation, degree of loading, oligonucleotide to lipid ratio, and biophysical parameters such as particle size (Akinc et al., Mol Ther. 2009 17:872-879; herein incorporated by reference in its entirety). As an example, small changes in the anchor chain length of poly(ethylene glycol) (PEG) lipids may result in significant effects on in vivo efficacy. Formulations with the different lipidoids, including, but not limited to penta[3-(1-laurylaminopropionyl)]-triethylenetetramine hydrochloride (TETA-5LAP; aka 98N12-5, see Murugaiah et al., Analytical Biochemistry, 401:61 (2010)), C12-200 (including derivatives and variants), and MD1, can be tested for in vivo activity.

The lipidoid referred to herein as “98N12-5” is disclosed by Akinc et al., Mol Ther. 2009 17:872-879 and is incorporated by reference in its entirety.

The lipidoid referred to herein as “C12-200” is disclosed by Love et al., Proc Natl Acad Sci USA. 2010 107:1864-1869 and Liu and Huang, Molecular Therapy. 2010 669-670; both of which are herein incorporated by reference in their entirety. The lipidoid formulations can include particles comprising either 3 or 4 or more components in addition to modified nucleic acids. As an example, formulations with certain lipidoids, include, but are not limited to, 98N12-5 and may contain 42% lipidoid, 48% cholesterol and 10% PEG (C14 alkyl chain length). As another example, formulations with certain lipidoids, include, but are not limited to, C12-200 and may contain 50% lipidoid, 10% disteroylphosphatidyl choline, 38.5% cholesterol, and 1.5% PEG-DMG.

In one embodiment, a modified nucleic acids formulated with a lipidoid for systemic intravenous administration can target the liver. For example, a final optimized intravenous formulation using modified nucleic acids, and comprising a lipid molar composition of 42% 98N12-5, 48% cholesterol, and 10% PEG-lipid with a final weight ratio of about 7.5 to 1 total lipid to modified nucleic acids, and a C14 alkyl chain length on the PEG lipid, with a mean particle size of roughly 50-60 nm, can result in the distribution of the formulation to be greater than 90% to the liver. (see, Akinc et al., Mol Ther. 2009 17:872-879; herein incorporated in its entirety). In another example, an intravenous formulation using a C12-200 (see U.S. provisional application 61/175,770 and published international application WO2010129709, each of which is herein incorporated by reference in their entirety) lipidoid may have a molar ratio of 50/10/38.5/1.5 of C12-200/disteroylphosphatidyl choline/cholesterol/PEG-DMG, with a weight ratio of 7 to 1 total lipid to modified nucleic acids, and a mean particle size of 80 nm may be effective to deliver modified nucleic acids to hepatocytes (see, Love et al., Proc Natl Acad Sci USA. 2010 107:1864-1869 herein incorporated by reference in its entirety). In another embodiment, an MD1 lipidoid-containing formulation may be used to effectively deliver modified nucleic acids to hepatocytes in vivo. The characteristics of optimized lipidoid formulations for intramuscular or subcutaneous routes may vary significantly depending on the target cell type and the ability of formulations to diffuse through the extracellular matrix into the blood stream. While a particle size of less than 150 nm may be desired for effective hepatocyte delivery due to the size of the endothelial fenestrae (see, Akinc et al., Mol Ther. 2009 17:872-879 herein incorporated by reference in its entirety), use of a lipidoid-formulated modified nucleic acids to deliver the formulation to other cells types including, but not limited to, endothelial cells, myeloid cells, and muscle cells may not be similarly size-limited. Use of lipidoid formulations to deliver siRNA in vivo to other non-hepatocyte cells such as myeloid cells and endothelium has been reported (see Akinc et al., Nat Biotechnol. 2008 26:561-569; Leuschner et al., Nat Biotechnol. 2011 29:1005-1010; Cho et al. Adv. Funct. Mater. 2009 19:3112-3118; 8^{th} International Judah Folkman Conference, Cambridge, Mass. Oct. 8-9, 2010 herein incorporated by reference in its entirety). Effective delivery to myeloid cells, such as monocytes, lipidoid formulations may have a similar component molar ratio. Different ratios of lipidoids and other components including, but not limited to, disteroylphosphatidyl choline, cholesterol and PEG-DMG, may be used to optimize the formulation of the modified nucleic acids for delivery to different cell types including, but not limited to, hepatocytes, myeloid cells, muscle cells, etc. For example, the component molar ratio may include, but is not limited to, 50% C12-200, 10% disteroylphosphatidyl choline, 38.5% cholesterol, and %1.5 PEG-DMG (see Leuschner et al., Nat Biotechnol 2011 29:1005-1010; herein incorporated by reference in its entirety). The use of lipidoid formulations for the localized delivery of nucleic acids to cells (such as, but not limited to, adipose cells and muscle cells) via either subcutaneous or intramuscular delivery, may not require all of the formulation components desired for systemic delivery, and as such may comprise only the lipidoid and the modified nucleic acids.

Combinations of different lipidoids may be used to improve the efficacy of modified nucleic acids directed protein production as the lipidoids may be able to increase cell transfection by the modified nucleic acid; and/or increase the translation of encoded protein (see Whitehead et al., Mol. Ther. 2011, 19:1688-1694, herein incorporated by reference in its entirety).

### Liposomes, Lipoplexes, and Lipid Nanoparticles

The modified nucleic acids of the invention can be formulated using one or more liposomes, lipoplexes, or lipid nanoparticles. In one embodiment, pharmaceutical compositions of modified nucleic acids include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In one embodiment, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.), In one embodiment, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; all of which are incorporated herein in their entireties.) The original manufacture method by Wheeler et al. was a detergent dialysis method, which was later improved by Jeffs et al. and is referred to as the spontaneous vesicle formation method. The liposome formulations are composed of 3 to 4 lipid components in addition to the modified nucleic acids. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In one embodiment, pharmaceutical compositions may include liposomes which may be formed to deliver modified nucleic acids which may encode at least one immunogen. The modified nucleic acids may be encapsulated by the liposome and/or it may be contained in an aqueous core which may then be encapsulated by the liposome (see International Pub. Nos. WO2012031046, WO2012031043, WO2012030901 and WO2012006378; each of which is herein incorporated by reference in their entirety). In another embodiment, the modified nucleic acids and ribonucleic acids which may encode an immunogen may be formulated in a cationic oil-in-water emulsion where the emulsion particle comprises an oil core and a cationic lipid which can interact with the modified nucleic acids anchoring the molecule to the emulsion particle (see International Pub. No. WO2012006380 herein incorporated by reference in its entirety). In yet another embodiment, the lipid formulation may include at least cationic lipid, a lipid which may enhance transfection and a least one lipid which contains a hydrophilic head group linked to a lipid moiety (International Pub. No. WO2011076807 and U.S. Pub. No. 20110200582; each of which is herein incorporated by reference in their entirety). In another embodiment, the modified nucleic acids encoding an immunogen may be formulated in a lipid vesicle which may have crosslinks between functionalized lipid bilayers (see U.S. Pub. No. 20120177724, herein incorporated by reference in its entirety).

In one embodiment, the modified nucleic acids may be formulated in a lipid vesicle which may have crosslinks between functionalized lipid bilayers.

In one embodiment, the modified nucleic acids may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine. In another embodiment, the modified nucleic acids may be formulated in a lipid-polycation complex which may further include a neutral lipid such as, but not limited to, cholesterol or dioleoyl phosphatidylethanolamine (DOPE).

The liposome formulation may be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010 28:172-176), the liposome formulation was composed of 57.1% cationic lipid, 7.1% dipalmitoylphosphatidylcholine, 34.3% cholesterol, and 1.4% PEG-c-DMA. As another example, changing the composition of the cationic lipid could more effectively deliver siRNA to various antigen presenting cells (Basha et al. Mol Ther. 2011 19:2186-2200; herein incorporated by reference in its entirety).

In some embodiments, the ratio of PEG in the LNP formulations may be increased or decreased and/or the carbon chain length of the PEG lipid may be modified from C14 to C18 to alter the pharmacokinetics and/or biodistribution of the LNP formulations. As a non-limiting example, LNP formulations may contain 1-5% of the lipid molar ratio of PEG-c-DOMG as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypolyethylene glycol) or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In one embodiment, the cationic lipid may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865 and WO2008103276, U.S. Pat. Nos. 7,893,302 and 7,404,969 and US Patent Publication No. US20100036115; each of which is herein incorporated by reference in their entirety. In another embodiment, the cationic lipid may be selected from, but not limited to, formula A described in International Publication Nos. WO2012040184, WO2011153120, WO2011149733, WO2011090965, WO2011043913, WO2011022460, WO2012061259, WO2012054365 and WO2012044638; each of which is herein incorporated by reference in their entirety. In yet another embodiment, the cationic lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115; each of which is herein incorporated by reference in their entirety. As a non-limiting example, the cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N5N˜dimethylpentacosa˜16,19-dien-8-amine, (13Z,16Z)—N,N-dimethyldocosa-13J16-dien-5-amine, (12Z,15Z)—NJN-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21Z)—N,N-dimethylheptacosa-18,21-dien-8-amine, (17Z,20Z)—N,N-dimethylhexacosa-17,20-dien-7-amine, (16Z;19Z)—N,N-dimethylpentacosa-16,19-dien-6-amine, (22Z,25Z)—N,N-dimethylhentriaconta-22,25-dien-10-amine, (21Z,24Z)—N;N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—NJN-dimethyloctacosa-19,22-dien-7-amine, N,N-dimethylheptacosan-10-amine, (20Z,23Z)—N-ethyl-N-methylnonacosa-20J23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21˜[(1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine, N,N-dimethyl-1-[(1S,2R)-2-octylcyclopropyl]hexadecan-8-amine, N,N-dimethyH-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy) methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy) methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoylo ctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N;N-dimethylnonacosa-11,20,2-trien-10-amine or a pharmaceutically acceptable salt or stereoisomer thereof.

In one embodiment, the cationic lipid may be synthesized by methods known in the art and/or as described in International Publication Nos. WO2012040184, WO2011153120, WO2011149733, WO2011090965, WO2011043913, WO2011022460, WO2012061259, WO2012054365, WO2012044638, WO2010080724 and WO201021865; each of which is herein incorporated by reference in their entirety.

In one embodiment, the LNP formulation may contain PEG-c-DOMG 3% lipid molar ratio. In another embodiment, the LNP formulation may contain PEG-c-DOMG 1.5% lipid molar ratio.

In one embodiment, the LNP formulation may contain PEG-DMG 2000 (1,2-dimyristoyl-sn-glycero-3-phosphoethanolamine-N-[methoxy(polyethylene glycol)-2000). In one embodiment, the LNP formulation may contain PEG-DMG 2000, a cationic lipid known in the art and at least one other component. In another embodiment, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see Geall et al., Nonviral delivery of self-amplifying RNA vaccines, PNAS 2012; PMID: 22908294).

In one embodiment, the LNP formulation may be formulated by the methods described in International Publication Nos. WO2011127255 or WO2008103276, each of which is herein incorporated by reference in their entirety. As a non-limiting example, modified RNA described herein may be encapsulated in LNP formulations as described in WO2011127255 and/or WO2008103276; each of which is herein incorporated by reference in their entirety.

In one embodiment, LNP formulations described herein may comprise a polycationic composition. As a non-limiting example, the polycationic composition may be selected from formula 1-60 of US Patent Publication No. US20050222064; herein incorporated by reference in its entirety. In another embodiment, the LNP formulations comprising a polycationic composition may be used for the delivery of the modified RNA described herein in vivo and/or in vitro.

In one embodiment, the LNP formulations described herein may additionally comprise a permeability enhancer molecule. Non-limiting permeability enhancer molecules are described in US Patent Publication No. US20050222064; herein incorporated by reference in its entirety.

In one embodiment, the pharmaceutical compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713)) and hyaluronan-coated liposomes (Quiet Therapeutics, Israel).

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In one embodiment, the internal ester linkage may be located on either side of the saturated carbon. Non-limiting examples of reLNPs include,

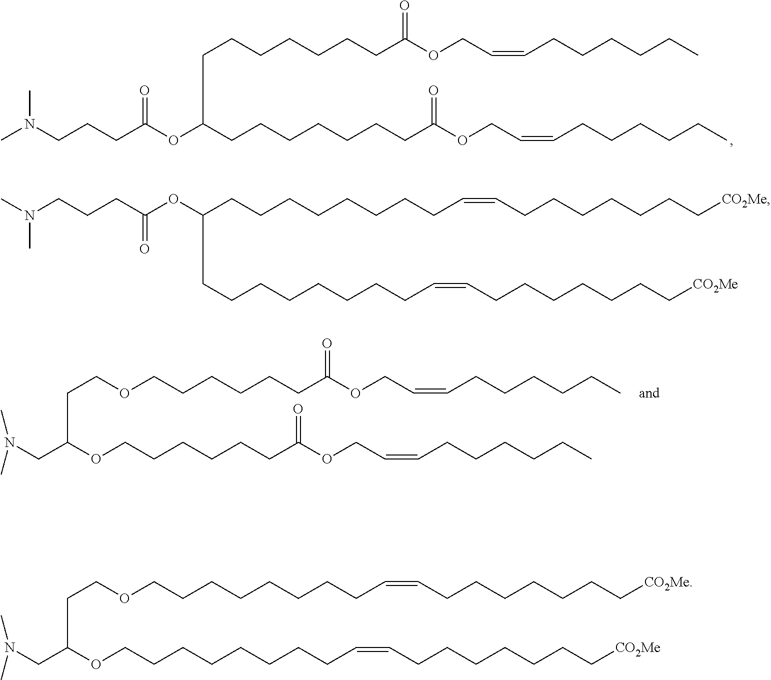

In one embodiment, an immune response may be elicited by delivering a lipid nanoparticle which may include a nanospecies, a polymer and an immunogen. (U.S. Publication No. 20120189700 and International Publication No. WO2012099805; each of which is herein incorporated by reference in their entirety). The polymer may encapsulate the nanospecies or partially encapsulate the nanospecies. The immunogen may be a recombinant protein, a modified RNA described herein. In one embodiment, the lipid nanoparticle may be formulated for use in a vaccine such as, but not limited to, against a pathogen.

Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosal tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104(5):1482-487; Lai et al. Adv Drug Deliv Rev. 2009 61(2): 158-171; each of which is herein incorporated by reference in their entirety). The transport of nanoparticles may be determined using rates of permeation and/or fluorescent microscopy techniques including, but not limited to, fluorescence recovery after photobleaching (FRAP) and high resolution multiple particle tracking (MPT).

The lipid nanoparticle engineered to penetrate mucus may comprise a polymeric material (i.e. a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material may be biodegradable and/or biocompatible. Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), and trimethylene carbonate, polyvinylpyrrolidone. The lipid nanoparticle may be coated or associated with a co-polymer such as, but not limited to, a block co-polymer, and (poly(ethylene glycol))-(poly(propylene oxide))-(poly(ethylene glycol)) triblock copolymer (see US Publication 20120121718 and US Publication 20100003337; each of which is herein incorporated by reference in their entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:2597-2600; herein incorporated by reference in its entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

The lipid nanoparticle engineered to penetrate mucus may include surface altering agents such as, but not limited to, modified nucleic acids, anionic protein (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase. The surface altering agent may be embedded or enmeshed in the particle's surface or disposed (e.g., by coating, adsorption, covalent linkage, or other process) on the surface of the lipid nanoparticle. (see US Publication 20100215580 and US Publication 20080166414; each of which is herein incorporated by reference in their entirety).

The mucus penetrating lipid nanoparticles may comprise at least one modified nucleic acids described herein. The modified nucleic acids may be encapsulated in the lipid nanoparticle and/or disposed on the surface of the particle. The modified nucleic acids may be covalently coupled to the lipid nanoparticle. Formulations of mucus penetrating lipid nanoparticles may comprise a plurality of nanoparticles. Further, the formulations may contain particles which may interact with the mucus and alter the structural and/or adhesive properties of the surrounding mucus to decrease mucoadhesion which may increase the delivery of the mucus penetrating lipid nanoparticles to the mucosal tissue.

In one embodiment, the modified nucleic acids is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, Mass.), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293 Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

In one embodiment such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133; all of which are incorporated herein by reference in its entirety). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18:1357-1364; herein incorporated by reference in its entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133; all of which are incorporated herein by reference in its entirety).

In one embodiment, the modified nucleic acids is formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see Zhang et al., ACS Nano, 2008, 2 (8), pp 1696-1702; herein incorporated by reference in its entirety).

Liposomes, lipoplexes, or lipid nanoparticles may be used to improve the efficacy of modified nucleic acids directed protein production as these formulations may be able to increase cell transfection by the modified nucleic acids; and/or increase the translation of encoded protein. One such example involves the use of lipid encapsulation to enable the effective systemic delivery of polyplex plasmid DNA (Heyes et al., Mol Ther. 2007 15:713-720; herein incorporated by reference in its entirety). The liposomes, lipoplexes, or lipid nanoparticles may also be used to increase the stability of the modified nucleic acids.

In one embodiment, the modified nucleic acids of the present invention can be formulated for controlled release and/or targeted delivery. As used herein, “controlled release” refers to a pharmaceutical composition or compound release profile that conforms to a particular pattern of release to effect a therapeutic outcome. In one embodiment, the modified nucleic acids may be encapsulated into a delivery agent described herein and/or known in the art for controlled release and/or targeted delivery. As used herein, the term “encapsulate” means to enclose, surround or encase. As it relates to the formulation of the compounds of the invention, encapsulation may be substantial, complete or partial. The term “substitantially encapsulated” means that at least greater than 50, 60, 70, 80, 85, 90, 95, 96, 97, 98, 99, 99.9, 99.9 or greater than 99.999% of the pharmaceutical composition or compound of the invention may be enclosed, surrounded or encased within the delivery agent. “Partially encapsulation” means that less than 10, 10, 20, 30, 40 50 or less of the pharmaceutical composition or compound of the invention may be enclosed, surrounded or encased within the delivery agent. Advantageously, encapsulation may be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the delivery agent.

In another embodiment, the modified nucleic acids may be encapsulated into a lipid nanoparticle or a rapidly eliminating lipid nanoparticle and the lipid nanoparticles or a rapidly eliminating lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

In one embodiment, the lipid nanoparticle may be encapsulated into any polymer or hydrogel known in the art which may form a gel when injected into a subject. As another non-limiting example, the lipid nanoparticle may be encapsulated into a polymer matrix which may be biodegradable.

In one embodiment, the modified nucleic acids formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In one embodiment, the controlled release and/or targeted delivery formulation may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In one embodiment, the modified nucleic acids of the present invention may be encapsulated in a therapeutic nanoparticle. Therapeutic nanoparticles may be formulated by methods described herein and known in the art such as, but not limited to, International Pub Nos. WO2010005740, WO2010030763, WO2010005721, WO2010005723, WO2012054923, US Pub. Nos. US20110262491, US20100104645, US20100087337, US20100068285, US20110274759, US20100068286, and U.S. Pat. No. 8,206,747; each of which is herein incorporated by reference in their entirety. In another embodiment, therapeutic polymer nanoparticles may be identified by the methods described in US Pub No. US20120140790, herein incorporated by reference in its entirety.

In one embodiment, the therapeutic nanoparticle may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the modified nucleic acids of the present invention (see International Pub No. 2010075072 and US Pub No. US20100216804 and US20110217377, each of which is herein incorporated by reference in their entirety).

In one embodiment, the therapeutic nanoparticles may be formulated to be target specific. As a non-limiting example, the therapeutic nanoparticles may include a corticosteroid (see International Pub. No. WO2011084518 the contents of which are herein incorporated by reference in its entirety). In one embodiment, the therapeutic nanoparticles may be formulated to be cancer specific. As a non-limiting example, the therapeutic nanoparticles may be formulated in nanoparticles described in International Pub No. WO2008121949, WO2010005726, WO2010005725, WO2011084521 and US Pub No. US20100069426, US20120004293 and US20100104655, each of which is herein incorporated by reference in their entirety.

In one embodiment, the nanoparticles of the present invention may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In one embodiment, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In one embodiment, the therapeutic nanoparticle comprises a diblock copolymer. As a non-limiting example the therapeutic nanoparticle comprises a PLGA-PEG block copolymer (see US Pub. No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968, herein incorporated by reference in its entirety).

In one embodiment, the therapeutic nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the therapeutic nanoparticles may comprise at least one cationic polymer described herein and/or known in the art.

In one embodiment, the therapeutic nanoparticles may comprise at least one amine-containing polymer such as, but not limited to polylysine, polyethylene imine, poly(amidoamine) dendrimers and combinations thereof.

In one embodiment, the therapeutic nanoparticles may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In another embodiment, the therapeutic nanoparticle may include a conjugation of at least one targeting ligand.

In one embodiment, the therapeutic nanoparticle may be formulated in an aqueous solution which may be used to target cancer (see International Pub No. WO2011084513 and US Pub No. US20110294717, each of which is herein incorporated by reference in their entirety).

In one embodiment, the modified nucleic acids may be encapsulated in, linked to and/or associated with synthetic nanocarriers. The synthetic nanocarriers may be formulated using methods known in the art and/or described herein. As a non-limiting example, the synthetic nanocarriers may be formulated by the methods described in International Pub Nos. WO2010005740, WO2010030763 and US Pub. Nos. US20110262491, US20100104645 and US20100087337, each of which is herein incorporated by reference in their entirety. In another embodiment, the synthetic nanocarrier formulations may be lyophilized by methods described in International Pub. No. WO2011072218 and U.S. Pat. No. 8,211,473; each of which is herein incorporated by reference in their entirety.

In one embodiment, the synthetic nanocarriers may contain reactive groups to release the modified nucleic acids described herein (see International Pub. No. WO20120952552 and US Pub No. US20120171229, each of which is herein incorporated by reference in their entirety).

In one embodiment, the synthetic nanocarriers may contain an immunostimulatory agent to enhance the immune response from delivery of the synthetic nanocarrier. As a non-limiting example, the synthetic nanocarrier may comprise a Th1 immunostimulatory agent which may enhance a Th1-based response of the immune system (see International Pub No. WO2010123569 and US Pub. No. US20110223201, each of which is herein incorporated by reference in its entirety).

In one embodiment, the synthetic nanocarriers may be formulated for targeted release. In one embodiment, the synthetic nanocarrier is formulated to release the modified nucleic acids at a specified pH and/or after a desired time interval. As a non-limiting example, the synthetic nanoparticle may be formulated to release the modified nucleic acids after 24 hours and/or at a pH of 4.5 (see International Pub. Nos. WO2010138193 and WO2010138194 and US Pub Nos. US20110020388 and US20110027217, each of which is herein incorporated by reference in their entirety).

In one embodiment, the synthetic nanocarriers may be formulated for controlled and/or sustained release of the modified nucleic acids described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety.

In one embodiment, the synthetic nanocarrier may be formulated for use as a vaccine. In one embodiment, the synthetic nanocarrier may encapsulate at least one modified nucleic acids which encodes at least one antigen. As a non-limiting example, the synthetic nanocarrier may include at least one antigen and an excipient for a vaccine dosage form (see International Pub No. WO2011150264 and US Pub No. US20110293723, each of which is herein incorporated by reference in their entirety). As another non-limiting example, a vaccine dosage form may include at least two synthetic nanocarriers with the same or different antigens and an excipient (see International Pub No. WO2011150249 and US Pub No. US20110293701, each of which is herein incorporated by reference in their entirety). The vaccine dosage form may be selected by methods described herein, known in the art and/or described in International Pub No. WO2011150258 and US Pub No. US20120027806, each of which is herein incorporated by reference in their entirety).

In one embodiment, the synthetic nanocarrier may comprise at least one modified nucleic acids which encodes at least one adjuvant. In another embodiment, the synthetic nanocarrier may comprise at least one modified nucleic acids and an adjuvant. As a non-limiting example, the synthetic nanocarrier comprising and adjuvant may be formulated by the methods described in International Pub No. WO2011150240 and US Pub No. US20110293700, each of which is herein incorporated by reference in its entirety.

In one embodiment, the synthetic nanocarrier may encapsulate at least one modified nucleic acids which encodes a peptide, fragment or region from a virus. As a non-limiting example, the synthetic nanocarrier may include, but is not limited to, the nanocarriers described in International Pub No. WO2012024621, WO201202629, WO2012024632 and US Pub No. US20120064110, US20120058153 and US20120058154, each of which is herein incorporated by reference in their entirety.

### Polymers, Biodegradable Nanoparticles, and Core-Shell Nanoparticles

The modified nucleic acids of the invention can be formulated using natural and/or synthetic polymers. Non-limiting examples of polymers which may be used for delivery include, but are not limited to, Dynamic POLYCONJUGATE™ formulations from MIRUS® Bio (Madison, Wis.) and Roche Madison (Madison, Wis.), PHASERX™ polymer formulations such as, without limitation, SMARTT POLYMER TECHNOLOGY™ (Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly(lactic-co-glycolic acid) (PLGA) polymers, RONDEL™ (RNAi/Oligonucleotide Nanoparticle Delivery) polymers (Arrowhead Research Corporation, Pasadena, Calif.) and pH responsive co-block polymers such as, but not limited to, PHASERX™ (Seattle, Wash.).

A non-limiting example of PLGA formulations include, but are not limited to, PLGA injectable depots (e.g., ELIGARD® which is formed by dissolving PLGA in 66% N-methyl-2-pyrrolidone (NMP) and the remainder being aqueous solvent and leuprolide. Once injected, the PLGA and leuprolide peptide precipitates into the subcutaneous space).

Many of these polymer approaches have demonstrated efficacy in delivering oligonucleotides in vivo into the cell cytoplasm (reviewed in deFougerolles Hum Gene Ther. 2008 19:125-132; herein incorporated by reference in its entirety). Two polymer approaches that have yielded robust in vivo delivery of nucleic acids, in this case with small interfering RNA (siRNA), are dynamic polyconjugates and cyclodextrin-based nanoparticles. The first of these delivery approaches uses dynamic polyconjugates and has been shown in vivo in mice to effectively deliver siRNA and silence endogenous target mRNA in hepatocytes (Rozema et al., Proc Natl Acad Sci USA. 2007 104:12982-12887). This particular approach is a multicomponent polymer system whose key features include a membrane-active polymer to which nucleic acid, in this case siRNA, is covalently coupled via a disulfide bond and where both PEG (for charge masking) and N-acetylgalactosamine (for hepatocyte targeting) groups are linked via pH-sensitive bonds (Rozema et al., Proc Natl Acad Sci USA. 2007 104:12982-12887). On binding to the hepatocyte and entry into the endosome, the polymer complex disassembles in the low-pH environment, with the polymer exposing its positive charge, leading to endosomal escape and cytoplasmic release of the siRNA from the polymer. Through replacement of the N-acetylgalactosamine group with a mannose group, it was shown one could alter targeting from asialoglycoprotein receptor-expressing hepatocytes to sinusoidal endothelium and Kupffer cells. Another polymer approach involves using transferrin-targeted cyclodextrin-containing polycation nanoparticles. These nanoparticles have demonstrated targeted silencing of the EWS-FLII gene product in transferrin receptor-expressing Ewing's sarcoma tumor cells (Hu-Lieskovan et al., Cancer Res. 2005 65: 8984-8982) and siRNA formulated in these nanoparticles was well tolerated in non-human primates (Heidel et al., Proc Natl Acad Sci USA 2007 104:5715-21). Both of these delivery strategies incorporate rational approaches using both targeted delivery and endosomal escape mechanisms.

The polymer formulation can permit the sustained or delayed release of modified nucleic acids (e.g., following intramuscular or subcutaneous injection). The altered release profile for the modified nucleic acids can result in, for example, translation of an encoded protein over an extended period of time. The polymer formulation may also be used to increase the stability of the modified nucleic acids. Biodegradable polymers have been previously used to protect nucleic acids other than modified nucleic acids from degradation and been shown to result in sustained release of payloads in vivo (Rozema et al., Proc Natl Acad Sci USA. 2007 104:12982-12887; Sullivan et al., Expert Opin Drug Deliv. 2010 7:1433-1446; Convertine et al., Biomacromolecules. 2010 Oct. 1; Chu et al., Acc Chem Res. 2012 Jan. 13; Manganiello et al., Biomaterials. 2012 33:2301-2309; Benoit et al., Biomacromolecules. 2011 12:2708-2714; Singha et al., Nucleic Acid Ther. 2011 2:133-147; deFougerolles Hum Gene Ther. 2008 19:125-132; Schaffert and Wagner, Gene Ther. 2008 16:1131-1138; Chaturvedi et al., Expert Opin Drug Deliv. 2011 8:1455-1468; Davis, Mol Pharm. 2009 6:659-668; Davis, Nature 2010 464:1067-1070; herein incorporated by reference in its entirety).

In one embodiment, the pharmaceutical compositions may be sustained release formulations. In a further embodiment, the sustained release formulations may be for subcutaneous delivery. Sustained release formulations may include, but are not limited to, PLGA microspheres, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

As a non-limiting example modified mRNA may be formulated in PLGA microspheres by preparing the PLGA microspheres with tunable release rates (e.g., days and weeks) and encapsulating the modified mRNA in the PLGA microspheres while maintaining the integrity of the modified mRNA during the encapsulation process. EVAc are non-biodegradeable, biocompatible polymers which are used extensively in pre-clinical sustained release implant applications (e.g., extended release products Ocusert a pilocarpine ophthalmic insert for glaucoma or progestasert a sustained release progesterone intrauterine device; transdermal delivery systems Testoderm, Duragesic and Selegiline; catheters). Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C. PEG-based surgical sealants comprise two synthetic PEG components mixed in a delivery device which can be prepared in one minute, seals in 3 minutes and is reabsorbed within 30 days. GELSITE® and natural polymers are capable of in-situ gelation at the site of administration. They have been shown to interact with protein and peptide therapeutic candidates through ionic interaction to provide a stabilizing effect.

Polymer formulations can also be selectively targeted through expression of different ligands as exemplified by, but not limited by, folate, transferrin, and N-acetylgalactosamine (GalNAc) (Benoit et al., Biomacromolecules. 2011 12:2708-2714; Rozema et al., Proc Natl Acad Sci USA. 2007 104:12982-12887; Davis, Mol Pharm. 2009 6:659-668; Davis, Nature 2010 464:1067-1070; each of which is herein incorporated by reference in its entirety).

The modified nucleic acids of the invention may be formulated with or in a polymeric compound. The polymer may include at least one polymer such as, but not limited to, polyethenes, polyethylene glycol (PEG), poly(l-lysine)(PLL), PEG grafted to PLL, cationic lipopolymer, biodegradable cationic lipopolymer, polyethyleneimine (PEI), cross-linked branched poly(alkylene imines), a polyamine derivative, a modified poloxamer, a biodegradable polymer, biodegradable block copolymer, biodegradable random copolymer, biodegradable polyester copolymer, biodegradable polyester block copolymer, biodegradable polyester block random copolymer, linear biodegradable copolymer, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers or combinations thereof.

As a non-limiting example, the modified nucleic acids of the invention may be formulated with the polymeric compound of PEG grafted with PLL as described in U.S. Pat. No. 6,177,274 herein incorporated by reference in its entirety. The formulation may be used for transfecting cells in vitro or for in vivo delivery of the modified nucleic acids. In another example, the modified nucleic acids may be suspended in a solution or medium with a cationic polymer, in a dry pharmaceutical composition or in a solution that is capable of being dried as described in U.S. Pub. Nos. 20090042829 and 20090042825 each of which are herein incorporated by reference in their entireties.

As another non-limiting example the modified nucleic acids of the invention may be formulated with a PLGA-PEG block copolymer (see US Pub. No. US20120004293 and U.S. Pat. No. 8,236,330, each of which are herein incorporated by reference in their entireties). As a non-limiting example, the modified nucleic acids of the invention may be formulated with a diblock copolymer of PEG and PLA or PEG and PLGA (see U.S. Pat. No. 8,246,968, herein incorporated by reference in its entirety).

A polyamine derivative may be used to deliver nucleic acids or to treat and/or prevent a disease or to be included in an implantable or injectable device (U.S. Pub. No. 20100260817 herein incorporated by reference in its entirety). As a non-limiting example, a pharmaceutical composition may include the modified nucleic acids and the polyamine derivative described in U.S. Pub. No. 20100260817 (the contents of which are incorporated herein by reference in its entirety).

The modified nucleic acids of the invention may be formulated with at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, modified nucleic acids of the present invention may be formulated with at least one polymer described in International Publication Nos. WO2011115862, WO2012082574 and WO2012068187, each of which are herein incorporated by reference in their entireties. In another embodiment, the modified nucleic acids of the present invention may be formulated with a polymer of formula Z as described in WO2011115862, herein incorporated by reference in its entirety. In yet another embodiment, the modified nucleic acids may be formulated with a polymer of formula Z, Z′ or Z″ as described in WO2012082574 or WO2012068187, each of which are herein incorporated by reference in their entireties. The polymers formulated with the modified RNA of the present invention may be synthesized by the methods described in WO2012082574 or WO2012068187, each of which are herein incorporated by reference in their entireties.

Formulations modified nucleic acids of the invention may include at least one amine-containing polymer such as, but not limited to polylysine, polyethylene imine, poly(amidoamine) dendrimers or combinations thereof.

For example, the modified nucleic acids of the invention may be formulated in a pharmaceutical compound including a poly(alkylene imine), a biodegradable cationic lipopolymer, a biodegradable block copolymer, a biodegradable polymer, or a biodegradable random copolymer, a biodegradable polyester block copolymer, a biodegradable polyester polymer, a biodegradable polyester random copolymer, a linear biodegradable copolymer, PAGA, a biodegradable cross-linked cationic multi-block copolymer or combinations thereof. The biodegradable cationic lipopolymer may be made by methods known in the art and/or described in U.S. Pat. No. 6,696,038, U.S. App. Nos. 20030073619 and 20040142474 each of which is herein incorporated by reference in their entireties. The poly(alkylene imine) may be made using methods known in the art and/or as described in U.S. Pub. No. 20100004315, herein incorporated by reference in its entirety. The biodegradable polymer, biodegradable block copolymer, the biodegradable random copolymer, biodegradable polyester block copolymer, biodegradable polyester polymer, or biodegradable polyester random copolymer may be made using methods known in the art and/or as described in U.S. Pat. Nos. 6,517,869 and 6,267,987, the contents of which are each incorporated herein by reference in its entirety. The linear biodegradable copolymer may be made using methods known in the art and/or as described in U.S. Pat. No. 6,652,886. The PAGA polymer may be made using methods known in the art and/or as described in U.S. Pat. No. 6,217,912 herein incorporated by reference in its entirety. The PAGA polymer may be copolymerized to form a copolymer or block copolymer with polymers such as but not limited to, poly-L-lysine, polyargine, polyornithine, histones, avidin, protamines, polylactides and poly(lactide-co-glycolides). The biodegradable cross-linked cationic multi-block copolymers may be made my methods known in the art and/or as described in U.S. Pat. No. 8,057,821 or U.S. Pub. No. 2012009145 each of which are herein incorporated by reference in their entireties. For example, the multi-block copolymers may be synthesized using linear polyethyleneimine (LPEI) blocks which have distinct patterns as compared to branched polyethyleneimines. Further, the composition or pharmaceutical composition may be made by the methods known in the art, described herein, or as described in U.S. Pub. No. 20100004315 or U.S. Pat. Nos. 6,267,987 and 6,217,912 each of which are herein incorporated by reference in their entireties.

The modified nucleic acids of the invention may be formulated with at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In one embodiment, the polymers described herein may be conjugated to a lipid-terminating PEG. As a non-limiting example, PLGA may be conjugated to a lipid-terminating PEG forming PLGA-DSPE-PEG. As another non-limiting example, PEG conjugates for use with the present invention are described in International Publication No. WO2008103276, herein incorporated by reference in its entirety.

In one embodiment, the modified RNA described herein may be conjugated with another compound. Non-limiting examples of conjugates are described in U.S. Pat. Nos. 7,964,578 and 7,833,992, each of which are herein incorporated by reference in their entireties. In another embodiment, modified RNA of the present invention may be conjugated with conjugates of formula 1-122 as described in U.S. Pat. Nos. 7,964,578 and 7,833,992, each of which are herein incorporated by reference in their entireties.

As described in U.S. Pub. No. 20100004313, herein incorporated by reference in its entirety, a gene delivery composition may include a nucleotide sequence and a poloxamer. For example, the modified nucleic acids of the present invention may be used in a gene delivery composition with the poloxamer described in U.S. Pub. No. 20100004313.

In one embodiment, the polymer formulation of the present invention may be stabilized by contacting the polymer formulation, which may include a cationic carrier, with a cationic lipopolymer which may be covalently linked to cholesterol and polyethylene glycol groups. The polymer formulation may be contacted with a cationic lipopolymer using the methods described in U.S. Pub. No. 20090042829 herein incorporated by reference in its entirety. The cationic carrier may include, but is not limited to, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B—[N—(N′,N′-Dimethylaminoethane)-carbamoyl]Cholesterol Hydrochloride (DC-Cholesterol HCl) diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride DODAC) and combinations thereof

The modified nucleic acids of the invention can also be formulated as a nanoparticle using a combination of polymers, lipids, and/or other biodegradable agents, such as, but not limited to, calcium phosphate. Components may be combined in a core-shell, hybrid, and/or layer-by-layer architecture, to allow for fine-tuning of the nanoparticle so to deliver the modified nucleic acids may be enhanced (Wang et al., Nat Mater. 2006 5:791-796; Fuller et al., Biomaterials. 2008 29:1526-1532; DeKoker et al., Adv Drug Deliv Rev. 2011 63:748-761; Endres et al., Biomaterials. 2011 32:7721-7731; Su et al., Mol Pharm. 2011 Jun. 6; 8(3):774-87; each of which is herein incorporated by reference in its entirety).

Biodegradable calcium phosphate nanoparticles in combination with lipids and/or polymers have been shown to deliver modified nucleic acids in vivo. In one embodiment, a lipid coated calcium phosphate nanoparticle, which may also contain a targeting ligand such as anisamide, may be used to deliver the modified nucleic acids of the present invention. For example, to effectively deliver siRNA in a mouse metastatic lung model a lipid coated calcium phosphate nanoparticle was used (Li et al., J Contr Rel. 2010 142: 416-421; Li et al., J Contr Rel. 2012 158:108-114; Yang et al., Mol Ther. 2012 20:609-615). This delivery system combines both a targeted nanoparticle and a component to enhance the endosomal escape, calcium phosphate, in order to improve delivery of the siRNA.

In one embodiment, calcium phosphate with a PEG-polyanion block copolymer may be used to deliver modified nucleic acids (Kazikawa et al., J Contr Rel. 2004 97:345-356; Kazikawa et al., J Contr Rel. 2006 111:368-370).

In one embodiment, a PEG-charge-conversional polymer (Pitella et al., Biomaterials. 2011 32:3106-3114) may be used to form a nanoparticle to deliver the modified nucleic acids of the present invention. The PEG-charge-conversional polymer may improve upon the PEG-polyanion block copolymers by being cleaved into a polycation at acidic pH, thus enhancing endosomal escape.

The use of core-shell nanoparticles has additionally focused on a high-throughput approach to synthesize cationic cross-linked nanogel cores and various shells (Siegwart et al., Proc Natl Acad Sci USA. 2011 108:12996-13001). The complexation, delivery, and internalization of the polymeric nanoparticles can be precisely controlled by altering the chemical composition in both the core and shell components of the nanoparticle. For example, the core-shell nanoparticles may efficiently deliver siRNA to mouse hepatocytes after they covalently attach cholesterol to the nanoparticle.

In one embodiment, a hollow lipid core comprising a middle PLGA layer and an outer neutral lipid layer containing PEG may be used to delivery of the modified nucleic acids of the present invention. As a non-limiting example, in mice bearing a luciferase-expressing tumor, it was determined that the lipid-polymer-lipid hybrid nanoparticle significantly suppressed luciferase expression, as compared to a conventional lipoplex (Shi et al, Angew Chem Int Ed. 2011 50:7027-7031).

### Peptides and Proteins

The modified nucleic acids of the invention can be formulated with peptides and/or proteins in order to increase transfection of cells by the modified nucleic acids. In one embodiment, peptides such as, but not limited to, cell penetrating peptides and proteins and peptides that enable intracellular delivery may be used to deliver pharmaceutical formulations. A non-limiting example of a cell penetrating peptide which may be used with the pharmaceutical formulations of the present invention includes a cell-penetrating peptide sequence attached to polycations that facilitates delivery to the intracellular space, e.g., HIV-derived TAT peptide, penetratins, transportans, or hCT derived cell-penetrating peptides (see, e.g., Caron et al., Mol. Ther. 3(3):310-8 (2001); Langel, Cell-Penetrating Peptides: Processes and Applications (CRC Press, Boca Raton Fla., 2002); El-Andaloussi et al., Curr. Pharm. Des. 11(28):3597-611 (2003); and Deshayes et al., Cell. Mol. Life Sci. 62(16):1839-49 (2005), all of which are incorporated herein by reference). The compositions can also be formulated to include a cell penetrating agent, e.g., liposomes, which enhance delivery of the compositions to the intracellular space. Modified nucleic acids of the invention may be complexed to peptides and/or proteins such as, but not limited to, peptides and/or proteins from Aileron Therapeutics (Cambridge, Mass.) and Permeon Biologics (Cambridge, Mass.) in order to enable intracellular delivery (Cronican et al., ACS Chem. Biol. 2010 5:747-752; McNaughton et al., Proc. Natl. Acad. Sci. USA 2009 106:6111-6116; Sawyer, Chem Biol Drug Des. 2009 73:3-6; Verdine and Hilinski, Methods Enzymol. 2012; 503:3-33; all of which are herein incorporated by reference in its entirety).

In one embodiment, the cell-penetrating polypeptide may comprise a first domain and a second domain. The first domain may comprise a supercharged polypeptide. The second domain may comprise a protein-binding partner. As used herein, “protein-binding partner” includes, but are not limited to, antibodies and functional fragments thereof, scaffold proteins, or peptides. The cell-penetrating polypeptide may further comprise an intracellular binding partner for the protein-binding partner. The cell-penetrating polypeptide may be capable of being secreted from a cell where the modified nucleic acids may be introduced.

Formulations of the including peptides or proteins may be used to increase cell transfection by the modified nucleic acids, alter the biodistribution of the modified nucleic acids (e.g., by targeting specific tissues or cell types), and/or increase the translation of encoded protein.

### Cells

The modified nucleic acids of the invention can be transfected ex vivo into cells, which are subsequently transplanted into a subject. As non-limiting examples, the pharmaceutical compositions may include red blood cells to deliver modified RNA to liver and myeloid cells, virosomes to deliver modified RNA in virus-like particles (VLPs), and electroporated cells such as, but not limited to, from MAXCYTE® (Gaithersburg, Md.) and from ERYTECH® (Lyon, France) to deliver modified RNA. Examples of use of red blood cells, viral particles and electroporated cells to deliver payloads other than modified nucleic acids have been documented (Godfrin et al., Expert Opin Biol Ther. 2012 12:127-133; Fang et al., Expert Opin Biol Ther. 2012 12:385-389; Hu et al., Proc Natl Acad Sci USA. 2011 108:10980-10985; Lund et al., Pharm Res. 2010 27:400-420; Huckriede et al., J Liposome Res. 2007; 17:39-47; Cusi, Hum Vaccin. 2006 2:1-7; de Jonge et al., Gene Ther. 2006 13:400-411; all of which are herein incorporated by reference in its entirety). The modified RNA may be delivered in synthetic VLPs synthesized by the methods described in International Pub No. WO2011085231 and US Pub No. 20110171248, each of which are herein incorporated by reference in their entireties.

Cell-based formulations of the modified nucleic acids of the invention may be used to ensure cell transfection (e.g., in the cellular carrier), alter the biodistribution of the modified nucleic acids (e.g., by targeting the cell carrier to specific tissues or cell types), and/or increase the translation of encoded protein.

Introduction into Cells

A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion.

The technique of sonoporation, or cellular sonication, is the use of sound (e.g., ultrasonic frequencies) for modifying the permeability of the cell plasma membrane. Sonoporation methods are known to those in the art and are taught for example as it relates to bacteria in US Patent Publication 20100196983 and as it relates to other cell types in, for example, US Patent Publication 20100009424, each of which are incorporated herein by reference in their entirety.

Electroporation techniques are also well known in the art. In one embodiment, modified nucleic acids may be delivered by electroporation as described in Example 8.

### Hyaluronidase

The intramuscular or subcutaneous localized injection of modified nucleic acids of the invention can include hyaluronidase, which catalyzes the hydrolysis of hyaluronan. By catalyzing the hydrolysis of hyaluronan, a constituent of the interstitial barrier, hyaluronidase lowers the viscosity of hyaluronan, thereby increasing tissue permeability (Frost, Expert Opin. Drug Deliv. (2007) 4:427-440; herein incorporated by reference in its entirety). It is useful to speed their dispersion and systemic distribution of encoded proteins produced by transfected cells. Alternatively, the hyaluronidase can be used to increase the number of cells exposed to a modified nucleic acids of the invention administered intramuscularly or subcutaneously.

### Nanoparticle Mimics

The modified nucleic acids of the invention may be encapsulated within and/or absorbed to a nanoparticle mimic. A nanoparticle mimic can mimic the delivery function organisms or particles such as, but not limited to, pathogens, viruses, bacteria, fungus, parasites, prions and cells. As a non-limiting example the modified nucleic acids of the invention may be encapsulated in a non-viron particle which can mimic the delivery function of a virus (see International Pub. No. WO2012006376 herein incorporated by reference in its entirety).

### Nanotubes

The modified nucleic acids of the invention can be attached or otherwise bound to at least one nanotube such as, but not limited to, rosette nanotubes, rosette nanotubes having twin bases with a linker, carbon nanotubes and/or single-walled carbon nanotubes, The modified nucleic acids may be bound to the nanotubes through forces such as, but not limited to, steric, ionic, covalent and/or other forces.

In one embodiment, the nanotube can release one or more modified nucleic acids into cells. The size and/or the surface structure of at least one nanotube may be altered so as to govern the interaction of the nanotubes within the body and/or to attach or bind to the modified nucleic acids disclosed herein. In one embodiment, the building block and/or the functional groups attached to the building block of the at least one nanotube may be altered to adjust the dimensions and/or properties of the nanotube. As a non-limiting example, the length of the nanotubes may be altered to hinder the nanotubes from passing through the holes in the walls of normal blood vessels but still small enough to pass through the larger holes in the blood vessels of tumor tissue.

In one embodiment, at least one nanotube may also be coated with delivery enhancing compounds including polymers, such as, but not limited to, polyethylene glycol. In another embodiment, at least one nanotube and/or the modified mRNA may be mixed with pharmaceutically acceptable excipients and/or delivery vehicles.

In one embodiment, the modified mRNA are attached and/or otherwise bound to at least one rosette nanotube. The rosette nanotubes may be formed by a process known in the art and/or by the process described in International Publication No. WO2012094304, herein incorporated by reference in its entirety. At least one modified mRNA may be attached and/or otherwise bound to at least one rosette nanotube by a process as described in International Publication No. WO2012094304, herein incorporated by reference in its entirety, where rosette nanotubes or modules forming rosette nanotubes are mixed in aqueous media with at least one modified mRNA under conditions which may cause at least one modified mRNA to attach or otherwise bind to the rosette nanotubes.

### Conjugates

The modified nucleic acids of the invention include conjugates, such as a modified nucleic acids covalently linked to a carrier or targeting group, or including two encoding regions that together produce a fusion protein (e.g., bearing a targeting group and therapeutic protein or peptide).

The conjugates of the invention include a naturally occurring substance, such as a protein (e.g., human serum albumin (HSA), low-density lipoprotein (LDL), high-density lipoprotein (HDL), or globulin); an carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g. an aptamer). Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Representative U.S. patents that teach the preparation of polynucleotide conjugates, particularly to RNA, include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; each of which is herein incorporated by reference in their entireties.

In one embodiment, the conjugate of the present invention may function as a carrier for the modified nucleic acids of the present invention. The conjugate may comprise a cationic polymer such as, but not limited to, polyamine, polylysine, polyalkylenimine, and polyethylenimine which may be grafted to with poly(ethylene glycol). As a non-limiting example, the conjugate may be similar to the polymeric conjugate and the method of synthesizing the polymeric conjugate described in U.S. Pat. No. 6,586,524 herein incorporated by reference in its entirety.

The conjugates can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Targeting groups can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Targeting groups may also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, multivalent fucose, or aptamers. The ligand can be, for example, a lipopolysaccharide, or an activator of p38 MAP kinase.

The targeting group can be any ligand that is capable of targeting a specific receptor. Examples include, without limitation, folate, GalNAc, galactose, mannose, mannose-6P, apatamers, integrin receptor ligands, chemokine receptor ligands, transferrin, biotin, serotonin receptor ligands, PSMA, endothelin, GCPII, somatostatin, LDL, and HDL ligands. In particular embodiments, the targeting group is an aptamer. The aptamer can be unmodified or have any combination of modifications disclosed herein.

In one embodiment, pharmaceutical compositions of the present invention may include chemical modifications such as, but not limited to, modifications similar to locked nucleic acids.

Representative U.S. patents that teach the preparation of locked nucleic acid (LNA) such as those from Santaris, include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found, for example, in Nielsen et al., Science, 1991, 254, 1497-1500.

Some embodiments featured in the invention include modified nucleic acids with phosphorothioate backbones and oligonucleosides with other modified backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— [wherein the native phosphodiester backbone is represented as —O—P(O)₂—O—CH₂—] of the above-referenced U.S. Pat. No. 5,489,677, and the amide backbones of the above-referenced U.S. Pat. No. 5,602,240. In some embodiments, the polynucleotides featured herein have morpholino backbone structures of the above-referenced U.S. Pat. No. 5,034,506.

Modifications at the 2′ position may also aid in delivery. Preferably, modifications at the 2′ position are not located in a polypeptide-coding sequence, i.e., not in a translatable region. Modifications at the 2′ position may be located in a 5′UTR, a 3′UTR and/or a tailing region. Modifications at the 2′ position can include one of the following at the 2′ position: H (i.e., 2′-deoxy); F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)_{.n}OCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, the modified nucleic acids include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties, or a group for improving the pharmacodynamic properties, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below. Other modifications include 2′-methoxy (2′-OCH₃), 2′-aminopropoxy (2′-OCH₂CH₂CH₂NH₂) and 2′-fluoro (2′-F). Similar modifications may also be made at other positions, particularly the 3′ position of the sugar on the 3′ terminal nucleotide or in 2′-5′ linked dsRNAs and the 5′ position of 5′ terminal nucleotide. Polynucleotides of the invention may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920 and each of which is herein incorporated by reference.

In still other embodiments, the modified nucleic acids is covalently conjugated to a cell penetrating polypeptide. The cell-penetrating peptide may also include a signal sequence. The conjugates of the invention can be designed to have increased stability; increased cell transfection; and/or altered the biodistribution (e.g., targeted to specific tissues or cell types).

### Self-Assembled Nucleic Acid Nanoparticles

Self-assembled nanoparticles have a well-defined size which may be precisely controlled as the nucleic acid strands may be easily reprogrammable. For example, the optimal particle size for a cancer-targeting nanodelivery carrier is 20-100 nm as a diameter greater than 20 nm avoids renal clearance and enhances delivery to certain tumors through enhanced permeability and retention effect. Using self-assembled nucleic acid nanoparticles a single uniform population in size and shape having a precisely controlled spatial orientation and density of cancer-targeting ligands for enhanced delivery. As a non-limiting example, oligonucleotide nanoparticles were prepared using programmable self-assembly of short DNA fragments and therapeutic siRNAs. These nanoparticles are molecularly identical with controllable particle size and target ligand location and density. The DNA fragments and siRNAs self-assembled into a one-step reaction to generate DNA/siRNA tetrahedral nanoparticles for targeted in vivo delivery. (Lee et al., Nature Nanotechnology 2012 7:389-393).

### Excipients

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this present disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

### Delivery

The present disclosure encompasses the delivery of modified nucleic acids encoding proteins or complexes, and/or pharmaceutical, prophylactic, diagnostic, or imaging compositions thereof, by any appropriate route taking into consideration likely advances in the sciences of drug delivery. Delivery may be naked or formulated.

In general the most appropriate route of administration will depend upon a variety of factors including the nature of the modified nucleic acids encoding proteins or complexes comprising modified nucleic acids encoding proteins associated with at least one agent to be delivered (e.g., its stability in the environment of the gastrointestinal tract, bloodstream, etc.), the condition of the patient (e.g., whether the patient is able to tolerate particular routes of administration), etc. The present disclosure encompasses the delivery of the pharmaceutical, prophylactic, diagnostic, or imaging compositions by any appropriate route taking into consideration likely advances in the sciences of drug delivery.

### Naked Delivery

The modified nucleic acids of the present invention may be delivered to a cell naked. As used herein in, “naked” refers to delivering modified nucleic acids from agents which promote transfection. For example, the modified nucleic acids delivered to the cell may contain no modifications. The naked modified nucleic acids may be delivered to the cell using routes of administration known in the art and described herein.

### Formulated Delivery

The modified nucleic acids of the present invention may be formulated, using the methods described herein. The formulations may contain modified nucleic acids which may be modified and/or unmodified. The formulations may further include, but are not limited to, cell penetration agents, a pharmaceutically acceptable carrier, a delivery agent, a bioerodible or biocompatible polymer, a solvent, and a sustained-release delivery depot. The formulated modified nucleic acids may be delivered to the cell using routes of administration known in the art and described herein.

The compositions may also be formulated for direct delivery to an organ or tissue in any of several ways in the art including, but not limited to, direct soaking or bathing, via a catheter, by gels, powder, ointments, creams, gels, lotions, and/or drops, by using substrates such as fabric or biodegradable materials coated or impregnated with the compositions, and the like.

### Administration

The modified nucleic acids of the present invention may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops.

In one embodiment, provided are compositions for generation of an in vivo depot containing a modified nucleic acid. For example, the composition contains a bioerodible, biocompatible polymer, a solvent present in an amount effective to plasticize the polymer and form a gel therewith, and an engineered ribonucleic acid. In certain embodiments the composition also includes a cell penetration agent as described herein. In other embodiments, the composition also contains a thixotropic amount of a thixotropic agent mixable with the polymer so as to be effective to form a thixotropic composition. Further compositions include a stabilizing agent, a bulking agent, a chelating agent, or a buffering agent.

In other embodiments, provided are sustained-release delivery depots, such as for administration of a modified nucleic acid an environment (meaning an organ or tissue site) in a patient. Such depots generally contain a modified nucleic acid and a flexible chain polymer where both the modified nucleic acid and the flexible chain polymer are entrapped within a porous matrix of a crosslinked matrix protein. Usually, the pore size is less than 1 mm, such as 900 nm, 800 nm, 700 nm, 600 nm, 500 nm, 400 nm, 300 nm, 200 nm, 100 nm, or less than 100 nm. Usually the flexible chain polymer is hydrophilic. Usually the flexible chain polymer has a molecular weight of at least 50 kDa, such as 75 kDa, 100 kDa, 150 kDa, 200 kDa, 250 kDa, 300 kDa, 400 kDa, 500 kDa, or greater than 500 kDa. Usually the flexible chain polymer has a persistence length of less than 10%, such as 9, 8, 7, 6, 5, 4, 3, 2, 1 or less than 1% of the persistence length of the matrix protein. Usually the flexible chain polymer has a charge similar to that of the matrix protein. In some embodiments, the flexible chain polymer alters the effective pore size of a matrix of crosslinked matrix protein to a size capable of sustaining the diffusion of the modified nucleic acid from the matrix into a surrounding tissue comprising a cell into which the modified nucleic acid is capable of entering.

In specific embodiments, compositions may be administered in a way which allows them cross the blood-brain barrier, vascular barrier, or other epithelial barrier. Non-limiting routes of administration for the modified nucleic acids of the present invention are described below.

The present disclosure provides methods comprising administering modified nucleic acids, proteins or complexes in accordance with the present disclosure to a subject in need thereof. Modified nucleic acids, proteins or complexes, or pharmaceutical, imaging, diagnostic, or prophylactic compositions thereof, may be administered to a subject using any amount and any route of administration effective for preventing, treating, diagnosing, or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like. Compositions in accordance with the present disclosure are typically formulated in dosage unit form for ease of administration and uniformity of dosage. It will be understood, however, that the total daily usage of the compositions of the present disclosure will be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Modified nucleic acids, proteins to be delivered and/or pharmaceutical, prophylactic, diagnostic, or imaging compositions thereof may be administered to animals, such as mammals (e.g., humans, domesticated animals, cats, dogs, mice, rats, etc.). In some embodiments, pharmaceutical, prophylactic, diagnostic, or imaging compositions thereof are administered to humans.

Modified nucleic acids, proteins to be delivered and/or pharmaceutical, prophylactic, diagnostic, or imaging compositions thereof in accordance with the present disclosure may be administered by any route. In some embodiments, proteins and/or pharmaceutical, prophylactic, diagnostic, or imaging compositions thereof, are administered by one or more of a variety of routes, including oral, intravenous, intramuscular, intra-arterial, intramedullary, intrathecal, subcutaneous, intraventricular, transdermal, interdermal, rectal, intravaginal, intraperitoneal, topical (e.g. by powders, ointments, creams, gels, lotions, and/or drops), mucosal, nasal, buccal, enteral, vitreal, intratumoral, sublingual; by intratracheal instillation, bronchial instillation, and/or inhalation; as an oral spray, nasal spray, and/or aerosol, and/or through a portal vein catheter. In some embodiments, proteins or complexes, and/or pharmaceutical, prophylactic, diagnostic, or imaging compositions thereof, are administered by systemic intravenous injection. In specific embodiments, proteins or complexes and/or pharmaceutical, prophylactic, diagnostic, or imaging compositions thereof may be administered intravenously and/or orally. In specific embodiments, proteins or complexes, and/or pharmaceutical, prophylactic, diagnostic, or imaging compositions thereof, may be administered in a way which allows the modified nucleic acid, protein or complex to cross the blood-brain barrier, vascular barrier, or other epithelial barrier.

### Parenteral and Injectible Administration

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Rectal and Vaginal Administration

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### Oral Administration

Liquid dosage forms for oral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

### Topical or Transdermal Administration

As described herein, compositions containing the modified nucleic acids of the invention may be formulated for administration topically. The skin may be an ideal target site for delivery as it is readily accessible. Gene expression may be restricted not only to the skin, potentially avoiding nonspecific toxicity, but also to specific layers and cell types within the skin.

The site of cutaneous expression of the delivered compositions will depend on the route of nucleic acid delivery. Three routes are commonly considered to deliver modified nucleic acids to the skin: (i) topical application (e.g. for local/regional treatment); (ii) intradermal injection (e.g. for local/regional treatment); and (iii) systemic delivery (e.g. for treatment of dermatologic diseases that affect both cutaneous and extracutaneous regions). Modified nucleic acids can be delivered to the skin by several different approaches known in the art. Most topical delivery approaches have been shown to work for delivery of DNA, such as but not limited to, topical application of non-cationic liposome-DNA complex, cationic liposome-DNA complex, particle-mediated (gene gun), puncture-mediated gene transfections, and viral delivery approaches. After delivery of the nucleic acid, gene products have been detected in a number of different skin cell types, including, but not limited to, basal keratinocytes, sebaceous gland cells, dermal fibroblasts and dermal macrophages.

In one embodiment, the invention provides for a variety of dressings (e.g., wound dressings) or bandages (e.g., adhesive bandages) for conveniently and/or effectively carrying out methods of the present invention. Typically dressing or bandages may comprise sufficient amounts of pharmaceutical compositions and/or modified nucleic acids described herein to allow a user to perform multiple treatments of a subject(s).

In one embodiment, the invention provides for the modified nucleic acids compositions to be delivered in more than one injection.

In one embodiment, before topical and/or transdermal administration at least one area of tissue, such as skin, may be subjected to a device and/or solution which may increase permeability. In one embodiment, the tissue may be subjected to an abrasion device to increase the permeability of the skin (see U.S. Patent Publication No. 20080275468, herein incorporated by reference in its entirety). In another embodiment, the tissue may be subjected to an ultrasound enhancement device. An ultrasound enhancement device may include, but is not limited to, the devices described in U.S. Publication No. 20040236268 and U.S. Pat. Nos. 6,491,657 and 6,234,990; each of which are herein incorporated by reference in their entireties. Methods of enhancing the permeability of tissue are described in U.S. Publication Nos. 20040171980 and 20040236268 and U.S. Pat. No. 6,190,315; each of which are herein incorporated by reference in their entireties.

In one embodiment, a device may be used to increase permeability of tissue before delivering formulations of modified mRNA described herein. The permeability of skin may be measured by methods known in the art and/or described in U.S. Pat. No. 6,190,315, herein incorporated by reference in its entirety. As a non-limiting example, a modified mRNA formulation may be delivered by the drug delivery methods described in U.S. Pat. No. 6,190,315, herein incorporated by reference in its entirety.

In another non-limiting example tissue may be treated with a eutectic mixture of local anesthetics (EMLA) cream before, during and/or after the tissue may be subjected to a device which may increase permeability. Katz et al. (Anesth Analg (2004); 98:371-76; herein incorporated by reference in its entirety) showed that using the EMLA cream in combination with a low energy, an onset of superficial cutaneous analgesia was seen as fast as 5 minutes after a pretreatment with a low energy ultrasound.

In one embodiment, enhancers may be applied to the tissue before, during, and/or after the tissue has been treated to increase permeability. Enhancers include, but are not limited to, transport enhancers, physical enhancers, and cavitation enhancers. Non-limiting examples of enhancers are described in U.S. Pat. No. 6,190,315, herein incorporated by reference in its entirety.

In one embodiment, a device may be used to increase permeability of tissue before delivering formulations of modified mRNA described herein, which may further contain a substance that invokes an immune response. In another non-limiting example, a formulation containing a substance to invoke an immune response may be delivered by the methods described in U.S. Publication Nos. 20040171980 and 20040236268; each of which are herein incorporated by reference in their entireties.

Dosage forms for topical and/or transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

### Depot Administration

As described herein, in some embodiments, the composition is formulated in depots for extended release. Generally, a specific organ or tissue (a “target tissue”) is targeted for administration.

In some aspects of the invention, the nucleic acids (particularly ribonucleic acids encoding polypeptides) are spatially retained within or proximal to a target tissue. Provided are method of providing a composition to a target tissue of a mammalian subject by contacting the target tissue (which contains one or more target cells) with the composition under conditions such that the composition, in particular the nucleic acid component(s) of the composition, is substantially retained in the target tissue, meaning that at least 10, 20, 30, 40, 50, 60, 70, 80, 85, 90, 95, 96, 97, 98, 99, 99.9, 99.99 or greater than 99.99% of the composition is retained in the target tissue. Advantageously, retention is determined by measuring the amount of the nucleic acid present in the composition that enters one or more target cells. For example, at least 1, 5, 10, 20, 30, 40, 50, 60, 70, 80, 85, 90, 95, 96, 97, 98, 99, 99.9, 99.99 or greater than 99.99% of the nucleic acids administered to the subject are present intracellularly at a period of time following administration. For example, intramuscular injection to a mammalian subject is performed using an aqueous composition containing a ribonucleic acid and a transfection reagent, and retention of the composition is determined by measuring the amount of the ribonucleic acid present in the muscle cells.

Aspects of the invention are directed to methods of providing a composition to a target tissue of a mammalian subject, by contacting the target tissue (containing one or more target cells) with the composition under conditions such that the composition is substantially retained in the target tissue. a ribonucleic acid engineered to avoid an innate immune response of a cell into which the ribonucleic acid enters, where the ribonucleic acid contains a nucleotide sequence encoding a polypeptide of interest, under conditions such that the polypeptide of interest is produced in at least one target cell. The compositions generally contain a cell penetration agent, although “naked” nucleic acid (such as nucleic acids without a cell penetration agent or other agent) is also contemplated, and a pharmaceutically acceptable carrier.

In some circumstances, the amount of a protein produced by cells in a tissue is desirably increased. Preferably, this increase in protein production is spatially restricted to cells within the target tissue. Thus, provided are methods of increasing production of a protein of interest in a tissue of a mammalian subject. A composition is provided that contains a ribonucleic acid that is engineered to avoid an innate immune response of a cell into which the ribonucleic acid enters and encodes the polypeptide of interest and the composition is characterized in that a unit quantity of composition has been determined to produce the polypeptide of interest in a substantial percentage of cells contained within a predetermined volume of the target tissue.

In some embodiments, the composition includes a plurality of different ribonucleic acids, where one or more than one of the ribonucleic acids is engineered to avoid an innate immune response of a cell into which the ribonucleic acid enters, and where one or more than one of the ribonucleic acids encodes a polypeptide of interest. Optionally, the composition also contains a cell penetration agent to assist in the intracellular delivery of the ribonucleic acid. A determination is made of the dose of the composition required to produce the polypeptide of interest in a substantial percentage of cells contained within the predetermined volume of the target tissue (generally, without inducing significant production of the polypeptide of interest in tissue adjacent to the predetermined volume, or distally to the target tissue). Subsequent to this determination, the determined dose is introduced directly into the tissue of the mammalian subject.

In one embodiment, the invention provides for the modified nucleic acids to be delivered in more than one injection or by split dose injections.

In one embodiment, the invention may be retained near target tissue using a small disposable drug reservoir or patch pump. Non-limiting examples of patch pumps include those manufactured and/or sold by BD®, (Franklin Lakes, N.J.), Insulet Corporation (Bedford, Mass.), SteadyMed Therapeutics (San Francisco, Calif.), Medtronic (Minneapolis, Minn.), UniLife (York, Pa.), Valeritas (Bridgewater, N.J.), and SpringLeaf Therapeutics (Boston, Mass.).

### Pulmonary Administration

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and active ingredient may constitute 0.1% to 20% (w/w) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

### Intranasal, Nasal and Buccal Administration

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

### Ophthalmic Administration

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other opthalmically-administrable formulations which are useful include those which comprise the active ingredient in microcrystalline form and/or in a liposomal preparation. Ear drops and/or eye drops are contemplated as being within the scope of this present disclosure.

### Payload Administration: Detectable Agents and Therapeutic Agents

The modified nucleic acids described herein can be used in a number of different scenarios in which delivery of a substance (the “payload”) to a biological target is desired, for example delivery of detectable substances for detection of the target, or delivery of a therapeutic agent. Detection methods can include, but are not limited to, both imaging in vitro and in vivo imaging methods, e.g., immunohistochemistry, bioluminescence imaging (BLI), Magnetic Resonance Imaging (MM), positron emission tomography (PET), electron microscopy, X-ray computed tomography, Raman imaging, optical coherence tomography, absorption imaging, thermal imaging, fluorescence reflectance imaging, fluorescence microscopy, fluorescence molecular tomographic imaging, nuclear magnetic resonance imaging, X-ray imaging, ultrasound imaging, photoacoustic imaging, lab assays, or in any situation where tagging/staining/imaging is required.

The modified nucleic acids can be designed to include both a linker and a payload in any useful orientation. For example, a linker having two ends is used to attach one end to the payload and the other end to the nucleobase, such as at the C-7 or C-8 positions of the deaza-adenosine or deaza-guanosine or to the N-3 or C-5 positions of cytosine or uracil. The polynucleotide of the invention can include more than one payload (e.g., a label and a transcription inhibitor), as well as a cleavable linker.

In one embodiment, the modified nucleotide is a modified 7-deaza-adenosine triphosphate, where one end of a cleavable linker is attached to the C7 position of 7-deaza-adenine, the other end of the linker is attached to an inhibitor (e.g., to the C5 position of the nucleobase on a cytidine), and a label (e.g., Cy5) is attached to the center of the linker (see, e.g., compound 1 of A*pCp C5 Parg Capless in FIG. 5 and columns 9 and 10 of U.S. Pat. No. 7,994,304, incorporated herein by reference). Upon incorporation of the modified 7-deaza-adenosine triphosphate to an encoding region, the resulting polynucleotide having a cleavable linker attached to a label and an inhibitor (e.g., a polymerase inhibitor). Upon cleavage of the linker (e.g., with reductive conditions to reduce a linker having a cleavable disulfide moiety), the label and inhibitor are released. Additional linkers and payloads (e.g., therapeutic agents, detectable labels, and cell penetrating payloads) are described herein.

For example, the modified nucleic acids described herein can be used in reprogramming induced pluripotent stem cells (iPS cells), which can directly track cells that are transfected compared to total cells in the cluster. In another example, a drug that may be attached to the modified nucleic acids via a linker and may be fluorescently labeled can be used to track the drug in vivo, e.g. intracellularly. Other examples include, but are not limited to, the use of modified nucleic acids in reversible drug delivery into cells.

The modified nucleic acids described herein can be used in intracellular targeting of a payload, e.g., detectable or therapeutic agent, to specific organelle. Exemplary intracellular targets can include, but are not limited to, the nuclear localization for advanced mRNA processing, or a nuclear localization sequence (NLS) linked to the mRNA containing an inhibitor.

In addition, the modified nucleic acids described herein can be used to deliver therapeutic agents to cells or tissues, e.g., in living animals. For example, the modified nucleic acids described herein can be used to deliver highly polar chemotherapeutics agents to kill cancer cells. The modified nucleic acids attached to the therapeutic agent through a linker can facilitate member permeation allowing the therapeutic agent to travel into a cell to reach an intracellular target.

In another example, the modified nucleic acids can be attached to the modified nucleic acids a viral inhibitory peptide (VIP) through a cleavable linker. The cleavable linker can release the VIP and dye into the cell. In another example, the modified nucleic acids can be attached through the linker to an ADP-ribosylate, which is responsible for the actions of some bacterial toxins, such as cholera toxin, diphtheria toxin, and pertussis toxin. These toxin proteins are ADP-ribosyltransferases that modify target proteins in human cells. For example, cholera toxin ADP-ribosylates G proteins modifies human cells by causing massive fluid secretion from the lining of the small intestine, which results in life-threatening diarrhea.

In some embodiments, the payload may be a therapeutic agent such as a cytotoxin, radioactive ion, chemotherapeutic, or other therapeutic agent. A cytotoxin or cytotoxic agent includes any agent that may be detrimental to cells. Examples include, but are not limited to, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020 incorporated herein in its entirety), rachelmycin (CC-1065, see U.S. Pat. Nos. 5,475,092, 5,585,499, and 5,846,545, all of which are incorporated herein by reference), and analogs or homologs thereof. Radioactive ions include, but are not limited to iodine (e.g., iodine 125 or iodine 131), strontium 89, phosphorous, palladium, cesium, iridium, phosphate, cobalt, yttrium 90, samarium 153, and praseodymium. Other therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, rachelmycin (CC-1065), melphalan, carmustine (BSNU), lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In some embodiments, the payload may be a detectable agent, such as various organic small molecules, inorganic compounds, nanoparticles, enzymes or enzyme substrates, fluorescent materials, luminescent materials (e.g., luminol), bioluminescent materials (e.g., luciferase, luciferin, and aequorin), chemiluminescent materials, radioactive materials (e.g., ¹⁸F, ⁶⁷Ga, ^{81m}Kr, ⁸²Rb, ¹¹¹In, ¹²³I, ¹³³Xe, ²⁰¹Tl, ¹²⁵I, ³⁵S, ¹⁴C, ³H, or ^{99m}Tc (e.g., as pertechnetate (technetate(VII), TcO₄⁻)), and contrast agents (e.g., gold (e.g., gold nanoparticles), gadolinium (e.g., chelated Gd), iron oxides (e.g., superparamagnetic iron oxide (SPIO), monocrystalline iron oxide nanoparticles (MIONs), and ultrasmall superparamagnetic iron oxide (USPIO)), manganese chelates (e.g., Mn-DPDP), barium sulfate, iodinated contrast media (iohexol), microbubbles, or perfluorocarbons). Such optically-detectable labels include for example, without limitation, 4-acetamido-4′-isothiocyanatostilbene-2,2′disulfonic acid; acridine and derivatives (e.g., acridine and acridine isothiocyanate); 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives (e.g., coumarin, 7-amino-4-methylcoumarin (AMC, Coumarin 120), and 7-amino-4-trifluoromethylcoumarin (Coumarin 151)); cyanine dyes; cyanosine; 4′,6-diaminidino-2-phenylindole (DAPI); 5′ 5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]-naphthalene-1-sulfonyl chloride (DNS, dansylchloride); 4-dimethylaminophenylazophenyl-4′-isothiocyanate (DABITC); eosin and derivatives (e.g., eosin and eosin isothiocyanate); erythrosin and derivatives (e.g., erythrosin B and erythrosin isothiocyanate); ethidium; fluorescein and derivatives (e.g., 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, X-rhodamine-5-(and-6)-isothiocyanate (QFITC or XRITC), and fluorescamine); 2-[2-[3-[[1,3-dihydro-1,1-dimethyl-3-(3-sulfopropyl)-2H-benz[e]indol-2-ylidene]ethylidene]-2-[4-(ethoxycarbonyl)-1-piperazinyl]-1-cyclopenten-1-yl]ethenyl]-1,1-dimethyl-3-(3-sulforpropyl)-1H-benz[e]indolium hydroxide, inner salt, compound with n,n-diethylethanamine(1:1) (IR144); 5-chloro-2-[2-[3-[(5-chloro-3-ethyl-2(3H)-benzothiazol-ylidene)ethylidene]-2-(diphenylamino)-1-cyclopenten-1-yl]ethenyl]-3-ethyl benzothiazolium perchlorate (IR140); Malachite Green isothiocyanate; 4-methylumbelliferone orthocresolphthalein; nitrotyrosine; pararosaniline; Phenol Red; B-phycoerythrin; o-phthaldialdehyde; pyrene and derivatives (e.g., pyrene, pyrene butyrate, and succinimidyl 1-pyrene); butyrate quantum dots; Reactive Red 4 (Cibacron™ Brilliant Red 3B-A); rhodamine and derivatives (e.g., 6-carboxy-X-rhodamine (ROX), 6-carboxyrhodamine (R6G), lissamine rhodamine B sulfonyl chloride rhodarnine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′tetramethyl-6-carboxyrhodamine (TAMRA) tetramethyl rhodamine, and tetramethyl rhodamine isothiocyanate (TRITC)); riboflavin; rosolic acid; terbium chelate derivatives; Cyanine-3 (Cy3); Cyanine-5 (Cy5); cyanine-5.5 (Cy5.5), Cyanine-7 (Cy7); IRD 700; IRD 800; Alexa 647; La Jolta Blue; phthalo cyanine; and naphthalo cyanine.

In some embodiments, the detectable agent may be a non-detectable pre-cursor that becomes detectable upon activation (e.g., fluorogenic tetrazine-fluorophore constructs (e.g., tetrazine-BODIPY FL, tetrazine-Oregon Green 488, or tetrazine-BODIPY TMR-X) or enzyme activatable fluorogenic agents (e.g., PROSENSE® (VisEn Medical))). In vitro assays in which the enzyme labeled compositions can be used include, but are not limited to, enzyme linked immunosorbent assays (ELISAs), immunoprecipitation assays, immunofluorescence, enzyme immunoassays (EIA), radioimmunoassays (RIA), and Western blot analysis. Combination

Modified nucleic acids encoding proteins or complexes may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that improve their bioavailability, reduce and/or modify their metabolism, inhibit their excretion, and/or modify their distribution within the body.

In some embodiments, the present disclosure encompasses the delivery of pharmaceutical, prophylactic, diagnostic, or imaging compositions in combination with agents that may improve their bioavailability, reduce and/or modify their metabolism, inhibit their excretion, and/or modify their distribution within the body. As a non-limiting example, the modified nucleic acids may be used in combination with a pharmaceutical agent for the treatment of cancer or to control hyperproliferative cells. In U.S. Pat. No. 7,964,571, herein incorporated by reference in its entirety, a combination therapy for the treatment of solid primary or metastasized tumor is described using a pharmaceutical composition including a DNA plasmid encoding for interleukin-12 with a lipopolymer and also administering at least one anticancer agent or chemotherapeutic. Further, the modified nucleic acids of the present invention that encodes anti-proliferative molecules may be in a pharmaceutical composition with a lipopolymer (see e.g., U.S. Pub. No. 20110218231, herein incorporated by reference in its entirety, claiming a pharmaceutical composition comprising a DNA plasmid encoding an anti-proliferative molecule and a lipopolymer) which may be administered with at least one chemotherapeutic or anticancer agent.

It will further be appreciated that therapeutically, prophylactically, diagnostically, or imaging active agents utilized in combination may be administered together in a single composition or administered separately in different compositions. In general, it is expected that agents utilized in combination with be utilized at levels that do not exceed the levels at which they are utilized individually. In some embodiments, the levels utilized in combination will be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer in accordance with the present disclosure may be administered concurrently with a chemotherapeutic agent), or they may achieve different effects (e.g., control of any adverse effects).

### Cell Penetrating Payload

In some embodiments, the modified nucleotides and modified nucleic acid molecules, which are incorporated into a nucleic acid, e.g., RNA or mRNA, can also include a payload that can be a cell penetrating moiety or agent that enhances intracellular delivery of the compositions. For example, the compositions can include, but are not limited to, a cell-penetrating peptide sequence that facilitates delivery to the intracellular space, e.g., HIV-derived TAT peptide, penetratins, transportans, or hCT derived cell-penetrating peptides, see, e.g., Caron et al., (2001) Mol Ther. 3(3):310-8; Langel, Cell-Penetrating Peptides: Processes and Applications (CRC Press, Boca Raton Fla. 2002); El-Andaloussi et al., (2005) Curr Pharm Des. 11(28):3597-611; and Deshayes et al., (2005) Cell Mol Life Sci. 62(16):1839-49; all of which are incorporated herein by reference. The compositions can also be formulated to include a cell penetrating agent, e.g., liposomes, which enhance delivery of the compositions to the intracellular space

### Biological Target

The modified nucleotides and modified nucleic acid molecules described herein, which are incorporated into a nucleic acid, e.g., RNA or mRNA, can be used to deliver a payload to any biological target for which a specific ligand exists or can be generated. The ligand can bind to the biological target either covalently or non-covalently.

Examples of biological targets include, but are not limited to, biopolymers, e.g., antibodies, nucleic acids such as RNA and DNA, proteins, enzymes; examples of proteins include, but are not limited to, enzymes, receptors, and ion channels. In some embodiments the target may be a tissue- or a cell-type specific marker, e.g., a protein that is expressed specifically on a selected tissue or cell type. In some embodiments, the target may be a receptor, such as, but not limited to, plasma membrane receptors and nuclear receptors; more specific examples include, but are not limited to, G-protein-coupled receptors, cell pore proteins, transporter proteins, surface-expressed antibodies, HLA proteins, MHC proteins and growth factor receptors.

### Dosing

The present invention provides methods comprising administering modified mRNAs and their encoded proteins or complexes in accordance with the invention to a subject in need thereof. Nucleic acids, proteins or complexes, or pharmaceutical, imaging, diagnostic, or prophylactic compositions thereof, may be administered to a subject using any amount and any route of administration effective for preventing, treating, diagnosing, or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, compositions in accordance with the present disclosure may be administered at dosage levels sufficient to deliver from about 0.0001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

According to the present invention, it has been discovered that administration of modified nucleic acids in split-dose regimens produce higher levels of proteins in mammalian subjects. As used herein, a “split dose” is the division of single unit dose or total daily dose into two or more doses, e.g, two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In one embodiment, the modified nucleic acids of the present invention are administered to a subject in split doses. The modified nucleic acids may be formulated in buffer only or in a formulation described herein.

### Dosage Forms

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, subcutaneous).

### Liquid Dosage Forms

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions may be mixed with solubilizing agents such as CREMOPHOR®, alcohols, oils, modified oils, glycols, polysorbates, cyclodextrins, polymers, and/or combinations thereof.

### Injectable

Injectable preparations, for example, sterile injectable aqueous or oleaginous suspensions may be formulated according to the known art and may include suitable dispersing agents, wetting agents, and/or suspending agents. Sterile injectable preparations may be sterile injectable solutions, suspensions, and/or emulsions in nontoxic parenterally acceptable diluents and/or solvents, for example, a solution in 1,3-butanediol. Among the acceptable vehicles and solvents that may be employed include, but are not limited to, water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it may be desirable to slow the absorption of the active ingredient from subcutaneous or intramuscular injection. This may be accomplished by the use of a liquid suspension of crystalline or amorphous material with poor water solubility. The rate of absorption of modified mRNA then depends upon its rate of dissolution which, in turn, may depend upon crystal size and crystalline form. Alternatively, delayed absorption of a parenterally administered modified mRNA may be accomplished by dissolving or suspending the modified mRNA in an oil vehicle. Injectable depot forms are made by forming microencapsule matrices of the modified mRNA in biodegradable polymers such as polylactide-polyglycolide. Depending upon the ratio of modified mRNA to polymer and the nature of the particular polymer employed, the rate of modified mRNA release can be controlled. Examples of other biodegradable polymers include, but are not limited to, poly(orthoesters) and poly(anhydrides). Depot injectable formulations may be prepared by entrapping the modified mRNA in liposomes or microemulsions which are compatible with body tissues.

### Pulmonary

Formulations described herein as being useful for pulmonary delivery may also be used for intranasal delivery of a pharmaceutical composition. Another formulation suitable for intranasal administration may be a coarse powder comprising the active ingredient and having an average particle from about 0.2 μm to 500 μm. Such a formulation may be administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, contain about 0.1% to 20% (w/w) active ingredient, where the balance may comprise an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

### Coatings or Shells

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### Kits

The present disclosure provides a variety of kits for conveniently and/or effectively carrying out methods of the present disclosure. Typically kits will comprise sufficient amounts and/or numbers of components to allow a user to perform multiple treatments of a subject(s) and/or to perform multiple experiments. In one aspect, the present invention provides kits for protein production, comprising a first modified nucleic acids comprising a translatable region. The kit may further comprise packaging and instructions and/or a delivery agent to form a formulation composition. The delivery agent may comprise a saline, a buffered solution, a lipidoid or any delivery agent disclosed herein.

In one embodiment, the buffer solution may include sodium chloride, calcium chloride, phosphate and/or EDTA. In another embodiment, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium. In a further embodiment, the buffer solutions may be precipitated or it may be lyophilized. The amount of each component may be varied to enable consistent, reproducible higher concentration saline or simple buffer formulations. The components may also be varied in order to increase the stability of modified RNA in the buffer solution over a period of time and/or under a variety of conditions.

In one aspect, the disclosure provides kits for protein production, comprising a first isolated nucleic acid comprising a translatable region and a nucleic acid modification, wherein the nucleic acid is capable of evading an innate immune response of a cell into which the first isolated nucleic acid is introduced, and packaging and instructions.

In one aspect, the disclosure provides kits for protein production, comprising: a first isolated nucleic acid comprising a translatable region, provided in an amount effective to produce a desired amount of a protein encoded by the translatable region when introduced into a target cell; a second nucleic acid comprising an inhibitory nucleic acid, provided in an amount effective to substantially inhibit the innate immune response of the cell; and packaging and instructions.

In one aspect, the disclosure provides kits for protein production, comprising a first isolated nucleic acid comprising a translatable region and a nucleoside modification, wherein the nucleic acid exhibits reduced degradation by a cellular nuclease, and packaging and instructions.

In one aspect, the disclosure provides kits for protein production, comprising a first isolated nucleic acid comprising a translatable region and at least one nucleoside modification, wherein the nucleic acid exhibits reduced degradation by a cellular nuclease; a second nucleic acid comprising an inhibitory nucleic acid; and packaging and instructions.

### Devices

The present invention provides for devices which may incorporate modified nucleic acids that encode polypeptides of interest. These devices contain in a stable formulation the reagents to synthesize a nucleic acid in a formulation available to be immediately delivered to a subject in need thereof, such as a human patient. Non-limiting examples of such a polypeptide of interest include a growth factor and/or angiogenesis stimulator for wound healing, a peptide antibiotic to facilitate infection control, and an antigen to rapidly stimulate an immune response to a newly identified virus.

In some embodiments the device is self-contained, and is optionally capable of wireless remote access to obtain instructions for synthesis and/or analysis of the generated modified nucleic acids. The device is capable of mobile synthesis of at least one modified nucleic acids and preferably an unlimited number of different modified nucleic acids. In certain embodiments, the device is capable of being transported by one or a small number of individuals. In other embodiments, the device is scaled to fit on a benchtop or desk. In other embodiments, the device is scaled to fit into a suitcase, backpack or similarly sized object. In another embodiment, the device may be a point of care or handheld device. In further embodiments, the device is scaled to fit into a vehicle, such as a car, truck or ambulance, or a military vehicle such as a tank or personnel carrier. The information necessary to generate a ribonucleic acid encoding polypeptide of interest is present within a computer readable medium present in the device.

In one embodiment, a device may be used to assess levels of a protein which has been administered in the form of a modified nucleic acids. The device may comprise a blood, urine or other biofluidic test.

In some embodiments, the device is capable of communication (e.g., wireless communication) with a database of nucleic acid and polypeptide sequences. The device contains at least one sample block for insertion of one or more sample vessels. Such sample vessels are capable of accepting in liquid or other form any number of materials such as template DNA, nucleotides, enzymes, buffers, and other reagents. The sample vessels are also capable of being heated and cooled by contact with the sample block. The sample block is generally in communication with a device base with one or more electronic control units for the at least one sample block. The sample block preferably contains a heating module, such heating molecule capable of heating and/or cooling the sample vessels and contents thereof to temperatures between about −20 C and above +100 C. The device base is in communication with a voltage supply such as a battery or external voltage supply. The device also contains means for storing and distributing the materials for RNA synthesis.

Optionally, the sample block contains a module for separating the synthesized nucleic acids. Alternatively, the device contains a separation module operably linked to the sample block. Preferably the device contains a means for analysis of the synthesized nucleic acid. Such analysis includes sequence identity (demonstrated such as by hybridization), absence of non-desired sequences, measurement of integrity of synthesized mRNA (such has by microfluidic viscometry combined with spectrophotometry), and concentration and/or potency of modified nucleic acids (such as by spectrophotometry).

In certain embodiments, the device is combined with a means for detection of pathogens present in a biological material obtained from a subject, e.g., the IBIS PLEX-ID system (Abbott, Abbott Park, Ill.) for microbial identification.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662; each of which is herein incorporated by reference in its entirety. Intradermal compositions may be administered by devices which limit the effective penetration length of a needle into the skin, such as those described in PCT publication WO 99/34850 (the contents of which are herein incorporated by reference in its entirety) and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537; herein incorporated by reference in its entirety. Ballistic powder/particle delivery devices which use compressed gas to accelerate vaccine in powder form through the outer layers of the skin to the dermis are suitable.

Alternatively or additionally, conventional syringes may be used in the classical mantoux method of intradermal administration.

In some embodiments, the device may be a pump or comprise a catheter for administration of compounds or compositions of the invention across the blood brain barrier. Such devices include but are not limited to a pressurized olfactory delivery device, iontophoresis devices, multi-layered microfluidic devices, and the like. Such devices may be portable or stationary. They may be implantable or externally tethered to the body or combinations thereof.

Devices for administration may be employed to deliver the modified nucleic acids of the present invention according to single, multi- or split-dosing regimens taught herein. Such devices are described below.

Method and devices known in the art for multi-administration to cells, organs and tissues are contemplated for use in conjunction with the methods and compositions disclosed herein as embodiments of the present invention. These include, for example, those methods and devices having multiple needles, hybrid devices employing for example lumens or catheters as well as devices utilizing heat, electric current or radiation driven mechanisms.

According to the present invention, these multi-administration devices may be utilized to deliver the single, multi- or split doses contemplated herein.

A method for delivering therapeutic agents to a solid tissue has been described by Bahrami et al. and is taught for example in US Patent Publication 20110230839, the contents of which are incorporated herein by reference in their entirety. According to Bahrami, an array of needles is incorporated into a device which delivers a substantially equal amount of fluid at any location in said solid tissue along each needle's length.

A device for delivery of biological material across the biological tissue has been described by Kodgule et al. and is taught for example in US Patent Publication 20110172610, the contents of which are incorporated herein by reference in their entirety. According to Kodgule, multiple hollow micro-needles made of one or more metals and having outer diameters from about 200 microns to about 350 microns and lengths of at least 100 microns are incorporated into the device which delivers peptides, proteins, carbohydrates, nucleic acid molecules, lipids and other pharmaceutically active ingredients or combinations thereof.

A delivery probe for delivering a therapeutic agent to a tissue has been described by Gunday et al. and is taught for example in US Patent Publication 20110270184, the contents of which are incorporated herein by reference in their entirety. According to Gunday, multiple needles are incorporated into the device which moves the attached capsules between an activated position and an inactivated position to force the agent out of the capsules through the needles.

A multiple-injection medical apparatus has been described by Assaf and is taught for example in US Patent Publication 20110218497, the contents of which are incorporated herein by reference in their entirety. According to Assaf, multiple needles are incorporated into the device which has a chamber connected to one or more of said needles and a means for continuously refilling the chamber with the medical fluid after each injection.

In one embodiment, the modified nucleic acids are administered subcutaneously or intramuscularly via at least 3 needles to three different, optionally adjacent, sites simultaneously, or within a 60 minutes period (e.g., administration to 4, 5, 6, 7, 8, 9, or 10 sites simultaneously or within a 60 minute period). The split doses can be administered simultaneously to adjacent tissue using the devices described in U.S. Patent Publication Nos. 20110230839 and 20110218497, each of which is incorporated herein by reference in their entirety.

An at least partially implantable system for injecting a substance into a patient's body, in particular a penis erection stimulation system has been described by Forsell and is taught for example in US Patent Publication 20110196198, the contents of which are incorporated herein by reference in their entirety. According to Forsell, multiple needles are incorporated into the device which is implanted along with one or more housings adjacent the patient's left and right corpora cavernosa. A reservoir and a pump are also implanted to supply drugs through the needles.

A method for the transdermal delivery of a therapeutic effective amount of iron has been described by Berenson and is taught for example in US Patent Publication 20100130910, the contents of which are incorporated herein by reference in their entirety. According to Berenson, multiple needles may be used to create multiple micro channels in stratum corneum to enhance transdermal delivery of the ionic iron on an iontophoretic patch.

A method for delivery of biological material across the biological tissue has been described by Kodgule et al and is taught for example in US Patent Publication 20110196308, the contents of which are incorporated herein by reference in their entirety. According to Kodgule, multiple biodegradable microneedles containing a therapeutic active ingredient are incorporated in a device which delivers proteins, carbohydrates, nucleic acid molecules, lipids and other pharmaceutically active ingredients or combinations thereof.

A transdermal patch comprising a botulinum toxin composition has been described by Donovan and is taught for example in US Patent Publication 20080220020, the contents of which are incorporated herein by reference in their entirety. According to Donovan, multiple needles are incorporated into the patch which delivers botulinum toxin under stratum corneum through said needles which project through the stratum corneum of the skin without rupturing a blood vessel.

A small, disposable drug reservoir, or patch pump, which can hold approximately 0.2 to 15 mL of liquid formulations can be placed on the skin and deliver the formulation continuously subcutaneously using a small bore needed (e.g., 26 to 34 gauge). As non-limiting examples, the patch pump may be 50 mm by 76 mm by 20 mm spring loaded having a 30 to 34 gauge needle (BD™ Microinfuser, Franklin Lakes N.J.), 41 mm by 62 mm by 17 mm with a 2 mL reservoir used for drug delivery such as insulin (OMNIPOD®, Insulet Corporation Bedford, Mass.), or 43-60 mm diameter, 10 mm thick with a 0.5 to 10 mL reservoir (PATCHPUMP®, SteadyMed Therapeutics, San Francisco, Calif.). Further, the patch pump may be battery powered and/or rechargeable.

A cryoprobe for administration of an active agent to a location of cryogenic treatment has been described by Toubia and is taught for example in US Patent Publication 20080140061, the contents of which are incorporated herein by reference in their entirety. According to Toubia, multiple needles are incorporated into the probe which receives the active agent into a chamber and administers the agent to the tissue.

A method for treating or preventing inflammation or promoting healthy joints has been described by Stock et al and is taught for example in US Patent Publication 20090155186, the contents of which are incorporated herein by reference in their entirety. According to Stock, multiple needles are incorporated in a device which administers compositions containing signal transduction modulator compounds.

A multi-site injection system has been described by Kimmell et al. and is taught for example in US Patent Publication 20100256594, the contents of which are incorporated herein by reference in their entirety. According to Kimmell, multiple needles are incorporated into a device which delivers a medication into a stratum corneum through the needles.

A method for delivering interferons to the intradermal compartment has been described by Dekker et al. and is taught for example in US Patent Publication 20050181033, the contents of which are incorporated herein by reference in their entirety. According to Dekker, multiple needles having an outlet with an exposed height between 0 and 1 mm are incorporated into a device which improves pharmacokinetics and bioavailability by delivering the substance at a depth between 0.3 mm and 2 mm.

A method for delivering genes, enzymes and biological agents to tissue cells has described by Desai and is taught for example in US Patent Publication 20030073908, the contents of which are incorporated herein by reference in their entirety. According to Desai, multiple needles are incorporated into a device which is inserted into a body and delivers a medication fluid through said needles.

A method for treating cardiac arrhythmias with fibroblast cells has been described by Lee et al and is taught for example in US Patent Publication 20040005295, the contents of which are incorporated herein by reference in their entirety. According to Lee, multiple needles are incorporated into the device which delivers fibroblast cells into the local region of the tissue.

A method using a magnetically controlled pump for treating a brain tumor has been described by Shachar et al. and is taught for example in U.S. Pat. No. 7,799,012 (method) and U.S. Pat. No. 7,799,016 (device), the contents of which are incorporated herein by reference in their entirety. According Shachar, multiple needles were incorporated into the pump which pushes a medicating agent through the needles at a controlled rate.

Methods of treating functional disorders of the bladder in mammalian females have been described by Versi et al. and are taught for example in U.S. Pat. No. 8,029,496, the contents of which are incorporated herein by reference in their entirety. According to Versi, an array of micro-needles is incorporated into a device which delivers a therapeutic agent through the needles directly into the trigone of the bladder.

A micro-needle transdermal transport device has been described by Angel et al and is taught for example in U.S. Pat. No. 7,364,568, the contents of which are incorporated herein by reference in their entirety. According to Angel, multiple needles are incorporated into the device which transports a substance into a body surface through the needles which are inserted into the surface from different directions. The micro-needle transdermal transport device may be a solid micro-needle system or a hollow micro-needle system. As a non-limiting example, the solid micro-needle system may have up to a 0.5 mg capacity, with 300-1500 solid micro-needles per cm² about 150-700 μm tall coated with a drug. The micro-needles penetrate the stratum corneum and remain in the skin for short duration (e.g., 20 seconds to 15 minutes). In another example, the hollow micro-needle system has up to a 3 mL capacity to deliver liquid formulations using 15-20 microneedles per cm2 being approximately 950 μm tall. The micro-needles penetrate the skin to allow the liquid formulations to flow from the device into the skin. The hollow micro-needle system may be worn from 1 to 30 minutes depending on the formulation volume and viscosity.

A device for subcutaneous infusion has been described by Dalton et al and is taught for example in U.S. Pat. No. 7,150,726, the contents of which are incorporated herein by reference in their entirety. According to Dalton, multiple needles are incorporated into the device which delivers fluid through the needles into a subcutaneous tissue.

A device and a method for intradermal delivery of vaccines and gene therapeutic agents through microcannula have been described by Mikszta et al. and are taught for example in U.S. Pat. No. 7,473,247, the contents of which are incorporated herein by reference in their entirety. According to Mitszta, at least one hollow micro-needle is incorporated into the device which delivers the vaccines to the subject's skin to a depth of between 0.025 mm and 2 mm.

A method of delivering insulin has been described by Pettis et al and is taught for example in U.S. Pat. No. 7,722,595, the contents of which are incorporated herein by reference in their entirety. According to Pettis, two needles are incorporated into a device wherein both needles insert essentially simultaneously into the skin with the first at a depth of less than 2.5 mm to deliver insulin to intradermal compartment and the second at a depth of greater than 2.5 mm and less than 5.0 mm to deliver insulin to subcutaneous compartment.

Cutaneous injection delivery under suction has been described by Kochamba et al. and is taught for example in U.S. Pat. No. 6,896,666, the contents of which are incorporated herein by reference in their entirety. According to Kochamba, multiple needles in relative adjacency with each other are incorporated into a device which injects a fluid below the cutaneous layer.

A device for withdrawing or delivering a substance through the skin has been described by Down et al and is taught for example in U.S. Pat. No. 6,607,513, the contents of which are incorporated herein by reference in their entirety. According to Down, multiple skin penetrating members which are incorporated into the device have lengths of about 100 microns to about 2000 microns and are about 30 to 50 gauge.

A device for delivering a substance to the skin has been described by Palmer et al and is taught for example in U.S. Pat. No. 6,537,242, the contents of which are incorporated herein by reference in their entirety. According to Palmer, an array of micro-needles is incorporated into the device which uses a stretching assembly to enhance the contact of the needles with the skin and provides a more uniform delivery of the substance.

A perfusion device for localized drug delivery has been described by Zamoyski and is taught for example in U.S. Pat. No. 6,468,247, the contents of which are incorporated herein by reference in their entirety. According to Zamoyski, multiple hypodermic needles are incorporated into the device which injects the contents of the hypodermics into a tissue as said hypodermics are being retracted.

A method for enhanced transport of drugs and biological molecules across tissue by improving the interaction between micro-needles and human skin has been described by Prausnitz et al. and is taught for example in U.S. Pat. No. 6,743,211, the contents of which are incorporated herein by reference in their entirety. According to Prausnitz, multiple micro-needles are incorporated into a device which is able to present a more rigid and less deformable surface to which the micro-needles are applied.

A device for intraorgan administration of medicinal agents has been described by Ting et al and is taught for example in U.S. Pat. No. 6,077,251, the contents of which are incorporated herein by reference in their entirety. According to Ting, multiple needles having side openings for enhanced administration are incorporated into a device which by extending and retracting said needles from and into the needle chamber forces a medicinal agent from a reservoir into said needles and injects said medicinal agent into a target organ.

A multiple needle holder and a subcutaneous multiple channel infusion port has been described by Brown and is taught for example in U.S. Pat. No. 4,695,273, the contents of which are incorporated herein by reference in their entirety. According to Brown, multiple needles on the needle holder are inserted through the septum of the infusion port and communicate with isolated chambers in said infusion port.

A dual hypodermic syringe has been described by Horn and is taught for example in U.S. Pat. No. 3,552,394, the contents of which are incorporated herein by reference in their entirety. According to Horn, two needles incorporated into the device are spaced apart less than 68 mm and may be of different styles and lengths, thus enabling injections to be made to different depths.

A syringe with multiple needles and multiple fluid compartments has been described by Hershberg and is taught for example in U.S. Pat. No. 3,572,336, the contents of which are incorporated herein by reference in their entirety. According to Hershberg, multiple needles are incorporated into the syringe which has multiple fluid compartments and is capable of simultaneously administering incompatible drugs which are not able to be mixed for one injection.

A surgical instrument for intradermal injection of fluids has been described by Eliscu et al. and is taught for example in U.S. Pat. No. 2,588,623, the contents of which are incorporated herein by reference in their entirety. According to Eliscu, multiple needles are incorporated into the instrument which injects fluids intradermally with a wider disperse.

An apparatus for simultaneous delivery of a substance to multiple breast milk ducts has been described by Hung and is taught for example in EP 1818017, the contents of which are incorporated herein by reference in their entirety. According to Hung, multiple lumens are incorporated into the device which inserts though the orifices of the ductal networks and delivers a fluid to the ductal networks.

A catheter for introduction of medications to the tissue of a heart or other organs has been described by Tkebuchava and is taught for example in WO2006138109, the contents of which are incorporated herein by reference in their entirety. According to Tkebuchava, two curved needles are incorporated which enter the organ wall in a flattened trajectory.

Devices for delivering medical agents have been described by Mckay et al. and are taught for example in WO2006118804, the content of which are incorporated herein by reference in their entirety. According to Mckay, multiple needles with multiple orifices on each needle are incorporated into the devices to facilitate regional delivery to a tissue, such as the interior disc space of a spinal disc.

A method for directly delivering an immunomodulatory substance into an intradermal space within a mammalian skin has been described by Pettis and is taught for example in WO2004020014, the contents of which are incorporated herein by reference in their entirety. According to Pettis, multiple needles are incorporated into a device which delivers the substance through the needles to a depth between 0.3 mm and 2 mm.

Methods and devices for administration of substances into at least two compartments in skin for systemic absorption and improved pharmacokinetics have been described by Pettis et al. and are taught for example in WO2003094995, the contents of which are incorporated herein by reference in their entirety. According to Pettis, multiple needles having lengths between about 300 μm and about 5 mm are incorporated into a device which delivers to intradermal and subcutaneous tissue compartments simultaneously.

A drug delivery device with needles and a roller has been described by Zimmerman et al. and is taught for example in WO2012006259, the contents of which are incorporated herein by reference in their entirety. According to Zimmerman, multiple hollow needles positioned in a roller are incorporated into the device which delivers the content in a reservoir through the needles as the roller rotates.

Methods and Devices Utilizing Catheters and/or Lumens

Methods and devices using catheters and lumens may be employed to administer the modified nucleic acids of the present invention on a single, multi- or split dosing schedule. Such methods and devices are described below.

A catheter-based delivery of skeletal myoblasts to the myocardium of damaged hearts has been described by Jacoby et al and is taught for example in US Patent Publication 20060263338, the contents of which are incorporated herein by reference in their entirety. According to Jacoby, multiple needles are incorporated into the device at least part of which is inserted into a blood vessel and delivers the cell composition through the needles into the localized region of the subject's heart.

An apparatus for treating asthma using neurotoxin has been described by Deem et al and is taught for example in US Patent Publication 20060225742, the contents of which are incorporated herein by reference in their entirety. According to Deem, multiple needles are incorporated into the device which delivers neurotoxin through the needles into the bronchial tissue.

A method for administering multiple-component therapies has been described by Nayak and is taught for example in U.S. Pat. No. 7,699,803, the contents of which are incorporated herein by reference in their entirety. According to Nayak, multiple injection cannulas may be incorporated into a device wherein depth slots may be included for controlling the depth at which the therapeutic substance is delivered within the tissue.

A surgical device for ablating a channel and delivering at least one therapeutic agent into a desired region of the tissue has been described by McIntyre et al and is taught for example in U.S. Pat. No. 8,012,096, the contents of which are incorporated herein by reference in their entirety. According to McIntyre, multiple needles are incorporated into the device which dispenses a therapeutic agent into a region of tissue surrounding the channel and is particularly well suited for transmyocardial revascularization operations.

Methods of treating functional disorders of the bladder in mammalian females have been described by Versi et al and are taught for example in U.S. Pat. No. 8,029,496, the contents of which are incorporated herein by reference in their entirety. According to Versi, an array of micro-needles is incorporated into a device which delivers a therapeutic agent through the needles directly into the trigone of the bladder.

A device and a method for delivering fluid into a flexible biological barrier have been described by Yeshurun et al. and are taught for example in U.S. Pat. No. 7,998,119 (device) and U.S. Pat. No. 8,007,466 (method), the contents of which are incorporated herein by reference in their entirety. According to Yeshurun, the micro-needles on the device penetrate and extend into the flexible biological barrier and fluid is injected through the bore of the hollow micro-needles.

A method for epicardially injecting a substance into an area of tissue of a heart having an epicardial surface and disposed within a torso has been described by Bonner et al and is taught for example in U.S. Pat. No. 7,628,780, the contents of which are incorporated herein by reference in their entirety. According to Bonner, the devices have elongate shafts and distal injection heads for driving needles into tissue and injecting medical agents into the tissue through the needles.

A device for sealing a puncture has been described by Nielsen et al and is taught for example in U.S. Pat. No. 7,972,358, the contents of which are incorporated herein by reference in their entirety. According to Nielsen, multiple needles are incorporated into the device which delivers a closure agent into the tissue surrounding the puncture tract.

A method for myogenesis and angiogenesis has been described by Chiu et al. and is taught for example in U.S. Pat. No. 6,551,338, the contents of which are incorporated herein by reference in their entirety. According to Chiu, 5 to 15 needles having a maximum diameter of at least 1.25 mm and a length effective to provide a puncture depth of 6 to 20 mm are incorporated into a device which inserts into proximity with a myocardium and supplies an exogeneous angiogenic or myogenic factor to said myocardium through the conduits which are in at least some of said needles.

A method for the treatment of prostate tissue has been described by Bolmsj et al. and is taught for example in U.S. Pat. No. 6,524,270, the contents of which are incorporated herein by reference in their entirety. According to Bolmsj, a device comprising a catheter which is inserted through the urethra has at least one hollow tip extendible into the surrounding prostate tissue. An astringent and analgesic medicine is administered through said tip into said prostate tissue.

A method for infusing fluids to an intraosseous site has been described by Findlay et al. and is taught for example in U.S. Pat. No. 6,761,726, the contents of which are incorporated herein by reference in their entirety. According to Findlay, multiple needles are incorporated into a device which is capable of penetrating a hard shell of material covered by a layer of soft material and delivers a fluid at a predetermined distance below said hard shell of material.

A device for injecting medications into a vessel wall has been described by Vigil et al. and is taught for example in U.S. Pat. No. 5,713,863, the contents of which are incorporated herein by reference in their entirety. According to Vigil, multiple injectors are mounted on each of the flexible tubes in the device which introduces a medication fluid through a multi-lumen catheter, into said flexible tubes and out of said injectors for infusion into the vessel wall.

A catheter for delivering therapeutic and/or diagnostic agents to the tissue surrounding a bodily passageway has been described by Faxon et al. and is taught for example in U.S. Pat. No. 5,464,395, the contents of which are incorporated herein by reference in their entirety. According to Faxon, at least one needle cannula is incorporated into the catheter which delivers the desired agents to the tissue through said needles which project outboard of the catheter.

Balloon catheters for delivering therapeutic agents have been described by Orr and are taught for example in WO2010024871, the contents of which are incorporated herein by reference in their entirety. According to Orr, multiple needles are incorporated into the devices which deliver the therapeutic agents to different depths within the tissue.

### Methods and Devices Utilizing Electrical Current

Methods and devices utilizing electric current may be employed to deliver the modified nucleic acids of the present invention according to the single, multi- or split dosing regimens taught herein. Such methods and devices are described below.

An electro collagen induction therapy device has been described by Marquez and is taught for example in US Patent Publication 20090137945, the contents of which are incorporated herein by reference in their entirety. According to Marquez, multiple needles are incorporated into the device which repeatedly pierce the skin and draw in the skin a portion of the substance which is applied to the skin first.

An electrokinetic system has been described by Etheredge et al. and is taught for example in US Patent Publication 20070185432, the contents of which are incorporated herein by reference in their entirety. According to Etheredge, micro-needles are incorporated into a device which drives by an electrical current the medication through the needles into the targeted treatment site.

An iontophoresis device has been described by Matsumura et al. and is taught for example in U.S. Pat. No. 7,437,189, the contents of which are incorporated herein by reference in their entirety. According to Matsumura, multiple needles are incorporated into the device which is capable of delivering ionizable drug into a living body at higher speed or with higher efficiency.

Intradermal delivery of biologically active agents by needle-free injection and electroporation has been described by Hoffmann et al and is taught for example in U.S. Pat. No. 7,171,264, the contents of which are incorporated herein by reference in their entirety. According to Hoffmann, one or more needle-free injectors are incorporated into an electroporation device and the combination of needle-free injection and electroporation is sufficient to introduce the agent into cells in skin, muscle or mucosa.

A method for electropermeabilization-mediated intracellular delivery has been described by Lundkvist et al. and is taught for example in U.S. Pat. No. 6,625,486, the contents of which are incorporated herein by reference in their entirety. According to Lundkvist, a pair of needle electrodes is incorporated into a catheter. Said catheter is positioned into a body lumen followed by extending said needle electrodes to penetrate into the tissue surrounding said lumen. Then the device introduces an agent through at least one of said needle electrodes and applies electric field by said pair of needle electrodes to allow said agent pass through the cell membranes into the cells at the treatment site.

A delivery system for transdermal immunization has been described by Levin et al. and is taught for example in WO2006003659, the contents of which are incorporated herein by reference in their entirety. According to Levin, multiple electrodes are incorporated into the device which applies electrical energy between the electrodes to generate micro channels in the skin to facilitate transdermal delivery.

A method for delivering RF energy into skin has been described by Schomacker and is taught for example in WO2011163264, the contents of which are incorporated herein by reference in their entirety. According to Schomacker, multiple needles are incorporated into a device which applies vacuum to draw skin into contact with a plate so that needles insert into skin through the holes on the plate and deliver RF energy.

In one aspect, the disclosure provides kits for protein production, comprising a first isolated nucleic acid comprising a translatable region and a nucleic acid modification, wherein the nucleic acid is capable of evading an innate immune response of a cell into which the first isolated nucleic acid is introduced, and packaging and instructions.

In one aspect, the disclosure provides kits for protein production, comprising: a first isolated nucleic acid comprising a translatable region, provided in an amount effective to produce a desired amount of a protein encoded by the translatable region when introduced into a target cell; a second nucleic acid comprising an inhibitory nucleic acid, provided in an amount effective to substantially inhibit the innate immune response of the cell; and packaging and instructions.

In one aspect, the disclosure provides kits for protein production, comprising a first isolated nucleic acid comprising a translatable region and a nucleoside modification, wherein the nucleic acid exhibits reduced degradation by a cellular nuclease, and packaging and instructions.

In one aspect, the disclosure provides kits for protein production, comprising a first isolated nucleic acid comprising a translatable region and at least two different nucleoside modifications, wherein the nucleic acid exhibits reduced degradation by a cellular nuclease, and packaging and instructions.

In one aspect, the disclosure provides kits for protein production, comprising a first isolated nucleic acid comprising a translatable region and at least one nucleoside modification, wherein the nucleic acid exhibits reduced degradation by a cellular nuclease; a second nucleic acid comprising an inhibitory nucleic acid; and packaging and instructions.

In some embodiments, the first isolated nucleic acid comprises messenger RNA (mRNA). In some embodiments the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In some embodiments, the mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In another aspect, the disclosure provides compositions for protein production, comprising a first isolated nucleic acid comprising a translatable region and a nucleoside modification, wherein the nucleic acid exhibits reduced degradation by a cellular nuclease, and a mammalian cell suitable for translation of the translatable region of the first nucleic acid.

### EXAMPLES

### Example 1

### Modified mRNA Production

Modified mRNAs (mmRNA) according to the invention may be made using standard laboratory methods and materials. The open reading frame (ORF) of the gene of interest may be flanked by a 5′ untranslated region (UTR) which may contain a strong Kozak translational initiation signal and/or an alpha-globin 3′ UTR which may include an oligo(dT) sequence for templated addition of a poly-A tail. The modified mRNAs may be modified to reduce the cellular innate immune response. The modifications to reduce the cellular response may include pseudouridine (ψ) and 5-methyl-cytidine (5meC, 5mc or m⁵C). (See, Kariko K et al. Immunity 23:165-75 (2005), Kariko K et al. Mol Ther 16:1833-40 (2008), Anderson B R et al. NAR (2010); each of which are herein incorporated by reference in their entireties).

The ORF may also include various upstream or downstream additions (such as, but not limited to, β-globin, tags, etc.) may be ordered from an optimization service such as, but limited to, DNA2.0 (Menlo Park, Calif.) and may contain multiple cloning sites which may have XbaI recognition. Upon receipt of the construct, it may be reconstituted and transformed into chemically competent E. coli.

For the present invention, NEB DH5-alpha Competent E. coli are used. Transformations are performed according to NEB instructions using 100 ng of plasmid. The protocol is as follows: Thaw a tube of NEB 5-alpha Competent E. coli cells on ice for 10 minutes. Add 1-5 μl containing 1 pg-100 ng of plasmid DNA to the cell mixture. Carefully flick the tube 4-5 times to mix cells and DNA. Do not vortex.
1. Place the mixture on ice for 30 minutes. Do not mix.
2. Heat shock at 42° C. for exactly 30 seconds. Do not mix.
3. Place on ice for 5 minutes. Do not mix.
4. Pipette 950 μl of room temperature SOC into the mixture.
5. Place at 37° C. for 60 minutes. Shake vigorously (250 rpm) or rotate.
6. Warm selection plates to 37° C.
7. Mix the cells thoroughly by flicking the tube and inverting.
8. Spread 50-100 μl of each dilution onto a selection plate and incubate overnight at 37° C.

Alternatively, incubate at 30° C. for 24-36 hours or 25° C. for 48 hours.

A single colony is then used to inoculate 5 ml of LB growth media using the appropriate antibiotic and then allowed to grow (250 RPM, 37° C.) for 5 hours. This is then used to inoculate a 200 ml culture medium and allowed to grow overnight under the same conditions.

To isolate the plasmid (up to 850 μg), a maxi prep is performed using the Invitrogen PURELINK™ HiPure Maxiprep Kit (Carlsbad, Calif.), following the manufacturer's instructions.

In order to generate cDNA for In Vitro Transcription (IVT), the plasmid first linearized using a restriction enzyme such as XbaI. A typical restriction digest with XbaI will comprise the following: Plasmid 1.0 μg; 10× Buffer 1.0 μl; XbaI 1.5 μl; dH₂0 up to 10 μl; incubated at 37° C. for 1 hr. If performing at lab scale (<5 μg), the reaction is cleaned up using Invitrogen's PURELINK™ PCR Micro Kit (Carlsbad, Calif.) per manufacturer's instructions. Larger scale purifications may need to be done with a product that has a larger load capacity such as Invitrogen's standard PURELINK™ PCR Kit (Carlsbad, Calif.). Following the cleanup, the linearized vector is quantified using the NanoDrop and analyzed to confirm linearization using agarose gel electrophoresis.

As a non-limiting example, G-CSF may represent the polypeptide of interest. Sequences used in the steps outlined in Examples 1-5 are shown in Table 6. It should be noted that the start codon (ATG or AUG) has been underlined in SEQ ID NO: 174 and 175 in Table 6.

TABLE 6
G-CSF Sequences

| SEQ | |
|---|---|
| ID NO | Description |
| 174 | G-CSF cDNA containing T7 polymerase |
| | site, AfeI and Xba restriction site: |
| | TAATACGACTCACTATAGGGAAATAAGAGAGAAAAGAAGAGTA |
| | AGAAGAAATATAAGAGCCACCATGGCCGGTCCCGCGACCCAAA |
| | GCCCCATGAAACTTATGGCCCTGCAGTTGCTGCTTTGGCACTC |
| | GGCCCTCTGGACAGTCCAAGAAGCGACTCCTCTCGGACCTGCC |
| | TCATCGTTGCCGCAGTCATTCCTTTTGAAGTGTCTGGAGCAGG |
| | TGCGAAAGATTCAGGGCGATGGAGCCGCACTCCAAGAGAAGCT |
| | CTGCGCGACATACAAACTTTGCCATCCCGAGGAGCTCGTACTG |
| | CTCGGGCACAGCTTGGGGATTCCCTGGGCTCCTCTCTCGTCCT |
| | GTCCGTCGCAGGCTTTGCAGTTGGCAGGGTGCCTTTCCCAGCT |
| | CCACTCCGGTTTGTTCTTGTATCAGGGACTGCTGCAAGCCCTT |
| | GAGGGAATCTCGCCAGAATTGGGCCCGACGCTGGACACGTTGC |
| | AGCTCGACGTGGCGGATTTCGCAACAACCATCTGGCAGCAGAT |
| | GGAGGAACTGGGGATGGCACCCGCGCTGCAGCCCACGCAGGGG |
| | GCAATGCCGGCCTTTGCGTCCGCGTTTCAGCGCAGGGCGGGTG |
| | GAGTCCTCGTAGCGAGCCACCTTCAATCATTTTTGGAAGTCTC |
| | GTACCGGGTGCTGAGACATCTTGCGCAGCCGTGAAGCGCTGCC |
| | TTCTGCGGGGCTTGCCTTCTGGCCATGCCCTTCTTCTCTCCCT |
| | TGCACCTGTACCTCTTGGTCTTTGAATAAAGCCTGAGTAGGAA |
| | GGCGGCCGCTCGAGCATGCATCTAGA |
| 175 | G-CSF mRNA: |
| | GGGAAAUAAGAGAGAAAAGAAGAGUAAGAAGAAAUAUAAGAGC |
| | CACCAUGGCCGGUCCCGCGACCCAAAGCCCCAUGAAACUUAUG |
| | GCCCUGCAGUUGCUGCUUUGGCACUCGGCCCUCUGGACAGUCC |
| | AAGAAGCGACUCCUCUCGGACCUGCCUCAUCGUUGCCGCAGUC |
| | AUUCCUUUUGAAGUGUCUGGAGCAGGUGCGAAAGAUUCAGGGC |
| | GAUGGAGCCGCACUCCAAGAGAAGCUCUGCGCGACAUACAAAC |
| | UUUGCCAUCCCGAGGAGCUCGUACUGCUCGGGCACAGCUUGGG |
| | GAUUCCCUGGGCUCCUCUCUCGUCCUGUCCGUCGCAGGCUUUG |
| | CAGUUGGCAGGGUGCCUUUCCCAGCUCCACUCCGGUUUGUUCU |
| | UGUAUCAGGGACUGCUGCAAGCCCUUGAGGGAAUCUCGCCAGA |
| | AUUGGGCCCGACGCUGGACACGUUGCAGCUCGACGUGGCGGAU |
| | UUCGCAACAACCAUCUGGCAGCAGAUGGAGGAACUGGGGAUGG |
| | CACCCGCGCUGCAGCCCACGCAGGGGGCAAUGCCGGCCUUUGC |
| | GUCCGCGUUUCAGCGCAGGGCGGGUGGAGUCCUCGUAGCGAGC |
| | CACCUUCAAUCAUUUUUGGAAGUCUCGUACCGGGUGCUGAGAC |
| | AUCUUGCGCAGCCGUGAAGCGCUGCCUUCUGCGGGGCUUGCCU |
| | UCUGGCCAUGCCCUUCUUCUCUCCCUUGCACCUGUACCUCUUG |
| | GUCUUUGAAUAAAGCCUGAGUAGGAAG |
| 176 | G-CSF Protein: |
| | MAGPATQSPMKLMALQLLLWHSALWTVQEATPLGPASSLPQSF |
| | LLKCLEQVRKIQGDGAALQEKLVSECATYKLCHPEELVLLGHS |
| | LGIPWAPLSSCPSQALQLAGCLSQLHSGLFLYQGLLQALEGIS |
| | PELGPTLDTLQLDVADFATTIWQQMEELGMAPALQPTQGAMPA |
| | FASAFQRRAGGVLVASHLQSFLEVSYRVLRHLAQP |

### Example 2

### PCR for cDNA Production

PCR procedures for the preparation of cDNA are performed using 2× KAPA HIFI™ HotStart ReadyMix by Kapa Biosystems (Woburn, Mass.). This system includes 2× KAPA ReadyMix 12.5 μl; Forward Primer (10 uM) 0.75 μl; Reverse Primer (10 uM) 0.75 μl; Template cDNA 100 ng; and dH₂0 diluted to 25.0 μl. The reaction conditions are at 95° C. for 5 min. and 25 cycles of 98° C. for 20 sec, then 58° C. for 15 sec, then 72° C. for 45 sec, then 72° C. for 5 min. then 4° C. to termination.

The reverse primer of the instant invention incorporates a poly-T₁₂₀ for a poly-A₁₂₀ in the mRNA. Other reverse primers with longer or shorter poly(T) tracts can be used to adjust the length of the poly(A) tail in the mRNA.

The reaction is cleaned up using Invitrogen's PURELINK™ PCR Micro Kit (Carlsbad, Calif.) per manufacturer's instructions (up to 5 μg). Larger reactions will require a cleanup using a product with a larger capacity. Following the cleanup, the cDNA is quantified using the NanoDrop and analyzed by agarose gel electrophoresis to confirm the cDNA is the expected size. The cDNA is then submitted for sequencing analysis before proceeding to the in vitro transcription reaction.

### Example 3

### In Vitro Transcription (IVT)

The in vitro transcription reaction generates mRNA containing modified nucleotides or modified RNA. The input nucleotide triphosphate (NTP) mix is made in-house using natural and un-natural NTPs.

A typical in vitro transcription reaction includes the following:

| | | | |
|---|---|---|---|
| 1. | Template cDNA | 1.0 | μg |
| 2. | 10x transcription buffer (400 mM Tris-HCl | 2.0 | μl |
| | pH 8.0, 190 mM MgCl₂, 50 mM DTT, | | |
| | 10 mM Spermidine) | | |
| 3. | Custom NTPs (25 mM each) | 7.2 | μl |
| 4. | RNase Inhibitor | 20 | U |
| 5. | T7 RNA polymerase | 3000 | U |

| | | |
|---|---|---|
| 6. | dH₂0 | Up to 20.0 μl. and |
| 7. | Incubation at 37° C. for 3 hr-5 hrs. | |

The crude IVT mix may be stored at 4° C. overnight for cleanup the next day. 1 U of RNase-free DNase is then used to digest the original template. After 15 minutes of incubation at 37° C., the mRNA is purified using Ambion's MEGACLEAR™ Kit (Austin, Tex.) following the manufacturer's instructions. This kit can purify up to 500 μg of RNA. Following the cleanup, the RNA is quantified using the NanoDrop and analyzed by agarose gel electrophoresis to confirm the RNA is the proper size and that no degradation of the RNA has occurred.

### Example 4

### Enzymatic Capping of mRNA

Capping of the mRNA is performed as follows where the mixture includes: IVT RNA 60 μg-180 μg and dH₂0 up to 72 μl. The mixture is incubated at 65° C. for 5 minutes to denature RNA, and then is transferred immediately to ice.

The protocol then involves the mixing of 10× Capping Buffer (0.5 M Tris-HCl (pH 8.0), 60 mM KCl, 12.5 mM MgCl₂) (10.0 μl); 20 mM GTP (5.0 μl); 20 mM S-Adenosyl Methionine (2.5 μl); RNase Inhibitor (100 U); 2′-O-Methyltransferase (400U); Vaccinia capping enzyme (Guanylyl transferase) (40 U); dH₂0 (Up to 28 μl); and incubation at 37° C. for 30 minutes for 60 μg RNA or up to 2 hours for 180 μg of RNA.

The mRNA is then purified using Ambion's MEGACLEAR™ Kit (Austin, Tex.) following the manufacturer's instructions. Following the cleanup, the RNA is quantified using the NANODROP™ (ThermoFisher, Waltham, Mass.) and analyzed by agarose gel electrophoresis to confirm the RNA is the proper size and that no degradation of the RNA has occurred. The RNA product may also be sequenced by running a reverse-transcription-PCR to generate the cDNA for sequencing.

### Example 5

### PolyA Tailing Reaction

Without a poly-T in the cDNA, a poly-A tailing reaction must be performed before cleaning the final product. This is done by mixing Capped IVT RNA (100 μl); RNase Inhibitor (20 U); 10× Tailing Buffer (0.5 M Tris-HCl (pH 8.0), 2.5 M NaCl, 100 mM MgCl₂)(12.0 μl); 20 mM ATP (6.0 μl); Poly-A Polymerase (20 U); dH₂0 up to 123.5 μl and incubation at 37° C. for 30 min. If the poly-A tail is already in the transcript, then the tailing reaction may be skipped and proceed directly to cleanup with Ambion's MEGACLEAR™ kit (Austin, Tex.) (up to 500 μg). Poly-A Polymerase is preferably a recombinant enzyme expressed in yeast.

For studies performed and described herein, the poly-A tail is encoded in the IVT template to comprise 160 nucleotides in length. However, it should be understood that the processivity or integrity of the polyA tailing reaction may not always result in exactly 160 nucleotides. Hence polyA tails of approximately 160 nucleotides, e.g, about 150-165, 155, 156, 157, 158, 159, 160, 161, 162, 163, 164 or 165 are within the scope of the invention.

### Example 6

### Natural 5′ Caps and 5′ Cap Analogues

5′-capping of modified RNA may be completed concomitantly during the in vitro-transcription reaction using the following chemical RNA cap analogs to generate the 5′-guanosine cap structure according to manufacturer protocols: 3″-O-Me-m7G(5)ppp(5′) G [the ARCA cap]; G(5)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes are preferably derived from a recombinant source.

When transfected into mammalian cells, the modified mRNAs have a stability of between 12-18 hours or more than 18 hours, e.g., 24, 36, 48, 60, 72 or greater than 72 hours.

### Example 7

### Capping

A. Protein Expression Assay

Synthetic mRNAs encoding human G-CSF (mRNA sequence fully modified with 5-methylcytosine at each cytosine and pseudouridine replacement at each uridine site shown in SEQ ID NO: 175 with a polyA tail approximately 160 nucleotides in length not shown in sequence) containing the ARCA (3′ O-Me-m7G(5′)ppp(5′)G) cap analog or the Cap1 structure can be transfected into human primary keratinocytes at equal concentrations. 6, 12, 24 and 36 hours post-transfection the amount of G-CSF secreted into the culture medium can be assayed by ELISA. Synthetic mRNAs that secrete higher levels of G-CSF into the medium would correspond to a synthetic mRNA with a higher translationally-competent Cap structure.

B. Purity Analysis Synthesis

Synthetic mRNAs encoding human G-CSF (mRNA sequence fully modified with 5-methylcytosine at each cytosine and pseudouridine replacement at each uridine site shown in SEQ ID NO: 175 with a polyA tail approximately 160 nucleotides in length not shown in sequence) containing the ARCA cap analog or the Cap1 structure crude synthesis products can be compared for purity using denaturing Agarose-Urea gel electrophoresis or HPLC analysis. Synthetic mRNAs with a single, consolidated band by electrophoresis correspond to the higher purity product compared to a synthetic mRNA with multiple bands or streaking bands. Synthetic mRNAs with a single HPLC peak would also correspond to a higher purity product. The capping reaction with a higher efficiency would provide a more pure mRNA population.

C. Cytokine Analysis

Synthetic mRNAs encoding human G-CSF (mRNA sequence fully modified with 5-methylcytosine at each cytosine and pseudouridine replacement at each uridine site shown in SEQ ID NO: 175 with a polyA tail approximately 160 nucleotides in length not shown in sequence) containing the ARCA cap analog or the Cap1 structure can be transfected into human primary keratinocytes at multiple concentrations. 6, 12, 24 and 36 hours post-transfection the amount of pro-inflammatory cytokines such as TNF-alpha and IFN-beta secreted into the culture medium can be assayed by ELISA. Synthetic mRNAs that secrete higher levels of pro-inflammatory cytokines into the medium would correspond to a synthetic mRNA containing an immune-activating cap structure.

D. Capping Reaction Efficiency

Synthetic mRNAs encoding human G-CSF (mRNA sequence fully modified with 5-methylcytosine at each cytosine and pseudouridine replacement at each uridine site shown in SEQ ID NO: 175 with a polyA tail approximately 160 nucleotides in length not shown in sequence) containing the ARCA cap analog or the Cap1 structure can be analyzed for capping reaction efficiency by LC-MS after capped mRNA nuclease treatment. Nuclease treatment of capped mRNAs would yield a mixture of free nucleotides and the capped 5′-5-triphosphate cap structure detectable by LC-MS. The amount of capped product on the LC-MS spectra can be expressed as a percent of total mRNA from the reaction and would correspond to capping reaction efficiency. The cap structure with higher capping reaction efficiency would have a higher amount of capped product by LC-MS.

### Example 8

### Agarose Gel Electrophoresis of Modified RNA or RT PCR Products

Individual modified RNAs (200-400 ng in a 20 μl volume) or reverse transcribed PCR products (200-400 ng) are loaded into a well on a non-denaturing 1.2% Agarose E-Gel (Invitrogen, Carlsbad, Calif.) and run for 12-15 minutes according to the manufacturer protocol.

### Example 9

### Nanodrop Modified RNA Quantification and UV Spectral Data

Modified RNAs in TE buffer (1 μl) are used for Nanodrop UV absorbance readings to quantitate the yield of each modified RNA from an in vitro transcription reaction.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

### Example 10

### In Vitro Transfection of VEGF-A

Human vascular endothelial growth factor-isoform A (VEGF-A) modified mRNA (mRNA sequence shown in SEQ ID NO: 177; poly-A tail of approximately 160 nucleotides not shown in sequence; 5′ cap, Cap1) was transfected via reverse transfection in Human Keratinocyte cells in 24 multi-well plates. Human Keratinocytes cells were grown in EPILIFE® medium with Supplement S7 from Invitrogen (Carlsbad, Calif.) until they reached a confluence of 50-70%. The cells were transfected with 0, 46.875, 93.75, 187.5, 375, 750, and 1500 ng of modified mRNA (mmRNA) encoding VEGF-A which had been complexed with RNAIMAX™ from Invitrogen (Carlsbad, Calif.). The RNA:RNAIMAX™ complex was formed by first incubating the RNA with Supplement-free EPILIFE® media in a 5× volumetric dilution for 10 minutes at room temperature. In a second vial, RNAIMAX′ reagent was incubated with Supplement-free EPILIFE® Media in a 10× volumetric dilution for 10 minutes at room temperature. The RNA vial was then mixed with the RNAIMAX′ vial and incubated for 20-30 minutes at room temperature before being added to the cells in a drop-wise fashion.

The fully optimized mRNA encoding VEGF-A transfected with the Human Keratinocyte cells included modifications during translation such as natural nucleoside triphosphates (NTP), pseudouridine at each uridine site and 5-methylcytosine at each cytosine site (pseudo-U/5mC), and N1-methyl-pseudouridine at each uridine site and 5-methylcytosine at each cytosine site (N1-methyl-Pseudo-U/5mC). Cells were transfected with the mmRNA encoding VEGF-A and secreted VEGF-A concentration (ρg/ml) in the culture medium was measured at 6, 12, 24, and 48 hours post-transfection for each of the concentrations using an ELISA kit from Invitrogen (Carlsbad, Calif.) following the manufacturers recommended instructions. These data, shown in Table 7, show that modified mRNA encoding VEGF-A is capable of being translated in Human Keratinocyte cells and that VEGF-A is transported out of the cells and released into the extracellular environment.

TABLE 7
VEGF-A Dosing and Protein Secretion

| | | | | |
|---|---|---|---|---|
| | 6 hours | 12 hours | 24 hours | 48 hours |
| Dose (ng) | (pg/ml) | (pg/ml) | (pg/ml) | (pg/ml) |

VEGF-A Dose Containing Natural NTPs

| | | | | |
|---|---|---|---|---|
| 46.875 | 10.37 | 18.07 | 33.90 | 67.02 |
| 93.75 | 9.79 | 20.54 | 41.95 | 65.75 |
| 187.5 | 14.07 | 24.56 | 45.25 | 64.39 |
| 375 | 19.16 | 37.53 | 53.61 | 88.28 |
| 750 | 21.51 | 38.90 | 51.44 | 61.79 |
| 1500 | 36.11 | 61.90 | 76.70 | 86.54 |

VEGF-A Dose Containing Pseudo-U/5mC

| | | | | |
|---|---|---|---|---|
| 46.875 | 10.13 | 16.67 | 33.99 | 72.88 |
| 93.75 | 11.00 | 20.00 | 46.47 | 145.61 |
| 187.5 | 16.04 | 34.07 | 83.00 | 120.77 |
| 375 | 69.15 | 188.10 | 448.50 | 392.44 |
| 750 | 133.95 | 304.30 | 524.02 | 526.58 |
| 1500 | 198.96 | 345.65 | 426.97 | 505.41 |

VEGF-A Dose Containing N1-methyl-Pseudo-U/5mC

| | | | | |
|---|---|---|---|---|
| 46.875 | 0.03 | 6.02 | 27.65 | 100.42 |
| 93.75 | 12.37 | 46.38 | 121.23 | 167.56 |
| 187.5 | 104.55 | 365.71 | 1025.41 | 1056.91 |
| 375 | 605.89 | 1201.23 | 1653.63 | 1889.23 |
| 750 | 445.41 | 1036.45 | 1522.86 | 1954.81 |
| 1500 | 261.61 | 714.68 | 1053.12 | 1513.39 |

```
<160> NUMBER OF SEQ ID NOS: 181
<210> SEQ ID NO 1
<211> LENGTH: 2809
<212> TYPE: DNA
<213> ORGANISM: Homo sapiens
<400> SEQUENCE: 1
acgcgcgccc tgcggagccc gcccaactcc ggcgagccgg gcctgcgcct actcctcctc 60
ctcctctccc ggcggcggct gcggcggagg cgccgactcg gccttgcgcc cgccctcagg 120
cccgcgcggg cggcgcagcg aggccccggg cggcgggtgg tggctgccag gcggctcggc 180
cgcgggcgct gcccggcccc ggcgagcgga gggcggagcg cggcgccgga gccgagggcg 240
cgccgcggag ggggtgctgg gccgcgctgt gcccggccgg gcggcggctg caagaggagg 300
ccggaggcga gcgcggggcc ggcggtgggc gcgcagggcg gctcgcagct cgcagccggg 360
gccgggccag gcgtccaggc aggtgatcgg tgtggcggcg gcggcggcgg cggccccaga 420
ctccctccgg agttcttctt ggggctgatg tccgcaaata tgcagaatta ccggccgggt 480
cgctcctgaa gccagcgcgg ggagcgagcg cggcggcggc cagcaccggg aacgcaccga 540
ggaagaagcc cagcccccgc cctccgcccc ttccgtcccc accccctacc cggcggccca 600
ggaggctccc cgcgctgcgg gcgcgcactc cctgtttctc ctcctcctgg ctggcgctgc 660
ctgcctctcc gcactcactg ctcgcgccgg gcgcgctccg ccagctccgt gctccccgcg 720
ccaccctcct ccgggccgcg ctccctaagg gatggtactg aatttcgccg ccacaggaga 780
ccggctggag cgcccgcccc gcggcctcgc ctctcctccg agcagccagc gcctcgggac 840
gcgatgagga ccttggcttg cctgctgctc ctcggctgcg gatacctcgc ccatgttctg 900
gccgaggaag ccgagatccc ccgcgaggtg atcgagaggc tggcccgcag tcagatccac 960
agcatccggg acctccagcg actcctggag atagactccg tagggagtga ggattctttg 1020
gacaccagcc tgagagctca cggggtccat gccactaagc atgtgcccga gaagcggccc 1080
ctgcccattc ggaggaagag aagcatcgag gaagctgtcc ccgctgtctg caagaccagg 1140
acggtcattt acgagattcc tcggagtcag gtcgacccca cgtccgccaa cttcctgatc 1200
tggcccccgt gcgtggaggt gaaacgctgc accggctgct gcaacacgag cagtgtcaag 1260
tgccagccct cccgcgtcca ccaccgcagc gtcaaggtgg ccaaggtgga atacgtcagg 1320
aagaagccaa aattaaaaga agtccaggtg aggttagagg agcatttgga gtgcgcctgc 1380
gcgaccacaa gcctgaatcc ggattatcgg gaagaggaca cgggaaggcc tagggagtca 1440
ggtaaaaaac ggaaaagaaa aaggttaaaa cccacctaaa gcagccaacc agatgtgagg 1500
tgaggatgag ccgcagccct ttcctgggac atggatgtac atggcgtgtt acattcctga 1560
acctactatg tacggtgctt tattgccagt gtgcggtctt tgttctcctc cgtgaaaaac 1620
tgtgtccgag aacactcggg agaacaaaga gacagtgcac atttgtttaa tgtgacatca 1680
aagcaagtat tgtagcactc ggtgaagcag taagaagctt ccttgtcaaa aagagagaga 1740
gagaaagaga gagagaaaac aaaaccacaa atgacaaaaa caaaacggac tcacaaaaat 1800
atctaaactc gatgagatgg agggtcgccc cgtgggatgg aagtgcagag gtctcagcag 1860
actggatttc tgtccgggtg gtcacaggtg cttttttgcc gaggatgcag agcctgcttt 1920
gggaacgact ccagaggggt gctggtgggc tctgcagggg cccgcaggaa gcaggaatgt 1980
cttggaaacc gccacgcgaa ctttagaaac cacacctcct cgctgtagta tttaagccca 2040
tacagaaacc ttcctgagag ccttaagtgg tttttttttt tgtttttgtt ttgttttttt 2100
tttttttgtt tttttttttt tttttttaca ccataaagtg attattaagc tttccttttt 2160
actctttggc tagctttttt tttttttttt tttttttaat tatctcttgg atgacattta 2220
caccgataac acacaggctg ctgtaactgt caggacagtg cgacggtatt tttcctagca 2280
agatgcaaac taatgagatg tattaaaata aacatggtat acctacctat gcatcatttc 2340
ctaaatgttt ctggctttgt gtttctccct taccctgctt tatttgttaa tttaagccat 2400
tttgaaagaa ctatgcgtca accaatcgta cgccgtccct gcggcacctg ccccagagcc 2460
cgtttgtggc tgagtgacaa cttgttcccc gcagtgcaca cctagaatgc tgtgttccca 2520
cgcggcacgt gagatgcatt gccgcttctg tctgtgttgt tggtgtgccc tggtgccgtg 2580
gtggcggtca ctccctctgc tgccagtgtt tggacagaac ccaaattctt tatttttggt 2640
aagatattgt gctttacctg tattaacaga aatgtgtgtg tgtggtttgt ttttttgtaa 2700
aggtgaagtt tgtatgttta cctaatatta cctgttttgt atacctgaga gcctgctatg 2760
ttcttttttt gttgatccaa aattaaaaaa aaaaatacca ccaacaaaa 2809
<210> SEQ ID NO 2
<211> LENGTH: 2740
<212> TYPE: DNA
<213> ORGANISM: Homo sapiens
<400> SEQUENCE: 2
acgcgcgccc tgcggagccc gcccaactcc ggcgagccgg gcctgcgcct actcctcctc 60
ctcctctccc ggcggcggct gcggcggagg cgccgactcg gccttgcgcc cgccctcagg 120
cccgcgcggg cggcgcagcg aggccccggg cggcgggtgg tggctgccag gcggctcggc 180
cgcgggcgct gcccggcccc ggcgagcgga gggcggagcg cggcgccgga gccgagggcg 240
cgccgcggag ggggtgctgg gccgcgctgt gcccggccgg gcggcggctg caagaggagg 300
ccggaggcga gcgcggggcc ggcggtgggc gcgcagggcg gctcgcagct cgcagccggg 360
gccgggccag gcgtccaggc aggtgatcgg tgtggcggcg gcggcggcgg cggccccaga 420
ctccctccgg agttcttctt ggggctgatg tccgcaaata tgcagaatta ccggccgggt 480
cgctcctgaa gccagcgcgg ggagcgagcg cggcggcggc cagcaccggg aacgcaccga 540
ggaagaagcc cagcccccgc cctccgcccc ttccgtcccc accccctacc cggcggccca 600
ggaggctccc cgcgctgcgg gcgcgcactc cctgtttctc ctcctcctgg ctggcgctgc 660
ctgcctctcc gcactcactg ctcgcgccgg gcgcgctccg ccagctccgt gctccccgcg 720
ccaccctcct ccgggccgcg ctccctaagg gatggtactg aatttcgccg ccacaggaga 780
ccggctggag cgcccgcccc gcggcctcgc ctctcctccg agcagccagc gcctcgggac 840
gcgatgagga ccttggcttg cctgctgctc ctcggctgcg gatacctcgc ccatgttctg 900
gccgaggaag ccgagatccc ccgcgaggtg atcgagaggc tggcccgcag tcagatccac 960
agcatccggg acctccagcg actcctggag atagactccg tagggagtga ggattctttg 1020
gacaccagcc tgagagctca cggggtccat gccactaagc atgtgcccga gaagcggccc 1080
ctgcccattc ggaggaagag aagcatcgag gaagctgtcc ccgctgtctg caagaccagg 1140
acggtcattt acgagattcc tcggagtcag gtcgacccca cgtccgccaa cttcctgatc 1200
tggcccccgt gcgtggaggt gaaacgctgc accggctgct gcaacacgag cagtgtcaag 1260
tgccagccct cccgcgtcca ccaccgcagc gtcaaggtgg ccaaggtgga atacgtcagg 1320
aagaagccaa aattaaaaga agtccaggtg aggttagagg agcatttgga gtgcgcctgc 1380
gcgaccacaa gcctgaatcc ggattatcgg gaagaggaca cggatgtgag gtgaggatga 1440
gccgcagccc tttcctggga catggatgta catggcgtgt tacattcctg aacctactat 1500
gtacggtgct ttattgccag tgtgcggtct ttgttctcct ccgtgaaaaa ctgtgtccga 1560
gaacactcgg gagaacaaag agacagtgca catttgttta atgtgacatc aaagcaagta 1620
ttgtagcact cggtgaagca gtaagaagct tccttgtcaa aaagagagag agagaaagag 1680
agagagaaaa caaaaccaca aatgacaaaa acaaaacgga ctcacaaaaa tatctaaact 1740
cgatgagatg gagggtcgcc ccgtgggatg gaagtgcaga ggtctcagca gactggattt 1800
ctgtccgggt ggtcacaggt gcttttttgc cgaggatgca gagcctgctt tgggaacgac 1860
tccagagggg tgctggtggg ctctgcaggg gcccgcagga agcaggaatg tcttggaaac 1920
cgccacgcga actttagaaa ccacacctcc tcgctgtagt atttaagccc atacagaaac 1980
cttcctgaga gccttaagtg gttttttttt ttgtttttgt tttgtttttt ttttttttgt 2040
tttttttttt ttttttttac accataaagt gattattaag ctttcctttt tactctttgg 2100
ctagcttttt tttttttttt ttttttttaa ttatctcttg gatgacattt acaccgataa 2160
cacacaggct gctgtaactg tcaggacagt gcgacggtat ttttcctagc aagatgcaaa 2220
ctaatgagat gtattaaaat aaacatggta tacctaccta tgcatcattt cctaaatgtt 2280
tctggctttg tgtttctccc ttaccctgct ttatttgtta atttaagcca ttttgaaaga 2340
actatgcgtc aaccaatcgt acgccgtccc tgcggcacct gccccagagc ccgtttgtgg 2400
ctgagtgaca acttgttccc cgcagtgcac acctagaatg ctgtgttccc acgcggcacg 2460
tgagatgcat tgccgcttct gtctgtgttg ttggtgtgcc ctggtgccgt ggtggcggtc 2520
actccctctg ctgccagtgt ttggacagaa cccaaattct ttatttttgg taagatattg 2580
tgctttacct gtattaacag aaatgtgtgt gtgtggtttg tttttttgta aaggtgaagt 2640
ttgtatgttt acctaatatt acctgttttg tatacctgag agcctgctat gttctttttt 2700
tgttgatcca aaattaaaaa aaaaaatacc accaacaaaa 2740
<210> SEQ ID NO 3
<211> LENGTH: 3393
<212> TYPE: DNA
<213> ORGANISM: Homo sapiens
<400> SEQUENCE: 3
cctgcctgcc tccctgcgca cccgcagcct cccccgctgc ctccctaggg ctcccctccg 60
gccgccagcg cccatttttc attccctaga tagagatact ttgcgcgcac acacatacat 120
acgcgcgcaa aaaggaaaaa aaaaaaaaaa agcccaccct ccagcctcgc tgcaaagaga 180
aaaccggagc agccgcagct cgcagctcgc agctcgcagc ccgcagcccg cagaggacgc 240
ccagagcggc gagcgggcgg gcagacggac cgacggactc gcgccgcgtc cacctgtcgg 300
ccgggcccag ccgagcgcgc agcgggcacg ccgcgcgcgc ggagcagccg tgcccgccgc 360
ccgggccccg cgccagggcg cacacgctcc cgccccccta cccggcccgg gcgggagttt 420
gcacctctcc ctgcccgggt gctcgagctg ccgttgcaaa gccaactttg gaaaaagttt 480
tttgggggag acttgggcct tgaggtgccc agctccgcgc tttccgattt tgggggcctt 540
tccagaaaat gttgcaaaaa agctaagccg gcgggcagag gaaaacgcct gtagccggcg 600
agtgaagacg aaccatcgac tgccgtgttc cttttcctct tggaggttgg agtcccctgg 660
gcgcccccac acggctagac gcctcggctg gttcgcgacg cagccccccg gccgtggatg 720
ctcactcggg ctcgggatcc gcccaggtag cggcctcgga cccaggtcct gcgcccaggt 780
cctcccctgc cccccagcga cggagccggg gccgggggcg gcggcgcccg ggggccatgc 840
gggtgagccg cggctgcaga ggcctgagcg cctgatcgcc gcggacccga gccgagccca 900
cccccctccc cagcccccca ccctggccgc gggggcggcg cgctcgatct acgcgtccgg 960
ggccccgcgg ggccgggccc ggagtcggca tgaatcgctg ctgggcgctc ttcctgtctc 1020
tctgctgcta cctgcgtctg gtcagcgccg agggggaccc cattcccgag gagctttatg 1080
agatgctgag tgaccactcg atccgctcct ttgatgatct ccaacgcctg ctgcacggag 1140
accccggaga ggaagatggg gccgagttgg acctgaacat gacccgctcc cactctggag 1200
gcgagctgga gagcttggct cgtggaagaa ggagcctggg ttccctgacc attgctgagc 1260
cggccatgat cgccgagtgc aagacgcgca ccgaggtgtt cgagatctcc cggcgcctca 1320
tagaccgcac caacgccaac ttcctggtgt ggccgccctg tgtggaggtg cagcgctgct 1380
ccggctgctg caacaaccgc aacgtgcagt gccgccccac ccaggtgcag ctgcgacctg 1440
tccaggtgag aaagatcgag attgtgcgga agaagccaat ctttaagaag gccacggtga 1500
cgctggaaga ccacctggca tgcaagtgtg agacagtggc agctgcacgg cctgtgaccc 1560
gaagcccggg gggttcccag gagcagcgag ccaaaacgcc ccaaactcgg gtgaccattc 1620
ggacggtgcg agtccgccgg ccccccaagg gcaagcaccg gaaattcaag cacacgcatg 1680
acaagacggc actgaaggag acccttggag cctaggggca tcggcaggag agtgtgtggg 1740
cagggttatt taatatggta tttgctgtat tgcccccatg gggtccttgg agtgataata 1800
ttgtttccct cgtccgtctg tctcgatgcc tgattcggac ggccaatggt gcttccccca 1860
cccctccacg tgtccgtcca cccttccatc agcgggtctc ctcccagcgg cctccggcgt 1920
cttgcccagc agctcaagaa gaaaaagaag gactgaactc catcgccatc ttcttccctt 1980
aactccaaga acttgggata agagtgtgag agagactgat ggggtcgctc tttgggggaa 2040
acgggctcct tcccctgcac ctggcctggg ccacacctga gcgctgtgga ctgtcctgag 2100
gagccctgag gacctctcag catagcctgc ctgatccctg aacccctggc cagctctgag 2160
gggaggcacc tccaggcagg ccaggctgcc tcggactcca tggctaagac cacagacggg 2220
cacacagact ggagaaaacc cctcccacgg tgcccaaaca ccagtcacct cgtctccctg 2280
gtgcctctgt gcacagtggc ttcttttcgt tttcgttttg aagacgtgga ctcctcttgg 2340
tgggtgtggc cagcacacca agtggctggg tgccctctca ggtgggttag agatggagtt 2400
tgctgttgag gtggctgtag atggtgacct gggtatcccc tgcctcctgc caccccttcc 2460
tccccacact ccactctgat tcacctcttc ctctggttcc tttcatctct ctacctccac 2520
cctgcatttt cctcttgtcc tggcccttca gtctgctcca ccaaggggct cttgaacccc 2580
ttattaaggc cccagatgat cccagtcact cctctctagg gcagaagact agaggccagg 2640
gcagcaaggg acctgctcat catattccaa cccagccacg actgccatgt aaggttgtgc 2700
agggtgtgta ctgcacaagg acattgtatg cagggagcac tgttcacatc atagataaag 2760
ctgatttgta tatttattat gacaatttct ggcagatgta ggtaaagagg aaaaggatcc 2820
ttttcctaat tcacacaaag actccttgtg gactggctgt gcccctgatg cagcctgtgg 2880
cttggagtgg ccaaatagga gggagactgt ggtaggggca gggaggcaac actgctgtcc 2940
acatgacctc catttcccaa agtcctctgc tccagcaact gcccttccag gtgggtgtgg 3000
gacacctggg agaaggtctc caagggaggg tgcagccctc ttgcccgcac ccctccctgc 3060
ttgcacactt ccccatcttt gatccttctg agctccacct ctggtggctc ctcctaggaa 3120
accagctcgt gggctgggaa tgggggagag aagggaaaag atccccaaga ccccctgggg 3180
tgggatctga gctcccacct cccttcccac ctactgcact ttcccccttc ccgccttcca 3240
aaacctgctt ccttcagttt gtaaagtcgg tgattatatt tttgggggct ttccttttat 3300
tttttaaatg taaaatttat ttatattccg tatttaaagt tgtaaaaaaa aataaccaca 3360
aaacaaaacc aaatgaaaaa aaaaaaaaaa aaa 3393
<210> SEQ ID NO 4
<211> LENGTH: 2396
<212> TYPE: DNA
<213> ORGANISM: Homo sapiens
<400> SEQUENCE: 4
agagagagag agagactgac tgagcaggaa tggtgagatg tttatcatgg gcctcgggga 60
ccccattccc gaggagcttt atgagatgct gagtgaccac tcgatccgct cctttgatga 120
tctccaacgc ctgctgcacg gagaccccgg agaggaagat ggggccgagt tggacctgaa 180
catgacccgc tcccactctg gaggcgagct ggagagcttg gctcgtggaa gaaggagcct 240
gggttccctg accattgctg agccggccat gatcgccgag tgcaagacgc gcaccgaggt 300
gttcgagatc tcccggcgcc tcatagaccg caccaacgcc aacttcctgg tgtggccgcc 360
ctgtgtggag gtgcagcgct gctccggctg ctgcaacaac cgcaacgtgc agtgccgccc 420
cacccaggtg cagctgcgac ctgtccaggt gagaaagatc gagattgtgc ggaagaagcc 480
aatctttaag aaggccacgg tgacgctgga agaccacctg gcatgcaagt gtgagacagt 540
ggcagctgca cggcctgtga cccgaagccc ggggggttcc caggagcagc gagccaaaac 600
gccccaaact cgggtgacca ttcggacggt gcgagtccgc cggcccccca agggcaagca 660
ccggaaattc aagcacacgc atgacaagac ggcactgaag gagacccttg gagcctaggg 720
gcatcggcag gagagtgtgt gggcagggtt atttaatatg gtatttgctg tattgccccc 780
atggggtcct tggagtgata atattgtttc cctcgtccgt ctgtctcgat gcctgattcg 840
gacggccaat ggtgcttccc ccacccctcc acgtgtccgt ccacccttcc atcagcgggt 900
ctcctcccag cggcctccgg cgtcttgccc agcagctcaa gaagaaaaag aaggactgaa 960
ctccatcgcc atcttcttcc cttaactcca agaacttggg ataagagtgt gagagagact 1020
gatggggtcg ctctttgggg gaaacgggct ccttcccctg cacctggcct gggccacacc 1080
tgagcgctgt ggactgtcct gaggagccct gaggacctct cagcatagcc tgcctgatcc 1140
ctgaacccct ggccagctct gaggggaggc acctccaggc aggccaggct gcctcggact 1200
ccatggctaa gaccacagac gggcacacag actggagaaa acccctccca cggtgcccaa 1260
acaccagtca cctcgtctcc ctggtgcctc tgtgcacagt ggcttctttt cgttttcgtt 1320
ttgaagacgt ggactcctct tggtgggtgt ggccagcaca ccaagtggct gggtgccctc 1380
tcaggtgggt tagagatgga gtttgctgtt gaggtggctg tagatggtga cctgggtatc 1440
ccctgcctcc tgccacccct tcctccccac actccactct gattcacctc ttcctctggt 1500
tcctttcatc tctctacctc caccctgcat tttcctcttg tcctggccct tcagtctgct 1560
ccaccaaggg gctcttgaac cccttattaa ggccccagat gatcccagtc actcctctct 1620
agggcagaag actagaggcc agggcagcaa gggacctgct catcatattc caacccagcc 1680
acgactgcca tgtaaggttg tgcagggtgt gtactgcaca aggacattgt atgcagggag 1740
cactgttcac atcatagata aagctgattt gtatatttat tatgacaatt tctggcagat 1800
gtaggtaaag aggaaaagga tccttttcct aattcacaca aagactcctt gtggactggc 1860
tgtgcccctg atgcagcctg tggcttggag tggccaaata ggagggagac tgtggtaggg 1920
gcagggaggc aacactgctg tccacatgac ctccatttcc caaagtcctc tgctccagca 1980
actgcccttc caggtgggtg tgggacacct gggagaaggt ctccaaggga gggtgcagcc 2040
ctcttgcccg cacccctccc tgcttgcaca cttccccatc tttgatcctt ctgagctcca 2100
cctctggtgg ctcctcctag gaaaccagct cgtgggctgg gaatggggga gagaagggaa 2160
aagatcccca agaccccctg gggtgggatc tgagctccca cctcccttcc cacctactgc 2220
actttccccc ttcccgcctt ccaaaacctg cttccttcag tttgtaaagt cggtgattat 2280
atttttgggg gctttccttt tattttttaa atgtaaaatt tatttatatt ccgtatttaa 2340
agttgtaaaa aaaaataacc acaaaacaaa accaaatgaa aaaaaaaaaa aaaaaa 2396
<210> SEQ ID NO 5
<211> LENGTH: 3018
<212> TYPE: DNA
<213> ORGANISM: Homo sapiens
<400> SEQUENCE: 5
gcccggagag ccgcatctat tggcagcttt gttattgatc agaaactgct cgccgccgac 60
ttggcttcca gtctggctgc gggcaaccct tgagttttcg cctctgtcct gtcccccgaa 120
ctgacaggtg ctcccagcaa cttgctgggg acttctcgcc gctcccccgc gtccccaccc 180
cctcattcct ccctcgcctt cacccccacc cccaccactt cgccacagct caggatttgt 240
ttaaaccttg ggaaactggt tcaggtccag gttttgcttt gatccttttc aaaaactgga 300
gacacagaag agggctctag gaaaaagttt tggatgggat tatgtggaaa ctaccctgcg 360
attctctgct gccagagcag gctcggcgct tccaccccag tgcagccttc ccctggcggt 420
ggtgaaagag actcgggagt cgctgcttcc aaagtgcccg ccgtgagtga gctctcaccc 480
cagtcagcca aatgagcctc ttcgggcttc tcctgctgac atctgccctg gccggccaga 540
gacaggggac tcaggcggaa tccaacctga gtagtaaatt ccagttttcc agcaacaagg 600
aacagaacgg agtacaagat cctcagcatg agagaattat tactgtgtct actaatggaa 660
gtattcacag cccaaggttt cctcatactt atccaagaaa tacggtcttg gtatggagat 720
tagtagcagt agaggaaaat gtatggatac aacttacgtt tgatgaaaga tttgggcttg 780
aagacccaga agatgacata tgcaagtatg attttgtaga agttgaggaa cccagtgatg 840
gaactatatt agggcgctgg tgtggttctg gtactgtacc aggaaaacag atttctaaag 900
gaaatcaaat taggataaga tttgtatctg atgaatattt tccttctgaa ccagggttct 960
gcatccacta caacattgtc atgccacaat tcacagaagc tgtgagtcct tcagtgctac 1020
ccccttcagc tttgccactg gacctgctta ataatgctat aactgccttt agtaccttgg 1080
aagaccttat tcgatatctt gaaccagaga gatggcagtt ggacttagaa gatctatata 1140
ggccaacttg gcaacttctt ggcaaggctt ttgtttttgg aagaaaatcc agagtggtgg 1200
atctgaacct tctaacagag gaggtaagat tatacagctg cacacctcgt aacttctcag 1260
tgtccataag ggaagaacta aagagaaccg ataccatttt ctggccaggt tgtctcctgg 1320
ttaaacgctg tggtgggaac tgtgcctgtt gtctccacaa ttgcaatgaa tgtcaatgtg 1380
tcccaagcaa agttactaaa aaataccacg aggtccttca gttgagacca aagaccggtg 1440
tcaggggatt gcacaaatca ctcaccgacg tggccctgga gcaccatgag gagtgtgact 1500
gtgtgtgcag agggagcaca ggaggatagc cgcatcacca ccagcagctc ttgcccagag 1560
ctgtgcagtg cagtggctga ttctattaga gaacgtatgc gttatctcca tccttaatct 1620
cagttgtttg cttcaaggac ctttcatctt caggatttac agtgcattct gaaagaggag 1680
acatcaaaca gaattaggag ttgtgcaaca gctcttttga gaggaggcct aaaggacagg 1740
agaaaaggtc ttcaatcgtg gaaagaaaat taaatgttgt attaaataga tcaccagcta 1800
gtttcagagt taccatgtac gtattccact agctgggttc tgtatttcag ttctttcgat 1860
acggcttagg gtaatgtcag tacaggaaaa aaactgtgca agtgagcacc tgattccgtt 1920
gccttgctta actctaaagc tccatgtcct gggcctaaaa tcgtataaaa tctggatttt 1980
tttttttttt tttgctcata ttcacatatg taaaccagaa cattctatgt actacaaacc 2040
tggtttttaa aaaggaacta tgttgctatg aattaaactt gtgtcgtgct gataggacag 2100
actggatttt tcatatttct tattaaaatt tctgccattt agaagaagag aactacattc 2160
atggtttgga agagataaac ctgaaaagaa gagtggcctt atcttcactt tatcgataag 2220
tcagtttatt tgtttcattg tgtacatttt tatattctcc ttttgacatt ataactgttg 2280
gcttttctaa tcttgttaaa tatatctatt tttaccaaag gtatttaata ttctttttta 2340
tgacaactta gatcaactat ttttagcttg gtaaattttt ctaaacacaa ttgttatagc 2400
cagaggaaca aagatgatat aaaatattgt tgctctgaca aaaatacatg tatttcattc 2460
tcgtatggtg ctagagttag attaatctgc attttaaaaa actgaattgg aatagaattg 2520
gtaagttgca aagacttttt gaaaataatt aaattatcat atcttccatt cctgttattg 2580
gagatgaaaa taaaaagcaa cttatgaaag tagacattca gatccagcca ttactaacct 2640
attccttttt tggggaaatc tgagcctagc tcagaaaaac ataaagcacc ttgaaaaaga 2700
cttggcagct tcctgataaa gcgtgctgtg ctgtgcagta ggaacacatc ctatttattg 2760
tgatgttgtg gttttattat cttaaactct gttccataca cttgtataaa tacatggata 2820
tttttatgta cagaagtatg tctcttaacc agttcactta ttgtactctg gcaatttaaa 2880
agaaaatcag taaaatattt tgcttgtaaa atgcttaata tcgtgcctag gttatgtggt 2940
gactatttga atcaaaaatg tattgaatca tcaaataaaa gaatgtggct attttgggga 3000
gaaaattaaa aaaaaaaa 3018
<210> SEQ ID NO 6
<211> LENGTH: 3997
<212> TYPE: DNA
<213> ORGANISM: Homo sapiens
<400> SEQUENCE: 6
tctcaggggc cgcggccggg gctggagaac gctgctgctc cgctcgcctg ccccgctaga 60
ttcggcgctg cccgccccct gcagcctgtg ctgcagctgc cggccaccgg agggggcgaa 120
caaacaaacg tcaacctgtt gtttgtcccg tcaccattta tcagctcagc accacaagga 180
agtgcggcac ccacacgcgc tcggaaagtt cagcatgcag gaagtttggg gagagctcgg 240
cgattagcac agcgacccgg gccagcgcag ggcgagcgca ggcggcgaga gcgcagggcg 300
gcgcggcgtc ggtcccggga gcagaacccg gctttttctt ggagcgacgc tgtctctagt 360
cgctgatccc aaatgcaccg gctcatcttt gtctacactc taatctgcgc aaacttttgc 420
agctgtcggg acacttctgc aaccccgcag agcgcatcca tcaaagcttt gcgcaacgcc 480
aacctcaggc gagatgagag caatcacctc acagacttgt accgaagaga tgagaccatc 540
caggtgaaag gaaacggcta cgtgcagagt cctagattcc cgaacagcta ccccaggaac 600
ctgctcctga catggcggct tcactctcag gagaatacac ggatacagct agtgtttgac 660
aatcagtttg gattagagga agcagaaaat gatatctgta ggtatgattt tgtggaagtt 720
gaagatatat ccgaaaccag taccattatt agaggacgat ggtgtggaca caaggaagtt 780
cctccaagga taaaatcaag aacgaaccaa attaaaatca cattcaagtc cgatgactac 840
tttgtggcta aacctggatt caagatttat tattctttgc tggaagattt ccaacccgca 900
gcagcttcag agaccaactg ggaatctgtc acaagctcta tttcaggggt atcctataac 960
tctccatcag taacggatcc cactctgatt gcggatgctc tggacaaaaa aattgcagaa 1020
tttgatacag tggaagatct gctcaagtac ttcaatccag agtcatggca agaagatctt 1080
gagaatatgt atctggacac ccctcggtat cgaggcaggt cataccatga ccggaagtca 1140
aaagttgacc tggataggct caatgatgat gccaagcgtt acagttgcac tcccaggaat 1200
tactcggtca atataagaga agagctgaag ttggccaatg tggtcttctt tccacgttgc 1260
ctcctcgtgc agcgctgtgg aggaaattgt ggctgtggaa ctgtcaactg gaggtcctgc 1320
acatgcaatt cagggaaaac cgtgaaaaag tatcatgagg tattacagtt tgagcctggc 1380
cacatcaaga ggaggggtag agctaagacc atggctctag ttgacatcca gttggatcac 1440
catgaacgat gtgattgtat ctgcagctca agaccacctc gataagagaa tgtgcacatc 1500
cttacattaa gcctgaaaga acctttagtt taaggagggt gagataagag acccttttcc 1560
taccagcaac caaacttact actagcctgc aatgcaatga acacaagtgg ttgctgagtc 1620
tcagccttgc tttgttaatg ccatggcaag tagaaaggta tatcatcaac ttctatacct 1680
aagaatatag gattgcattt aataatagtg tttgaggtta tatatgcaca aacacacaca 1740
gaaatatatt catgtctatg tgtatataga tcaaatgttt tttttggtat atataaccag 1800
gtacaccaga gcttacatat gtttgagtta gactcttaaa atcctttgcc aaaataaggg 1860
atggtcaaat atatgaaaca tgtctttaga aaatttagga gataaattta tttttaaatt 1920
ttgaaacaca aaacaatttt gaatcttgct ctcttaaaga aagcatcttg tatattaaaa 1980
atcaaaagat gaggctttct tacatataca tcttagttga ttattaaaaa aggaaaaata 2040
tggtttccag agaaaaggcc aatacctaag cattttttcc atgagaagca ctgcatactt 2100
acctatgtgg actataataa cctgtctcca aaaccatgcc ataataatat aagtgcttta 2160
gaaattaaat cattgtgttt tttatgcatt ttgctgaggc atgcttattc atttaacacc 2220
tatctcaaaa acttacttag aaggtttttt attatagtcc tacaaaagac aatgtataag 2280
ctgtaacaga attttgaatt gtttttcttt gcaaaacccc tccacaaaag caaatccttt 2340
caagaatggc atgggcattc tgtatgaacc tttccagatg gtgttcagtg aaagatgtgg 2400
gtagttgaga acttaaaaag tgaacattga aacatcgacg taactggaaa ttaggtggga 2460
tatttgatag gatccatatc taataatgga ttcgaactct ccaaactaca ccaattaatt 2520
taatgtatct tgcttttgtg ttcccgtctt tttgaaatat agacatggat ttataatggc 2580
attttatatt tggcaggcca tcatagatta tttacaacct aaaagctttt gtgtatcaaa 2640
aaaatcacat tttattaatg taaatttcta atcgtatact tgctcactgt tctgatttcc 2700
tgtttctgaa ccaagtaaaa tcagtcctag aggctatggt tcttaatcta tggagcttgc 2760
tttaagaagc cagttgtcaa ttgtggtaac acaagtttgg ccctgctgtc ctactgttta 2820
atagaaaact gttttacatt ggttaatggt atttagagta attttttctc tctgcctcct 2880
ttgtgtctgt tttaaaggag actaactcca ggagtaggaa atgattcatc atcctccaaa 2940
gcaagaggct taagagagaa acaccgaaat tcagatagct cagggactgc taacagagaa 3000
ctacattttt cttattgcct tgaaagttaa aaggaaagca gatttcttca gtgactttgt 3060
ggtcctacta actacaacca gtttgggtga cagggctggt aaagtcccag tgttagatga 3120
gtgacctaaa tatacttaga tttctaagta tggtgctctc aggtccaagt tcaactattc 3180
ttaagcagtg caattcttcc cagttatttg agatgaaaga tctctgctta ttgaagatgt 3240
accttctaaa actttcctaa aagtgtctga tgtttttact caagagggga gtggtaaaat 3300
taaatactct attgttcaat tctctaaaat cccagaacac aatcagaaat agctcaggca 3360
gacactaata attaagaacg ctcttcctct tcataactgc tttgcaagtt tcctgtgaaa 3420
acatcagttt cctgtaccaa agtcaaaatg aacgttacat cactctaacc tgaacagctc 3480
acaatgtagc tgtaaatata aaaaatgaga gtgttctacc cagttttcaa taaaccttcc 3540
aggctgcaat aaccagcaag gttttcagtt aaagccctat ctgcactttt tatttattag 3600
ctgaaatgta agcaggcata ttcactcact tttctttgcc tttcctgaga gttttattaa 3660
aacttctccc ttggttacct gttatctttt gcacttctaa catgtagcca ataaatctat 3720
ttgatagcca tcaaaggaat aaaaagctgg ccgtacaaat tacatttcaa aacaaaccct 3780
aataaatcca catttccgca tggctcattc acctggaata atgcctttta ttgaatatgt 3840
tcttataggg caaaacactt tcataagtag agttttttat gttttttgtc atatcggtaa 3900
catgcagctt tttcctctca tagcattttc tatagcgaat gtaatatgcc tcttatcttc 3960
atgaaaaata aatattgctt ttgaacaaaa ctaaaaa 3997
<210> SEQ ID NO 7
<211> LENGTH: 3979
<212> TYPE: DNA
<213> ORGANISM: Homo sapiens
<400> SEQUENCE: 7
tctcaggggc cgcggccggg gctggagaac gctgctgctc cgctcgcctg ccccgctaga 60
ttcggcgctg cccgccccct gcagcctgtg ctgcagctgc cggccaccgg agggggcgaa 120
caaacaaacg tcaacctgtt gtttgtcccg tcaccattta tcagctcagc accacaagga 180
agtgcggcac ccacacgcgc tcggaaagtt cagcatgcag gaagtttggg gagagctcgg 240
cgattagcac agcgacccgg gccagcgcag ggcgagcgca ggcggcgaga gcgcagggcg 300
gcgcggcgtc ggtcccggga gcagaacccg gctttttctt ggagcgacgc tgtctctagt 360
cgctgatccc aaatgcaccg gctcatcttt gtctacactc taatctgcgc aaacttttgc 420
agctgtcggg acacttctgc aaccccgcag agcgcatcca tcaaagcttt gcgcaacgcc 480
aacctcaggc gagatgactt gtaccgaaga gatgagacca tccaggtgaa aggaaacggc 540
tacgtgcaga gtcctagatt cccgaacagc taccccagga acctgctcct gacatggcgg 600
cttcactctc aggagaatac acggatacag ctagtgtttg acaatcagtt tggattagag 660
gaagcagaaa atgatatctg taggtatgat tttgtggaag ttgaagatat atccgaaacc 720
agtaccatta ttagaggacg atggtgtgga cacaaggaag ttcctccaag gataaaatca 780
agaacgaacc aaattaaaat cacattcaag tccgatgact actttgtggc taaacctgga 840
ttcaagattt attattcttt gctggaagat ttccaacccg cagcagcttc agagaccaac 900
tgggaatctg tcacaagctc tatttcaggg gtatcctata actctccatc agtaacggat 960
cccactctga ttgcggatgc tctggacaaa aaaattgcag aatttgatac agtggaagat 1020
ctgctcaagt acttcaatcc agagtcatgg caagaagatc ttgagaatat gtatctggac 1080
acccctcggt atcgaggcag gtcataccat gaccggaagt caaaagttga cctggatagg 1140
ctcaatgatg atgccaagcg ttacagttgc actcccagga attactcggt caatataaga 1200
gaagagctga agttggccaa tgtggtcttc tttccacgtt gcctcctcgt gcagcgctgt 1260
ggaggaaatt gtggctgtgg aactgtcaac tggaggtcct gcacatgcaa ttcagggaaa 1320
accgtgaaaa agtatcatga ggtattacag tttgagcctg gccacatcaa gaggaggggt 1380
agagctaaga ccatggctct agttgacatc cagttggatc accatgaacg atgtgattgt 1440
atctgcagct caagaccacc tcgataagag aatgtgcaca tccttacatt aagcctgaaa 1500
gaacctttag tttaaggagg gtgagataag agaccctttt cctaccagca accaaactta 1560
ctactagcct gcaatgcaat gaacacaagt ggttgctgag tctcagcctt gctttgttaa 1620
tgccatggca agtagaaagg tatatcatca acttctatac ctaagaatat aggattgcat 1680
ttaataatag tgtttgaggt tatatatgca caaacacaca cagaaatata ttcatgtcta 1740
tgtgtatata gatcaaatgt tttttttggt atatataacc aggtacacca gagcttacat 1800
atgtttgagt tagactctta aaatcctttg ccaaaataag ggatggtcaa atatatgaaa 1860
catgtcttta gaaaatttag gagataaatt tatttttaaa ttttgaaaca caaaacaatt 1920
ttgaatcttg ctctcttaaa gaaagcatct tgtatattaa aaatcaaaag atgaggcttt 1980
cttacatata catcttagtt gattattaaa aaaggaaaaa tatggtttcc agagaaaagg 2040
ccaataccta agcatttttt ccatgagaag cactgcatac ttacctatgt ggactataat 2100
aacctgtctc caaaaccatg ccataataat ataagtgctt tagaaattaa atcattgtgt 2160
tttttatgca ttttgctgag gcatgcttat tcatttaaca cctatctcaa aaacttactt 2220
agaaggtttt ttattatagt cctacaaaag acaatgtata agctgtaaca gaattttgaa 2280
ttgtttttct ttgcaaaacc cctccacaaa agcaaatcct ttcaagaatg gcatgggcat 2340
tctgtatgaa cctttccaga tggtgttcag tgaaagatgt gggtagttga gaacttaaaa 2400
agtgaacatt gaaacatcga cgtaactgga aattaggtgg gatatttgat aggatccata 2460
tctaataatg gattcgaact ctccaaacta caccaattaa tttaatgtat cttgcttttg 2520
tgttcccgtc tttttgaaat atagacatgg atttataatg gcattttata tttggcaggc 2580
catcatagat tatttacaac ctaaaagctt ttgtgtatca aaaaaatcac attttattaa 2640
tgtaaatttc taatcgtata cttgctcact gttctgattt cctgtttctg aaccaagtaa 2700
aatcagtcct agaggctatg gttcttaatc tatggagctt gctttaagaa gccagttgtc 2760
aattgtggta acacaagttt ggccctgctg tcctactgtt taatagaaaa ctgttttaca 2820
ttggttaatg gtatttagag taattttttc tctctgcctc ctttgtgtct gttttaaagg 2880
agactaactc caggagtagg aaatgattca tcatcctcca aagcaagagg cttaagagag 2940
aaacaccgaa attcagatag ctcagggact gctaacagag aactacattt ttcttattgc 3000
cttgaaagtt aaaaggaaag cagatttctt cagtgacttt gtggtcctac taactacaac 3060
cagtttgggt gacagggctg gtaaagtccc agtgttagat gagtgaccta aatatactta 3120
gatttctaag tatggtgctc tcaggtccaa gttcaactat tcttaagcag tgcaattctt 3180
cccagttatt tgagatgaaa gatctctgct tattgaagat gtaccttcta aaactttcct 3240
aaaagtgtct gatgttttta ctcaagaggg gagtggtaaa attaaatact ctattgttca 3300
attctctaaa atcccagaac acaatcagaa atagctcagg cagacactaa taattaagaa 3360
cgctcttcct cttcataact gctttgcaag tttcctgtga aaacatcagt ttcctgtacc 3420
aaagtcaaaa tgaacgttac atcactctaa cctgaacagc tcacaatgta gctgtaaata 3480
taaaaaatga gagtgttcta cccagttttc aataaacctt ccaggctgca ataaccagca 3540
aggttttcag ttaaagccct atctgcactt tttatttatt agctgaaatg taagcaggca 3600
tattcactca cttttctttg cctttcctga gagttttatt aaaacttctc ccttggttac 3660
ctgttatctt ttgcacttct aacatgtagc caataaatct atttgatagc catcaaagga 3720
ataaaaagct ggccgtacaa attacatttc aaaacaaacc ctaataaatc cacatttccg 3780
catggctcat tcacctggaa taatgccttt tattgaatat gttcttatag ggcaaaacac 3840
tttcataagt agagtttttt atgttttttg tcatatcggt aacatgcagc tttttcctct 3900
catagcattt tctatagcga atgtaatatg cctcttatct tcatgaaaaa taaatattgc 3960
ttttgaacaa aactaaaaa 3979
<210> SEQ ID NO 8
<211> LENGTH: 5600
<212> TYPE: DNA
<213> ORGANISM: Homo sapiens
<400> SEQUENCE: 8
aaaaagagaa actgttggga gaggaatcgt atctccatat ttcttctttc agccccaatc 60
caagggttgt agctggaact ttccatcagt tcttcctttc tttttcctct ctaagccttt 120
gccttgctct gtcacagtga agtcagccag agcagggctg ttaaactctg tgaaatttgt 180
cataagggtg tcaggtattt cttactggct tccaaagaaa catagataaa gaaatctttc 240
ctgtggcttc ccttggcagg ctgcattcag aaggtctctc agttgaagaa agagcttgga 300
ggacaacagc acaacaggag agtaaaagat gccccagggc tgaggcctcc gctcaggcag 360
ccgcatctgg ggtcaatcat actcaccttg cccgggccat gctccagcaa aatcaagctg 420
ttttcttttg aaagttcaaa ctcatcaaga ttatgctgct cactcttatc attctgttgc 480
cagtagtttc aaaatttagt tttgttagtc tctcagcacc gcagcactgg agctgtcctg 540
aaggtactct cgcaggaaat gggaattcta cttgtgtggg tcctgcaccc ttcttaattt 600
tctcccatgg aaatagtatc tttaggattg acacagaagg aaccaattat gagcaattgg 660
tggtggatgc tggtgtctca gtgatcatgg attttcatta taatgagaaa agaatctatt 720
gggtggattt agaaagacaa cttttgcaaa gagtttttct gaatgggtca aggcaagaga 780
gagtatgtaa tatagagaaa aatgtttctg gaatggcaat aaattggata aatgaagaag 840
ttatttggtc aaatcaacag gaaggaatca ttacagtaac agatatgaaa ggaaataatt 900
cccacattct tttaagtgct ttaaaatatc ctgcaaatgt agcagttgat ccagtagaaa 960
ggtttatatt ttggtcttca gaggtggctg gaagccttta tagagcagat ctcgatggtg 1020
tgggagtgaa ggctctgttg gagacatcag agaaaataac agctgtgtca ttggatgtgc 1080
ttgataagcg gctgttttgg attcagtaca acagagaagg aagcaattct cttatttgct 1140
cctgtgatta tgatggaggt tctgtccaca ttagtaaaca tccaacacag cataatttgt 1200
ttgcaatgtc cctttttggt gaccgtatct tctattcaac atggaaaatg aagacaattt 1260
ggatagccaa caaacacact ggaaaggaca tggttagaat taacctccat tcatcatttg 1320
taccacttgg tgaactgaaa gtagtgcatc cacttgcaca acccaaggca gaagatgaca 1380
cttgggagcc tgagcagaaa ctttgcaaat tgaggaaagg aaactgcagc agcactgtgt 1440
gtgggcaaga cctccagtca cacttgtgca tgtgtgcaga gggatacgcc ctaagtcgag 1500
accggaagta ctgtgaagat gttaatgaat gtgctttttg gaatcatggc tgtactcttg 1560
ggtgtaaaaa cacccctgga tcctattact gcacgtgccc tgtaggattt gttctgcttc 1620
ctgatgggaa acgatgtcat caacttgttt cctgtccacg caatgtgtct gaatgcagcc 1680
atgactgtgt tctgacatca gaaggtccct tatgtttctg tcctgaaggc tcagtgcttg 1740
agagagatgg gaaaacatgt agcggttgtt cctcacccga taatggtgga tgtagccagc 1800
tctgcgttcc tcttagccca gtatcctggg aatgtgattg ctttcctggg tatgacctac 1860
aactggatga aaaaagctgt gcagcttcag gaccacaacc atttttgctg tttgccaatt 1920
ctcaagatat tcgacacatg cattttgatg gaacagacta tggaactctg ctcagccagc 1980
agatgggaat ggtttatgcc ctagatcatg accctgtgga aaataagata tactttgccc 2040
atacagccct gaagtggata gagagagcta atatggatgg ttcccagcga gaaaggctta 2100
ttgaggaagg agtagatgtg ccagaaggtc ttgctgtgga ctggattggc cgtagattct 2160
attggacaga cagagggaaa tctctgattg gaaggagtga tttaaatggg aaacgttcca 2220
aaataatcac taaggagaac atctctcaac cacgaggaat tgctgttcat ccaatggcca 2280
agagattatt ctggactgat acagggatta atccacgaat tgaaagttct tccctccaag 2340
gccttggccg tctggttata gccagctctg atctaatctg gcccagtgga ataacgattg 2400
acttcttaac tgacaagttg tactggtgcg atgccaagca gtctgtgatt gaaatggcca 2460
atctggatgg ttcaaaacgc cgaagactta cccagaatga tgtaggtcac ccatttgctg 2520
tagcagtgtt tgaggattat gtgtggttct cagattgggc tatgccatca gtaatgagag 2580
taaacaagag gactggcaaa gatagagtac gtctccaagg cagcatgctg aagccctcat 2640
cactggttgt ggttcatcca ttggcaaaac caggagcaga tccctgctta tatcaaaacg 2700
gaggctgtga acatatttgc aaaaagaggc ttggaactgc ttggtgttcg tgtcgtgaag 2760
gttttatgaa agcctcagat gggaaaacgt gtctggctct ggatggtcat cagctgttgg 2820
caggtggtga agttgatcta aagaaccaag taacaccatt ggacatcttg tccaagacta 2880
gagtgtcaga agataacatt acagaatctc aacacatgct agtggctgaa atcatggtgt 2940
cagatcaaga tgactgtgct cctgtgggat gcagcatgta tgctcggtgt atttcagagg 3000
gagaggatgc cacatgtcag tgtttgaaag gatttgctgg ggatggaaaa ctatgttctg 3060
atatagatga atgtgagatg ggtgtcccag tgtgcccccc tgcctcctcc aagtgcatca 3120
acaccgaagg tggttatgtc tgccggtgct cagaaggcta ccaaggagat gggattcact 3180
gtcttgatat tgatgagtgc caactggggg agcacagctg tggagagaat gccagctgca 3240
caaatacaga gggaggctat acctgcatgt gtgctggacg cctgtctgaa ccaggactga 3300
tttgccctga ctctactcca ccccctcacc tcagggaaga tgaccaccac tattccgtaa 3360
gaaatagtga ctctgaatgt cccctgtccc acgatgggta ctgcctccat gatggtgtgt 3420
gcatgtatat tgaagcattg gacaagtatg catgcaactg tgttgttggc tacatcgggg 3480
agcgatgtca gtaccgagac ctgaagtggt gggaactgcg ccacgctggc cacgggcagc 3540
agcagaaggt catcgtggtg gctgtctgcg tggtggtgct tgtcatgctg ctcctcctga 3600
gcctgtgggg ggcccactac tacaggactc agaagctgct atcgaaaaac ccaaagaatc 3660
cttatgagga gtcgagcaga gatgtgagga gtcgcaggcc tgctgacact gaggatggga 3720
tgtcctcttg ccctcaacct tggtttgtgg ttataaaaga acaccaagac ctcaagaatg 3780
ggggtcaacc agtggctggt gaggatggcc aggcagcaga tgggtcaatg caaccaactt 3840
catggaggca ggagccccag ttatgtggaa tgggcacaga gcaaggctgc tggattccag 3900
tatccagtga taagggctcc tgtccccagg taatggagcg aagctttcat atgccctcct 3960
atgggacaca gacccttgaa gggggtgtcg agaagcccca ttctctccta tcagctaacc 4020
cattatggca acaaagggcc ctggacccac cacaccaaat ggagctgact cagtgaaaac 4080
tggaattaaa aggaaagtca agaagaatga actatgtcga tgcacagtat cttttctttc 4140
aaaagtagag caaaactata ggttttggtt ccacaatctc tacgactaat cacctactca 4200
atgcctggag acagatacgt agttgtgctt ttgtttgctc ttttaagcag tctcactgca 4260
gtcttatttc caagtaagag tactgggaga atcactaggt aacttattag aaacccaaat 4320
tgggacaaca gtgctttgta aattgtgttg tcttcagcag tcaatacaaa tagatttttg 4380
tttttgttgt tcctgcagcc ccagaagaaa ttaggggtta aagcagacag tcacactggt 4440
ttggtcagtt acaaagtaat ttctttgatc tggacagaac atttatatca gtttcatgaa 4500
atgattggaa tattacaata ccgttaagat acagtgtagg catttaactc ctcattggcg 4560
tggtccatgc tgatgatttt gcaaaatgag ttgtgatgaa tcaatgaaaa atgtaattta 4620
gaaactgatt tcttcagaat tagatggctt attttttaaa atatttgaat gaaaacattt 4680
tatttttaaa atattacaca ggaggcttcg gagtttctta gtcattactg tccttttccc 4740
ctacagaatt ttccctcttg gtgtgattgc acagaatttg tatgtatttt cagttacaag 4800
attgtaagta aattgcctga tttgttttca ttatagacaa cgatgaattt cttctaatta 4860
tttaaataaa atcaccaaaa acataaacat tttattgtat gcctgattaa gtagttaatt 4920
atagtctaag gcagtactag agttgaacca aaatgatttg tcaagcttgc tgatgtttct 4980
gtttttcgtt tttttttttt ttccggagag aggataggat ctcactctgt tatccaggct 5040
ggagtgtgca atggcacaat catagctcag tgcagcctca aactcctggg ctcaagcaat 5100
cctcctgcct cagcctcccg agtaactagg accacaggca caggccacca tgcctggcta 5160
aggtttttat ttttattttt tgtagacatg gggatcacac aatgttgccc aggctggtct 5220
tgaactcctg gcctcaagca aggtcgtgct ggtaattttg caaaatgaat tgtgattgac 5280
tttcagcctc ccaacgtatt agattatagg cattagccat ggtgcccagc cttgtaactt 5340
ttaaaaaaat tttttaatct acaactctgt agattaaaat ttcacatggt gttctaatta 5400
aatatttttc ttgcagccaa gatattgtta ctacagataa cacaacctga tatggtaact 5460
ttaaattttg ggggctttga atcattcagt ttatgcatta actagtccct ttgtttatct 5520
ttcatttctc aaccccttgt actttggtga taccagacat cagaataaaa agaaattgaa 5580
gtaaaaaaaa aaaaaaaaaa 5600
<210> SEQ ID NO 9
<211> LENGTH: 5477
<212> TYPE: DNA
<213> ORGANISM: Homo sapiens
<400> SEQUENCE: 9
aaaaagagaa actgttggga gaggaatcgt atctccatat ttcttctttc agccccaatc 60
caagggttgt agctggaact ttccatcagt tcttcctttc tttttcctct ctaagccttt 120
gccttgctct gtcacagtga agtcagccag agcagggctg ttaaactctg tgaaatttgt 180
cataagggtg tcaggtattt cttactggct tccaaagaaa catagataaa gaaatctttc 240
ctgtggcttc ccttggcagg ctgcattcag aaggtctctc agttgaagaa agagcttgga 300
ggacaacagc acaacaggag agtaaaagat gccccagggc tgaggcctcc gctcaggcag 360
ccgcatctgg ggtcaatcat actcaccttg cccgggccat gctccagcaa aatcaagctg 420
ttttcttttg aaagttcaaa ctcatcaaga ttatgctgct cactcttatc attctgttgc 480
cagtagtttc aaaatttagt tttgttagtc tctcagcacc gcagcactgg agctgtcctg 540
aaggtactct cgcaggaaat gggaattcta cttgtgtggg tcctgcaccc ttcttaattt 600
tctcccatgg aaatagtatc tttaggattg acacagaagg aaccaattat gagcaattgg 660
tggtggatgc tggtgtctca gtgatcatgg attttcatta taatgagaaa agaatctatt 720
gggtggattt agaaagacaa cttttgcaaa gagtttttct gaatgggtca aggcaagaga 780
gagtatgtaa tatagagaaa aatgtttctg gaatggcaat aaattggata aatgaagaag 840
ttatttggtc aaatcaacag gaaggaatca ttacagtaac agatatgaaa ggaaataatt 900
cccacattct tttaagtgct ttaaaatatc ctgcaaatgt agcagttgat ccagtagaaa 960
ggtttatatt ttggtcttca gaggtggctg gaagccttta tagagcagat ctcgatggtg 1020
tgggagtgaa ggctctgttg gagacatcag agaaaataac agctgtgtca ttggatgtgc 1080
ttgataagcg gctgttttgg attcagtaca acagagaagg aagcaattct cttatttgct 1140
cctgtgatta tgatggaggt tctgtccaca ttagtaaaca tccaacacag cataatttgt 1200
ttgcaatgtc cctttttggt gaccgtatct tctattcaac atggaaaatg aagacaattt 1260
ggatagccaa caaacacact ggaaaggaca tggttagaat taacctccat tcatcatttg 1320
taccacttgg tgaactgaaa gtagtgcatc cacttgcaca acccaaggca gaagatgaca 1380
cttgggagcc tgagcagaaa ctttgcaaat tgaggaaagg aaactgcagc agcactgtgt 1440
gtgggcaaga cctccagtca cacttgtgca tgtgtgcaga gggatacgcc ctaagtcgag 1500
accggaagta ctgtgaagat gttaatgaat gtgctttttg gaatcatggc tgtactcttg 1560
ggtgtaaaaa cacccctgga tcctattact gcacgtgccc tgtaggattt gttctgcttc 1620
ctgatgggaa acgatgtcat caacttgttt cctgtccacg caatgtgtct gaatgcagcc 1680
atgactgtgt tctgacatca gaaggtccct tatgtttctg tcctgaaggc tcagtgcttg 1740
agagagatgg gaaaacatgt agcggttgtt cctcacccga taatggtgga tgtagccagc 1800
tctgcgttcc tcttagccca gtatcctggg aatgtgattg ctttcctggg tatgacctac 1860
aactggatga aaaaagctgt gcagcttcag gaccacaacc atttttgctg tttgccaatt 1920
ctcaagatat tcgacacatg cattttgatg gaacagacta tggaactctg ctcagccagc 1980
agatgggaat ggtttatgcc ctagatcatg accctgtgga aaataagata tactttgccc 2040
atacagccct gaagtggata gagagagcta atatggatgg ttcccagcga gaaaggctta 2100
ttgaggaagg agtagatgtg ccagaaggtc ttgctgtgga ctggattggc cgtagattct 2160
attggacaga cagagggaaa tctctgattg gaaggagtga tttaaatggg aaacgttcca 2220
aaataatcac taaggagaac atctctcaac cacgaggaat tgctgttcat ccaatggcca 2280
agagattatt ctggactgat acagggatta atccacgaat tgaaagttct tccctccaag 2340
gccttggccg tctggttata gccagctctg atctaatctg gcccagtgga ataacgattg 2400
acttcttaac tgacaagttg tactggtgcg atgccaagca gtctgtgatt gaaatggcca 2460
atctggatgg ttcaaaacgc cgaagactta cccagaatga tgtaggtcac ccatttgctg 2520
tagcagtgtt tgaggattat gtgtggttct cagattgggc tatgccatca gtaatgagag 2580
taaacaagag gactggcaaa gatagagtac gtctccaagg cagcatgctg aagccctcat 2640
cactggttgt ggttcatcca ttggcaaaac caggagcaga tccctgctta tatcaaaacg 2700
gaggctgtga acatatttgc aaaaagaggc ttggaactgc ttggtgttcg tgtcgtgaag 2760
gttttatgaa agcctcagat gggaaaacgt gtctggctct ggatggtcat cagctgttgg 2820
caggtggtga agttgatcta aagaaccaag taacaccatt ggacatcttg tccaagacta 2880
gagtgtcaga agataacatt acagaatctc aacacatgct agtggctgaa atcatggtgt 2940
cagatcaaga tgactgtgct cctgtgggat gcagcatgta tgctcggtgt atttcagagg 3000
gagaggatgc cacatgtcag tgtttgaaag gatttgctgg ggatggaaaa ctatgttctg 3060
atatagatga atgtgagatg ggtgtcccag tgtgcccccc tgcctcctcc aagtgcatca 3120
acaccgaagg tggttatgtc tgccggtgct cagaaggcta ccaaggagat gggattcact 3180
gtcttgactc tactccaccc cctcacctca gggaagatga ccaccactat tccgtaagaa 3240
atagtgactc tgaatgtccc ctgtcccacg atgggtactg cctccatgat ggtgtgtgca 3300
tgtatattga agcattggac aagtatgcat gcaactgtgt tgttggctac atcggggagc 3360
gatgtcagta ccgagacctg aagtggtggg aactgcgcca cgctggccac gggcagcagc 3420
agaaggtcat cgtggtggct gtctgcgtgg tggtgcttgt catgctgctc ctcctgagcc 3480
tgtggggggc ccactactac aggactcaga agctgctatc gaaaaaccca aagaatcctt 3540
atgaggagtc gagcagagat gtgaggagtc gcaggcctgc tgacactgag gatgggatgt 3600
cctcttgccc tcaaccttgg tttgtggtta taaaagaaca ccaagacctc aagaatgggg 3660
gtcaaccagt ggctggtgag gatggccagg cagcagatgg gtcaatgcaa ccaacttcat 3720
ggaggcagga gccccagtta tgtggaatgg gcacagagca aggctgctgg attccagtat 3780
ccagtgataa gggctcctgt ccccaggtaa tggagcgaag ctttcatatg ccctcctatg 3840
ggacacagac ccttgaaggg ggtgtcgaga agccccattc tctcctatca gctaacccat 3900
tatggcaaca aagggccctg gacccaccac accaaatgga gctgactcag tgaaaactgg 3960
aattaaaagg aaagtcaaga agaatgaact atgtcgatgc acagtatctt ttctttcaaa 4020
agtagagcaa aactataggt tttggttcca caatctctac gactaatcac ctactcaatg 4080
cctggagaca gatacgtagt tgtgcttttg tttgctcttt taagcagtct cactgcagtc 4140
ttatttccaa gtaagagtac tgggagaatc actaggtaac ttattagaaa cccaaattgg 4200
gacaacagtg ctttgtaaat tgtgttgtct tcagcagtca atacaaatag atttttgttt 4260
ttgttgttcc tgcagcccca gaagaaatta ggggttaaag cagacagtca cactggtttg 4320
gtcagttaca aagtaatttc tttgatctgg acagaacatt tatatcagtt tcatgaaatg 4380
attggaatat tacaataccg ttaagataca gtgtaggcat ttaactcctc attggcgtgg 4440
tccatgctga tgattttgca aaatgagttg tgatgaatca atgaaaaatg taatttagaa 4500
actgatttct tcagaattag atggcttatt ttttaaaata tttgaatgaa aacattttat 4560
ttttaaaata ttacacagga ggcttcggag tttcttagtc attactgtcc ttttccccta 4620
cagaattttc cctcttggtg tgattgcaca gaatttgtat gtattttcag ttacaagatt 4680
gtaagtaaat tgcctgattt gttttcatta tagacaacga tgaatttctt ctaattattt 4740
aaataaaatc accaaaaaca taaacatttt attgtatgcc tgattaagta gttaattata 4800
gtctaaggca gtactagagt tgaaccaaaa tgatttgtca agcttgctga tgtttctgtt 4860
tttcgttttt tttttttttc cggagagagg ataggatctc actctgttat ccaggctgga 4920
gtgtgcaatg gcacaatcat agctcagtgc agcctcaaac tcctgggctc aagcaatcct 4980
cctgcctcag cctcccgagt aactaggacc acaggcacag gccaccatgc ctggctaagg 5040
tttttatttt tattttttgt agacatgggg atcacacaat gttgcccagg ctggtcttga 5100
actcctggcc tcaagcaagg tcgtgctggt aattttgcaa aatgaattgt gattgacttt 5160
cagcctccca acgtattaga ttataggcat tagccatggt gcccagcctt gtaactttta 5220
aaaaaatttt ttaatctaca actctgtaga ttaaaatttc acatggtgtt ctaattaaat 5280
atttttcttg cagccaagat attgttacta cagataacac aacctgatat ggtaacttta 5340
aattttgggg gctttgaatc attcagttta tgcattaact agtccctttg tttatctttc 5400
atttctcaac cccttgtact ttggtgatac cagacatcag aataaaaaga aattgaagta 5460
aaaaaaaaaa aaaaaaa 5477
<210> SEQ ID NO 10
<211> LENGTH: 5474
<212> TYPE: DNA
<213> ORGANISM: Homo sapiens
<400> SEQUENCE: 10
aaaaagagaa actgttggga gaggaatcgt atctccatat ttcttctttc agccccaatc 60
caagggttgt agctggaact ttccatcagt tcttcctttc tttttcctct ctaagccttt 120
gccttgctct gtcacagtga agtcagccag agcagggctg ttaaactctg tgaaatttgt 180
cataagggtg tcaggtattt cttactggct tccaaagaaa catagataaa gaaatctttc 240
ctgtggcttc ccttggcagg ctgcattcag aaggtctctc agttgaagaa agagcttgga 300
ggacaacagc acaacaggag agtaaaagat gccccagggc tgaggcctcc gctcaggcag 360
ccgcatctgg ggtcaatcat actcaccttg cccgggccat gctccagcaa aatcaagctg 420
ttttcttttg aaagttcaaa ctcatcaaga ttatgctgct cactcttatc attctgttgc 480
cagtagtttc aaaatttagt tttgttagtc tctcagcacc gcagcactgg agctgtcctg 540
aaggtactct cgcaggaaat gggaattcta cttgtgtggg tcctgcaccc ttcttaattt 600
tctcccatgg aaatagtatc tttaggattg acacagaagg aaccaattat gagcaattgg 660
tggtggatgc tggtgtctca gtgatcatgg attttcatta taatgagaaa agaatctatt 720
gggtggattt agaaagacaa cttttgcaaa gagtttttct gaatgggtca aggcaagaga 780
gagtatgtaa tatagagaaa aatgtttctg gaatggcaat aaattggata aatgaagaag 840
ttatttggtc aaatcaacag gaaggaatca ttacagtaac agatatgaaa ggaaataatt 900
cccacattct tttaagtgct ttaaaatatc ctgcaaatgt agcagttgat ccagtagaaa 960
ggtttatatt ttggtcttca gaggtggctg gaagccttta tagagcagat ctcgatggtg 1020
tgggagtgaa ggctctgttg gagacatcag agaaaataac agctgtgtca ttggatgtgc 1080
ttgataagcg gctgttttgg attcagtaca acagagaagg aagcaattct cttatttgct 1140
cctgtgatta tgatggaggt tctgtccaca ttagtaaaca tccaacacag cataatttgt 1200
ttgcaatgtc cctttttggt gaccgtatct tctattcaac atggaaaatg aagacaattt 1260
ggatagccaa caaacacact ggaaaggaca tggttagaat taacctccat tcatcatttg 1320
taccacttgg tgaactgaaa gtagtgcatc cacttgcaca acccaaggca gaagatgaca 1380
cttgggagcc tgatgttaat gaatgtgctt tttggaatca tggctgtact cttgggtgta 1440
aaaacacccc tggatcctat tactgcacgt gccctgtagg atttgttctg cttcctgatg 1500
ggaaacgatg tcatcaactt gtttcctgtc cacgcaatgt gtctgaatgc agccatgact 1560
gtgttctgac atcagaaggt cccttatgtt tctgtcctga aggctcagtg cttgagagag 1620
atgggaaaac atgtagcggt tgttcctcac ccgataatgg tggatgtagc cagctctgcg 1680
ttcctcttag cccagtatcc tgggaatgtg attgctttcc tgggtatgac ctacaactgg 1740
atgaaaaaag ctgtgcagct tcaggaccac aaccattttt gctgtttgcc aattctcaag 1800
atattcgaca catgcatttt gatggaacag actatggaac tctgctcagc cagcagatgg 1860
gaatggttta tgccctagat catgaccctg tggaaaataa gatatacttt gcccatacag 1920
ccctgaagtg gatagagaga gctaatatgg atggttccca gcgagaaagg cttattgagg 1980
aaggagtaga tgtgccagaa ggtcttgctg tggactggat tggccgtaga ttctattgga 2040
cagacagagg gaaatctctg attggaagga gtgatttaaa tgggaaacgt tccaaaataa 2100
tcactaagga gaacatctct caaccacgag gaattgctgt tcatccaatg gccaagagat 2160
tattctggac tgatacaggg attaatccac gaattgaaag ttcttccctc caaggccttg 2220
gccgtctggt tatagccagc tctgatctaa tctggcccag tggaataacg attgacttct 2280
taactgacaa gttgtactgg tgcgatgcca agcagtctgt gattgaaatg gccaatctgg 2340
atggttcaaa acgccgaaga cttacccaga atgatgtagg tcacccattt gctgtagcag 2400
tgtttgagga ttatgtgtgg ttctcagatt gggctatgcc atcagtaatg agagtaaaca 2460
agaggactgg caaagataga gtacgtctcc aaggcagcat gctgaagccc tcatcactgg 2520
ttgtggttca tccattggca aaaccaggag cagatccctg cttatatcaa aacggaggct 2580
gtgaacatat ttgcaaaaag aggcttggaa ctgcttggtg ttcgtgtcgt gaaggtttta 2640
tgaaagcctc agatgggaaa acgtgtctgg ctctggatgg tcatcagctg ttggcaggtg 2700
gtgaagttga tctaaagaac caagtaacac cattggacat cttgtccaag actagagtgt 2760
cagaagataa cattacagaa tctcaacaca tgctagtggc tgaaatcatg gtgtcagatc 2820
aagatgactg tgctcctgtg ggatgcagca tgtatgctcg gtgtatttca gagggagagg 2880
atgccacatg tcagtgtttg aaaggatttg ctggggatgg aaaactatgt tctgatatag 2940
atgaatgtga gatgggtgtc ccagtgtgcc cccctgcctc ctccaagtgc atcaacaccg 3000
aaggtggtta tgtctgccgg tgctcagaag gctaccaagg agatgggatt cactgtcttg 3060
atattgatga gtgccaactg ggggagcaca gctgtggaga gaatgccagc tgcacaaata 3120
cagagggagg ctatacctgc atgtgtgctg gacgcctgtc tgaaccagga ctgatttgcc 3180
ctgactctac tccaccccct cacctcaggg aagatgacca ccactattcc gtaagaaata 3240
gtgactctga atgtcccctg tcccacgatg ggtactgcct ccatgatggt gtgtgcatgt 3300
atattgaagc attggacaag tatgcatgca actgtgttgt tggctacatc ggggagcgat 3360
gtcagtaccg agacctgaag tggtgggaac tgcgccacgc tggccacggg cagcagcaga 3420
aggtcatcgt ggtggctgtc tgcgtggtgg tgcttgtcat gctgctcctc ctgagcctgt 3480
ggggggccca ctactacagg actcagaagc tgctatcgaa aaacccaaag aatccttatg 3540
aggagtcgag cagagatgtg aggagtcgca ggcctgctga cactgaggat gggatgtcct 3600
cttgccctca accttggttt gtggttataa aagaacacca agacctcaag aatgggggtc 3660
aaccagtggc tggtgaggat ggccaggcag cagatgggtc aatgcaacca acttcatgga 3720
ggcaggagcc ccagttatgt ggaatgggca cagagcaagg ctgctggatt ccagtatcca 3780
gtgataaggg ctcctgtccc caggtaatgg agcgaagctt tcatatgccc tcctatggga 3840
cacagaccct tgaagggggt gtcgagaagc cccattctct cctatcagct aacccattat 3900
ggcaacaaag ggccctggac ccaccacacc aaatggagct gactcagtga aaactggaat 3960
taaaaggaaa gtcaagaaga atgaactatg tcgatgcaca gtatcttttc tttcaaaagt 4020
agagcaaaac tataggtttt ggttccacaa tctctacgac taatcaccta ctcaatgcct 4080
ggagacagat acgtagttgt gcttttgttt gctcttttaa gcagtctcac tgcagtctta 4140
tttccaagta agagtactgg gagaatcact aggtaactta ttagaaaccc aaattgggac 4200
aacagtgctt tgtaaattgt gttgtcttca gcagtcaata caaatagatt tttgtttttg 4260
ttgttcctgc agccccagaa gaaattaggg gttaaagcag acagtcacac tggtttggtc 4320
agttacaaag taatttcttt gatctggaca gaacatttat atcagtttca tgaaatgatt 4380
ggaatattac aataccgtta agatacagtg taggcattta actcctcatt ggcgtggtcc 4440
atgctgatga ttttgcaaaa tgagttgtga tgaatcaatg aaaaatgtaa tttagaaact 4500
gatttcttca gaattagatg gcttattttt taaaatattt gaatgaaaac attttatttt 4560
taaaatatta cacaggaggc ttcggagttt cttagtcatt actgtccttt tcccctacag 4620
aattttccct cttggtgtga ttgcacagaa tttgtatgta ttttcagtta caagattgta 4680
agtaaattgc ctgatttgtt ttcattatag acaacgatga atttcttcta attatttaaa 4740
taaaatcacc aaaaacataa acattttatt gtatgcctga ttaagtagtt aattatagtc 4800
taaggcagta ctagagttga accaaaatga tttgtcaagc ttgctgatgt ttctgttttt 4860
cgtttttttt ttttttccgg agagaggata ggatctcact ctgttatcca ggctggagtg 4920
tgcaatggca caatcatagc tcagtgcagc ctcaaactcc tgggctcaag caatcctcct 4980
gcctcagcct cccgagtaac taggaccaca ggcacaggcc accatgcctg gctaaggttt 5040
ttatttttat tttttgtaga catggggatc acacaatgtt gcccaggctg gtcttgaact 5100
cctggcctca agcaaggtcg tgctggtaat tttgcaaaat gaattgtgat tgactttcag 5160
cctcccaacg tattagatta taggcattag ccatggtgcc cagccttgta acttttaaaa 5220
aaatttttta atctacaact ctgtagatta aaatttcaca tggtgttcta attaaatatt 5280
tttcttgcag ccaagatatt gttactacag ataacacaac ctgatatggt aactttaaat 5340
tttgggggct ttgaatcatt cagtttatgc attaactagt ccctttgttt atctttcatt 5400
tctcaacccc ttgtactttg gtgataccag acatcagaat aaaaagaaat tgaagtaaaa 5460
aaaaaaaaaa aaaa 5474
<210> SEQ ID NO 11
<211> LENGTH: 3677
<212> TYPE: DNA
<213> ORGANISM: Homo sapiens
<400> SEQUENCE: 11
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa aaaatcagtt cgaggaaagg gaaaggggca aaaacgaaag 1500
cgcaagaaat cccggtataa gtcctggagc gtgtacgttg gtgcccgctg ctgtctaatg 1560
ccctggagcc tccctggccc ccatccctgt gggccttgct cagagcggag aaagcatttg 1620
tttgtacaag atccgcagac gtgtaaatgt tcctgcaaaa acacagactc gcgttgcaag 1680
gcgaggcagc ttgagttaaa cgaacgtact tgcagatgtg acaagccgag gcggtgagcc 1740
gggcaggagg aaggagcctc cctcagggtt tcgggaacca gatctctcac caggaaagac 1800
tgatacagaa cgatcgatac agaaaccacg ctgccgccac cacaccatca ccatcgacag 1860
aacagtcctt aatccagaaa cctgaaatga aggaagagga gactctgcgc agagcacttt 1920
gggtccggag ggcgagactc cggcggaagc attcccgggc gggtgaccca gcacggtccc 1980
tcttggaatt ggattcgcca ttttattttt cttgctgcta aatcaccgag cccggaagat 2040
tagagagttt tatttctggg attcctgtag acacacccac ccacatacat acatttatat 2100
atatatatat tatatatata taaaaataaa tatctctatt ttatatatat aaaatatata 2160
tattcttttt ttaaattaac agtgctaatg ttattggtgt cttcactgga tgtatttgac 2220
tgctgtggac ttgagttggg aggggaatgt tcccactcag atcctgacag ggaagaggag 2280
gagatgagag actctggcat gatctttttt ttgtcccact tggtggggcc agggtcctct 2340
cccctgccca ggaatgtgca aggccagggc atgggggcaa atatgaccca gttttgggaa 2400
caccgacaaa cccagccctg gcgctgagcc tctctacccc aggtcagacg gacagaaaga 2460
cagatcacag gtacagggat gaggacaccg gctctgacca ggagtttggg gagcttcagg 2520
acattgctgt gctttgggga ttccctccac atgctgcacg cgcatctcgc ccccaggggc 2580
actgcctgga agattcagga gcctgggcgg ccttcgctta ctctcacctg cttctgagtt 2640
gcccaggaga ccactggcag atgtcccggc gaagagaaga gacacattgt tggaagaagc 2700
agcccatgac agctcccctt cctgggactc gccctcatcc tcttcctgct ccccttcctg 2760
gggtgcagcc taaaaggacc tatgtcctca caccattgaa accactagtt ctgtcccccc 2820
aggagacctg gttgtgtgtg tgtgagtggt tgaccttcct ccatcccctg gtccttccct 2880
tcccttcccg aggcacagag agacagggca ggatccacgt gcccattgtg gaggcagaga 2940
aaagagaaag tgttttatat acggtactta tttaatatcc ctttttaatt agaaattaaa 3000
acagttaatt taattaaaga gtagggtttt ttttcagtat tcttggttaa tatttaattt 3060
caactattta tgagatgtat cttttgctct ctcttgctct cttatttgta ccggtttttg 3120
tatataaaat tcatgtttcc aatctctctc tccctgatcg gtgacagtca ctagcttatc 3180
ttgaacagat atttaatttt gctaacactc agctctgccc tccccgatcc cctggctccc 3240
cagcacacat tcctttgaaa taaggtttca atatacatct acatactata tatatatttg 3300
gcaacttgta tttgtgtgta tatatatata tatatgttta tgtatatatg tgattctgat 3360
aaaatagaca ttgctattct gttttttata tgtaaaaaca aaacaagaaa aaatagagaa 3420
ttctacatac taaatctctc tcctttttta attttaatat ttgttatcat ttatttattg 3480
gtgctactgt ttatccgtaa taattgtggg gaaaagatat taacatcacg tctttgtctc 3540
tagtgcagtt tttcgagata ttccgtagta catatttatt tttaaacaac gacaaagaaa 3600
tacagatata tcttaaaaaa aaaaaagcat tttgtattaa agaatttaat tctgatctca 3660
aaaaaaaaaa aaaaaaa 3677
<210> SEQ ID NO 12
<211> LENGTH: 3677
<212> TYPE: DNA
<213> ORGANISM: Homo sapiens
<400> SEQUENCE: 12
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa aaaatcagtt cgaggaaagg gaaaggggca aaaacgaaag 1500
cgcaagaaat cccggtataa gtcctggagc gtgtacgttg gtgcccgctg ctgtctaatg 1560
ccctggagcc tccctggccc ccatccctgt gggccttgct cagagcggag aaagcatttg 1620
tttgtacaag atccgcagac gtgtaaatgt tcctgcaaaa acacagactc gcgttgcaag 1680
gcgaggcagc ttgagttaaa cgaacgtact tgcagatgtg acaagccgag gcggtgagcc 1740
gggcaggagg aaggagcctc cctcagggtt tcgggaacca gatctctcac caggaaagac 1800
tgatacagaa cgatcgatac agaaaccacg ctgccgccac cacaccatca ccatcgacag 1860
aacagtcctt aatccagaaa cctgaaatga aggaagagga gactctgcgc agagcacttt 1920
gggtccggag ggcgagactc cggcggaagc attcccgggc gggtgaccca gcacggtccc 1980
tcttggaatt ggattcgcca ttttattttt cttgctgcta aatcaccgag cccggaagat 2040
tagagagttt tatttctggg attcctgtag acacacccac ccacatacat acatttatat 2100
atatatatat tatatatata taaaaataaa tatctctatt ttatatatat aaaatatata 2160
tattcttttt ttaaattaac agtgctaatg ttattggtgt cttcactgga tgtatttgac 2220
tgctgtggac ttgagttggg aggggaatgt tcccactcag atcctgacag ggaagaggag 2280
gagatgagag actctggcat gatctttttt ttgtcccact tggtggggcc agggtcctct 2340
cccctgccca ggaatgtgca aggccagggc atgggggcaa atatgaccca gttttgggaa 2400
caccgacaaa cccagccctg gcgctgagcc tctctacccc aggtcagacg gacagaaaga 2460
cagatcacag gtacagggat gaggacaccg gctctgacca ggagtttggg gagcttcagg 2520
acattgctgt gctttgggga ttccctccac atgctgcacg cgcatctcgc ccccaggggc 2580
actgcctgga agattcagga gcctgggcgg ccttcgctta ctctcacctg cttctgagtt 2640
gcccaggaga ccactggcag atgtcccggc gaagagaaga gacacattgt tggaagaagc 2700
agcccatgac agctcccctt cctgggactc gccctcatcc tcttcctgct ccccttcctg 2760
gggtgcagcc taaaaggacc tatgtcctca caccattgaa accactagtt ctgtcccccc 2820
aggagacctg gttgtgtgtg tgtgagtggt tgaccttcct ccatcccctg gtccttccct 2880
tcccttcccg aggcacagag agacagggca ggatccacgt gcccattgtg gaggcagaga 2940
aaagagaaag tgttttatat acggtactta tttaatatcc ctttttaatt agaaattaaa 3000
acagttaatt taattaaaga gtagggtttt ttttcagtat tcttggttaa tatttaattt 3060
caactattta tgagatgtat cttttgctct ctcttgctct cttatttgta ccggtttttg 3120
tatataaaat tcatgtttcc aatctctctc tccctgatcg gtgacagtca ctagcttatc 3180
ttgaacagat atttaatttt gctaacactc agctctgccc tccccgatcc cctggctccc 3240
cagcacacat tcctttgaaa taaggtttca atatacatct acatactata tatatatttg 3300
gcaacttgta tttgtgtgta tatatatata tatatgttta tgtatatatg tgattctgat 3360
aaaatagaca ttgctattct gttttttata tgtaaaaaca aaacaagaaa aaatagagaa 3420
ttctacatac taaatctctc tcctttttta attttaatat ttgttatcat ttatttattg 3480
gtgctactgt ttatccgtaa taattgtggg gaaaagatat taacatcacg tctttgtctc 3540
tagtgcagtt tttcgagata ttccgtagta catatttatt tttaaacaac gacaaagaaa 3600
tacagatata tcttaaaaaa aaaaaagcat tttgtattaa agaatttaat tctgatctca 3660
aaaaaaaaaa aaaaaaa 3677
<210> SEQ ID NO 13
<211> LENGTH: 3626
<212> TYPE: DNA
<213> ORGANISM: Homo sapiens
<400> SEQUENCE: 13
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa aaaatcagtt cgaggaaagg gaaaggggca aaaacgaaag 1500
cgcaagaaat cccggtataa gtcctggagc gttccctgtg ggccttgctc agagcggaga 1560
aagcatttgt ttgtacaaga tccgcagacg tgtaaatgtt cctgcaaaaa cacagactcg 1620
cgttgcaagg cgaggcagct tgagttaaac gaacgtactt gcagatgtga caagccgagg 1680
cggtgagccg ggcaggagga aggagcctcc ctcagggttt cgggaaccag atctctcacc 1740
aggaaagact gatacagaac gatcgataca gaaaccacgc tgccgccacc acaccatcac 1800
catcgacaga acagtcctta atccagaaac ctgaaatgaa ggaagaggag actctgcgca 1860
gagcactttg ggtccggagg gcgagactcc ggcggaagca ttcccgggcg ggtgacccag 1920
cacggtccct cttggaattg gattcgccat tttatttttc ttgctgctaa atcaccgagc 1980
ccggaagatt agagagtttt atttctggga ttcctgtaga cacacccacc cacatacata 2040
catttatata tatatatatt atatatatat aaaaataaat atctctattt tatatatata 2100
aaatatatat attctttttt taaattaaca gtgctaatgt tattggtgtc ttcactggat 2160
gtatttgact gctgtggact tgagttggga ggggaatgtt cccactcaga tcctgacagg 2220
gaagaggagg agatgagaga ctctggcatg atcttttttt tgtcccactt ggtggggcca 2280
gggtcctctc ccctgcccag gaatgtgcaa ggccagggca tgggggcaaa tatgacccag 2340
ttttgggaac accgacaaac ccagccctgg cgctgagcct ctctacccca ggtcagacgg 2400
acagaaagac agatcacagg tacagggatg aggacaccgg ctctgaccag gagtttgggg 2460
agcttcagga cattgctgtg ctttggggat tccctccaca tgctgcacgc gcatctcgcc 2520
cccaggggca ctgcctggaa gattcaggag cctgggcggc cttcgcttac tctcacctgc 2580
ttctgagttg cccaggagac cactggcaga tgtcccggcg aagagaagag acacattgtt 2640
ggaagaagca gcccatgaca gctccccttc ctgggactcg ccctcatcct cttcctgctc 2700
cccttcctgg ggtgcagcct aaaaggacct atgtcctcac accattgaaa ccactagttc 2760
tgtcccccca ggagacctgg ttgtgtgtgt gtgagtggtt gaccttcctc catcccctgg 2820
tccttccctt cccttcccga ggcacagaga gacagggcag gatccacgtg cccattgtgg 2880
aggcagagaa aagagaaagt gttttatata cggtacttat ttaatatccc tttttaatta 2940
gaaattaaaa cagttaattt aattaaagag tagggttttt tttcagtatt cttggttaat 3000
atttaatttc aactatttat gagatgtatc ttttgctctc tcttgctctc ttatttgtac 3060
cggtttttgt atataaaatt catgtttcca atctctctct ccctgatcgg tgacagtcac 3120
tagcttatct tgaacagata tttaattttg ctaacactca gctctgccct ccccgatccc 3180
ctggctcccc agcacacatt cctttgaaat aaggtttcaa tatacatcta catactatat 3240
atatatttgg caacttgtat ttgtgtgtat atatatatat atatgtttat gtatatatgt 3300
gattctgata aaatagacat tgctattctg ttttttatat gtaaaaacaa aacaagaaaa 3360
aatagagaat tctacatact aaatctctct ccttttttaa ttttaatatt tgttatcatt 3420
tatttattgg tgctactgtt tatccgtaat aattgtgggg aaaagatatt aacatcacgt 3480
ctttgtctct agtgcagttt ttcgagatat tccgtagtac atatttattt ttaaacaacg 3540
acaaagaaat acagatatat cttaaaaaaa aaaaagcatt ttgtattaaa gaatttaatt 3600
ctgatctcaa aaaaaaaaaa aaaaaa 3626
<210> SEQ ID NO 14
<211> LENGTH: 3626
<212> TYPE: DNA
<213> ORGANISM: Homo sapiens
<400> SEQUENCE: 14
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa aaaatcagtt cgaggaaagg gaaaggggca aaaacgaaag 1500
cgcaagaaat cccggtataa gtcctggagc gttccctgtg ggccttgctc agagcggaga 1560
aagcatttgt ttgtacaaga tccgcagacg tgtaaatgtt cctgcaaaaa cacagactcg 1620
cgttgcaagg cgaggcagct tgagttaaac gaacgtactt gcagatgtga caagccgagg 1680
cggtgagccg ggcaggagga aggagcctcc ctcagggttt cgggaaccag atctctcacc 1740
aggaaagact gatacagaac gatcgataca gaaaccacgc tgccgccacc acaccatcac 1800
catcgacaga acagtcctta atccagaaac ctgaaatgaa ggaagaggag actctgcgca 1860
gagcactttg ggtccggagg gcgagactcc ggcggaagca ttcccgggcg ggtgacccag 1920
cacggtccct cttggaattg gattcgccat tttatttttc ttgctgctaa atcaccgagc 1980
ccggaagatt agagagtttt atttctggga ttcctgtaga cacacccacc cacatacata 2040
catttatata tatatatatt atatatatat aaaaataaat atctctattt tatatatata 2100
aaatatatat attctttttt taaattaaca gtgctaatgt tattggtgtc ttcactggat 2160
gtatttgact gctgtggact tgagttggga ggggaatgtt cccactcaga tcctgacagg 2220
gaagaggagg agatgagaga ctctggcatg atcttttttt tgtcccactt ggtggggcca 2280
gggtcctctc ccctgcccag gaatgtgcaa ggccagggca tgggggcaaa tatgacccag 2340
ttttgggaac accgacaaac ccagccctgg cgctgagcct ctctacccca ggtcagacgg 2400
acagaaagac agatcacagg tacagggatg aggacaccgg ctctgaccag gagtttgggg 2460
agcttcagga cattgctgtg ctttggggat tccctccaca tgctgcacgc gcatctcgcc 2520
cccaggggca ctgcctggaa gattcaggag cctgggcggc cttcgcttac tctcacctgc 2580
ttctgagttg cccaggagac cactggcaga tgtcccggcg aagagaagag acacattgtt 2640
ggaagaagca gcccatgaca gctccccttc ctgggactcg ccctcatcct cttcctgctc 2700
cccttcctgg ggtgcagcct aaaaggacct atgtcctcac accattgaaa ccactagttc 2760
tgtcccccca ggagacctgg ttgtgtgtgt gtgagtggtt gaccttcctc catcccctgg 2820
tccttccctt cccttcccga ggcacagaga gacagggcag gatccacgtg cccattgtgg 2880
aggcagagaa aagagaaagt gttttatata cggtacttat ttaatatccc tttttaatta 2940
gaaattaaaa cagttaattt aattaaagag tagggttttt tttcagtatt cttggttaat 3000
atttaatttc aactatttat gagatgtatc ttttgctctc tcttgctctc ttatttgtac 3060
cggtttttgt atataaaatt catgtttcca atctctctct ccctgatcgg tgacagtcac 3120
tagcttatct tgaacagata tttaattttg ctaacactca gctctgccct ccccgatccc 3180
ctggctcccc agcacacatt cctttgaaat aaggtttcaa tatacatcta catactatat 3240
atatatttgg caacttgtat ttgtgtgtat atatatatat atatgtttat gtatatatgt 3300
gattctgata aaatagacat tgctattctg ttttttatat gtaaaaacaa aacaagaaaa 3360
aatagagaat tctacatact aaatctctct ccttttttaa ttttaatatt tgttatcatt 3420
tatttattgg tgctactgtt tatccgtaat aattgtgggg aaaagatatt aacatcacgt 3480
ctttgtctct agtgcagttt ttcgagatat tccgtagtac atatttattt ttaaacaacg 3540
acaaagaaat acagatatat cttaaaaaaa aaaaagcatt ttgtattaaa gaatttaatt 3600
ctgatctcaa aaaaaaaaaa aaaaaa 3626
<210> SEQ ID NO 15
<211> LENGTH: 3608
<212> TYPE: DNA
<213> ORGANISM: Homo sapiens
<400> SEQUENCE: 15
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa aaaatcagtt cgaggaaagg gaaaggggca aaaacgaaag 1500
cgcaagaaat cccgtccctg tgggccttgc tcagagcgga gaaagcattt gtttgtacaa 1560
gatccgcaga cgtgtaaatg ttcctgcaaa aacacagact cgcgttgcaa ggcgaggcag 1620
cttgagttaa acgaacgtac ttgcagatgt gacaagccga ggcggtgagc cgggcaggag 1680
gaaggagcct ccctcagggt ttcgggaacc agatctctca ccaggaaaga ctgatacaga 1740
acgatcgata cagaaaccac gctgccgcca ccacaccatc accatcgaca gaacagtcct 1800
taatccagaa acctgaaatg aaggaagagg agactctgcg cagagcactt tgggtccgga 1860
gggcgagact ccggcggaag cattcccggg cgggtgaccc agcacggtcc ctcttggaat 1920
tggattcgcc attttatttt tcttgctgct aaatcaccga gcccggaaga ttagagagtt 1980
ttatttctgg gattcctgta gacacaccca cccacataca tacatttata tatatatata 2040
ttatatatat ataaaaataa atatctctat tttatatata taaaatatat atattctttt 2100
tttaaattaa cagtgctaat gttattggtg tcttcactgg atgtatttga ctgctgtgga 2160
cttgagttgg gaggggaatg ttcccactca gatcctgaca gggaagagga ggagatgaga 2220
gactctggca tgatcttttt tttgtcccac ttggtggggc cagggtcctc tcccctgccc 2280
aggaatgtgc aaggccaggg catgggggca aatatgaccc agttttggga acaccgacaa 2340
acccagccct ggcgctgagc ctctctaccc caggtcagac ggacagaaag acagatcaca 2400
ggtacaggga tgaggacacc ggctctgacc aggagtttgg ggagcttcag gacattgctg 2460
tgctttgggg attccctcca catgctgcac gcgcatctcg cccccagggg cactgcctgg 2520
aagattcagg agcctgggcg gccttcgctt actctcacct gcttctgagt tgcccaggag 2580
accactggca gatgtcccgg cgaagagaag agacacattg ttggaagaag cagcccatga 2640
cagctcccct tcctgggact cgccctcatc ctcttcctgc tccccttcct ggggtgcagc 2700
ctaaaaggac ctatgtcctc acaccattga aaccactagt tctgtccccc caggagacct 2760
ggttgtgtgt gtgtgagtgg ttgaccttcc tccatcccct ggtccttccc ttcccttccc 2820
gaggcacaga gagacagggc aggatccacg tgcccattgt ggaggcagag aaaagagaaa 2880
gtgttttata tacggtactt atttaatatc cctttttaat tagaaattaa aacagttaat 2940
ttaattaaag agtagggttt tttttcagta ttcttggtta atatttaatt tcaactattt 3000
atgagatgta tcttttgctc tctcttgctc tcttatttgt accggttttt gtatataaaa 3060
ttcatgtttc caatctctct ctccctgatc ggtgacagtc actagcttat cttgaacaga 3120
tatttaattt tgctaacact cagctctgcc ctccccgatc ccctggctcc ccagcacaca 3180
ttcctttgaa ataaggtttc aatatacatc tacatactat atatatattt ggcaacttgt 3240
atttgtgtgt atatatatat atatatgttt atgtatatat gtgattctga taaaatagac 3300
attgctattc tgttttttat atgtaaaaac aaaacaagaa aaaatagaga attctacata 3360
ctaaatctct ctcctttttt aattttaata tttgttatca tttatttatt ggtgctactg 3420
tttatccgta ataattgtgg ggaaaagata ttaacatcac gtctttgtct ctagtgcagt 3480
ttttcgagat attccgtagt acatatttat ttttaaacaa cgacaaagaa atacagatat 3540
atcttaaaaa aaaaaaagca ttttgtatta aagaatttaa ttctgatctc aaaaaaaaaa 3600
aaaaaaaa 3608
<210> SEQ ID NO 16
<211> LENGTH: 3608
<212> TYPE: DNA
<213> ORGANISM: Homo sapiens
<400> SEQUENCE: 16
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa aaaatcagtt cgaggaaagg gaaaggggca aaaacgaaag 1500
cgcaagaaat cccgtccctg tgggccttgc tcagagcgga gaaagcattt gtttgtacaa 1560
gatccgcaga cgtgtaaatg ttcctgcaaa aacacagact cgcgttgcaa ggcgaggcag 1620
cttgagttaa acgaacgtac ttgcagatgt gacaagccga ggcggtgagc cgggcaggag 1680
gaaggagcct ccctcagggt ttcgggaacc agatctctca ccaggaaaga ctgatacaga 1740
acgatcgata cagaaaccac gctgccgcca ccacaccatc accatcgaca gaacagtcct 1800
taatccagaa acctgaaatg aaggaagagg agactctgcg cagagcactt tgggtccgga 1860
gggcgagact ccggcggaag cattcccggg cgggtgaccc agcacggtcc ctcttggaat 1920
tggattcgcc attttatttt tcttgctgct aaatcaccga gcccggaaga ttagagagtt 1980
ttatttctgg gattcctgta gacacaccca cccacataca tacatttata tatatatata 2040
ttatatatat ataaaaataa atatctctat tttatatata taaaatatat atattctttt 2100
tttaaattaa cagtgctaat gttattggtg tcttcactgg atgtatttga ctgctgtgga 2160
cttgagttgg gaggggaatg ttcccactca gatcctgaca gggaagagga ggagatgaga 2220
gactctggca tgatcttttt tttgtcccac ttggtggggc cagggtcctc tcccctgccc 2280
aggaatgtgc aaggccaggg catgggggca aatatgaccc agttttggga acaccgacaa 2340
acccagccct ggcgctgagc ctctctaccc caggtcagac ggacagaaag acagatcaca 2400
ggtacaggga tgaggacacc ggctctgacc aggagtttgg ggagcttcag gacattgctg 2460
tgctttgggg attccctcca catgctgcac gcgcatctcg cccccagggg cactgcctgg 2520
aagattcagg agcctgggcg gccttcgctt actctcacct gcttctgagt tgcccaggag 2580
accactggca gatgtcccgg cgaagagaag agacacattg ttggaagaag cagcccatga 2640
cagctcccct tcctgggact cgccctcatc ctcttcctgc tccccttcct ggggtgcagc 2700
ctaaaaggac ctatgtcctc acaccattga aaccactagt tctgtccccc caggagacct 2760
ggttgtgtgt gtgtgagtgg ttgaccttcc tccatcccct ggtccttccc ttcccttccc 2820
gaggcacaga gagacagggc aggatccacg tgcccattgt ggaggcagag aaaagagaaa 2880
gtgttttata tacggtactt atttaatatc cctttttaat tagaaattaa aacagttaat 2940
ttaattaaag agtagggttt tttttcagta ttcttggtta atatttaatt tcaactattt 3000
atgagatgta tcttttgctc tctcttgctc tcttatttgt accggttttt gtatataaaa 3060
ttcatgtttc caatctctct ctccctgatc ggtgacagtc actagcttat cttgaacaga 3120
tatttaattt tgctaacact cagctctgcc ctccccgatc ccctggctcc ccagcacaca 3180
ttcctttgaa ataaggtttc aatatacatc tacatactat atatatattt ggcaacttgt 3240
atttgtgtgt atatatatat atatatgttt atgtatatat gtgattctga taaaatagac 3300
attgctattc tgttttttat atgtaaaaac aaaacaagaa aaaatagaga attctacata 3360
ctaaatctct ctcctttttt aattttaata tttgttatca tttatttatt ggtgctactg 3420
tttatccgta ataattgtgg ggaaaagata ttaacatcac gtctttgtct ctagtgcagt 3480
ttttcgagat attccgtagt acatatttat ttttaaacaa cgacaaagaa atacagatat 3540
atcttaaaaa aaaaaaagca ttttgtatta aagaatttaa ttctgatctc aaaaaaaaaa 3600
aaaaaaaa 3608
<210> SEQ ID NO 17
<211> LENGTH: 3554
<212> TYPE: DNA
<213> ORGANISM: Homo sapiens
<400> SEQUENCE: 17
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa tccctgtggg ccttgctcag agcggagaaa gcatttgttt 1500
gtacaagatc cgcagacgtg taaatgttcc tgcaaaaaca cagactcgcg ttgcaaggcg 1560
aggcagcttg agttaaacga acgtacttgc agatgtgaca agccgaggcg gtgagccggg 1620
caggaggaag gagcctccct cagggtttcg ggaaccagat ctctcaccag gaaagactga 1680
tacagaacga tcgatacaga aaccacgctg ccgccaccac accatcacca tcgacagaac 1740
agtccttaat ccagaaacct gaaatgaagg aagaggagac tctgcgcaga gcactttggg 1800
tccggagggc gagactccgg cggaagcatt cccgggcggg tgacccagca cggtccctct 1860
tggaattgga ttcgccattt tatttttctt gctgctaaat caccgagccc ggaagattag 1920
agagttttat ttctgggatt cctgtagaca cacccaccca catacataca tttatatata 1980
tatatattat atatatataa aaataaatat ctctatttta tatatataaa atatatatat 2040
tcttttttta aattaacagt gctaatgtta ttggtgtctt cactggatgt atttgactgc 2100
tgtggacttg agttgggagg ggaatgttcc cactcagatc ctgacaggga agaggaggag 2160
atgagagact ctggcatgat cttttttttg tcccacttgg tggggccagg gtcctctccc 2220
ctgcccagga atgtgcaagg ccagggcatg ggggcaaata tgacccagtt ttgggaacac 2280
cgacaaaccc agccctggcg ctgagcctct ctaccccagg tcagacggac agaaagacag 2340
atcacaggta cagggatgag gacaccggct ctgaccagga gtttggggag cttcaggaca 2400
ttgctgtgct ttggggattc cctccacatg ctgcacgcgc atctcgcccc caggggcact 2460
gcctggaaga ttcaggagcc tgggcggcct tcgcttactc tcacctgctt ctgagttgcc 2520
caggagacca ctggcagatg tcccggcgaa gagaagagac acattgttgg aagaagcagc 2580
ccatgacagc tccccttcct gggactcgcc ctcatcctct tcctgctccc cttcctgggg 2640
tgcagcctaa aaggacctat gtcctcacac cattgaaacc actagttctg tccccccagg 2700
agacctggtt gtgtgtgtgt gagtggttga ccttcctcca tcccctggtc cttcccttcc 2760
cttcccgagg cacagagaga cagggcagga tccacgtgcc cattgtggag gcagagaaaa 2820
gagaaagtgt tttatatacg gtacttattt aatatccctt tttaattaga aattaaaaca 2880
gttaatttaa ttaaagagta gggttttttt tcagtattct tggttaatat ttaatttcaa 2940
ctatttatga gatgtatctt ttgctctctc ttgctctctt atttgtaccg gtttttgtat 3000
ataaaattca tgtttccaat ctctctctcc ctgatcggtg acagtcacta gcttatcttg 3060
aacagatatt taattttgct aacactcagc tctgccctcc ccgatcccct ggctccccag 3120
cacacattcc tttgaaataa ggtttcaata tacatctaca tactatatat atatttggca 3180
acttgtattt gtgtgtatat atatatatat atgtttatgt atatatgtga ttctgataaa 3240
atagacattg ctattctgtt ttttatatgt aaaaacaaaa caagaaaaaa tagagaattc 3300
tacatactaa atctctctcc ttttttaatt ttaatatttg ttatcattta tttattggtg 3360
ctactgttta tccgtaataa ttgtggggaa aagatattaa catcacgtct ttgtctctag 3420
tgcagttttt cgagatattc cgtagtacat atttattttt aaacaacgac aaagaaatac 3480
agatatatct taaaaaaaaa aaagcatttt gtattaaaga atttaattct gatctcaaaa 3540
aaaaaaaaaa aaaa 3554
<210> SEQ ID NO 18
<211> LENGTH: 3554
<212> TYPE: DNA
<213> ORGANISM: Homo sapiens
<400> SEQUENCE: 18
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa tccctgtggg ccttgctcag agcggagaaa gcatttgttt 1500
gtacaagatc cgcagacgtg taaatgttcc tgcaaaaaca cagactcgcg ttgcaaggcg 1560
aggcagcttg agttaaacga acgtacttgc agatgtgaca agccgaggcg gtgagccggg 1620
caggaggaag gagcctccct cagggtttcg ggaaccagat ctctcaccag gaaagactga 1680
tacagaacga tcgatacaga aaccacgctg ccgccaccac accatcacca tcgacagaac 1740
agtccttaat ccagaaacct gaaatgaagg aagaggagac tctgcgcaga gcactttggg 1800
tccggagggc gagactccgg cggaagcatt cccgggcggg tgacccagca cggtccctct 1860
tggaattgga ttcgccattt tatttttctt gctgctaaat caccgagccc ggaagattag 1920
agagttttat ttctgggatt cctgtagaca cacccaccca catacataca tttatatata 1980
tatatattat atatatataa aaataaatat ctctatttta tatatataaa atatatatat 2040
tcttttttta aattaacagt gctaatgtta ttggtgtctt cactggatgt atttgactgc 2100
tgtggacttg agttgggagg ggaatgttcc cactcagatc ctgacaggga agaggaggag 2160
atgagagact ctggcatgat cttttttttg tcccacttgg tggggccagg gtcctctccc 2220
ctgcccagga atgtgcaagg ccagggcatg ggggcaaata tgacccagtt ttgggaacac 2280
cgacaaaccc agccctggcg ctgagcctct ctaccccagg tcagacggac agaaagacag 2340
atcacaggta cagggatgag gacaccggct ctgaccagga gtttggggag cttcaggaca 2400
ttgctgtgct ttggggattc cctccacatg ctgcacgcgc atctcgcccc caggggcact 2460
gcctggaaga ttcaggagcc tgggcggcct tcgcttactc tcacctgctt ctgagttgcc 2520
caggagacca ctggcagatg tcccggcgaa gagaagagac acattgttgg aagaagcagc 2580
ccatgacagc tccccttcct gggactcgcc ctcatcctct tcctgctccc cttcctgggg 2640
tgcagcctaa aaggacctat gtcctcacac cattgaaacc actagttctg tccccccagg 2700
agacctggtt gtgtgtgtgt gagtggttga ccttcctcca tcccctggtc cttcccttcc 2760
cttcccgagg cacagagaga cagggcagga tccacgtgcc cattgtggag gcagagaaaa 2820
gagaaagtgt tttatatacg gtacttattt aatatccctt tttaattaga aattaaaaca 2880
gttaatttaa ttaaagagta gggttttttt tcagtattct tggttaatat ttaatttcaa 2940
ctatttatga gatgtatctt ttgctctctc ttgctctctt atttgtaccg gtttttgtat 3000
ataaaattca tgtttccaat ctctctctcc ctgatcggtg acagtcacta gcttatcttg 3060
aacagatatt taattttgct aacactcagc tctgccctcc ccgatcccct ggctccccag 3120
cacacattcc tttgaaataa ggtttcaata tacatctaca tactatatat atatttggca 3180
acttgtattt gtgtgtatat atatatatat atgtttatgt atatatgtga ttctgataaa 3240
atagacattg ctattctgtt ttttatatgt aaaaacaaaa caagaaaaaa tagagaattc 3300
tacatactaa atctctctcc ttttttaatt ttaatatttg ttatcattta tttattggtg 3360
ctactgttta tccgtaataa ttgtggggaa aagatattaa catcacgtct ttgtctctag 3420
tgcagttttt cgagatattc cgtagtacat atttattttt aaacaacgac aaagaaatac 3480
agatatatct taaaaaaaaa aaagcatttt gtattaaaga atttaattct gatctcaaaa 3540
aaaaaaaaaa aaaa 3554
<210> SEQ ID NO 19
<211> LENGTH: 3519
<212> TYPE: DNA
<213> ORGANISM: Homo sapiens
<400> SEQUENCE: 19
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa tccctgtggg ccttgctcag agcggagaaa gcatttgttt 1500
gtacaagatc cgcagacgtg taaatgttcc tgcaaaaaca cagactcgcg ttgcaagatg 1560
tgacaagccg aggcggtgag ccgggcagga ggaaggagcc tccctcaggg tttcgggaac 1620
cagatctctc accaggaaag actgatacag aacgatcgat acagaaacca cgctgccgcc 1680
accacaccat caccatcgac agaacagtcc ttaatccaga aacctgaaat gaaggaagag 1740
gagactctgc gcagagcact ttgggtccgg agggcgagac tccggcggaa gcattcccgg 1800
gcgggtgacc cagcacggtc cctcttggaa ttggattcgc cattttattt ttcttgctgc 1860
taaatcaccg agcccggaag attagagagt tttatttctg ggattcctgt agacacaccc 1920
acccacatac atacatttat atatatatat attatatata tataaaaata aatatctcta 1980
ttttatatat ataaaatata tatattcttt ttttaaatta acagtgctaa tgttattggt 2040
gtcttcactg gatgtatttg actgctgtgg acttgagttg ggaggggaat gttcccactc 2100
agatcctgac agggaagagg aggagatgag agactctggc atgatctttt ttttgtccca 2160
cttggtgggg ccagggtcct ctcccctgcc caggaatgtg caaggccagg gcatgggggc 2220
aaatatgacc cagttttggg aacaccgaca aacccagccc tggcgctgag cctctctacc 2280
ccaggtcaga cggacagaaa gacagatcac aggtacaggg atgaggacac cggctctgac 2340
caggagtttg gggagcttca ggacattgct gtgctttggg gattccctcc acatgctgca 2400
cgcgcatctc gcccccaggg gcactgcctg gaagattcag gagcctgggc ggccttcgct 2460
tactctcacc tgcttctgag ttgcccagga gaccactggc agatgtcccg gcgaagagaa 2520
gagacacatt gttggaagaa gcagcccatg acagctcccc ttcctgggac tcgccctcat 2580
cctcttcctg ctccccttcc tggggtgcag cctaaaagga cctatgtcct cacaccattg 2640
aaaccactag ttctgtcccc ccaggagacc tggttgtgtg tgtgtgagtg gttgaccttc 2700
ctccatcccc tggtccttcc cttcccttcc cgaggcacag agagacaggg caggatccac 2760
gtgcccattg tggaggcaga gaaaagagaa agtgttttat atacggtact tatttaatat 2820
ccctttttaa ttagaaatta aaacagttaa tttaattaaa gagtagggtt ttttttcagt 2880
attcttggtt aatatttaat ttcaactatt tatgagatgt atcttttgct ctctcttgct 2940
ctcttatttg taccggtttt tgtatataaa attcatgttt ccaatctctc tctccctgat 3000
cggtgacagt cactagctta tcttgaacag atatttaatt ttgctaacac tcagctctgc 3060
cctccccgat cccctggctc cccagcacac attcctttga aataaggttt caatatacat 3120
ctacatacta tatatatatt tggcaacttg tatttgtgtg tatatatata tatatatgtt 3180
tatgtatata tgtgattctg ataaaataga cattgctatt ctgtttttta tatgtaaaaa 3240
caaaacaaga aaaaatagag aattctacat actaaatctc tctccttttt taattttaat 3300
atttgttatc atttatttat tggtgctact gtttatccgt aataattgtg gggaaaagat 3360
attaacatca cgtctttgtc tctagtgcag tttttcgaga tattccgtag tacatattta 3420
tttttaaaca acgacaaaga aatacagata tatcttaaaa aaaaaaaagc attttgtatt 3480
aaagaattta attctgatct caaaaaaaaa aaaaaaaaa 3519
<210> SEQ ID NO 20
<211> LENGTH: 3519
<212> TYPE: DNA
<213> ORGANISM: Homo sapiens
<400> SEQUENCE: 20
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa tccctgtggg ccttgctcag agcggagaaa gcatttgttt 1500
gtacaagatc cgcagacgtg taaatgttcc tgcaaaaaca cagactcgcg ttgcaagatg 1560
tgacaagccg aggcggtgag ccgggcagga ggaaggagcc tccctcaggg tttcgggaac 1620
cagatctctc accaggaaag actgatacag aacgatcgat acagaaacca cgctgccgcc 1680
accacaccat caccatcgac agaacagtcc ttaatccaga aacctgaaat gaaggaagag 1740
gagactctgc gcagagcact ttgggtccgg agggcgagac tccggcggaa gcattcccgg 1800
gcgggtgacc cagcacggtc cctcttggaa ttggattcgc cattttattt ttcttgctgc 1860
taaatcaccg agcccggaag attagagagt tttatttctg ggattcctgt agacacaccc 1920
acccacatac atacatttat atatatatat attatatata tataaaaata aatatctcta 1980
ttttatatat ataaaatata tatattcttt ttttaaatta acagtgctaa tgttattggt 2040
gtcttcactg gatgtatttg actgctgtgg acttgagttg ggaggggaat gttcccactc 2100
agatcctgac agggaagagg aggagatgag agactctggc atgatctttt ttttgtccca 2160
cttggtgggg ccagggtcct ctcccctgcc caggaatgtg caaggccagg gcatgggggc 2220
aaatatgacc cagttttggg aacaccgaca aacccagccc tggcgctgag cctctctacc 2280
ccaggtcaga cggacagaaa gacagatcac aggtacaggg atgaggacac cggctctgac 2340
caggagtttg gggagcttca ggacattgct gtgctttggg gattccctcc acatgctgca 2400
cgcgcatctc gcccccaggg gcactgcctg gaagattcag gagcctgggc ggccttcgct 2460
tactctcacc tgcttctgag ttgcccagga gaccactggc agatgtcccg gcgaagagaa 2520
gagacacatt gttggaagaa gcagcccatg acagctcccc ttcctgggac tcgccctcat 2580
cctcttcctg ctccccttcc tggggtgcag cctaaaagga cctatgtcct cacaccattg 2640
aaaccactag ttctgtcccc ccaggagacc tggttgtgtg tgtgtgagtg gttgaccttc 2700
ctccatcccc tggtccttcc cttcccttcc cgaggcacag agagacaggg caggatccac 2760
gtgcccattg tggaggcaga gaaaagagaa agtgttttat atacggtact tatttaatat 2820
ccctttttaa ttagaaatta aaacagttaa tttaattaaa gagtagggtt ttttttcagt 2880
attcttggtt aatatttaat ttcaactatt tatgagatgt atcttttgct ctctcttgct 2940
ctcttatttg taccggtttt tgtatataaa attcatgttt ccaatctctc tctccctgat 3000
cggtgacagt cactagctta tcttgaacag atatttaatt ttgctaacac tcagctctgc 3060
cctccccgat cccctggctc cccagcacac attcctttga aataaggttt caatatacat 3120
ctacatacta tatatatatt tggcaacttg tatttgtgtg tatatatata tatatatgtt 3180
tatgtatata tgtgattctg ataaaataga cattgctatt ctgtttttta tatgtaaaaa 3240
caaaacaaga aaaaatagag aattctacat actaaatctc tctccttttt taattttaat 3300
atttgttatc atttatttat tggtgctact gtttatccgt aataattgtg gggaaaagat 3360
attaacatca cgtctttgtc tctagtgcag tttttcgaga tattccgtag tacatattta 3420
tttttaaaca acgacaaaga aatacagata tatcttaaaa aaaaaaaagc attttgtatt 3480
aaagaattta attctgatct caaaaaaaaa aaaaaaaaa 3519
<210> SEQ ID NO 21
<211> LENGTH: 3422
<212> TYPE: DNA
<213> ORGANISM: Homo sapiens
<400> SEQUENCE: 21
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa atgtgacaag ccgaggcggt gagccgggca ggaggaagga 1500
gcctccctca gggtttcggg aaccagatct ctcaccagga aagactgata cagaacgatc 1560
gatacagaaa ccacgctgcc gccaccacac catcaccatc gacagaacag tccttaatcc 1620
agaaacctga aatgaaggaa gaggagactc tgcgcagagc actttgggtc cggagggcga 1680
gactccggcg gaagcattcc cgggcgggtg acccagcacg gtccctcttg gaattggatt 1740
cgccatttta tttttcttgc tgctaaatca ccgagcccgg aagattagag agttttattt 1800
ctgggattcc tgtagacaca cccacccaca tacatacatt tatatatata tatattatat 1860
atatataaaa ataaatatct ctattttata tatataaaat atatatattc tttttttaaa 1920
ttaacagtgc taatgttatt ggtgtcttca ctggatgtat ttgactgctg tggacttgag 1980
ttgggagggg aatgttccca ctcagatcct gacagggaag aggaggagat gagagactct 2040
ggcatgatct tttttttgtc ccacttggtg gggccagggt cctctcccct gcccaggaat 2100
gtgcaaggcc agggcatggg ggcaaatatg acccagtttt gggaacaccg acaaacccag 2160
ccctggcgct gagcctctct accccaggtc agacggacag aaagacagat cacaggtaca 2220
gggatgagga caccggctct gaccaggagt ttggggagct tcaggacatt gctgtgcttt 2280
ggggattccc tccacatgct gcacgcgcat ctcgccccca ggggcactgc ctggaagatt 2340
caggagcctg ggcggccttc gcttactctc acctgcttct gagttgccca ggagaccact 2400
ggcagatgtc ccggcgaaga gaagagacac attgttggaa gaagcagccc atgacagctc 2460
cccttcctgg gactcgccct catcctcttc ctgctcccct tcctggggtg cagcctaaaa 2520
ggacctatgt cctcacacca ttgaaaccac tagttctgtc cccccaggag acctggttgt 2580
gtgtgtgtga gtggttgacc ttcctccatc ccctggtcct tcccttccct tcccgaggca 2640
cagagagaca gggcaggatc cacgtgccca ttgtggaggc agagaaaaga gaaagtgttt 2700
tatatacggt acttatttaa tatccctttt taattagaaa ttaaaacagt taatttaatt 2760
aaagagtagg gttttttttc agtattcttg gttaatattt aatttcaact atttatgaga 2820
tgtatctttt gctctctctt gctctcttat ttgtaccggt ttttgtatat aaaattcatg 2880
tttccaatct ctctctccct gatcggtgac agtcactagc ttatcttgaa cagatattta 2940
attttgctaa cactcagctc tgccctcccc gatcccctgg ctccccagca cacattcctt 3000
tgaaataagg tttcaatata catctacata ctatatatat atttggcaac ttgtatttgt 3060
gtgtatatat atatatatat gtttatgtat atatgtgatt ctgataaaat agacattgct 3120
attctgtttt ttatatgtaa aaacaaaaca agaaaaaata gagaattcta catactaaat 3180
ctctctcctt ttttaatttt aatatttgtt atcatttatt tattggtgct actgtttatc 3240
cgtaataatt gtggggaaaa gatattaaca tcacgtcttt gtctctagtg cagtttttcg 3300
agatattccg tagtacatat ttatttttaa acaacgacaa agaaatacag atatatctta 3360
aaaaaaaaaa agcattttgt attaaagaat ttaattctga tctcaaaaaa aaaaaaaaaa 3420
aa 3422
<210> SEQ ID NO 22
<211> LENGTH: 3422
<212> TYPE: DNA
<213> ORGANISM: Homo sapiens
<400> SEQUENCE: 22
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa atgtgacaag ccgaggcggt gagccgggca ggaggaagga 1500
gcctccctca gggtttcggg aaccagatct ctcaccagga aagactgata cagaacgatc 1560
gatacagaaa ccacgctgcc gccaccacac catcaccatc gacagaacag tccttaatcc 1620
agaaacctga aatgaaggaa gaggagactc tgcgcagagc actttgggtc cggagggcga 1680
gactccggcg gaagcattcc cgggcgggtg acccagcacg gtccctcttg gaattggatt 1740
cgccatttta tttttcttgc tgctaaatca ccgagcccgg aagattagag agttttattt 1800
ctgggattcc tgtagacaca cccacccaca tacatacatt tatatatata tatattatat 1860
atatataaaa ataaatatct ctattttata tatataaaat atatatattc tttttttaaa 1920
ttaacagtgc taatgttatt ggtgtcttca ctggatgtat ttgactgctg tggacttgag 1980
ttgggagggg aatgttccca ctcagatcct gacagggaag aggaggagat gagagactct 2040
ggcatgatct tttttttgtc ccacttggtg gggccagggt cctctcccct gcccaggaat 2100
gtgcaaggcc agggcatggg ggcaaatatg acccagtttt gggaacaccg acaaacccag 2160
ccctggcgct gagcctctct accccaggtc agacggacag aaagacagat cacaggtaca 2220
gggatgagga caccggctct gaccaggagt ttggggagct tcaggacatt gctgtgcttt 2280
ggggattccc tccacatgct gcacgcgcat ctcgccccca ggggcactgc ctggaagatt 2340
caggagcctg ggcggccttc gcttactctc acctgcttct gagttgccca ggagaccact 2400
ggcagatgtc ccggcgaaga gaagagacac attgttggaa gaagcagccc atgacagctc 2460
cccttcctgg gactcgccct catcctcttc ctgctcccct tcctggggtg cagcctaaaa 2520
ggacctatgt cctcacacca ttgaaaccac tagttctgtc cccccaggag acctggttgt 2580
gtgtgtgtga gtggttgacc ttcctccatc ccctggtcct tcccttccct tcccgaggca 2640
cagagagaca gggcaggatc cacgtgccca ttgtggaggc agagaaaaga gaaagtgttt 2700
tatatacggt acttatttaa tatccctttt taattagaaa ttaaaacagt taatttaatt 2760
aaagagtagg gttttttttc agtattcttg gttaatattt aatttcaact atttatgaga 2820
tgtatctttt gctctctctt gctctcttat ttgtaccggt ttttgtatat aaaattcatg 2880
tttccaatct ctctctccct gatcggtgac agtcactagc ttatcttgaa cagatattta 2940
attttgctaa cactcagctc tgccctcccc gatcccctgg ctccccagca cacattcctt 3000
tgaaataagg tttcaatata catctacata ctatatatat atttggcaac ttgtatttgt 3060
gtgtatatat atatatatat gtttatgtat atatgtgatt ctgataaaat agacattgct 3120
attctgtttt ttatatgtaa aaacaaaaca agaaaaaata gagaattcta catactaaat 3180
ctctctcctt ttttaatttt aatatttgtt atcatttatt tattggtgct actgtttatc 3240
cgtaataatt gtggggaaaa gatattaaca tcacgtcttt gtctctagtg cagtttttcg 3300
agatattccg tagtacatat ttatttttaa acaacgacaa agaaatacag atatatctta 3360
aaaaaaaaaa agcattttgt attaaagaat ttaattctga tctcaaaaaa aaaaaaaaaa 3420
aa 3422
<210> SEQ ID NO 23
<211> LENGTH: 3488
<212> TYPE: DNA
<213> ORGANISM: Homo sapiens
<400> SEQUENCE: 23
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa tccctgtggg ccttgctcag agcggagaaa gcatttgttt 1500
gtacaagatc cgcagacgtg taaatgttcc tgcaaaaaca cagactcgcg ttgcaaggcg 1560
aggcagcttg agttaaacga acgtacttgc agatctctca ccaggaaaga ctgatacaga 1620
acgatcgata cagaaaccac gctgccgcca ccacaccatc accatcgaca gaacagtcct 1680
taatccagaa acctgaaatg aaggaagagg agactctgcg cagagcactt tgggtccgga 1740
gggcgagact ccggcggaag cattcccggg cgggtgaccc agcacggtcc ctcttggaat 1800
tggattcgcc attttatttt tcttgctgct aaatcaccga gcccggaaga ttagagagtt 1860
ttatttctgg gattcctgta gacacaccca cccacataca tacatttata tatatatata 1920
ttatatatat ataaaaataa atatctctat tttatatata taaaatatat atattctttt 1980
tttaaattaa cagtgctaat gttattggtg tcttcactgg atgtatttga ctgctgtgga 2040
cttgagttgg gaggggaatg ttcccactca gatcctgaca gggaagagga ggagatgaga 2100
gactctggca tgatcttttt tttgtcccac ttggtggggc cagggtcctc tcccctgccc 2160
aggaatgtgc aaggccaggg catgggggca aatatgaccc agttttggga acaccgacaa 2220
acccagccct ggcgctgagc ctctctaccc caggtcagac ggacagaaag acagatcaca 2280
ggtacaggga tgaggacacc ggctctgacc aggagtttgg ggagcttcag gacattgctg 2340
tgctttgggg attccctcca catgctgcac gcgcatctcg cccccagggg cactgcctgg 2400
aagattcagg agcctgggcg gccttcgctt actctcacct gcttctgagt tgcccaggag 2460
accactggca gatgtcccgg cgaagagaag agacacattg ttggaagaag cagcccatga 2520
cagctcccct tcctgggact cgccctcatc ctcttcctgc tccccttcct ggggtgcagc 2580
ctaaaaggac ctatgtcctc acaccattga aaccactagt tctgtccccc caggagacct 2640
ggttgtgtgt gtgtgagtgg ttgaccttcc tccatcccct ggtccttccc ttcccttccc 2700
gaggcacaga gagacagggc aggatccacg tgcccattgt ggaggcagag aaaagagaaa 2760
gtgttttata tacggtactt atttaatatc cctttttaat tagaaattaa aacagttaat 2820
ttaattaaag agtagggttt tttttcagta ttcttggtta atatttaatt tcaactattt 2880
atgagatgta tcttttgctc tctcttgctc tcttatttgt accggttttt gtatataaaa 2940
ttcatgtttc caatctctct ctccctgatc ggtgacagtc actagcttat cttgaacaga 3000
tatttaattt tgctaacact cagctctgcc ctccccgatc ccctggctcc ccagcacaca 3060
ttcctttgaa ataaggtttc aatatacatc tacatactat atatatattt ggcaacttgt 3120
atttgtgtgt atatatatat atatatgttt atgtatatat gtgattctga taaaatagac 3180
attgctattc tgttttttat atgtaaaaac aaaacaagaa aaaatagaga attctacata 3240
ctaaatctct ctcctttttt aattttaata tttgttatca tttatttatt ggtgctactg 3300
tttatccgta ataattgtgg ggaaaagata ttaacatcac gtctttgtct ctagtgcagt 3360
ttttcgagat attccgtagt acatatttat ttttaaacaa cgacaaagaa atacagatat 3420
atcttaaaaa aaaaaaagca ttttgtatta aagaatttaa ttctgatctc aaaaaaaaaa 3480
aaaaaaaa 3488
<210> SEQ ID NO 24
<211> LENGTH: 3488
<212> TYPE: DNA
<213> ORGANISM: Homo sapiens
<400> SEQUENCE: 24
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa tccctgtggg ccttgctcag agcggagaaa gcatttgttt 1500
gtacaagatc cgcagacgtg taaatgttcc tgcaaaaaca cagactcgcg ttgcaaggcg 1560
aggcagcttg agttaaacga acgtacttgc agatctctca ccaggaaaga ctgatacaga 1620
acgatcgata cagaaaccac gctgccgcca ccacaccatc accatcgaca gaacagtcct 1680
taatccagaa acctgaaatg aaggaagagg agactctgcg cagagcactt tgggtccgga 1740
gggcgagact ccggcggaag cattcccggg cgggtgaccc agcacggtcc ctcttggaat 1800
tggattcgcc attttatttt tcttgctgct aaatcaccga gcccggaaga ttagagagtt 1860
ttatttctgg gattcctgta gacacaccca cccacataca tacatttata tatatatata 1920
ttatatatat ataaaaataa atatctctat tttatatata taaaatatat atattctttt 1980
tttaaattaa cagtgctaat gttattggtg tcttcactgg atgtatttga ctgctgtgga 2040
cttgagttgg gaggggaatg ttcccactca gatcctgaca gggaagagga ggagatgaga 2100
gactctggca tgatcttttt tttgtcccac ttggtggggc cagggtcctc tcccctgccc 2160
aggaatgtgc aaggccaggg catgggggca aatatgaccc agttttggga acaccgacaa 2220
acccagccct ggcgctgagc ctctctaccc caggtcagac ggacagaaag acagatcaca 2280
ggtacaggga tgaggacacc ggctctgacc aggagtttgg ggagcttcag gacattgctg 2340
tgctttgggg attccctcca catgctgcac gcgcatctcg cccccagggg cactgcctgg 2400
aagattcagg agcctgggcg gccttcgctt actctcacct gcttctgagt tgcccaggag 2460
accactggca gatgtcccgg cgaagagaag agacacattg ttggaagaag cagcccatga 2520
cagctcccct tcctgggact cgccctcatc ctcttcctgc tccccttcct ggggtgcagc 2580
ctaaaaggac ctatgtcctc acaccattga aaccactagt tctgtccccc caggagacct 2640
ggttgtgtgt gtgtgagtgg ttgaccttcc tccatcccct ggtccttccc ttcccttccc 2700
gaggcacaga gagacagggc aggatccacg tgcccattgt ggaggcagag aaaagagaaa 2760
gtgttttata tacggtactt atttaatatc cctttttaat tagaaattaa aacagttaat 2820
ttaattaaag agtagggttt tttttcagta ttcttggtta atatttaatt tcaactattt 2880
atgagatgta tcttttgctc tctcttgctc tcttatttgt accggttttt gtatataaaa 2940
ttcatgtttc caatctctct ctccctgatc ggtgacagtc actagcttat cttgaacaga 3000
tatttaattt tgctaacact cagctctgcc ctccccgatc ccctggctcc ccagcacaca 3060
ttcctttgaa ataaggtttc aatatacatc tacatactat atatatattt ggcaacttgt 3120
atttgtgtgt atatatatat atatatgttt atgtatatat gtgattctga taaaatagac 3180
attgctattc tgttttttat atgtaaaaac aaaacaagaa aaaatagaga attctacata 3240
ctaaatctct ctcctttttt aattttaata tttgttatca tttatttatt ggtgctactg 3300
tttatccgta ataattgtgg ggaaaagata ttaacatcac gtctttgtct ctagtgcagt 3360
ttttcgagat attccgtagt acatatttat ttttaaacaa cgacaaagaa atacagatat 3420
atcttaaaaa aaaaaaagca ttttgtatta aagaatttaa ttctgatctc aaaaaaaaaa 3480
aaaaaaaa 3488
<210> SEQ ID NO 25
<211> LENGTH: 3392
<212> TYPE: DNA
<213> ORGANISM: Homo sapiens
<400> SEQUENCE: 25
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag atgtgacaag 1440
ccgaggcggt gagccgggca ggaggaagga gcctccctca gggtttcggg aaccagatct 1500
ctcaccagga aagactgata cagaacgatc gatacagaaa ccacgctgcc gccaccacac 1560
catcaccatc gacagaacag tccttaatcc agaaacctga aatgaaggaa gaggagactc 1620
tgcgcagagc actttgggtc cggagggcga gactccggcg gaagcattcc cgggcgggtg 1680
acccagcacg gtccctcttg gaattggatt cgccatttta tttttcttgc tgctaaatca 1740
ccgagcccgg aagattagag agttttattt ctgggattcc tgtagacaca cccacccaca 1800
tacatacatt tatatatata tatattatat atatataaaa ataaatatct ctattttata 1860
tatataaaat atatatattc tttttttaaa ttaacagtgc taatgttatt ggtgtcttca 1920
ctggatgtat ttgactgctg tggacttgag ttgggagggg aatgttccca ctcagatcct 1980
gacagggaag aggaggagat gagagactct ggcatgatct tttttttgtc ccacttggtg 2040
gggccagggt cctctcccct gcccaggaat gtgcaaggcc agggcatggg ggcaaatatg 2100
acccagtttt gggaacaccg acaaacccag ccctggcgct gagcctctct accccaggtc 2160
agacggacag aaagacagat cacaggtaca gggatgagga caccggctct gaccaggagt 2220
ttggggagct tcaggacatt gctgtgcttt ggggattccc tccacatgct gcacgcgcat 2280
ctcgccccca ggggcactgc ctggaagatt caggagcctg ggcggccttc gcttactctc 2340
acctgcttct gagttgccca ggagaccact ggcagatgtc ccggcgaaga gaagagacac 2400
attgttggaa gaagcagccc atgacagctc cccttcctgg gactcgccct catcctcttc 2460
ctgctcccct tcctggggtg cagcctaaaa ggacctatgt cctcacacca ttgaaaccac 2520
tagttctgtc cccccaggag acctggttgt gtgtgtgtga gtggttgacc ttcctccatc 2580
ccctggtcct tcccttccct tcccgaggca cagagagaca gggcaggatc cacgtgccca 2640
ttgtggaggc agagaaaaga gaaagtgttt tatatacggt acttatttaa tatccctttt 2700
taattagaaa ttaaaacagt taatttaatt aaagagtagg gttttttttc agtattcttg 2760
gttaatattt aatttcaact atttatgaga tgtatctttt gctctctctt gctctcttat 2820
ttgtaccggt ttttgtatat aaaattcatg tttccaatct ctctctccct gatcggtgac 2880
agtcactagc ttatcttgaa cagatattta attttgctaa cactcagctc tgccctcccc 2940
gatcccctgg ctccccagca cacattcctt tgaaataagg tttcaatata catctacata 3000
ctatatatat atttggcaac ttgtatttgt gtgtatatat atatatatat gtttatgtat 3060
atatgtgatt ctgataaaat agacattgct attctgtttt ttatatgtaa aaacaaaaca 3120
agaaaaaata gagaattcta catactaaat ctctctcctt ttttaatttt aatatttgtt 3180
atcatttatt tattggtgct actgtttatc cgtaataatt gtggggaaaa gatattaaca 3240
tcacgtcttt gtctctagtg cagtttttcg agatattccg tagtacatat ttatttttaa 3300
acaacgacaa agaaatacag atatatctta aaaaaaaaaa agcattttgt attaaagaat 3360
ttaattctga tctcaaaaaa aaaaaaaaaa aa 3392
<210> SEQ ID NO 26
<211> LENGTH: 3392
<212> TYPE: DNA
<213> ORGANISM: Homo sapiens
<400> SEQUENCE: 26
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag atgtgacaag 1440
ccgaggcggt gagccgggca ggaggaagga gcctccctca gggtttcggg aaccagatct 1500
ctcaccagga aagactgata cagaacgatc gatacagaaa ccacgctgcc gccaccacac 1560
catcaccatc gacagaacag tccttaatcc agaaacctga aatgaaggaa gaggagactc 1620
tgcgcagagc actttgggtc cggagggcga gactccggcg gaagcattcc cgggcgggtg 1680
acccagcacg gtccctcttg gaattggatt cgccatttta tttttcttgc tgctaaatca 1740
ccgagcccgg aagattagag agttttattt ctgggattcc tgtagacaca cccacccaca 1800
tacatacatt tatatatata tatattatat atatataaaa ataaatatct ctattttata 1860
tatataaaat atatatattc tttttttaaa ttaacagtgc taatgttatt ggtgtcttca 1920
ctggatgtat ttgactgctg tggacttgag ttgggagggg aatgttccca ctcagatcct 1980
gacagggaag aggaggagat gagagactct ggcatgatct tttttttgtc ccacttggtg 2040
gggccagggt cctctcccct gcccaggaat gtgcaaggcc agggcatggg ggcaaatatg 2100
acccagtttt gggaacaccg acaaacccag ccctggcgct gagcctctct accccaggtc 2160
agacggacag aaagacagat cacaggtaca gggatgagga caccggctct gaccaggagt 2220
ttggggagct tcaggacatt gctgtgcttt ggggattccc tccacatgct gcacgcgcat 2280
ctcgccccca ggggcactgc ctggaagatt caggagcctg ggcggccttc gcttactctc 2340
acctgcttct gagttgccca ggagaccact ggcagatgtc ccggcgaaga gaagagacac 2400
attgttggaa gaagcagccc atgacagctc cccttcctgg gactcgccct catcctcttc 2460
ctgctcccct tcctggggtg cagcctaaaa ggacctatgt cctcacacca ttgaaaccac 2520
tagttctgtc cccccaggag acctggttgt gtgtgtgtga gtggttgacc ttcctccatc 2580
ccctggtcct tcccttccct tcccgaggca cagagagaca gggcaggatc cacgtgccca 2640
ttgtggaggc agagaaaaga gaaagtgttt tatatacggt acttatttaa tatccctttt 2700
taattagaaa ttaaaacagt taatttaatt aaagagtagg gttttttttc agtattcttg 2760
gttaatattt aatttcaact atttatgaga tgtatctttt gctctctctt gctctcttat 2820
ttgtaccggt ttttgtatat aaaattcatg tttccaatct ctctctccct gatcggtgac 2880
agtcactagc ttatcttgaa cagatattta attttgctaa cactcagctc tgccctcccc 2940
gatcccctgg ctccccagca cacattcctt tgaaataagg tttcaatata catctacata 3000
ctatatatat atttggcaac ttgtatttgt gtgtatatat atatatatat gtttatgtat 3060
atatgtgatt ctgataaaat agacattgct attctgtttt ttatatgtaa aaacaaaaca 3120
agaaaaaata gagaattcta catactaaat ctctctcctt ttttaatttt aatatttgtt 3180
atcatttatt tattggtgct actgtttatc cgtaataatt gtggggaaaa gatattaaca 3240
tcacgtcttt gtctctagtg cagtttttcg agatattccg tagtacatat ttatttttaa 3300
acaacgacaa agaaatacag atatatctta aaaaaaaaaa agcattttgt attaaagaat 3360
ttaattctga tctcaaaaaa aaaaaaaaaa aa 3392
<210> SEQ ID NO 27
<211> LENGTH: 3494
<212> TYPE: DNA
<213> ORGANISM: Homo sapiens
<400> SEQUENCE: 27
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa aaaatcagtt cgaggaaagg gaaaggggca aaaacgaaag 1500
cgcaagaaat cccggtataa gtcctggagc gtatgtgaca agccgaggcg gtgagccggg 1560
caggaggaag gagcctccct cagggtttcg ggaaccagat ctctcaccag gaaagactga 1620
tacagaacga tcgatacaga aaccacgctg ccgccaccac accatcacca tcgacagaac 1680
agtccttaat ccagaaacct gaaatgaagg aagaggagac tctgcgcaga gcactttggg 1740
tccggagggc gagactccgg cggaagcatt cccgggcggg tgacccagca cggtccctct 1800
tggaattgga ttcgccattt tatttttctt gctgctaaat caccgagccc ggaagattag 1860
agagttttat ttctgggatt cctgtagaca cacccaccca catacataca tttatatata 1920
tatatattat atatatataa aaataaatat ctctatttta tatatataaa atatatatat 1980
tcttttttta aattaacagt gctaatgtta ttggtgtctt cactggatgt atttgactgc 2040
tgtggacttg agttgggagg ggaatgttcc cactcagatc ctgacaggga agaggaggag 2100
atgagagact ctggcatgat cttttttttg tcccacttgg tggggccagg gtcctctccc 2160
ctgcccagga atgtgcaagg ccagggcatg ggggcaaata tgacccagtt ttgggaacac 2220
cgacaaaccc agccctggcg ctgagcctct ctaccccagg tcagacggac agaaagacag 2280
atcacaggta cagggatgag gacaccggct ctgaccagga gtttggggag cttcaggaca 2340
ttgctgtgct ttggggattc cctccacatg ctgcacgcgc atctcgcccc caggggcact 2400
gcctggaaga ttcaggagcc tgggcggcct tcgcttactc tcacctgctt ctgagttgcc 2460
caggagacca ctggcagatg tcccggcgaa gagaagagac acattgttgg aagaagcagc 2520
ccatgacagc tccccttcct gggactcgcc ctcatcctct tcctgctccc cttcctgggg 2580
tgcagcctaa aaggacctat gtcctcacac cattgaaacc actagttctg tccccccagg 2640
agacctggtt gtgtgtgtgt gagtggttga ccttcctcca tcccctggtc cttcccttcc 2700
cttcccgagg cacagagaga cagggcagga tccacgtgcc cattgtggag gcagagaaaa 2760
gagaaagtgt tttatatacg gtacttattt aatatccctt tttaattaga aattaaaaca 2820
gttaatttaa ttaaagagta gggttttttt tcagtattct tggttaatat ttaatttcaa 2880
ctatttatga gatgtatctt ttgctctctc ttgctctctt atttgtaccg gtttttgtat 2940
ataaaattca tgtttccaat ctctctctcc ctgatcggtg acagtcacta gcttatcttg 3000
aacagatatt taattttgct aacactcagc tctgccctcc ccgatcccct ggctccccag 3060
cacacattcc tttgaaataa ggtttcaata tacatctaca tactatatat atatttggca 3120
acttgtattt gtgtgtatat atatatatat atgtttatgt atatatgtga ttctgataaa 3180
atagacattg ctattctgtt ttttatatgt aaaaacaaaa caagaaaaaa tagagaattc 3240
tacatactaa atctctctcc ttttttaatt ttaatatttg ttatcattta tttattggtg 3300
ctactgttta tccgtaataa ttgtggggaa aagatattaa catcacgtct ttgtctctag 3360
tgcagttttt cgagatattc cgtagtacat atttattttt aaacaacgac aaagaaatac 3420
agatatatct taaaaaaaaa aaagcatttt gtattaaaga atttaattct gatctcaaaa 3480
aaaaaaaaaa aaaa 3494
<210> SEQ ID NO 28
<211> LENGTH: 3494
<212> TYPE: DNA
<213> ORGANISM: Homo sapiens
<400> SEQUENCE: 28
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa aaaatcagtt cgaggaaagg gaaaggggca aaaacgaaag 1500
cgcaagaaat cccggtataa gtcctggagc gtatgtgaca agccgaggcg gtgagccggg 1560
caggaggaag gagcctccct cagggtttcg ggaaccagat ctctcaccag gaaagactga 1620
tacagaacga tcgatacaga aaccacgctg ccgccaccac accatcacca tcgacagaac 1680
agtccttaat ccagaaacct gaaatgaagg aagaggagac tctgcgcaga gcactttggg 1740
tccggagggc gagactccgg cggaagcatt cccgggcggg tgacccagca cggtccctct 1800
tggaattgga ttcgccattt tatttttctt gctgctaaat caccgagccc ggaagattag 1860
agagttttat ttctgggatt cctgtagaca cacccaccca catacataca tttatatata 1920
tatatattat atatatataa aaataaatat ctctatttta tatatataaa atatatatat 1980
tcttttttta aattaacagt gctaatgtta ttggtgtctt cactggatgt atttgactgc 2040
tgtggacttg agttgggagg ggaatgttcc cactcagatc ctgacaggga agaggaggag 2100
atgagagact ctggcatgat cttttttttg tcccacttgg tggggccagg gtcctctccc 2160
ctgcccagga atgtgcaagg ccagggcatg ggggcaaata tgacccagtt ttgggaacac 2220
cgacaaaccc agccctggcg ctgagcctct ctaccccagg tcagacggac agaaagacag 2280
atcacaggta cagggatgag gacaccggct ctgaccagga gtttggggag cttcaggaca 2340
ttgctgtgct ttggggattc cctccacatg ctgcacgcgc atctcgcccc caggggcact 2400
gcctggaaga ttcaggagcc tgggcggcct tcgcttactc tcacctgctt ctgagttgcc 2460
caggagacca ctggcagatg tcccggcgaa gagaagagac acattgttgg aagaagcagc 2520
ccatgacagc tccccttcct gggactcgcc ctcatcctct tcctgctccc cttcctgggg 2580
tgcagcctaa aaggacctat gtcctcacac cattgaaacc actagttctg tccccccagg 2640
agacctggtt gtgtgtgtgt gagtggttga ccttcctcca tcccctggtc cttcccttcc 2700
cttcccgagg cacagagaga cagggcagga tccacgtgcc cattgtggag gcagagaaaa 2760
gagaaagtgt tttatatacg gtacttattt aatatccctt tttaattaga aattaaaaca 2820
gttaatttaa ttaaagagta gggttttttt tcagtattct tggttaatat ttaatttcaa 2880
ctatttatga gatgtatctt ttgctctctc ttgctctctt atttgtaccg gtttttgtat 2940
ataaaattca tgtttccaat ctctctctcc ctgatcggtg acagtcacta gcttatcttg 3000
aacagatatt taattttgct aacactcagc tctgccctcc ccgatcccct ggctccccag 3060
cacacattcc tttgaaataa ggtttcaata tacatctaca tactatatat atatttggca 3120
acttgtattt gtgtgtatat atatatatat atgtttatgt atatatgtga ttctgataaa 3180
atagacattg ctattctgtt ttttatatgt aaaaacaaaa caagaaaaaa tagagaattc 3240
tacatactaa atctctctcc ttttttaatt ttaatatttg ttatcattta tttattggtg 3300
ctactgttta tccgtaataa ttgtggggaa aagatattaa catcacgtct ttgtctctag 3360
tgcagttttt cgagatattc cgtagtacat atttattttt aaacaacgac aaagaaatac 3420
agatatatct taaaaaaaaa aaagcatttt gtattaaaga atttaattct gatctcaaaa 3480
aaaaaaaaaa aaaa 3494
<210> SEQ ID NO 29
<211> LENGTH: 3494
<212> TYPE: DNA
<213> ORGANISM: Homo sapiens
<400> SEQUENCE: 29
tcgcggaggc ttggggcagc cgggtagctc ggaggtcgtg gcgctggggg ctagcaccag 60
cgctctgtcg ggaggcgcag cggttaggtg gaccggtcag cggactcacc ggccagggcg 120
ctcggtgctg gaatttgata ttcattgatc cgggttttat ccctcttctt ttttcttaaa 180
catttttttt taaaactgta ttgtttctcg ttttaattta tttttgcttg ccattcccca 240
cttgaatcgg gccgacggct tggggagatt gctctacttc cccaaatcac tgtggatttt 300
ggaaaccagc agaaagagga aagaggtagc aagagctcca gagagaagtc gaggaagaga 360
gagacggggt cagagagagc gcgcgggcgt gcgagcagcg aaagcgacag gggcaaagtg 420
agtgacctgc ttttgggggt gaccgccgga gcgcggcgtg agccctcccc cttgggatcc 480
cgcagctgac cagtcgcgct gacggacaga cagacagaca ccgcccccag ccccagctac 540
cacctcctcc ccggccggcg gcggacagtg gacgcggcgg cgagccgcgg gcaggggccg 600
gagcccgcgc ccggaggcgg ggtggagggg gtcggggctc gcggcgtcgc actgaaactt 660
ttcgtccaac ttctgggctg ttctcgcttc ggaggagccg tggtccgcgc gggggaagcc 720
gagccgagcg gagccgcgag aagtgctagc tcgggccggg aggagccgca gccggaggag 780
ggggaggagg aagaagagaa ggaagaggag agggggccgc agtggcgact cggcgctcgg 840
aagccgggct catggacggg tgaggcggcg gtgtgcgcag acagtgctcc agccgcgcgc 900
gctccccagg ccctggcccg ggcctcgggc cggggaggaa gagtagctcg ccgaggcgcc 960
gaggagagcg ggccgcccca cagcccgagc cggagaggga gcgcgagccg cgccggcccc 1020
ggtcgggcct ccgaaaccat gaactttctg ctgtcttggg tgcattggag ccttgccttg 1080
ctgctctacc tccaccatgc caagtggtcc caggctgcac ccatggcaga aggaggaggg 1140
cagaatcatc acgaagtggt gaagttcatg gatgtctatc agcgcagcta ctgccatcca 1200
atcgagaccc tggtggacat cttccaggag taccctgatg agatcgagta catcttcaag 1260
ccatcctgtg tgcccctgat gcgatgcggg ggctgctgca atgacgaggg cctggagtgt 1320
gtgcccactg aggagtccaa catcaccatg cagattatgc ggatcaaacc tcaccaaggc 1380
cagcacatag gagagatgag cttcctacag cacaacaaat gtgaatgcag accaaagaaa 1440
gatagagcaa gacaagaaaa aaaatcagtt cgaggaaagg gaaaggggca aaaacgaaag 1500
cgcaagaaat cccggtataa gtcctggagc gtatgtgaca agccgaggcg gtgagccggg 1560
caggaggaag gagcctccct cagggtttcg ggaaccagat ctctcaccag gaaagactga 1620
tacagaacga tcgatacaga aaccacgctg ccgccaccac accatcacca tcgacagaac 1680
agtccttaat ccagaaacct gaaatgaagg aagaggagac tctgcgcaga gcactttggg 1740
tccggagggc gagactccgg cggaagcatt cccgggcggg tgacccagca cggtccctct 1800
tggaattgga ttcgccattt tatttttctt gctgctaaat caccgagccc ggaagattag 1860
agagttttat ttctgggatt cctgtagaca cacccaccca catacataca tttatatata 1920
tatatattat atatatataa aaataaatat ctctatttta tatatataaa atatatatat 1980
tcttttttta aattaacagt gctaatgtta ttggtgtctt cactggatgt atttgactgc 2040
tgtggacttg agttgggagg ggaatgttcc cactcagatc ctgacaggga agaggaggag 2100
atgagagact ctggcatgat cttttttttg tcccacttgg tggggccagg gtcctctccc 2160
ctgcccagga atgtgcaagg ccagggcatg ggggcaaata tgacccagtt ttgggaacac 2220
cgacaaaccc agccctggcg ctgagcctct ctaccccagg tcagacggac agaaagacag 2280
atcacaggta cagggatgag gacaccggct ctgaccagga gtttggggag cttcaggaca 2340
ttgctgtgct ttggggattc cctccacatg ctgcacgcgc atctcgcccc caggggcact 2400
gcctggaaga ttcaggagcc tgggcggcct tcgcttactc tcacctgctt ctgagttgcc 2460
caggagacca ctggcagatg tcccggcgaa gagaagagac acattgttgg aagaagcagc 2520
ccatgacagc tccccttcct gggactcgcc ctcatcctct tcctgctccc cttcctgggg 2580
tgcagcctaa aaggacctat gtcctcacac cattgaaacc actagttctg tccccccagg 2640
agacctggtt gtgtgtgtgt gagtggttga ccttcctcca tcccctggtc cttcccttcc 2700
cttcccgagg cacagagaga cagggcagga tccacgtgcc cattgtggag gcagagaaaa 2760
gagaaagtgt tttatatacg gtacttattt aatatccctt tttaattaga aattaaaaca 2820
gttaatttaa ttaaagagta gggttttttt tcagtattct tggttaatat ttaatttcaa 2880
ctatttatga gatgtatctt ttgctctctc ttgctctctt atttgtaccg gtttttgtat 2940
ataaaattca tgtttccaat ctctctctcc ctgatcggtg acagtcacta gcttatcttg 3000
aacagatatt taattttgct aacactcagc tctgccctcc ccgatcccct ggctccccag 3060
cacacattcc tttgaaataa ggtttcaata tacatctaca tactatatat atatttggca 3120
acttgtattt gtgtgtatat atatatatat atgtttatgt atatatgtga ttctgataaa 3180
atagacattg ctattctgtt ttttatatgt aaaaacaaaa caagaaaaaa tagagaattc 3240
tacatactaa atctctctcc ttttttaatt ttaatatttg ttatcattta tttattggtg 3300
ctactgttta tccgtaataa ttgtggggaa aagatattaa catcacgtct ttgtctctag 3360
tgcagttttt cgagatattc cgtagtacat atttattttt aaacaacgac aaagaaatac 3420
agatatatct taaaaaaaaa aaagcatttt gtattaaaga atttaattct gatctcaaaa 3480
aaaaaaaaaa aaaa 3494
<210> SEQ ID NO 30
<211> LENGTH: 1721
<212> TYPE: DNA
<213> ORGANISM: Homo sapiens
<400> SEQUENCE: 30
gccgtccccg ccgccgctgc ccgccgccac cggccgcccg cccgcccggc tcctccggcc 60
gcctccgctg cgctgcgctg cgctgcctgc acccagggct cgggaggggg ccgcggagga 120
gtcgcccccc gcgcccggcc cccgcccgcc gcgcccgggc ccgcgccatg gggctctggc 180
tgtcgccgcc ccccgcgccg ccgggctagg gcgatgcggg cgcccccggc gggcggcccc 240
ggcgggcacc atgagccctc tgctccgccg cctgctgctc gccgcactcc tgcagctggc 300
ccccgcccag gcccctgtct cccagcctga tgcccctggc caccagagga aagtggtgtc 360
atggatagat gtgtatactc gcgctacctg ccagccccgg gaggtggtgg tgcccttgac 420
tgtggagctc atgggcaccg tggccaaaca gctggtgccc agctgcgtga ctgtgcagcg 480
ctgtggtggc tgctgccctg acgatggcct ggagtgtgtg cccactgggc agcaccaagt 540
ccggatgcag atcctcatga tccggtaccc gagcagtcag ctgggggaga tgtccctgga 600
agaacacagc cagtgtgaat gcagacctaa aaaaaaggac agtgctgtga agccagacag 660
ccccaggccc ctctgcccac gctgcaccca gcaccaccag cgccctgacc cccggacctg 720
ccgctgccgc tgccgacgcc gcagcttcct ccgttgccaa gggcggggct tagagctcaa 780
cccagacacc tgcaggtgcc ggaagctgcg aaggtgacac atggcttttc agactcagca 840
gggtgacttg cctcagaggc tatatcccag tgggggaaca aagaggagcc tggtaaaaaa 900
cagccaagcc cccaagacct cagcccaggc agaagctgct ctaggacctg ggcctctcag 960
agggctcttc tgccatccct tgtctccctg aggccatcat caaacaggac agagttggaa 1020
gaggagactg ggaggcagca agaggggtca cataccagct caggggagaa tggagtactg 1080
tctcagtttc taaccactct gtgcaagtaa gcatcttaca actggctctt cctcccctca 1140
ctaagaagac ccaaacctct gcataatggg atttgggctt tggtacaaga actgtgaccc 1200
ccaaccctga taaaagagat ggaaggagct gtccctgcct gtgtcactgt ttgtcactgt 1260
ccaggctggc tggtttgggc atgaatgtct gcatcactaa atccagagct tgtcttgctc 1320
cctcattgtg cagatggagg aaatgaggac taaggcccca cagcagatcc caggcagggc 1380
cagaattatg tattcatcac tttcaagtta ttgccacgca tgggagtcag ggatagccca 1440
gtcaatacag actgcctgcc ctcctgctct tcaccagggt tcttttctag aaggagacag 1500
ccttctgtgg ccagagagct tggggtagga cccagatcta ctgagtgacc ttgcttgtca 1560
ctacccctgc ctctctgagc agcagtttcc acatgtgcac atagagggaa cagaagattg 1620
ctgtggttgg cgtcctcggg ccccagagaa gtttgagact atctttacgt aatagaaaag 1680
aacacttgtt cttcctgcca ggcaaaaaaa aaaaaaaaaa a 1721
<210> SEQ ID NO 31
<211> LENGTH: 2076
<212> TYPE: DNA
<213> ORGANISM: Homo sapiens
<400> SEQUENCE: 31
cggggaaggg gagggaggag ggggacgagg gctctggcgg gtttggaggg gctgaacatc 60
gcggggtgtt ctggtgtccc ccgccccgcc tctccaaaaa gctacaccga cgcggaccgc 120
ggcggcgtcc tccctcgccc tcgcttcacc tcgcgggctc cgaatgcggg gagctcggat 180
gtccggtttc ctgtgaggct tttacctgac acccgccgcc tttccccggc actggctggg 240
agggcgccct gcaaagttgg gaacgcggag ccccggaccc gctcccgccg cctccggctc 300
gcccaggggg ggtcgccggg aggagcccgg gggagaggga ccaggagggg cccgcggcct 360
cgcaggggcg cccgcgcccc cacccctgcc cccgccagcg gaccggtccc ccacccccgg 420
tccttccacc atgcacttgc tgggcttctt ctctgtggcg tgttctctgc tcgccgctgc 480
gctgctcccg ggtcctcgcg aggcgcccgc cgccgccgcc gccttcgagt ccggactcga 540
cctctcggac gcggagcccg acgcgggcga ggccacggct tatgcaagca aagatctgga 600
ggagcagtta cggtctgtgt ccagtgtaga tgaactcatg actgtactct acccagaata 660
ttggaaaatg tacaagtgtc agctaaggaa aggaggctgg caacataaca gagaacaggc 720
caacctcaac tcaaggacag aagagactat aaaatttgct gcagcacatt ataatacaga 780
gatcttgaaa agtattgata atgagtggag aaagactcaa tgcatgccac gggaggtgtg 840
tatagatgtg gggaaggagt ttggagtcgc gacaaacacc ttctttaaac ctccatgtgt 900
gtccgtctac agatgtgggg gttgctgcaa tagtgagggg ctgcagtgca tgaacaccag 960
cacgagctac ctcagcaaga cgttatttga aattacagtg cctctctctc aaggccccaa 1020
accagtaaca atcagttttg ccaatcacac ttcctgccga tgcatgtcta aactggatgt 1080
ttacagacaa gttcattcca ttattagacg ttccctgcca gcaacactac cacagtgtca 1140
ggcagcgaac aagacctgcc ccaccaatta catgtggaat aatcacatct gcagatgcct 1200
ggctcaggaa gattttatgt tttcctcgga tgctggagat gactcaacag atggattcca 1260
tgacatctgt ggaccaaaca aggagctgga tgaagagacc tgtcagtgtg tctgcagagc 1320
ggggcttcgg cctgccagct gtggacccca caaagaacta gacagaaact catgccagtg 1380
tgtctgtaaa aacaaactct tccccagcca atgtggggcc aaccgagaat ttgatgaaaa 1440
cacatgccag tgtgtatgta aaagaacctg ccccagaaat caacccctaa atcctggaaa 1500
atgtgcctgt gaatgtacag aaagtccaca gaaatgcttg ttaaaaggaa agaagttcca 1560
ccaccaaaca tgcagctgtt acagacggcc atgtacgaac cgccagaagg cttgtgagcc 1620
aggattttca tatagtgaag aagtgtgtcg ttgtgtccct tcatattgga aaagaccaca 1680
aatgagctaa gattgtactg ttttccagtt catcgatttt ctattatgga aaactgtgtt 1740
gccacagtag aactgtctgt gaacagagag acccttgtgg gtccatgcta acaaagacaa 1800
aagtctgtct ttcctgaacc atgtggataa ctttacagaa atggactgga gctcatctgc 1860
aaaaggcctc ttgtaaagac tggttttctg ccaatgacca aacagccaag attttcctct 1920
tgtgatttct ttaaaagaat gactatataa tttatttcca ctaaaaatat tgtttctgca 1980
ttcattttta tagcaacaac aattggtaaa actcactgtg atcaatattt ttatatcatg 2040
caaaatatgt ttaaaataaa atgaaaattg tattat 2076
<210> SEQ ID NO 32
<211> LENGTH: 1822
<212> TYPE: DNA
<213> ORGANISM: Homo sapiens
<400> SEQUENCE: 32
gccgtccccg ccgccgctgc ccgccgccac cggccgcccg cccgcccggc tcctccggcc 60
gcctccgctg cgctgcgctg cgctgcctgc acccagggct cgggaggggg ccgcggagga 120
gtcgcccccc gcgcccggcc cccgcccgcc gcgcccgggc ccgcgccatg gggctctggc 180
tgtcgccgcc ccccgcgccg ccgggctagg gcgatgcggg cgcccccggc gggcggcccc 240
ggcgggcacc atgagccctc tgctccgccg cctgctgctc gccgcactcc tgcagctggc 300
ccccgcccag gcccctgtct cccagcctga tgcccctggc caccagagga aagtggtgtc 360
atggatagat gtgtatactc gcgctacctg ccagccccgg gaggtggtgg tgcccttgac 420
tgtggagctc atgggcaccg tggccaaaca gctggtgccc agctgcgtga ctgtgcagcg 480
ctgtggtggc tgctgccctg acgatggcct ggagtgtgtg cccactgggc agcaccaagt 540
ccggatgcag atcctcatga tccggtaccc gagcagtcag ctgggggaga tgtccctgga 600
agaacacagc cagtgtgaat gcagacctaa aaaaaaggac agtgctgtga agccagacag 660
ggctgccact ccccaccacc gtccccagcc ccgttctgtt ccgggctggg actctgcccc 720
cggagcaccc tccccagctg acatcaccca tcccactcca gccccaggcc cctctgccca 780
cgctgcaccc agcaccacca gcgccctgac ccccggacct gccgctgccg ctgccgacgc 840
cgcagcttcc tccgttgcca agggcggggc ttagagctca acccagacac ctgcaggtgc 900
cggaagctgc gaaggtgaca catggctttt cagactcagc agggtgactt gcctcagagg 960
ctatatccca gtgggggaac aaagaggagc ctggtaaaaa acagccaagc ccccaagacc 1020
tcagcccagg cagaagctgc tctaggacct gggcctctca gagggctctt ctgccatccc 1080
ttgtctccct gaggccatca tcaaacagga cagagttgga agaggagact gggaggcagc 1140
aagaggggtc acataccagc tcaggggaga atggagtact gtctcagttt ctaaccactc 1200
tgtgcaagta agcatcttac aactggctct tcctcccctc actaagaaga cccaaacctc 1260
tgcataatgg gatttgggct ttggtacaag aactgtgacc cccaaccctg ataaaagaga 1320
tggaaggagc tgtccctgcc tgtgtcactg tttgtcactg tccaggctgg ctggtttggg 1380
catgaatgtc tgcatcacta aatccagagc ttgtcttgct ccctcattgt gcagatggag 1440
gaaatgagga ctaaggcccc acagcagatc ccaggcaggg ccagaattat gtattcatca 1500
ctttcaagtt attgccacgc atgggagtca gggatagccc agtcaataca gactgcctgc 1560
cctcctgctc ttcaccaggg ttcttttcta gaaggagaca gccttctgtg gccagagagc 1620
ttggggtagg acccagatct actgagtgac cttgcttgtc actacccctg cctctctgag 1680
cagcagtttc cacatgtgca catagaggga acagaagatt gctgtggttg gcgtcctcgg 1740
gccccagaga agtttgagac tatctttacg taatagaaaa gaacacttgt tcttcctgcc 1800
aggcaaaaaa aaaaaaaaaa aa 1822
<210> SEQ ID NO 33
<211> LENGTH: 3936
<212> TYPE: DNA
<213> ORGANISM: Homo sapiens
<400> SEQUENCE: 33
agttttaatt gcttccaatg aggtcagcaa aggtatttat cgaaaagccc tgaataaaag 60
gctcacacac acacacaagc acacacgcgc tcacacacag agagaaaatc cttctgcctg 120
ttgatttatg gaaacaatta tgattctgct ggagaacttt tcagctgaga aatagtttgt 180
agctacagta gaaaggctca agttgcacca ggcagacaac agacatggaa ttcttatata 240
tccagctgtt agcaacaaaa caaaagtcaa atagcaaaca gcgtcacagc aactgaactt 300
actacgaact gtttttatga ggatttatca acagagttat ttaaggagga atcctgtgtt 360
gttatcagga actaaaagga taaggctaac aatttggaaa gagcaactac tctttcttaa 420
atcaatctac aattcacaga taggaagagg tcaatgacct aggagtaaca atcaactcaa 480
gattcatttt cattatgtta ttcatgaaca cccggagcac tacactataa tgcacaaatg 540
gatactgaca tggatcctgc caactttgct ctacagatca tgctttcaca ttatctgtct 600
agtgggtact atatctttag cttgcaatga catgactcca gagcaaatgg ctacaaatgt 660
gaactgttcc agccctgagc gacacacaag aagttatgat tacatggaag gaggggatat 720
aagagtgaga agactcttct gtcgaacaca gtggtacctg aggatcgata aaagaggcaa 780
agtaaaaggg acccaagaga tgaagaataa ttacaatatc atggaaatca ggacagtggc 840
agttggaatt gtggcaatca aaggggtgga aagtgaattc tatcttgcaa tgaacaagga 900
aggaaaactc tatgcaaaga aagaatgcaa tgaagattgt aacttcaaag aactaattct 960
ggaaaaccat tacaacacat atgcatcagc taaatggaca cacaacggag gggaaatgtt 1020
tgttgcctta aatcaaaagg ggattcctgt aagaggaaaa aaaacgaaga aagaacaaaa 1080
aacagcccac tttcttccta tggcaataac ttaattgcat atggtatata aagaaccagt 1140
tccagcaggg agatttcttt aagtggactg ttttctttct tctcaaaatt ttctttcctt 1200
ttatttttta gtaatcaaga aaggctggaa aactactgaa aaactgatca agctggactt 1260
gtgcatttat gtttgtttta agacactgca ttaaagaaag atttgaaaag tatacacaaa 1320
aatcagattt agtaactaaa ggttgtaaaa aattgtaaaa ctggttgtac aatcatgatg 1380
ttagtaacag taattttttt cttaaattaa tttaccctta agagtatgtt agatttgatt 1440
atctgataat gattatttaa atattcctat ctgcttataa aatggctgct ataataataa 1500
taatacagat gttgttatat aaggtatatc agacctacag gcttctggca ggatttgtca 1560
gataatcaag ccacactaac tatggaaaat gagcagcatt ttaaatgctt tctagtgaaa 1620
aattataatc tacttaaact ctaatcagaa aaaaaattct caaaaaaact attatgaaag 1680
tcaataaaat agataattta acaaaagtac aggattagaa catgcttata cctataaata 1740
agaacaaaat ttctaatgct gctcaagtgg aaagggtatt gctaaaagga tgtttccaaa 1800
aatcttgtat ataagatagc aacagtgatt gatgataata ctgtacttca tcttacttgc 1860
cacaaaataa cattttataa atcctcaaag taaaattgag aaatctttaa gtttttttca 1920
agtaacataa tctatctttg tataattcat atttgggaat atggctttta ataatgttct 1980
tcccacaaat aatcatgctt ttttcctatg gttacagcat taaactctat tttaagttgt 2040
ttttgaactt tattgttttg ttatttaagt ttatgttatt tataaaaaaa aaaccttaat 2100
aagctgtatc tgtttcatat gcttttaatt ttaaaggaat aacaaaactg tctggctcaa 2160
cggcaagttt ccctcccttt tctgactgac actaagtcta gcacacagca cttgggccag 2220
caaatcctgg aaggcagaca aaaataagag cctgaagcaa tgcttacaat agatgtctca 2280
cacagaacaa tacaaatatg taaaaaatct ttcaccacat attcttgcca attaattgga 2340
tcatataagt aaaatcatta caaatataag tatttacagg attttaaagt tagaatatat 2400
ttgaatgcat gggtagaaaa tatcatattt taaaactatg tatatttaaa tttagtaatt 2460
ttctaatctc tagaaatctc tgctgttcaa aaggtggcag cactgaaagt tgttttcctg 2520
ttagatggca agagcacaat gcccaaaata gaagatgcag ttaagaataa ggggccctga 2580
atgtcatgaa ggcttgaggt cagcctacag ataacaggat tattacaagg atgaatttcc 2640
acttcaaaag tctttcattg gcagatcttg gtagcacttt atatgttcac caatgggagg 2700
tcaatattta tctaatttaa aaggtatgct aaccactgtg gttttaattt caaaatattt 2760
gtcattcaag tccctttaca taaatagtat ttggtaatac atttatagat gagagttata 2820
tgaaaaggct aggtcaacaa aaacaataga ttcatttaat tttcctgtgg ttgacctata 2880
cgaccaggat gtagaaaact agaaagaact gcccttcctc agatatactc ttgggagaga 2940
gcatgaatgg tattctgaac tatcacctga ttcaaggact ttgctagcta ggttttgagg 3000
tcaggcttca gtaactgtag tcttgtgagc atattgaggg cagaggagga cttagttttt 3060
catatgtgtt tccttagtgc ctagcagact atctgttcat aatcagtttt cagtgtgaat 3120
tcactgaatg tttatagaca aaagaaaata cacactaaaa ctaatcttca ttttaaaagg 3180
gtaaaacatg actatacaga aatttaaata gaaatagtgt atatacatat aaaatacaag 3240
ctatgttagg accaaatgct ctttgtctat ggagttatac ttccatcaaa ttacatagca 3300
atgctgaatt aggcaaaacc aacatttagt ggtaaatcca ttcctggtag tataagtcac 3360
ctaaaaaaga cttctagaaa tatgtacttt aattatttgt ttttctccta tttttaaatt 3420
tattatgcaa attttagaaa ataaaatttg ctctagttac acacctttag aattctagaa 3480
tattaaaact gtaaggggcc tccatccctc ttactcattt gtagtctagg aaattgagat 3540
tttgatacac ctaaggtcac gcagctgggt agatatacag ctgtcacaag agtctagatc 3600
agttagcaca tgctttctac tcttcgatta ttagtattat tagctaatgg tctttggcat 3660
gtttttgttt tttatttctg ttgagatata gcctttacat ttgtacacaa atgtgactat 3720
gtcttggcaa tgcacttcat acacaatgac taatctatac tgtgatgatt tgactcaaaa 3780
ggagaaaaga aattatgtag ttttcaattc tgattcctat tcaccttttg tttatgaatg 3840
gaaagctttg tgcaaaatat acatataagc agagtaagcc ttttaaaaat gttctttgaa 3900
agataaaatt aaatacatga gtttctaaca attaga 3936
<210> SEQ ID NO 34
<211> LENGTH: 4326
<212> TYPE: DNA
<213> ORGANISM: Homo sapiens
<400> SEQUENCE: 34
gtcagctgtg ccccggtcgc cgagtggcga ggaggtgacg gtagccgcct tcctatttcc 60
gcccggcggg cagcgctgcg gggcgagtgc cagcagagag gcgctcggtc ctccctccgc 120
cctcccgcgc cgggggcagg ccctgcctag tctgcgtctt tttcccccgc accgcggcgc 180
cgctccgcca ctcgggcacc gcaggtaggg caggaggctg gagagcctgc tgcccgcccg 240
cccgtaaaat ggtcccctcg gctggacagc tcgccctgtt cgctctgggt attgtgttgg 300
ctgcgtgcca ggccttggag aacagcacgt ccccgctgag tgcagacccg cccgtggctg 360
cagcagtggt gtcccatttt aatgactgcc cagattccca cactcagttc tgcttccatg 420
gaacctgcag gtttttggtg caggaggaca agccagcatg tgtctgccat tctgggtacg 480
ttggtgcacg ctgtgagcat gcggacctcc tggccgtggt ggctgccagc cagaagaagc 540
aggccatcac cgccttggtg gtggtctcca tcgtggccct ggctgtcctt atcatcacat 600
gtgtgctgat acactgctgc caggtccgaa aacactgtga gtggtgccgg gccctcatct 660
gccggcacga gaagcccagc gccctcctga agggaagaac cgcttgctgc cactcagaaa 720
cagtggtctg aagagcccag aggaggagtt tggccaggtg gactgtggca gatcaataaa 780
gaaaggcttc ttcaggacag cactgccaga gatgcctggg tgtgccacag accttcctac 840
ttggcctgta atcacctgtg cagccttttg tgggccttca aaactctgtc aagaactccg 900
tctgcttggg gttattcagt gtgacctaga gaagaaatca gcggaccacg atttcaagac 960
ttgttaaaaa agaactgcaa agagacggac tcctgttcac ctaggtgagg tgtgtgcagc 1020
agttggtgtc tgagtccaca tgtgtgcagt tgtcttctgc cagccatgga ttccaggcta 1080
tatatttctt tttaatgggc cacctcccca caacagaatt ctgcccaaca caggagattt 1140
ctatagttat tgttttctgt catttgccta ctggggaaga aagtgaagga ggggaaactg 1200
tttaatatca catgaagacc ctagctttaa gagaagctgt atcctctaac cacgagaccc 1260
tcaaccagcc caacatcttc catggacaca tgacattgaa gaccatccca agctatcgcc 1320
acccttggag atgatgtctt atttattaga tggataatgg ttttattttt aatctcttaa 1380
gtcaatgtaa aaagtataaa accccttcag acttctacat taatgatgta tgtgttgctg 1440
actgaaaagc tatactgatt agaaatgtct ggcctcttca agacagctaa ggcttgggaa 1500
aagtcttcca gggtgcggag atggaaccag aggctgggtt actggtagga ataaaggtag 1560
gggttcagaa atggtgccat tgaagccaca aagccggtaa atgcctcaat acgttctggg 1620
agaaaactta gcaaatccat cagcagggat ctgtcccctc tgttggggag agaggaagag 1680
tgtgtgtgtc tacacaggat aaacccaata catattgtac tgctcagtga ttaaatgggt 1740
tcacttcctc gtgagccctc ggtaagtatg tttagaaata gaacattagc cacgagccat 1800
aggcatttca ggccaaatcc atgaaagggg gaccagtcat ttattttcca ttttgttgct 1860
tggttggttt gttgctttat ttttaaaagg agaagtttaa ctttgctatt tattttcgag 1920
cactaggaaa actattccag taattttttt ttcctcattt ccattcagga tgccggcttt 1980
attaacaaaa actctaacaa gtcacctcca ctatgtgggt cttcctttcc cctcaagaga 2040
aggagcaatt gttcccctga gcatctgggt ccatctgacc catggggcct gcctgtgaga 2100
aacagtgggt cccttcaaat acatagtgga tagctcatcc ctaggaattt tcattaaaat 2160
ttggaaacag agtaatgaag aaataatata taaactcctt atgtgaggaa atgctactaa 2220
tatctgaaaa gtgaaagatt tctatgtatt aactcttaag tgcacctagc ttattacatc 2280
gtgaaaggta catttaaaat atgttaaatt ggcttgaaat tttcagagaa ttttgtcttc 2340
ccctaattct tcttccttgg tctggaagaa caatttctat gaattttctc tttatttttt 2400
tttataattc agacaattct atgacccgtg tcttcatttt tggcactctt atttaacaat 2460
gccacacctg aagcacttgg atctgttcag agctgacccc ctagcaacgt agttgacaca 2520
gctccaggtt tttaaattac taaaataagt tcaagtttac atcccttggg ccagatatgt 2580
gggttgaggc ttgactgtag catcctgctt agagaccaat caacggacac tggtttttag 2640
acctctatca atcagtagtt agcatccaag agactttgca gaggcgtagg aatgaggctg 2700
gacagatggc ggaagcagag gttccctgcg aagacttgag atttagtgtc tgtgaatgtt 2760
ctagttccta ggtccagcaa gtcacacctg ccagtgccct catccttatg cctgtaacac 2820
acatgcagtg agaggcctca catatacgcc tccctagaag tgccttccaa gtcagtcctt 2880
tggaaaccag caggtctgaa aaagaggctg catcaatgca agcctggttg gaccattgtc 2940
catgcctcag gatagaacag cctggcttat ttggggattt ttcttctaga aatcaaatga 3000
ctgataagca ttggatccct ctgccattta atggcaatgg tagtctttgg ttagctgcaa 3060
aaatactcca tttcaagtta aaaatgcatc ttctaatcca tctctgcaag ctccctgtgt 3120
ttccttgccc tttagaaaat gaattgttca ctacaattag agaatcattt aacatcctga 3180
cctggtaagc tgccacacac ctggcagtgg ggagcatcgc tgtttccaat ggctcaggag 3240
acaatgaaaa gcccccattt aaaaaaataa caaacatttt ttaaaaggcc tccaatactc 3300
ttatggagcc tggatttttc ccactgctct acaggctgtg acttttttta agcatcctga 3360
caggaaatgt tttcttctac atggaaagat agacagcagc caaccctgat ctggaagaca 3420
gggccccggc tggacacacg tggaaccaag ccagggatgg gctggccatt gtgtccccgc 3480
aggagagatg ggcagaatgg ccctagagtt cttttccctg agaaaggaga aaaagatggg 3540
attgccactc acccacccac actggtaagg gaggagaatt tgtgcttctg gagcttctca 3600
agggattgtg ttttgcaggt acagaaaact gcctgttatc ttcaagccag gttttcgagg 3660
gcacatgggt caccagttgc tttttcagtc aatttggccg ggatggacta atgaggctct 3720
aacactgctc aggagacccc tgccctctag ttggttctgg gctttgatct cttccaacct 3780
gcccagtcac agaaggagga atgactcaaa tgcccaaaac caagaacaca ttgcagaagt 3840
aagacaaaca tgtatatttt taaatgttct aacataagac ctgttctctc tagccattga 3900
tttaccaggc tttctgaaag atctagtggt tcacacagag agagagagag tactgaaaaa 3960
gcaactcctc ttcttagtct taataattta ctaaaatggt caacttttca ttatctttat 4020
tataataaac ctgatgcttt tttttagaac tccttactct gatgtctgta tatgttgcac 4080
tgaaaaggtt aatatttaat gttttaattt attttgtgtg gtaagttaat tttgatttct 4140
gtaatgtgtt aatgtgatta gcagttattt tccttaatat ctgaattata cttaaagagt 4200
agtgagcaat ataagacgca attgtgtttt tcagtaatgt gcattgttat tgagttgtac 4260
tgtaccttat ttggaaggat gaaggaatga atcttttttt cctaaatcaa aaaaaaaaaa 4320
aaaaaa 4326
<210> SEQ ID NO 35
<211> LENGTH: 4323
<212> TYPE: DNA
<213> ORGANISM: Homo sapiens
<400> SEQUENCE: 35
gtcagctgtg ccccggtcgc cgagtggcga ggaggtgacg gtagccgcct tcctatttcc 60
gcccggcggg cagcgctgcg gggcgagtgc cagcagagag gcgctcggtc ctccctccgc 120
cctcccgcgc cgggggcagg ccctgcctag tctgcgtctt tttcccccgc accgcggcgc 180
cgctccgcca ctcgggcacc gcaggtaggg caggaggctg gagagcctgc tgcccgcccg 240
cccgtaaaat ggtcccctcg gctggacagc tcgccctgtt cgctctgggt attgtgttgg 300
ctgcgtgcca ggccttggag aacagcacgt ccccgctgag tgacccgccc gtggctgcag 360
cagtggtgtc ccattttaat gactgcccag attcccacac tcagttctgc ttccatggaa 420
cctgcaggtt tttggtgcag gaggacaagc cagcatgtgt ctgccattct gggtacgttg 480
gtgcacgctg tgagcatgcg gacctcctgg ccgtggtggc tgccagccag aagaagcagg 540
ccatcaccgc cttggtggtg gtctccatcg tggccctggc tgtccttatc atcacatgtg 600
tgctgataca ctgctgccag gtccgaaaac actgtgagtg gtgccgggcc ctcatctgcc 660
ggcacgagaa gcccagcgcc ctcctgaagg gaagaaccgc ttgctgccac tcagaaacag 720
tggtctgaag agcccagagg aggagtttgg ccaggtggac tgtggcagat caataaagaa 780
aggcttcttc aggacagcac tgccagagat gcctgggtgt gccacagacc ttcctacttg 840
gcctgtaatc acctgtgcag ccttttgtgg gccttcaaaa ctctgtcaag aactccgtct 900
gcttggggtt attcagtgtg acctagagaa gaaatcagcg gaccacgatt tcaagacttg 960
ttaaaaaaga actgcaaaga gacggactcc tgttcaccta ggtgaggtgt gtgcagcagt 1020
tggtgtctga gtccacatgt gtgcagttgt cttctgccag ccatggattc caggctatat 1080
atttcttttt aatgggccac ctccccacaa cagaattctg cccaacacag gagatttcta 1140
tagttattgt tttctgtcat ttgcctactg gggaagaaag tgaaggaggg gaaactgttt 1200
aatatcacat gaagacccta gctttaagag aagctgtatc ctctaaccac gagaccctca 1260
accagcccaa catcttccat ggacacatga cattgaagac catcccaagc tatcgccacc 1320
cttggagatg atgtcttatt tattagatgg ataatggttt tatttttaat ctcttaagtc 1380
aatgtaaaaa gtataaaacc ccttcagact tctacattaa tgatgtatgt gttgctgact 1440
gaaaagctat actgattaga aatgtctggc ctcttcaaga cagctaaggc ttgggaaaag 1500
tcttccaggg tgcggagatg gaaccagagg ctgggttact ggtaggaata aaggtagggg 1560
ttcagaaatg gtgccattga agccacaaag ccggtaaatg cctcaatacg ttctgggaga 1620
aaacttagca aatccatcag cagggatctg tcccctctgt tggggagaga ggaagagtgt 1680
gtgtgtctac acaggataaa cccaatacat attgtactgc tcagtgatta aatgggttca 1740
cttcctcgtg agccctcggt aagtatgttt agaaatagaa cattagccac gagccatagg 1800
catttcaggc caaatccatg aaagggggac cagtcattta ttttccattt tgttgcttgg 1860
ttggtttgtt gctttatttt taaaaggaga agtttaactt tgctatttat tttcgagcac 1920
taggaaaact attccagtaa tttttttttc ctcatttcca ttcaggatgc cggctttatt 1980
aacaaaaact ctaacaagtc acctccacta tgtgggtctt cctttcccct caagagaagg 2040
agcaattgtt cccctgagca tctgggtcca tctgacccat ggggcctgcc tgtgagaaac 2100
agtgggtccc ttcaaataca tagtggatag ctcatcccta ggaattttca ttaaaatttg 2160
gaaacagagt aatgaagaaa taatatataa actccttatg tgaggaaatg ctactaatat 2220
ctgaaaagtg aaagatttct atgtattaac tcttaagtgc acctagctta ttacatcgtg 2280
aaaggtacat ttaaaatatg ttaaattggc ttgaaatttt cagagaattt tgtcttcccc 2340
taattcttct tccttggtct ggaagaacaa tttctatgaa ttttctcttt attttttttt 2400
ataattcaga caattctatg acccgtgtct tcatttttgg cactcttatt taacaatgcc 2460
acacctgaag cacttggatc tgttcagagc tgacccccta gcaacgtagt tgacacagct 2520
ccaggttttt aaattactaa aataagttca agtttacatc ccttgggcca gatatgtggg 2580
ttgaggcttg actgtagcat cctgcttaga gaccaatcaa cggacactgg tttttagacc 2640
tctatcaatc agtagttagc atccaagaga ctttgcagag gcgtaggaat gaggctggac 2700
agatggcgga agcagaggtt ccctgcgaag acttgagatt tagtgtctgt gaatgttcta 2760
gttcctaggt ccagcaagtc acacctgcca gtgccctcat ccttatgcct gtaacacaca 2820
tgcagtgaga ggcctcacat atacgcctcc ctagaagtgc cttccaagtc agtcctttgg 2880
aaaccagcag gtctgaaaaa gaggctgcat caatgcaagc ctggttggac cattgtccat 2940
gcctcaggat agaacagcct ggcttatttg gggatttttc ttctagaaat caaatgactg 3000
ataagcattg gatccctctg ccatttaatg gcaatggtag tctttggtta gctgcaaaaa 3060
tactccattt caagttaaaa atgcatcttc taatccatct ctgcaagctc cctgtgtttc 3120
cttgcccttt agaaaatgaa ttgttcacta caattagaga atcatttaac atcctgacct 3180
ggtaagctgc cacacacctg gcagtgggga gcatcgctgt ttccaatggc tcaggagaca 3240
atgaaaagcc cccatttaaa aaaataacaa acatttttta aaaggcctcc aatactctta 3300
tggagcctgg atttttccca ctgctctaca ggctgtgact ttttttaagc atcctgacag 3360
gaaatgtttt cttctacatg gaaagataga cagcagccaa ccctgatctg gaagacaggg 3420
ccccggctgg acacacgtgg aaccaagcca gggatgggct ggccattgtg tccccgcagg 3480
agagatgggc agaatggccc tagagttctt ttccctgaga aaggagaaaa agatgggatt 3540
gccactcacc cacccacact ggtaagggag gagaatttgt gcttctggag cttctcaagg 3600
gattgtgttt tgcaggtaca gaaaactgcc tgttatcttc aagccaggtt ttcgagggca 3660
catgggtcac cagttgcttt ttcagtcaat ttggccggga tggactaatg aggctctaac 3720
actgctcagg agacccctgc cctctagttg gttctgggct ttgatctctt ccaacctgcc 3780
cagtcacaga aggaggaatg actcaaatgc ccaaaaccaa gaacacattg cagaagtaag 3840
acaaacatgt atatttttaa atgttctaac ataagacctg ttctctctag ccattgattt 3900
accaggcttt ctgaaagatc tagtggttca cacagagaga gagagagtac tgaaaaagca 3960
actcctcttc ttagtcttaa taatttacta aaatggtcaa cttttcatta tctttattat 4020
aataaacctg atgctttttt ttagaactcc ttactctgat gtctgtatat gttgcactga 4080
aaaggttaat atttaatgtt ttaatttatt ttgtgtggta agttaatttt gatttctgta 4140
atgtgttaat gtgattagca gttattttcc ttaatatctg aattatactt aaagagtagt 4200
gagcaatata agacgcaatt gtgtttttca gtaatgtgca ttgttattga gttgtactgt 4260
accttatttg gaaggatgaa ggaatgaatc tttttttcct aaatcaaaaa aaaaaaaaaa 4320
aaa 4323
<210> SEQ ID NO 36
<211> LENGTH: 2217
<212> TYPE: DNA
<213> ORGANISM: Homo sapiens
<400> SEQUENCE: 36
ccccgccgcc gccgcccttc gcgccctggg ccatctccct cccacctccc tccgcggagc 60
agccagacag cgagggcccc ggccgggggc aggggggacg ccccgtccgg ggcacccccc 120
cggctctgag ccgcccgcgg ggccggcctc ggcccggagc ggaggaagga gtcgccgagg 180
agcagcctga ggccccagag tctgagacga gccgccgccg cccccgccac tgcggggagg 240
agggggagga ggagcgggag gagggacgag ctggtcggga gaagaggaaa aaaacttttg 300
agacttttcc gttgccgctg ggagccggag gcgcggggac ctcttggcgc gacgctgccc 360
cgcgaggagg caggacttgg ggaccccaga ccgcctccct ttgccgccgg ggacgcttgc 420
tccctccctg ccccctacac ggcgtccctc aggcgccccc attccggacc agccctcggg 480
agtcgccgac ccggcctccc gcaaagactt ttccccagac ctcgggcgca ccccctgcac 540
gccgccttca tccccggcct gtctcctgag cccccgcgca tcctagaccc tttctcctcc 600
aggagacgga tctctctccg acctgccaca gatcccctat tcaagaccac ccaccttctg 660
gtaccagatc gcgcccatct aggttatttc cgtgggatac tgagacaccc ccggtccaag 720
cctcccctcc accactgcgc ccttctccct gaggacctca gctttccctc gaggccctcc 780
taccttttgc cgggagaccc ccagcccctg caggggcggg gcctccccac cacaccagcc 840
ctgttcgcgc tctcggcagt gccggggggc gccgcctccc ccatgccgcc ctccgggctg 900
cggctgctgc cgctgctgct accgctgctg tggctactgg tgctgacgcc tggccggccg 960
gccgcgggac tatccacctg caagactatc gacatggagc tggtgaagcg gaagcgcatc 1020
gaggccatcc gcggccagat cctgtccaag ctgcggctcg ccagcccccc gagccagggg 1080
gaggtgccgc ccggcccgct gcccgaggcc gtgctcgccc tgtacaacag cacccgcgac 1140
cgggtggccg gggagagtgc agaaccggag cccgagcctg aggccgacta ctacgccaag 1200
gaggtcaccc gcgtgctaat ggtggaaacc cacaacgaaa tctatgacaa gttcaagcag 1260
agtacacaca gcatatatat gttcttcaac acatcagagc tccgagaagc ggtacctgaa 1320
cccgtgttgc tctcccgggc agagctgcgt ctgctgaggc tcaagttaaa agtggagcag 1380
cacgtggagc tgtaccagaa atacagcaac aattcctggc gatacctcag caaccggctg 1440
ctggcaccca gcgactcgcc agagtggtta tcttttgatg tcaccggagt tgtgcggcag 1500
tggttgagcc gtggagggga aattgagggc tttcgcctta gcgcccactg ctcctgtgac 1560
agcagggata acacactgca agtggacatc aacgggttca ctaccggccg ccgaggtgac 1620
ctggccacca ttcatggcat gaaccggcct ttcctgcttc tcatggccac cccgctggag 1680
agggcccagc atctgcaaag ctcccggcac cgccgagccc tggacaccaa ctattgcttc 1740
agctccacgg agaagaactg ctgcgtgcgg cagctgtaca ttgacttccg caaggacctc 1800
ggctggaagt ggatccacga gcccaagggc taccatgcca acttctgcct cgggccctgc 1860
ccctacattt ggagcctgga cacgcagtac agcaaggtcc tggccctgta caaccagcat 1920
aacccgggcg cctcggcggc gccgtgctgc gtgccgcagg cgctggagcc gctgcccatc 1980
gtgtactacg tgggccgcaa gcccaaggtg gagcagctgt ccaacatgat cgtgcgctcc 2040
tgcaagtgca gctgaggtcc cgccccgccc cgccccgccc cggcaggccc ggccccaccc 2100
cgccccgccc ccgctgcctt gcccatgggg gctgtattta aggacacccg tgccccaagc 2160
ccacctgggg ccccattaaa gatggagaga ggactgcgga aaaaaaaaaa aaaaaaa 2217
<210> SEQ ID NO 37
<211> LENGTH: 5966
<212> TYPE: DNA
<213> ORGANISM: Homo sapiens
<400> SEQUENCE: 37
gtgatgttat ctgctggcag cagaaggttc gctccgagcg gagctccaga agctcctgac 60
aagagaaaga cagattgaga tagagataga aagagaaaga gagaaagaga cagcagagcg 120
agagcgcaag tgaaagaggc aggggagggg gatggagaat attagcctga cggtctaggg 180
agtcatccag gaacaaactg aggggctgcc cggctgcaga caggaggaga cagagaggat 240
ctattttagg gtggcaagtg cctacctacc ctaagcgagc aattccacgt tggggagaag 300
ccagcagagg ttgggaaagg gtgggagtcc aagggagccc ctgcgcaacc ccctcaggaa 360
taaaactccc cagccagggt gtcgcaaggg ctgccgttgt gatccgcagg gggtgaacgc 420
aaccgcgacg gctgatcgtc tgtggctggg ttggcgtttg gagcaagaga aggaggagca 480
ggagaaggag ggagctggag gctggaagcg tttgcaagcg gcggcggcag caacgtggag 540
taaccaagcg ggtcagcgcg cgcccgccag ggtgtaggcc acggagcgca gctcccagag 600
caggatccgc gccgcctcag cagcctctgc ggcccctgcg gcacccgacc gagtaccgag 660
cgccctgcga agcgcaccct cctccccgcg gtgcgctggg ctcgccccca gcgcgcgcac 720
acgcacacac acacacacac acacacacgc acgcacacac gtgtgcgctt ctctgctccg 780
gagctgctgc tgctcctgct ctcagcgccg cagtggaagg caggaccgaa ccgctccttc 840
tttaaatata taaatttcag cccaggtcag cctcggcggc ccccctcacc gcgctcccgg 900
cgcccctccc gtcagttcgc cagctgccag ccccgggacc ttttcatctc ttcccttttg 960
gccggaggag ccgagttcag atccgccact ccgcacccga gactgacaca ctgaactcca 1020
cttcctcctc ttaaatttat ttctacttaa tagccactcg tctctttttt tccccatctc 1080
attgctccaa gaattttttt cttcttactc gccaaagtca gggttccctc tgcccgtccc 1140
gtattaatat ttccactttt ggaactactg gccttttctt tttaaaggaa ttcaagcagg 1200
atacgttttt ctgttgggca ttgactagat tgtttgcaaa agtttcgcat caaaaacaac 1260
aacaacaaaa aaccaaacaa ctctccttga tctatacttt gagaattgtt gatttctttt 1320
ttttattctg acttttaaaa acaacttttt tttccacttt tttaaaaaat gcactactgt 1380
gtgctgagcg cttttctgat cctgcatctg gtcacggtcg cgctcagcct gtctacctgc 1440
agcacactcg atatggacca gttcatgcgc aagaggatcg aggcgatccg cgggcagatc 1500
ctgagcaagc tgaagctcac cagtccccca gaagactatc ctgagcccga ggaagtcccc 1560
ccggaggtga tttccatcta caacagcacc agggacttgc tccaggagaa ggcgagccgg 1620
agggcggccg cctgcgagcg cgagaggagc gacgaagagt actacgccaa ggaggtttac 1680
aaaatagaca tgccgccctt cttcccctcc gaaactgtct gcccagttgt tacaacaccc 1740
tctggctcag tgggcagctt gtgctccaga cagtcccagg tgctctgtgg gtaccttgat 1800
gccatcccgc ccactttcta cagaccctac ttcagaattg ttcgatttga cgtctcagca 1860
atggagaaga atgcttccaa tttggtgaaa gcagagttca gagtctttcg tttgcagaac 1920
ccaaaagcca gagtgcctga acaacggatt gagctatatc agattctcaa gtccaaagat 1980
ttaacatctc caacccagcg ctacatcgac agcaaagttg tgaaaacaag agcagaaggc 2040
gaatggctct ccttcgatgt aactgatgct gttcatgaat ggcttcacca taaagacagg 2100
aacctgggat ttaaaataag cttacactgt ccctgctgca cttttgtacc atctaataat 2160
tacatcatcc caaataaaag tgaagaacta gaagcaagat ttgcaggtat tgatggcacc 2220
tccacatata ccagtggtga tcagaaaact ataaagtcca ctaggaaaaa aaacagtggg 2280
aagaccccac atctcctgct aatgttattg ccctcctaca gacttgagtc acaacagacc 2340
aaccggcgga agaagcgtgc tttggatgcg gcctattgct ttagaaatgt gcaggataat 2400
tgctgcctac gtccacttta cattgatttc aagagggatc tagggtggaa atggatacac 2460
gaacccaaag ggtacaatgc caacttctgt gctggagcat gcccgtattt atggagttca 2520
gacactcagc acagcagggt cctgagctta tataatacca taaatccaga agcatctgct 2580
tctccttgct gcgtgtccca agatttagaa cctctaacca ttctctacta cattggcaaa 2640
acacccaaga ttgaacagct ttctaatatg attgtaaagt cttgcaaatg cagctaaaat 2700
tcttggaaaa gtggcaagac caaaatgaca atgatgatga taatgatgat gacgacgaca 2760
acgatgatgc ttgtaacaag aaaacataag agagccttgg ttcatcagtg ttaaaaaatt 2820
tttgaaaagg cggtactagt tcagacactt tggaagtttg tgttctgttt gttaaaactg 2880
gcatctgaca caaaaaaagt tgaaggcctt attctacatt tcacctactt tgtaagtgag 2940
agagacaaga agcaaatttt ttttaaagaa aaaaataaac actggaagaa tttattagtg 3000
ttaattatgt gaacaacgac aacaacaaca acaacaacaa acaggaaaat cccattaagt 3060
ggagttgctg tacgtaccgt tcctatcccg cgcctcactt gatttttctg tattgctatg 3120
caataggcac ccttcccatt cttactctta gagttaacag tgagttattt attgtgtgtt 3180
actatataat gaacgtttca ttgcccttgg aaaataaaac aggtgtataa agtggagacc 3240
aaatactttg ccagaaactc atggatggct taaggaactt gaactcaaac gagccagaaa 3300
aaaagaggtc atattaatgg gatgaaaacc caagtgagtt attatatgac cgagaaagtc 3360
tgcattaaga taaagaccct gaaaacacat gttatgtatc agctgcctaa ggaagcttct 3420
tgtaaggtcc aaaaactaaa aagactgtta ataaaagaaa ctttcagtca gaataagtct 3480
gtaagttttt ttttttcttt ttaattgtaa atggttcttt gtcagtttag taaaccagtg 3540
aaatgttgaa atgttttgac atgtactggt caaacttcag accttaaaat attgctgtat 3600
agctatgcta taggtttttt cctttgtttt ggtatatgta accataccta tattattaaa 3660
atagatggat atagaagcca gcataattga aaacacatct gcagatctct tttgcaaact 3720
attaaatcaa aacattaact actttatgtg taatgtgtaa atttttacca tattttttat 3780
attctgtaat aatgtcaact atgatttaga ttgacttaaa tttgggctct ttttaatgat 3840
cactcacaaa tgtatgtttc ttttagctgg ccagtacttt tgagtaaagc ccctatagtt 3900
tgacttgcac tacaaatgca tttttttttt aataacattt gccctacttg tgctttgtgt 3960
ttctttcatt attatgacat aagctacctg ggtccacttg tcttttcttt tttttgtttc 4020
acagaaaaga tgggttcgag ttcagtggtc ttcatcttcc aagcatcatt actaaccaag 4080
tcagacgtta acaaattttt atgttaggaa aaggaggaat gttatagata catagaaaat 4140
tgaagtaaaa tgttttcatt ttagcaagga tttagggttc taactaaaac tcagaatctt 4200
tattgagtta agaaaagttt ctctaccttg gtttaatcaa tatttttgta aaatcctatt 4260
gttattacaa agaggacact tcataggaaa catctttttc tttagtcagg tttttaatat 4320
tcagggggaa attgaaagat atatatttta gtcgattttt caaaagggga aaaaagtcca 4380
ggtcagcata agtcattttg tgtatttcac tgaagttata aggtttttat aaatgttctt 4440
tgaaggggaa aaggcacaag ccaatttttc ctatgatcaa aaaattcttt ctttcctctg 4500
agtgagagtt atctatatct gaggctaaag tttaccttgc tttaataaat aatttgccac 4560
atcattgcag aagaggtatc ctcatgctgg ggttaataga atatgtcagt ttatcacttg 4620
tcgcttattt agctttaaaa taaaaattaa taggcaaagc aatggaatat ttgcagtttc 4680
acctaaagag cagcataagg aggcgggaat ccaaagtgaa gttgtttgat atggtctact 4740
tcttttttgg aatttcctga ccattaatta aagaattgga tttgcaagtt tgaaaactgg 4800
aaaagcaaga gatgggatgc cataatagta aacagccctt gtgttggatg taacccaatc 4860
ccagatttga gtgtgtgttg attatttttt tgtcttccac ttttctatta tgtgtaaatc 4920
acttttattt ctgcagacat tttcctctca gataggatga cattttgttt tgtattattt 4980
tgtctttcct catgaatgca ctgataatat tttaaatgct ctattttaag atctcttgaa 5040
tctgtttttt ttttttttaa tttgggggtt ctgtaaggtc tttatttccc ataagtaaat 5100
attgccatgg gaggggggtg gaggtggcaa ggaaggggtg aagtgctagt atgcaagtgg 5160
gcagcaatta tttttgtgtt aatcagcagt acaatttgat cgttggcatg gttaaaaaat 5220
ggaatataag attagctgtt ttgtattttg atgaccaatt acgctgtatt ttaacacgat 5280
gtatgtctgt ttttgtggtg ctctagtggt aaataaatta tttcgatgat atgtggatgt 5340
ctttttccta tcagtaccat catcgagtct agaaaacacc tgtgatgcaa taagactatc 5400
tcaagctgga aaagtcatac cacctttccg attgccctct gtgctttctc ccttaaggac 5460
agtcacttca gaagtcatgc tttaaagcac aagagtcagg ccatatccat caaggataga 5520
agaaatccct gtgccgtctt tttattccct tatttattgc tatttggtaa ttgtttgaga 5580
tttagtttcc atccagcttg actgccgacc agaaaaaatg cagagagatg tttgcaccat 5640
gctttggctt tctggttcta tgttctgcca acgccagggc caaaagaact ggtctagaca 5700
gtatcccctg tagccccata acttggatag ttgctgagcc agccagatat aacaagagcc 5760
acgtgctttc tggggttggt tgtttgggat cagctacttg cctgtcagtt tcactggtac 5820
cactgcacca caaacaaaaa aacccaccct atttcctcca atttttttgg ctgctaccta 5880
caagaccaga ctcctcaaac gagttgccaa tctcttaata aataggatta ataaaaaaag 5940
taattgtgac tcaaaaaaaa aaaaaa 5966
<210> SEQ ID NO 38
<211> LENGTH: 5882
<212> TYPE: DNA
<213> ORGANISM: Homo sapiens
<400> SEQUENCE: 38
gtgatgttat ctgctggcag cagaaggttc gctccgagcg gagctccaga agctcctgac 60
aagagaaaga cagattgaga tagagataga aagagaaaga gagaaagaga cagcagagcg 120
agagcgcaag tgaaagaggc aggggagggg gatggagaat attagcctga cggtctaggg 180
agtcatccag gaacaaactg aggggctgcc cggctgcaga caggaggaga cagagaggat 240
ctattttagg gtggcaagtg cctacctacc ctaagcgagc aattccacgt tggggagaag 300
ccagcagagg ttgggaaagg gtgggagtcc aagggagccc ctgcgcaacc ccctcaggaa 360
taaaactccc cagccagggt gtcgcaaggg ctgccgttgt gatccgcagg gggtgaacgc 420
aaccgcgacg gctgatcgtc tgtggctggg ttggcgtttg gagcaagaga aggaggagca 480
ggagaaggag ggagctggag gctggaagcg tttgcaagcg gcggcggcag caacgtggag 540
taaccaagcg ggtcagcgcg cgcccgccag ggtgtaggcc acggagcgca gctcccagag 600
caggatccgc gccgcctcag cagcctctgc ggcccctgcg gcacccgacc gagtaccgag 660
cgccctgcga agcgcaccct cctccccgcg gtgcgctggg ctcgccccca gcgcgcgcac 720
acgcacacac acacacacac acacacacgc acgcacacac gtgtgcgctt ctctgctccg 780
gagctgctgc tgctcctgct ctcagcgccg cagtggaagg caggaccgaa ccgctccttc 840
tttaaatata taaatttcag cccaggtcag cctcggcggc ccccctcacc gcgctcccgg 900
cgcccctccc gtcagttcgc cagctgccag ccccgggacc ttttcatctc ttcccttttg 960
gccggaggag ccgagttcag atccgccact ccgcacccga gactgacaca ctgaactcca 1020
cttcctcctc ttaaatttat ttctacttaa tagccactcg tctctttttt tccccatctc 1080
attgctccaa gaattttttt cttcttactc gccaaagtca gggttccctc tgcccgtccc 1140
gtattaatat ttccactttt ggaactactg gccttttctt tttaaaggaa ttcaagcagg 1200
atacgttttt ctgttgggca ttgactagat tgtttgcaaa agtttcgcat caaaaacaac 1260
aacaacaaaa aaccaaacaa ctctccttga tctatacttt gagaattgtt gatttctttt 1320
ttttattctg acttttaaaa acaacttttt tttccacttt tttaaaaaat gcactactgt 1380
gtgctgagcg cttttctgat cctgcatctg gtcacggtcg cgctcagcct gtctacctgc 1440
agcacactcg atatggacca gttcatgcgc aagaggatcg aggcgatccg cgggcagatc 1500
ctgagcaagc tgaagctcac cagtccccca gaagactatc ctgagcccga ggaagtcccc 1560
ccggaggtga tttccatcta caacagcacc agggacttgc tccaggagaa ggcgagccgg 1620
agggcggccg cctgcgagcg cgagaggagc gacgaagagt actacgccaa ggaggtttac 1680
aaaatagaca tgccgccctt cttcccctcc gaaaatgcca tcccgcccac tttctacaga 1740
ccctacttca gaattgttcg atttgacgtc tcagcaatgg agaagaatgc ttccaatttg 1800
gtgaaagcag agttcagagt ctttcgtttg cagaacccaa aagccagagt gcctgaacaa 1860
cggattgagc tatatcagat tctcaagtcc aaagatttaa catctccaac ccagcgctac 1920
atcgacagca aagttgtgaa aacaagagca gaaggcgaat ggctctcctt cgatgtaact 1980
gatgctgttc atgaatggct tcaccataaa gacaggaacc tgggatttaa aataagctta 2040
cactgtccct gctgcacttt tgtaccatct aataattaca tcatcccaaa taaaagtgaa 2100
gaactagaag caagatttgc aggtattgat ggcacctcca catataccag tggtgatcag 2160
aaaactataa agtccactag gaaaaaaaac agtgggaaga ccccacatct cctgctaatg 2220
ttattgccct cctacagact tgagtcacaa cagaccaacc ggcggaagaa gcgtgctttg 2280
gatgcggcct attgctttag aaatgtgcag gataattgct gcctacgtcc actttacatt 2340
gatttcaaga gggatctagg gtggaaatgg atacacgaac ccaaagggta caatgccaac 2400
ttctgtgctg gagcatgccc gtatttatgg agttcagaca ctcagcacag cagggtcctg 2460
agcttatata ataccataaa tccagaagca tctgcttctc cttgctgcgt gtcccaagat 2520
ttagaacctc taaccattct ctactacatt ggcaaaacac ccaagattga acagctttct 2580
aatatgattg taaagtcttg caaatgcagc taaaattctt ggaaaagtgg caagaccaaa 2640
atgacaatga tgatgataat gatgatgacg acgacaacga tgatgcttgt aacaagaaaa 2700
cataagagag ccttggttca tcagtgttaa aaaatttttg aaaaggcggt actagttcag 2760
acactttgga agtttgtgtt ctgtttgtta aaactggcat ctgacacaaa aaaagttgaa 2820
ggccttattc tacatttcac ctactttgta agtgagagag acaagaagca aatttttttt 2880
aaagaaaaaa ataaacactg gaagaattta ttagtgttaa ttatgtgaac aacgacaaca 2940
acaacaacaa caacaaacag gaaaatccca ttaagtggag ttgctgtacg taccgttcct 3000
atcccgcgcc tcacttgatt tttctgtatt gctatgcaat aggcaccctt cccattctta 3060
ctcttagagt taacagtgag ttatttattg tgtgttacta tataatgaac gtttcattgc 3120
ccttggaaaa taaaacaggt gtataaagtg gagaccaaat actttgccag aaactcatgg 3180
atggcttaag gaacttgaac tcaaacgagc cagaaaaaaa gaggtcatat taatgggatg 3240
aaaacccaag tgagttatta tatgaccgag aaagtctgca ttaagataaa gaccctgaaa 3300
acacatgtta tgtatcagct gcctaaggaa gcttcttgta aggtccaaaa actaaaaaga 3360
ctgttaataa aagaaacttt cagtcagaat aagtctgtaa gttttttttt ttctttttaa 3420
ttgtaaatgg ttctttgtca gtttagtaaa ccagtgaaat gttgaaatgt tttgacatgt 3480
actggtcaaa cttcagacct taaaatattg ctgtatagct atgctatagg ttttttcctt 3540
tgttttggta tatgtaacca tacctatatt attaaaatag atggatatag aagccagcat 3600
aattgaaaac acatctgcag atctcttttg caaactatta aatcaaaaca ttaactactt 3660
tatgtgtaat gtgtaaattt ttaccatatt ttttatattc tgtaataatg tcaactatga 3720
tttagattga cttaaatttg ggctcttttt aatgatcact cacaaatgta tgtttctttt 3780
agctggccag tacttttgag taaagcccct atagtttgac ttgcactaca aatgcatttt 3840
ttttttaata acatttgccc tacttgtgct ttgtgtttct ttcattatta tgacataagc 3900
tacctgggtc cacttgtctt ttcttttttt tgtttcacag aaaagatggg ttcgagttca 3960
gtggtcttca tcttccaagc atcattacta accaagtcag acgttaacaa atttttatgt 4020
taggaaaagg aggaatgtta tagatacata gaaaattgaa gtaaaatgtt ttcattttag 4080
caaggattta gggttctaac taaaactcag aatctttatt gagttaagaa aagtttctct 4140
accttggttt aatcaatatt tttgtaaaat cctattgtta ttacaaagag gacacttcat 4200
aggaaacatc tttttcttta gtcaggtttt taatattcag ggggaaattg aaagatatat 4260
attttagtcg atttttcaaa aggggaaaaa agtccaggtc agcataagtc attttgtgta 4320
tttcactgaa gttataaggt ttttataaat gttctttgaa ggggaaaagg cacaagccaa 4380
tttttcctat gatcaaaaaa ttctttcttt cctctgagtg agagttatct atatctgagg 4440
ctaaagttta ccttgcttta ataaataatt tgccacatca ttgcagaaga ggtatcctca 4500
tgctggggtt aatagaatat gtcagtttat cacttgtcgc ttatttagct ttaaaataaa 4560
aattaatagg caaagcaatg gaatatttgc agtttcacct aaagagcagc ataaggaggc 4620
gggaatccaa agtgaagttg tttgatatgg tctacttctt ttttggaatt tcctgaccat 4680
taattaaaga attggatttg caagtttgaa aactggaaaa gcaagagatg ggatgccata 4740
atagtaaaca gcccttgtgt tggatgtaac ccaatcccag atttgagtgt gtgttgatta 4800
tttttttgtc ttccactttt ctattatgtg taaatcactt ttatttctgc agacattttc 4860
ctctcagata ggatgacatt ttgttttgta ttattttgtc tttcctcatg aatgcactga 4920
taatatttta aatgctctat tttaagatct cttgaatctg tttttttttt ttttaatttg 4980
ggggttctgt aaggtcttta tttcccataa gtaaatattg ccatgggagg ggggtggagg 5040
tggcaaggaa ggggtgaagt gctagtatgc aagtgggcag caattatttt tgtgttaatc 5100
agcagtacaa tttgatcgtt ggcatggtta aaaaatggaa tataagatta gctgttttgt 5160
attttgatga ccaattacgc tgtattttaa cacgatgtat gtctgttttt gtggtgctct 5220
agtggtaaat aaattatttc gatgatatgt ggatgtcttt ttcctatcag taccatcatc 5280
gagtctagaa aacacctgtg atgcaataag actatctcaa gctggaaaag tcataccacc 5340
tttccgattg ccctctgtgc tttctccctt aaggacagtc acttcagaag tcatgcttta 5400
aagcacaaga gtcaggccat atccatcaag gatagaagaa atccctgtgc cgtcttttta 5460
ttcccttatt tattgctatt tggtaattgt ttgagattta gtttccatcc agcttgactg 5520
ccgaccagaa aaaatgcaga gagatgtttg caccatgctt tggctttctg gttctatgtt 5580
ctgccaacgc cagggccaaa agaactggtc tagacagtat cccctgtagc cccataactt 5640
ggatagttgc tgagccagcc agatataaca agagccacgt gctttctggg gttggttgtt 5700
tgggatcagc tacttgcctg tcagtttcac tggtaccact gcaccacaaa caaaaaaacc 5760
caccctattt cctccaattt ttttggctgc tacctacaag accagactcc tcaaacgagt 5820
tgccaatctc ttaataaata ggattaataa aaaaagtaat tgtgactcaa aaaaaaaaaa 5880
aa 5882
<210> SEQ ID NO 39
<211> LENGTH: 3183
<212> TYPE: DNA
<213> ORGANISM: Homo sapiens
<400> SEQUENCE: 39
gacagaagca atggccgagg cagaagacaa gccgaggtgc tggtgaccct gggcgtctga 60
gtggatgatt ggggctgctg cgctcagagg cctgcctccc tgccttccaa tgcatataac 120
cccacacccc agccaatgaa gacgagaggc agcgtgaaca aagtcattta gaaagccccc 180
gaggaagtgt aaacaaaaga gaaagcatga atggagtgcc tgagagacaa gtgtgtcctg 240
tactgccccc acctttagct gggccagcaa ctgcccggcc ctgcttctcc ccacctactc 300
actggtgatc tttttttttt tacttttttt tcccttttct tttccattct cttttcttat 360
tttctttcaa ggcaaggcaa ggattttgat tttgggaccc agccatggtc cttctgcttc 420
ttctttaaaa tacccacttt ctccccatcg ccaagcggcg tttggcaata tcagatatcc 480
actctattta tttttaccta aggaaaaact ccagctccct tcccactccc agctgccttg 540
ccacccctcc cagccctctg cttgccctcc acctggcctg ctgggagtca gagcccagca 600
aaacctgttt agacacatgg acaagaatcc cagcgctaca aggcacacag tccgcttctt 660
cgtcctcagg gttgccagcg cttcctggaa gtcctgaagc tctcgcagtg cagtgagttc 720
atgcaccttc ttgccaagcc tcagtctttg ggatctgggg aggccgcctg gttttcctcc 780
ctccttctgc acgtctgctg gggtctcttc ctctccaggc cttgccgtcc ccctggcctc 840
tcttcccagc tcacacatga agatgcactt gcaaagggct ctggtggtcc tggccctgct 900
gaactttgcc acggtcagcc tctctctgtc cacttgcacc accttggact tcggccacat 960
caagaagaag agggtggaag ccattagggg acagatcttg agcaagctca ggctcaccag 1020
cccccctgag ccaacggtga tgacccacgt cccctatcag gtcctggccc tttacaacag 1080
cacccgggag ctgctggagg agatgcatgg ggagagggag gaaggctgca cccaggaaaa 1140
caccgagtcg gaatactatg ccaaagaaat ccataaattc gacatgatcc aggggctggc 1200
ggagcacaac gaactggctg tctgccctaa aggaattacc tccaaggttt tccgcttcaa 1260
tgtgtcctca gtggagaaaa atagaaccaa cctattccga gcagaattcc gggtcttgcg 1320
ggtgcccaac cccagctcta agcggaatga gcagaggatc gagctcttcc agatccttcg 1380
gccagatgag cacattgcca aacagcgcta tatcggtggc aagaatctgc ccacacgggg 1440
cactgccgag tggctgtcct ttgatgtcac tgacactgtg cgtgagtggc tgttgagaag 1500
agagtccaac ttaggtctag aaatcagcat tcactgtcca tgtcacacct ttcagcccaa 1560
tggagatatc ctggaaaaca ttcacgaggt gatggaaatc aaattcaaag gcgtggacaa 1620
tgaggatgac catggccgtg gagatctggg gcgcctcaag aagcagaagg atcaccacaa 1680
ccctcatcta atcctcatga tgattccccc acaccggctc gacaacccgg gccagggggg 1740
tcagaggaag aagcgggctt tggacaccaa ttactgcttc cgcaacttgg aggagaactg 1800
ctgtgtgcgc cccctctaca ttgacttccg acaggatctg ggctggaagt gggtccatga 1860
acctaagggc tactatgcca acttctgctc aggcccttgc ccatacctcc gcagtgcaga 1920
cacaacccac agcacggtgc tgggactgta caacactctg aaccctgaag catctgcctc 1980
gccttgctgc gtgccccagg acctggagcc cctgaccatc ctgtactatg ttgggaggac 2040
ccccaaagtg gagcagctct ccaacatggt ggtgaagtct tgtaaatgta gctgagaccc 2100
cacgtgcgac agagagaggg gagagagaac caccactgcc tgactgcccg ctcctcggga 2160
aacacacaag caacaaacct cactgagagg cctggagccc acaaccttcg gctccgggca 2220
aatggctgag atggaggttt ccttttggaa catttctttc ttgctggctc tgagaatcac 2280
ggtggtaaag aaagtgtggg tttggttaga ggaaggctga actcttcaga acacacagac 2340
tttctgtgac gcagacagag gggatgggga tagaggaaag ggatggtaag ttgagatgtt 2400
gtgtggcaat gggatttggg ctaccctaaa gggagaagga agggcagaga atggctgggt 2460
cagggccaga ctggaagaca cttcagatct gaggttggat ttgctcattg ctgtaccaca 2520
tctgctctag ggaatctgga ttatgttata caaggcaagc attttttttt tttttttaaa 2580
gacaggttac gaagacaaag tcccagaatt gtatctcata ctgtctggga ttaagggcaa 2640
atctattact tttgcaaact gtcctctaca tcaattaaca tcgtgggtca ctacagggag 2700
aaaatccagg tcatgcagtt cctggcccat caactgtatt gggccttttg gatatgctga 2760
acgcagaaga aagggtggaa atcaaccctc tcctgtctgc cctctgggtc cctcctctca 2820
cctctccctc gatcatattt ccccttggac acttggttag acgccttcca ggtcaggatg 2880
cacatttctg gattgtggtt ccatgcagcc ttggggcatt atgggttctt cccccacttc 2940
ccctccaaga ccctgtgttc atttggtgtt cctggaagca ggtgctacaa catgtgaggc 3000
attcggggaa gctgcacatg tgccacacag tgacttggcc ccagacgcat agactgaggt 3060
ataaagacaa gtatgaatat tactctcaaa atctttgtat aaataaatat ttttggggca 3120
tcctggatga tttcatcttc tggaatattg tttctagaac agtaaaagcc ttattctaag 3180
gtg 3183
<210> SEQ ID NO 40
<211> LENGTH: 4162
<212> TYPE: DNA
<213> ORGANISM: Homo sapiens
<400> SEQUENCE: 40
agaagtccat tcggctcaca catttgcccc aagacaaacc acgttaaaat aacacccagg 60
gtagctgctg ccaccgtctt ctgtctctac ctccctcctg gctggccaat ggctctgtgt 120
tcctgggcct gctgctggct gtccagagta ggggttgctt agagctgtgt gcatccctgc 180
gggtggtgtg ggagtgggcg gttgtctaaa ggcaggtccc ctctactgat aaacaaggac 240
cggagataga cctagaggct gacattcttg gctcccccag cctacacccc ccccacctcg 300
atttcccaca gagccctagg gacgggtagc cagctctgtg gcatggtatc tggaggcagg 360
ccagcaacct gatgtgcatg ccacggcccg tccctctccc cactcagagc tgcagtagcc 420
tggaggttca gagagccggg ctactctgag aagaagacac caagtggatt ctgcttcccc 480
tgggacagca ctgagcgagt gtggagagag gtacagccct cggcctacaa gctctttagt 540
cttgaaagcg ccacaagcag cagctgctga gccatggctg aaggggaaat caccaccttc 600
acagccctga ccgagaagtt taatctgcct ccagggaatt acaagaagcc caaactcctc 660
tactgtagca acgggggcca cttcctgagg atccttccgg atggcacagt ggatgggaca 720
agggacagga gcgaccagca cattcagctg cagctcagtg cggaaagcgt gggggaggtg 780
tatataaaga gtaccgagac tggccagtac ttggccatgg acaccgacgg gcttttatac 840
ggctcacaga caccaaatga ggaatgtttg ttcctggaaa ggctggagga gaaccattac 900
aacacctata tatccaagaa gcatgcagag aagaattggt ttgttggcct caagaagaat 960
gggagctgca aacgcggtcc tcggactcac tatggccaga aagcaatctt gtttctcccc 1020
ctgccagtct cttctgatta aagagatctg ttctgggtgt tgaccactcc agagaagttt 1080
cgaggggtcc tcacctggtt gacccaaaaa tgttcccttg accattggct gcgctaaccc 1140
ccagcccaca gagcctgaat ttgtaagcaa cttgcttcta aatgcccagt tcacttcttt 1200
gcagagcctt ttacccctgc acagtttaga acagagggac caaattgctt ctaggagtca 1260
actggctggc cagtctgggt ctgggtttgg atctccaatt gcctcttgca ggctgagtcc 1320
ctccatgcaa aagtggggct aaatgaagtg tgttaagggg tcggctaagt gggacattag 1380
taactgcaca ctatttccct ctactgagta aaccctatct gtgattcccc caaacatctg 1440
gcatggctcc cttttgtcct tcctgtgccc tgcaaatatt agcaaagaag cttcatgcca 1500
ggttaggaag gcagcattcc atgaccagaa acagggacaa agaaatcccc ccttcagaac 1560
agaggcattt aaaatggaaa agagagattg gattttggtg ggtaacttag aaggatggca 1620
tctccatgta gaataaatga agaaagggag gcccagccgc aggaaggcag aataaatcct 1680
tgggagtcat taccacgcct tgaccttccc aaggttactc agcagcagag agccctgggt 1740
gacttcaggt ggagagcact agaagtggtt tcctgataac aagcaaggat atcagagctg 1800
ggaaattcat gtggatctgg ggactgagtg tgggagtgca gagaaagaaa gggaaactgg 1860
ctgaggggat accataaaaa gaggatgatt tcagaaggag aaggaaaaag aaagtaatgc 1920
cacacattgt gcttggcccc tggtaagcag aggctttggg gtcctagccc agtgcttctc 1980
caacactgaa gtgcttgcag atcatctggg gacctggttt gaatggagat tctgattcag 2040
tgggttgggg gcagagtttc tgcagttcca tcaggtcccc cccaggtgca ggtgctgaca 2100
atactgctgc cttacccgcc atacattaag gagcagggtc ctggtcctaa agagttattc 2160
aaatgaaggt ggttcgacgc cccgaacctc acctgacctc aactaaccct taaaaatgca 2220
cacctcatga gtctacctga gcattcaggc agcactgaca atagttatgc ctgtactaag 2280
gagcatgatt ttaagaggct ttggcccaat gcctataaaa tgcccatttc gaagatatac 2340
aaaaacatac ttcaaaaatg ttaaaccctt accaacagct tttcccagga gaccatttgt 2400
attaccatta cttgtataaa tacacttcct gcttaaactt gacccaggtg gctagcaaat 2460
tagaaacacc attcatctct aacatatgat actgatgcca tgtaaaggcc tttaataagt 2520
cattgaaatt tactgtgaga ctgtatgttt taattgcatt taaaaatata tagcttgaaa 2580
gcagttaaac tgattagtat tcaggcactg agaatgatag taataggata caatgtataa 2640
gctactcact tatctgatac ttatttacct ataaaatgag atttttgttt tccactgtgc 2700
tattacaaat tttcttttga aagtaggaac tcttaagcaa tggtaattgt gaataaaaat 2760
tgatgagagt gttagctcct gtttcatatg aaattgaagt aattgttaac taaaaacaat 2820
tccttagtaa ctgaactgtc atatttagaa tggaaggaaa atgacagttt gtgaaagttc 2880
aaagcaatag tgcaattgaa gaattgacct aagtaagctg acattatggt taataatagt 2940
attttagatt tgtgcagcaa aataatttca taactttttt gtttttgtta cttggataag 3000
atcaatctgt tttattttag taaatctttg caggcaagtt agagaaaatg cagtgtggct 3060
taacgtctct ttagtatgaa gatttggcca gaaaaagata cccagagagg aaatctaaga 3120
taattataat ggtccatact ttttattgta tgaatcaaac tcaagcataa cattggccaa 3180
ggaaaattaa ataccattgc taacttgtga aatggaagtc tgtgatttcg gagatgcaaa 3240
gcattgtagt aaaaacacca atgtgacctc gaccatctca gcccagatat cattcatata 3300
tctgttcaat gactattaag gtgcctactg tgtgctaggc actgtactgg atactgggga 3360
ccttgtctgt ctggtttgct gctgtatctt ctcccagggc attatattta tgatgaaaga 3420
tgctgtggat tcaattcttt cagtcaagaa taaacacaga ctttgtaggt tcctgctgaa 3480
taaagcaaat cccagaaacc cagattttgg aagaatcagc aaccccagca taaaataaac 3540
ccctatcaaa atgtcagagg acatggcaag gtaaacttag cattttcaac tttagaaccg 3600
ggtcagcttc agggggactg ctttcaaatc agccaaagag cctgtcagat cttcttagaa 3660
ggaagaggtt ggtagttccc tgctctgttt tgaacatgct ctagtttatt aacctgggga 3720
cattcccatt gctgtcttaa gtaagtctca tagccagctc ctgtcacgtg actctcatat 3780
ggattcattt tcgggccagc tctgaacaaa gcatcatgaa catatgtgct tttggtcgtt 3840
tgcaatgtga tggtggtgga ggtaggtatt ggtttccttg gaaggcatga taagaaagat 3900
tcacaatggc caacagtgtg tatgaacaaa aaactgattg gagcatcagc tagtactgaa 3960
ggtccttgct ttgtgtcaga ggcaaaggaa cccaaggcgc caagtcctca gccttgagtg 4020
tactgctgac aactaaactc acaggctgca aagcagacct ctgatgaaga tgcctgttat 4080
ttcacatcac tgtctttttg tgtatcatag tctgcacctt acaaatatta ataaatgttc 4140
caataatagg tgaaaaaaaa aa 4162
<210> SEQ ID NO 41
<211> LENGTH: 4058
<212> TYPE: DNA
<213> ORGANISM: Homo sapiens
<400> SEQUENCE: 41
agaagtccat tcggctcaca catttgcccc aagacaaacc acgttaaaat aacacccagg 60
gtagctgctg ccaccgtctt ctgtctctac ctccctcctg gctggccaat ggctctgtgt 120
tcctgggcct gctgctggct gtccagagta ggggttgctt agagctgtgt gcatccctgc 180
gggtggtgtg ggagtgggcg gttgtctaaa ggcaggtccc ctctactgat aaacaaggac 240
cggagataga cctagaggct gacattcttg gctcccccag cctacacccc ccccacctcg 300
atttcccaca gagccctagg gacgggtagc cagctctgtg gcatggtatc tggaggcagg 360
ccagcaacct gatgtgcatg ccacggcccg tccctctccc cactcagagc tgcagtagcc 420
tggaggttca gagagccggg ctactctgag aagaagacac caagtggatt ctgcttcccc 480
tgggacagca ctgagcgagt gtggagagag gtacagccct cggcctacaa gctctttagt 540
cttgaaagcg ccacaagcag cagctgctga gccatggctg aaggggaaat caccaccttc 600
acagccctga ccgagaagtt taatctgcct ccagggaatt acaagaagcc caaactcctc 660
tactgtagca acgggggcca cttcctgagg atccttccgg atggcacagt ggatgggaca 720
agggacagga gcgaccagca cacagacacc aaatgaggaa tgtttgttcc tggaaaggct 780
ggaggagaac cattacaaca cctatatatc caagaagcat gcagagaaga attggtttgt 840
tggcctcaag aagaatggga gctgcaaacg cggtcctcgg actcactatg gccagaaagc 900
aatcttgttt ctccccctgc cagtctcttc tgattaaaga gatctgttct gggtgttgac 960
cactccagag aagtttcgag gggtcctcac ctggttgacc caaaaatgtt cccttgacca 1020
ttggctgcgc taacccccag cccacagagc ctgaatttgt aagcaacttg cttctaaatg 1080
cccagttcac ttctttgcag agccttttac ccctgcacag tttagaacag agggaccaaa 1140
ttgcttctag gagtcaactg gctggccagt ctgggtctgg gtttggatct ccaattgcct 1200
cttgcaggct gagtccctcc atgcaaaagt ggggctaaat gaagtgtgtt aaggggtcgg 1260
ctaagtggga cattagtaac tgcacactat ttccctctac tgagtaaacc ctatctgtga 1320
ttcccccaaa catctggcat ggctcccttt tgtccttcct gtgccctgca aatattagca 1380
aagaagcttc atgccaggtt aggaaggcag cattccatga ccagaaacag ggacaaagaa 1440
atcccccctt cagaacagag gcatttaaaa tggaaaagag agattggatt ttggtgggta 1500
acttagaagg atggcatctc catgtagaat aaatgaagaa agggaggccc agccgcagga 1560
aggcagaata aatccttggg agtcattacc acgccttgac cttcccaagg ttactcagca 1620
gcagagagcc ctgggtgact tcaggtggag agcactagaa gtggtttcct gataacaagc 1680
aaggatatca gagctgggaa attcatgtgg atctggggac tgagtgtggg agtgcagaga 1740
aagaaaggga aactggctga ggggatacca taaaaagagg atgatttcag aaggagaagg 1800
aaaaagaaag taatgccaca cattgtgctt ggcccctggt aagcagaggc tttggggtcc 1860
tagcccagtg cttctccaac actgaagtgc ttgcagatca tctggggacc tggtttgaat 1920
ggagattctg attcagtggg ttgggggcag agtttctgca gttccatcag gtccccccca 1980
ggtgcaggtg ctgacaatac tgctgcctta cccgccatac attaaggagc agggtcctgg 2040
tcctaaagag ttattcaaat gaaggtggtt cgacgccccg aacctcacct gacctcaact 2100
aacccttaaa aatgcacacc tcatgagtct acctgagcat tcaggcagca ctgacaatag 2160
ttatgcctgt actaaggagc atgattttaa gaggctttgg cccaatgcct ataaaatgcc 2220
catttcgaag atatacaaaa acatacttca aaaatgttaa acccttacca acagcttttc 2280
ccaggagacc atttgtatta ccattacttg tataaataca cttcctgctt aaacttgacc 2340
caggtggcta gcaaattaga aacaccattc atctctaaca tatgatactg atgccatgta 2400
aaggccttta ataagtcatt gaaatttact gtgagactgt atgttttaat tgcatttaaa 2460
aatatatagc ttgaaagcag ttaaactgat tagtattcag gcactgagaa tgatagtaat 2520
aggatacaat gtataagcta ctcacttatc tgatacttat ttacctataa aatgagattt 2580
ttgttttcca ctgtgctatt acaaattttc ttttgaaagt aggaactctt aagcaatggt 2640
aattgtgaat aaaaattgat gagagtgtta gctcctgttt catatgaaat tgaagtaatt 2700
gttaactaaa aacaattcct tagtaactga actgtcatat ttagaatgga aggaaaatga 2760
cagtttgtga aagttcaaag caatagtgca attgaagaat tgacctaagt aagctgacat 2820
tatggttaat aatagtattt tagatttgtg cagcaaaata atttcataac ttttttgttt 2880
ttgttacttg gataagatca atctgtttta ttttagtaaa tctttgcagg caagttagag 2940
aaaatgcagt gtggcttaac gtctctttag tatgaagatt tggccagaaa aagataccca 3000
gagaggaaat ctaagataat tataatggtc catacttttt attgtatgaa tcaaactcaa 3060
gcataacatt ggccaaggaa aattaaatac cattgctaac ttgtgaaatg gaagtctgtg 3120
atttcggaga tgcaaagcat tgtagtaaaa acaccaatgt gacctcgacc atctcagccc 3180
agatatcatt catatatctg ttcaatgact attaaggtgc ctactgtgtg ctaggcactg 3240
tactggatac tggggacctt gtctgtctgg tttgctgctg tatcttctcc cagggcatta 3300
tatttatgat gaaagatgct gtggattcaa ttctttcagt caagaataaa cacagacttt 3360
gtaggttcct gctgaataaa gcaaatccca gaaacccaga ttttggaaga atcagcaacc 3420
ccagcataaa ataaacccct atcaaaatgt cagaggacat ggcaaggtaa acttagcatt 3480
ttcaacttta gaaccgggtc agcttcaggg ggactgcttt caaatcagcc aaagagcctg 3540
tcagatcttc ttagaaggaa gaggttggta gttccctgct ctgttttgaa catgctctag 3600
tttattaacc tggggacatt cccattgctg tcttaagtaa gtctcatagc cagctcctgt 3660
cacgtgactc tcatatggat tcattttcgg gccagctctg aacaaagcat catgaacata 3720
tgtgcttttg gtcgtttgca atgtgatggt ggtggaggta ggtattggtt tccttggaag 3780
gcatgataag aaagattcac aatggccaac agtgtgtatg aacaaaaaac tgattggagc 3840
atcagctagt actgaaggtc cttgctttgt gtcagaggca aaggaaccca aggcgccaag 3900
tcctcagcct tgagtgtact gctgacaact aaactcacag gctgcaaagc agacctctga 3960
tgaagatgcc tgttatttca catcactgtc tttttgtgta tcatagtctg caccttacaa 4020
atattaataa atgttccaat aataggtgaa aaaaaaaa 4058
<210> SEQ ID NO 42
<211> LENGTH: 3516
<212> TYPE: DNA
<213> ORGANISM: Homo sapiens
<400> SEQUENCE: 42
tcttgaaagc gccacaagca gcagctgctg agccatggct gaaggggaaa tcaccacctt 60
cacagccctg accgagaagt ttaatctgcc tccagggaat tacaagaagc ccaaactcct 120
ctactgtagc aacgggggcc acttcctgag gatccttccg gatggcacag tggatgggac 180
aagggacagg agcgaccagc acaacaccaa atgaggaatg tttgttcctg gaaaggctgg 240
aggagaacca ttacaacacc tatatatcca agaagcatgc agagaagaat tggtttgttg 300
gcctcaagaa gaatgggagc tgcaaacgcg gtcctcggac tcactatggc cagaaagcaa 360
tcttgtttct ccccctgcca gtctcttctg attaaagaga tctgttctgg gtgttgacca 420
ctccagagaa gtttcgaggg gtcctcacct ggttgaccca aaaatgttcc cttgaccatt 480
ggctgcgcta acccccagcc cacagagcct gaatttgtaa gcaacttgct tctaaatgcc 540
cagttcactt ctttgcagag ccttttaccc ctgcacagtt tagaacagag ggaccaaatt 600
gcttctagga gtcaactggc tggccagtct gggtctgggt ttggatctcc aattgcctct 660
tgcaggctga gtccctccat gcaaaagtgg ggctaaatga agtgtgttaa ggggtcggct 720
aagtgggaca ttagtaactg cacactattt ccctctactg agtaaaccct atctgtgatt 780
cccccaaaca tctggcatgg ctcccttttg tccttcctgt gccctgcaaa tattagcaaa 840
gaagcttcat gccaggttag gaaggcagca ttccatgacc agaaacaggg acaaagaaat 900
ccccccttca gaacagaggc atttaaaatg gaaaagagag attggatttt ggtgggtaac 960
ttagaaggat ggcatctcca tgtagaataa atgaagaaag ggaggcccag ccgcaggaag 1020
gcagaataaa tccttgggag tcattaccac gccttgacct tcccaaggtt actcagcagc 1080
agagagccct gggtgacttc aggtggagag cactagaagt ggtttcctga taacaagcaa 1140
ggatatcaga gctgggaaat tcatgtggat ctggggactg agtgtgggag tgcagagaaa 1200
gaaagggaaa ctggctgagg ggataccata aaaagaggat gatttcagaa ggagaaggaa 1260
aaagaaagta atgccacaca ttgtgcttgg cccctggtaa gcagaggctt tggggtccta 1320
gcccagtgct tctccaacac tgaagtgctt gcagatcatc tggggacctg gtttgaatgg 1380
agattctgat tcagtgggtt gggggcagag tttctgcagt tccatcaggt cccccccagg 1440
tgcaggtgct gacaatactg ctgccttacc cgccatacat taaggagcag ggtcctggtc 1500
ctaaagagtt attcaaatga aggtggttcg acgccccgaa cctcacctga cctcaactaa 1560
cccttaaaaa tgcacacctc atgagtctac ctgagcattc aggcagcact gacaatagtt 1620
atgcctgtac taaggagcat gattttaaga ggctttggcc caatgcctat aaaatgccca 1680
tttcgaagat atacaaaaac atacttcaaa aatgttaaac ccttaccaac agcttttccc 1740
aggagaccat ttgtattacc attacttgta taaatacact tcctgcttaa acttgaccca 1800
ggtggctagc aaattagaaa caccattcat ctctaacata tgatactgat gccatgtaaa 1860
ggcctttaat aagtcattga aatttactgt gagactgtat gttttaattg catttaaaaa 1920
tatatagctt gaaagcagtt aaactgatta gtattcaggc actgagaatg atagtaatag 1980
gatacaatgt ataagctact cacttatctg atacttattt acctataaaa tgagattttt 2040
gttttccact gtgctattac aaattttctt ttgaaagtag gaactcttaa gcaatggtaa 2100
ttgtgaataa aaattgatga gagtgttagc tcctgtttca tatgaaattg aagtaattgt 2160
taactaaaaa caattcctta gtaactgaac tgtcatattt agaatggaag gaaaatgaca 2220
gtttgtgaaa gttcaaagca atagtgcaat tgaagaattg acctaagtaa gctgacatta 2280
tggttaataa tagtatttta gatttgtgca gcaaaataat ttcataactt ttttgttttt 2340
gttacttgga taagatcaat ctgttttatt ttagtaaatc tttgcaggca agttagagaa 2400
aatgcagtgt ggcttaacgt ctctttagta tgaagatttg gccagaaaaa gatacccaga 2460
gaggaaatct aagataatta taatggtcca tactttttat tgtatgaatc aaactcaagc 2520
ataacattgg ccaaggaaaa ttaaatacca ttgctaactt gtgaaatgga agtctgtgat 2580
ttcggagatg caaagcattg tagtaaaaac accaatgtga cctcgaccat ctcagcccag 2640
atatcattca tatatctgtt caatgactat taaggtgcct actgtgtgct aggcactgta 2700
ctggatactg gggaccttgt ctgtctggtt tgctgctgta tcttctccca gggcattata 2760
tttatgatga aagatgctgt ggattcaatt ctttcagtca agaataaaca cagactttgt 2820
aggttcctgc tgaataaagc aaatcccaga aacccagatt ttggaagaat cagcaacccc 2880
agcataaaat aaacccctat caaaatgtca gaggacatgg caaggtaaac ttagcatttt 2940
caactttaga accgggtcag cttcaggggg actgctttca aatcagccaa agagcctgtc 3000
agatcttctt agaaggaaga ggttggtagt tccctgctct gttttgaaca tgctctagtt 3060
tattaacctg gggacattcc cattgctgtc ttaagtaagt ctcatagcca gctcctgtca 3120
cgtgactctc atatggattc attttcgggc cagctctgaa caaagcatca tgaacatatg 3180
tgcttttggt cgtttgcaat gtgatggtgg tggaggtagg tattggtttc cttggaaggc 3240
atgataagaa agattcacaa tggccaacag tgtgtatgaa caaaaaactg attggagcat 3300
cagctagtac tgaaggtcct tgctttgtgt cagaggcaaa ggaacccaag gcgccaagtc 3360
ctcagccttg agtgtactgc tgacaactaa actcacaggc tgcaaagcag acctctgatg 3420
aagatgcctg ttatttcaca tcactgtctt tttgtgtatc atagtctgca ccttacaaat 3480
attaataaat gttccaataa taggtgaaaa aaaaaa 3516
<210> SEQ ID NO 43
<211> LENGTH: 3682
<212> TYPE: DNA
<213> ORGANISM: Homo sapiens
<400> SEQUENCE: 43
aaaaagagag agagaaaaaa tactgttggc agcagcacaa tgtttgggct aagacctggt 60
cttgaaagcg ccacaagcag cagctgctga gccatggctg aaggggaaat caccaccttc 120
acagccctga ccgagaagtt taatctgcct ccagggaatt acaagaagcc caaactcctc 180
tactgtagca acgggggcca cttcctgagg atccttccgg atggcacagt ggatgggaca 240
agggacagga gcgaccagca cattcagctg cagctcagtg cggaaagcgt gggggaggtg 300
tatataaaga gtaccgagac tggccagtac ttggccatgg acaccgacgg gcttttatac 360
ggctcacaga caccaaatga ggaatgtttg ttcctggaaa ggctggagga gaaccattac 420
aacacctata tatccaagaa gcatgcagag aagaattggt ttgttggcct caagaagaat 480
gggagctgca aacgcggtcc tcggactcac tatggccaga aagcaatctt gtttctcccc 540
ctgccagtct cttctgatta aagagatctg ttctgggtgt tgaccactcc agagaagttt 600
cgaggggtcc tcacctggtt gacccaaaaa tgttcccttg accattggct gcgctaaccc 660
ccagcccaca gagcctgaat ttgtaagcaa cttgcttcta aatgcccagt tcacttcttt 720
gcagagcctt ttacccctgc acagtttaga acagagggac caaattgctt ctaggagtca 780
actggctggc cagtctgggt ctgggtttgg atctccaatt gcctcttgca ggctgagtcc 840
ctccatgcaa aagtggggct aaatgaagtg tgttaagggg tcggctaagt gggacattag 900
taactgcaca ctatttccct ctactgagta aaccctatct gtgattcccc caaacatctg 960
gcatggctcc cttttgtcct tcctgtgccc tgcaaatatt agcaaagaag cttcatgcca 1020
ggttaggaag gcagcattcc atgaccagaa acagggacaa agaaatcccc ccttcagaac 1080
agaggcattt aaaatggaaa agagagattg gattttggtg ggtaacttag aaggatggca 1140
tctccatgta gaataaatga agaaagggag gcccagccgc aggaaggcag aataaatcct 1200
tgggagtcat taccacgcct tgaccttccc aaggttactc agcagcagag agccctgggt 1260
gacttcaggt ggagagcact agaagtggtt tcctgataac aagcaaggat atcagagctg 1320
ggaaattcat gtggatctgg ggactgagtg tgggagtgca gagaaagaaa gggaaactgg 1380
ctgaggggat accataaaaa gaggatgatt tcagaaggag aaggaaaaag aaagtaatgc 1440
cacacattgt gcttggcccc tggtaagcag aggctttggg gtcctagccc agtgcttctc 1500
caacactgaa gtgcttgcag atcatctggg gacctggttt gaatggagat tctgattcag 1560
tgggttgggg gcagagtttc tgcagttcca tcaggtcccc cccaggtgca ggtgctgaca 1620
atactgctgc cttacccgcc atacattaag gagcagggtc ctggtcctaa agagttattc 1680
aaatgaaggt ggttcgacgc cccgaacctc acctgacctc aactaaccct taaaaatgca 1740
cacctcatga gtctacctga gcattcaggc agcactgaca atagttatgc ctgtactaag 1800
gagcatgatt ttaagaggct ttggcccaat gcctataaaa tgcccatttc gaagatatac 1860
aaaaacatac ttcaaaaatg ttaaaccctt accaacagct tttcccagga gaccatttgt 1920
attaccatta cttgtataaa tacacttcct gcttaaactt gacccaggtg gctagcaaat 1980
tagaaacacc attcatctct aacatatgat actgatgcca tgtaaaggcc tttaataagt 2040
cattgaaatt tactgtgaga ctgtatgttt taattgcatt taaaaatata tagcttgaaa 2100
gcagttaaac tgattagtat tcaggcactg agaatgatag taataggata caatgtataa 2160
gctactcact tatctgatac ttatttacct ataaaatgag atttttgttt tccactgtgc 2220
tattacaaat tttcttttga aagtaggaac tcttaagcaa tggtaattgt gaataaaaat 2280
tgatgagagt gttagctcct gtttcatatg aaattgaagt aattgttaac taaaaacaat 2340
tccttagtaa ctgaactgtc atatttagaa tggaaggaaa atgacagttt gtgaaagttc 2400
aaagcaatag tgcaattgaa gaattgacct aagtaagctg acattatggt taataatagt 2460
attttagatt tgtgcagcaa aataatttca taactttttt gtttttgtta cttggataag 2520
atcaatctgt tttattttag taaatctttg caggcaagtt agagaaaatg cagtgtggct 2580
taacgtctct ttagtatgaa gatttggcca gaaaaagata cccagagagg aaatctaaga 2640
taattataat ggtccatact ttttattgta tgaatcaaac tcaagcataa cattggccaa 2700
ggaaaattaa ataccattgc taacttgtga aatggaagtc tgtgatttcg gagatgcaaa 2760
gcattgtagt aaaaacacca atgtgacctc gaccatctca gcccagatat cattcatata 2820
tctgttcaat gactattaag gtgcctactg tgtgctaggc actgtactgg atactgggga 2880
ccttgtctgt ctggtttgct gctgtatctt ctcccagggc attatattta tgatgaaaga 2940
tgctgtggat tcaattcttt cagtcaagaa taaacacaga ctttgtaggt tcctgctgaa 3000
taaagcaaat cccagaaacc cagattttgg aagaatcagc aaccccagca taaaataaac 3060
ccctatcaaa atgtcagagg acatggcaag gtaaacttag cattttcaac tttagaaccg 3120
ggtcagcttc agggggactg ctttcaaatc agccaaagag cctgtcagat cttcttagaa 3180
ggaagaggtt ggtagttccc tgctctgttt tgaacatgct ctagtttatt aacctgggga 3240
cattcccatt gctgtcttaa gtaagtctca tagccagctc ctgtcacgtg actctcatat 3300
ggattcattt tcgggccagc tctgaacaaa gcatcatgaa catatgtgct tttggtcgtt 3360
tgcaatgtga tggtggtgga ggtaggtatt ggtttccttg gaaggcatga taagaaagat 3420
tcacaatggc caacagtgtg tatgaacaaa aaactgattg gagcatcagc tagtactgaa 3480
ggtccttgct ttgtgtcaga ggcaaaggaa cccaaggcgc caagtcctca gccttgagtg 3540
tactgctgac aactaaactc acaggctgca aagcagacct ctgatgaaga tgcctgttat 3600
ttcacatcac tgtctttttg tgtatcatag tctgcacctt acaaatatta ataaatgttc 3660
caataatagg tgaaaaaaaa aa 3682
<210> SEQ ID NO 44
<211> LENGTH: 3875
<212> TYPE: DNA
<213> ORGANISM: Homo sapiens
<400> SEQUENCE: 44
acatgagagg gggagaaata aatatacagt gcttgtcctt agcctttctg tgggcatacc 60
agtgtcagct gcacttgtag gggcccaagt gcctcatgac ccactcggca gccttcctct 120
ccaggatccc caaggctagg aggccaacct actaacagca gcctgcctgc agctgtcctg 180
gtagaacagt gtggacattg cagaagctgt cactgcccca gaaagaaagc accccagagc 240
caaggcaaag agtcttgaaa gcgccacaag cagcagctgc tgagccatgg ctgaagggga 300
aatcaccacc ttcacagccc tgaccgagaa gtttaatctg cctccaggga attacaagaa 360
gcccaaactc ctctactgta gcaacggggg ccacttcctg aggatccttc cggatggcac 420
agtggatggg acaagggaca ggagcgacca gcacattcag ctgcagctca gtgcggaaag 480
cgtgggggag gtgtatataa agagtaccga gactggccag tacttggcca tggacaccga 540
cgggctttta tacggctcac agacaccaaa tgaggaatgt ttgttcctgg aaaggctgga 600
ggagaaccat tacaacacct atatatccaa gaagcatgca gagaagaatt ggtttgttgg 660
cctcaagaag aatgggagct gcaaacgcgg tcctcggact cactatggcc agaaagcaat 720
cttgtttctc cccctgccag tctcttctga ttaaagagat ctgttctggg tgttgaccac 780
tccagagaag tttcgagggg tcctcacctg gttgacccaa aaatgttccc ttgaccattg 840
gctgcgctaa cccccagccc acagagcctg aatttgtaag caacttgctt ctaaatgccc 900
agttcacttc tttgcagagc cttttacccc tgcacagttt agaacagagg gaccaaattg 960
cttctaggag tcaactggct ggccagtctg ggtctgggtt tggatctcca attgcctctt 1020
gcaggctgag tccctccatg caaaagtggg gctaaatgaa gtgtgttaag gggtcggcta 1080
agtgggacat tagtaactgc acactatttc cctctactga gtaaacccta tctgtgattc 1140
ccccaaacat ctggcatggc tcccttttgt ccttcctgtg ccctgcaaat attagcaaag 1200
aagcttcatg ccaggttagg aaggcagcat tccatgacca gaaacaggga caaagaaatc 1260
cccccttcag aacagaggca tttaaaatgg aaaagagaga ttggattttg gtgggtaact 1320
tagaaggatg gcatctccat gtagaataaa tgaagaaagg gaggcccagc cgcaggaagg 1380
cagaataaat ccttgggagt cattaccacg ccttgacctt cccaaggtta ctcagcagca 1440
gagagccctg ggtgacttca ggtggagagc actagaagtg gtttcctgat aacaagcaag 1500
gatatcagag ctgggaaatt catgtggatc tggggactga gtgtgggagt gcagagaaag 1560
aaagggaaac tggctgaggg gataccataa aaagaggatg atttcagaag gagaaggaaa 1620
aagaaagtaa tgccacacat tgtgcttggc ccctggtaag cagaggcttt ggggtcctag 1680
cccagtgctt ctccaacact gaagtgcttg cagatcatct ggggacctgg tttgaatgga 1740
gattctgatt cagtgggttg ggggcagagt ttctgcagtt ccatcaggtc ccccccaggt 1800
gcaggtgctg acaatactgc tgccttaccc gccatacatt aaggagcagg gtcctggtcc 1860
taaagagtta ttcaaatgaa ggtggttcga cgccccgaac ctcacctgac ctcaactaac 1920
ccttaaaaat gcacacctca tgagtctacc tgagcattca ggcagcactg acaatagtta 1980
tgcctgtact aaggagcatg attttaagag gctttggccc aatgcctata aaatgcccat 2040
ttcgaagata tacaaaaaca tacttcaaaa atgttaaacc cttaccaaca gcttttccca 2100
ggagaccatt tgtattacca ttacttgtat aaatacactt cctgcttaaa cttgacccag 2160
gtggctagca aattagaaac accattcatc tctaacatat gatactgatg ccatgtaaag 2220
gcctttaata agtcattgaa atttactgtg agactgtatg ttttaattgc atttaaaaat 2280
atatagcttg aaagcagtta aactgattag tattcaggca ctgagaatga tagtaatagg 2340
atacaatgta taagctactc acttatctga tacttattta cctataaaat gagatttttg 2400
ttttccactg tgctattaca aattttcttt tgaaagtagg aactcttaag caatggtaat 2460
tgtgaataaa aattgatgag agtgttagct cctgtttcat atgaaattga agtaattgtt 2520
aactaaaaac aattccttag taactgaact gtcatattta gaatggaagg aaaatgacag 2580
tttgtgaaag ttcaaagcaa tagtgcaatt gaagaattga cctaagtaag ctgacattat 2640
ggttaataat agtattttag atttgtgcag caaaataatt tcataacttt tttgtttttg 2700
ttacttggat aagatcaatc tgttttattt tagtaaatct ttgcaggcaa gttagagaaa 2760
atgcagtgtg gcttaacgtc tctttagtat gaagatttgg ccagaaaaag atacccagag 2820
aggaaatcta agataattat aatggtccat actttttatt gtatgaatca aactcaagca 2880
taacattggc caaggaaaat taaataccat tgctaacttg tgaaatggaa gtctgtgatt 2940
tcggagatgc aaagcattgt agtaaaaaca ccaatgtgac ctcgaccatc tcagcccaga 3000
tatcattcat atatctgttc aatgactatt aaggtgccta ctgtgtgcta ggcactgtac 3060
tggatactgg ggaccttgtc tgtctggttt gctgctgtat cttctcccag ggcattatat 3120
ttatgatgaa agatgctgtg gattcaattc tttcagtcaa gaataaacac agactttgta 3180
ggttcctgct gaataaagca aatcccagaa acccagattt tggaagaatc agcaacccca 3240
gcataaaata aacccctatc aaaatgtcag aggacatggc aaggtaaact tagcattttc 3300
aactttagaa ccgggtcagc ttcaggggga ctgctttcaa atcagccaaa gagcctgtca 3360
gatcttctta gaaggaagag gttggtagtt ccctgctctg ttttgaacat gctctagttt 3420
attaacctgg ggacattccc attgctgtct taagtaagtc tcatagccag ctcctgtcac 3480
gtgactctca tatggattca ttttcgggcc agctctgaac aaagcatcat gaacatatgt 3540
gcttttggtc gtttgcaatg tgatggtggt ggaggtaggt attggtttcc ttggaaggca 3600
tgataagaaa gattcacaat ggccaacagt gtgtatgaac aaaaaactga ttggagcatc 3660
agctagtact gaaggtcctt gctttgtgtc agaggcaaag gaacccaagg cgccaagtcc 3720
tcagccttga gtgtactgct gacaactaaa ctcacaggct gcaaagcaga cctctgatga 3780
agatgcctgt tatttcacat cactgtcttt ttgtgtatca tagtctgcac cttacaaata 3840
ttaataaatg ttccaataat aggtgaaaaa aaaaa 3875
<210> SEQ ID NO 45
<211> LENGTH: 3781
<212> TYPE: DNA
<213> ORGANISM: Homo sapiens
<400> SEQUENCE: 45
acatgagagg gggagaaata aatatacagt gcttgtcctt agcctttctg tgggcatacc 60
agtgtcagct gcacttgtag gggcccaagt gcctcatgac ccactcggca gccttcctct 120
ccaggatccc caaggctagg aggccaacct actaacagtc ttgaaagcgc cacaagcagc 180
agctgctgag ccatggctga aggggaaatc accaccttca cagccctgac cgagaagttt 240
aatctgcctc cagggaatta caagaagccc aaactcctct actgtagcaa cgggggccac 300
ttcctgagga tccttccgga tggcacagtg gatgggacaa gggacaggag cgaccagcac 360
attcagctgc agctcagtgc ggaaagcgtg ggggaggtgt atataaagag taccgagact 420
ggccagtact tggccatgga caccgacggg cttttatacg gctcacagac accaaatgag 480
gaatgtttgt tcctggaaag gctggaggag aaccattaca acacctatat atccaagaag 540
catgcagaga agaattggtt tgttggcctc aagaagaatg ggagctgcaa acgcggtcct 600
cggactcact atggccagaa agcaatcttg tttctccccc tgccagtctc ttctgattaa 660
agagatctgt tctgggtgtt gaccactcca gagaagtttc gaggggtcct cacctggttg 720
acccaaaaat gttcccttga ccattggctg cgctaacccc cagcccacag agcctgaatt 780
tgtaagcaac ttgcttctaa atgcccagtt cacttctttg cagagccttt tacccctgca 840
cagtttagaa cagagggacc aaattgcttc taggagtcaa ctggctggcc agtctgggtc 900
tgggtttgga tctccaattg cctcttgcag gctgagtccc tccatgcaaa agtggggcta 960
aatgaagtgt gttaaggggt cggctaagtg ggacattagt aactgcacac tatttccctc 1020
tactgagtaa accctatctg tgattccccc aaacatctgg catggctccc ttttgtcctt 1080
cctgtgccct gcaaatatta gcaaagaagc ttcatgccag gttaggaagg cagcattcca 1140
tgaccagaaa cagggacaaa gaaatccccc cttcagaaca gaggcattta aaatggaaaa 1200
gagagattgg attttggtgg gtaacttaga aggatggcat ctccatgtag aataaatgaa 1260
gaaagggagg cccagccgca ggaaggcaga ataaatcctt gggagtcatt accacgcctt 1320
gaccttccca aggttactca gcagcagaga gccctgggtg acttcaggtg gagagcacta 1380
gaagtggttt cctgataaca agcaaggata tcagagctgg gaaattcatg tggatctggg 1440
gactgagtgt gggagtgcag agaaagaaag ggaaactggc tgaggggata ccataaaaag 1500
aggatgattt cagaaggaga aggaaaaaga aagtaatgcc acacattgtg cttggcccct 1560
ggtaagcaga ggctttgggg tcctagccca gtgcttctcc aacactgaag tgcttgcaga 1620
tcatctgggg acctggtttg aatggagatt ctgattcagt gggttggggg cagagtttct 1680
gcagttccat caggtccccc ccaggtgcag gtgctgacaa tactgctgcc ttacccgcca 1740
tacattaagg agcagggtcc tggtcctaaa gagttattca aatgaaggtg gttcgacgcc 1800
ccgaacctca cctgacctca actaaccctt aaaaatgcac acctcatgag tctacctgag 1860
cattcaggca gcactgacaa tagttatgcc tgtactaagg agcatgattt taagaggctt 1920
tggcccaatg cctataaaat gcccatttcg aagatataca aaaacatact tcaaaaatgt 1980
taaaccctta ccaacagctt ttcccaggag accatttgta ttaccattac ttgtataaat 2040
acacttcctg cttaaacttg acccaggtgg ctagcaaatt agaaacacca ttcatctcta 2100
acatatgata ctgatgccat gtaaaggcct ttaataagtc attgaaattt actgtgagac 2160
tgtatgtttt aattgcattt aaaaatatat agcttgaaag cagttaaact gattagtatt 2220
caggcactga gaatgatagt aataggatac aatgtataag ctactcactt atctgatact 2280
tatttaccta taaaatgaga tttttgtttt ccactgtgct attacaaatt ttcttttgaa 2340
agtaggaact cttaagcaat ggtaattgtg aataaaaatt gatgagagtg ttagctcctg 2400
tttcatatga aattgaagta attgttaact aaaaacaatt ccttagtaac tgaactgtca 2460
tatttagaat ggaaggaaaa tgacagtttg tgaaagttca aagcaatagt gcaattgaag 2520
aattgaccta agtaagctga cattatggtt aataatagta ttttagattt gtgcagcaaa 2580
ataatttcat aacttttttg tttttgttac ttggataaga tcaatctgtt ttattttagt 2640
aaatctttgc aggcaagtta gagaaaatgc agtgtggctt aacgtctctt tagtatgaag 2700
atttggccag aaaaagatac ccagagagga aatctaagat aattataatg gtccatactt 2760
tttattgtat gaatcaaact caagcataac attggccaag gaaaattaaa taccattgct 2820
aacttgtgaa atggaagtct gtgatttcgg agatgcaaag cattgtagta aaaacaccaa 2880
tgtgacctcg accatctcag cccagatatc attcatatat ctgttcaatg actattaagg 2940
tgcctactgt gtgctaggca ctgtactgga tactggggac cttgtctgtc tggtttgctg 3000
ctgtatcttc tcccagggca ttatatttat gatgaaagat gctgtggatt caattctttc 3060
agtcaagaat aaacacagac tttgtaggtt cctgctgaat aaagcaaatc ccagaaaccc 3120
agattttgga agaatcagca accccagcat aaaataaacc cctatcaaaa tgtcagagga 3180
catggcaagg taaacttagc attttcaact ttagaaccgg gtcagcttca gggggactgc 3240
tttcaaatca gccaaagagc ctgtcagatc ttcttagaag gaagaggttg gtagttccct 3300
gctctgtttt gaacatgctc tagtttatta acctggggac attcccattg ctgtcttaag 3360
taagtctcat agccagctcc tgtcacgtga ctctcatatg gattcatttt cgggccagct 3420
ctgaacaaag catcatgaac atatgtgctt ttggtcgttt gcaatgtgat ggtggtggag 3480
gtaggtattg gtttccttgg aaggcatgat aagaaagatt cacaatggcc aacagtgtgt 3540
atgaacaaaa aactgattgg agcatcagct agtactgaag gtccttgctt tgtgtcagag 3600
gcaaaggaac ccaaggcgcc aagtcctcag ccttgagtgt actgctgaca actaaactca 3660
caggctgcaa agcagacctc tgatgaagat gcctgttatt tcacatcact gtctttttgt 3720
gtatcatagt ctgcacctta caaatattaa taaatgttcc aataataggt gaaaaaaaaa 3780
a 3781
<210> SEQ ID NO 46
<211> LENGTH: 4072
<212> TYPE: DNA
<213> ORGANISM: Homo sapiens
<400> SEQUENCE: 46
acatgagagg gggagaaata aatatacagt gcttgtcctt agcctttctg tgggcatacc 60
agtgtcagct gcacttgtag gggcccaagt gcctcatgac ccactcggca gccttcctct 120
ccaggatccc caaggctagg aggccaacct actaacaggt gggtgggtat ggtgtgtggt 180
ttcactcagt tcttctcatg gggtttctct gagctccatt cataccagaa agggagcagg 240
agagagagga caagtggatc caacagcctt cgctccaggg gaatcagggc atcgcctcct 300
tttctgggag gacactccct tctgatggtg aatgggaact cccttcctcc tgcagcagcc 360
tgcctgcagc tgtcctggta gaacagtgtg gacattgcag aagctgtcac tgccccagaa 420
agaaagcacc ccagagccaa ggcaaagagt cttgaaagcg ccacaagcag cagctgctga 480
gccatggctg aaggggaaat caccaccttc acagccctga ccgagaagtt taatctgcct 540
ccagggaatt acaagaagcc caaactcctc tactgtagca acgggggcca cttcctgagg 600
atccttccgg atggcacagt ggatgggaca agggacagga gcgaccagca cattcagctg 660
cagctcagtg cggaaagcgt gggggaggtg tatataaaga gtaccgagac tggccagtac 720
ttggccatgg acaccgacgg gcttttatac ggctcacaga caccaaatga ggaatgtttg 780
ttcctggaaa ggctggagga gaaccattac aacacctata tatccaagaa gcatgcagag 840
aagaattggt ttgttggcct caagaagaat gggagctgca aacgcggtcc tcggactcac 900
tatggccaga aagcaatctt gtttctcccc ctgccagtct cttctgatta aagagatctg 960
ttctgggtgt tgaccactcc agagaagttt cgaggggtcc tcacctggtt gacccaaaaa 1020
tgttcccttg accattggct gcgctaaccc ccagcccaca gagcctgaat ttgtaagcaa 1080
cttgcttcta aatgcccagt tcacttcttt gcagagcctt ttacccctgc acagtttaga 1140
acagagggac caaattgctt ctaggagtca actggctggc cagtctgggt ctgggtttgg 1200
atctccaatt gcctcttgca ggctgagtcc ctccatgcaa aagtggggct aaatgaagtg 1260
tgttaagggg tcggctaagt gggacattag taactgcaca ctatttccct ctactgagta 1320
aaccctatct gtgattcccc caaacatctg gcatggctcc cttttgtcct tcctgtgccc 1380
tgcaaatatt agcaaagaag cttcatgcca ggttaggaag gcagcattcc atgaccagaa 1440
acagggacaa agaaatcccc ccttcagaac agaggcattt aaaatggaaa agagagattg 1500
gattttggtg ggtaacttag aaggatggca tctccatgta gaataaatga agaaagggag 1560
gcccagccgc aggaaggcag aataaatcct tgggagtcat taccacgcct tgaccttccc 1620
aaggttactc agcagcagag agccctgggt gacttcaggt ggagagcact agaagtggtt 1680
tcctgataac aagcaaggat atcagagctg ggaaattcat gtggatctgg ggactgagtg 1740
tgggagtgca gagaaagaaa gggaaactgg ctgaggggat accataaaaa gaggatgatt 1800
tcagaaggag aaggaaaaag aaagtaatgc cacacattgt gcttggcccc tggtaagcag 1860
aggctttggg gtcctagccc agtgcttctc caacactgaa gtgcttgcag atcatctggg 1920
gacctggttt gaatggagat tctgattcag tgggttgggg gcagagtttc tgcagttcca 1980
tcaggtcccc cccaggtgca ggtgctgaca atactgctgc cttacccgcc atacattaag 2040
gagcagggtc ctggtcctaa agagttattc aaatgaaggt ggttcgacgc cccgaacctc 2100
acctgacctc aactaaccct taaaaatgca cacctcatga gtctacctga gcattcaggc 2160
agcactgaca atagttatgc ctgtactaag gagcatgatt ttaagaggct ttggcccaat 2220
gcctataaaa tgcccatttc gaagatatac aaaaacatac ttcaaaaatg ttaaaccctt 2280
accaacagct tttcccagga gaccatttgt attaccatta cttgtataaa tacacttcct 2340
gcttaaactt gacccaggtg gctagcaaat tagaaacacc attcatctct aacatatgat 2400
actgatgcca tgtaaaggcc tttaataagt cattgaaatt tactgtgaga ctgtatgttt 2460
taattgcatt taaaaatata tagcttgaaa gcagttaaac tgattagtat tcaggcactg 2520
agaatgatag taataggata caatgtataa gctactcact tatctgatac ttatttacct 2580
ataaaatgag atttttgttt tccactgtgc tattacaaat tttcttttga aagtaggaac 2640
tcttaagcaa tggtaattgt gaataaaaat tgatgagagt gttagctcct gtttcatatg 2700
aaattgaagt aattgttaac taaaaacaat tccttagtaa ctgaactgtc atatttagaa 2760
tggaaggaaa atgacagttt gtgaaagttc aaagcaatag tgcaattgaa gaattgacct 2820
aagtaagctg acattatggt taataatagt attttagatt tgtgcagcaa aataatttca 2880
taactttttt gtttttgtta cttggataag atcaatctgt tttattttag taaatctttg 2940
caggcaagtt agagaaaatg cagtgtggct taacgtctct ttagtatgaa gatttggcca 3000
gaaaaagata cccagagagg aaatctaaga taattataat ggtccatact ttttattgta 3060
tgaatcaaac tcaagcataa cattggccaa ggaaaattaa ataccattgc taacttgtga 3120
aatggaagtc tgtgatttcg gagatgcaaa gcattgtagt aaaaacacca atgtgacctc 3180
gaccatctca gcccagatat cattcatata tctgttcaat gactattaag gtgcctactg 3240
tgtgctaggc actgtactgg atactgggga ccttgtctgt ctggtttgct gctgtatctt 3300
ctcccagggc attatattta tgatgaaaga tgctgtggat tcaattcttt cagtcaagaa 3360
taaacacaga ctttgtaggt tcctgctgaa taaagcaaat cccagaaacc cagattttgg 3420
aagaatcagc aaccccagca taaaataaac ccctatcaaa atgtcagagg acatggcaag 3480
gtaaacttag cattttcaac tttagaaccg ggtcagcttc agggggactg ctttcaaatc 3540
agccaaagag cctgtcagat cttcttagaa ggaagaggtt ggtagttccc tgctctgttt 3600
tgaacatgct ctagtttatt aacctgggga cattcccatt gctgtcttaa gtaagtctca 3660
tagccagctc ctgtcacgtg actctcatat ggattcattt tcgggccagc tctgaacaaa 3720
gcatcatgaa catatgtgct tttggtcgtt tgcaatgtga tggtggtgga ggtaggtatt 3780
ggtttccttg gaaggcatga taagaaagat tcacaatggc caacagtgtg tatgaacaaa 3840
aaactgattg gagcatcagc tagtactgaa ggtccttgct ttgtgtcaga ggcaaaggaa 3900
cccaaggcgc caagtcctca gccttgagtg tactgctgac aactaaactc acaggctgca 3960
aagcagacct ctgatgaaga tgcctgttat ttcacatcac tgtctttttg tgtatcatag 4020
tctgcacctt acaaatatta ataaatgttc caataatagg tgaaaaaaaa aa 4072
<210> SEQ ID NO 47
<211> LENGTH: 4069
<212> TYPE: DNA
<213> ORGANISM: Homo sapiens
<400> SEQUENCE: 47
acatgagagg gggagaaata aatatacagt gcttgtcctt agcctttctg tgggcatacc 60
agtgtcagct gcacttgtag gggcccaagt gcctcatgac ccactcggca gccttcctct 120
ccaggatccc caaggctagg aggccaacct actaacaggt gggtgggtat ggtgtgtggt 180
ttcactcagt tcttctcatg gggtttctct gagctccatt cataccagaa agggagcagg 240
agagagagga caagtggatc caacagcctt cgctccaggg gaatcagggc atcgcctcct 300
tttctgggag gacactccct tctgatggtg aatgggaact cccttcctcc tgcagcagcc 360
tgcctgcagc tgtcctggta gaacagtgtg gacattgcag aagctgtcac tgccccagaa 420
agaaagcacc ccagagccaa ggcaaagagt cttgaaagcg ccacaagcag cagctgctga 480
gccatggctg aaggggaaat caccaccttc acagccctga ccgagaagtt taatctgcct 540
ccagggaatt acaagaagcc caaactcctc tactgtagca acgggggcca cttcctgagg 600
atccttccgg atggcacagt ggatgggaca agggacagga gcgaccagca cattcagctg 660
cagctcagtg cggaaagcgt gggggaggtg tatataaaga gtaccgagac tggccagtac 720
ttggccatgg acaccgacgg gcttttatac ggctcaacac caaatgagga atgtttgttc 780
ctggaaaggc tggaggagaa ccattacaac acctatatat ccaagaagca tgcagagaag 840
aattggtttg ttggcctcaa gaagaatggg agctgcaaac gcggtcctcg gactcactat 900
ggccagaaag caatcttgtt tctccccctg ccagtctctt ctgattaaag agatctgttc 960
tgggtgttga ccactccaga gaagtttcga ggggtcctca cctggttgac ccaaaaatgt 1020
tcccttgacc attggctgcg ctaaccccca gcccacagag cctgaatttg taagcaactt 1080
gcttctaaat gcccagttca cttctttgca gagcctttta cccctgcaca gtttagaaca 1140
gagggaccaa attgcttcta ggagtcaact ggctggccag tctgggtctg ggtttggatc 1200
tccaattgcc tcttgcaggc tgagtccctc catgcaaaag tggggctaaa tgaagtgtgt 1260
taaggggtcg gctaagtggg acattagtaa ctgcacacta tttccctcta ctgagtaaac 1320
cctatctgtg attcccccaa acatctggca tggctccctt ttgtccttcc tgtgccctgc 1380
aaatattagc aaagaagctt catgccaggt taggaaggca gcattccatg accagaaaca 1440
gggacaaaga aatcccccct tcagaacaga ggcatttaaa atggaaaaga gagattggat 1500
tttggtgggt aacttagaag gatggcatct ccatgtagaa taaatgaaga aagggaggcc 1560
cagccgcagg aaggcagaat aaatccttgg gagtcattac cacgccttga ccttcccaag 1620
gttactcagc agcagagagc cctgggtgac ttcaggtgga gagcactaga agtggtttcc 1680
tgataacaag caaggatatc agagctggga aattcatgtg gatctgggga ctgagtgtgg 1740
gagtgcagag aaagaaaggg aaactggctg aggggatacc ataaaaagag gatgatttca 1800
gaaggagaag gaaaaagaaa gtaatgccac acattgtgct tggcccctgg taagcagagg 1860
ctttggggtc ctagcccagt gcttctccaa cactgaagtg cttgcagatc atctggggac 1920
ctggtttgaa tggagattct gattcagtgg gttgggggca gagtttctgc agttccatca 1980
ggtccccccc aggtgcaggt gctgacaata ctgctgcctt acccgccata cattaaggag 2040
cagggtcctg gtcctaaaga gttattcaaa tgaaggtggt tcgacgcccc gaacctcacc 2100
tgacctcaac taacccttaa aaatgcacac ctcatgagtc tacctgagca ttcaggcagc 2160
actgacaata gttatgcctg tactaaggag catgatttta agaggctttg gcccaatgcc 2220
tataaaatgc ccatttcgaa gatatacaaa aacatacttc aaaaatgtta aacccttacc 2280
aacagctttt cccaggagac catttgtatt accattactt gtataaatac acttcctgct 2340
taaacttgac ccaggtggct agcaaattag aaacaccatt catctctaac atatgatact 2400
gatgccatgt aaaggccttt aataagtcat tgaaatttac tgtgagactg tatgttttaa 2460
ttgcatttaa aaatatatag cttgaaagca gttaaactga ttagtattca ggcactgaga 2520
atgatagtaa taggatacaa tgtataagct actcacttat ctgatactta tttacctata 2580
aaatgagatt tttgttttcc actgtgctat tacaaatttt cttttgaaag taggaactct 2640
taagcaatgg taattgtgaa taaaaattga tgagagtgtt agctcctgtt tcatatgaaa 2700
ttgaagtaat tgttaactaa aaacaattcc ttagtaactg aactgtcata tttagaatgg 2760
aaggaaaatg acagtttgtg aaagttcaaa gcaatagtgc aattgaagaa ttgacctaag 2820
taagctgaca ttatggttaa taatagtatt ttagatttgt gcagcaaaat aatttcataa 2880
cttttttgtt tttgttactt ggataagatc aatctgtttt attttagtaa atctttgcag 2940
gcaagttaga gaaaatgcag tgtggcttaa cgtctcttta gtatgaagat ttggccagaa 3000
aaagataccc agagaggaaa tctaagataa ttataatggt ccatactttt tattgtatga 3060
atcaaactca agcataacat tggccaagga aaattaaata ccattgctaa cttgtgaaat 3120
ggaagtctgt gatttcggag atgcaaagca ttgtagtaaa aacaccaatg tgacctcgac 3180
catctcagcc cagatatcat tcatatatct gttcaatgac tattaaggtg cctactgtgt 3240
gctaggcact gtactggata ctggggacct tgtctgtctg gtttgctgct gtatcttctc 3300
ccagggcatt atatttatga tgaaagatgc tgtggattca attctttcag tcaagaataa 3360
acacagactt tgtaggttcc tgctgaataa agcaaatccc agaaacccag attttggaag 3420
aatcagcaac cccagcataa aataaacccc tatcaaaatg tcagaggaca tggcaaggta 3480
aacttagcat tttcaacttt agaaccgggt cagcttcagg gggactgctt tcaaatcagc 3540
caaagagcct gtcagatctt cttagaagga agaggttggt agttccctgc tctgttttga 3600
acatgctcta gtttattaac ctggggacat tcccattgct gtcttaagta agtctcatag 3660
ccagctcctg tcacgtgact ctcatatgga ttcattttcg ggccagctct gaacaaagca 3720
tcatgaacat atgtgctttt ggtcgtttgc aatgtgatgg tggtggaggt aggtattggt 3780
ttccttggaa ggcatgataa gaaagattca caatggccaa cagtgtgtat gaacaaaaaa 3840
ctgattggag catcagctag tactgaaggt ccttgctttg tgtcagaggc aaaggaaccc 3900
aaggcgccaa gtcctcagcc ttgagtgtac tgctgacaac taaactcaca ggctgcaaag 3960
cagacctctg atgaagatgc ctgttatttc acatcactgt ctttttgtgt atcatagtct 4020
gcaccttaca aatattaata aatgttccaa taataggtga aaaaaaaaa 4069
<210> SEQ ID NO 48
<211> LENGTH: 3815
<212> TYPE: DNA
<213> ORGANISM: Homo sapiens
<400> SEQUENCE: 48
agaagtccat tcggctcaca catttgcccc aagacaaacc acgttaaaat aacacccagg 60
agctgcagta gcctggaggt tcagagagcc gggctactct gagaagaaga caccaagtgg 120
attctgcttc ccctgggaca gcactgagcg agtgtggaga gaggtacagc cctcggccta 180
caagctcttt agtcttgaaa gcgccacaag cagcagctgc tgagccatgg ctgaagggga 240
aatcaccacc ttcacagccc tgaccgagaa gtttaatctg cctccaggga attacaagaa 300
gcccaaactc ctctactgta gcaacggggg ccacttcctg aggatccttc cggatggcac 360
agtggatggg acaagggaca ggagcgacca gcacattcag ctgcagctca gtgcggaaag 420
cgtgggggag gtgtatataa agagtaccga gactggccag tacttggcca tggacaccga 480
cgggctttta tacggctcac agacaccaaa tgaggaatgt ttgttcctgg aaaggctgga 540
ggagaaccat tacaacacct atatatccaa gaagcatgca gagaagaatt ggtttgttgg 600
cctcaagaag aatgggagct gcaaacgcgg tcctcggact cactatggcc agaaagcaat 660
cttgtttctc cccctgccag tctcttctga ttaaagagat ctgttctggg tgttgaccac 720
tccagagaag tttcgagggg tcctcacctg gttgacccaa aaatgttccc ttgaccattg 780
gctgcgctaa cccccagccc acagagcctg aatttgtaag caacttgctt ctaaatgccc 840
agttcacttc tttgcagagc cttttacccc tgcacagttt agaacagagg gaccaaattg 900
cttctaggag tcaactggct ggccagtctg ggtctgggtt tggatctcca attgcctctt 960
gcaggctgag tccctccatg caaaagtggg gctaaatgaa gtgtgttaag gggtcggcta 1020
agtgggacat tagtaactgc acactatttc cctctactga gtaaacccta tctgtgattc 1080
ccccaaacat ctggcatggc tcccttttgt ccttcctgtg ccctgcaaat attagcaaag 1140
aagcttcatg ccaggttagg aaggcagcat tccatgacca gaaacaggga caaagaaatc 1200
cccccttcag aacagaggca tttaaaatgg aaaagagaga ttggattttg gtgggtaact 1260
tagaaggatg gcatctccat gtagaataaa tgaagaaagg gaggcccagc cgcaggaagg 1320
cagaataaat ccttgggagt cattaccacg ccttgacctt cccaaggtta ctcagcagca 1380
gagagccctg ggtgacttca ggtggagagc actagaagtg gtttcctgat aacaagcaag 1440
gatatcagag ctgggaaatt catgtggatc tggggactga gtgtgggagt gcagagaaag 1500
aaagggaaac tggctgaggg gataccataa aaagaggatg atttcagaag gagaaggaaa 1560
aagaaagtaa tgccacacat tgtgcttggc ccctggtaag cagaggcttt ggggtcctag 1620
cccagtgctt ctccaacact gaagtgcttg cagatcatct ggggacctgg tttgaatgga 1680
gattctgatt cagtgggttg ggggcagagt ttctgcagtt ccatcaggtc ccccccaggt 1740
gcaggtgctg acaatactgc tgccttaccc gccatacatt aaggagcagg gtcctggtcc 1800
taaagagtta ttcaaatgaa ggtggttcga cgccccgaac ctcacctgac ctcaactaac 1860
ccttaaaaat gcacacctca tgagtctacc tgagcattca ggcagcactg acaatagtta 1920
tgcctgtact aaggagcatg attttaagag gctttggccc aatgcctata aaatgcccat 1980
ttcgaagata tacaaaaaca tacttcaaaa atgttaaacc cttaccaaca gcttttccca 2040
ggagaccatt tgtattacca ttacttgtat aaatacactt cctgcttaaa cttgacccag 2100
gtggctagca aattagaaac accattcatc tctaacatat gatactgatg ccatgtaaag 2160
gcctttaata agtcattgaa atttactgtg agactgtatg ttttaattgc atttaaaaat 2220
atatagcttg aaagcagtta aactgattag tattcaggca ctgagaatga tagtaatagg 2280
atacaatgta taagctactc acttatctga tacttattta cctataaaat gagatttttg 2340
ttttccactg tgctattaca aattttcttt tgaaagtagg aactcttaag caatggtaat 2400
tgtgaataaa aattgatgag agtgttagct cctgtttcat atgaaattga agtaattgtt 2460
aactaaaaac aattccttag taactgaact gtcatattta gaatggaagg aaaatgacag 2520
tttgtgaaag ttcaaagcaa tagtgcaatt gaagaattga cctaagtaag ctgacattat 2580
ggttaataat agtattttag atttgtgcag caaaataatt tcataacttt tttgtttttg 2640
ttacttggat aagatcaatc tgttttattt tagtaaatct ttgcaggcaa gttagagaaa 2700
atgcagtgtg gcttaacgtc tctttagtat gaagatttgg ccagaaaaag atacccagag 2760
aggaaatcta agataattat aatggtccat actttttatt gtatgaatca aactcaagca 2820
taacattggc caaggaaaat taaataccat tgctaacttg tgaaatggaa gtctgtgatt 2880
tcggagatgc aaagcattgt agtaaaaaca ccaatgtgac ctcgaccatc tcagcccaga 2940
tatcattcat atatctgttc aatgactatt aaggtgccta ctgtgtgcta ggcactgtac 3000
tggatactgg ggaccttgtc tgtctggttt gctgctgtat cttctcccag ggcattatat 3060
ttatgatgaa agatgctgtg gattcaattc tttcagtcaa gaataaacac agactttgta 3120
ggttcctgct gaataaagca aatcccagaa acccagattt tggaagaatc agcaacccca 3180
gcataaaata aacccctatc aaaatgtcag aggacatggc aaggtaaact tagcattttc 3240
aactttagaa ccgggtcagc ttcaggggga ctgctttcaa atcagccaaa gagcctgtca 3300
gatcttctta gaaggaagag gttggtagtt ccctgctctg ttttgaacat gctctagttt 3360
attaacctgg ggacattccc attgctgtct taagtaagtc tcatagccag ctcctgtcac 3420
gtgactctca tatggattca ttttcgggcc agctctgaac aaagcatcat gaacatatgt 3480
gcttttggtc gtttgcaatg tgatggtggt ggaggtaggt attggtttcc ttggaaggca 3540
tgataagaaa gattcacaat ggccaacagt gtgtatgaac aaaaaactga ttggagcatc 3600
agctagtact gaaggtcctt gctttgtgtc agaggcaaag gaacccaagg cgccaagtcc 3660
tcagccttga gtgtactgct gacaactaaa ctcacaggct gcaaagcaga cctctgatga 3720
agatgcctgt tatttcacat cactgtcttt ttgtgtatca tagtctgcac cttacaaata 3780
ttaataaatg ttccaataat aggtgaaaaa aaaaa 3815
<210> SEQ ID NO 49
<211> LENGTH: 3813
<212> TYPE: DNA
<213> ORGANISM: Homo sapiens
<400> SEQUENCE: 49
agacatgtaa aaatagtact tctagtttag agactgcaaa aatatgaatg caccatgccg 60
ccacattatc tccattcctc cagtgcccgc ctgacactgg ccctgaatca gggctggagg 120
gggcaggcat ttctcattta ctaaagtgct ggatgcagcc cttgaggttc ggcagaagca 180
gaaagctgcg tcttgaaagc gccacaagca gcagctgctg agccatggct gaaggggaaa 240
tcaccacctt cacagccctg accgagaagt ttaatctgcc tccagggaat tacaagaagc 300
ccaaactcct ctactgtagc aacgggggcc acttcctgag gatccttccg gatggcacag 360
tggatgggac aagggacagg agcgaccagc acattcagct gcagctcagt gcggaaagcg 420
tgggggaggt gtatataaag agtaccgaga ctggccagta cttggccatg gacaccgacg 480
ggcttttata cggctcacag acaccaaatg aggaatgttt gttcctggaa aggctggagg 540
agaaccatta caacacctat atatccaaga agcatgcaga gaagaattgg tttgttggcc 600
tcaagaagaa tgggagctgc aaacgcggtc ctcggactca ctatggccag aaagcaatct 660
tgtttctccc cctgccagtc tcttctgatt aaagagatct gttctgggtg ttgaccactc 720
cagagaagtt tcgaggggtc ctcacctggt tgacccaaaa atgttccctt gaccattggc 780
tgcgctaacc cccagcccac agagcctgaa tttgtaagca acttgcttct aaatgcccag 840
ttcacttctt tgcagagcct tttacccctg cacagtttag aacagaggga ccaaattgct 900
tctaggagtc aactggctgg ccagtctggg tctgggtttg gatctccaat tgcctcttgc 960
aggctgagtc cctccatgca aaagtggggc taaatgaagt gtgttaaggg gtcggctaag 1020
tgggacatta gtaactgcac actatttccc tctactgagt aaaccctatc tgtgattccc 1080
ccaaacatct ggcatggctc ccttttgtcc ttcctgtgcc ctgcaaatat tagcaaagaa 1140
gcttcatgcc aggttaggaa ggcagcattc catgaccaga aacagggaca aagaaatccc 1200
cccttcagaa cagaggcatt taaaatggaa aagagagatt ggattttggt gggtaactta 1260
gaaggatggc atctccatgt agaataaatg aagaaaggga ggcccagccg caggaaggca 1320
gaataaatcc ttgggagtca ttaccacgcc ttgaccttcc caaggttact cagcagcaga 1380
gagccctggg tgacttcagg tggagagcac tagaagtggt ttcctgataa caagcaagga 1440
tatcagagct gggaaattca tgtggatctg gggactgagt gtgggagtgc agagaaagaa 1500
agggaaactg gctgagggga taccataaaa agaggatgat ttcagaagga gaaggaaaaa 1560
gaaagtaatg ccacacattg tgcttggccc ctggtaagca gaggctttgg ggtcctagcc 1620
cagtgcttct ccaacactga agtgcttgca gatcatctgg ggacctggtt tgaatggaga 1680
ttctgattca gtgggttggg ggcagagttt ctgcagttcc atcaggtccc ccccaggtgc 1740
aggtgctgac aatactgctg ccttacccgc catacattaa ggagcagggt cctggtccta 1800
aagagttatt caaatgaagg tggttcgacg ccccgaacct cacctgacct caactaaccc 1860
ttaaaaatgc acacctcatg agtctacctg agcattcagg cagcactgac aatagttatg 1920
cctgtactaa ggagcatgat tttaagaggc tttggcccaa tgcctataaa atgcccattt 1980
cgaagatata caaaaacata cttcaaaaat gttaaaccct taccaacagc ttttcccagg 2040
agaccatttg tattaccatt acttgtataa atacacttcc tgcttaaact tgacccaggt 2100
ggctagcaaa ttagaaacac cattcatctc taacatatga tactgatgcc atgtaaaggc 2160
ctttaataag tcattgaaat ttactgtgag actgtatgtt ttaattgcat ttaaaaatat 2220
atagcttgaa agcagttaaa ctgattagta ttcaggcact gagaatgata gtaataggat 2280
acaatgtata agctactcac ttatctgata cttatttacc tataaaatga gatttttgtt 2340
ttccactgtg ctattacaaa ttttcttttg aaagtaggaa ctcttaagca atggtaattg 2400
tgaataaaaa ttgatgagag tgttagctcc tgtttcatat gaaattgaag taattgttaa 2460
ctaaaaacaa ttccttagta actgaactgt catatttaga atggaaggaa aatgacagtt 2520
tgtgaaagtt caaagcaata gtgcaattga agaattgacc taagtaagct gacattatgg 2580
ttaataatag tattttagat ttgtgcagca aaataatttc ataacttttt tgtttttgtt 2640
acttggataa gatcaatctg ttttatttta gtaaatcttt gcaggcaagt tagagaaaat 2700
gcagtgtggc ttaacgtctc tttagtatga agatttggcc agaaaaagat acccagagag 2760
gaaatctaag ataattataa tggtccatac tttttattgt atgaatcaaa ctcaagcata 2820
acattggcca aggaaaatta aataccattg ctaacttgtg aaatggaagt ctgtgatttc 2880
ggagatgcaa agcattgtag taaaaacacc aatgtgacct cgaccatctc agcccagata 2940
tcattcatat atctgttcaa tgactattaa ggtgcctact gtgtgctagg cactgtactg 3000
gatactgggg accttgtctg tctggtttgc tgctgtatct tctcccaggg cattatattt 3060
atgatgaaag atgctgtgga ttcaattctt tcagtcaaga ataaacacag actttgtagg 3120
ttcctgctga ataaagcaaa tcccagaaac ccagattttg gaagaatcag caaccccagc 3180
ataaaataaa cccctatcaa aatgtcagag gacatggcaa ggtaaactta gcattttcaa 3240
ctttagaacc gggtcagctt cagggggact gctttcaaat cagccaaaga gcctgtcaga 3300
tcttcttaga aggaagaggt tggtagttcc ctgctctgtt ttgaacatgc tctagtttat 3360
taacctgggg acattcccat tgctgtctta agtaagtctc atagccagct cctgtcacgt 3420
gactctcata tggattcatt ttcgggccag ctctgaacaa agcatcatga acatatgtgc 3480
ttttggtcgt ttgcaatgtg atggtggtgg aggtaggtat tggtttcctt ggaaggcatg 3540
ataagaaaga ttcacaatgg ccaacagtgt gtatgaacaa aaaactgatt ggagcatcag 3600
ctagtactga aggtccttgc tttgtgtcag aggcaaagga acccaaggcg ccaagtcctc 3660
agccttgagt gtactgctga caactaaact cacaggctgc aaagcagacc tctgatgaag 3720
atgcctgtta tttcacatca ctgtcttttt gtgtatcata gtctgcacct tacaaatatt 3780
aataaatgtt ccaataatag gtgaaaaaaa aaa 3813
<210> SEQ ID NO 50
<211> LENGTH: 3828
<212> TYPE: DNA
<213> ORGANISM: Homo sapiens
<400> SEQUENCE: 50
agacatgtaa aaatagtact tctagtttag agactgcaaa aatatgaatg caccatgccg 60
ccacattatc tccattcctc cagtgcccgc ctgacactgg ccctgaatca gggctggagg 120
gggcaggcat ttctcattta ctaaagtgct ggatgcagcc cttgaggttc ggcagaagca 180
gaaagctgcg gtgagtctgg ctgtgtcttg aaagcgccac aagcagcagc tgctgagcca 240
tggctgaagg ggaaatcacc accttcacag ccctgaccga gaagtttaat ctgcctccag 300
ggaattacaa gaagcccaaa ctcctctact gtagcaacgg gggccacttc ctgaggatcc 360
ttccggatgg cacagtggat gggacaaggg acaggagcga ccagcacatt cagctgcagc 420
tcagtgcgga aagcgtgggg gaggtgtata taaagagtac cgagactggc cagtacttgg 480
ccatggacac cgacgggctt ttatacggct cacagacacc aaatgaggaa tgtttgttcc 540
tggaaaggct ggaggagaac cattacaaca cctatatatc caagaagcat gcagagaaga 600
attggtttgt tggcctcaag aagaatggga gctgcaaacg cggtcctcgg actcactatg 660
gccagaaagc aatcttgttt ctccccctgc cagtctcttc tgattaaaga gatctgttct 720
gggtgttgac cactccagag aagtttcgag gggtcctcac ctggttgacc caaaaatgtt 780
cccttgacca ttggctgcgc taacccccag cccacagagc ctgaatttgt aagcaacttg 840
cttctaaatg cccagttcac ttctttgcag agccttttac ccctgcacag tttagaacag 900
agggaccaaa ttgcttctag gagtcaactg gctggccagt ctgggtctgg gtttggatct 960
ccaattgcct cttgcaggct gagtccctcc atgcaaaagt ggggctaaat gaagtgtgtt 1020
aaggggtcgg ctaagtggga cattagtaac tgcacactat ttccctctac tgagtaaacc 1080
ctatctgtga ttcccccaaa catctggcat ggctcccttt tgtccttcct gtgccctgca 1140
aatattagca aagaagcttc atgccaggtt aggaaggcag cattccatga ccagaaacag 1200
ggacaaagaa atcccccctt cagaacagag gcatttaaaa tggaaaagag agattggatt 1260
ttggtgggta acttagaagg atggcatctc catgtagaat aaatgaagaa agggaggccc 1320
agccgcagga aggcagaata aatccttggg agtcattacc acgccttgac cttcccaagg 1380
ttactcagca gcagagagcc ctgggtgact tcaggtggag agcactagaa gtggtttcct 1440
gataacaagc aaggatatca gagctgggaa attcatgtgg atctggggac tgagtgtggg 1500
agtgcagaga aagaaaggga aactggctga ggggatacca taaaaagagg atgatttcag 1560
aaggagaagg aaaaagaaag taatgccaca cattgtgctt ggcccctggt aagcagaggc 1620
tttggggtcc tagcccagtg cttctccaac actgaagtgc ttgcagatca tctggggacc 1680
tggtttgaat ggagattctg attcagtggg ttgggggcag agtttctgca gttccatcag 1740
gtccccccca ggtgcaggtg ctgacaatac tgctgcctta cccgccatac attaaggagc 1800
agggtcctgg tcctaaagag ttattcaaat gaaggtggtt cgacgccccg aacctcacct 1860
gacctcaact aacccttaaa aatgcacacc tcatgagtct acctgagcat tcaggcagca 1920
ctgacaatag ttatgcctgt actaaggagc atgattttaa gaggctttgg cccaatgcct 1980
ataaaatgcc catttcgaag atatacaaaa acatacttca aaaatgttaa acccttacca 2040
acagcttttc ccaggagacc atttgtatta ccattacttg tataaataca cttcctgctt 2100
aaacttgacc caggtggcta gcaaattaga aacaccattc atctctaaca tatgatactg 2160
atgccatgta aaggccttta ataagtcatt gaaatttact gtgagactgt atgttttaat 2220
tgcatttaaa aatatatagc ttgaaagcag ttaaactgat tagtattcag gcactgagaa 2280
tgatagtaat aggatacaat gtataagcta ctcacttatc tgatacttat ttacctataa 2340
aatgagattt ttgttttcca ctgtgctatt acaaattttc ttttgaaagt aggaactctt 2400
aagcaatggt aattgtgaat aaaaattgat gagagtgtta gctcctgttt catatgaaat 2460
tgaagtaatt gttaactaaa aacaattcct tagtaactga actgtcatat ttagaatgga 2520
aggaaaatga cagtttgtga aagttcaaag caatagtgca attgaagaat tgacctaagt 2580
aagctgacat tatggttaat aatagtattt tagatttgtg cagcaaaata atttcataac 2640
ttttttgttt ttgttacttg gataagatca atctgtttta ttttagtaaa tctttgcagg 2700
caagttagag aaaatgcagt gtggcttaac gtctctttag tatgaagatt tggccagaaa 2760
aagataccca gagaggaaat ctaagataat tataatggtc catacttttt attgtatgaa 2820
tcaaactcaa gcataacatt ggccaaggaa aattaaatac cattgctaac ttgtgaaatg 2880
gaagtctgtg atttcggaga tgcaaagcat tgtagtaaaa acaccaatgt gacctcgacc 2940
atctcagccc agatatcatt catatatctg ttcaatgact attaaggtgc ctactgtgtg 3000
ctaggcactg tactggatac tggggacctt gtctgtctgg tttgctgctg tatcttctcc 3060
cagggcatta tatttatgat gaaagatgct gtggattcaa ttctttcagt caagaataaa 3120
cacagacttt gtaggttcct gctgaataaa gcaaatccca gaaacccaga ttttggaaga 3180
atcagcaacc ccagcataaa ataaacccct atcaaaatgt cagaggacat ggcaaggtaa 3240
acttagcatt ttcaacttta gaaccgggtc agcttcaggg ggactgcttt caaatcagcc 3300
aaagagcctg tcagatcttc ttagaaggaa gaggttggta gttccctgct ctgttttgaa 3360
catgctctag tttattaacc tggggacatt cccattgctg tcttaagtaa gtctcatagc 3420
cagctcctgt cacgtgactc tcatatggat tcattttcgg gccagctctg aacaaagcat 3480
catgaacata tgtgcttttg gtcgtttgca atgtgatggt ggtggaggta ggtattggtt 3540
tccttggaag gcatgataag aaagattcac aatggccaac agtgtgtatg aacaaaaaac 3600
tgattggagc atcagctagt actgaaggtc cttgctttgt gtcagaggca aaggaaccca 3660
aggcgccaag tcctcagcct tgagtgtact gctgacaact aaactcacag gctgcaaagc 3720
agacctctga tgaagatgcc tgttatttca catcactgtc tttttgtgta tcatagtctg 3780
caccttacaa atattaataa atgttccaat aataggtgaa aaaaaaaa 3828
<210> SEQ ID NO 51
<211> LENGTH: 3812
<212> TYPE: DNA
<213> ORGANISM: Homo sapiens
<400> SEQUENCE: 51
tcaaaatgac ctaagatatt ctgagtcaga gaaaacaaaa ggaacagctt aaagagagca 60
ccaactcagt gaggcaacca ggcagtgggg ccggctggcc agactcttgg gggattcctt 120
agtgagtgag ttcactgctc aaagaagggc tttgccactt ctgcagggaa gccagccacg 180
ggccagcagt cttgaaagcg ccacaagcag cagctgctga gccatggctg aaggggaaat 240
caccaccttc acagccctga ccgagaagtt taatctgcct ccagggaatt acaagaagcc 300
caaactcctc tactgtagca acgggggcca cttcctgagg atccttccgg atggcacagt 360
ggatgggaca agggacagga gcgaccagca cattcagctg cagctcagtg cggaaagcgt 420
gggggaggtg tatataaaga gtaccgagac tggccagtac ttggccatgg acaccgacgg 480
gcttttatac ggctcacaga caccaaatga ggaatgtttg ttcctggaaa ggctggagga 540
gaaccattac aacacctata tatccaagaa gcatgcagag aagaattggt ttgttggcct 600
caagaagaat gggagctgca aacgcggtcc tcggactcac tatggccaga aagcaatctt 660
gtttctcccc ctgccagtct cttctgatta aagagatctg ttctgggtgt tgaccactcc 720
agagaagttt cgaggggtcc tcacctggtt gacccaaaaa tgttcccttg accattggct 780
gcgctaaccc ccagcccaca gagcctgaat ttgtaagcaa cttgcttcta aatgcccagt 840
tcacttcttt gcagagcctt ttacccctgc acagtttaga acagagggac caaattgctt 900
ctaggagtca actggctggc cagtctgggt ctgggtttgg atctccaatt gcctcttgca 960
ggctgagtcc ctccatgcaa aagtggggct aaatgaagtg tgttaagggg tcggctaagt 1020
gggacattag taactgcaca ctatttccct ctactgagta aaccctatct gtgattcccc 1080
caaacatctg gcatggctcc cttttgtcct tcctgtgccc tgcaaatatt agcaaagaag 1140
cttcatgcca ggttaggaag gcagcattcc atgaccagaa acagggacaa agaaatcccc 1200
ccttcagaac agaggcattt aaaatggaaa agagagattg gattttggtg ggtaacttag 1260
aaggatggca tctccatgta gaataaatga agaaagggag gcccagccgc aggaaggcag 1320
aataaatcct tgggagtcat taccacgcct tgaccttccc aaggttactc agcagcagag 1380
agccctgggt gacttcaggt ggagagcact agaagtggtt tcctgataac aagcaaggat 1440
atcagagctg ggaaattcat gtggatctgg ggactgagtg tgggagtgca gagaaagaaa 1500
gggaaactgg ctgaggggat accataaaaa gaggatgatt tcagaaggag aaggaaaaag 1560
aaagtaatgc cacacattgt gcttggcccc tggtaagcag aggctttggg gtcctagccc 1620
agtgcttctc caacactgaa gtgcttgcag atcatctggg gacctggttt gaatggagat 1680
tctgattcag tgggttgggg gcagagtttc tgcagttcca tcaggtcccc cccaggtgca 1740
ggtgctgaca atactgctgc cttacccgcc atacattaag gagcagggtc ctggtcctaa 1800
agagttattc aaatgaaggt ggttcgacgc cccgaacctc acctgacctc aactaaccct 1860
taaaaatgca cacctcatga gtctacctga gcattcaggc agcactgaca atagttatgc 1920
ctgtactaag gagcatgatt ttaagaggct ttggcccaat gcctataaaa tgcccatttc 1980
gaagatatac aaaaacatac ttcaaaaatg ttaaaccctt accaacagct tttcccagga 2040
gaccatttgt attaccatta cttgtataaa tacacttcct gcttaaactt gacccaggtg 2100
gctagcaaat tagaaacacc attcatctct aacatatgat actgatgcca tgtaaaggcc 2160
tttaataagt cattgaaatt tactgtgaga ctgtatgttt taattgcatt taaaaatata 2220
tagcttgaaa gcagttaaac tgattagtat tcaggcactg agaatgatag taataggata 2280
caatgtataa gctactcact tatctgatac ttatttacct ataaaatgag atttttgttt 2340
tccactgtgc tattacaaat tttcttttga aagtaggaac tcttaagcaa tggtaattgt 2400
gaataaaaat tgatgagagt gttagctcct gtttcatatg aaattgaagt aattgttaac 2460
taaaaacaat tccttagtaa ctgaactgtc atatttagaa tggaaggaaa atgacagttt 2520
gtgaaagttc aaagcaatag tgcaattgaa gaattgacct aagtaagctg acattatggt 2580
taataatagt attttagatt tgtgcagcaa aataatttca taactttttt gtttttgtta 2640
cttggataag atcaatctgt tttattttag taaatctttg caggcaagtt agagaaaatg 2700
cagtgtggct taacgtctct ttagtatgaa gatttggcca gaaaaagata cccagagagg 2760
aaatctaaga taattataat ggtccatact ttttattgta tgaatcaaac tcaagcataa 2820
cattggccaa ggaaaattaa ataccattgc taacttgtga aatggaagtc tgtgatttcg 2880
gagatgcaaa gcattgtagt aaaaacacca atgtgacctc gaccatctca gcccagatat 2940
cattcatata tctgttcaat gactattaag gtgcctactg tgtgctaggc actgtactgg 3000
atactgggga ccttgtctgt ctggtttgct gctgtatctt ctcccagggc attatattta 3060
tgatgaaaga tgctgtggat tcaattcttt cagtcaagaa taaacacaga ctttgtaggt 3120
tcctgctgaa taaagcaaat cccagaaacc cagattttgg aagaatcagc aaccccagca 3180
taaaataaac ccctatcaaa atgtcagagg acatggcaag gtaaacttag cattttcaac 3240
tttagaaccg ggtcagcttc agggggactg ctttcaaatc agccaaagag cctgtcagat 3300
cttcttagaa ggaagaggtt ggtagttccc tgctctgttt tgaacatgct ctagtttatt 3360
aacctgggga cattcccatt gctgtcttaa gtaagtctca tagccagctc ctgtcacgtg 3420
actctcatat ggattcattt tcgggccagc tctgaacaaa gcatcatgaa catatgtgct 3480
tttggtcgtt tgcaatgtga tggtggtgga ggtaggtatt ggtttccttg gaaggcatga 3540
taagaaagat tcacaatggc caacagtgtg tatgaacaaa aaactgattg gagcatcagc 3600
tagtactgaa ggtccttgct ttgtgtcaga ggcaaaggaa cccaaggcgc caagtcctca 3660
gccttgagtg tactgctgac aactaaactc acaggctgca aagcagacct ctgatgaaga 3720
tgcctgttat ttcacatcac tgtctttttg tgtatcatag tctgcacctt acaaatatta 3780
ataaatgttc caataatagg tgaaaaaaaa aa 3812
<210> SEQ ID NO 52
<211> LENGTH: 3810
<212> TYPE: DNA
<213> ORGANISM: Homo sapiens
<400> SEQUENCE: 52
agacatgtaa aaatagtact tctagtttag agactgcaaa aatatgaatg caccatgccg 60
ccacattatc tccattcctc cagtgcccgc ctgacactgg ccctgaatca gggctggagg 120
gggcaggcat ttctcattta ctaaagtgct ggatgcagcc cttgaggttc ggcagaagca 180
gaaagctgcg tcttgaaagc gccacaagca gcagctgctg agccatggct gaaggggaaa 240
tcaccacctt cacagccctg accgagaagt ttaatctgcc tccagggaat tacaagaagc 300
ccaaactcct ctactgtagc aacgggggcc acttcctgag gatccttccg gatggcacag 360
tggatgggac aagggacagg agcgaccagc acattcagct gcagctcagt gcggaaagcg 420
tgggggaggt gtatataaag agtaccgaga ctggccagta cttggccatg gacaccgacg 480
ggcttttata cggctcaaca ccaaatgagg aatgtttgtt cctggaaagg ctggaggaga 540
accattacaa cacctatata tccaagaagc atgcagagaa gaattggttt gttggcctca 600
agaagaatgg gagctgcaaa cgcggtcctc ggactcacta tggccagaaa gcaatcttgt 660
ttctccccct gccagtctct tctgattaaa gagatctgtt ctgggtgttg accactccag 720
agaagtttcg aggggtcctc acctggttga cccaaaaatg ttcccttgac cattggctgc 780
gctaaccccc agcccacaga gcctgaattt gtaagcaact tgcttctaaa tgcccagttc 840
acttctttgc agagcctttt acccctgcac agtttagaac agagggacca aattgcttct 900
aggagtcaac tggctggcca gtctgggtct gggtttggat ctccaattgc ctcttgcagg 960
ctgagtccct ccatgcaaaa gtggggctaa atgaagtgtg ttaaggggtc ggctaagtgg 1020
gacattagta actgcacact atttccctct actgagtaaa ccctatctgt gattccccca 1080
aacatctggc atggctccct tttgtccttc ctgtgccctg caaatattag caaagaagct 1140
tcatgccagg ttaggaaggc agcattccat gaccagaaac agggacaaag aaatcccccc 1200
ttcagaacag aggcatttaa aatggaaaag agagattgga ttttggtggg taacttagaa 1260
ggatggcatc tccatgtaga ataaatgaag aaagggaggc ccagccgcag gaaggcagaa 1320
taaatccttg ggagtcatta ccacgccttg accttcccaa ggttactcag cagcagagag 1380
ccctgggtga cttcaggtgg agagcactag aagtggtttc ctgataacaa gcaaggatat 1440
cagagctggg aaattcatgt ggatctgggg actgagtgtg ggagtgcaga gaaagaaagg 1500
gaaactggct gaggggatac cataaaaaga ggatgatttc agaaggagaa ggaaaaagaa 1560
agtaatgcca cacattgtgc ttggcccctg gtaagcagag gctttggggt cctagcccag 1620
tgcttctcca acactgaagt gcttgcagat catctgggga cctggtttga atggagattc 1680
tgattcagtg ggttgggggc agagtttctg cagttccatc aggtcccccc caggtgcagg 1740
tgctgacaat actgctgcct tacccgccat acattaagga gcagggtcct ggtcctaaag 1800
agttattcaa atgaaggtgg ttcgacgccc cgaacctcac ctgacctcaa ctaaccctta 1860
aaaatgcaca cctcatgagt ctacctgagc attcaggcag cactgacaat agttatgcct 1920
gtactaagga gcatgatttt aagaggcttt ggcccaatgc ctataaaatg cccatttcga 1980
agatatacaa aaacatactt caaaaatgtt aaacccttac caacagcttt tcccaggaga 2040
ccatttgtat taccattact tgtataaata cacttcctgc ttaaacttga cccaggtggc 2100
tagcaaatta gaaacaccat tcatctctaa catatgatac tgatgccatg taaaggcctt 2160
taataagtca ttgaaattta ctgtgagact gtatgtttta attgcattta aaaatatata 2220
gcttgaaagc agttaaactg attagtattc aggcactgag aatgatagta ataggataca 2280
atgtataagc tactcactta tctgatactt atttacctat aaaatgagat ttttgttttc 2340
cactgtgcta ttacaaattt tcttttgaaa gtaggaactc ttaagcaatg gtaattgtga 2400
ataaaaattg atgagagtgt tagctcctgt ttcatatgaa attgaagtaa ttgttaacta 2460
aaaacaattc cttagtaact gaactgtcat atttagaatg gaaggaaaat gacagtttgt 2520
gaaagttcaa agcaatagtg caattgaaga attgacctaa gtaagctgac attatggtta 2580
ataatagtat tttagatttg tgcagcaaaa taatttcata acttttttgt ttttgttact 2640
tggataagat caatctgttt tattttagta aatctttgca ggcaagttag agaaaatgca 2700
gtgtggctta acgtctcttt agtatgaaga tttggccaga aaaagatacc cagagaggaa 2760
atctaagata attataatgg tccatacttt ttattgtatg aatcaaactc aagcataaca 2820
ttggccaagg aaaattaaat accattgcta acttgtgaaa tggaagtctg tgatttcgga 2880
gatgcaaagc attgtagtaa aaacaccaat gtgacctcga ccatctcagc ccagatatca 2940
ttcatatatc tgttcaatga ctattaaggt gcctactgtg tgctaggcac tgtactggat 3000
actggggacc ttgtctgtct ggtttgctgc tgtatcttct cccagggcat tatatttatg 3060
atgaaagatg ctgtggattc aattctttca gtcaagaata aacacagact ttgtaggttc 3120
ctgctgaata aagcaaatcc cagaaaccca gattttggaa gaatcagcaa ccccagcata 3180
aaataaaccc ctatcaaaat gtcagaggac atggcaaggt aaacttagca ttttcaactt 3240
tagaaccggg tcagcttcag ggggactgct ttcaaatcag ccaaagagcc tgtcagatct 3300
tcttagaagg aagaggttgg tagttccctg ctctgttttg aacatgctct agtttattaa 3360
cctggggaca ttcccattgc tgtcttaagt aagtctcata gccagctcct gtcacgtgac 3420
tctcatatgg attcattttc gggccagctc tgaacaaagc atcatgaaca tatgtgcttt 3480
tggtcgtttg caatgtgatg gtggtggagg taggtattgg tttccttgga aggcatgata 3540
agaaagattc acaatggcca acagtgtgta tgaacaaaaa actgattgga gcatcagcta 3600
gtactgaagg tccttgcttt gtgtcagagg caaaggaacc caaggcgcca agtcctcagc 3660
cttgagtgta ctgctgacaa ctaaactcac aggctgcaaa gcagacctct gatgaagatg 3720
cctgttattt cacatcactg tctttttgtg tatcatagtc tgcaccttac aaatattaat 3780
aaatgttcca ataataggtg aaaaaaaaaa 3810
<210> SEQ ID NO 53
<211> LENGTH: 3679
<212> TYPE: DNA
<213> ORGANISM: Homo sapiens
<400> SEQUENCE: 53
aaaaagagag agagaaaaaa tactgttggc agcagcacaa tgtttgggct aagacctggt 60
cttgaaagcg ccacaagcag cagctgctga gccatggctg aaggggaaat caccaccttc 120
acagccctga ccgagaagtt taatctgcct ccagggaatt acaagaagcc caaactcctc 180
tactgtagca acgggggcca cttcctgagg atccttccgg atggcacagt ggatgggaca 240
agggacagga gcgaccagca cattcagctg cagctcagtg cggaaagcgt gggggaggtg 300
tatataaaga gtaccgagac tggccagtac ttggccatgg acaccgacgg gcttttatac 360
ggctcaacac caaatgagga atgtttgttc ctggaaaggc tggaggagaa ccattacaac 420
acctatatat ccaagaagca tgcagagaag aattggtttg ttggcctcaa gaagaatggg 480
agctgcaaac gcggtcctcg gactcactat ggccagaaag caatcttgtt tctccccctg 540
ccagtctctt ctgattaaag agatctgttc tgggtgttga ccactccaga gaagtttcga 600
ggggtcctca cctggttgac ccaaaaatgt tcccttgacc attggctgcg ctaaccccca 660
gcccacagag cctgaatttg taagcaactt gcttctaaat gcccagttca cttctttgca 720
gagcctttta cccctgcaca gtttagaaca gagggaccaa attgcttcta ggagtcaact 780
ggctggccag tctgggtctg ggtttggatc tccaattgcc tcttgcaggc tgagtccctc 840
catgcaaaag tggggctaaa tgaagtgtgt taaggggtcg gctaagtggg acattagtaa 900
ctgcacacta tttccctcta ctgagtaaac cctatctgtg attcccccaa acatctggca 960
tggctccctt ttgtccttcc tgtgccctgc aaatattagc aaagaagctt catgccaggt 1020
taggaaggca gcattccatg accagaaaca gggacaaaga aatcccccct tcagaacaga 1080
ggcatttaaa atggaaaaga gagattggat tttggtgggt aacttagaag gatggcatct 1140
ccatgtagaa taaatgaaga aagggaggcc cagccgcagg aaggcagaat aaatccttgg 1200
gagtcattac cacgccttga ccttcccaag gttactcagc agcagagagc cctgggtgac 1260
ttcaggtgga gagcactaga agtggtttcc tgataacaag caaggatatc agagctggga 1320
aattcatgtg gatctgggga ctgagtgtgg gagtgcagag aaagaaaggg aaactggctg 1380
aggggatacc ataaaaagag gatgatttca gaaggagaag gaaaaagaaa gtaatgccac 1440
acattgtgct tggcccctgg taagcagagg ctttggggtc ctagcccagt gcttctccaa 1500
cactgaagtg cttgcagatc atctggggac ctggtttgaa tggagattct gattcagtgg 1560
gttgggggca gagtttctgc agttccatca ggtccccccc aggtgcaggt gctgacaata 1620
ctgctgcctt acccgccata cattaaggag cagggtcctg gtcctaaaga gttattcaaa 1680
tgaaggtggt tcgacgcccc gaacctcacc tgacctcaac taacccttaa aaatgcacac 1740
ctcatgagtc tacctgagca ttcaggcagc actgacaata gttatgcctg tactaaggag 1800
catgatttta agaggctttg gcccaatgcc tataaaatgc ccatttcgaa gatatacaaa 1860
aacatacttc aaaaatgtta aacccttacc aacagctttt cccaggagac catttgtatt 1920
accattactt gtataaatac acttcctgct taaacttgac ccaggtggct agcaaattag 1980
aaacaccatt catctctaac atatgatact gatgccatgt aaaggccttt aataagtcat 2040
tgaaatttac tgtgagactg tatgttttaa ttgcatttaa aaatatatag cttgaaagca 2100
gttaaactga ttagtattca ggcactgaga atgatagtaa taggatacaa tgtataagct 2160
actcacttat ctgatactta tttacctata aaatgagatt tttgttttcc actgtgctat 2220
tacaaatttt cttttgaaag taggaactct taagcaatgg taattgtgaa taaaaattga 2280
tgagagtgtt agctcctgtt tcatatgaaa ttgaagtaat tgttaactaa aaacaattcc 2340
ttagtaactg aactgtcata tttagaatgg aaggaaaatg acagtttgtg aaagttcaaa 2400
gcaatagtgc aattgaagaa ttgacctaag taagctgaca ttatggttaa taatagtatt 2460
ttagatttgt gcagcaaaat aatttcataa cttttttgtt tttgttactt ggataagatc 2520
aatctgtttt attttagtaa atctttgcag gcaagttaga gaaaatgcag tgtggcttaa 2580
cgtctcttta gtatgaagat ttggccagaa aaagataccc agagaggaaa tctaagataa 2640
ttataatggt ccatactttt tattgtatga atcaaactca agcataacat tggccaagga 2700
aaattaaata ccattgctaa cttgtgaaat ggaagtctgt gatttcggag atgcaaagca 2760
ttgtagtaaa aacaccaatg tgacctcgac catctcagcc cagatatcat tcatatatct 2820
gttcaatgac tattaaggtg cctactgtgt gctaggcact gtactggata ctggggacct 2880
tgtctgtctg gtttgctgct gtatcttctc ccagggcatt atatttatga tgaaagatgc 2940
tgtggattca attctttcag tcaagaataa acacagactt tgtaggttcc tgctgaataa 3000
agcaaatccc agaaacccag attttggaag aatcagcaac cccagcataa aataaacccc 3060
tatcaaaatg tcagaggaca tggcaaggta aacttagcat tttcaacttt agaaccgggt 3120
cagcttcagg gggactgctt tcaaatcagc caaagagcct gtcagatctt cttagaagga 3180
agaggttggt agttccctgc tctgttttga acatgctcta gtttattaac ctggggacat 3240
tcccattgct gtcttaagta agtctcatag ccagctcctg tcacgtgact ctcatatgga 3300
ttcattttcg ggccagctct gaacaaagca tcatgaacat atgtgctttt ggtcgtttgc 3360
aatgtgatgg tggtggaggt aggtattggt ttccttggaa ggcatgataa gaaagattca 3420
caatggccaa cagtgtgtat gaacaaaaaa ctgattggag catcagctag tactgaaggt 3480
ccttgctttg tgtcagaggc aaaggaaccc aaggcgccaa gtcctcagcc ttgagtgtac 3540
tgctgacaac taaactcaca ggctgcaaag cagacctctg atgaagatgc ctgttatttc 3600
acatcactgt ctttttgtgt atcatagtct gcaccttaca aatattaata aatgttccaa 3660
taataggtga aaaaaaaaa 3679
<210> SEQ ID NO 54
<211> LENGTH: 6774
<212> TYPE: DNA
<213> ORGANISM: Homo sapiens
<400> SEQUENCE: 54
cggccccaga aaacccgagc gagtaggggg cggcgcgcag gagggaggag aactgggggc 60
gcgggaggct ggtgggtgtg gggggtggag atgtagaaga tgtgacgccg cggcccggcg 120
ggtgccagat tagcggacgc ggtgcccgcg gttgcaacgg gatcccgggc gctgcagctt 180
gggaggcggc tctccccagg cggcgtccgc ggagacaccc atccgtgaac cccaggtccc 240
gggccgccgg ctcgccgcgc accaggggcc ggcggacaga agagcggccg agcggctcga 300
ggctggggga ccgcgggcgc ggccgcgcgc tgccgggcgg gaggctgggg ggccggggcc 360
ggggccgtgc cccggagcgg gtcggaggcc ggggccgggg ccgggggacg gcggctcccc 420
gcgcggctcc agcggctcgg ggatcccggc cgggccccgc agggaccatg gcagccggga 480
gcatcaccac gctgcccgcc ttgcccgagg atggcggcag cggcgccttc ccgcccggcc 540
acttcaagga ccccaagcgg ctgtactgca aaaacggggg cttcttcctg cgcatccacc 600
ccgacggccg agttgacggg gtccgggaga agagcgaccc tcacatcaag ctacaacttc 660
aagcagaaga gagaggagtt gtgtctatca aaggagtgtg tgctaaccgt tacctggcta 720
tgaaggaaga tggaagatta ctggcttcta aatgtgttac ggatgagtgt ttcttttttg 780
aacgattgga atctaataac tacaatactt accggtcaag gaaatacacc agttggtatg 840
tggcactgaa acgaactggg cagtataaac ttggatccaa aacaggacct gggcagaaag 900
ctatactttt tcttccaatg tctgctaaga gctgatttta atggccacat ctaatctcat 960
ttcacatgaa agaagaagta tattttagaa atttgttaat gagagtaaaa gaaaataaat 1020
gtgtatagct cagtttggat aattggtcaa acaatttttt atccagtagt aaaatatgta 1080
accattgtcc cagtaaagaa aaataacaaa agttgtaaaa tgtatattct cccttttata 1140
ttgcatctgc tgttacccag tgaagcttac ctagagcaat gatctttttc acgcatttgc 1200
tttattcgaa aagaggcttt taaaatgtgc atgtttagaa acaaaatttc ttcatggaaa 1260
tcatatacat tagaaaatca cagtcagatg tttaatcaat ccaaaatgtc cactatttct 1320
tatgtcattc gttagtctac atgtttctaa acatataaat gtgaatttaa tcaattcctt 1380
tcatagtttt ataattctct ggcagttcct tatgatagag tttataaaac agtcctgtgt 1440
aaactgctgg aagttcttcc acagtcaggt caattttgtc aaacccttct ctgtacccat 1500
acagcagcag cctagcaact ctgctggtga tgggagttgt attttcagtc ttcgccaggt 1560
cattgagatc catccactca catcttaagc attcttcctg gcaaaaattt atggtgaatg 1620
aatatggctt taggcggcag atgatataca tatctgactt cccaaaagct ccaggatttg 1680
tgtgctgttg ccgaatactc aggacggacc tgaattctga ttttatacca gtctcttcaa 1740
aaacttctcg aaccgctgtg tctcctacgt aaaaaaagag atgtacaaat caataataat 1800
tacactttta gaaactgtat catcaaagat tttcagttaa agtagcatta tgtaaaggct 1860
caaaacatta ccctaacaaa gtaaagtttt caatacaaat tctttgcctt gtggatatca 1920
agaaatccca aaatattttc ttaccactgt aaattcaaga agcttttgaa atgctgaata 1980
tttctttggc tgctacttgg aggcttatct acctgtacat ttttggggtc agctcttttt 2040
aacttcttgc tgctcttttt cccaaaaggt aaaaatatag attgaaaagt taaaacattt 2100
tgcatggctg cagttccttt gtttcttgag ataagattcc aaagaactta gattcatttc 2160
ttcaacaccg aaatgctgga ggtgtttgat cagttttcaa gaaacttgga atataaataa 2220
ttttataatt caacaaaggt tttcacattt tataaggttg atttttcaat taaatgcaaa 2280
tttgtgtggc aggattttta ttgccattaa catatttttg tggctgcttt ttctacacat 2340
ccagatggtc cctctaactg ggctttctct aattttgtga tgttctgtca ttgtctccca 2400
aagtatttag gagaagccct ttaaaaagct gccttcctct accactttgc tggaaagctt 2460
cacaattgtc acagacaaag atttttgttc caatactcgt tttgcctcta tttttcttgt 2520
ttgtcaaata gtaaatgata tttgcccttg cagtaattct actggtgaaa aacatgcaaa 2580
gaagaggaag tcacagaaac atgtctcaat tcccatgtgc tgtgactgta gactgtctta 2640
ccatagactg tcttacccat cccctggata tgctcttgtt ttttccctct aatagctatg 2700
gaaagatgca tagaaagagt ataatgtttt aaaacataag gcattcgtct gccatttttc 2760
aattacatgc tgacttccct tacaattgag atttgcccat aggttaaaca tggttagaaa 2820
caactgaaag cataaaagaa aaatctaggc cgggtgcagt ggctcatgcc tatattccct 2880
gcactttggg aggccaaagc aggaggatcg cttgagccca ggagttcaag accaacctgg 2940
tgaaaccccg tctctacaaa aaaacacaaa aaatagccag gcatggtggc gtgtacatgt 3000
ggtctcagat acttgggagg ctgaggtggg agggttgatc acttgaggct gagaggtcaa 3060
ggttgcagtg agccataatc gtgccactgc agtccagcct aggcaacaga gtgagacttt 3120
gtctcaaaaa aagagaaatt ttccttaata agaaaagtaa tttttactct gatgtgcaat 3180
acatttgtta ttaaatttat tatttaagat ggtagcacta gtcttaaatt gtataaaata 3240
tcccctaaca tgtttaaatg tccattttta ttcattatgc tttgaaaaat aattatgggg 3300
aaatacatgt ttgttattaa atttattatt aaagatagta gcactagtct taaatttgat 3360
ataacatctc ctaacttgtt taaatgtcca tttttattct ttatgtttga aaataaatta 3420
tggggatcct atttagctct tagtaccact aatcaaaagt tcggcatgta gctcatgatc 3480
tatgctgttt ctatgtcgtg gaagcaccgg atgggggtag tgagcaaatc tgccctgctc 3540
agcagtcacc atagcagctg actgaaaatc agcactgcct gagtagtttt gatcagttta 3600
acttgaatca ctaactgact gaaaattgaa tgggcaaata agtgcttttg tctccagagt 3660
atgcgggaga cccttccacc tcaagatgga tatttcttcc ccaaggattt caagatgaat 3720
tgaaattttt aatcaagata gtgtgcttta ttctgttgta ttttttatta ttttaatata 3780
ctgtaagcca aactgaaata acatttgctg ttttataggt ttgaagaaca taggaaaaac 3840
taagaggttt tgtttttatt tttgctgatg aagagatatg tttaaatatg ttgtattgtt 3900
ttgtttagtt acaggacaat aatgaaatgg agtttatatt tgttatttct attttgttat 3960
atttaataat agaattagat tgaaataaaa tataatggga aataatctgc agaatgtggg 4020
ttttcctggt gtttccctct gactctagtg cactgatgat ctctgataag gctcagctgc 4080
tttatagttc tctggctaat gcagcagata ctcttcctgc cagtggtaat acgatttttt 4140
aagaaggcag tttgtcaatt ttaatcttgt ggataccttt atactcttag ggtattattt 4200
tatacaaaag ccttgaggat tgcattctat tttctatatg accctcttga tatttaaaaa 4260
acactatgga taacaattct tcatttacct agtattatga aagaatgaag gagttcaaac 4320
aaatgtgttt cccagttaac tagggtttac tgtttgagcc aatataaatg tttaactgtt 4380
tgtgatggca gtattcctaa agtacattgc atgttttcct aaatacagag tttaaataat 4440
ttcagtaatt cttagatgat tcagcttcat cattaagaat atcttttgtt ttatgttgag 4500
ttagaaatgc cttcatatag acatagtctt tcagacctct actgtcagtt ttcatttcta 4560
gctgctttca gggttttatg aattttcagg caaagcttta atttatacta agcttaggaa 4620
gtatggctaa tgccaacggc agtttttttc ttcttaattc cacatgactg aggcatatat 4680
gatctctggg taggtgagtt gttgtgacaa ccacaagcac tttttttttt tttaaagaaa 4740
aaaaggtagt gaatttttaa tcatctggac tttaagaagg attctggagt atacttaggc 4800
ctgaaattat atatatttgg cttggaaatg tgtttttctt caattacatc tacaagtaag 4860
tacagctgaa attcagagga cccataagag ttcacatgaa aaaaatcaat ttatttgaaa 4920
aggcaagatg caggagagag gaagccttgc aaacctgcag actgcttttt gcccaatata 4980
gattgggtaa ggctgcaaaa cataagctta attagctcac atgctctgct ctcacgtggc 5040
accagtggat agtgtgagag aattaggctg tagaacaaat ggccttctct ttcagcattc 5100
acaccactac aaaatcatct tttatatcaa cagaagaata agcataaact aagcaaaagg 5160
tcaataagta cctgaaacca agattggcta gagatatatc ttaatgcaat ccattttctg 5220
atggattgtt acgagttggc tatataatgt atgtatggta ttttgatttg tgtaaaagtt 5280
ttaaaaatca agctttaagt acatggacat ttttaaataa aatatttaaa gacaatttag 5340
aaaattgcct taatatcatt gttggctaaa tagaataggg gacatgcata ttaaggaaaa 5400
ggtcatggag aaataatatt ggtatcaaac aaatacattg atttgtcatg atacacattg 5460
aatttgatcc aatagtttaa ggaataggta ggaaaatttg gtttctattt ttcgatttcc 5520
tgtaaatcag tgacataaat aattcttagc ttattttata tttccttgtc ttaaatactg 5580
agctcagtaa gttgtgttag gggattattt ctcagttgag actttcttat atgacatttt 5640
actatgtttt gacttcctga ctattaaaaa taaatagtag atacaatttt cataaagtga 5700
agaattatat aatcactgct ttataactga ctttattata tttatttcaa agttcattta 5760
aaggctacta ttcatcctct gtgatggaat ggtcaggaat ttgttttctc atagtttaat 5820
tccaacaaca atattagtcg tatccaaaat aacctttaat gctaaacttt actgatgtat 5880
atccaaagct tctcattttc agacagatta atccagaagc agtcataaac agaagaatag 5940
gtggtatgtt cctaatgata ttatttctac taatggaata aactgtaata ttagaaatta 6000
tgctgctaat tatatcagct ctgaggtaat ttctgaaatg ttcagactca gtcggaacaa 6060
attggaaaat ttaaattttt attcttagct ataaagcaag aaagtaaaca cattaatttc 6120
ctcaacattt ttaagccaat taaaaatata aaagatacac accaatatct tcttcaggct 6180
ctgacaggcc tcctggaaac ttccacatat ttttcaactg cagtataaag tcagaaaata 6240
aagttaacat aactttcact aacacacaca tatgtagatt tcacaaaatc cacctataat 6300
tggtcaaagt ggttgagaat atatttttta gtaattgcat gcaaaatttt tctagcttcc 6360
atcctttctc cctcgtttct tctttttttg ggggagctgg taactgatga aatcttttcc 6420
caccttttct cttcaggaaa tataagtggt tttgtttggt taacgtgata cattctgtat 6480
gaatgaaaca ttggagggaa acatctactg aatttctgta atttaaaata ttttgctgct 6540
agttaactat gaacagatag aagaatctta cagatgctgc tataaataag tagaaaatat 6600
aaatttcatc actaaaatat gctattttaa aatctatttc ctatattgta tttctaatca 6660
gatgtattac tcttattatt tctattgtat gtgttaatga ttttatgtaa aaatgtaatt 6720
gcttttcatg agtagtatga ataaaattga ttagtttgtg ttttcttgtc tccc 6774
<210> SEQ ID NO 55
<211> LENGTH: 1548
<212> TYPE: DNA
<213> ORGANISM: Homo sapiens
<400> SEQUENCE: 55
gacctttcag agccaggagg gctttcgggg gcgtggggcg cgctgcggag cggagccgcg 60
gctcgacggc ggtgcgctgg cggcgagtgt atgcagacgg cgcccggccc gaaccccgag 120
ccccgcgggg ctccccaccc gccggcctcc cgcccctccc gcgcctccgc ctggggacca 180
cgtcggcctt ttgttggcga accgtccttt ctttcagcgc tttgcgcagc aacggaaatt 240
tcattgctcc tgggtggaaa ttaaagggac tcgcgttccc tctctccctc tccctctccc 300
actctccctc tctttctctc tctcgcccac ccttccccct tcttccccca cctttcccgc 360
gaagccggag tcagcatctc caggcgcggg atcccgctcc gagcacctcg cagctgtccg 420
gctgccgccc cttccatggg cgccgcgctc gcctgcagcc gccgccgccg cggggcgggc 480
gcgatgccac gatgggccta atctggctgc tactgctcag cctgctggag cccggctggc 540
ccgcagcggg ccctggggcg cggttgcggc gcgatgcggg cggccgtggc ggcgtctacg 600
agcaccttgg cggggcgccc cggcgccgca agctctactg cgccacgaag taccacctcc 660
agctgcaccc gagcggccgc gtcaacggca gcctggagaa cagcgcctac agtattttgg 720
agataacggc agtggaggtg ggcattgtgg ccatcagggg tctcttctcc gggcggtacc 780
tggccatgaa caagagggga cgactctatg cttcggagca ctacagcgcc gagtgcgagt 840
ttgtggagcg gatccacgag ctgggctata atacgtatgc ctcccggctg taccggacgg 900
tgtctagtac gcctggggcc cgccggcagc ccagcgccga gagactgtgg tacgtgtctg 960
tgaacggcaa gggccggccc cgcaggggct tcaagacccg ccgcacacag aagtcctccc 1020
tgttcctgcc ccgcgtgctg gaccacaggg accacgagat ggtgcggcag ctacagagtg 1080
ggctgcccag accccctggt aagggggtcc agccccgacg gcggcggcag aagcagagcc 1140
cggataacct ggagccctct cacgttcagg cttcgagact gggctcccag ctggaggcca 1200
gtgcgcacta gctgggcctg gtggccaccg ccagagctcc tggcgacatc ttggcgtggc 1260
agcctcttga ctctgactct cctccttgag cccttgcccc tgcgtcccgc gtctgggttc 1320
tcagctattt ccagagccag ctcaaatcag ggtccagtgg gaactgaaga gggcccaagt 1380
cggagctcgg agggggctgc ctgcaatgca gggcatttgt gggtctgtgt ggcaggaagc 1440
cggcagggaa gggcctgagt gccagccctg gcagactgag gagcctccca ggagcagcgg 1500
ggcagtgtgg ggctttgtgt catcacaaca ttaaagtatt ttattcta 1548
<210> SEQ ID NO 56
<211> LENGTH: 1220
<212> TYPE: DNA
<213> ORGANISM: Homo sapiens
<400> SEQUENCE: 56
gggagcgggc gagtaggagg gggcgccggg ctatatatat agcggctcgg cctcgggcgg 60
gcctggcgct cagggaggcg cgcactgctc ctcagagtcc cagctccagc cgcgcgcttt 120
ccgcccggct cgccgctcca tgcagccggg gtagagcccg gcgcccgggg gccccgtcgc 180
ttgcctcccg cacctcctcg gttgcgcact cctgcccgag gtcggccgtg cgctcccgcg 240
ggacgccaca ggcgcagctc tgccccccag cttcccgggc gcactgaccg cctgaccgac 300
gcacggccct cgggccggga tgtcggggcc cgggacggcc gcggtagcgc tgctcccggc 360
ggtcctgctg gccttgctgg cgccctgggc gggccgaggg ggcgccgccg cacccactgc 420
acccaacggc acgctggagg ccgagctgga gcgccgctgg gagagcctgg tggcgctctc 480
gttggcgcgc ctgccggtgg cagcgcagcc caaggaggcg gccgtccaga gcggcgccgg 540
cgactacctg ctgggcatca agcggctgcg gcggctctac tgcaacgtgg gcatcggctt 600
ccacctccag gcgctccccg acggccgcat cggcggcgcg cacgcggaca cccgcgacag 660
cctgctggag ctctcgcccg tggagcgggg cgtggtgagc atcttcggcg tggccagccg 720
gttcttcgtg gccatgagca gcaagggcaa gctctatggc tcgcccttct tcaccgatga 780
gtgcacgttc aaggagattc tccttcccaa caactacaac gcctacgagt cctacaagta 840
ccccggcatg ttcatcgccc tgagcaagaa tgggaagacc aagaagggga accgagtgtc 900
gcccaccatg aaggtcaccc acttcctccc caggctgtga ccctccagag gacccttgcc 960
tcagcctcgg gaagcccctg ggagggcagt gccgagggtc accttggtgc actttcttcg 1020
gatgaagagt ttaatgcaag agtaggtgta agatatttaa attaattatt taaatgtgta 1080
tatattgcca ccaaattatt tatagttctg cgggtgtgtt ttttaatttt ctggggggaa 1140
aaaaagacaa aacaaaaaac caactctgac ttttctggtg caacagtgga gaatcttacc 1200
attggatttc tttaacttgt 1220
<210> SEQ ID NO 57
<211> LENGTH: 5399
<212> TYPE: DNA
<213> ORGANISM: Homo sapiens
<400> SEQUENCE: 57
ggggaagctt cgcaggcgtg cacggagcag tgagatcact ggcgttataa atatcccggt 60
gccagcgcgg agatccgctc gggtggcctc tctcttcccc tctccccttc tcttccccga 120
ggctatgtcc acccggtgcg gcgaggcggg cagagccaga ggcacgcagc cgcacagggg 180
ctacagagcc cagaatcagc cctacaagat gcacttagga cccccgcggc tggaagaatg 240
agcttgtcct tcctcctcct cctcttcttc agccacctga tcctcagcgc ctgggctcac 300
ggggagaagc gtctcgcccc caaagggcaa cccggacccg ctgccactga taggaaccct 360
agaggctcca gcagcagaca gagcagcagt agcgctatgt cttcctcttc tgcctcctcc 420
tcccccgcag cttctctggg cagccaagga agtggcttgg agcagagcag tttccagtgg 480
agcccctcgg ggcgccggac cggcagcctc tactgcagag tgggcatcgg tttccatctg 540
cagatctacc cggatggcaa agtcaatgga tcccacgaag ccaatatgtt aagtgttttg 600
gaaatatttg ctgtgtctca ggggattgta ggaatacgag gagttttcag caacaaattt 660
ttagcgatgt caaaaaaagg aaaactccat gcaagtgcca agttcacaga tgactgcaag 720
ttcagggagc gttttcaaga aaatagctat aatacctatg cctcagcaat acatagaact 780
gaaaaaacag ggcgggagtg gtatgtggcc ctgaataaaa gaggaaaagc caaacgaggg 840
tgcagccccc gggttaaacc ccagcatatc tctacccatt ttctgccaag attcaagcag 900
tcggagcagc cagaactttc tttcacggtt actgttcctg aaaagaaaaa gccacctagc 960
cctatcaagc caaagattcc cctttctgca cctcggaaaa ataccaactc agtgaaatac 1020
agactcaagt ttcgctttgg ataatattcc tcttggcctt gtgagaaacc attctttccc 1080
ctcaggagtt tctataggtg tcttcagagt tctgaagaaa aattactgga cacagcttca 1140
gctatactta cactgtattg aagtcacgtc atttgtttca atgtgactga aacaaaatgt 1200
tttttgatag gaaggaaact ggaattcttt gtactaatac agggagcaca ctccttcagt 1260
tcagcaagac ataaagcctt ttgctttatg cttgagggat atttagaact ttgtattttc 1320
ggaaagttaa ataacaggga ctacgtattt ttctgacttt tacagattaa cctgaaagaa 1380
catacatgat acatttttat ttttggtttc caaagaatat tttgatgcag ataaaatatt 1440
ttgttaactt ttgttttttt ttgtttgttt tcttaaaagt acctctgcat tgagcatatt 1500
ttcttacttt tattatttta attaatatga cataagcaat cattttatgc tgtttatgaa 1560
ttataaatgt gtttatagct catttgtaat atggaaatct tttacatttt tcctattcac 1620
tgcacttttt tattgttttt atttctagcc atacctcaga taatatgttt agttttacat 1680
tttaaaatgt ttaaattctc tttcacagca ccaaaggctc agcttggatt tgtgtgtatg 1740
tgtatgtcaa ttcatgacat tatgtggaat cctaaacctt tggtggctgg gatatgatgg 1800
gttagaagca aggagaaaat ataaggactt tttgatggaa ttaaatgtgg gaggtaagga 1860
aaaggattta gaggtaaaag tacactaagt ttgcaacatt tattgagatc taagtctgtc 1920
ttgccttcat ttctcttttt atctccccct tgccctcatt cttgaacagc tggaggaata 1980
cattttattc tgtccatgaa gcatacacta tgaaattcaa gtgcttaaaa atacttctat 2040
gactctctgc tatcccactg tatagatcca cagggagcaa acacttagaa atgatagaga 2100
actgaaggag atcaatggtt taacagttat ccatgccaag tcccattgtc agaaatattc 2160
ttattactca gtcaaacact ctttgagctt cccttcctaa aggtaaccaa tccagtgaat 2220
agatgtgccc ttttataagg aaacttctga tgtttattaa aaaaactggc cttttgatag 2280
aggtaactta atttgggaat ttgttgtgtt gaaatggcat ttaatttcaa cctaaatact 2340
gactgctgga cataaatcac agaaaattta acttaagaaa atttacaaaa tttattctca 2400
ggtaatcatt ttaataaagt tctgcaaaat acacgtttat cttacattca gaaatgtggc 2460
aaaaaaggca tagctaaagg ctaaacatat ggctttagta gtaacaaaag ggttcataga 2520
aacttcatgg tttgcattta aacatgttta aagtgtactt ataaactatt tttttcttaa 2580
agcaaactat gatttatttt ggtgcacaaa tacaaagtgg aaacttacca aaattgaact 2640
agctaccata taagcagatt gctttaattt gatgggaaaa tagtacacac atatatataa 2700
caaataatat attaaaaaac ccatccatca actaaaacat tatatgtata catcagtata 2760
gtgttttatt ataaagccaa ttatctgatt aagcattctt tccactgaat gcataatgtt 2820
taaatagcat aaaatgaaat gctacaaaaa ttgaactaat ttatacttta aagtatttct 2880
gggttaaatg aaacaatgaa attttttagt atgttcaact ctcatccaaa tggcatatga 2940
ccctgtttac acagcctaaa gctaaaaata ttactctagt ttattctaat ctattgttaa 3000
gtattgtgca ctgtatacca agttcttagg gcacatgaaa aattttagct gccaaacagg 3060
aactagtaaa catatgttcc taataagtga agggaaagat aataatgatg gtcaacaata 3120
agccacgtca atgcataagt tgtataggct aaatgttgct tgtaggctac attaaactca 3180
aatgtaatag tttatcttat actcctggtt tgatttgatt agcatattaa cgtgaaagta 3240
ggatagctac taaatatata ttatgcaagt caggaatcat taatttcaaa atttaaagcc 3300
atgctaaaat taaaaagaaa atattaaatt acacaattac acttgtcttt actggccata 3360
caaaatgatt tttttttttt ttttgagaca gagtcttgct ctgtcaccag gctggagtgc 3420
agtggcatga tctcggctca ctgcaacctc caactccctg gtttaaggga ttctcctgcc 3480
tcagcctccc aagtagctgg gattacagac tcatgccacc acgccagcta atttttgtat 3540
ttttagtaga gacggggttt caccatgttg gtcaggatgg tctcaatcct ggcctcttga 3600
tagtcctgac ctcatgatct gcccacctcg gcctccccaa agtgctggga ttacaggtac 3660
aatgatgtat aattaatgct tagtgaagca taaagttacc tacatcaatt aattaaatga 3720
acttatgtac agaaaacatg tataaatata agtctatact aatgcttaca actttctaag 3780
agggttcttg cttatgtagc tttttattat tttaagtaac tagaaccacc aaatatcaaa 3840
taaaattatt tggttatggt tatgttcatc taaacacaac aataactttt atattaatat 3900
ttaggagtct attttgtcta taggtgacaa acatctccag actaacatgt cagttttatc 3960
aattatatta tgtttaatta tttaagattt ctttatgtgg aacatctata gagataaata 4020
gaaattttca ataagatgta gtaacactgt gatttatctt tcaagagtct ctcttcactt 4080
ccttctaaag agactaattt gagagtacag gtgcatatta attttcttgg ttctttcagc 4140
tgaattatat tggtccagaa gttcaaaatc atgtgacaat aataagggat actgacagaa 4200
gttatttcca agtttgtgta tatattataa aaattacata tataaaacta aggcttttat 4260
ttctgttatt tttaagcttt tatttcttgt agctaaaaat aaaacatcat aaatctggta 4320
ggtaaatttc ttattaaatc aatcttgaaa tagaaaatgt aataactttc ttaccattaa 4380
cattttttac ccttccatag aagggaggga ataaatcatg acttatccca ttttcaataa 4440
caaaacgaaa ctatggcact aaccaaaaac ttgcattctg gcataatttt tacagttgca 4500
gagaattgtt tctgggctca ttaaaaaaag tagtattgca gacattgctg caatgggaag 4560
cagacaataa cttcttaaag gaattctaca cctcctttaa gatttactta attgctacat 4620
ctaaattctg ataatttaaa atccatttta ggtgataaaa ttttttaaaa gttttgaagg 4680
aaacctctgg ataaatggac aaggcctaat ttttttttgt agtcaatcca actgtactgg 4740
ccaatttttg aaataagatt atatgattag gtattagcag agacaaagag ttacctcctc 4800
catcttactc tgccctattt gaaagtctca ggggagaaaa gggaacaaga tgctgatcca 4860
acctgagtgg agtcaggtga ggcatcttta catctaagaa ttttttttta aattttatta 4920
ttattatact tcaagttcta gggtacatgt ccacaatgca catgtctgtc acacatgcac 4980
acatgtgcca tgctggtgtg ctgcacccac caacctgtca tccagcatta ggtatatctc 5040
ctaatgctat ccctcccctc tccacccacc ccacagcagg ccccggtatg tgatgttccc 5100
cttcgtgtgt ccatgtgttc ttattgttca attcccacct atgagtgaga atatgtggtg 5160
tttggttttt ggtccttgca atagtttgct gagaatgatg gtttccagct tcatccatgt 5220
ccctacaaag aacatgaact catcattttt tatggctgca tagtattcca tggtgtatat 5280
gtgccacatt ttcttaatcc agtctatcat tgttggacat ttgggttggt tccaagtctt 5340
tgctattgtg aatagtgctg caataaacat atgtgtgcat gtgtctttaa aaaaaaaaa 5399
<210> SEQ ID NO 58
<211> LENGTH: 5295
<212> TYPE: DNA
<213> ORGANISM: Homo sapiens
<400> SEQUENCE: 58
ggggaagctt cgcaggcgtg cacggagcag tgagatcact ggcgttataa atatcccggt 60
gccagcgcgg agatccgctc gggtggcctc tctcttcccc tctccccttc tcttccccga 120
ggctatgtcc acccggtgcg gcgaggcggg cagagccaga ggcacgcagc cgcacagggg 180
ctacagagcc cagaatcagc cctacaagat gcacttagga cccccgcggc tggaagaatg 240
agcttgtcct tcctcctcct cctcttcttc agccacctga tcctcagcgc ctgggctcac 300
ggggagaagc gtctcgcccc caaagggcaa cccggacccg ctgccactga taggaaccct 360
agaggctcca gcagcagaca gagcagcagt agcgctatgt cttcctcttc tgcctcctcc 420
tcccccgcag cttctctggg cagccaagga agtggcttgg agcagagcag tttccagtgg 480
agcccctcgg ggcgccggac cggcagcctc tactgcagag tgggcatcgg tttccatctg 540
cagatctacc cggatggcaa agtcaatgga tcccacgaag ccaatatgtt aagccaagtt 600
cacagatgac tgcaagttca gggagcgttt tcaagaaaat agctataata cctatgcctc 660
agcaatacat agaactgaaa aaacagggcg ggagtggtat gtggccctga ataaaagagg 720
aaaagccaaa cgagggtgca gcccccgggt taaaccccag catatctcta cccattttct 780
gccaagattc aagcagtcgg agcagccaga actttctttc acggttactg ttcctgaaaa 840
gaaaaagcca cctagcccta tcaagccaaa gattcccctt tctgcacctc ggaaaaatac 900
caactcagtg aaatacagac tcaagtttcg ctttggataa tattcctctt ggccttgtga 960
gaaaccattc tttcccctca ggagtttcta taggtgtctt cagagttctg aagaaaaatt 1020
actggacaca gcttcagcta tacttacact gtattgaagt cacgtcattt gtttcaatgt 1080
gactgaaaca aaatgttttt tgataggaag gaaactggaa ttctttgtac taatacaggg 1140
agcacactcc ttcagttcag caagacataa agccttttgc tttatgcttg agggatattt 1200
agaactttgt attttcggaa agttaaataa cagggactac gtatttttct gacttttaca 1260
gattaacctg aaagaacata catgatacat ttttattttt ggtttccaaa gaatattttg 1320
atgcagataa aatattttgt taacttttgt ttttttttgt ttgttttctt aaaagtacct 1380
ctgcattgag catattttct tacttttatt attttaatta atatgacata agcaatcatt 1440
ttatgctgtt tatgaattat aaatgtgttt atagctcatt tgtaatatgg aaatctttta 1500
catttttcct attcactgca cttttttatt gtttttattt ctagccatac ctcagataat 1560
atgtttagtt ttacatttta aaatgtttaa attctctttc acagcaccaa aggctcagct 1620
tggatttgtg tgtatgtgta tgtcaattca tgacattatg tggaatccta aacctttggt 1680
ggctgggata tgatgggtta gaagcaagga gaaaatataa ggactttttg atggaattaa 1740
atgtgggagg taaggaaaag gatttagagg taaaagtaca ctaagtttgc aacatttatt 1800
gagatctaag tctgtcttgc cttcatttct ctttttatct cccccttgcc ctcattcttg 1860
aacagctgga ggaatacatt ttattctgtc catgaagcat acactatgaa attcaagtgc 1920
ttaaaaatac ttctatgact ctctgctatc ccactgtata gatccacagg gagcaaacac 1980
ttagaaatga tagagaactg aaggagatca atggtttaac agttatccat gccaagtccc 2040
attgtcagaa atattcttat tactcagtca aacactcttt gagcttccct tcctaaaggt 2100
aaccaatcca gtgaatagat gtgccctttt ataaggaaac ttctgatgtt tattaaaaaa 2160
actggccttt tgatagaggt aacttaattt gggaatttgt tgtgttgaaa tggcatttaa 2220
tttcaaccta aatactgact gctggacata aatcacagaa aatttaactt aagaaaattt 2280
acaaaattta ttctcaggta atcattttaa taaagttctg caaaatacac gtttatctta 2340
cattcagaaa tgtggcaaaa aaggcatagc taaaggctaa acatatggct ttagtagtaa 2400
caaaagggtt catagaaact tcatggtttg catttaaaca tgtttaaagt gtacttataa 2460
actatttttt tcttaaagca aactatgatt tattttggtg cacaaataca aagtggaaac 2520
ttaccaaaat tgaactagct accatataag cagattgctt taatttgatg ggaaaatagt 2580
acacacatat atataacaaa taatatatta aaaaacccat ccatcaacta aaacattata 2640
tgtatacatc agtatagtgt tttattataa agccaattat ctgattaagc attctttcca 2700
ctgaatgcat aatgtttaaa tagcataaaa tgaaatgcta caaaaattga actaatttat 2760
actttaaagt atttctgggt taaatgaaac aatgaaattt tttagtatgt tcaactctca 2820
tccaaatggc atatgaccct gtttacacag cctaaagcta aaaatattac tctagtttat 2880
tctaatctat tgttaagtat tgtgcactgt ataccaagtt cttagggcac atgaaaaatt 2940
ttagctgcca aacaggaact agtaaacata tgttcctaat aagtgaaggg aaagataata 3000
atgatggtca acaataagcc acgtcaatgc ataagttgta taggctaaat gttgcttgta 3060
ggctacatta aactcaaatg taatagttta tcttatactc ctggtttgat ttgattagca 3120
tattaacgtg aaagtaggat agctactaaa tatatattat gcaagtcagg aatcattaat 3180
ttcaaaattt aaagccatgc taaaattaaa aagaaaatat taaattacac aattacactt 3240
gtctttactg gccatacaaa atgatttttt tttttttttt gagacagagt cttgctctgt 3300
caccaggctg gagtgcagtg gcatgatctc ggctcactgc aacctccaac tccctggttt 3360
aagggattct cctgcctcag cctcccaagt agctgggatt acagactcat gccaccacgc 3420
cagctaattt ttgtattttt agtagagacg gggtttcacc atgttggtca ggatggtctc 3480
aatcctggcc tcttgatagt cctgacctca tgatctgccc acctcggcct ccccaaagtg 3540
ctgggattac aggtacaatg atgtataatt aatgcttagt gaagcataaa gttacctaca 3600
tcaattaatt aaatgaactt atgtacagaa aacatgtata aatataagtc tatactaatg 3660
cttacaactt tctaagaggg ttcttgctta tgtagctttt tattatttta agtaactaga 3720
accaccaaat atcaaataaa attatttggt tatggttatg ttcatctaaa cacaacaata 3780
acttttatat taatatttag gagtctattt tgtctatagg tgacaaacat ctccagacta 3840
acatgtcagt tttatcaatt atattatgtt taattattta agatttcttt atgtggaaca 3900
tctatagaga taaatagaaa ttttcaataa gatgtagtaa cactgtgatt tatctttcaa 3960
gagtctctct tcacttcctt ctaaagagac taatttgaga gtacaggtgc atattaattt 4020
tcttggttct ttcagctgaa ttatattggt ccagaagttc aaaatcatgt gacaataata 4080
agggatactg acagaagtta tttccaagtt tgtgtatata ttataaaaat tacatatata 4140
aaactaaggc ttttatttct gttattttta agcttttatt tcttgtagct aaaaataaaa 4200
catcataaat ctggtaggta aatttcttat taaatcaatc ttgaaataga aaatgtaata 4260
actttcttac cattaacatt ttttaccctt ccatagaagg gagggaataa atcatgactt 4320
atcccatttt caataacaaa acgaaactat ggcactaacc aaaaacttgc attctggcat 4380
aatttttaca gttgcagaga attgtttctg ggctcattaa aaaaagtagt attgcagaca 4440
ttgctgcaat gggaagcaga caataacttc ttaaaggaat tctacacctc ctttaagatt 4500
tacttaattg ctacatctaa attctgataa tttaaaatcc attttaggtg ataaaatttt 4560
ttaaaagttt tgaaggaaac ctctggataa atggacaagg cctaattttt ttttgtagtc 4620
aatccaactg tactggccaa tttttgaaat aagattatat gattaggtat tagcagagac 4680
aaagagttac ctcctccatc ttactctgcc ctatttgaaa gtctcagggg agaaaaggga 4740
acaagatgct gatccaacct gagtggagtc aggtgaggca tctttacatc taagaatttt 4800
tttttaaatt ttattattat tatacttcaa gttctagggt acatgtccac aatgcacatg 4860
tctgtcacac atgcacacat gtgccatgct ggtgtgctgc acccaccaac ctgtcatcca 4920
gcattaggta tatctcctaa tgctatccct cccctctcca cccaccccac agcaggcccc 4980
ggtatgtgat gttccccttc gtgtgtccat gtgttcttat tgttcaattc ccacctatga 5040
gtgagaatat gtggtgtttg gtttttggtc cttgcaatag tttgctgaga atgatggttt 5100
ccagcttcat ccatgtccct acaaagaaca tgaactcatc attttttatg gctgcatagt 5160
attccatggt gtatatgtgc cacattttct taatccagtc tatcattgtt ggacatttgg 5220
gttggttcca agtctttgct attgtgaata gtgctgcaat aaacatatgt gtgcatgtgt 5280
ctttaaaaaa aaaaa 5295
<210> SEQ ID NO 59
<211> LENGTH: 744
<212> TYPE: DNA
<213> ORGANISM: Homo sapiens
<400> SEQUENCE: 59
tttagggcca ttaattctga ccacgtgcct gagaggcaag gtggatggcc ctgggacaga 60
aactgttcat cactatgtcc cggggagcag gacgtctgca gggcacgctg tgggctctcg 120
tcttcctagg catcctagtg ggcatggtgg tgccctcgcc tgcaggcacc cgtgccaaca 180
acacgctgct ggactcgagg ggctggggca ccctgctgtc caggtctcgc gcggggctag 240
ctggagagat tgccggggtg aactgggaaa gtggctattt ggtggggatc aagcggcagc 300
ggaggctcta ctgcaacgtg ggcatcggct ttcacctcca ggtgctcccc gacggccgga 360
tcagcgggac ccacgaggag aacccctaca gcctgctgga aatttccact gtggagcgag 420
gcgtggtgag tctctttgga gtgagaagtg ccctcttcgt tgccatgaac agtaaaggaa 480
gattgtacgc aacgcccagc ttccaagaag aatgcaagtt cagagaaacc ctcctgccca 540
acaattacaa tgcctacgag tcagacttgt accaagggac ctacattgcc ctgagcaaat 600
acggacgggt aaagcggggc agcaaggtgt ccccgatcat gactgtcact catttccttc 660
ccaggatcta aggacccaca aaagaaggct tacagattta aagcatcatc tgttcgattg 720
aaattttgca ccagcgaaga attc 744
<210> SEQ ID NO 60
<211> LENGTH: 916
<212> TYPE: DNA
<213> ORGANISM: Homo sapiens
<400> SEQUENCE: 60
acccgcaccc tctccgctcg cgccctgctc agcgcgtcct cccgcggcgg cccgcgggac 60
ggcgtgaccc gccgggctct cggtgccccg gggccgcgcg ccatgggcag cccccgctcc 120
gcgctgagct gcctgctgtt gcacttgctg gtcctctgcc tccaagccca gcatgtgagg 180
gagcagagcc tggtgacgga tcagctcagc cgccgcctca tccggaccta ccaactctac 240
agccgcacca gcgggaagca cgtgcaggtc ctggccaaca agcgcatcaa cgccatggca 300
gaggacggcg accccttcgc aaagctcatc gtggagacgg acacctttgg aagcagagtt 360
cgagtccgag gagccgagac gggcctctac atctgcatga acaagaaggg gaagctgatc 420
gccaagagca acggcaaagg caaggactgc gtcttcacgg agattgtgct ggagaacaac 480
tacacagcgc tgcagaatgc caagtacgag ggctggtaca tggccttcac ccgcaagggc 540
cggccccgca agggctccaa gacgcggcag caccagcgtg aggtccactt catgaagcgg 600
ctgccccggg gccaccacac caccgagcag agcctgcgct tcgagttcct caactacccg 660
cccttcacgc gcagcctgcg cggcagccag aggacttggg cccccgagcc ccgataggtg 720
ctgcctggcc ctccccacaa tgccagaccg cagagaggct catcctgtag ggcacccaaa 780
actcaagcaa gatgagctgt gcgctgctct gcaggctggg gaggtgctgg gggagccctg 840
ggttccggtt gttgatattg tttgctgttg ggtttttgct gttttttttt tttttttttt 900
ttttaaaaca aaagag 916
<210> SEQ ID NO 61
<211> LENGTH: 949
<212> TYPE: DNA
<213> ORGANISM: Homo sapiens
<400> SEQUENCE: 61
acccgcaccc tctccgctcg cgccctgctc agcgcgtcct cccgcggcgg cccgcgggac 60
ggcgtgaccc gccgggctct cggtgccccg gggccgcgcg ccatgggcag cccccgctcc 120
gcgctgagct gcctgctgtt gcacttgctg gtcctctgcc tccaagccca ggtaactgtt 180
cagtcctcac ctaattttac acagcatgtg agggagcaga gcctggtgac ggatcagctc 240
agccgccgcc tcatccggac ctaccaactc tacagccgca ccagcgggaa gcacgtgcag 300
gtcctggcca acaagcgcat caacgccatg gcagaggacg gcgacccctt cgcaaagctc 360
atcgtggaga cggacacctt tggaagcaga gttcgagtcc gaggagccga gacgggcctc 420
tacatctgca tgaacaagaa ggggaagctg atcgccaaga gcaacggcaa aggcaaggac 480
tgcgtcttca cggagattgt gctggagaac aactacacag cgctgcagaa tgccaagtac 540
gagggctggt acatggcctt cacccgcaag ggccggcccc gcaagggctc caagacgcgg 600
cagcaccagc gtgaggtcca cttcatgaag cggctgcccc ggggccacca caccaccgag 660
cagagcctgc gcttcgagtt cctcaactac ccgcccttca cgcgcagcct gcgcggcagc 720
cagaggactt gggcccccga gccccgatag gtgctgcctg gccctcccca caatgccaga 780
ccgcagagag gctcatcctg tagggcaccc aaaactcaag caagatgagc tgtgcgctgc 840
tctgcaggct ggggaggtgc tgggggagcc ctgggttccg gttgttgata ttgtttgctg 900
ttgggttttt gctgtttttt tttttttttt tttttttaaa acaaaagag 949
<210> SEQ ID NO 62
<211> LENGTH: 1003
<212> TYPE: DNA
<213> ORGANISM: Homo sapiens
<400> SEQUENCE: 62
acccgcaccc tctccgctcg cgccctgctc agcgcgtcct cccgcggcgg cccgcgggac 60
ggcgtgaccc gccgggctct cggtgccccg gggccgcgcg ccatgggcag cccccgctcc 120
gcgctgagct gcctgctgtt gcacttgctg gtcctctgcc tccaagccca ggaaggcccg 180
ggcaggggcc ctgcgctggg cagggagctc gcttccctgt tccgggctgg ccgggagccc 240
cagggtgtct cccaacagca tgtgagggag cagagcctgg tgacggatca gctcagccgc 300
cgcctcatcc ggacctacca actctacagc cgcaccagcg ggaagcacgt gcaggtcctg 360
gccaacaagc gcatcaacgc catggcagag gacggcgacc ccttcgcaaa gctcatcgtg 420
gagacggaca cctttggaag cagagttcga gtccgaggag ccgagacggg cctctacatc 480
tgcatgaaca agaaggggaa gctgatcgcc aagagcaacg gcaaaggcaa ggactgcgtc 540
ttcacggaga ttgtgctgga gaacaactac acagcgctgc agaatgccaa gtacgagggc 600
tggtacatgg ccttcacccg caagggccgg ccccgcaagg gctccaagac gcggcagcac 660
cagcgtgagg tccacttcat gaagcggctg ccccggggcc accacaccac cgagcagagc 720
ctgcgcttcg agttcctcaa ctacccgccc ttcacgcgca gcctgcgcgg cagccagagg 780
acttgggccc ccgagccccg ataggtgctg cctggccctc cccacaatgc cagaccgcag 840
agaggctcat cctgtagggc acccaaaact caagcaagat gagctgtgcg ctgctctgca 900
ggctggggag gtgctggggg agccctgggt tccggttgtt gatattgttt gctgttgggt 960
ttttgctgtt tttttttttt tttttttttt taaaacaaaa gag 1003
<210> SEQ ID NO 63
<211> LENGTH: 1036
<212> TYPE: DNA
<213> ORGANISM: Homo sapiens
<400> SEQUENCE: 63
acccgcaccc tctccgctcg cgccctgctc agcgcgtcct cccgcggcgg cccgcgggac 60
ggcgtgaccc gccgggctct cggtgccccg gggccgcgcg ccatgggcag cccccgctcc 120
gcgctgagct gcctgctgtt gcacttgctg gtcctctgcc tccaagccca ggaaggcccg 180
ggcaggggcc ctgcgctggg cagggagctc gcttccctgt tccgggctgg ccgggagccc 240
cagggtgtct cccaacaggt aactgttcag tcctcaccta attttacaca gcatgtgagg 300
gagcagagcc tggtgacgga tcagctcagc cgccgcctca tccggaccta ccaactctac 360
agccgcacca gcgggaagca cgtgcaggtc ctggccaaca agcgcatcaa cgccatggca 420
gaggacggcg accccttcgc aaagctcatc gtggagacgg acacctttgg aagcagagtt 480
cgagtccgag gagccgagac gggcctctac atctgcatga acaagaaggg gaagctgatc 540
gccaagagca acggcaaagg caaggactgc gtcttcacgg agattgtgct ggagaacaac 600
tacacagcgc tgcagaatgc caagtacgag ggctggtaca tggccttcac ccgcaagggc 660
cggccccgca agggctccaa gacgcggcag caccagcgtg aggtccactt catgaagcgg 720
ctgccccggg gccaccacac caccgagcag agcctgcgct tcgagttcct caactacccg 780
cccttcacgc gcagcctgcg cggcagccag aggacttggg cccccgagcc ccgataggtg 840
ctgcctggcc ctccccacaa tgccagaccg cagagaggct catcctgtag ggcacccaaa 900
actcaagcaa gatgagctgt gcgctgctct gcaggctggg gaggtgctgg gggagccctg 960
ggttccggtt gttgatattg tttgctgttg ggtttttgct gttttttttt tttttttttt 1020
ttttaaaaca aaagag 1036
<210> SEQ ID NO 64
<211> LENGTH: 856
<212> TYPE: DNA
<213> ORGANISM: Homo sapiens
<400> SEQUENCE: 64
accttgcgtc cgcagtaccg acccgcacgc tcttcagcgc atccctagtg aaggaggttc 60
tcccccagcc cgtggctgtt gcacttgctg gtcctctgcc tccaagccca gcatgtgagg 120
gagcagagcc tggtgacgga tcagctcagc cgccgcctca tccggaccta ccaactctac 180
agccgcacca gcgggaagca cgtgcaggtc ctggccaaca agcgcatcaa cgccatggca 240
gaggacggcg accccttcgc aaagctcatc gtggagacgg acacctttgg aagcagagtt 300
cgagtccgag gagccgagac gggcctctac atctgcatga acaagaaggg gaagctgatc 360
gccaagagca acggcaaagg caaggactgc gtcttcacgg agattgtgct ggagaacaac 420
tacacagcgc tgcagaatgc caagtacgag ggctggtaca tggccttcac ccgcaagggc 480
cggccccgca agggctccaa gacgcggcag caccagcgtg aggtccactt catgaagcgg 540
ctgccccggg gccaccacac caccgagcag agcctgcgct tcgagttcct caactacccg 600
cccttcacgc gcagcctgcg cggcagccag aggacttggg cccccgagcc ccgataggtg 660
ctgcctggcc ctccccacaa tgccagaccg cagagaggct catcctgtag ggcacccaaa 720
actcaagcaa gatgagctgt gcgctgctct gcaggctggg gaggtgctgg gggagccctg 780
ggttccggtt gttgatattg tttgctgttg ggtttttgct gttttttttt tttttttttt 840
ttttaaaaca aaagag 856
<210> SEQ ID NO 65
<211> LENGTH: 4545
<212> TYPE: DNA
<213> ORGANISM: Homo sapiens
<400> SEQUENCE: 65
actctgcgcg ccggcggggg ctgcgcagga ggagcgctcc gcccggctac aacgctccgc 60
gagccggcgc ggcaacacct gttcgcggca gcctgggcgg cacgcgagct cccggacgcg 120
gctctcctcg ctcgccgctc gccacccgtt ctaagccaat ggacatctgc cgagcctctg 180
gagaatcctg gatactagct ttggacgcct aaagtttctt cttctttttg ttttattatt 240
attatcattt tttggagggg ggaccgggag gggagatttg tcgccgccac caacgtgaga 300
tttttttttc cccttgaagg attcatgctg atgtctgcag agtcggttag agagtaaaaa 360
cagcgcatgc cttcctggag tcaggatccg taaattctga cgtagcccgt gcatcttaaa 420
aatccctata ataacgccta ggcatttaag ttgctatggt cattctgatc tcaaaccaaa 480
tggagaaact acggattttt tttccttatt acggtcggat gggatgaaga ccttcctgcc 540
tgctaagagc tggggatcta tctatagaga tacatagata tgtttatcaa tatgtcagtg 600
tgtgagtata aagtggtggt ttcttagact atcagtggtt tgaccttgaa cctgtgccag 660
tgaaacagca gattactttt atttatgcat ttaatggatt gaagaaaaga accttttttt 720
tctctctctc tctgcaactg cagtaaggga ggggagttgg atatacctcg cctaatatct 780
cctgggttga caccatcatt attgtttatt cttgtgctcc aaaagccgag tcctctgatg 840
gctcccttag gtgaagttgg gaactatttc ggtgtgcagg atgcggtacc gtttgggaat 900
gtgcccgtgt tgccggtgga cagcccggtt ttgttaagtg accacctggg tcagtccgaa 960
gcaggggggc tccccagggg acccgcagtc acggacttgg atcatttaaa ggggattctc 1020
aggcggaggc agctatactg caggactgga tttcacttag aaatcttccc caatggtact 1080
atccagggaa ccaggaaaga ccacagccga tttggcattc tggaatttat cagtatagca 1140
gtgggcctgg tcagcattcg aggcgtggac agtggactct acctcgggat gaatgagaag 1200
ggggagctgt atggatcaga aaaactaacc caagagtgtg tattcagaga acagttcgaa 1260
gaaaactggt ataatacgta ctcatcaaac ctatataagc acgtggacac tggaaggcga 1320
tactatgttg cattaaataa agatgggacc ccgagagaag ggactaggac taaacggcac 1380
cagaaattca cacatttttt acctagacca gtggaccccg acaaagtacc tgaactgtat 1440
aaggatattc taagccaaag ttgacaaaga cagtttcttc acttgagccc ttaaaaaagt 1500
aaccactata aaggtttcac gcggtgggtt cttattgatt cgctgtgtca tcacatcagc 1560
tccactgttg ccaaactttg tcgcatgcat aatgtatgat ggaggcttgg atgggaatat 1620
gctgattttg ttctgcactt aaaggcttct cctcctggag ggctgcctag ggccacttgc 1680
ttgatttatc atgagagaag aggagagaga gagagactga gcgctaggag tgtgtgtatg 1740
tgtgtgtgtg tgtgtgtgtg tgtgtgtgta tgtgtgtagc gggagatgtg ggcggagcga 1800
gagcaaaagg actgcggcct gatgcatgct ggaaaaagac acgcttttca tttctgatca 1860
gttgtacttc atcctatatc agcacagctg ccatacttcg acttatcagg attctggctg 1920
gtggcctgcg cgagggtgca gtcttactta aaagactttc agttaattct cactggtatc 1980
atcgcagtga acttaaagca aagacctctt agtaaaaaat aaaaaaaaat aaaaaataaa 2040
aataaaaaaa gttaaattta tttatagaaa ttccaaaggc aacattttat ttattttata 2100
tatttattta ttatatagag tttattttta atgaaacatg tacaggccag ataggcattt 2160
tggaagcttt aggctctgta agcattaaat ggcaaagtcc gctatgaacc tgtggtaaat 2220
tcatgcaagt agatataatg gtgcatggat ataagaaatt ctaatgaccc taatgtacta 2280
aaggcgacaa tctcttttgt gcccatatta ttgtaaactt atgcacatcg ctcatgacac 2340
tgagtattca ctcttcagac tgcttgtttc atagcttatc ccagaggatt aaagataaac 2400
tgggtctcaa actttgattc tgtgtctgca atatttcctc tctcataagt gactccacta 2460
ttgtaacttc atggttggaa aatatgaggg ttgatatatg tcttacttgt ttaaatctgt 2520
cgcagaatat accaaagcta aataataact atgctttcat tttagccgat ctccagaatg 2580
acagtattaa catcaaacat tgtattgatt tagaattctc aaaaaaggaa aaaaaagtac 2640
atagcacaga ctattttttt taaagacgta agaatcagat taacaggatc atacttgtaa 2700
actttttttg gttcacttgg ctatcaaata tgaaattata gaagtatcat aggggtcatt 2760
gtaacatctt ttagagaaaa tggctatcag tgtgaactgt cataattacg tggtaatagc 2820
acccttagta aaacttgcaa aatgaaacta ataaatcgtt atcaataatg acaatgaggg 2880
ggaaagtatt atacttgttg actgtgtttt gttttttaaa atggtctcca caagcgctca 2940
atttttttag aggggatatt actatataga atatctttta caaggctttt ataacatttt 3000
atgctgaaaa gcataagaat acgtatttct ttagtagcaa taattttgga acttgccctt 3060
gggcaagcga gactatttct tactatatac taaggagaaa agagccaaat tcttaaagca 3120
atatttaaga aaaaaggaat ttataacaaa ttctcatcta catatgacac tttctagcca 3180
gttgtgttga gaagtgcaaa gtgacggttt aaacatgtgt tgggatttat tgaactaatt 3240
ttaaaattta ctattcaaac tttattttgc tctgatgcac attctctatg aaaaataaaa 3300
gtgtgtcact ggtgagtgac agctgttatg agctagaagc gcatgactta ttgtgacgat 3360
gtcttgcctt tctgtggtcc aagttggagt acatggcaat gccctcctgc tgatgtgcat 3420
taaggaaaat ctaagtctaa tatttggaat taagatatat tttaggggga ggggacagaa 3480
gcaatgtaaa atagttgatt tatgataaag ctcagaatgt cctcttcatt tattttcttg 3540
ttttattttc ctttctaaac agaaactgca tttaattcca aaaagtagta ttcttattta 3600
ttatttaacc ctttgctgct gctaaaatgt gcacatattc aggctttagt ttttccaaaa 3660
ggcatttttt ttttggctga aaaatattaa acatttgacc acagggaaga atcaagtttc 3720
taggatgtca taggtatact atgtagcact gaaaaaattg attttaggtg acagccaaaa 3780
gtagtcttaa agtagcatga gaccttagat aatcgaccta aaagaaagaa aattgtgaaa 3840
aagacaaaaa tcttcatgca ttcctataaa acgctacttt aaggtctact tttggagtta 3900
attttgtttg gtactttttt tttttttaag acgagcaaat tgttatatgc ttttggcaat 3960
tgatacaata aactgtaatg gtctgtaaat aaataaatat tgactcatgc gatttatgta 4020
aatagtggaa ctgggagagt ggatggctca gggtttcggt gtgggcattg tctcttgggc 4080
agtagagtga gtcatcccca gctcatgggt ttgcatccag ttcttgtctt aagagaccca 4140
aagcccagtg aatggcagcc ctgagccact gtggaatggg ggttctggtt tcacaaacag 4200
atgcttagat agccaaacca ctgtcttgtt ggtgccaaca cttgcactgt ggtcaaagac 4260
ttaccgagca tgggctgaac aaccttccca tctgtcatgt gaatgtcccc aagcagtggt 4320
gaaggacatg ctaggtcagt gttggggaac ctgccctgcc aggtcctgtt ttgtagataa 4380
acaaatggct gccttctggt gtttttattc tatttcatct cattaacact acaaccttgt 4440
gttatttact tgataatctg taattgtatg taaatacata caggattatg taatttgtgt 4500
aaatacataa ttacagagtt ttgaaaactg aaaaaaaaaa aaaaa 4545
<210> SEQ ID NO 66
<211> LENGTH: 627
<212> TYPE: DNA
<213> ORGANISM: Homo sapiens
<400> SEQUENCE: 66
atgtggaaat ggatactgac acattgtgcc tcagcctttc cccacctgcc cggctgctgc 60
tgctgctgct ttttgttgct gttcttggtg tcttccgtcc ctgtcacctg ccaagccctt 120
ggtcaggaca tggtgtcacc agaggccacc aactcttctt cctcctcctt ctcctctcct 180
tccagcgcgg gaaggcatgt gcggagctac aatcaccttc aaggagatgt ccgctggaga 240
aagctattct ctttcaccaa gtactttctc aagattgaga agaacgggaa ggtcagcggg 300
accaagaagg agaactgccc gtacagcatc ctggagataa catcagtaga aatcggagtt 360
gttgccgtca aagccattaa cagcaactat tacttagcca tgaacaagaa ggggaaactc 420
tatggctcaa aagaatttaa caatgactgt aagctgaagg agaggataga ggaaaatgga 480
tacaatacct atgcatcatt taactggcag cataatggga ggcaaatgta tgtggcattg 540
aatggaaaag gagctccaag gagaggacag aaaacacgaa ggaaaaacac ctctgctcac 600
tttcttccaa tggtggtaca ctcatag 627
<210> SEQ ID NO 67
<211> LENGTH: 2763
<212> TYPE: DNA
<213> ORGANISM: Homo sapiens
<400> SEQUENCE: 67
gtgggatcca ctgaggagta cataggctgc tggatctggt ggagccagca ctgggcccac 60
gggtggtaac tggctgctgt ggaggggggt acgtgagggg gggggtctgg ggcttatcct 120
caggtcctgt gggtggggca gcgagtcggg gcctgagcgt caagagcatg ccctagtgag 180
cgggctcctc tgggggagcc cagcgcgctc cgggcgcctg ccggtttggg ggtgtctcct 240
cccggggcgc tatggcggcg ctggccagta gcctgatccg gcagaagcgg gaggtccgcg 300
agcccggggg cagccggccg gtgtcggcgc agcggcgcgt gtgtccccgc ggcaccaagt 360
ccctttgcca gaagcagctc ctcatcctgc tgtccaaggt gcgactgtgc ggggggcggc 420
ccgcgcggcc ggaccgcggc ccggagcctc agctcaaagg catcgtcacc aaactgttct 480
gccgccaggg tttctacctc caggcgaatc ccgacggaag catccagggc accccagagg 540
ataccagctc cttcacccac ttcaacctga tccctgtggg cctccgtgtg gtcaccatcc 600
agagcgccaa gctgggtcac tacatggcca tgaatgctga gggactgctc tacagttcgc 660
cgcatttcac agctgagtgt cgctttaagg agtgtgtctt tgagaattac tacgtcctgt 720
acgcctctgc tctctaccgc cagcgtcgtt ctggccgggc ctggtacctc ggcctggaca 780
aggagggcca ggtcatgaag ggaaaccgag ttaagaagac caaggcagct gcccactttc 840
tgcccaagct cctggaggtg gccatgtacc aggagccttc tctccacagt gtccccgagg 900
cctccccttc cagtccccct gccccctgaa atgtagtccc tggactggag gttccctgca 960
ctcccagtga gccagccacc accacaacct gtctcccagt cctgctctca cccctgctgc 1020
cacacacatg ccctgagcag ccaggtccca ctaggtgctc taccctgagg gagcctaggg 1080
gctgactgtg acttccgagg ctgctgagac ccttagatct ttgggcctag gagggagtca 1140
gagaggggga tgtctgaaga tggtcctggc tgatcacttc tttctttcca cactcacaca 1200
accccatgcc ttttcctgag atggcgctgg gagttcccac atggacagcc agggcataaa 1260
cacttcccac cccggctcag ccagttcctg gagtcctgtg ccccttttca ttgccactga 1320
gccatttcta gattcactgg agctcaggat tcatgtgtcc ttctttccct actctacctt 1380
ctaccttggt ctggacacat tctggaacac tggacaccct cgccagggcc acttctgcac 1440
tagggctctg tgctggaacc caggcatgct gccagccttt tctctggatc tgtcaggcct 1500
ctgtccttga ctcagatgga cccctggttt ccaagtagaa agaggctaga tttgggcctt 1560
gtctagctgt tggctttggc ctgaaccgga accagtctca gatgaccacg ggtttaacct 1620
tcttatccca gagacaccca attctagagc tttatggagc cgtacttccc cctgaatcct 1680
agctctagga catagatcat gactctcagc ccttttaccc aggatggagc tggggcctgt 1740
atagccatat tattgttcta agtaagttct agccccaccc tcccgccttc ttgagtgata 1800
cctattacgg atgagttctg gaaaagaccc agctatgatt cataaaaaca cttctggatg 1860
aatcaagaac catttcttgt ttttcctaga taattctcta aaaatatgat tcttccatat 1920
agaatgctaa gcttattttt acatgcagtt tctagctcct tcaacccagc tgaggtcgtg 1980
ccagggagac agagtctgga gaagggcaga ggaattttgg aaggatccct ggctcatagt 2040
agggaagctg ggatggggga ggggtcaaaa ttatggcatg actgaacctg catctgtgtt 2100
gggtggacat gaatacttag ctacctcagc aggaattcct tccaggtccc ctttaaagct 2160
gaggtcctta gagtaatatg tccttaataa aaaggacaaa tggatacagc cttgaccctc 2220
ccagtgagga gaccccaatt cagcaataag tctcaccctt ctcccctaca ggtcaggcca 2280
agaagggtga aggcctcttg cactccagac ctcatacgcc ccaacagctt ctaattggat 2340
agaacttgct ttaccttaca gctcacaacc tcagctgggt tttaggtacc caaaaagggc 2400
ctgtctagat tttttcagaa aaacgtggag tgctaggggc agcctggaaa agatggggaa 2460
cctgctagtg aactaggagg gagacttcca tagcctcaga cttggatagg gtaggctgag 2520
ggggccctaa gggagggact aaggctccaa ggcaggtcac ttttccttag gctgttctac 2580
ttctggcttg ttgcaagagg agtagatgcc ccctcaccca cacaaacccc actcagtctc 2640
cacccaactc ctggcactgc tcccagggga tcgggtctcc actccagctt tctcaattaa 2700
agacgattta tacaaaaaaa aaaaaaaaaa aaaaaaaaaa aaaaaaaaaa aaaaaaaaaa 2760
aaa 2763
<210> SEQ ID NO 68
<211> LENGTH: 6174
<212> TYPE: DNA
<213> ORGANISM: Homo sapiens
<400> SEQUENCE: 68
agtgctgctg gccgggagtt gctctcaccg cagctggaaa cagctgcccc cgccccgcgc 60
ccctacccag actccgggta accgctccca cttcgcgcct ctcggaattc cagaactcgg 120
gtggccggcc cctggaaagc cgcagccggc gcgatgcatt ctgtagacct caccctgctg 180
ggacggacct cctaatcttc agaaccgcgg gccgcaggga gttaaattgc tgccttcctc 240
tccttctctc gtgcggttgg tggcttgttt tctaaaggaa cgttttattc actttttagt 300
attttctacc gggggcgcgc tacccgcctg ggtccagact ctgctttgta aacgggtttt 360
ctatgtatgt atgtgtaggt atactttgga caccttacaa cgcttgcgcc tctccaacag 420
aggcacgtct tgttattttg ggcatcgttc ttccccttcc acttggtacc ccgaacgcag 480
tgtgactaaa ctccccactg ccccttggac gccgatcgcc ttggggtgca agtttggggt 540
gcaaacgtct acttcgcaag agggcctggg accgccccgc cccgcccccc ggccgccaga 600
ggttggggaa gtttacatct ggattttcac acattttgtc gccactgccc agactttgac 660
taaccttgtg agcgccgggt tttcgatact gcagcctcct caaattttag cactgcctcc 720
ccgcgactgc cctttccctg gccgcccagg tcctgccctc gccccggcgg agcgcaagcc 780
ggagggcgca gtagaggctg gggcctgagg ccctcgctga gcagctatgg ctgcggcgat 840
agccagctcc ttgatccggc agaagcggca ggcgagggag tccaacagcg accgagtgtc 900
ggcctccaag cgccgctcca gccccagcaa agacgggcgc tccctgtgcg agaggcacgt 960
cctcggggtg ttcagcaaag tgcgcttctg cagcggccgc aagaggccgg tgaggcggag 1020
accagaaccc cagctcaaag ggattgtgac aaggttattc agccagcagg gatacttcct 1080
gcagatgcac ccagatggta ccattgatgg gaccaaggac gaaaacagcg actacactct 1140
cttcaatcta attcccgtgg gcctgcgtgt agtggccatc caaggagtga aggctagcct 1200
ctatgtggcc atgaatggtg aaggctatct ctacagttca gatgttttca ctccagaatg 1260
caaattcaag gaatctgtgt ttgaaaacta ctatgtgatc tattcttcca cactgtaccg 1320
ccagcaagaa tcaggccgag cttggtttct gggactcaat aaagaaggtc aaattatgaa 1380
ggggaacaga gtgaagaaaa ccaagccctc atcacatttt gtaccgaaac ctattgaagt 1440
gtgtatgtac agagaaccat cgctacatga aattggagaa aaacaagggc gttcaaggaa 1500
aagttctgga acaccaacca tgaatggagg caaagttgtg aatcaagatt caacatagct 1560
gagaactctc cccttcttcc ctctctcatc ccttcccctt cccttccttc ccatttaccc 1620
atttccttcc agtaaatcca cccaaggaga ggaaaataaa atgacaacgc aagacctagt 1680
ggctaagatt ctgcactcaa aatcttcctt tgtgtaggac aagaaaattg aaccaaagct 1740
tgcttgttgc aatgtggtag aaaattcacg tgcacaaaga ttagcacact taaaagcaaa 1800
ggaaaaaata aatcagaact ccataaatat taaattaaac tgtattgtta ttagtagaag 1860
gctaattgta atgaagacat taataaagat gaaataaact tattacttta aaggaaagga 1920
tttggagaat tgaactcaca aactgatgtt atatactcaa tagcttaaac tcatgataat 1980
gctgcgatgt gtggttttgc ttgattttgt attttatttg ggcatctgga attgacacac 2040
cattacattc tgtttgcagg attttttttg taaccatgaa attgaacatt tccaaattat 2100
aaactatgtt aatacctata aaatatatag ccaggaacca tttatcatca agaaaagtgt 2160
aagaaattat ttttgagatg taatttaaga ttgttttatg taaaaggaaa atcttgtatg 2220
gcatcgaata gccttaatga gtttaattct ttcacaaaaa tgatttcaaa ttatcctaga 2280
gtataacatt tttatcaaag atattatttc cggagttctt ctttctttct tttttttttt 2340
tttttagtaa tttagcaaaa acattactgt tctaatgctg aagtgacttt tgccagtgcc 2400
atgtccaggt ggtgaggtat aagttacttg ctcttagcat ttggtctgat ttttttgctt 2460
tgtggacacc tttgagagta tccacaaagc aatgtctcag gtgtggacac ctgagagcat 2520
gttttagaaa gctttgtacc ctgtcttgtg gcaggaaaga aagaacaggg gttttacata 2580
aggaaataag tcctaggaaa ttagtcaacg caaattgcat ttgcgtttgt accttaccac 2640
agtcttatat tgttttttaa actctgccat gaaatttgga gacatgactg tgaaattcct 2700
aacttactat cttacaaagc cagtagctaa tttgttgctc tatgtatgat cctgttacaa 2760
gtccagtttg caattcattt gtttcctaga acacagaagg gtaccagtaa tacactaaat 2820
tttcaaggtg tgtagagaaa taatatggaa ttagcagcta tgactccaac agacaggatt 2880
gtgtgagcag ctgaaaggag caaaaaagaa ctcagtgtaa gagaaggcac atacatagtt 2940
aagaatacta aagtattttt aaaaatcaag gaagaaataa atgttacaca atttgcattg 3000
gaataaatag atctatttag tcctacaaat caggagtggt gtagagacat ccaaatttaa 3060
agaaaaaaaa acacaaaaca gaatgttaaa aaatgtatgc agatttatgg atattatcaa 3120
tgagaagaca tagcatgtaa cttctcctat atctctactg tccagcatgt attgttccaa 3180
atatgactcc ctaaaatata tacactttgc agaagctcta ggccctcacc tcaaaccttg 3240
ccattggttg ccgtatttca aggtcaatat agtttccctc actttacaca atcattattc 3300
ttcaatagtg gaccatatcc ttcaccaggt atcctatttc tgttatctag aggttagcag 3360
aaaatgaaat gaaggaattt ccctaagcag ttgggaagaa caaattgtat gcatgtaggc 3420
aaagattttg aagatacatt tgcaagagat atttgtttaa ccaaaatatt tggaaagtaa 3480
caaataaaga catttaaatt ttctaaaaat ggacttgctc ttctaggaaa agaatacccc 3540
tggggcaaaa atataactct agctgtattt cttcttgtca ctcttgattc aacttgatta 3600
taaatacacc tgtcactacc agaaccaaaa aaaaaaagaa aaaaatccca agcacaaagc 3660
ttattttatt tgaaaaaaat aaaaaagaaa cttcaacact atgggacact ggctctttta 3720
gcatgaaatg acttgagctt ttgtagtgat gatacacata cacactcatc agtaaaacga 3780
tggtttcata aataacacaa ttgatgcaaa tcataaaaat caattacaat tatgatttca 3840
tgacaaaata tatttaatta agtttgttat gaaaaaaata gagatatgaa tcactaacaa 3900
aattcctcca ttttcagtgg ctattcatca tttatcatct agactcacat ttgtctcctt 3960
cctgatagca gttaagaaaa aattctaacc acacaatttg tatattgttt ttctccgtat 4020
tatgttaagc aaatgttcac tgcagtaaaa tgttttggaa attagctttg tcttatttcc 4080
agtttagttc agagaattaa ttggaaacct gatttctttt acacataaac ctgacaaaaa 4140
atgtagctta gagcaaaggg tgaatgtttg cttaactcct gcttacttct caagtacatg 4200
aaaactttaa tagaatatgc cagtattcac tgagttttta aaaatattac catgtgtaaa 4260
catataatat ccaacttcat ccaaaaatat ggttgagttt aagtactttg tttttcaggc 4320
ttatttcaag tataataatt ctttgatttt cattgttctg atttctgggt cttcaattca 4380
ttcgtcactt ttccttttta agtaaaataa gctttttttt tttttttttt ttttttttgg 4440
agttgcattg ggatttttcc caggaaaaaa tatggctttt agtaatgctt tgcaattggc 4500
tacgcagata taaattaaga tatgtttatt ctgagttctt attggaataa gtttcaaaat 4560
caacgagctt aagaatgaaa acaaaacttt tgagagtctc acaaaatagc tttctggtca 4620
atacacctta cttgattttt aagctcgcag aataaagtat agaaacaaat ggagctgaag 4680
ttccatttgc taattcagag acttttgtgc ttccgcaaat tggagggcag caagccatcc 4740
tattctcata gtaatcgttt tggctttgaa atttacatac aatttaatag cacattttta 4800
gccattatgg attggcgcaa taaagagata tcaatgtaat gcaatgtgat gctttatggg 4860
cctcattcta attcagaaag cttgtttaaa agaactaaga ctcttctgtt taataaaata 4920
gcaacaatct aatatctaga ttggtagtcc tgcggtgcca ctagtgggag atgagagtat 4980
taagacaaga gtaaggacaa ggaaagactt aaaggttgca tattgaaaag tttggaattc 5040
ctaatttggg agcactgatt tcttggtgaa gaagtaagta tgactacgtt gccagtaatt 5100
ttttaaaaac atagacccag aaatagcaaa tcgatttcac cctcatacct tagtctacaa 5160
ggccttgctc ttgagaaggt tttccatgat attgcttaat ttcatctgca caagatgaga 5220
cacaaacata aaaattccct gctcatttta ataccataaa aggctgaggt tatttctctg 5280
tcataaaatt gtaaatagca ttttttaagt caaaattaca tttaaaacag tggattgttc 5340
tacaaatata tatgtgtata tatacatatg cttctgaaat aaggatatat tatatgagtt 5400
tttatttgat ttgtggtctt tagtcatagg taatcaaaaa taaagagatt tgaatgcaaa 5460
actttataca ttaatgtaca tttctaatga tggtacaaat tgccacttta taataaaaaa 5520
gaaacaggtg ggaataataa tcaaagcacg tgttccttca gtactttggt gatttttaat 5580
cccccttgtg atgcacagga aattattttt tagttacaaa aagttatctt agaaatctat 5640
acttcccaat acagatttca tgttaagtca tatcaaattg agaatttgtg gtgaaagaat 5700
aggaaaagga tgctagatgc tgatctttct ttttcaggat ttttcctgga gcccaagtta 5760
aaaattcaat acttaaatct aagttaagtg aaaattaata atgttcagaa tgatgtattg 5820
agctttagta acagacggaa gcaaaaaaaa ataagaatat ttaacattat gataatagcc 5880
ttaaaataat gtaataaaaa ttgcatcatt aaatgttcta ttagttggaa agaatgagct 5940
gatgtttctt tgtctttgct ccaagtacaa tttaaagaca gtgacattca ttttacttaa 6000
aattgttcaa aaagtccaaa acatactccc atggctagaa ttggtattag ctccaataca 6060
aggttaaatg ttacaatctt aagaaattat tgacactgaa atgtttagta aacatgttgt 6120
atgagaaact aaacaaatta atgtttcatt tttccattaa agcacagatt attc 6174
<210> SEQ ID NO 69
<211> LENGTH: 5408
<212> TYPE: DNA
<213> ORGANISM: Homo sapiens
<400> SEQUENCE: 69
gtgccagcgc ccatgcaaat ctgctgtgca tccagagagc aaagtgggat gatctgtcac 60
tacacctgca gcaccacgct cggaggacag ctcctgcctg cagcttccag acccaggaag 120
cctgagggga aggaaggaag tacgggcgaa atcatcagat tggcttccca gatttgggaa 180
tctgaagcgg gcccacatct tccggccaac ttccattgaa cttcccagca ctcgaaaggg 240
accgaaatgg agagcaaaga accccagctc aaagggattg tgacaaggtt attcagccag 300
cagggatact tcctgcagat gcacccagat ggtaccattg atgggaccaa ggacgaaaac 360
agcgactaca ctctcttcaa tctaattccc gtgggcctgc gtgtagtggc catccaagga 420
gtgaaggcta gcctctatgt ggccatgaat ggtgaaggct atctctacag ttcagatgtt 480
ttcactccag aatgcaaatt caaggaatct gtgtttgaaa actactatgt gatctattct 540
tccacactgt accgccagca agaatcaggc cgagcttggt ttctgggact caataaagaa 600
ggtcaaatta tgaaggggaa cagagtgaag aaaaccaagc cctcatcaca ttttgtaccg 660
aaacctattg aagtgtgtat gtacagagaa ccatcgctac atgaaattgg agaaaaacaa 720
gggcgttcaa ggaaaagttc tggaacacca accatgaatg gaggcaaagt tgtgaatcaa 780
gattcaacat agctgagaac tctccccttc ttccctctct catcccttcc ccttcccttc 840
cttcccattt acccatttcc ttccagtaaa tccacccaag gagaggaaaa taaaatgaca 900
acgcaagacc tagtggctaa gattctgcac tcaaaatctt cctttgtgta ggacaagaaa 960
attgaaccaa agcttgcttg ttgcaatgtg gtagaaaatt cacgtgcaca aagattagca 1020
cacttaaaag caaaggaaaa aataaatcag aactccataa atattaaatt aaactgtatt 1080
gttattagta gaaggctaat tgtaatgaag acattaataa agatgaaata aacttattac 1140
tttaaaggaa aggatttgga gaattgaact cacaaactga tgttatatac tcaatagctt 1200
aaactcatga taatgctgcg atgtgtggtt ttgcttgatt ttgtatttta tttgggcatc 1260
tggaattgac acaccattac attctgtttg caggattttt tttgtaacca tgaaattgaa 1320
catttccaaa ttataaacta tgttaatacc tataaaatat atagccagga accatttatc 1380
atcaagaaaa gtgtaagaaa ttatttttga gatgtaattt aagattgttt tatgtaaaag 1440
gaaaatcttg tatggcatcg aatagcctta atgagtttaa ttctttcaca aaaatgattt 1500
caaattatcc tagagtataa catttttatc aaagatatta tttccggagt tcttctttct 1560
ttcttttttt ttttttttta gtaatttagc aaaaacatta ctgttctaat gctgaagtga 1620
cttttgccag tgccatgtcc aggtggtgag gtataagtta cttgctctta gcatttggtc 1680
tgattttttt gctttgtgga cacctttgag agtatccaca aagcaatgtc tcaggtgtgg 1740
acacctgaga gcatgtttta gaaagctttg taccctgtct tgtggcagga aagaaagaac 1800
aggggtttta cataaggaaa taagtcctag gaaattagtc aacgcaaatt gcatttgcgt 1860
ttgtacctta ccacagtctt atattgtttt ttaaactctg ccatgaaatt tggagacatg 1920
actgtgaaat tcctaactta ctatcttaca aagccagtag ctaatttgtt gctctatgta 1980
tgatcctgtt acaagtccag tttgcaattc atttgtttcc tagaacacag aagggtacca 2040
gtaatacact aaattttcaa ggtgtgtaga gaaataatat ggaattagca gctatgactc 2100
caacagacag gattgtgtga gcagctgaaa ggagcaaaaa agaactcagt gtaagagaag 2160
gcacatacat agttaagaat actaaagtat ttttaaaaat caaggaagaa ataaatgtta 2220
cacaatttgc attggaataa atagatctat ttagtcctac aaatcaggag tggtgtagag 2280
acatccaaat ttaaagaaaa aaaaacacaa aacagaatgt taaaaaatgt atgcagattt 2340
atggatatta tcaatgagaa gacatagcat gtaacttctc ctatatctct actgtccagc 2400
atgtattgtt ccaaatatga ctccctaaaa tatatacact ttgcagaagc tctaggccct 2460
cacctcaaac cttgccattg gttgccgtat ttcaaggtca atatagtttc cctcacttta 2520
cacaatcatt attcttcaat agtggaccat atccttcacc aggtatccta tttctgttat 2580
ctagaggtta gcagaaaatg aaatgaagga atttccctaa gcagttggga agaacaaatt 2640
gtatgcatgt aggcaaagat tttgaagata catttgcaag agatatttgt ttaaccaaaa 2700
tatttggaaa gtaacaaata aagacattta aattttctaa aaatggactt gctcttctag 2760
gaaaagaata cccctggggc aaaaatataa ctctagctgt atttcttctt gtcactcttg 2820
attcaacttg attataaata cacctgtcac taccagaacc aaaaaaaaaa agaaaaaaat 2880
cccaagcaca aagcttattt tatttgaaaa aaataaaaaa gaaacttcaa cactatggga 2940
cactggctct tttagcatga aatgacttga gcttttgtag tgatgataca catacacact 3000
catcagtaaa acgatggttt cataaataac acaattgatg caaatcataa aaatcaatta 3060
caattatgat ttcatgacaa aatatattta attaagtttg ttatgaaaaa aatagagata 3120
tgaatcacta acaaaattcc tccattttca gtggctattc atcatttatc atctagactc 3180
acatttgtct ccttcctgat agcagttaag aaaaaattct aaccacacaa tttgtatatt 3240
gtttttctcc gtattatgtt aagcaaatgt tcactgcagt aaaatgtttt ggaaattagc 3300
tttgtcttat ttccagttta gttcagagaa ttaattggaa acctgatttc ttttacacat 3360
aaacctgaca aaaaatgtag cttagagcaa agggtgaatg tttgcttaac tcctgcttac 3420
ttctcaagta catgaaaact ttaatagaat atgccagtat tcactgagtt tttaaaaata 3480
ttaccatgtg taaacatata atatccaact tcatccaaaa atatggttga gtttaagtac 3540
tttgtttttc aggcttattt caagtataat aattctttga ttttcattgt tctgatttct 3600
gggtcttcaa ttcattcgtc acttttcctt tttaagtaaa ataagctttt tttttttttt 3660
tttttttttt ttggagttgc attgggattt ttcccaggaa aaaatatggc ttttagtaat 3720
gctttgcaat tggctacgca gatataaatt aagatatgtt tattctgagt tcttattgga 3780
ataagtttca aaatcaacga gcttaagaat gaaaacaaaa cttttgagag tctcacaaaa 3840
tagctttctg gtcaatacac cttacttgat ttttaagctc gcagaataaa gtatagaaac 3900
aaatggagct gaagttccat ttgctaattc agagactttt gtgcttccgc aaattggagg 3960
gcagcaagcc atcctattct catagtaatc gttttggctt tgaaatttac atacaattta 4020
atagcacatt tttagccatt atggattggc gcaataaaga gatatcaatg taatgcaatg 4080
tgatgcttta tgggcctcat tctaattcag aaagcttgtt taaaagaact aagactcttc 4140
tgtttaataa aatagcaaca atctaatatc tagattggta gtcctgcggt gccactagtg 4200
ggagatgaga gtattaagac aagagtaagg acaaggaaag acttaaaggt tgcatattga 4260
aaagtttgga attcctaatt tgggagcact gatttcttgg tgaagaagta agtatgacta 4320
cgttgccagt aattttttaa aaacatagac ccagaaatag caaatcgatt tcaccctcat 4380
accttagtct acaaggcctt gctcttgaga aggttttcca tgatattgct taatttcatc 4440
tgcacaagat gagacacaaa cataaaaatt ccctgctcat tttaatacca taaaaggctg 4500
aggttatttc tctgtcataa aattgtaaat agcatttttt aagtcaaaat tacatttaaa 4560
acagtggatt gttctacaaa tatatatgtg tatatataca tatgcttctg aaataaggat 4620
atattatatg agtttttatt tgatttgtgg tctttagtca taggtaatca aaaataaaga 4680
gatttgaatg caaaacttta tacattaatg tacatttcta atgatggtac aaattgccac 4740
tttataataa aaaagaaaca ggtgggaata ataatcaaag cacgtgttcc ttcagtactt 4800
tggtgatttt taatccccct tgtgatgcac aggaaattat tttttagtta caaaaagtta 4860
tcttagaaat ctatacttcc caatacagat ttcatgttaa gtcatatcaa attgagaatt 4920
tgtggtgaaa gaataggaaa aggatgctag atgctgatct ttctttttca ggatttttcc 4980
tggagcccaa gttaaaaatt caatacttaa atctaagtta agtgaaaatt aataatgttc 5040
agaatgatgt attgagcttt agtaacagac ggaagcaaaa aaaaataaga atatttaaca 5100
ttatgataat agccttaaaa taatgtaata aaaattgcat cattaaatgt tctattagtt 5160
ggaaagaatg agctgatgtt tctttgtctt tgctccaagt acaatttaaa gacagtgaca 5220
ttcattttac ttaaaattgt tcaaaaagtc caaaacatac tcccatggct agaattggta 5280
ttagctccaa tacaaggtta aatgttacaa tcttaagaaa ttattgacac tgaaatgttt 5340
agtaaacatg ttgtatgaga aactaaacaa attaatgttt catttttcca ttaaagcaca 5400
gattattc 5408
<210> SEQ ID NO 70
<211> LENGTH: 2705
<212> TYPE: DNA
<213> ORGANISM: Homo sapiens
<400> SEQUENCE: 70
gtgccgcgcc cagagcagca gcaacagcga agatgcgagg ccattacctg tttgatccct 60
gtcggaaacc tggcacgggc caacttttcc cgattatcac gccaagaagt tgcaaggact 120
agtcgaagac tcggaggggc cagggcgagg gcgcgctccc ccgcgcgctg cctcgtccct 180
cctccgtccg gccgcccgag ctcccggcct ctctcccgcc cgcgctcact ccctccgccc 240
gcctccctcc tctggccccc atcagaaggg caacagggcg agggggtccg gcgaaattcg 300
gaccggagca gctggacatg cacggtgtcc gccgggcgca ggggccgacc acacgcagtc 360
gcgcagttca gcatccgcgt gccagtctcg cccgcgatcc cgggcccggg gctgtggcgt 420
cgactccgac ccaggcagcc agcagcccgc gcgggagccg gaccgccgcc ggaggagctc 480
ggacggcatg ctgagccccc tccttggctg aagcccgagt gcggagaagc ccgggcaaac 540
gcaggctaag gagaccaaag cggcgaagtc gcgagacagc ggacaagcag cggaggagaa 600
ggaggaggag gcgaacccag agaggggcag caaaagaagc ggtggtggtg ggcgtcgtgg 660
ccatggcggc ggctatcgcc agctcgctca tccgtcagaa gaggcaagcc cgcgagcgcg 720
agaaatccaa cgcctgcaag tgtgtcagca gccccagcaa aggcaagacc agctgcgaca 780
aaaacaagtt aaatgtcttt tcccgggtca aactcttcgg ctccaagaag aggcgcagaa 840
gaagaccaga gcctcagctt aagggtatag ttaccaagct atacagccga caaggctacc 900
acttgcagct gcaggcggat ggaaccattg atggcaccaa agatgaggac agcacttaca 960
ctctgtttaa cctcatccct gtgggtctgc gagtggtggc tatccaagga gttcaaacca 1020
agctgtactt ggcaatgaac agtgagggat acttgtacac ctcggaactt ttcacacctg 1080
agtgcaaatt caaagaatca gtgtttgaaa attattatgt gacatattca tcaatgatat 1140
accgtcagca gcagtcaggc cgagggtggt atctgggtct gaacaaagaa ggagagatca 1200
tgaaaggcaa ccatgtgaag aagaacaagc ctgcagctca ttttctgcct aaaccactga 1260
aagtggccat gtacaaggag ccatcactgc acgatctcac ggagttctcc cgatctggaa 1320
gcgggacccc aaccaagagc agaagtgtct ctggcgtgct gaacggaggc aaatccatga 1380
gccacaatga atcaacgtag ccagtgaggg caaaagaagg gctctgtaac agaaccttac 1440
ctccaggtgc tgttgaattc ttctagcagt ccttcaccca aaagttcaaa tttgtcagtg 1500
acatttacca aacaaacagg cagagttcac tattctatct gccattagac cttcttatca 1560
tccatactaa agccccatta tttagattga gcttgtgcat aagaatgcca agcattttag 1620
tgaactaaat ctgagagaag gactgccaaa ttttctcatg atctcaccta tactttgggg 1680
atgataatcc aaaagtattt cacagcacta atgctgatca aaatttgctc tcccaccaag 1740
aaaatgtaaa agaccacaat tgttcttcaa aaacaaacaa aacaaaacaa aacaaaatta 1800
actgcttaaa tgttttgtcg gggcaaacaa aattatgtga attgtgttgt tttcttggct 1860
tgatgttttc tatctacgct tgattcacat gtactctttt ctttggcata gtgcaacttt 1920
atgatttctg aaattcaatg gttctattga ctttttgcgt cacttaatcc aaatcaacca 1980
aattcagggt tgaatctgaa ttggcttctc aggctcaagg taacagtgtt cttgtggttt 2040
gaccaattgt ttttctttct tttttttttt ttttagattt gtggtattct ggtcaagtta 2100
ttgtgctgta ctttgtgcgt agaaattgag ttgtattgtc aaccccagtc agtaaagaga 2160
acttcaaaaa attatcctca agtgtagatt tctcttaatt ccatttgtgt atcatgttaa 2220
actattgttg tggcttcttg tgtaaagaca ggaactgtgg aactgtgatg ttgtcttttg 2280
tgttgttaaa ataagaaatg tcttatctgt atatgtatga gtcttcctgt cattgtattt 2340
ggcacatgaa tattgtgtac aaggaattgt taagactggt tttccctcaa caacatatat 2400
tatacttgct actggaaaag tgtttaagac ttagctaggt ttccatttag atcttcatat 2460
ctgttgcatg gaagaaagtt gggttcttgg catagagttg catgatatgt aagattttgt 2520
gcattcataa ttgttaaaaa tctgtgttcc aaaagtggac atagcatgta caggcagttt 2580
tctgtcctgt gcacaaaaag tttaaaaaag ttgtttaata tttgttgttg tatacccaaa 2640
tacgcaccga ataaactctt tatattcatt caaagaaaaa aaaaaaaaaa aaaaaaaaaa 2700
aaaaa 2705
<210> SEQ ID NO 71
<211> LENGTH: 2340
<212> TYPE: DNA
<213> ORGANISM: Homo sapiens
<400> SEQUENCE: 71
gtggctctct aggaccggag agttctttgg aaggagagcg cgagcgaggg agcgggcgag 60
ctccgagggg gtgtgggtgt agggagagag agaaagagag caggcagcgg cggcggcggc 120
agcggtgggg aaaagcggat tccgccccga accacaccga ggggagctcg tggtcgagac 180
ttgccgccct aagcactctc ccaagtccga cccgctcggc gaggacttcc gtcttctgag 240
cgaaccttgt caagcaagct gggatctatg agtggaaagg tgaccaagcc caaagaggag 300
aaagatgctt ctaaggttct ggatgacgcc ccccctggca cacaggaata cattatgtta 360
cgacaagatt ccatccaatc tgcggaatta aagaaaaaag agtccccctt tcgtgctaag 420
tgtcacgaaa tcttctgctg cccgctgaag caagtacacc acaaagagaa cacagagccg 480
gaagagcctc agcttaaggg tatagttacc aagctataca gccgacaagg ctaccacttg 540
cagctgcagg cggatggaac cattgatggc accaaagatg aggacagcac ttacactctg 600
tttaacctca tccctgtggg tctgcgagtg gtggctatcc aaggagttca aaccaagctg 660
tacttggcaa tgaacagtga gggatacttg tacacctcgg aacttttcac acctgagtgc 720
aaattcaaag aatcagtgtt tgaaaattat tatgtgacat attcatcaat gatataccgt 780
cagcagcagt caggccgagg gtggtatctg ggtctgaaca aagaaggaga gatcatgaaa 840
ggcaaccatg tgaagaagaa caagcctgca gctcattttc tgcctaaacc actgaaagtg 900
gccatgtaca aggagccatc actgcacgat ctcacggagt tctcccgatc tggaagcggg 960
accccaacca agagcagaag tgtctctggc gtgctgaacg gaggcaaatc catgagccac 1020
aatgaatcaa cgtagccagt gagggcaaaa gaagggctct gtaacagaac cttacctcca 1080
ggtgctgttg aattcttcta gcagtccttc acccaaaagt tcaaatttgt cagtgacatt 1140
taccaaacaa acaggcagag ttcactattc tatctgccat tagaccttct tatcatccat 1200
actaaagccc cattatttag attgagcttg tgcataagaa tgccaagcat tttagtgaac 1260
taaatctgag agaaggactg ccaaattttc tcatgatctc acctatactt tggggatgat 1320
aatccaaaag tatttcacag cactaatgct gatcaaaatt tgctctccca ccaagaaaat 1380
gtaaaagacc acaattgttc ttcaaaaaca aacaaaacaa aacaaaacaa aattaactgc 1440
ttaaatgttt tgtcggggca aacaaaatta tgtgaattgt gttgttttct tggcttgatg 1500
ttttctatct acgcttgatt cacatgtact cttttctttg gcatagtgca actttatgat 1560
ttctgaaatt caatggttct attgactttt tgcgtcactt aatccaaatc aaccaaattc 1620
agggttgaat ctgaattggc ttctcaggct caaggtaaca gtgttcttgt ggtttgacca 1680
attgtttttc tttctttttt ttttttttta gatttgtggt attctggtca agttattgtg 1740
ctgtactttg tgcgtagaaa ttgagttgta ttgtcaaccc cagtcagtaa agagaacttc 1800
aaaaaattat cctcaagtgt agatttctct taattccatt tgtgtatcat gttaaactat 1860
tgttgtggct tcttgtgtaa agacaggaac tgtggaactg tgatgttgtc ttttgtgttg 1920
ttaaaataag aaatgtctta tctgtatatg tatgagtctt cctgtcattg tatttggcac 1980
atgaatattg tgtacaagga attgttaaga ctggttttcc ctcaacaaca tatattatac 2040
ttgctactgg aaaagtgttt aagacttagc taggtttcca tttagatctt catatctgtt 2100
gcatggaaga aagttgggtt cttggcatag agttgcatga tatgtaagat tttgtgcatt 2160
cataattgtt aaaaatctgt gttccaaaag tggacatagc atgtacaggc agttttctgt 2220
cctgtgcaca aaaagtttaa aaaagttgtt taatatttgt tgttgtatac ccaaatacgc 2280
accgaataaa ctctttatat tcattcaaag aaaaaaaaaa aaaaaaaaaa aaaaaaaaaa 2340
<210> SEQ ID NO 72
<211> LENGTH: 2450
<212> TYPE: DNA
<213> ORGANISM: Homo sapiens
<400> SEQUENCE: 72
gtggctctct aggaccggag agttctttgg aaggagagcg cgagcgaggg agcgggcgag 60
ctccgagggg gtgtgggtgt agggagagag agaaagagag caggcagcgg cggcggcggc 120
agcggtgggg aaaagcggat tccgccccga accacaccga ggggagctcg tggtcgagac 180
ttgccgccct aagcactctc ccaagtccga cccgctcggc gaggacttcc gtcttctgag 240
cgaaccttgt caagcaagct gggatctatg agtggaaagg tgaccaagcc caaagaggag 300
aaagatgctt ctaagggagt ttctctgcac aagctctctg tttgcctgct gtcgtccaca 360
taagatgtga cttgctcctg cttgccttcc tccatgattg tgaggcctcc ccagccacgt 420
ggaactttct ggatgacgcc ccccctggca cacaggaata cattatgtta cgacaagatt 480
ccatccaatc tgcggaatta aagaaaaaag agtccccctt tcgtgctaag tgtcacgaaa 540
tcttctgctg cccgctgaag caagtacacc acaaagagaa cacagagccg gaagagcctc 600
agcttaaggg tatagttacc aagctataca gccgacaagg ctaccacttg cagctgcagg 660
cggatggaac cattgatggc accaaagatg aggacagcac ttacactctg tttaacctca 720
tccctgtggg tctgcgagtg gtggctatcc aaggagttca aaccaagctg tacttggcaa 780
tgaacagtga gggatacttg tacacctcgg aacttttcac acctgagtgc aaattcaaag 840
aatcagtgtt tgaaaattat tatgtgacat attcatcaat gatataccgt cagcagcagt 900
caggccgagg gtggtatctg ggtctgaaca aagaaggaga gatcatgaaa ggcaaccatg 960
tgaagaagaa caagcctgca gctcattttc tgcctaaacc actgaaagtg gccatgtaca 1020
aggagccatc actgcacgat ctcacggagt tctcccgatc tggaagcggg accccaacca 1080
agagcagaag tgtctctggc gtgctgaacg gaggcaaatc catgagccac aatgaatcaa 1140
cgtagccagt gagggcaaaa gaagggctct gtaacagaac cttacctcca ggtgctgttg 1200
aattcttcta gcagtccttc acccaaaagt tcaaatttgt cagtgacatt taccaaacaa 1260
acaggcagag ttcactattc tatctgccat tagaccttct tatcatccat actaaagccc 1320
cattatttag attgagcttg tgcataagaa tgccaagcat tttagtgaac taaatctgag 1380
agaaggactg ccaaattttc tcatgatctc acctatactt tggggatgat aatccaaaag 1440
tatttcacag cactaatgct gatcaaaatt tgctctccca ccaagaaaat gtaaaagacc 1500
acaattgttc ttcaaaaaca aacaaaacaa aacaaaacaa aattaactgc ttaaatgttt 1560
tgtcggggca aacaaaatta tgtgaattgt gttgttttct tggcttgatg ttttctatct 1620
acgcttgatt cacatgtact cttttctttg gcatagtgca actttatgat ttctgaaatt 1680
caatggttct attgactttt tgcgtcactt aatccaaatc aaccaaattc agggttgaat 1740
ctgaattggc ttctcaggct caaggtaaca gtgttcttgt ggtttgacca attgtttttc 1800
tttctttttt ttttttttta gatttgtggt attctggtca agttattgtg ctgtactttg 1860
tgcgtagaaa ttgagttgta ttgtcaaccc cagtcagtaa agagaacttc aaaaaattat 1920
cctcaagtgt agatttctct taattccatt tgtgtatcat gttaaactat tgttgtggct 1980
tcttgtgtaa agacaggaac tgtggaactg tgatgttgtc ttttgtgttg ttaaaataag 2040
aaatgtctta tctgtatatg tatgagtctt cctgtcattg tatttggcac atgaatattg 2100
tgtacaagga attgttaaga ctggttttcc ctcaacaaca tatattatac ttgctactgg 2160
aaaagtgttt aagacttagc taggtttcca tttagatctt catatctgtt gcatggaaga 2220
aagttgggtt cttggcatag agttgcatga tatgtaagat tttgtgcatt cataattgtt 2280
aaaaatctgt gttccaaaag tggacatagc atgtacaggc agttttctgt cctgtgcaca 2340
aaaagtttaa aaaagttgtt taatatttgt tgttgtatac ccaaatacgc accgaataaa 2400
ctctttatat tcattcaaag aaaaaaaaaa aaaaaaaaaa aaaaaaaaaa 2450
<210> SEQ ID NO 73
<211> LENGTH: 2172
<212> TYPE: DNA
<213> ORGANISM: Homo sapiens
<400> SEQUENCE: 73
gtggctctct aggaccggag agttctttgg aaggagagcg cgagcgaggg agcgggcgag 60
ctccgagggg gtgtgggtgt agggagagag agaaagagag caggcagcgg cggcggcggc 120
agcggtgggg aaaagcggat tccgccccga accacaccga ggggagctcg tggtcgagac 180
ttgccgccct aagcactctc ccaagtccga cccgctcggc gaggacttcc gtcttctgag 240
cgaaccttgt caagcaagct gggatctatg agtggaaagg tgaccaagcc caaagaggag 300
aaagatgctt ctaaggagcc tcagcttaag ggtatagtta ccaagctata cagccgacaa 360
ggctaccact tgcagctgca ggcggatgga accattgatg gcaccaaaga tgaggacagc 420
acttacactc tgtttaacct catccctgtg ggtctgcgag tggtggctat ccaaggagtt 480
caaaccaagc tgtacttggc aatgaacagt gagggatact tgtacacctc ggaacttttc 540
acacctgagt gcaaattcaa agaatcagtg tttgaaaatt attatgtgac atattcatca 600
atgatatacc gtcagcagca gtcaggccga gggtggtatc tgggtctgaa caaagaagga 660
gagatcatga aaggcaacca tgtgaagaag aacaagcctg cagctcattt tctgcctaaa 720
ccactgaaag tggccatgta caaggagcca tcactgcacg atctcacgga gttctcccga 780
tctggaagcg ggaccccaac caagagcaga agtgtctctg gcgtgctgaa cggaggcaaa 840
tccatgagcc acaatgaatc aacgtagcca gtgagggcaa aagaagggct ctgtaacaga 900
accttacctc caggtgctgt tgaattcttc tagcagtcct tcacccaaaa gttcaaattt 960
gtcagtgaca tttaccaaac aaacaggcag agttcactat tctatctgcc attagacctt 1020
cttatcatcc atactaaagc cccattattt agattgagct tgtgcataag aatgccaagc 1080
attttagtga actaaatctg agagaaggac tgccaaattt tctcatgatc tcacctatac 1140
tttggggatg ataatccaaa agtatttcac agcactaatg ctgatcaaaa tttgctctcc 1200
caccaagaaa atgtaaaaga ccacaattgt tcttcaaaaa caaacaaaac aaaacaaaac 1260
aaaattaact gcttaaatgt tttgtcgggg caaacaaaat tatgtgaatt gtgttgtttt 1320
cttggcttga tgttttctat ctacgcttga ttcacatgta ctcttttctt tggcatagtg 1380
caactttatg atttctgaaa ttcaatggtt ctattgactt tttgcgtcac ttaatccaaa 1440
tcaaccaaat tcagggttga atctgaattg gcttctcagg ctcaaggtaa cagtgttctt 1500
gtggtttgac caattgtttt tctttctttt tttttttttt tagatttgtg gtattctggt 1560
caagttattg tgctgtactt tgtgcgtaga aattgagttg tattgtcaac cccagtcagt 1620
aaagagaact tcaaaaaatt atcctcaagt gtagatttct cttaattcca tttgtgtatc 1680
atgttaaact attgttgtgg cttcttgtgt aaagacagga actgtggaac tgtgatgttg 1740
tcttttgtgt tgttaaaata agaaatgtct tatctgtata tgtatgagtc ttcctgtcat 1800
tgtatttggc acatgaatat tgtgtacaag gaattgttaa gactggtttt ccctcaacaa 1860
catatattat acttgctact ggaaaagtgt ttaagactta gctaggtttc catttagatc 1920
ttcatatctg ttgcatggaa gaaagttggg ttcttggcat agagttgcat gatatgtaag 1980
attttgtgca ttcataattg ttaaaaatct gtgttccaaa agtggacata gcatgtacag 2040
gcagttttct gtcctgtgca caaaaagttt aaaaaagttg tttaatattt gttgttgtat 2100
acccaaatac gcaccgaata aactctttat attcattcaa agaaaaaaaa aaaaaaaaaa 2160
aaaaaaaaaa aa 2172
<210> SEQ ID NO 74
<211> LENGTH: 2093
<212> TYPE: DNA
<213> ORGANISM: Homo sapiens
<400> SEQUENCE: 74
catgtaacat gtgatttgct cctccttgcc ttccaccgtg atgtgaggcc tccccaacca 60
agtggaactt tctggatgac gccccccctg gcacacagga atacattatg ttacgacaag 120
attccatcca atctgcggaa ttaaagaaaa aagagtcccc ctttcgtgct aagtgtcacg 180
aaatcttctg ctgcccgctg aagcaagtac accacaaaga gaacacagag ccggaagagc 240
ctcagcttaa gggtatagtt accaagctat acagccgaca aggctaccac ttgcagctgc 300
aggcggatgg aaccattgat ggcaccaaag atgaggacag cacttacact ctgtttaacc 360
tcatccctgt gggtctgcga gtggtggcta tccaaggagt tcaaaccaag ctgtacttgg 420
caatgaacag tgagggatac ttgtacacct cggaactttt cacacctgag tgcaaattca 480
aagaatcagt gtttgaaaat tattatgtga catattcatc aatgatatac cgtcagcagc 540
agtcaggccg agggtggtat ctgggtctga acaaagaagg agagatcatg aaaggcaacc 600
atgtgaagaa gaacaagcct gcagctcatt ttctgcctaa accactgaaa gtggccatgt 660
acaaggagcc atcactgcac gatctcacgg agttctcccg atctggaagc gggaccccaa 720
ccaagagcag aagtgtctct ggcgtgctga acggaggcaa atccatgagc cacaatgaat 780
caacgtagcc agtgagggca aaagaagggc tctgtaacag aaccttacct ccaggtgctg 840
ttgaattctt ctagcagtcc ttcacccaaa agttcaaatt tgtcagtgac atttaccaaa 900
caaacaggca gagttcacta ttctatctgc cattagacct tcttatcatc catactaaag 960
ccccattatt tagattgagc ttgtgcataa gaatgccaag cattttagtg aactaaatct 1020
gagagaagga ctgccaaatt ttctcatgat ctcacctata ctttggggat gataatccaa 1080
aagtatttca cagcactaat gctgatcaaa atttgctctc ccaccaagaa aatgtaaaag 1140
accacaattg ttcttcaaaa acaaacaaaa caaaacaaaa caaaattaac tgcttaaatg 1200
ttttgtcggg gcaaacaaaa ttatgtgaat tgtgttgttt tcttggcttg atgttttcta 1260
tctacgcttg attcacatgt actcttttct ttggcatagt gcaactttat gatttctgaa 1320
attcaatggt tctattgact ttttgcgtca cttaatccaa atcaaccaaa ttcagggttg 1380
aatctgaatt ggcttctcag gctcaaggta acagtgttct tgtggtttga ccaattgttt 1440
ttctttcttt tttttttttt ttagatttgt ggtattctgg tcaagttatt gtgctgtact 1500
ttgtgcgtag aaattgagtt gtattgtcaa ccccagtcag taaagagaac ttcaaaaaat 1560
tatcctcaag tgtagatttc tcttaattcc atttgtgtat catgttaaac tattgttgtg 1620
gcttcttgtg taaagacagg aactgtggaa ctgtgatgtt gtcttttgtg ttgttaaaat 1680
aagaaatgtc ttatctgtat atgtatgagt cttcctgtca ttgtatttgg cacatgaata 1740
ttgtgtacaa ggaattgtta agactggttt tccctcaaca acatatatta tacttgctac 1800
tggaaaagtg tttaagactt agctaggttt ccatttagat cttcatatct gttgcatgga 1860
agaaagttgg gttcttggca tagagttgca tgatatgtaa gattttgtgc attcataatt 1920
gttaaaaatc tgtgttccaa aagtggacat agcatgtaca ggcagttttc tgtcctgtgc 1980
acaaaaagtt taaaaaagtt gtttaatatt tgttgttgta tacccaaata cgcaccgaat 2040
aaactcttta tattcattca aagaaaaaaa aaaaaaaaaa aaaaaaaaaa aaa 2093
<210> SEQ ID NO 75
<211> LENGTH: 1968
<212> TYPE: DNA
<213> ORGANISM: Homo sapiens
<400> SEQUENCE: 75
aaactttctc tgatctcctc tctctctgtg tctgctccaa atgtagacag caattgtctg 60
ggtaggacca gcttataaag aagcatggct ttgttaagga agtcgtattc agagcctcag 120
cttaagggta tagttaccaa gctatacagc cgacaaggct accacttgca gctgcaggcg 180
gatggaacca ttgatggcac caaagatgag gacagcactt acactctgtt taacctcatc 240
cctgtgggtc tgcgagtggt ggctatccaa ggagttcaaa ccaagctgta cttggcaatg 300
aacagtgagg gatacttgta cacctcggaa cttttcacac ctgagtgcaa attcaaagaa 360
tcagtgtttg aaaattatta tgtgacatat tcatcaatga tataccgtca gcagcagtca 420
ggccgagggt ggtatctggg tctgaacaaa gaaggagaga tcatgaaagg caaccatgtg 480
aagaagaaca agcctgcagc tcattttctg cctaaaccac tgaaagtggc catgtacaag 540
gagccatcac tgcacgatct cacggagttc tcccgatctg gaagcgggac cccaaccaag 600
agcagaagtg tctctggcgt gctgaacgga ggcaaatcca tgagccacaa tgaatcaacg 660
tagccagtga gggcaaaaga agggctctgt aacagaacct tacctccagg tgctgttgaa 720
ttcttctagc agtccttcac ccaaaagttc aaatttgtca gtgacattta ccaaacaaac 780
aggcagagtt cactattcta tctgccatta gaccttctta tcatccatac taaagcccca 840
ttatttagat tgagcttgtg cataagaatg ccaagcattt tagtgaacta aatctgagag 900
aaggactgcc aaattttctc atgatctcac ctatactttg gggatgataa tccaaaagta 960
tttcacagca ctaatgctga tcaaaatttg ctctcccacc aagaaaatgt aaaagaccac 1020
aattgttctt caaaaacaaa caaaacaaaa caaaacaaaa ttaactgctt aaatgttttg 1080
tcggggcaaa caaaattatg tgaattgtgt tgttttcttg gcttgatgtt ttctatctac 1140
gcttgattca catgtactct tttctttggc atagtgcaac tttatgattt ctgaaattca 1200
atggttctat tgactttttg cgtcacttaa tccaaatcaa ccaaattcag ggttgaatct 1260
gaattggctt ctcaggctca aggtaacagt gttcttgtgg tttgaccaat tgtttttctt 1320
tctttttttt tttttttaga tttgtggtat tctggtcaag ttattgtgct gtactttgtg 1380
cgtagaaatt gagttgtatt gtcaacccca gtcagtaaag agaacttcaa aaaattatcc 1440
tcaagtgtag atttctctta attccatttg tgtatcatgt taaactattg ttgtggcttc 1500
ttgtgtaaag acaggaactg tggaactgtg atgttgtctt ttgtgttgtt aaaataagaa 1560
atgtcttatc tgtatatgta tgagtcttcc tgtcattgta tttggcacat gaatattgtg 1620
tacaaggaat tgttaagact ggttttccct caacaacata tattatactt gctactggaa 1680
aagtgtttaa gacttagcta ggtttccatt tagatcttca tatctgttgc atggaagaaa 1740
gttgggttct tggcatagag ttgcatgata tgtaagattt tgtgcattca taattgttaa 1800
aaatctgtgt tccaaaagtg gacatagcat gtacaggcag ttttctgtcc tgtgcacaaa 1860
aagtttaaaa aagttgttta atatttgttg ttgtataccc aaatacgcac cgaataaact 1920
ctttatattc attcaaagaa aaaaaaaaaa aaaaaaaaaa aaaaaaaa 1968
<210> SEQ ID NO 76
<211> LENGTH: 2720
<212> TYPE: DNA
<213> ORGANISM: Homo sapiens
<400> SEQUENCE: 76
atggccgcgg ccatcgctag cggcttgatc cgccagaagc ggcaggcgcg ggagcagcac 60
tgggaccggc cgtctgccag caggaggcgg agcagcccca gcaagaaccg cgggctctgc 120
aacggcaacc tggtggatat cttctccaaa gtgcgcatct tcggcctcaa gaagcgcagg 180
ttgcggcgcc aagatcccca gctcaagggt atagtgacca ggttatattg caggcaaggc 240
tactacttgc aaatgcaccc cgatggagct ctcgatggaa ccaaggatga cagcactaat 300
tctacactct tcaacctcat accagtggga ctacgtgttg ttgccatcca gggagtgaaa 360
acagggttgt atatagccat gaatggagaa ggttacctct acccatcaga actttttacc 420
cctgaatgca agtttaaaga atctgttttt gaaaattatt atgtaatcta ctcatccatg 480
ttgtacagac aacaggaatc tggtagagcc tggtttttgg gattaaataa ggaagggcaa 540
gctatgaaag ggaacagagt aaagaaaacc aaaccagcag ctcattttct acccaagcca 600
ttggaagttg ccatgtaccg agaaccatct ttgcatgatg ttggggaaac ggtcccgaag 660
cctggggtga cgccaagtaa aagcacaagt gcgtctgcaa taatgaatgg aggcaaacca 720
gtcaacaaga gtaagacaac atagccagat cctcacaggt gttgtgactt attcgtcctg 780
agcacagttg agtgatttat cctcaccaga cattcctgct ccgtggctga agagcagcag 840
gaagtaagct aatgcttatt ctttgctgtc tccgaacttc tctgttgcaa gtggataaat 900
ctcaacctgt tgcacccccc acaacaagaa gacacctgga taaccagcta aactcagacc 960
atggaatgcc ctaccagata tggaatgcct ttttaatatc ttttctgtga ctgtgacact 1020
tcatgtgaat gacatacttc acaagtacac tcgatacctt gcctgctgac agctacccat 1080
aatccttttt gagtcctgtt tcagcgaaat ctatgtgttt aagttcaatt ttgtagcaca 1140
caaataatat tgagtaattt ctagttagac gctgtaaacc tgtgctatta cggatttctc 1200
ttcttcccat ttttacaggg ctgctcgctc cactgtctgt gaccttttgc agggattttg 1260
ttcctctaaa tcttaaatgt tgcagttggc ttaggtcgga gagcaatcag ggaatcagga 1320
agccttctaa acctattatt acaaattgca tctataaaga aagattaaga aagattgttg 1380
tctctggctc acactatcga ttaaacacac atatacgctc tgtccagtag cagatactgt 1440
gctcccaagg tcggcattgc ctgggtggga aatggctcaa acacaatcca gggaagctct 1500
ctatgatatg tgtttgacat ccccctctag tttctttgtg tgtgtgtgtt ttatacatat 1560
cacaagctta ctggtaatgg taacatttgc cttgcccagc gagcaagacc cactggtttt 1620
tgagaaagtg ggtccaaaga tttctgtagg ccttgtaggc ctgattaagg ttcatttttc 1680
atctattaat tctcattatt tggaaaaaaa aaaaaaggaa aatcagtaat tataacctac 1740
aagaattgcg ctacctaaat ccatttcaga tatactccgt cctgttttta atgaaccaaa 1800
cttaacgcca tccccgtttc tggctgcgtt cccctcatac tcagcagagc atgggcaaga 1860
cggctgttgt gttctttcct gcagcagcaa tgcaaacgtt agttataaat taattagact 1920
ttaatatttt tggtgtttaa tgacaagttt ttaaactgga catattagga aaaatatttt 1980
ttttagctca gcatgctgag tccggtactg tgtatttcac cagtacatgc ctctagctca 2040
gcatctgggg ctcatgttgc ccagtggctg ggttagaggt gccttgccat gatctcagaa 2100
tacagtctgt tgaattatcc tagatgaaaa taaaggcaaa ccaacacatt catccatgag 2160
gattttggtc cattccattt attttctttt attttgcatt cttaatttcc tttttagttt 2220
aacactgttt gtttgagctt agggaagaca actaccaaga aaggccagga acagttgact 2280
acacaatgaa gattccatgc aaaatgttca atattggatc taaaggggtt caaaatgttt 2340
catactaaac tgtttgggaa tttatttgtt aactctgtgt acacctaata aaattcaatg 2400
ttttcttctc agaagagttc attgagacca aactgaacct catttattga aaattatatg 2460
tgggatcaat gtactggcct cttgttattc tttctatgtg ggaggatgac ccagtcatca 2520
ttttccccat ctgcactgta tttattggga aattattttg tcactgcttt cataaatctt 2580
cttcatgaca gcccttgccc agcattaaaa aattctggcc tgcttagctg attaaaggtt 2640
tagtagaaat ttaactgttt gtttatgctt atttcatttt catattggat tctacttgaa 2700
taaataaaaa gttagcagaa 2720
<210> SEQ ID NO 77
<211> LENGTH: 2831
<212> TYPE: DNA
<213> ORGANISM: Homo sapiens
<400> SEQUENCE: 77
ctggccgaaa acaaacaatc actgagaagt ctcaaagaaa tataccacgt gaggggaaaa 60
aactgggaga agatccggaa tattatcgtt tttcctatgg taaaaccggt gcccctcttc 120
aggagaactg atttcaaatt attattatgc aaccacaagg atctcttctt tctcagggtg 180
tctaagctgc tggattgctt ttcgcccaaa tcaatgtggt ttctttggaa cattttcagc 240
aaaggaacgc atatgctgca gtgtctttgt ggcaagagtc ttaagaaaaa caagaaccca 300
actgatcccc agctcaaggg tatagtgacc aggttatatt gcaggcaagg ctactacttg 360
caaatgcacc ccgatggagc tctcgatgga accaaggatg acagcactaa ttctacactc 420
ttcaacctca taccagtggg actacgtgtt gttgccatcc agggagtgaa aacagggttg 480
tatatagcca tgaatggaga aggttacctc tacccatcag aactttttac ccctgaatgc 540
aagtttaaag aatctgtttt tgaaaattat tatgtaatct actcatccat gttgtacaga 600
caacaggaat ctggtagagc ctggtttttg ggattaaata aggaagggca agctatgaaa 660
gggaacagag taaagaaaac caaaccagca gctcattttc tacccaagcc attggaagtt 720
gccatgtacc gagaaccatc tttgcatgat gttggggaaa cggtcccgaa gcctggggtg 780
acgccaagta aaagcacaag tgcgtctgca ataatgaatg gaggcaaacc agtcaacaag 840
agtaagacaa catagccaga tcctcacagg tgttgtgact tattcgtcct gagcacagtt 900
gagtgattta tcctcaccag acattcctgc tccgtggctg aagagcagca ggaagtaagc 960
taatgcttat tctttgctgt ctccgaactt ctctgttgca agtggataaa tctcaacctg 1020
ttgcaccccc cacaacaaga agacacctgg ataaccagct aaactcagac catggaatgc 1080
cctaccagat atggaatgcc tttttaatat cttttctgtg actgtgacac ttcatgtgaa 1140
tgacatactt cacaagtaca ctcgatacct tgcctgctga cagctaccca taatcctttt 1200
tgagtcctgt ttcagcgaaa tctatgtgtt taagttcaat tttgtagcac acaaataata 1260
ttgagtaatt tctagttaga cgctgtaaac ctgtgctatt acggatttct cttcttccca 1320
tttttacagg gctgctcgct ccactgtctg tgaccttttg cagggatttt gttcctctaa 1380
atcttaaatg ttgcagttgg cttaggtcgg agagcaatca gggaatcagg aagccttcta 1440
aacctattat tacaaattgc atctataaag aaagattaag aaagattgtt gtctctggct 1500
cacactatcg attaaacaca catatacgct ctgtccagta gcagatactg tgctcccaag 1560
gtcggcattg cctgggtggg aaatggctca aacacaatcc agggaagctc tctatgatat 1620
gtgtttgaca tccccctcta gtttctttgt gtgtgtgtgt tttatacata tcacaagctt 1680
actggtaatg gtaacatttg ccttgcccag cgagcaagac ccactggttt ttgagaaagt 1740
gggtccaaag atttctgtag gccttgtagg cctgattaag gttcattttt catctattaa 1800
ttctcattat ttggaaaaaa aaaaaaagga aaatcagtaa ttataaccta caagaattgc 1860
gctacctaaa tccatttcag atatactccg tcctgttttt aatgaaccaa acttaacgcc 1920
atccccgttt ctggctgcgt tcccctcata ctcagcagag catgggcaag acggctgttg 1980
tgttctttcc tgcagcagca atgcaaacgt tagttataaa ttaattagac tttaatattt 2040
ttggtgttta atgacaagtt tttaaactgg acatattagg aaaaatattt tttttagctc 2100
agcatgctga gtccggtact gtgtatttca ccagtacatg cctctagctc agcatctggg 2160
gctcatgttg cccagtggct gggttagagg tgccttgcca tgatctcaga atacagtctg 2220
ttgaattatc ctagatgaaa ataaaggcaa accaacacat tcatccatga ggattttggt 2280
ccattccatt tattttcttt tattttgcat tcttaatttc ctttttagtt taacactgtt 2340
tgtttgagct tagggaagac aactaccaag aaaggccagg aacagttgac tacacaatga 2400
agattccatg caaaatgttc aatattggat ctaaaggggt tcaaaatgtt tcatactaaa 2460
ctgtttggga atttatttgt taactctgtg tacacctaat aaaattcaat gttttcttct 2520
cagaagagtt cattgagacc aaactgaacc tcatttattg aaaattatat gtgggatcaa 2580
tgtactggcc tcttgttatt ctttctatgt gggaggatga cccagtcatc attttcccca 2640
tctgcactgt atttattggg aaattatttt gtcactgctt tcataaatct tcttcatgac 2700
agcccttgcc cagcattaaa aaattctggc ctgcttagct gattaaaggt ttagtagaaa 2760
tttaactgtt tgtttatgct tatttcattt tcatattgga ttctacttga ataaataaaa 2820
agttagcaga a 2831
<210> SEQ ID NO 78
<211> LENGTH: 624
<212> TYPE: DNA
<213> ORGANISM: Homo sapiens
<400> SEQUENCE: 78
atggcagagg tggggggcgt cttcgcctcc ttggactggg atctacacgg cttctcctcg 60
tctctgggga acgtgccctt agctgactcc ccaggtttcc tgaacgagcg cctgggccaa 120
atcgagggga agctgcagcg tggctcaccc acagacttcg cccacctgaa ggggatcctg 180
cggcgccgcc agctctactg ccgcaccggc ttccacctgg agatcttccc caacggcacg 240
gtgcacggga cccgccacga ccacagccgc ttcggaatcc tggagtttat cagcctggct 300
gtggggctga tcagcatccg gggagtggac tctggcctgt acctaggaat gaatgagcga 360
ggagaactct atgggtcgaa gaaactcaca cgtgaatgtg ttttccggga acagtttgaa 420
gaaaactggt acaacaccta tgcctcaacc ttgtacaaac attcggactc agagagacag 480
tattacgtgg ccctgaacaa agatggctca ccccgggagg gatacaggac taaacgacac 540
cagaaattca ctcacttttt acccaggcct gtagatcctt ctaagttgcc ctccatgtcc 600
agagacctct ttcactatag gtaa 624
<210> SEQ ID NO 79
<211> LENGTH: 1238
<212> TYPE: DNA
<213> ORGANISM: Homo sapiens
<400> SEQUENCE: 79
acctctccag cgatgggagc cgcccgcctg ctgcccaacc tcactctgtg cttacagctg 60
ctgattctct gctgtcaaac tcagggggag aatcacccgt ctcctaattt taaccagtac 120
gtgagggacc agggcgccat gaccgaccag ctgagcaggc ggcagatccg cgagtaccaa 180
ctctacagca ggaccagtgg caagcacgtg caggtcaccg ggcgtcgcat ctccgccacc 240
gccgaggacg gcaacaagtt tgccaagctc atagtggaga cggacacgtt tggcagccgg 300
gttcgcatca aaggggctga gagtgagaag tacatctgta tgaacaagag gggcaagctc 360
atcgggaagc ccagcgggaa gagcaaagac tgcgtgttca cggagatcgt gctggagaac 420
aactatacgg ccttccagaa cgcccggcac gagggctggt tcatggcctt cacgcggcag 480
gggcggcccc gccaggcttc ccgcagccgc cagaaccagc gcgaggccca cttcatcaag 540
cgcctctacc aaggccagct gcccttcccc aaccacgccg agaagcagaa gcagttcgag 600
tttgtgggct ccgcccccac ccgccggacc aagcgcacac ggcggcccca gcccctcacg 660
tagtctggga ggcagggggc agcagcccct gggccgcctc cccacccctt tcccttctta 720
atccaaggac tgggctgggg tggcgggagg ggagccagat ccccgaggga ggaccctgag 780
ggccgcgaag catccgagcc cccagctggg aaggggcagg ccggtgcccc aggggcggct 840
ggcacagtgc ccccttcccg gacgggtggc aggccctgga gaggaactga gtgtcaccct 900
gatctcaggc caccagcctc tgccggcctc ccagccgggc tcctgaagcc cgctgaaagg 960
tcagcgactg aaggccttgc agacaaccgt ctggaggtgg ctgtcctcaa aatctgcttc 1020
tcggatctcc ctcagtctgc ccccagcccc caaactcctc ctggctagac tgtaggaagg 1080
gacttttgtt tgtttgtttg tttcaggaaa aaagaaaggg agagagagga aaatagaggg 1140
ttgtccactc ctcacattcc acgacccagg cctgcacccc acccccaact cccagccccg 1200
gaataaaacc attttcctgc aaaaaaaaaa aaaaaaaa 1238
<210> SEQ ID NO 80
<211> LENGTH: 1999
<212> TYPE: DNA
<213> ORGANISM: Homo sapiens
<400> SEQUENCE: 80
cacggccgga gagacgcgga ggaggagaca tgagccggcg ggcgcccaga cggagcggcc 60
gtgacgcttt cgcgctgcag ccgcgcgccc cgaccccgga gcgctgaccc ctggccccac 120
gcagctccgc gcccgggccg gagagcgcaa ctcggcttcc agacccgccg cgcatgctgt 180
ccccggactg agccgggcag ccagcctccc acggacgccc ggacggccgg ccggccagca 240
gtgagcgagc ttccccgcac cggccaggcg cctcctgcac agcggctgcc gccccgcagc 300
ccctgcgcca gcccggaggg cgcagcgctc gggaggagcc gcgcggggcg ctgatgccgc 360
agggcgcgcc gcggagcgcc ccggagcagc agagtctgca gcagcagcag ccggcgagga 420
gggagcagca gcagcggcgg cggcggcggc ggcggcggcg gaggcgcccg gtcccggccg 480
cgcggagcgg acatgtgcag gctgggctag gagccgccgc ctccctcccg cccagcgatg 540
tattcagcgc cctccgcctg cacttgcctg tgtttacact tcctgctgct gtgcttccag 600
gtacaggtgc tggttgccga ggagaacgtg gacttccgca tccacgtgga gaaccagacg 660
cgggctcggg acgatgtgag ccgtaagcag ctgcggctgt accagctcta cagccggacc 720
agtgggaaac acatccaggt cctgggccgc aggatcagtg cccgcggcga ggatggggac 780
aagtatgccc agctcctagt ggagacagac accttcggta gtcaagtccg gatcaagggc 840
aaggagacgg aattctacct gtgcatgaac cgcaaaggca agctcgtggg gaagcccgat 900
ggcaccagca aggagtgtgt gttcatcgag aaggttctgg agaacaacta cacggccctg 960
atgtcggcta agtactccgg ctggtacgtg ggcttcacca agaaggggcg gccgcggaag 1020
ggccccaaga cccgggagaa ccagcaggac gtgcatttca tgaagcgcta ccccaagggg 1080
cagccggagc ttcagaagcc cttcaagtac acgacggtga ccaagaggtc ccgtcggatc 1140
cggcccacac accctgccta ggccaccccg ccgcggcccc tcaggtcgcc ctggccacac 1200
tcacactccc agaaaactgc atcagaggaa tatttttaca tgaaaaataa ggaagaagct 1260
ctatttttgt acattgtgtt taaaagaaga caaaaactga accaaaactc ttggggggag 1320
gggtgataag gattttattg ttgacttgaa acccccgatg acaaaagact cacgcaaagg 1380
gactgtagtc aacccacagg tgcttgtctc tctctaggaa cagacaactc taaactcgtc 1440
cccagaggag gacttgaatg aggaaaccaa cactttgaga aaccaaagtc ctttttccca 1500
aaggttctga aaggaaaaaa aaaaaaaaca aaaaaaaaga aaaacaaaga gaaagtagta 1560
ctccgcccac caacaaactc cccctaactt tcccaatcct ctgttcctgc cccaaactcc 1620
aacaaaaatc gctctctggt ttgcagtcat ttatttattg tccgctgcaa gctgccccga 1680
gacaccgcgc agggaaggcg tgcccctggg aattctccgc gcctcgacct cccgacgaca 1740
gacgcctcgt ccaatcatgg tgaccctgcc ttgctcgcag ttctggagga tgctgctatc 1800
gaccttccgt gactcacgtg acctagtaca ccaatgataa gggaatattt taaaaccagc 1860
tatattatat atattatata tatataagct atttatttca cctctctgta tattgcagtt 1920
tcatgaacca agtattactg cctcaacaat taaaaacaac agacaaatta tttaaaaaac 1980
caaaaaaaaa aaaaaaaaa 1999
<210> SEQ ID NO 81
<211> LENGTH: 2157
<212> TYPE: DNA
<213> ORGANISM: Homo sapiens
<400> SEQUENCE: 81
gctcccagcc aagaacctcg gggccgctgc gcggtgggga ggagttcccc gaaacccggc 60
cgctaagcga ggcctcctcc tcccgcagat ccgaacggcc tgggcggggt caccccggct 120
gggacaagaa gccgccgcct gcctgcccgg gcccggggag ggggctgggg ctggggccgg 180
aggcggggtg tgagtgggtg tgtgcggggg gcggaggctt gatgcaatcc cgataagaaa 240
tgctcgggtg tcttgggcac ctacccgtgg ggcccgtaag gcgctactat ataaggctgc 300
cggcccggag ccgccgcgcc gtcagagcag gagcgctgcg tccaggatct agggccacga 360
ccatcccaac ccggcactca cagccccgca gcgcatcccg gtcgccgccc agcctcccgc 420
acccccatcg ccggagctgc gccgagagcc ccagggaggt gccatgcgga gcgggtgtgt 480
ggtggtccac gtatggatcc tggccggcct ctggctggcc gtggccgggc gccccctcgc 540
cttctcggac gcggggcccc acgtgcacta cggctggggc gaccccatcc gcctgcggca 600
cctgtacacc tccggccccc acgggctctc cagctgcttc ctgcgcatcc gtgccgacgg 660
cgtcgtggac tgcgcgcggg gccagagcgc gcacagtttg ctggagatca aggcagtcgc 720
tctgcggacc gtggccatca agggcgtgca cagcgtgcgg tacctctgca tgggcgccga 780
cggcaagatg caggggctgc ttcagtactc ggaggaagac tgtgctttcg aggaggagat 840
ccgcccagat ggctacaatg tgtaccgatc cgagaagcac cgcctcccgg tctccctgag 900
cagtgccaaa cagcggcagc tgtacaagaa cagaggcttt cttccactct ctcatttcct 960
gcccatgctg cccatggtcc cagaggagcc tgaggacctc aggggccact tggaatctga 1020
catgttctct tcgcccctgg agaccgacag catggaccca tttgggcttg tcaccggact 1080
ggaggccgtg aggagtccca gctttgagaa gtaactgaga ccatgcccgg gcctcttcac 1140
tgctgccagg ggctgtggta cctgcagcgt gggggacgtg cttctacaag aacagtcctg 1200
agtccacgtt ctgtttagct ttaggaagaa acatctagaa gttgtacata ttcagagttt 1260
tccattggca gtgccagttt ctagccaata gacttgtctg atcataacat tgtaagcctg 1320
tagcttgccc agctgctgcc tgggccccca ttctgctccc tcgaggttgc tggacaagct 1380
gctgcactgt ctcagttctg cttgaatacc tccatcgatg gggaactcac ttcctttgga 1440
aaaattctta tgtcaagctg aaattctcta attttttctc atcacttccc caggagcagc 1500
cagaagacag gcagtagttt taatttcagg aacaggtgat ccactctgta aaacagcagg 1560
taaatttcac tcaaccccat gtgggaattg atctatatct ctacttccag ggaccatttg 1620
cccttcccaa atccctccag gccagaactg actggagcag gcatggccca ccaggcttca 1680
ggagtagggg aagcctggag ccccactcca gccctgggac aacttgagaa ttccccctga 1740
ggccagttct gtcatggatg ctgtcctgag aataacttgc tgtcccggtg tcacctgctt 1800
ccatctccca gcccaccagc cctctgccca cctcacatgc ctccccatgg attggggcct 1860
cccaggcccc ccaccttatg tcaacctgca cttcttgttc aaaaatcagg aaaagaaaag 1920
atttgaagac cccaagtctt gtcaataact tgctgtgtgg aagcagcggg ggaagaccta 1980
gaaccctttc cccagcactt ggttttccaa catgatattt atgagtaatt tattttgata 2040
tgtacatctc ttattttctt acattattta tgcccccaaa ttatatttat gtatgtaagt 2100
gaggtttgtt ttgtatatta aaatggagtt tgtttgtaaa aaaaaaaaaa aaaaaaa 2157
<210> SEQ ID NO 82
<211> LENGTH: 1016
<212> TYPE: DNA
<213> ORGANISM: Homo sapiens
<400> SEQUENCE: 82
agcgacctca gaggagtaac cgggccttaa ctttttgcgc tcgttttgct ataatttttc 60
tctatccacc tccatcccac ccccacaaca ctctttactg ggggggtctt ttgtgttccg 120
gatctccccc tccatggctc ccttagccga agtcgggggc tttctgggcg gcctggaggg 180
cttgggccag caggtgggtt cgcatttcct gttgcctcct gccggggagc ggccgccgct 240
gctgggcgag cgcaggagcg cggcggagcg gagcgcgcgc ggcgggccgg gggctgcgca 300
gctggcgcac ctgcacggca tcctgcgccg ccggcagctc tattgccgca ccggcttcca 360
cctgcagatc ctgcccgacg gcagcgtgca gggcacccgg caggaccaca gcctcttcgg 420
tatcttggaa ttcatcagtg tggcagtggg actggtcagt attagaggtg tggacagtgg 480
tctctatctt ggaatgaatg acaaaggaga actctatgga tcagagaaac ttacttccga 540
atgcatcttt agggagcagt ttgaagagaa ctggtataac acctattcat ctaacatata 600
taaacatgga gacactggcc gcaggtattt tgtggcactt aacaaagacg gaactccaag 660
agatggcgcc aggtccaaga ggcatcagaa atttacacat ttcttaccta gaccagtgga 720
tccagaaaga gttccagaat tgtacaagga cctactgatg tacacttgaa gtgcgatagt 780
gacattatgg aagagtcaaa ccacaaccat tctttcttgt catagttccc atcataaaat 840
aatgacccaa ggagacgttc aaaatattaa agtctatttt ctactgagag actggatttg 900
gaaagaatat tgagaaaaaa aaccaaaaaa aattttgact agaaatagat catgatcact 960
ctttatatgt ggattaagtt cccttagata cattggatta gtccttacca gtagac 1016
<210> SEQ ID NO 83
<211> LENGTH: 940
<212> TYPE: DNA
<213> ORGANISM: Homo sapiens
<400> SEQUENCE: 83
ctgtcagctg aggatccagc cgaaagagga gccaggcact caggccacct gagtctactc 60
acctggacaa ctggaatctg gcaccaattc taaaccactc agcttctccg agctcacacc 120
ccggagatca cctgaggacc cgagccattg atggactcgg acgagaccgg gttcgagcac 180
tcaggactgt gggtttctgt gctggctggt cttctgctgg gagcctgcca ggcacacccc 240
atccctgact ccagtcctct cctgcaattc gggggccaag tccggcagcg gtacctctac 300
acagatgatg cccagcagac agaagcccac ctggagatca gggaggatgg gacggtgggg 360
ggcgctgctg accagagccc cgaaagtctc ctgcagctga aagccttgaa gccgggagtt 420
attcaaatct tgggagtcaa gacatccagg ttcctgtgcc agcggccaga tggggccctg 480
tatggatcgc tccactttga ccctgaggcc tgcagcttcc gggagctgct tcttgaggac 540
ggatacaatg tttaccagtc cgaagcccac ggcctcccgc tgcacctgcc agggaacaag 600
tccccacacc gggaccctgc accccgagga ccagctcgct tcctgccact accaggcctg 660
ccccccgcac tcccggagcc acccggaatc ctggcccccc agccccccga tgtgggctcc 720
tcggaccctc tgagcatggt gggaccttcc cagggccgaa gccccagcta cgcttcctga 780
agccagaggc tgtttactat gacatctcct ctttatttat taggttattt atcttattta 840
tttttttatt tttcttactt gagataataa agagttccag aggagaaaaa aaaaaaaaaa 900
aaaaaaaaaa aaaaaaaaaa aaaaaaaaaa aaaaaaaaaa 940
<210> SEQ ID NO 84
<211> LENGTH: 513
<212> TYPE: DNA
<213> ORGANISM: Homo sapiens
<400> SEQUENCE: 84
atgcgccgcc gcctgtggct gggcctggcc tggctgctgc tggcgcgggc gccggacgcc 60
gcgggaaccc cgagcgcgtc gcggggaccg cgcagctacc cgcacctgga gggcgacgtg 120
cgctggcggc gcctcttctc ctccactcac ttcttcctgc gcgtggatcc cggcggccgc 180
gtgcagggca cccgctggcg ccacggccag gacagcatcc tggagatccg ctctgtacac 240
gtgggcgtcg tggtcatcaa agcagtgtcc tcaggcttct acgtggccat gaaccgccgg 300
ggccgcctct acgggtcgcg actctacacc gtggactgca ggttccggga gcgcatcgaa 360
gagaacggcc acaacaccta cgcctcacag cgctggcgcc gccgcggcca gcccatgttc 420
ctggcgctgg acaggagggg ggggccccgg ccaggcggcc ggacgcggcg gtaccacctg 480
tccgcccact tcctgcccgt cctggtctcc tga 513
<210> SEQ ID NO 85
<211> LENGTH: 3018
<212> TYPE: DNA
<213> ORGANISM: Homo sapiens
<400> SEQUENCE: 85
cggcaaaaag gagggaatcc agtctaggat cctcacacca gctacttgca agggagaagg 60
aaaaggccag taaggcctgg gccaggagag tcccgacagg agtgtcaggt ttcaatctca 120
gcaccagcca ctcagagcag ggcacgatgt tgggggcccg cctcaggctc tgggtctgtg 180
ccttgtgcag cgtctgcagc atgagcgtcc tcagagccta tcccaatgcc tccccactgc 240
tcggctccag ctggggtggc ctgatccacc tgtacacagc cacagccagg aacagctacc 300
acctgcagat ccacaagaat ggccatgtgg atggcgcacc ccatcagacc atctacagtg 360
ccctgatgat cagatcagag gatgctggct ttgtggtgat tacaggtgtg atgagcagaa 420
gatacctctg catggatttc agaggcaaca tttttggatc acactatttc gacccggaga 480
actgcaggtt ccaacaccag acgctggaaa acgggtacga cgtctaccac tctcctcagt 540
atcacttcct ggtcagtctg ggccgggcga agagagcctt cctgccaggc atgaacccac 600
ccccgtactc ccagttcctg tcccggagga acgagatccc cctaattcac ttcaacaccc 660
ccataccacg gcggcacacc cggagcgccg aggacgactc ggagcgggac cccctgaacg 720
tgctgaagcc ccgggcccgg atgaccccgg ccccggcctc ctgttcacag gagctcccga 780
gcgccgagga caacagcccg atggccagtg acccattagg ggtggtcagg ggcggtcgag 840
tgaacacgca cgctggggga acgggcccgg aaggctgccg ccccttcgcc aagttcatct 900
agggtcgctg gaagggcacc ctctttaacc catccctcag caaacgcagc tcttcccaag 960
gaccaggtcc cttgacgttc cgaggatggg aaaggtgaca ggggcatgta tggaatttgc 1020
tgcttctctg gggtcccttc cacaggaggt cctgtgagaa ccaacctttg aggcccaagt 1080
catggggttt caccgccttc ctcactccat atagaacacc tttcccaata ggaaacccca 1140
acaggtaaac tagaaatttc cccttcatga aggtagagag aaggggtctc tcccaacata 1200
tttctcttcc ttgtgcctct cctctttatc acttttaagc ataaaaaaaa aaaaaaaaaa 1260
aaaaaaaaaa aaaagcagtg ggttcctgag ctcaagactt tgaaggtgta gggaagagga 1320
aatcggagat cccagaagct tctccactgc cctatgcatt tatgttagat gccccgatcc 1380
cactggcatt tgagtgtgca aaccttgaca ttaacagctg aatggggcaa gttgatgaaa 1440
acactacttt caagccttcg ttcttccttg agcatctctg gggaagagct gtcaaaagac 1500
tggtggtagg ctggtgaaaa cttgacagct agacttgatg cttgctgaaa tgaggcagga 1560
atcataatag aaaactcagc ctccctacag ggtgagcacc ttctgtctcg ctgtctccct 1620
ctgtgcagcc acagccagag ggcccagaat ggccccactc tgttcccaag cagttcatga 1680
tacagcctca ccttttggcc ccatctctgg tttttgaaaa tttggtctaa ggaataaata 1740
gcttttacac tggctcacga aaatctgccc tgctagaatt tgcttttcaa aatggaaata 1800
aattccaact ctcctaagag gcatttaatt aaggctctac ttccaggttg agtaggaatc 1860
cattctgaac aaactacaaa aatgtgactg ggaagggggc tttgagagac tgggactgct 1920
ctgggttagg ttttctgtgg actgaaaaat cgtgtccttt tctctaaatg aagtggcatc 1980
aaggactcag ggggaaagaa atcaggggac atgttataga agttatgaaa agacaaccac 2040
atggtcaggc tcttgtctgt ggtctctagg gctctgcagc agcagtggct cttcgattag 2100
ttaaaactct cctaggctga cacatctggg tctcaatccc cttggaaatt cttggtgcat 2160
taaatgaagc cttaccccat tactgcggtt cttcctgtaa gggggctcca ttttcctccc 2220
tctctttaaa tgaccaccta aaggacagta tattaacaag caaagtcgat tcaacaacag 2280
cttcttccca gtcacttttt tttttctcac tgccatcaca tactaacctt atactttgat 2340
ctattctttt tggttatgag agaaatgttg ggcaactgtt tttacctgat ggttttaagc 2400
tgaacttgaa ggactggttc ctattctgaa acagtaaaac tatgtataat agtatatagc 2460
catgcatggc aaatatttta atatttctgt tttcatttcc tgttggaaat attatcctgc 2520
ataatagcta ttggaggctc ctcagtgaaa gatcccaaaa ggattttggt ggaaaactag 2580
ttgtaatctc acaaactcaa cactaccatc aggggttttc tttatggcaa agccaaaata 2640
gctcctacaa tttcttatat ccctcgtcat gtggcagtat ttatttattt atttggaagt 2700
ttgcctatcc ttctatattt atagatattt ataaaaatgt aacccctttt tcctttcttc 2760
tgtttaaaat aaaaataaaa tttatctcag cttctgttag cttatcctct ttgtagtact 2820
acttaaaagc atgtcggaat ataagaataa aaaggattat gggaggggaa cattagggaa 2880
atccagagaa ggcaaaattg aaaaaaagat tttagaattt taaaattttc aaagatttct 2940
tccattcata aggagactca atgattttaa ttgatctaga cagaattatt taagttttat 3000
caatattgga tttctggt 3018
<210> SEQ ID NO 86
<211> LENGTH: 211
<212> TYPE: PRT
<213> ORGANISM: Homo sapiens
<400> SEQUENCE: 86
Met Arg Thr Leu Ala Cys Leu Leu Leu Leu Gly Cys Gly Tyr Leu Ala
1 5 10 15
His Val Leu Ala Glu Glu Ala Glu Ile Pro Arg Glu Val Ile Glu Arg
                        20 25 30
Leu Ala Arg Ser Gln Ile His Ser Ile Arg Asp Leu Gln Arg Leu Leu
                35 40 45
Glu Ile Asp Ser Val Gly Ser Glu Asp Ser Leu Asp Thr Ser Leu Arg
        50 55 60
Ala His Gly Val His Ala Thr Lys His Val Pro Glu Lys Arg Pro Leu
65 70 75 80
Pro Ile Arg Arg Lys Arg Ser Ile Glu Glu Ala Val Pro Ala Val Cys
                                85 90 95
Lys Thr Arg Thr Val Ile Tyr Glu Ile Pro Arg Ser Gln Val Asp Pro
                        100 105 110
Thr Ser Ala Asn Phe Leu Ile Trp Pro Pro Cys Val Glu Val Lys Arg
                115 120 125
Cys Thr Gly Cys Cys Asn Thr Ser Ser Val Lys Cys Gln Pro Ser Arg
        130 135 140
Val His His Arg Ser Val Lys Val Ala Lys Val Glu Tyr Val Arg Lys
145 150 155 160
Lys Pro Lys Leu Lys Glu Val Gln Val Arg Leu Glu Glu His Leu Glu
                                165 170 175
Cys Ala Cys Ala Thr Thr Ser Leu Asn Pro Asp Tyr Arg Glu Glu Asp
                        180 185 190
Thr Gly Arg Pro Arg Glu Ser Gly Lys Lys Arg Lys Arg Lys Arg Leu
                195 200 205
Lys Pro Thr
        210
<210> SEQ ID NO 87
<211> LENGTH: 196
<212> TYPE: PRT
<213> ORGANISM: Homo sapiens
<400> SEQUENCE: 87
Met Arg Thr Leu Ala Cys Leu Leu Leu Leu Gly Cys Gly Tyr Leu Ala
1 5 10 15
His Val Leu Ala Glu Glu Ala Glu Ile Pro Arg Glu Val Ile Glu Arg
                        20 25 30
Leu Ala Arg Ser Gln Ile His Ser Ile Arg Asp Leu Gln Arg Leu Leu
                35 40 45
Glu Ile Asp Ser Val Gly Ser Glu Asp Ser Leu Asp Thr Ser Leu Arg
        50 55 60
Ala His Gly Val His Ala Thr Lys His Val Pro Glu Lys Arg Pro Leu
65 70 75 80
Pro Ile Arg Arg Lys Arg Ser Ile Glu Glu Ala Val Pro Ala Val Cys
                                85 90 95
Lys Thr Arg Thr Val Ile Tyr Glu Ile Pro Arg Ser Gln Val Asp Pro
                        100 105 110
Thr Ser Ala Asn Phe Leu Ile Trp Pro Pro Cys Val Glu Val Lys Arg
                115 120 125
Cys Thr Gly Cys Cys Asn Thr Ser Ser Val Lys Cys Gln Pro Ser Arg
        130 135 140
Val His His Arg Ser Val Lys Val Ala Lys Val Glu Tyr Val Arg Lys
145 150 155 160
Lys Pro Lys Leu Lys Glu Val Gln Val Arg Leu Glu Glu His Leu Glu
                                165 170 175
Cys Ala Cys Ala Thr Thr Ser Leu Asn Pro Asp Tyr Arg Glu Glu Asp
                        180 185 190
Thr Asp Val Arg
                195
<210> SEQ ID NO 88
<211> LENGTH: 241
<212> TYPE: PRT
<213> ORGANISM: Homo sapiens
<400> SEQUENCE: 88
Met Asn Arg Cys Trp Ala Leu Phe Leu Ser Leu Cys Cys Tyr Leu Arg
1 5 10 15
Leu Val Ser Ala Glu Gly Asp Pro Ile Pro Glu Glu Leu Tyr Glu Met
                        20 25 30
Leu Ser Asp His Ser Ile Arg Ser Phe Asp Asp Leu Gln Arg Leu Leu
                35 40 45
His Gly Asp Pro Gly Glu Glu Asp Gly Ala Glu Leu Asp Leu Asn Met
        50 55 60
Thr Arg Ser His Ser Gly Gly Glu Leu Glu Ser Leu Ala Arg Gly Arg
65 70 75 80
Arg Ser Leu Gly Ser Leu Thr Ile Ala Glu Pro Ala Met Ile Ala Glu
                                85 90 95
Cys Lys Thr Arg Thr Glu Val Phe Glu Ile Ser Arg Arg Leu Ile Asp
                        100 105 110
Arg Thr Asn Ala Asn Phe Leu Val Trp Pro Pro Cys Val Glu Val Gln
                115 120 125
Arg Cys Ser Gly Cys Cys Asn Asn Arg Asn Val Gln Cys Arg Pro Thr
        130 135 140
Gln Val Gln Leu Arg Pro Val Gln Val Arg Lys Ile Glu Ile Val Arg
145 150 155 160
Lys Lys Pro Ile Phe Lys Lys Ala Thr Val Thr Leu Glu Asp His Leu
                                165 170 175
Ala Cys Lys Cys Glu Thr Val Ala Ala Ala Arg Pro Val Thr Arg Ser
                        180 185 190
Pro Gly Gly Ser Gln Glu Gln Arg Ala Lys Thr Pro Gln Thr Arg Val
                195 200 205
Thr Ile Arg Thr Val Arg Val Arg Arg Pro Pro Lys Gly Lys His Arg
        210 215 220
Lys Phe Lys His Thr His Asp Lys Thr Ala Leu Lys Glu Thr Leu Gly
225 230 235 240
Ala
<210> SEQ ID NO 89
<211> LENGTH: 226
<212> TYPE: PRT
<213> ORGANISM: Homo sapiens
<400> SEQUENCE: 89
Met Phe Ile Met Gly Leu Gly Asp Pro Ile Pro Glu Glu Leu Tyr Glu
1 5 10 15
Met Leu Ser Asp His Ser Ile Arg Ser Phe Asp Asp Leu Gln Arg Leu
                        20 25 30
Leu His Gly Asp Pro Gly Glu Glu Asp Gly Ala Glu Leu Asp Leu Asn
                35 40 45
Met Thr Arg Ser His Ser Gly Gly Glu Leu Glu Ser Leu Ala Arg Gly
        50 55 60
Arg Arg Ser Leu Gly Ser Leu Thr Ile Ala Glu Pro Ala Met Ile Ala
65 70 75 80
Glu Cys Lys Thr Arg Thr Glu Val Phe Glu Ile Ser Arg Arg Leu Ile
                                85 90 95
Asp Arg Thr Asn Ala Asn Phe Leu Val Trp Pro Pro Cys Val Glu Val
                        100 105 110
Gln Arg Cys Ser Gly Cys Cys Asn Asn Arg Asn Val Gln Cys Arg Pro
                115 120 125
Thr Gln Val Gln Leu Arg Pro Val Gln Val Arg Lys Ile Glu Ile Val
        130 135 140
Arg Lys Lys Pro Ile Phe Lys Lys Ala Thr Val Thr Leu Glu Asp His
145 150 155 160
Leu Ala Cys Lys Cys Glu Thr Val Ala Ala Ala Arg Pro Val Thr Arg
                                165 170 175
Ser Pro Gly Gly Ser Gln Glu Gln Arg Ala Lys Thr Pro Gln Thr Arg
                        180 185 190
Val Thr Ile Arg Thr Val Arg Val Arg Arg Pro Pro Lys Gly Lys His
                195 200 205
Arg Lys Phe Lys His Thr His Asp Lys Thr Ala Leu Lys Glu Thr Leu
        210 215 220
Gly Ala
225
<210> SEQ ID NO 90
<211> LENGTH: 345
<212> TYPE: PRT
<213> ORGANISM: Homo sapiens
<400> SEQUENCE: 90
Met Ser Leu Phe Gly Leu Leu Leu Leu Thr Ser Ala Leu Ala Gly Gln
1 5 10 15
Arg Gln Gly Thr Gln Ala Glu Ser Asn Leu Ser Ser Lys Phe Gln Phe
                        20 25 30
Ser Ser Asn Lys Glu Gln Asn Gly Val Gln Asp Pro Gln His Glu Arg
                35 40 45
Ile Ile Thr Val Ser Thr Asn Gly Ser Ile His Ser Pro Arg Phe Pro
        50 55 60
His Thr Tyr Pro Arg Asn Thr Val Leu Val Trp Arg Leu Val Ala Val
65 70 75 80
Glu Glu Asn Val Trp Ile Gln Leu Thr Phe Asp Glu Arg Phe Gly Leu
                                85 90 95
Glu Asp Pro Glu Asp Asp Ile Cys Lys Tyr Asp Phe Val Glu Val Glu
                        100 105 110
Glu Pro Ser Asp Gly Thr Ile Leu Gly Arg Trp Cys Gly Ser Gly Thr
                115 120 125
Val Pro Gly Lys Gln Ile Ser Lys Gly Asn Gln Ile Arg Ile Arg Phe
        130 135 140
Val Ser Asp Glu Tyr Phe Pro Ser Glu Pro Gly Phe Cys Ile His Tyr
145 150 155 160
Asn Ile Val Met Pro Gln Phe Thr Glu Ala Val Ser Pro Ser Val Leu
                                165 170 175
Pro Pro Ser Ala Leu Pro Leu Asp Leu Leu Asn Asn Ala Ile Thr Ala
                        180 185 190
Phe Ser Thr Leu Glu Asp Leu Ile Arg Tyr Leu Glu Pro Glu Arg Trp
                195 200 205
Gln Leu Asp Leu Glu Asp Leu Tyr Arg Pro Thr Trp Gln Leu Leu Gly
        210 215 220
Lys Ala Phe Val Phe Gly Arg Lys Ser Arg Val Val Asp Leu Asn Leu
225 230 235 240
Leu Thr Glu Glu Val Arg Leu Tyr Ser Cys Thr Pro Arg Asn Phe Ser
                                245 250 255
Val Ser Ile Arg Glu Glu Leu Lys Arg Thr Asp Thr Ile Phe Trp Pro
                        260 265 270
Gly Cys Leu Leu Val Lys Arg Cys Gly Gly Asn Cys Ala Cys Cys Leu
                275 280 285
His Asn Cys Asn Glu Cys Gln Cys Val Pro Ser Lys Val Thr Lys Lys
        290 295 300
Tyr His Glu Val Leu Gln Leu Arg Pro Lys Thr Gly Val Arg Gly Leu
305 310 315 320
His Lys Ser Leu Thr Asp Val Ala Leu Glu His His Glu Glu Cys Asp
                                325 330 335
Cys Val Cys Arg Gly Ser Thr Gly Gly
                        340 345
<210> SEQ ID NO 91
<211> LENGTH: 370
<212> TYPE: PRT
<213> ORGANISM: Homo sapiens
<400> SEQUENCE: 91
Met His Arg Leu Ile Phe Val Tyr Thr Leu Ile Cys Ala Asn Phe Cys
1 5 10 15
Ser Cys Arg Asp Thr Ser Ala Thr Pro Gln Ser Ala Ser Ile Lys Ala
                        20 25 30
Leu Arg Asn Ala Asn Leu Arg Arg Asp Glu Ser Asn His Leu Thr Asp
                35 40 45
Leu Tyr Arg Arg Asp Glu Thr Ile Gln Val Lys Gly Asn Gly Tyr Val
        50 55 60
Gln Ser Pro Arg Phe Pro Asn Ser Tyr Pro Arg Asn Leu Leu Leu Thr
65 70 75 80
Trp Arg Leu His Ser Gln Glu Asn Thr Arg Ile Gln Leu Val Phe Asp
                                85 90 95
Asn Gln Phe Gly Leu Glu Glu Ala Glu Asn Asp Ile Cys Arg Tyr Asp
                        100 105 110
Phe Val Glu Val Glu Asp Ile Ser Glu Thr Ser Thr Ile Ile Arg Gly
                115 120 125
Arg Trp Cys Gly His Lys Glu Val Pro Pro Arg Ile Lys Ser Arg Thr
        130 135 140
Asn Gln Ile Lys Ile Thr Phe Lys Ser Asp Asp Tyr Phe Val Ala Lys
145 150 155 160
Pro Gly Phe Lys Ile Tyr Tyr Ser Leu Leu Glu Asp Phe Gln Pro Ala
                                165 170 175
Ala Ala Ser Glu Thr Asn Trp Glu Ser Val Thr Ser Ser Ile Ser Gly
                        180 185 190
Val Ser Tyr Asn Ser Pro Ser Val Thr Asp Pro Thr Leu Ile Ala Asp
                195 200 205
Ala Leu Asp Lys Lys Ile Ala Glu Phe Asp Thr Val Glu Asp Leu Leu
        210 215 220
Lys Tyr Phe Asn Pro Glu Ser Trp Gln Glu Asp Leu Glu Asn Met Tyr
225 230 235 240
Leu Asp Thr Pro Arg Tyr Arg Gly Arg Ser Tyr His Asp Arg Lys Ser
                                245 250 255
Lys Val Asp Leu Asp Arg Leu Asn Asp Asp Ala Lys Arg Tyr Ser Cys
                        260 265 270
Thr Pro Arg Asn Tyr Ser Val Asn Ile Arg Glu Glu Leu Lys Leu Ala
                275 280 285
Asn Val Val Phe Phe Pro Arg Cys Leu Leu Val Gln Arg Cys Gly Gly
        290 295 300
Asn Cys Gly Cys Gly Thr Val Asn Trp Arg Ser Cys Thr Cys Asn Ser
305 310 315 320
Gly Lys Thr Val Lys Lys Tyr His Glu Val Leu Gln Phe Glu Pro Gly
                                325 330 335
His Ile Lys Arg Arg Gly Arg Ala Lys Thr Met Ala Leu Val Asp Ile
                        340 345 350
Gln Leu Asp His His Glu Arg Cys Asp Cys Ile Cys Ser Ser Arg Pro
                355 360 365
Pro Arg
        370
<210> SEQ ID NO 92
<211> LENGTH: 364
<212> TYPE: PRT
<213> ORGANISM: Homo sapiens
<400> SEQUENCE: 92
Met His Arg Leu Ile Phe Val Tyr Thr Leu Ile Cys Ala Asn Phe Cys
1 5 10 15
Ser Cys Arg Asp Thr Ser Ala Thr Pro Gln Ser Ala Ser Ile Lys Ala
                        20 25 30
Leu Arg Asn Ala Asn Leu Arg Arg Asp Asp Leu Tyr Arg Arg Asp Glu
                35 40 45
Thr Ile Gln Val Lys Gly Asn Gly Tyr Val Gln Ser Pro Arg Phe Pro
        50 55 60
Asn Ser Tyr Pro Arg Asn Leu Leu Leu Thr Trp Arg Leu His Ser Gln
65 70 75 80
Glu Asn Thr Arg Ile Gln Leu Val Phe Asp Asn Gln Phe Gly Leu Glu
                                85 90 95
Glu Ala Glu Asn Asp Ile Cys Arg Tyr Asp Phe Val Glu Val Glu Asp
                        100 105 110
Ile Ser Glu Thr Ser Thr Ile Ile Arg Gly Arg Trp Cys Gly His Lys
                115 120 125
Glu Val Pro Pro Arg Ile Lys Ser Arg Thr Asn Gln Ile Lys Ile Thr
        130 135 140
Phe Lys Ser Asp Asp Tyr Phe Val Ala Lys Pro Gly Phe Lys Ile Tyr
145 150 155 160
Tyr Ser Leu Leu Glu Asp Phe Gln Pro Ala Ala Ala Ser Glu Thr Asn
                                165 170 175
Trp Glu Ser Val Thr Ser Ser Ile Ser Gly Val Ser Tyr Asn Ser Pro
                        180 185 190
Ser Val Thr Asp Pro Thr Leu Ile Ala Asp Ala Leu Asp Lys Lys Ile
                195 200 205
Ala Glu Phe Asp Thr Val Glu Asp Leu Leu Lys Tyr Phe Asn Pro Glu
        210 215 220
Ser Trp Gln Glu Asp Leu Glu Asn Met Tyr Leu Asp Thr Pro Arg Tyr
225 230 235 240
Arg Gly Arg Ser Tyr His Asp Arg Lys Ser Lys Val Asp Leu Asp Arg
                                245 250 255
Leu Asn Asp Asp Ala Lys Arg Tyr Ser Cys Thr Pro Arg Asn Tyr Ser
                        260 265 270
Val Asn Ile Arg Glu Glu Leu Lys Leu Ala Asn Val Val Phe Phe Pro
                275 280 285
Arg Cys Leu Leu Val Gln Arg Cys Gly Gly Asn Cys Gly Cys Gly Thr
        290 295 300
Val Asn Trp Arg Ser Cys Thr Cys Asn Ser Gly Lys Thr Val Lys Lys
305 310 315 320
Tyr His Glu Val Leu Gln Phe Glu Pro Gly His Ile Lys Arg Arg Gly
                                325 330 335
Arg Ala Lys Thr Met Ala Leu Val Asp Ile Gln Leu Asp His His Glu
                        340 345 350
Arg Cys Asp Cys Ile Cys Ser Ser Arg Pro Pro Arg
                355 360
<210> SEQ ID NO 93
<211> LENGTH: 1207
<212> TYPE: PRT
<213> ORGANISM: Homo sapiens
<400> SEQUENCE: 93
Met Leu Leu Thr Leu Ile Ile Leu Leu Pro Val Val Ser Lys Phe Ser
1 5 10 15
Phe Val Ser Leu Ser Ala Pro Gln His Trp Ser Cys Pro Glu Gly Thr
                        20 25 30
Leu Ala Gly Asn Gly Asn Ser Thr Cys Val Gly Pro Ala Pro Phe Leu
                35 40 45
Ile Phe Ser His Gly Asn Ser Ile Phe Arg Ile Asp Thr Glu Gly Thr
        50 55 60
Asn Tyr Glu Gln Leu Val Val Asp Ala Gly Val Ser Val Ile Met Asp
65 70 75 80
Phe His Tyr Asn Glu Lys Arg Ile Tyr Trp Val Asp Leu Glu Arg Gln
                                85 90 95
Leu Leu Gln Arg Val Phe Leu Asn Gly Ser Arg Gln Glu Arg Val Cys
                        100 105 110
Asn Ile Glu Lys Asn Val Ser Gly Met Ala Ile Asn Trp Ile Asn Glu
                115 120 125
Glu Val Ile Trp Ser Asn Gln Gln Glu Gly Ile Ile Thr Val Thr Asp
        130 135 140
Met Lys Gly Asn Asn Ser His Ile Leu Leu Ser Ala Leu Lys Tyr Pro
145 150 155 160
Ala Asn Val Ala Val Asp Pro Val Glu Arg Phe Ile Phe Trp Ser Ser
                                165 170 175
Glu Val Ala Gly Ser Leu Tyr Arg Ala Asp Leu Asp Gly Val Gly Val
                        180 185 190
Lys Ala Leu Leu Glu Thr Ser Glu Lys Ile Thr Ala Val Ser Leu Asp
                195 200 205
Val Leu Asp Lys Arg Leu Phe Trp Ile Gln Tyr Asn Arg Glu Gly Ser
        210 215 220
Asn Ser Leu Ile Cys Ser Cys Asp Tyr Asp Gly Gly Ser Val His Ile
225 230 235 240
Ser Lys His Pro Thr Gln His Asn Leu Phe Ala Met Ser Leu Phe Gly
                                245 250 255
Asp Arg Ile Phe Tyr Ser Thr Trp Lys Met Lys Thr Ile Trp Ile Ala
                        260 265 270
Asn Lys His Thr Gly Lys Asp Met Val Arg Ile Asn Leu His Ser Ser
                275 280 285
Phe Val Pro Leu Gly Glu Leu Lys Val Val His Pro Leu Ala Gln Pro
        290 295 300
Lys Ala Glu Asp Asp Thr Trp Glu Pro Glu Gln Lys Leu Cys Lys Leu
305 310 315 320
Arg Lys Gly Asn Cys Ser Ser Thr Val Cys Gly Gln Asp Leu Gln Ser
                                325 330 335
His Leu Cys Met Cys Ala Glu Gly Tyr Ala Leu Ser Arg Asp Arg Lys
                        340 345 350
Tyr Cys Glu Asp Val Asn Glu Cys Ala Phe Trp Asn His Gly Cys Thr
                355 360 365
Leu Gly Cys Lys Asn Thr Pro Gly Ser Tyr Tyr Cys Thr Cys Pro Val
        370 375 380
Gly Phe Val Leu Leu Pro Asp Gly Lys Arg Cys His Gln Leu Val Ser
385 390 395 400
Cys Pro Arg Asn Val Ser Glu Cys Ser His Asp Cys Val Leu Thr Ser
                                405 410 415
Glu Gly Pro Leu Cys Phe Cys Pro Glu Gly Ser Val Leu Glu Arg Asp
                        420 425 430
Gly Lys Thr Cys Ser Gly Cys Ser Ser Pro Asp Asn Gly Gly Cys Ser
                435 440 445
Gln Leu Cys Val Pro Leu Ser Pro Val Ser Trp Glu Cys Asp Cys Phe
        450 455 460
Pro Gly Tyr Asp Leu Gln Leu Asp Glu Lys Ser Cys Ala Ala Ser Gly
465 470 475 480
Pro Gln Pro Phe Leu Leu Phe Ala Asn Ser Gln Asp Ile Arg His Met
                                485 490 495
His Phe Asp Gly Thr Asp Tyr Gly Thr Leu Leu Ser Gln Gln Met Gly
                        500 505 510
Met Val Tyr Ala Leu Asp His Asp Pro Val Glu Asn Lys Ile Tyr Phe
                515 520 525
Ala His Thr Ala Leu Lys Trp Ile Glu Arg Ala Asn Met Asp Gly Ser
        530 535 540
Gln Arg Glu Arg Leu Ile Glu Glu Gly Val Asp Val Pro Glu Gly Leu
545 550 555 560
Ala Val Asp Trp Ile Gly Arg Arg Phe Tyr Trp Thr Asp Arg Gly Lys
                                565 570 575
Ser Leu Ile Gly Arg Ser Asp Leu Asn Gly Lys Arg Ser Lys Ile Ile
                        580 585 590
Thr Lys Glu Asn Ile Ser Gln Pro Arg Gly Ile Ala Val His Pro Met
                595 600 605
Ala Lys Arg Leu Phe Trp Thr Asp Thr Gly Ile Asn Pro Arg Ile Glu
        610 615 620
Ser Ser Ser Leu Gln Gly Leu Gly Arg Leu Val Ile Ala Ser Ser Asp
625 630 635 640
Leu Ile Trp Pro Ser Gly Ile Thr Ile Asp Phe Leu Thr Asp Lys Leu
                                645 650 655
Tyr Trp Cys Asp Ala Lys Gln Ser Val Ile Glu Met Ala Asn Leu Asp
                        660 665 670
Gly Ser Lys Arg Arg Arg Leu Thr Gln Asn Asp Val Gly His Pro Phe
                675 680 685
Ala Val Ala Val Phe Glu Asp Tyr Val Trp Phe Ser Asp Trp Ala Met
        690 695 700
Pro Ser Val Met Arg Val Asn Lys Arg Thr Gly Lys Asp Arg Val Arg
705 710 715 720
Leu Gln Gly Ser Met Leu Lys Pro Ser Ser Leu Val Val Val His Pro
                                725 730 735
Leu Ala Lys Pro Gly Ala Asp Pro Cys Leu Tyr Gln Asn Gly Gly Cys
                        740 745 750
Glu His Ile Cys Lys Lys Arg Leu Gly Thr Ala Trp Cys Ser Cys Arg
                755 760 765
Glu Gly Phe Met Lys Ala Ser Asp Gly Lys Thr Cys Leu Ala Leu Asp
        770 775 780
Gly His Gln Leu Leu Ala Gly Gly Glu Val Asp Leu Lys Asn Gln Val
785 790 795 800
Thr Pro Leu Asp Ile Leu Ser Lys Thr Arg Val Ser Glu Asp Asn Ile
                                805 810 815
Thr Glu Ser Gln His Met Leu Val Ala Glu Ile Met Val Ser Asp Gln
                        820 825 830
Asp Asp Cys Ala Pro Val Gly Cys Ser Met Tyr Ala Arg Cys Ile Ser
                835 840 845
Glu Gly Glu Asp Ala Thr Cys Gln Cys Leu Lys Gly Phe Ala Gly Asp
        850 855 860
Gly Lys Leu Cys Ser Asp Ile Asp Glu Cys Glu Met Gly Val Pro Val
865 870 875 880
Cys Pro Pro Ala Ser Ser Lys Cys Ile Asn Thr Glu Gly Gly Tyr Val
                                885 890 895
Cys Arg Cys Ser Glu Gly Tyr Gln Gly Asp Gly Ile His Cys Leu Asp
                        900 905 910
Ile Asp Glu Cys Gln Leu Gly Glu His Ser Cys Gly Glu Asn Ala Ser
                915 920 925
Cys Thr Asn Thr Glu Gly Gly Tyr Thr Cys Met Cys Ala Gly Arg Leu
        930 935 940
Ser Glu Pro Gly Leu Ile Cys Pro Asp Ser Thr Pro Pro Pro His Leu
945 950 955 960
Arg Glu Asp Asp His His Tyr Ser Val Arg Asn Ser Asp Ser Glu Cys
                                965 970 975
Pro Leu Ser His Asp Gly Tyr Cys Leu His Asp Gly Val Cys Met Tyr
                        980 985 990
Ile Glu Ala Leu Asp Lys Tyr Ala Cys Asn Cys Val Val Gly Tyr Ile
                995 1000 1005
Gly Glu Arg Cys Gln Tyr Arg Asp Leu Lys Trp Trp Glu Leu Arg
        1010 1015 1020
His Ala Gly His Gly Gln Gln Gln Lys Val Ile Val Val Ala Val
        1025 1030 1035
Cys Val Val Val Leu Val Met Leu Leu Leu Leu Ser Leu Trp Gly
        1040 1045 1050
Ala His Tyr Tyr Arg Thr Gln Lys Leu Leu Ser Lys Asn Pro Lys
        1055 1060 1065
Asn Pro Tyr Glu Glu Ser Ser Arg Asp Val Arg Ser Arg Arg Pro
        1070 1075 1080
Ala Asp Thr Glu Asp Gly Met Ser Ser Cys Pro Gln Pro Trp Phe
        1085 1090 1095
Val Val Ile Lys Glu His Gln Asp Leu Lys Asn Gly Gly Gln Pro
        1100 1105 1110
Val Ala Gly Glu Asp Gly Gln Ala Ala Asp Gly Ser Met Gln Pro
        1115 1120 1125
Thr Ser Trp Arg Gln Glu Pro Gln Leu Cys Gly Met Gly Thr Glu
        1130 1135 1140
Gln Gly Cys Trp Ile Pro Val Ser Ser Asp Lys Gly Ser Cys Pro
        1145 1150 1155
Gln Val Met Glu Arg Ser Phe His Met Pro Ser Tyr Gly Thr Gln
        1160 1165 1170
Thr Leu Glu Gly Gly Val Glu Lys Pro His Ser Leu Leu Ser Ala
        1175 1180 1185
Asn Pro Leu Trp Gln Gln Arg Ala Leu Asp Pro Pro His Gln Met
        1190 1195 1200
Glu Leu Thr Gln
        1205
<210> SEQ ID NO 94
<211> LENGTH: 1166
<212> TYPE: PRT
<213> ORGANISM: Homo sapiens
<400> SEQUENCE: 94
Met Leu Leu Thr Leu Ile Ile Leu Leu Pro Val Val Ser Lys Phe Ser
1 5 10 15
Phe Val Ser Leu Ser Ala Pro Gln His Trp Ser Cys Pro Glu Gly Thr
                        20 25 30
Leu Ala Gly Asn Gly Asn Ser Thr Cys Val Gly Pro Ala Pro Phe Leu
                35 40 45
Ile Phe Ser His Gly Asn Ser Ile Phe Arg Ile Asp Thr Glu Gly Thr
        50 55 60
Asn Tyr Glu Gln Leu Val Val Asp Ala Gly Val Ser Val Ile Met Asp
65 70 75 80
Phe His Tyr Asn Glu Lys Arg Ile Tyr Trp Val Asp Leu Glu Arg Gln
                                85 90 95
Leu Leu Gln Arg Val Phe Leu Asn Gly Ser Arg Gln Glu Arg Val Cys
                        100 105 110
Asn Ile Glu Lys Asn Val Ser Gly Met Ala Ile Asn Trp Ile Asn Glu
                115 120 125
Glu Val Ile Trp Ser Asn Gln Gln Glu Gly Ile Ile Thr Val Thr Asp
        130 135 140
Met Lys Gly Asn Asn Ser His Ile Leu Leu Ser Ala Leu Lys Tyr Pro
145 150 155 160
Ala Asn Val Ala Val Asp Pro Val Glu Arg Phe Ile Phe Trp Ser Ser
                                165 170 175
Glu Val Ala Gly Ser Leu Tyr Arg Ala Asp Leu Asp Gly Val Gly Val
                        180 185 190
Lys Ala Leu Leu Glu Thr Ser Glu Lys Ile Thr Ala Val Ser Leu Asp
                195 200 205
Val Leu Asp Lys Arg Leu Phe Trp Ile Gln Tyr Asn Arg Glu Gly Ser
        210 215 220
Asn Ser Leu Ile Cys Ser Cys Asp Tyr Asp Gly Gly Ser Val His Ile
225 230 235 240
Ser Lys His Pro Thr Gln His Asn Leu Phe Ala Met Ser Leu Phe Gly
                                245 250 255
Asp Arg Ile Phe Tyr Ser Thr Trp Lys Met Lys Thr Ile Trp Ile Ala
                        260 265 270
Asn Lys His Thr Gly Lys Asp Met Val Arg Ile Asn Leu His Ser Ser
                275 280 285
Phe Val Pro Leu Gly Glu Leu Lys Val Val His Pro Leu Ala Gln Pro
        290 295 300
Lys Ala Glu Asp Asp Thr Trp Glu Pro Glu Gln Lys Leu Cys Lys Leu
305 310 315 320
Arg Lys Gly Asn Cys Ser Ser Thr Val Cys Gly Gln Asp Leu Gln Ser
                                325 330 335
His Leu Cys Met Cys Ala Glu Gly Tyr Ala Leu Ser Arg Asp Arg Lys
                        340 345 350
Tyr Cys Glu Asp Val Asn Glu Cys Ala Phe Trp Asn His Gly Cys Thr
                355 360 365
Leu Gly Cys Lys Asn Thr Pro Gly Ser Tyr Tyr Cys Thr Cys Pro Val
        370 375 380
Gly Phe Val Leu Leu Pro Asp Gly Lys Arg Cys His Gln Leu Val Ser
385 390 395 400
Cys Pro Arg Asn Val Ser Glu Cys Ser His Asp Cys Val Leu Thr Ser
                                405 410 415
Glu Gly Pro Leu Cys Phe Cys Pro Glu Gly Ser Val Leu Glu Arg Asp
                        420 425 430
Gly Lys Thr Cys Ser Gly Cys Ser Ser Pro Asp Asn Gly Gly Cys Ser
                435 440 445
Gln Leu Cys Val Pro Leu Ser Pro Val Ser Trp Glu Cys Asp Cys Phe
        450 455 460
Pro Gly Tyr Asp Leu Gln Leu Asp Glu Lys Ser Cys Ala Ala Ser Gly
465 470 475 480
Pro Gln Pro Phe Leu Leu Phe Ala Asn Ser Gln Asp Ile Arg His Met
                                485 490 495
His Phe Asp Gly Thr Asp Tyr Gly Thr Leu Leu Ser Gln Gln Met Gly
                        500 505 510
Met Val Tyr Ala Leu Asp His Asp Pro Val Glu Asn Lys Ile Tyr Phe
                515 520 525
Ala His Thr Ala Leu Lys Trp Ile Glu Arg Ala Asn Met Asp Gly Ser
        530 535 540
Gln Arg Glu Arg Leu Ile Glu Glu Gly Val Asp Val Pro Glu Gly Leu
545 550 555 560
Ala Val Asp Trp Ile Gly Arg Arg Phe Tyr Trp Thr Asp Arg Gly Lys
                                565 570 575
Ser Leu Ile Gly Arg Ser Asp Leu Asn Gly Lys Arg Ser Lys Ile Ile
                        580 585 590
Thr Lys Glu Asn Ile Ser Gln Pro Arg Gly Ile Ala Val His Pro Met
                595 600 605
Ala Lys Arg Leu Phe Trp Thr Asp Thr Gly Ile Asn Pro Arg Ile Glu
        610 615 620
Ser Ser Ser Leu Gln Gly Leu Gly Arg Leu Val Ile Ala Ser Ser Asp
625 630 635 640
Leu Ile Trp Pro Ser Gly Ile Thr Ile Asp Phe Leu Thr Asp Lys Leu
                                645 650 655
Tyr Trp Cys Asp Ala Lys Gln Ser Val Ile Glu Met Ala Asn Leu Asp
                        660 665 670
Gly Ser Lys Arg Arg Arg Leu Thr Gln Asn Asp Val Gly His Pro Phe
                675 680 685
Ala Val Ala Val Phe Glu Asp Tyr Val Trp Phe Ser Asp Trp Ala Met
        690 695 700
Pro Ser Val Met Arg Val Asn Lys Arg Thr Gly Lys Asp Arg Val Arg
705 710 715 720
Leu Gln Gly Ser Met Leu Lys Pro Ser Ser Leu Val Val Val His Pro
                                725 730 735
Leu Ala Lys Pro Gly Ala Asp Pro Cys Leu Tyr Gln Asn Gly Gly Cys
                        740 745 750
Glu His Ile Cys Lys Lys Arg Leu Gly Thr Ala Trp Cys Ser Cys Arg
                755 760 765
Glu Gly Phe Met Lys Ala Ser Asp Gly Lys Thr Cys Leu Ala Leu Asp
        770 775 780
Gly His Gln Leu Leu Ala Gly Gly Glu Val Asp Leu Lys Asn Gln Val
785 790 795 800
Thr Pro Leu Asp Ile Leu Ser Lys Thr Arg Val Ser Glu Asp Asn Ile
                                805 810 815
Thr Glu Ser Gln His Met Leu Val Ala Glu Ile Met Val Ser Asp Gln
                        820 825 830
Asp Asp Cys Ala Pro Val Gly Cys Ser Met Tyr Ala Arg Cys Ile Ser
                835 840 845
Glu Gly Glu Asp Ala Thr Cys Gln Cys Leu Lys Gly Phe Ala Gly Asp
        850 855 860
Gly Lys Leu Cys Ser Asp Ile Asp Glu Cys Glu Met Gly Val Pro Val
865 870 875 880
Cys Pro Pro Ala Ser Ser Lys Cys Ile Asn Thr Glu Gly Gly Tyr Val
                                885 890 895
Cys Arg Cys Ser Glu Gly Tyr Gln Gly Asp Gly Ile His Cys Leu Asp
                        900 905 910
Ser Thr Pro Pro Pro His Leu Arg Glu Asp Asp His His Tyr Ser Val
                915 920 925
Arg Asn Ser Asp Ser Glu Cys Pro Leu Ser His Asp Gly Tyr Cys Leu
        930 935 940
His Asp Gly Val Cys Met Tyr Ile Glu Ala Leu Asp Lys Tyr Ala Cys
945 950 955 960
Asn Cys Val Val Gly Tyr Ile Gly Glu Arg Cys Gln Tyr Arg Asp Leu
                                965 970 975
Lys Trp Trp Glu Leu Arg His Ala Gly His Gly Gln Gln Gln Lys Val
                        980 985 990
Ile Val Val Ala Val Cys Val Val Val Leu Val Met Leu Leu Leu Leu
                995 1000 1005
Ser Leu Trp Gly Ala His Tyr Tyr Arg Thr Gln Lys Leu Leu Ser
        1010 1015 1020
Lys Asn Pro Lys Asn Pro Tyr Glu Glu Ser Ser Arg Asp Val Arg
        1025 1030 1035
Ser Arg Arg Pro Ala Asp Thr Glu Asp Gly Met Ser Ser Cys Pro
        1040 1045 1050
Gln Pro Trp Phe Val Val Ile Lys Glu His Gln Asp Leu Lys Asn
        1055 1060 1065
Gly Gly Gln Pro Val Ala Gly Glu Asp Gly Gln Ala Ala Asp Gly
        1070 1075 1080
Ser Met Gln Pro Thr Ser Trp Arg Gln Glu Pro Gln Leu Cys Gly
        1085 1090 1095
Met Gly Thr Glu Gln Gly Cys Trp Ile Pro Val Ser Ser Asp Lys
        1100 1105 1110
Gly Ser Cys Pro Gln Val Met Glu Arg Ser Phe His Met Pro Ser
        1115 1120 1125
Tyr Gly Thr Gln Thr Leu Glu Gly Gly Val Glu Lys Pro His Ser
        1130 1135 1140
Leu Leu Ser Ala Asn Pro Leu Trp Gln Gln Arg Ala Leu Asp Pro
        1145 1150 1155
Pro His Gln Met Glu Leu Thr Gln
        1160 1165
<210> SEQ ID NO 95
<211> LENGTH: 1165
<212> TYPE: PRT
<213> ORGANISM: Homo sapiens
<400> SEQUENCE: 95
Met Leu Leu Thr Leu Ile Ile Leu Leu Pro Val Val Ser Lys Phe Ser
1 5 10 15
Phe Val Ser Leu Ser Ala Pro Gln His Trp Ser Cys Pro Glu Gly Thr
                        20 25 30
Leu Ala Gly Asn Gly Asn Ser Thr Cys Val Gly Pro Ala Pro Phe Leu
                35 40 45
Ile Phe Ser His Gly Asn Ser Ile Phe Arg Ile Asp Thr Glu Gly Thr
        50 55 60
Asn Tyr Glu Gln Leu Val Val Asp Ala Gly Val Ser Val Ile Met Asp
65 70 75 80
Phe His Tyr Asn Glu Lys Arg Ile Tyr Trp Val Asp Leu Glu Arg Gln
                                85 90 95
Leu Leu Gln Arg Val Phe Leu Asn Gly Ser Arg Gln Glu Arg Val Cys
                        100 105 110
Asn Ile Glu Lys Asn Val Ser Gly Met Ala Ile Asn Trp Ile Asn Glu
                115 120 125
Glu Val Ile Trp Ser Asn Gln Gln Glu Gly Ile Ile Thr Val Thr Asp
        130 135 140
Met Lys Gly Asn Asn Ser His Ile Leu Leu Ser Ala Leu Lys Tyr Pro
145 150 155 160
Ala Asn Val Ala Val Asp Pro Val Glu Arg Phe Ile Phe Trp Ser Ser
                                165 170 175
Glu Val Ala Gly Ser Leu Tyr Arg Ala Asp Leu Asp Gly Val Gly Val
                        180 185 190
Lys Ala Leu Leu Glu Thr Ser Glu Lys Ile Thr Ala Val Ser Leu Asp
                195 200 205
Val Leu Asp Lys Arg Leu Phe Trp Ile Gln Tyr Asn Arg Glu Gly Ser
        210 215 220
Asn Ser Leu Ile Cys Ser Cys Asp Tyr Asp Gly Gly Ser Val His Ile
225 230 235 240
Ser Lys His Pro Thr Gln His Asn Leu Phe Ala Met Ser Leu Phe Gly
                                245 250 255
Asp Arg Ile Phe Tyr Ser Thr Trp Lys Met Lys Thr Ile Trp Ile Ala
                        260 265 270
Asn Lys His Thr Gly Lys Asp Met Val Arg Ile Asn Leu His Ser Ser
                275 280 285
Phe Val Pro Leu Gly Glu Leu Lys Val Val His Pro Leu Ala Gln Pro
        290 295 300
Lys Ala Glu Asp Asp Thr Trp Glu Pro Asp Val Asn Glu Cys Ala Phe
305 310 315 320
Trp Asn His Gly Cys Thr Leu Gly Cys Lys Asn Thr Pro Gly Ser Tyr
                                325 330 335
Tyr Cys Thr Cys Pro Val Gly Phe Val Leu Leu Pro Asp Gly Lys Arg
                        340 345 350
Cys His Gln Leu Val Ser Cys Pro Arg Asn Val Ser Glu Cys Ser His
                355 360 365
Asp Cys Val Leu Thr Ser Glu Gly Pro Leu Cys Phe Cys Pro Glu Gly
        370 375 380
Ser Val Leu Glu Arg Asp Gly Lys Thr Cys Ser Gly Cys Ser Ser Pro
385 390 395 400
Asp Asn Gly Gly Cys Ser Gln Leu Cys Val Pro Leu Ser Pro Val Ser
                                405 410 415
Trp Glu Cys Asp Cys Phe Pro Gly Tyr Asp Leu Gln Leu Asp Glu Lys
                        420 425 430
Ser Cys Ala Ala Ser Gly Pro Gln Pro Phe Leu Leu Phe Ala Asn Ser
                435 440 445
Gln Asp Ile Arg His Met His Phe Asp Gly Thr Asp Tyr Gly Thr Leu
        450 455 460
Leu Ser Gln Gln Met Gly Met Val Tyr Ala Leu Asp His Asp Pro Val
465 470 475 480
Glu Asn Lys Ile Tyr Phe Ala His Thr Ala Leu Lys Trp Ile Glu Arg
                                485 490 495
Ala Asn Met Asp Gly Ser Gln Arg Glu Arg Leu Ile Glu Glu Gly Val
                        500 505 510
Asp Val Pro Glu Gly Leu Ala Val Asp Trp Ile Gly Arg Arg Phe Tyr
                515 520 525
Trp Thr Asp Arg Gly Lys Ser Leu Ile Gly Arg Ser Asp Leu Asn Gly
        530 535 540
Lys Arg Ser Lys Ile Ile Thr Lys Glu Asn Ile Ser Gln Pro Arg Gly
545 550 555 560
Ile Ala Val His Pro Met Ala Lys Arg Leu Phe Trp Thr Asp Thr Gly
                                565 570 575
Ile Asn Pro Arg Ile Glu Ser Ser Ser Leu Gln Gly Leu Gly Arg Leu
                        580 585 590
Val Ile Ala Ser Ser Asp Leu Ile Trp Pro Ser Gly Ile Thr Ile Asp
                595 600 605
Phe Leu Thr Asp Lys Leu Tyr Trp Cys Asp Ala Lys Gln Ser Val Ile
        610 615 620
Glu Met Ala Asn Leu Asp Gly Ser Lys Arg Arg Arg Leu Thr Gln Asn
625 630 635 640
Asp Val Gly His Pro Phe Ala Val Ala Val Phe Glu Asp Tyr Val Trp
                                645 650 655
Phe Ser Asp Trp Ala Met Pro Ser Val Met Arg Val Asn Lys Arg Thr
                        660 665 670
Gly Lys Asp Arg Val Arg Leu Gln Gly Ser Met Leu Lys Pro Ser Ser
                675 680 685
Leu Val Val Val His Pro Leu Ala Lys Pro Gly Ala Asp Pro Cys Leu
        690 695 700
Tyr Gln Asn Gly Gly Cys Glu His Ile Cys Lys Lys Arg Leu Gly Thr
705 710 715 720
Ala Trp Cys Ser Cys Arg Glu Gly Phe Met Lys Ala Ser Asp Gly Lys
                                725 730 735
Thr Cys Leu Ala Leu Asp Gly His Gln Leu Leu Ala Gly Gly Glu Val
                        740 745 750
Asp Leu Lys Asn Gln Val Thr Pro Leu Asp Ile Leu Ser Lys Thr Arg
                755 760 765
Val Ser Glu Asp Asn Ile Thr Glu Ser Gln His Met Leu Val Ala Glu
        770 775 780
Ile Met Val Ser Asp Gln Asp Asp Cys Ala Pro Val Gly Cys Ser Met
785 790 795 800
Tyr Ala Arg Cys Ile Ser Glu Gly Glu Asp Ala Thr Cys Gln Cys Leu
                                805 810 815
Lys Gly Phe Ala Gly Asp Gly Lys Leu Cys Ser Asp Ile Asp Glu Cys
                        820 825 830
Glu Met Gly Val Pro Val Cys Pro Pro Ala Ser Ser Lys Cys Ile Asn
                835 840 845
Thr Glu Gly Gly Tyr Val Cys Arg Cys Ser Glu Gly Tyr Gln Gly Asp
        850 855 860
Gly Ile His Cys Leu Asp Ile Asp Glu Cys Gln Leu Gly Glu His Ser
865 870 875 880
Cys Gly Glu Asn Ala Ser Cys Thr Asn Thr Glu Gly Gly Tyr Thr Cys
                                885 890 895
Met Cys Ala Gly Arg Leu Ser Glu Pro Gly Leu Ile Cys Pro Asp Ser
                        900 905 910
Thr Pro Pro Pro His Leu Arg Glu Asp Asp His His Tyr Ser Val Arg
                915 920 925
Asn Ser Asp Ser Glu Cys Pro Leu Ser His Asp Gly Tyr Cys Leu His
        930 935 940
Asp Gly Val Cys Met Tyr Ile Glu Ala Leu Asp Lys Tyr Ala Cys Asn
945 950 955 960
Cys Val Val Gly Tyr Ile Gly Glu Arg Cys Gln Tyr Arg Asp Leu Lys
                                965 970 975
Trp Trp Glu Leu Arg His Ala Gly His Gly Gln Gln Gln Lys Val Ile
                        980 985 990
Val Val Ala Val Cys Val Val Val Leu Val Met Leu Leu Leu Leu Ser
                995 1000 1005
Leu Trp Gly Ala His Tyr Tyr Arg Thr Gln Lys Leu Leu Ser Lys
        1010 1015 1020
Asn Pro Lys Asn Pro Tyr Glu Glu Ser Ser Arg Asp Val Arg Ser
        1025 1030 1035
Arg Arg Pro Ala Asp Thr Glu Asp Gly Met Ser Ser Cys Pro Gln
        1040 1045 1050
Pro Trp Phe Val Val Ile Lys Glu His Gln Asp Leu Lys Asn Gly
        1055 1060 1065
Gly Gln Pro Val Ala Gly Glu Asp Gly Gln Ala Ala Asp Gly Ser
        1070 1075 1080
Met Gln Pro Thr Ser Trp Arg Gln Glu Pro Gln Leu Cys Gly Met
        1085 1090 1095
Gly Thr Glu Gln Gly Cys Trp Ile Pro Val Ser Ser Asp Lys Gly
        1100 1105 1110
Ser Cys Pro Gln Val Met Glu Arg Ser Phe His Met Pro Ser Tyr
        1115 1120 1125
Gly Thr Gln Thr Leu Glu Gly Gly Val Glu Lys Pro His Ser Leu
        1130 1135 1140
Leu Ser Ala Asn Pro Leu Trp Gln Gln Arg Ala Leu Asp Pro Pro
        1145 1150 1155
His Gln Met Glu Leu Thr Gln
        1160 1165
<210> SEQ ID NO 96
<211> LENGTH: 232
<212> TYPE: PRT
<213> ORGANISM: Homo sapiens
<400> SEQUENCE: 96
Met Asn Phe Leu Leu Ser Trp Val His Trp Ser Leu Ala Leu Leu Leu
1 5 10 15
Tyr Leu His His Ala Lys Trp Ser Gln Ala Ala Pro Met Ala Glu Gly
                        20 25 30
Gly Gly Gln Asn His His Glu Val Val Lys Phe Met Asp Val Tyr Gln
                35 40 45
Arg Ser Tyr Cys His Pro Ile Glu Thr Leu Val Asp Ile Phe Gln Glu
        50 55 60
Tyr Pro Asp Glu Ile Glu Tyr Ile Phe Lys Pro Ser Cys Val Pro Leu
65 70 75 80
Met Arg Cys Gly Gly Cys Cys Asn Asp Glu Gly Leu Glu Cys Val Pro
                                85 90 95
Thr Glu Glu Ser Asn Ile Thr Met Gln Ile Met Arg Ile Lys Pro His
                        100 105 110
Gln Gly Gln His Ile Gly Glu Met Ser Phe Leu Gln His Asn Lys Cys
                115 120 125
Glu Cys Arg Pro Lys Lys Asp Arg Ala Arg Gln Glu Lys Lys Ser Val
        130 135 140
Arg Gly Lys Gly Lys Gly Gln Lys Arg Lys Arg Lys Lys Ser Arg Tyr
145 150 155 160
Lys Ser Trp Ser Val Tyr Val Gly Ala Arg Cys Cys Leu Met Pro Trp
                                165 170 175
Ser Leu Pro Gly Pro His Pro Cys Gly Pro Cys Ser Glu Arg Arg Lys
                        180 185 190
His Leu Phe Val Gln Asp Pro Gln Thr Cys Lys Cys Ser Cys Lys Asn
                195 200 205
Thr Asp Ser Arg Cys Lys Ala Arg Gln Leu Glu Leu Asn Glu Arg Thr
        210 215 220
Cys Arg Cys Asp Lys Pro Arg Arg
225 230
<210> SEQ ID NO 97
<211> LENGTH: 412
<212> TYPE: PRT
<213> ORGANISM: Homo sapiens
<400> SEQUENCE: 97
Met Thr Asp Arg Gln Thr Asp Thr Ala Pro Ser Pro Ser Tyr His Leu
1 5 10 15
Leu Pro Gly Arg Arg Arg Thr Val Asp Ala Ala Ala Ser Arg Gly Gln
                        20 25 30
Gly Pro Glu Pro Ala Pro Gly Gly Gly Val Glu Gly Val Gly Ala Arg
                35 40 45
Gly Val Ala Leu Lys Leu Phe Val Gln Leu Leu Gly Cys Ser Arg Phe
        50 55 60
Gly Gly Ala Val Val Arg Ala Gly Glu Ala Glu Pro Ser Gly Ala Ala
65 70 75 80
Arg Ser Ala Ser Ser Gly Arg Glu Glu Pro Gln Pro Glu Glu Gly Glu
                                85 90 95
Glu Glu Glu Glu Lys Glu Glu Glu Arg Gly Pro Gln Trp Arg Leu Gly
                        100 105 110
Ala Arg Lys Pro Gly Ser Trp Thr Gly Glu Ala Ala Val Cys Ala Asp
                115 120 125
Ser Ala Pro Ala Ala Arg Ala Pro Gln Ala Leu Ala Arg Ala Ser Gly
        130 135 140
Arg Gly Gly Arg Val Ala Arg Arg Gly Ala Glu Glu Ser Gly Pro Pro
145 150 155 160
His Ser Pro Ser Arg Arg Gly Ser Ala Ser Arg Ala Gly Pro Gly Arg
                                165 170 175
Ala Ser Glu Thr Met Asn Phe Leu Leu Ser Trp Val His Trp Ser Leu
                        180 185 190
Ala Leu Leu Leu Tyr Leu His His Ala Lys Trp Ser Gln Ala Ala Pro
                195 200 205
Met Ala Glu Gly Gly Gly Gln Asn His His Glu Val Val Lys Phe Met
        210 215 220
Asp Val Tyr Gln Arg Ser Tyr Cys His Pro Ile Glu Thr Leu Val Asp
225 230 235 240
Ile Phe Gln Glu Tyr Pro Asp Glu Ile Glu Tyr Ile Phe Lys Pro Ser
                                245 250 255
Cys Val Pro Leu Met Arg Cys Gly Gly Cys Cys Asn Asp Glu Gly Leu
                        260 265 270
Glu Cys Val Pro Thr Glu Glu Ser Asn Ile Thr Met Gln Ile Met Arg
                275 280 285
Ile Lys Pro His Gln Gly Gln His Ile Gly Glu Met Ser Phe Leu Gln
        290 295 300
His Asn Lys Cys Glu Cys Arg Pro Lys Lys Asp Arg Ala Arg Gln Glu
305 310 315 320
Lys Lys Ser Val Arg Gly Lys Gly Lys Gly Gln Lys Arg Lys Arg Lys
                                325 330 335
Lys Ser Arg Tyr Lys Ser Trp Ser Val Tyr Val Gly Ala Arg Cys Cys
                        340 345 350
Leu Met Pro Trp Ser Leu Pro Gly Pro His Pro Cys Gly Pro Cys Ser
                355 360 365
Glu Arg Arg Lys His Leu Phe Val Gln Asp Pro Gln Thr Cys Lys Cys
        370 375 380
Ser Cys Lys Asn Thr Asp Ser Arg Cys Lys Ala Arg Gln Leu Glu Leu
385 390 395 400
Asn Glu Arg Thr Cys Arg Cys Asp Lys Pro Arg Arg
                                405 410
<210> SEQ ID NO 98
<211> LENGTH: 215
<212> TYPE: PRT
<213> ORGANISM: Homo sapiens
<400> SEQUENCE: 98
Met Asn Phe Leu Leu Ser Trp Val His Trp Ser Leu Ala Leu Leu Leu
1 5 10 15
Tyr Leu His His Ala Lys Trp Ser Gln Ala Ala Pro Met Ala Glu Gly
                        20 25 30
Gly Gly Gln Asn His His Glu Val Val Lys Phe Met Asp Val Tyr Gln
                35 40 45
Arg Ser Tyr Cys His Pro Ile Glu Thr Leu Val Asp Ile Phe Gln Glu
        50 55 60
Tyr Pro Asp Glu Ile Glu Tyr Ile Phe Lys Pro Ser Cys Val Pro Leu
65 70 75 80
Met Arg Cys Gly Gly Cys Cys Asn Asp Glu Gly Leu Glu Cys Val Pro
                                85 90 95
Thr Glu Glu Ser Asn Ile Thr Met Gln Ile Met Arg Ile Lys Pro His
                        100 105 110
Gln Gly Gln His Ile Gly Glu Met Ser Phe Leu Gln His Asn Lys Cys
                115 120 125
Glu Cys Arg Pro Lys Lys Asp Arg Ala Arg Gln Glu Lys Lys Ser Val
        130 135 140
Arg Gly Lys Gly Lys Gly Gln Lys Arg Lys Arg Lys Lys Ser Arg Tyr
145 150 155 160
Lys Ser Trp Ser Val Pro Cys Gly Pro Cys Ser Glu Arg Arg Lys His
                                165 170 175
Leu Phe Val Gln Asp Pro Gln Thr Cys Lys Cys Ser Cys Lys Asn Thr
                        180 185 190
Asp Ser Arg Cys Lys Ala Arg Gln Leu Glu Leu Asn Glu Arg Thr Cys
                195 200 205
Arg Cys Asp Lys Pro Arg Arg
        210 215
<210> SEQ ID NO 99
<211> LENGTH: 395
<212> TYPE: PRT
<213> ORGANISM: Homo sapiens
<400> SEQUENCE: 99
Met Thr Asp Arg Gln Thr Asp Thr Ala Pro Ser Pro Ser Tyr His Leu
1 5 10 15
Leu Pro Gly Arg Arg Arg Thr Val Asp Ala Ala Ala Ser Arg Gly Gln
                        20 25 30
Gly Pro Glu Pro Ala Pro Gly Gly Gly Val Glu Gly Val Gly Ala Arg
                35 40 45
Gly Val Ala Leu Lys Leu Phe Val Gln Leu Leu Gly Cys Ser Arg Phe
        50 55 60
Gly Gly Ala Val Val Arg Ala Gly Glu Ala Glu Pro Ser Gly Ala Ala
65 70 75 80
Arg Ser Ala Ser Ser Gly Arg Glu Glu Pro Gln Pro Glu Glu Gly Glu
                                85 90 95
Glu Glu Glu Glu Lys Glu Glu Glu Arg Gly Pro Gln Trp Arg Leu Gly
                        100 105 110
Ala Arg Lys Pro Gly Ser Trp Thr Gly Glu Ala Ala Val Cys Ala Asp
                115 120 125
Ser Ala Pro Ala Ala Arg Ala Pro Gln Ala Leu Ala Arg Ala Ser Gly
        130 135 140
Arg Gly Gly Arg Val Ala Arg Arg Gly Ala Glu Glu Ser Gly Pro Pro
145 150 155 160
His Ser Pro Ser Arg Arg Gly Ser Ala Ser Arg Ala Gly Pro Gly Arg
                                165 170 175
Ala Ser Glu Thr Met Asn Phe Leu Leu Ser Trp Val His Trp Ser Leu
                        180 185 190
Ala Leu Leu Leu Tyr Leu His His Ala Lys Trp Ser Gln Ala Ala Pro
                195 200 205
Met Ala Glu Gly Gly Gly Gln Asn His His Glu Val Val Lys Phe Met
        210 215 220
Asp Val Tyr Gln Arg Ser Tyr Cys His Pro Ile Glu Thr Leu Val Asp
225 230 235 240
Ile Phe Gln Glu Tyr Pro Asp Glu Ile Glu Tyr Ile Phe Lys Pro Ser
                                245 250 255
Cys Val Pro Leu Met Arg Cys Gly Gly Cys Cys Asn Asp Glu Gly Leu
                        260 265 270
Glu Cys Val Pro Thr Glu Glu Ser Asn Ile Thr Met Gln Ile Met Arg
                275 280 285
Ile Lys Pro His Gln Gly Gln His Ile Gly Glu Met Ser Phe Leu Gln
        290 295 300
His Asn Lys Cys Glu Cys Arg Pro Lys Lys Asp Arg Ala Arg Gln Glu
305 310 315 320
Lys Lys Ser Val Arg Gly Lys Gly Lys Gly Gln Lys Arg Lys Arg Lys
                                325 330 335
Lys Ser Arg Tyr Lys Ser Trp Ser Val Pro Cys Gly Pro Cys Ser Glu
                        340 345 350
Arg Arg Lys His Leu Phe Val Gln Asp Pro Gln Thr Cys Lys Cys Ser
                355 360 365
Cys Lys Asn Thr Asp Ser Arg Cys Lys Ala Arg Gln Leu Glu Leu Asn
        370 375 380
Glu Arg Thr Cys Arg Cys Asp Lys Pro Arg Arg
385 390 395
<210> SEQ ID NO 100
<211> LENGTH: 209
<212> TYPE: PRT
<213> ORGANISM: Homo sapiens
<400> SEQUENCE: 100
Met Asn Phe Leu Leu Ser Trp Val His Trp Ser Leu Ala Leu Leu Leu
1 5 10 15
Tyr Leu His His Ala Lys Trp Ser Gln Ala Ala Pro Met Ala Glu Gly
                        20 25 30
Gly Gly Gln Asn His His Glu Val Val Lys Phe Met Asp Val Tyr Gln
                35 40 45
Arg Ser Tyr Cys His Pro Ile Glu Thr Leu Val Asp Ile Phe Gln Glu
        50 55 60
Tyr Pro Asp Glu Ile Glu Tyr Ile Phe Lys Pro Ser Cys Val Pro Leu
65 70 75 80
Met Arg Cys Gly Gly Cys Cys Asn Asp Glu Gly Leu Glu Cys Val Pro
                                85 90 95
Thr Glu Glu Ser Asn Ile Thr Met Gln Ile Met Arg Ile Lys Pro His
                        100 105 110
Gln Gly Gln His Ile Gly Glu Met Ser Phe Leu Gln His Asn Lys Cys
                115 120 125
Glu Cys Arg Pro Lys Lys Asp Arg Ala Arg Gln Glu Lys Lys Ser Val
        130 135 140
Arg Gly Lys Gly Lys Gly Gln Lys Arg Lys Arg Lys Lys Ser Arg Pro
145 150 155 160
Cys Gly Pro Cys Ser Glu Arg Arg Lys His Leu Phe Val Gln Asp Pro
                                165 170 175
Gln Thr Cys Lys Cys Ser Cys Lys Asn Thr Asp Ser Arg Cys Lys Ala
                        180 185 190
Arg Gln Leu Glu Leu Asn Glu Arg Thr Cys Arg Cys Asp Lys Pro Arg
                195 200 205
Arg
<210> SEQ ID NO 101
<211> LENGTH: 389
<212> TYPE: PRT
<213> ORGANISM: Homo sapiens
<400> SEQUENCE: 101
Met Thr Asp Arg Gln Thr Asp Thr Ala Pro Ser Pro Ser Tyr His Leu
1 5 10 15
Leu Pro Gly Arg Arg Arg Thr Val Asp Ala Ala Ala Ser Arg Gly Gln
                        20 25 30
Gly Pro Glu Pro Ala Pro Gly Gly Gly Val Glu Gly Val Gly Ala Arg
                35 40 45
Gly Val Ala Leu Lys Leu Phe Val Gln Leu Leu Gly Cys Ser Arg Phe
        50 55 60
Gly Gly Ala Val Val Arg Ala Gly Glu Ala Glu Pro Ser Gly Ala Ala
65 70 75 80
Arg Ser Ala Ser Ser Gly Arg Glu Glu Pro Gln Pro Glu Glu Gly Glu
                                85 90 95
Glu Glu Glu Glu Lys Glu Glu Glu Arg Gly Pro Gln Trp Arg Leu Gly
                        100 105 110
Ala Arg Lys Pro Gly Ser Trp Thr Gly Glu Ala Ala Val Cys Ala Asp
                115 120 125
Ser Ala Pro Ala Ala Arg Ala Pro Gln Ala Leu Ala Arg Ala Ser Gly
        130 135 140
Arg Gly Gly Arg Val Ala Arg Arg Gly Ala Glu Glu Ser Gly Pro Pro
145 150 155 160
His Ser Pro Ser Arg Arg Gly Ser Ala Ser Arg Ala Gly Pro Gly Arg
                                165 170 175
Ala Ser Glu Thr Met Asn Phe Leu Leu Ser Trp Val His Trp Ser Leu
                        180 185 190
Ala Leu Leu Leu Tyr Leu His His Ala Lys Trp Ser Gln Ala Ala Pro
                195 200 205
Met Ala Glu Gly Gly Gly Gln Asn His His Glu Val Val Lys Phe Met
        210 215 220
Asp Val Tyr Gln Arg Ser Tyr Cys His Pro Ile Glu Thr Leu Val Asp
225 230 235 240
Ile Phe Gln Glu Tyr Pro Asp Glu Ile Glu Tyr Ile Phe Lys Pro Ser
                                245 250 255
Cys Val Pro Leu Met Arg Cys Gly Gly Cys Cys Asn Asp Glu Gly Leu
                        260 265 270
Glu Cys Val Pro Thr Glu Glu Ser Asn Ile Thr Met Gln Ile Met Arg
                275 280 285
Ile Lys Pro His Gln Gly Gln His Ile Gly Glu Met Ser Phe Leu Gln
        290 295 300
His Asn Lys Cys Glu Cys Arg Pro Lys Lys Asp Arg Ala Arg Gln Glu
305 310 315 320
Lys Lys Ser Val Arg Gly Lys Gly Lys Gly Gln Lys Arg Lys Arg Lys
                                325 330 335
Lys Ser Arg Pro Cys Gly Pro Cys Ser Glu Arg Arg Lys His Leu Phe
                        340 345 350
Val Gln Asp Pro Gln Thr Cys Lys Cys Ser Cys Lys Asn Thr Asp Ser
                355 360 365
Arg Cys Lys Ala Arg Gln Leu Glu Leu Asn Glu Arg Thr Cys Arg Cys
        370 375 380
Asp Lys Pro Arg Arg
385
<210> SEQ ID NO 102
<211> LENGTH: 191
<212> TYPE: PRT
<213> ORGANISM: Homo sapiens
<400> SEQUENCE: 102
Met Asn Phe Leu Leu Ser Trp Val His Trp Ser Leu Ala Leu Leu Leu
1 5 10 15
Tyr Leu His His Ala Lys Trp Ser Gln Ala Ala Pro Met Ala Glu Gly
                        20 25 30
Gly Gly Gln Asn His His Glu Val Val Lys Phe Met Asp Val Tyr Gln
                35 40 45
Arg Ser Tyr Cys His Pro Ile Glu Thr Leu Val Asp Ile Phe Gln Glu
        50 55 60
Tyr Pro Asp Glu Ile Glu Tyr Ile Phe Lys Pro Ser Cys Val Pro Leu
65 70 75 80
Met Arg Cys Gly Gly Cys Cys Asn Asp Glu Gly Leu Glu Cys Val Pro
                                85 90 95
Thr Glu Glu Ser Asn Ile Thr Met Gln Ile Met Arg Ile Lys Pro His
                        100 105 110
Gln Gly Gln His Ile Gly Glu Met Ser Phe Leu Gln His Asn Lys Cys
                115 120 125
Glu Cys Arg Pro Lys Lys Asp Arg Ala Arg Gln Glu Asn Pro Cys Gly
        130 135 140
Pro Cys Ser Glu Arg Arg Lys His Leu Phe Val Gln Asp Pro Gln Thr
145 150 155 160
Cys Lys Cys Ser Cys Lys Asn Thr Asp Ser Arg Cys Lys Ala Arg Gln
                                165 170 175
Leu Glu Leu Asn Glu Arg Thr Cys Arg Cys Asp Lys Pro Arg Arg
                        180 185 190
<210> SEQ ID NO 103
<211> LENGTH: 371
<212> TYPE: PRT
<213> ORGANISM: Homo sapiens
<400> SEQUENCE: 103
Met Thr Asp Arg Gln Thr Asp Thr Ala Pro Ser Pro Ser Tyr His Leu
1 5 10 15
Leu Pro Gly Arg Arg Arg Thr Val Asp Ala Ala Ala Ser Arg Gly Gln
                        20 25 30
Gly Pro Glu Pro Ala Pro Gly Gly Gly Val Glu Gly Val Gly Ala Arg
                35 40 45
Gly Val Ala Leu Lys Leu Phe Val Gln Leu Leu Gly Cys Ser Arg Phe
        50 55 60
Gly Gly Ala Val Val Arg Ala Gly Glu Ala Glu Pro Ser Gly Ala Ala
65 70 75 80
Arg Ser Ala Ser Ser Gly Arg Glu Glu Pro Gln Pro Glu Glu Gly Glu
                                85 90 95
Glu Glu Glu Glu Lys Glu Glu Glu Arg Gly Pro Gln Trp Arg Leu Gly
                        100 105 110
Ala Arg Lys Pro Gly Ser Trp Thr Gly Glu Ala Ala Val Cys Ala Asp
                115 120 125
Ser Ala Pro Ala Ala Arg Ala Pro Gln Ala Leu Ala Arg Ala Ser Gly
        130 135 140
Arg Gly Gly Arg Val Ala Arg Arg Gly Ala Glu Glu Ser Gly Pro Pro
145 150 155 160
His Ser Pro Ser Arg Arg Gly Ser Ala Ser Arg Ala Gly Pro Gly Arg
                                165 170 175
Ala Ser Glu Thr Met Asn Phe Leu Leu Ser Trp Val His Trp Ser Leu
                        180 185 190
Ala Leu Leu Leu Tyr Leu His His Ala Lys Trp Ser Gln Ala Ala Pro
                195 200 205
Met Ala Glu Gly Gly Gly Gln Asn His His Glu Val Val Lys Phe Met
        210 215 220
Asp Val Tyr Gln Arg Ser Tyr Cys His Pro Ile Glu Thr Leu Val Asp
225 230 235 240
Ile Phe Gln Glu Tyr Pro Asp Glu Ile Glu Tyr Ile Phe Lys Pro Ser
                                245 250 255
Cys Val Pro Leu Met Arg Cys Gly Gly Cys Cys Asn Asp Glu Gly Leu
                        260 265 270
Glu Cys Val Pro Thr Glu Glu Ser Asn Ile Thr Met Gln Ile Met Arg
                275 280 285
Ile Lys Pro His Gln Gly Gln His Ile Gly Glu Met Ser Phe Leu Gln
        290 295 300
His Asn Lys Cys Glu Cys Arg Pro Lys Lys Asp Arg Ala Arg Gln Glu
305 310 315 320
Asn Pro Cys Gly Pro Cys Ser Glu Arg Arg Lys His Leu Phe Val Gln
                                325 330 335
Asp Pro Gln Thr Cys Lys Cys Ser Cys Lys Asn Thr Asp Ser Arg Cys
                        340 345 350
Lys Ala Arg Gln Leu Glu Leu Asn Glu Arg Thr Cys Arg Cys Asp Lys
                355 360 365
Pro Arg Arg
        370
<210> SEQ ID NO 104
<211> LENGTH: 174
<212> TYPE: PRT
<213> ORGANISM: Homo sapiens
<400> SEQUENCE: 104
Met Asn Phe Leu Leu Ser Trp Val His Trp Ser Leu Ala Leu Leu Leu
1 5 10 15
Tyr Leu His His Ala Lys Trp Ser Gln Ala Ala Pro Met Ala Glu Gly
                        20 25 30
Gly Gly Gln Asn His His Glu Val Val Lys Phe Met Asp Val Tyr Gln
                35 40 45
Arg Ser Tyr Cys His Pro Ile Glu Thr Leu Val Asp Ile Phe Gln Glu
        50 55 60
Tyr Pro Asp Glu Ile Glu Tyr Ile Phe Lys Pro Ser Cys Val Pro Leu
65 70 75 80
Met Arg Cys Gly Gly Cys Cys Asn Asp Glu Gly Leu Glu Cys Val Pro
                                85 90 95
Thr Glu Glu Ser Asn Ile Thr Met Gln Ile Met Arg Ile Lys Pro His
                        100 105 110
Gln Gly Gln His Ile Gly Glu Met Ser Phe Leu Gln His Asn Lys Cys
                115 120 125
Glu Cys Arg Pro Lys Lys Asp Arg Ala Arg Gln Glu Asn Pro Cys Gly
        130 135 140
Pro Cys Ser Glu Arg Arg Lys His Leu Phe Val Gln Asp Pro Gln Thr
145 150 155 160
Cys Lys Cys Ser Cys Lys Asn Thr Asp Ser Arg Cys Lys Met
                                165 170
<210> SEQ ID NO 105
<211> LENGTH: 354
<212> TYPE: PRT
<213> ORGANISM: Homo sapiens
<400> SEQUENCE: 105
Met Thr Asp Arg Gln Thr Asp Thr Ala Pro Ser Pro Ser Tyr His Leu
1 5 10 15
Leu Pro Gly Arg Arg Arg Thr Val Asp Ala Ala Ala Ser Arg Gly Gln
                        20 25 30
Gly Pro Glu Pro Ala Pro Gly Gly Gly Val Glu Gly Val Gly Ala Arg
                35 40 45
Gly Val Ala Leu Lys Leu Phe Val Gln Leu Leu Gly Cys Ser Arg Phe
        50 55 60
Gly Gly Ala Val Val Arg Ala Gly Glu Ala Glu Pro Ser Gly Ala Ala
65 70 75 80
Arg Ser Ala Ser Ser Gly Arg Glu Glu Pro Gln Pro Glu Glu Gly Glu
                                85 90 95
Glu Glu Glu Glu Lys Glu Glu Glu Arg Gly Pro Gln Trp Arg Leu Gly
                        100 105 110
Ala Arg Lys Pro Gly Ser Trp Thr Gly Glu Ala Ala Val Cys Ala Asp
                115 120 125
Ser Ala Pro Ala Ala Arg Ala Pro Gln Ala Leu Ala Arg Ala Ser Gly
        130 135 140
Arg Gly Gly Arg Val Ala Arg Arg Gly Ala Glu Glu Ser Gly Pro Pro
145 150 155 160
His Ser Pro Ser Arg Arg Gly Ser Ala Ser Arg Ala Gly Pro Gly Arg
                                165 170 175
Ala Ser Glu Thr Met Asn Phe Leu Leu Ser Trp Val His Trp Ser Leu
                        180 185 190
Ala Leu Leu Leu Tyr Leu His His Ala Lys Trp Ser Gln Ala Ala Pro
                195 200 205
Met Ala Glu Gly Gly Gly Gln Asn His His Glu Val Val Lys Phe Met
        210 215 220
Asp Val Tyr Gln Arg Ser Tyr Cys His Pro Ile Glu Thr Leu Val Asp
225 230 235 240
Ile Phe Gln Glu Tyr Pro Asp Glu Ile Glu Tyr Ile Phe Lys Pro Ser
                                245 250 255
Cys Val Pro Leu Met Arg Cys Gly Gly Cys Cys Asn Asp Glu Gly Leu
                        260 265 270
Glu Cys Val Pro Thr Glu Glu Ser Asn Ile Thr Met Gln Ile Met Arg
                275 280 285
Ile Lys Pro His Gln Gly Gln His Ile Gly Glu Met Ser Phe Leu Gln
        290 295 300
His Asn Lys Cys Glu Cys Arg Pro Lys Lys Asp Arg Ala Arg Gln Glu
305 310 315 320
Asn Pro Cys Gly Pro Cys Ser Glu Arg Arg Lys His Leu Phe Val Gln
                                325 330 335
Asp Pro Gln Thr Cys Lys Cys Ser Cys Lys Asn Thr Asp Ser Arg Cys
                        340 345 350
Lys Met
<210> SEQ ID NO 106
<211> LENGTH: 147
<212> TYPE: PRT
<213> ORGANISM: Homo sapiens
<400> SEQUENCE: 106
Met Asn Phe Leu Leu Ser Trp Val His Trp Ser Leu Ala Leu Leu Leu
1 5 10 15
Tyr Leu His His Ala Lys Trp Ser Gln Ala Ala Pro Met Ala Glu Gly
                        20 25 30
Gly Gly Gln Asn His His Glu Val Val Lys Phe Met Asp Val Tyr Gln
                35 40 45
Arg Ser Tyr Cys His Pro Ile Glu Thr Leu Val Asp Ile Phe Gln Glu
        50 55 60
Tyr Pro Asp Glu Ile Glu Tyr Ile Phe Lys Pro Ser Cys Val Pro Leu
65 70 75 80
Met Arg Cys Gly Gly Cys Cys Asn Asp Glu Gly Leu Glu Cys Val Pro
                                85 90 95
Thr Glu Glu Ser Asn Ile Thr Met Gln Ile Met Arg Ile Lys Pro His
                        100 105 110
Gln Gly Gln His Ile Gly Glu Met Ser Phe Leu Gln His Asn Lys Cys
                115 120 125
Glu Cys Arg Pro Lys Lys Asp Arg Ala Arg Gln Glu Lys Cys Asp Lys
        130 135 140
Pro Arg Arg
145
<210> SEQ ID NO 107
<211> LENGTH: 327
<212> TYPE: PRT
<213> ORGANISM: Homo sapiens
<400> SEQUENCE: 107
Met Thr Asp Arg Gln Thr Asp Thr Ala Pro Ser Pro Ser Tyr His Leu
1 5 10 15
Leu Pro Gly Arg Arg Arg Thr Val Asp Ala Ala Ala Ser Arg Gly Gln
                        20 25 30
Gly Pro Glu Pro Ala Pro Gly Gly Gly Val Glu Gly Val Gly Ala Arg
                35 40 45
Gly Val Ala Leu Lys Leu Phe Val Gln Leu Leu Gly Cys Ser Arg Phe
        50 55 60
Gly Gly Ala Val Val Arg Ala Gly Glu Ala Glu Pro Ser Gly Ala Ala
65 70 75 80
Arg Ser Ala Ser Ser Gly Arg Glu Glu Pro Gln Pro Glu Glu Gly Glu
                                85 90 95
Glu Glu Glu Glu Lys Glu Glu Glu Arg Gly Pro Gln Trp Arg Leu Gly
                        100 105 110
Ala Arg Lys Pro Gly Ser Trp Thr Gly Glu Ala Ala Val Cys Ala Asp
                115 120 125
Ser Ala Pro Ala Ala Arg Ala Pro Gln Ala Leu Ala Arg Ala Ser Gly
        130 135 140
Arg Gly Gly Arg Val Ala Arg Arg Gly Ala Glu Glu Ser Gly Pro Pro
145 150 155 160
His Ser Pro Ser Arg Arg Gly Ser Ala Ser Arg Ala Gly Pro Gly Arg
                                165 170 175
Ala Ser Glu Thr Met Asn Phe Leu Leu Ser Trp Val His Trp Ser Leu
                        180 185 190
Ala Leu Leu Leu Tyr Leu His His Ala Lys Trp Ser Gln Ala Ala Pro
                195 200 205
Met Ala Glu Gly Gly Gly Gln Asn His His Glu Val Val Lys Phe Met
        210 215 220
Asp Val Tyr Gln Arg Ser Tyr Cys His Pro Ile Glu Thr Leu Val Asp
225 230 235 240
Ile Phe Gln Glu Tyr Pro Asp Glu Ile Glu Tyr Ile Phe Lys Pro Ser
                                245 250 255
Cys Val Pro Leu Met Arg Cys Gly Gly Cys Cys Asn Asp Glu Gly Leu
                        260 265 270
Glu Cys Val Pro Thr Glu Glu Ser Asn Ile Thr Met Gln Ile Met Arg
                275 280 285
Ile Lys Pro His Gln Gly Gln His Ile Gly Glu Met Ser Phe Leu Gln
        290 295 300
His Asn Lys Cys Glu Cys Arg Pro Lys Lys Asp Arg Ala Arg Gln Glu
305 310 315 320
Lys Cys Asp Lys Pro Arg Arg
                                325
<210> SEQ ID NO 108
<211> LENGTH: 191
<212> TYPE: PRT
<213> ORGANISM: Homo sapiens
<400> SEQUENCE: 108
Met Asn Phe Leu Leu Ser Trp Val His Trp Ser Leu Ala Leu Leu Leu
1 5 10 15
Tyr Leu His His Ala Lys Trp Ser Gln Ala Ala Pro Met Ala Glu Gly
                        20 25 30
Gly Gly Gln Asn His His Glu Val Val Lys Phe Met Asp Val Tyr Gln
                35 40 45
Arg Ser Tyr Cys His Pro Ile Glu Thr Leu Val Asp Ile Phe Gln Glu
        50 55 60
Tyr Pro Asp Glu Ile Glu Tyr Ile Phe Lys Pro Ser Cys Val Pro Leu
65 70 75 80
Met Arg Cys Gly Gly Cys Cys Asn Asp Glu Gly Leu Glu Cys Val Pro
                                85 90 95
Thr Glu Glu Ser Asn Ile Thr Met Gln Ile Met Arg Ile Lys Pro His
                        100 105 110
Gln Gly Gln His Ile Gly Glu Met Ser Phe Leu Gln His Asn Lys Cys
                115 120 125
Glu Cys Arg Pro Lys Lys Asp Arg Ala Arg Gln Glu Asn Pro Cys Gly
        130 135 140
Pro Cys Ser Glu Arg Arg Lys His Leu Phe Val Gln Asp Pro Gln Thr
145 150 155 160
Cys Lys Cys Ser Cys Lys Asn Thr Asp Ser Arg Cys Lys Ala Arg Gln
                                165 170 175
Leu Glu Leu Asn Glu Arg Thr Cys Arg Ser Leu Thr Arg Lys Asp
                        180 185 190
<210> SEQ ID NO 109
<211> LENGTH: 371
<212> TYPE: PRT
<213> ORGANISM: Homo sapiens
<400> SEQUENCE: 109
Met Thr Asp Arg Gln Thr Asp Thr Ala Pro Ser Pro Ser Tyr His Leu
1 5 10 15
Leu Pro Gly Arg Arg Arg Thr Val Asp Ala Ala Ala Ser Arg Gly Gln
                        20 25 30
Gly Pro Glu Pro Ala Pro Gly Gly Gly Val Glu Gly Val Gly Ala Arg
                35 40 45
Gly Val Ala Leu Lys Leu Phe Val Gln Leu Leu Gly Cys Ser Arg Phe
        50 55 60
Gly Gly Ala Val Val Arg Ala Gly Glu Ala Glu Pro Ser Gly Ala Ala
65 70 75 80
Arg Ser Ala Ser Ser Gly Arg Glu Glu Pro Gln Pro Glu Glu Gly Glu
                                85 90 95
Glu Glu Glu Glu Lys Glu Glu Glu Arg Gly Pro Gln Trp Arg Leu Gly
                        100 105 110
Ala Arg Lys Pro Gly Ser Trp Thr Gly Glu Ala Ala Val Cys Ala Asp
                115 120 125
Ser Ala Pro Ala Ala Arg Ala Pro Gln Ala Leu Ala Arg Ala Ser Gly
        130 135 140
Arg Gly Gly Arg Val Ala Arg Arg Gly Ala Glu Glu Ser Gly Pro Pro
145 150 155 160
His Ser Pro Ser Arg Arg Gly Ser Ala Ser Arg Ala Gly Pro Gly Arg
                                165 170 175
Ala Ser Glu Thr Met Asn Phe Leu Leu Ser Trp Val His Trp Ser Leu
                        180 185 190
Ala Leu Leu Leu Tyr Leu His His Ala Lys Trp Ser Gln Ala Ala Pro
                195 200 205
Met Ala Glu Gly Gly Gly Gln Asn His His Glu Val Val Lys Phe Met
        210 215 220
Asp Val Tyr Gln Arg Ser Tyr Cys His Pro Ile Glu Thr Leu Val Asp
225 230 235 240
Ile Phe Gln Glu Tyr Pro Asp Glu Ile Glu Tyr Ile Phe Lys Pro Ser
                                245 250 255
Cys Val Pro Leu Met Arg Cys Gly Gly Cys Cys Asn Asp Glu Gly Leu
                        260 265 270
Glu Cys Val Pro Thr Glu Glu Ser Asn Ile Thr Met Gln Ile Met Arg
                275 280 285
Ile Lys Pro His Gln Gly Gln His Ile Gly Glu Met Ser Phe Leu Gln
        290 295 300
His Asn Lys Cys Glu Cys Arg Pro Lys Lys Asp Arg Ala Arg Gln Glu
305 310 315 320
Asn Pro Cys Gly Pro Cys Ser Glu Arg Arg Lys His Leu Phe Val Gln
                                325 330 335
Asp Pro Gln Thr Cys Lys Cys Ser Cys Lys Asn Thr Asp Ser Arg Cys
                        340 345 350
Lys Ala Arg Gln Leu Glu Leu Asn Glu Arg Thr Cys Arg Ser Leu Thr
                355 360 365
Arg Lys Asp
        370
<210> SEQ ID NO 110
<211> LENGTH: 137
<212> TYPE: PRT
<213> ORGANISM: Homo sapiens
<400> SEQUENCE: 110
Met Asn Phe Leu Leu Ser Trp Val His Trp Ser Leu Ala Leu Leu Leu
1 5 10 15
Tyr Leu His His Ala Lys Trp Ser Gln Ala Ala Pro Met Ala Glu Gly
                        20 25 30
Gly Gly Gln Asn His His Glu Val Val Lys Phe Met Asp Val Tyr Gln
                35 40 45
Arg Ser Tyr Cys His Pro Ile Glu Thr Leu Val Asp Ile Phe Gln Glu
        50 55 60
Tyr Pro Asp Glu Ile Glu Tyr Ile Phe Lys Pro Ser Cys Val Pro Leu
65 70 75 80
Met Arg Cys Gly Gly Cys Cys Asn Asp Glu Gly Leu Glu Cys Val Pro
                                85 90 95
Thr Glu Glu Ser Asn Ile Thr Met Gln Ile Met Arg Ile Lys Pro His
                        100 105 110
Gln Gly Gln His Ile Gly Glu Met Ser Phe Leu Gln His Asn Lys Cys
                115 120 125
Glu Cys Arg Cys Asp Lys Pro Arg Arg
        130 135
<210> SEQ ID NO 111
<211> LENGTH: 317
<212> TYPE: PRT
<213> ORGANISM: Homo sapiens
<400> SEQUENCE: 111
Met Thr Asp Arg Gln Thr Asp Thr Ala Pro Ser Pro Ser Tyr His Leu
1 5 10 15
Leu Pro Gly Arg Arg Arg Thr Val Asp Ala Ala Ala Ser Arg Gly Gln
                        20 25 30
Gly Pro Glu Pro Ala Pro Gly Gly Gly Val Glu Gly Val Gly Ala Arg
                35 40 45
Gly Val Ala Leu Lys Leu Phe Val Gln Leu Leu Gly Cys Ser Arg Phe
        50 55 60
Gly Gly Ala Val Val Arg Ala Gly Glu Ala Glu Pro Ser Gly Ala Ala
65 70 75 80
Arg Ser Ala Ser Ser Gly Arg Glu Glu Pro Gln Pro Glu Glu Gly Glu
                                85 90 95
Glu Glu Glu Glu Lys Glu Glu Glu Arg Gly Pro Gln Trp Arg Leu Gly
                        100 105 110
Ala Arg Lys Pro Gly Ser Trp Thr Gly Glu Ala Ala Val Cys Ala Asp
                115 120 125
Ser Ala Pro Ala Ala Arg Ala Pro Gln Ala Leu Ala Arg Ala Ser Gly
        130 135 140
Arg Gly Gly Arg Val Ala Arg Arg Gly Ala Glu Glu Ser Gly Pro Pro
145 150 155 160
His Ser Pro Ser Arg Arg Gly Ser Ala Ser Arg Ala Gly Pro Gly Arg
                                165 170 175
Ala Ser Glu Thr Met Asn Phe Leu Leu Ser Trp Val His Trp Ser Leu
                        180 185 190
Ala Leu Leu Leu Tyr Leu His His Ala Lys Trp Ser Gln Ala Ala Pro
                195 200 205
Met Ala Glu Gly Gly Gly Gln Asn His His Glu Val Val Lys Phe Met
        210 215 220
Asp Val Tyr Gln Arg Ser Tyr Cys His Pro Ile Glu Thr Leu Val Asp
225 230 235 240
Ile Phe Gln Glu Tyr Pro Asp Glu Ile Glu Tyr Ile Phe Lys Pro Ser
                                245 250 255
Cys Val Pro Leu Met Arg Cys Gly Gly Cys Cys Asn Asp Glu Gly Leu
                        260 265 270
Glu Cys Val Pro Thr Glu Glu Ser Asn Ile Thr Met Gln Ile Met Arg
                275 280 285
Ile Lys Pro His Gln Gly Gln His Ile Gly Glu Met Ser Phe Leu Gln
        290 295 300
His Asn Lys Cys Glu Cys Arg Cys Asp Lys Pro Arg Arg
305 310 315
<210> SEQ ID NO 112
<211> LENGTH: 351
<212> TYPE: PRT
<213> ORGANISM: Homo sapiens
<400> SEQUENCE: 112
Met Thr Asp Arg Gln Thr Asp Thr Ala Pro Ser Pro Ser Tyr His Leu
1 5 10 15
Leu Pro Gly Arg Arg Arg Thr Val Asp Ala Ala Ala Ser Arg Gly Gln
                        20 25 30
Gly Pro Glu Pro Ala Pro Gly Gly Gly Val Glu Gly Val Gly Ala Arg
                35 40 45
Gly Val Ala Leu Lys Leu Phe Val Gln Leu Leu Gly Cys Ser Arg Phe
        50 55 60
Gly Gly Ala Val Val Arg Ala Gly Glu Ala Glu Pro Ser Gly Ala Ala
65 70 75 80
Arg Ser Ala Ser Ser Gly Arg Glu Glu Pro Gln Pro Glu Glu Gly Glu
                                85 90 95
Glu Glu Glu Glu Lys Glu Glu Glu Arg Gly Pro Gln Trp Arg Leu Gly
                        100 105 110
Ala Arg Lys Pro Gly Ser Trp Thr Gly Glu Ala Ala Val Cys Ala Asp
                115 120 125
Ser Ala Pro Ala Ala Arg Ala Pro Gln Ala Leu Ala Arg Ala Ser Gly
        130 135 140
Arg Gly Gly Arg Val Ala Arg Arg Gly Ala Glu Glu Ser Gly Pro Pro
145 150 155 160
His Ser Pro Ser Arg Arg Gly Ser Ala Ser Arg Ala Gly Pro Gly Arg
                                165 170 175
Ala Ser Glu Thr Met Asn Phe Leu Leu Ser Trp Val His Trp Ser Leu
                        180 185 190
Ala Leu Leu Leu Tyr Leu His His Ala Lys Trp Ser Gln Ala Ala Pro
                195 200 205
Met Ala Glu Gly Gly Gly Gln Asn His His Glu Val Val Lys Phe Met
        210 215 220
Asp Val Tyr Gln Arg Ser Tyr Cys His Pro Ile Glu Thr Leu Val Asp
225 230 235 240
Ile Phe Gln Glu Tyr Pro Asp Glu Ile Glu Tyr Ile Phe Lys Pro Ser
                                245 250 255
Cys Val Pro Leu Met Arg Cys Gly Gly Cys Cys Asn Asp Glu Gly Leu
                        260 265 270
Glu Cys Val Pro Thr Glu Glu Ser Asn Ile Thr Met Gln Ile Met Arg
                275 280 285
Ile Lys Pro His Gln Gly Gln His Ile Gly Glu Met Ser Phe Leu Gln
        290 295 300
His Asn Lys Cys Glu Cys Arg Pro Lys Lys Asp Arg Ala Arg Gln Glu
305 310 315 320
Lys Lys Ser Val Arg Gly Lys Gly Lys Gly Gln Lys Arg Lys Arg Lys
                                325 330 335
Lys Ser Arg Tyr Lys Ser Trp Ser Val Cys Asp Lys Pro Arg Arg
                        340 345 350
<210> SEQ ID NO 113
<211> LENGTH: 351
<212> TYPE: PRT
<213> ORGANISM: Homo sapiens
<400> SEQUENCE: 113
Met Thr Asp Arg Gln Thr Asp Thr Ala Pro Ser Pro Ser Tyr His Leu
1 5 10 15
Leu Pro Gly Arg Arg Arg Thr Val Asp Ala Ala Ala Ser Arg Gly Gln
                        20 25 30
Gly Pro Glu Pro Ala Pro Gly Gly Gly Val Glu Gly Val Gly Ala Arg
                35 40 45
Gly Val Ala Leu Lys Leu Phe Val Gln Leu Leu Gly Cys Ser Arg Phe
        50 55 60
Gly Gly Ala Val Val Arg Ala Gly Glu Ala Glu Pro Ser Gly Ala Ala
65 70 75 80
Arg Ser Ala Ser Ser Gly Arg Glu Glu Pro Gln Pro Glu Glu Gly Glu
                                85 90 95
Glu Glu Glu Glu Lys Glu Glu Glu Arg Gly Pro Gln Trp Arg Leu Gly
                        100 105 110
Ala Arg Lys Pro Gly Ser Trp Thr Gly Glu Ala Ala Val Cys Ala Asp
                115 120 125
Ser Ala Pro Ala Ala Arg Ala Pro Gln Ala Leu Ala Arg Ala Ser Gly
        130 135 140
Arg Gly Gly Arg Val Ala Arg Arg Gly Ala Glu Glu Ser Gly Pro Pro
145 150 155 160
His Ser Pro Ser Arg Arg Gly Ser Ala Ser Arg Ala Gly Pro Gly Arg
                                165 170 175
Ala Ser Glu Thr Met Asn Phe Leu Leu Ser Trp Val His Trp Ser Leu
                        180 185 190
Ala Leu Leu Leu Tyr Leu His His Ala Lys Trp Ser Gln Ala Ala Pro
                195 200 205
Met Ala Glu Gly Gly Gly Gln Asn His His Glu Val Val Lys Phe Met
        210 215 220
Asp Val Tyr Gln Arg Ser Tyr Cys His Pro Ile Glu Thr Leu Val Asp
225 230 235 240
Ile Phe Gln Glu Tyr Pro Asp Glu Ile Glu Tyr Ile Phe Lys Pro Ser
                                245 250 255
Cys Val Pro Leu Met Arg Cys Gly Gly Cys Cys Asn Asp Glu Gly Leu
                        260 265 270
Glu Cys Val Pro Thr Glu Glu Ser Asn Ile Thr Met Gln Ile Met Arg
                275 280 285
Ile Lys Pro His Gln Gly Gln His Ile Gly Glu Met Ser Phe Leu Gln
        290 295 300
His Asn Lys Cys Glu Cys Arg Pro Lys Lys Asp Arg Ala Arg Gln Glu
305 310 315 320
Lys Lys Ser Val Arg Gly Lys Gly Lys Gly Gln Lys Arg Lys Arg Lys
                                325 330 335
Lys Ser Arg Tyr Lys Ser Trp Ser Val Cys Asp Lys Pro Arg Arg
                        340 345 350
<210> SEQ ID NO 114
<211> LENGTH: 171
<212> TYPE: PRT
<213> ORGANISM: Homo sapiens
<400> SEQUENCE: 114
Met Asn Phe Leu Leu Ser Trp Val His Trp Ser Leu Ala Leu Leu Leu
1 5 10 15
Tyr Leu His His Ala Lys Trp Ser Gln Ala Ala Pro Met Ala Glu Gly
                        20 25 30
Gly Gly Gln Asn His His Glu Val Val Lys Phe Met Asp Val Tyr Gln
                35 40 45
Arg Ser Tyr Cys His Pro Ile Glu Thr Leu Val Asp Ile Phe Gln Glu
        50 55 60
Tyr Pro Asp Glu Ile Glu Tyr Ile Phe Lys Pro Ser Cys Val Pro Leu
65 70 75 80
Met Arg Cys Gly Gly Cys Cys Asn Asp Glu Gly Leu Glu Cys Val Pro
                                85 90 95
Thr Glu Glu Ser Asn Ile Thr Met Gln Ile Met Arg Ile Lys Pro His
                        100 105 110
Gln Gly Gln His Ile Gly Glu Met Ser Phe Leu Gln His Asn Lys Cys
                115 120 125
Glu Cys Arg Pro Lys Lys Asp Arg Ala Arg Gln Glu Lys Lys Ser Val
        130 135 140
Arg Gly Lys Gly Lys Gly Gln Lys Arg Lys Arg Lys Lys Ser Arg Tyr
145 150 155 160
Lys Ser Trp Ser Val Cys Asp Lys Pro Arg Arg
                                165 170
<210> SEQ ID NO 115
<211> LENGTH: 188
<212> TYPE: PRT
<213> ORGANISM: Homo sapiens
<400> SEQUENCE: 115
Met Ser Pro Leu Leu Arg Arg Leu Leu Leu Ala Ala Leu Leu Gln Leu
1 5 10 15
Ala Pro Ala Gln Ala Pro Val Ser Gln Pro Asp Ala Pro Gly His Gln
                        20 25 30
Arg Lys Val Val Ser Trp Ile Asp Val Tyr Thr Arg Ala Thr Cys Gln
                35 40 45
Pro Arg Glu Val Val Val Pro Leu Thr Val Glu Leu Met Gly Thr Val
        50 55 60
Ala Lys Gln Leu Val Pro Ser Cys Val Thr Val Gln Arg Cys Gly Gly
65 70 75 80
Cys Cys Pro Asp Asp Gly Leu Glu Cys Val Pro Thr Gly Gln His Gln
                                85 90 95
Val Arg Met Gln Ile Leu Met Ile Arg Tyr Pro Ser Ser Gln Leu Gly
                        100 105 110
Glu Met Ser Leu Glu Glu His Ser Gln Cys Glu Cys Arg Pro Lys Lys
                115 120 125
Lys Asp Ser Ala Val Lys Pro Asp Ser Pro Arg Pro Leu Cys Pro Arg
        130 135 140
Cys Thr Gln His His Gln Arg Pro Asp Pro Arg Thr Cys Arg Cys Arg
145 150 155 160
Cys Arg Arg Arg Ser Phe Leu Arg Cys Gln Gly Arg Gly Leu Glu Leu
                                165 170 175
Asn Pro Asp Thr Cys Arg Cys Arg Lys Leu Arg Arg
                        180 185
<210> SEQ ID NO 116
<211> LENGTH: 419
<212> TYPE: PRT
<213> ORGANISM: Homo sapiens
<400> SEQUENCE: 116
Met His Leu Leu Gly Phe Phe Ser Val Ala Cys Ser Leu Leu Ala Ala
1 5 10 15
Ala Leu Leu Pro Gly Pro Arg Glu Ala Pro Ala Ala Ala Ala Ala Phe
                        20 25 30
Glu Ser Gly Leu Asp Leu Ser Asp Ala Glu Pro Asp Ala Gly Glu Ala
                35 40 45
Thr Ala Tyr Ala Ser Lys Asp Leu Glu Glu Gln Leu Arg Ser Val Ser
        50 55 60
Ser Val Asp Glu Leu Met Thr Val Leu Tyr Pro Glu Tyr Trp Lys Met
65 70 75 80
Tyr Lys Cys Gln Leu Arg Lys Gly Gly Trp Gln His Asn Arg Glu Gln
                                85 90 95
Ala Asn Leu Asn Ser Arg Thr Glu Glu Thr Ile Lys Phe Ala Ala Ala
                        100 105 110
His Tyr Asn Thr Glu Ile Leu Lys Ser Ile Asp Asn Glu Trp Arg Lys
                115 120 125
Thr Gln Cys Met Pro Arg Glu Val Cys Ile Asp Val Gly Lys Glu Phe
        130 135 140
Gly Val Ala Thr Asn Thr Phe Phe Lys Pro Pro Cys Val Ser Val Tyr
145 150 155 160
Arg Cys Gly Gly Cys Cys Asn Ser Glu Gly Leu Gln Cys Met Asn Thr
                                165 170 175
Ser Thr Ser Tyr Leu Ser Lys Thr Leu Phe Glu Ile Thr Val Pro Leu
                        180 185 190
Ser Gln Gly Pro Lys Pro Val Thr Ile Ser Phe Ala Asn His Thr Ser
                195 200 205
Cys Arg Cys Met Ser Lys Leu Asp Val Tyr Arg Gln Val His Ser Ile
        210 215 220
Ile Arg Arg Ser Leu Pro Ala Thr Leu Pro Gln Cys Gln Ala Ala Asn
225 230 235 240
Lys Thr Cys Pro Thr Asn Tyr Met Trp Asn Asn His Ile Cys Arg Cys
                                245 250 255
Leu Ala Gln Glu Asp Phe Met Phe Ser Ser Asp Ala Gly Asp Asp Ser
                        260 265 270
Thr Asp Gly Phe His Asp Ile Cys Gly Pro Asn Lys Glu Leu Asp Glu
                275 280 285
Glu Thr Cys Gln Cys Val Cys Arg Ala Gly Leu Arg Pro Ala Ser Cys
        290 295 300
Gly Pro His Lys Glu Leu Asp Arg Asn Ser Cys Gln Cys Val Cys Lys
305 310 315 320
Asn Lys Leu Phe Pro Ser Gln Cys Gly Ala Asn Arg Glu Phe Asp Glu
                                325 330 335
Asn Thr Cys Gln Cys Val Cys Lys Arg Thr Cys Pro Arg Asn Gln Pro
                        340 345 350
Leu Asn Pro Gly Lys Cys Ala Cys Glu Cys Thr Glu Ser Pro Gln Lys
                355 360 365
Cys Leu Leu Lys Gly Lys Lys Phe His His Gln Thr Cys Ser Cys Tyr
        370 375 380
Arg Arg Pro Cys Thr Asn Arg Gln Lys Ala Cys Glu Pro Gly Phe Ser
385 390 395 400
Tyr Ser Glu Glu Val Cys Arg Cys Val Pro Ser Tyr Trp Lys Arg Pro
                                405 410 415
Gln Met Ser
<210> SEQ ID NO 117
<211> LENGTH: 207
<212> TYPE: PRT
<213> ORGANISM: Homo sapiens
<400> SEQUENCE: 117
Met Ser Pro Leu Leu Arg Arg Leu Leu Leu Ala Ala Leu Leu Gln Leu
1 5 10 15
Ala Pro Ala Gln Ala Pro Val Ser Gln Pro Asp Ala Pro Gly His Gln
                        20 25 30
Arg Lys Val Val Ser Trp Ile Asp Val Tyr Thr Arg Ala Thr Cys Gln
                35 40 45
Pro Arg Glu Val Val Val Pro Leu Thr Val Glu Leu Met Gly Thr Val
        50 55 60
Ala Lys Gln Leu Val Pro Ser Cys Val Thr Val Gln Arg Cys Gly Gly
65 70 75 80
Cys Cys Pro Asp Asp Gly Leu Glu Cys Val Pro Thr Gly Gln His Gln
                                85 90 95
Val Arg Met Gln Ile Leu Met Ile Arg Tyr Pro Ser Ser Gln Leu Gly
                        100 105 110
Glu Met Ser Leu Glu Glu His Ser Gln Cys Glu Cys Arg Pro Lys Lys
                115 120 125
Lys Asp Ser Ala Val Lys Pro Asp Arg Ala Ala Thr Pro His His Arg
        130 135 140
Pro Gln Pro Arg Ser Val Pro Gly Trp Asp Ser Ala Pro Gly Ala Pro
145 150 155 160
Ser Pro Ala Asp Ile Thr His Pro Thr Pro Ala Pro Gly Pro Ser Ala
                                165 170 175
His Ala Ala Pro Ser Thr Thr Ser Ala Leu Thr Pro Gly Pro Ala Ala
                        180 185 190
Ala Ala Ala Asp Ala Ala Ala Ser Ser Val Ala Lys Gly Gly Ala
                195 200 205
<210> SEQ ID NO 118
<211> LENGTH: 194
<212> TYPE: PRT
<213> ORGANISM: Homo sapiens
<400> SEQUENCE: 118
Met His Lys Trp Ile Leu Thr Trp Ile Leu Pro Thr Leu Leu Tyr Arg
1 5 10 15
Ser Cys Phe His Ile Ile Cys Leu Val Gly Thr Ile Ser Leu Ala Cys
                        20 25 30
Asn Asp Met Thr Pro Glu Gln Met Ala Thr Asn Val Asn Cys Ser Ser
                35 40 45
Pro Glu Arg His Thr Arg Ser Tyr Asp Tyr Met Glu Gly Gly Asp Ile
        50 55 60
Arg Val Arg Arg Leu Phe Cys Arg Thr Gln Trp Tyr Leu Arg Ile Asp
65 70 75 80
Lys Arg Gly Lys Val Lys Gly Thr Gln Glu Met Lys Asn Asn Tyr Asn
                                85 90 95
Ile Met Glu Ile Arg Thr Val Ala Val Gly Ile Val Ala Ile Lys Gly
                        100 105 110
Val Glu Ser Glu Phe Tyr Leu Ala Met Asn Lys Glu Gly Lys Leu Tyr
                115 120 125
Ala Lys Lys Glu Cys Asn Glu Asp Cys Asn Phe Lys Glu Leu Ile Leu
        130 135 140
Glu Asn His Tyr Asn Thr Tyr Ala Ser Ala Lys Trp Thr His Asn Gly
145 150 155 160
Gly Glu Met Phe Val Ala Leu Asn Gln Lys Gly Ile Pro Val Arg Gly
                                165 170 175
Lys Lys Thr Lys Lys Glu Gln Lys Thr Ala His Phe Leu Pro Met Ala
                        180 185 190
Ile Thr
<210> SEQ ID NO 119
<211> LENGTH: 160
<212> TYPE: PRT
<213> ORGANISM: Homo sapiens
<400> SEQUENCE: 119
Met Val Pro Ser Ala Gly Gln Leu Ala Leu Phe Ala Leu Gly Ile Val
1 5 10 15
Leu Ala Ala Cys Gln Ala Leu Glu Asn Ser Thr Ser Pro Leu Ser Ala
                        20 25 30
Asp Pro Pro Val Ala Ala Ala Val Val Ser His Phe Asn Asp Cys Pro
                35 40 45
Asp Ser His Thr Gln Phe Cys Phe His Gly Thr Cys Arg Phe Leu Val
        50 55 60
Gln Glu Asp Lys Pro Ala Cys Val Cys His Ser Gly Tyr Val Gly Ala
65 70 75 80
Arg Cys Glu His Ala Asp Leu Leu Ala Val Val Ala Ala Ser Gln Lys
                                85 90 95
Lys Gln Ala Ile Thr Ala Leu Val Val Val Ser Ile Val Ala Leu Ala
                        100 105 110
Val Leu Ile Ile Thr Cys Val Leu Ile His Cys Cys Gln Val Arg Lys
                115 120 125
His Cys Glu Trp Cys Arg Ala Leu Ile Cys Arg His Glu Lys Pro Ser
        130 135 140
Ala Leu Leu Lys Gly Arg Thr Ala Cys Cys His Ser Glu Thr Val Val
145 150 155 160
<210> SEQ ID NO 120
<211> LENGTH: 159
<212> TYPE: PRT
<213> ORGANISM: Homo sapiens
<400> SEQUENCE: 120
Met Val Pro Ser Ala Gly Gln Leu Ala Leu Phe Ala Leu Gly Ile Val
1 5 10 15
Leu Ala Ala Cys Gln Ala Leu Glu Asn Ser Thr Ser Pro Leu Ser Asp
                        20 25 30
Pro Pro Val Ala Ala Ala Val Val Ser His Phe Asn Asp Cys Pro Asp
                35 40 45
Ser His Thr Gln Phe Cys Phe His Gly Thr Cys Arg Phe Leu Val Gln
        50 55 60
Glu Asp Lys Pro Ala Cys Val Cys His Ser Gly Tyr Val Gly Ala Arg
65 70 75 80
Cys Glu His Ala Asp Leu Leu Ala Val Val Ala Ala Ser Gln Lys Lys
                                85 90 95
Gln Ala Ile Thr Ala Leu Val Val Val Ser Ile Val Ala Leu Ala Val
                        100 105 110
Leu Ile Ile Thr Cys Val Leu Ile His Cys Cys Gln Val Arg Lys His
                115 120 125
Cys Glu Trp Cys Arg Ala Leu Ile Cys Arg His Glu Lys Pro Ser Ala
        130 135 140
Leu Leu Lys Gly Arg Thr Ala Cys Cys His Ser Glu Thr Val Val
145 150 155
<210> SEQ ID NO 121
<211> LENGTH: 390
<212> TYPE: PRT
<213> ORGANISM: Homo sapiens
<400> SEQUENCE: 121
Met Pro Pro Ser Gly Leu Arg Leu Leu Pro Leu Leu Leu Pro Leu Leu
1 5 10 15
Trp Leu Leu Val Leu Thr Pro Gly Arg Pro Ala Ala Gly Leu Ser Thr
                        20 25 30
Cys Lys Thr Ile Asp Met Glu Leu Val Lys Arg Lys Arg Ile Glu Ala
                35 40 45
Ile Arg Gly Gln Ile Leu Ser Lys Leu Arg Leu Ala Ser Pro Pro Ser
        50 55 60
Gln Gly Glu Val Pro Pro Gly Pro Leu Pro Glu Ala Val Leu Ala Leu
65 70 75 80
Tyr Asn Ser Thr Arg Asp Arg Val Ala Gly Glu Ser Ala Glu Pro Glu
                                85 90 95
Pro Glu Pro Glu Ala Asp Tyr Tyr Ala Lys Glu Val Thr Arg Val Leu
                        100 105 110
Met Val Glu Thr His Asn Glu Ile Tyr Asp Lys Phe Lys Gln Ser Thr
                115 120 125
His Ser Ile Tyr Met Phe Phe Asn Thr Ser Glu Leu Arg Glu Ala Val
        130 135 140
Pro Glu Pro Val Leu Leu Ser Arg Ala Glu Leu Arg Leu Leu Arg Leu
145 150 155 160
Lys Leu Lys Val Glu Gln His Val Glu Leu Tyr Gln Lys Tyr Ser Asn
                                165 170 175
Asn Ser Trp Arg Tyr Leu Ser Asn Arg Leu Leu Ala Pro Ser Asp Ser
                        180 185 190
Pro Glu Trp Leu Ser Phe Asp Val Thr Gly Val Val Arg Gln Trp Leu
                195 200 205
Ser Arg Gly Gly Glu Ile Glu Gly Phe Arg Leu Ser Ala His Cys Ser
        210 215 220
Cys Asp Ser Arg Asp Asn Thr Leu Gln Val Asp Ile Asn Gly Phe Thr
225 230 235 240
Thr Gly Arg Arg Gly Asp Leu Ala Thr Ile His Gly Met Asn Arg Pro
                                245 250 255
Phe Leu Leu Leu Met Ala Thr Pro Leu Glu Arg Ala Gln His Leu Gln
                        260 265 270
Ser Ser Arg His Arg Arg Ala Leu Asp Thr Asn Tyr Cys Phe Ser Ser
                275 280 285
Thr Glu Lys Asn Cys Cys Val Arg Gln Leu Tyr Ile Asp Phe Arg Lys
        290 295 300
Asp Leu Gly Trp Lys Trp Ile His Glu Pro Lys Gly Tyr His Ala Asn
305 310 315 320
Phe Cys Leu Gly Pro Cys Pro Tyr Ile Trp Ser Leu Asp Thr Gln Tyr
                                325 330 335
Ser Lys Val Leu Ala Leu Tyr Asn Gln His Asn Pro Gly Ala Ser Ala
                        340 345 350
Ala Pro Cys Cys Val Pro Gln Ala Leu Glu Pro Leu Pro Ile Val Tyr
                355 360 365
Tyr Val Gly Arg Lys Pro Lys Val Glu Gln Leu Ser Asn Met Ile Val
        370 375 380
Arg Ser Cys Lys Cys Ser
385 390
<210> SEQ ID NO 122
<211> LENGTH: 442
<212> TYPE: PRT
<213> ORGANISM: Homo sapiens
<400> SEQUENCE: 122
Met His Tyr Cys Val Leu Ser Ala Phe Leu Ile Leu His Leu Val Thr
1 5 10 15
Val Ala Leu Ser Leu Ser Thr Cys Ser Thr Leu Asp Met Asp Gln Phe
                        20 25 30
Met Arg Lys Arg Ile Glu Ala Ile Arg Gly Gln Ile Leu Ser Lys Leu
                35 40 45
Lys Leu Thr Ser Pro Pro Glu Asp Tyr Pro Glu Pro Glu Glu Val Pro
        50 55 60
Pro Glu Val Ile Ser Ile Tyr Asn Ser Thr Arg Asp Leu Leu Gln Glu
65 70 75 80
Lys Ala Ser Arg Arg Ala Ala Ala Cys Glu Arg Glu Arg Ser Asp Glu
                                85 90 95
Glu Tyr Tyr Ala Lys Glu Val Tyr Lys Ile Asp Met Pro Pro Phe Phe
                        100 105 110
Pro Ser Glu Thr Val Cys Pro Val Val Thr Thr Pro Ser Gly Ser Val
                115 120 125
Gly Ser Leu Cys Ser Arg Gln Ser Gln Val Leu Cys Gly Tyr Leu Asp
        130 135 140
Ala Ile Pro Pro Thr Phe Tyr Arg Pro Tyr Phe Arg Ile Val Arg Phe
145 150 155 160
Asp Val Ser Ala Met Glu Lys Asn Ala Ser Asn Leu Val Lys Ala Glu
                                165 170 175
Phe Arg Val Phe Arg Leu Gln Asn Pro Lys Ala Arg Val Pro Glu Gln
                        180 185 190
Arg Ile Glu Leu Tyr Gln Ile Leu Lys Ser Lys Asp Leu Thr Ser Pro
                195 200 205
Thr Gln Arg Tyr Ile Asp Ser Lys Val Val Lys Thr Arg Ala Glu Gly
        210 215 220
Glu Trp Leu Ser Phe Asp Val Thr Asp Ala Val His Glu Trp Leu His
225 230 235 240
His Lys Asp Arg Asn Leu Gly Phe Lys Ile Ser Leu His Cys Pro Cys
                                245 250 255
Cys Thr Phe Val Pro Ser Asn Asn Tyr Ile Ile Pro Asn Lys Ser Glu
                        260 265 270
Glu Leu Glu Ala Arg Phe Ala Gly Ile Asp Gly Thr Ser Thr Tyr Thr
                275 280 285
Ser Gly Asp Gln Lys Thr Ile Lys Ser Thr Arg Lys Lys Asn Ser Gly
        290 295 300
Lys Thr Pro His Leu Leu Leu Met Leu Leu Pro Ser Tyr Arg Leu Glu
305 310 315 320
Ser Gln Gln Thr Asn Arg Arg Lys Lys Arg Ala Leu Asp Ala Ala Tyr
                                325 330 335
Cys Phe Arg Asn Val Gln Asp Asn Cys Cys Leu Arg Pro Leu Tyr Ile
                        340 345 350
Asp Phe Lys Arg Asp Leu Gly Trp Lys Trp Ile His Glu Pro Lys Gly
                355 360 365
Tyr Asn Ala Asn Phe Cys Ala Gly Ala Cys Pro Tyr Leu Trp Ser Ser
        370 375 380
Asp Thr Gln His Ser Arg Val Leu Ser Leu Tyr Asn Thr Ile Asn Pro
385 390 395 400
Glu Ala Ser Ala Ser Pro Cys Cys Val Ser Gln Asp Leu Glu Pro Leu
                                405 410 415
Thr Ile Leu Tyr Tyr Ile Gly Lys Thr Pro Lys Ile Glu Gln Leu Ser
                        420 425 430
Asn Met Ile Val Lys Ser Cys Lys Cys Ser
                435 440
<210> SEQ ID NO 123
<211> LENGTH: 414
<212> TYPE: PRT
<213> ORGANISM: Homo sapiens
<400> SEQUENCE: 123
Met His Tyr Cys Val Leu Ser Ala Phe Leu Ile Leu His Leu Val Thr
1 5 10 15
Val Ala Leu Ser Leu Ser Thr Cys Ser Thr Leu Asp Met Asp Gln Phe
                        20 25 30
Met Arg Lys Arg Ile Glu Ala Ile Arg Gly Gln Ile Leu Ser Lys Leu
                35 40 45
Lys Leu Thr Ser Pro Pro Glu Asp Tyr Pro Glu Pro Glu Glu Val Pro
        50 55 60
Pro Glu Val Ile Ser Ile Tyr Asn Ser Thr Arg Asp Leu Leu Gln Glu
65 70 75 80
Lys Ala Ser Arg Arg Ala Ala Ala Cys Glu Arg Glu Arg Ser Asp Glu
                                85 90 95
Glu Tyr Tyr Ala Lys Glu Val Tyr Lys Ile Asp Met Pro Pro Phe Phe
                        100 105 110
Pro Ser Glu Asn Ala Ile Pro Pro Thr Phe Tyr Arg Pro Tyr Phe Arg
                115 120 125
Ile Val Arg Phe Asp Val Ser Ala Met Glu Lys Asn Ala Ser Asn Leu
        130 135 140
Val Lys Ala Glu Phe Arg Val Phe Arg Leu Gln Asn Pro Lys Ala Arg
145 150 155 160
Val Pro Glu Gln Arg Ile Glu Leu Tyr Gln Ile Leu Lys Ser Lys Asp
                                165 170 175
Leu Thr Ser Pro Thr Gln Arg Tyr Ile Asp Ser Lys Val Val Lys Thr
                        180 185 190
Arg Ala Glu Gly Glu Trp Leu Ser Phe Asp Val Thr Asp Ala Val His
                195 200 205
Glu Trp Leu His His Lys Asp Arg Asn Leu Gly Phe Lys Ile Ser Leu
        210 215 220
His Cys Pro Cys Cys Thr Phe Val Pro Ser Asn Asn Tyr Ile Ile Pro
225 230 235 240
Asn Lys Ser Glu Glu Leu Glu Ala Arg Phe Ala Gly Ile Asp Gly Thr
                                245 250 255
Ser Thr Tyr Thr Ser Gly Asp Gln Lys Thr Ile Lys Ser Thr Arg Lys
                        260 265 270
Lys Asn Ser Gly Lys Thr Pro His Leu Leu Leu Met Leu Leu Pro Ser
                275 280 285
Tyr Arg Leu Glu Ser Gln Gln Thr Asn Arg Arg Lys Lys Arg Ala Leu
        290 295 300
Asp Ala Ala Tyr Cys Phe Arg Asn Val Gln Asp Asn Cys Cys Leu Arg
305 310 315 320
Pro Leu Tyr Ile Asp Phe Lys Arg Asp Leu Gly Trp Lys Trp Ile His
                                325 330 335
Glu Pro Lys Gly Tyr Asn Ala Asn Phe Cys Ala Gly Ala Cys Pro Tyr
                        340 345 350
Leu Trp Ser Ser Asp Thr Gln His Ser Arg Val Leu Ser Leu Tyr Asn
                355 360 365
Thr Ile Asn Pro Glu Ala Ser Ala Ser Pro Cys Cys Val Ser Gln Asp
        370 375 380
Leu Glu Pro Leu Thr Ile Leu Tyr Tyr Ile Gly Lys Thr Pro Lys Ile
385 390 395 400
Glu Gln Leu Ser Asn Met Ile Val Lys Ser Cys Lys Cys Ser
                                405 410
<210> SEQ ID NO 124
<211> LENGTH: 412
<212> TYPE: PRT
<213> ORGANISM: Homo sapiens
<400> SEQUENCE: 124
Met Lys Met His Leu Gln Arg Ala Leu Val Val Leu Ala Leu Leu Asn
1 5 10 15
Phe Ala Thr Val Ser Leu Ser Leu Ser Thr Cys Thr Thr Leu Asp Phe
                        20 25 30
Gly His Ile Lys Lys Lys Arg Val Glu Ala Ile Arg Gly Gln Ile Leu
                35 40 45
Ser Lys Leu Arg Leu Thr Ser Pro Pro Glu Pro Thr Val Met Thr His
        50 55 60
Val Pro Tyr Gln Val Leu Ala Leu Tyr Asn Ser Thr Arg Glu Leu Leu
65 70 75 80
Glu Glu Met His Gly Glu Arg Glu Glu Gly Cys Thr Gln Glu Asn Thr
                                85 90 95
Glu Ser Glu Tyr Tyr Ala Lys Glu Ile His Lys Phe Asp Met Ile Gln
                        100 105 110
Gly Leu Ala Glu His Asn Glu Leu Ala Val Cys Pro Lys Gly Ile Thr
                115 120 125
Ser Lys Val Phe Arg Phe Asn Val Ser Ser Val Glu Lys Asn Arg Thr
        130 135 140
Asn Leu Phe Arg Ala Glu Phe Arg Val Leu Arg Val Pro Asn Pro Ser
145 150 155 160
Ser Lys Arg Asn Glu Gln Arg Ile Glu Leu Phe Gln Ile Leu Arg Pro
                                165 170 175
Asp Glu His Ile Ala Lys Gln Arg Tyr Ile Gly Gly Lys Asn Leu Pro
                        180 185 190
Thr Arg Gly Thr Ala Glu Trp Leu Ser Phe Asp Val Thr Asp Thr Val
                195 200 205
Arg Glu Trp Leu Leu Arg Arg Glu Ser Asn Leu Gly Leu Glu Ile Ser
        210 215 220
Ile His Cys Pro Cys His Thr Phe Gln Pro Asn Gly Asp Ile Leu Glu
225 230 235 240
Asn Ile His Glu Val Met Glu Ile Lys Phe Lys Gly Val Asp Asn Glu
                                245 250 255
Asp Asp His Gly Arg Gly Asp Leu Gly Arg Leu Lys Lys Gln Lys Asp
                        260 265 270
His His Asn Pro His Leu Ile Leu Met Met Ile Pro Pro His Arg Leu
                275 280 285
Asp Asn Pro Gly Gln Gly Gly Gln Arg Lys Lys Arg Ala Leu Asp Thr
        290 295 300
Asn Tyr Cys Phe Arg Asn Leu Glu Glu Asn Cys Cys Val Arg Pro Leu
305 310 315 320
Tyr Ile Asp Phe Arg Gln Asp Leu Gly Trp Lys Trp Val His Glu Pro
                                325 330 335
Lys Gly Tyr Tyr Ala Asn Phe Cys Ser Gly Pro Cys Pro Tyr Leu Arg
                        340 345 350
Ser Ala Asp Thr Thr His Ser Thr Val Leu Gly Leu Tyr Asn Thr Leu
                355 360 365
Asn Pro Glu Ala Ser Ala Ser Pro Cys Cys Val Pro Gln Asp Leu Glu
        370 375 380
Pro Leu Thr Ile Leu Tyr Tyr Val Gly Arg Thr Pro Lys Val Glu Gln
385 390 395 400
Leu Ser Asn Met Val Val Lys Ser Cys Lys Cys Ser
                                405 410
<210> SEQ ID NO 125
<211> LENGTH: 155
<212> TYPE: PRT
<213> ORGANISM: Homo sapiens
<400> SEQUENCE: 125
Met Ala Glu Gly Glu Ile Thr Thr Phe Thr Ala Leu Thr Glu Lys Phe
1 5 10 15
Asn Leu Pro Pro Gly Asn Tyr Lys Lys Pro Lys Leu Leu Tyr Cys Ser
                        20 25 30
Asn Gly Gly His Phe Leu Arg Ile Leu Pro Asp Gly Thr Val Asp Gly
                35 40 45
Thr Arg Asp Arg Ser Asp Gln His Ile Gln Leu Gln Leu Ser Ala Glu
        50 55 60
Ser Val Gly Glu Val Tyr Ile Lys Ser Thr Glu Thr Gly Gln Tyr Leu
65 70 75 80
Ala Met Asp Thr Asp Gly Leu Leu Tyr Gly Ser Gln Thr Pro Asn Glu
                                85 90 95
Glu Cys Leu Phe Leu Glu Arg Leu Glu Glu Asn His Tyr Asn Thr Tyr
                        100 105 110
Ile Ser Lys Lys His Ala Glu Lys Asn Trp Phe Val Gly Leu Lys Lys
                115 120 125
Asn Gly Ser Cys Lys Arg Gly Pro Arg Thr His Tyr Gly Gln Lys Ala
        130 135 140
Ile Leu Phe Leu Pro Leu Pro Val Ser Ser Asp
145 150 155
<210> SEQ ID NO 126
<211> LENGTH: 60
<212> TYPE: PRT
<213> ORGANISM: Homo sapiens
<400> SEQUENCE: 126
Met Ala Glu Gly Glu Ile Thr Thr Phe Thr Ala Leu Thr Glu Lys Phe
1 5 10 15
Asn Leu Pro Pro Gly Asn Tyr Lys Lys Pro Lys Leu Leu Tyr Cys Ser
                        20 25 30
Asn Gly Gly His Phe Leu Arg Ile Leu Pro Asp Gly Thr Val Asp Gly
                35 40 45
Thr Arg Asp Arg Ser Asp Gln His Thr Asp Thr Lys
        50 55 60
<210> SEQ ID NO 127
<211> LENGTH: 59
<212> TYPE: PRT
<213> ORGANISM: Homo sapiens
<400> SEQUENCE: 127
Met Ala Glu Gly Glu Ile Thr Thr Phe Thr Ala Leu Thr Glu Lys Phe
1 5 10 15
Asn Leu Pro Pro Gly Asn Tyr Lys Lys Pro Lys Leu Leu Tyr Cys Ser
                        20 25 30
Asn Gly Gly His Phe Leu Arg Ile Leu Pro Asp Gly Thr Val Asp Gly
                35 40 45
Thr Arg Asp Arg Ser Asp Gln His Asn Thr Lys
        50 55
<210> SEQ ID NO 128
<211> LENGTH: 155
<212> TYPE: PRT
<213> ORGANISM: Homo sapiens
<400> SEQUENCE: 128
Met Ala Glu Gly Glu Ile Thr Thr Phe Thr Ala Leu Thr Glu Lys Phe
1 5 10 15
Asn Leu Pro Pro Gly Asn Tyr Lys Lys Pro Lys Leu Leu Tyr Cys Ser
                        20 25 30
Asn Gly Gly His Phe Leu Arg Ile Leu Pro Asp Gly Thr Val Asp Gly
                35 40 45
Thr Arg Asp Arg Ser Asp Gln His Ile Gln Leu Gln Leu Ser Ala Glu
        50 55 60
Ser Val Gly Glu Val Tyr Ile Lys Ser Thr Glu Thr Gly Gln Tyr Leu
65 70 75 80
Ala Met Asp Thr Asp Gly Leu Leu Tyr Gly Ser Gln Thr Pro Asn Glu
                                85 90 95
Glu Cys Leu Phe Leu Glu Arg Leu Glu Glu Asn His Tyr Asn Thr Tyr
                        100 105 110
Ile Ser Lys Lys His Ala Glu Lys Asn Trp Phe Val Gly Leu Lys Lys
                115 120 125
Asn Gly Ser Cys Lys Arg Gly Pro Arg Thr His Tyr Gly Gln Lys Ala
        130 135 140
Ile Leu Phe Leu Pro Leu Pro Val Ser Ser Asp
145 150 155
<210> SEQ ID NO 129
<211> LENGTH: 155
<212> TYPE: PRT
<213> ORGANISM: Homo sapiens
<400> SEQUENCE: 129
Met Ala Glu Gly Glu Ile Thr Thr Phe Thr Ala Leu Thr Glu Lys Phe
1 5 10 15
Asn Leu Pro Pro Gly Asn Tyr Lys Lys Pro Lys Leu Leu Tyr Cys Ser
                        20 25 30
Asn Gly Gly His Phe Leu Arg Ile Leu Pro Asp Gly Thr Val Asp Gly
                35 40 45
Thr Arg Asp Arg Ser Asp Gln His Ile Gln Leu Gln Leu Ser Ala Glu
        50 55 60
Ser Val Gly Glu Val Tyr Ile Lys Ser Thr Glu Thr Gly Gln Tyr Leu
65 70 75 80
Ala Met Asp Thr Asp Gly Leu Leu Tyr Gly Ser Gln Thr Pro Asn Glu
                                85 90 95
Glu Cys Leu Phe Leu Glu Arg Leu Glu Glu Asn His Tyr Asn Thr Tyr
                        100 105 110
Ile Ser Lys Lys His Ala Glu Lys Asn Trp Phe Val Gly Leu Lys Lys
                115 120 125
Asn Gly Ser Cys Lys Arg Gly Pro Arg Thr His Tyr Gly Gln Lys Ala
        130 135 140
Ile Leu Phe Leu Pro Leu Pro Val Ser Ser Asp
145 150 155
<210> SEQ ID NO 130
<211> LENGTH: 155
<212> TYPE: PRT
<213> ORGANISM: Homo sapiens
<400> SEQUENCE: 130
Met Ala Glu Gly Glu Ile Thr Thr Phe Thr Ala Leu Thr Glu Lys Phe
1 5 10 15
Asn Leu Pro Pro Gly Asn Tyr Lys Lys Pro Lys Leu Leu Tyr Cys Ser
                        20 25 30
Asn Gly Gly His Phe Leu Arg Ile Leu Pro Asp Gly Thr Val Asp Gly
                35 40 45
Thr Arg Asp Arg Ser Asp Gln His Ile Gln Leu Gln Leu Ser Ala Glu
        50 55 60
Ser Val Gly Glu Val Tyr Ile Lys Ser Thr Glu Thr Gly Gln Tyr Leu
65 70 75 80
Ala Met Asp Thr Asp Gly Leu Leu Tyr Gly Ser Gln Thr Pro Asn Glu
                                85 90 95
Glu Cys Leu Phe Leu Glu Arg Leu Glu Glu Asn His Tyr Asn Thr Tyr
                        100 105 110
Ile Ser Lys Lys His Ala Glu Lys Asn Trp Phe Val Gly Leu Lys Lys
                115 120 125
Asn Gly Ser Cys Lys Arg Gly Pro Arg Thr His Tyr Gly Gln Lys Ala
        130 135 140
Ile Leu Phe Leu Pro Leu Pro Val Ser Ser Asp
145 150 155
<210> SEQ ID NO 131
<211> LENGTH: 155
<212> TYPE: PRT
<213> ORGANISM: Homo sapiens
<400> SEQUENCE: 131
Met Ala Glu Gly Glu Ile Thr Thr Phe Thr Ala Leu Thr Glu Lys Phe
1 5 10 15
Asn Leu Pro Pro Gly Asn Tyr Lys Lys Pro Lys Leu Leu Tyr Cys Ser
                        20 25 30
Asn Gly Gly His Phe Leu Arg Ile Leu Pro Asp Gly Thr Val Asp Gly
                35 40 45
Thr Arg Asp Arg Ser Asp Gln His Ile Gln Leu Gln Leu Ser Ala Glu
        50 55 60
Ser Val Gly Glu Val Tyr Ile Lys Ser Thr Glu Thr Gly Gln Tyr Leu
65 70 75 80
Ala Met Asp Thr Asp Gly Leu Leu Tyr Gly Ser Gln Thr Pro Asn Glu
                                85 90 95
Glu Cys Leu Phe Leu Glu Arg Leu Glu Glu Asn His Tyr Asn Thr Tyr
                        100 105 110
Ile Ser Lys Lys His Ala Glu Lys Asn Trp Phe Val Gly Leu Lys Lys
                115 120 125
Asn Gly Ser Cys Lys Arg Gly Pro Arg Thr His Tyr Gly Gln Lys Ala
        130 135 140
Ile Leu Phe Leu Pro Leu Pro Val Ser Ser Asp
145 150 155
<210> SEQ ID NO 132
<211> LENGTH: 154
<212> TYPE: PRT
<213> ORGANISM: Homo sapiens
<400> SEQUENCE: 132
Met Ala Glu Gly Glu Ile Thr Thr Phe Thr Ala Leu Thr Glu Lys Phe
1 5 10 15
Asn Leu Pro Pro Gly Asn Tyr Lys Lys Pro Lys Leu Leu Tyr Cys Ser
                        20 25 30
Asn Gly Gly His Phe Leu Arg Ile Leu Pro Asp Gly Thr Val Asp Gly
                35 40 45
Thr Arg Asp Arg Ser Asp Gln His Ile Gln Leu Gln Leu Ser Ala Glu
        50 55 60
Ser Val Gly Glu Val Tyr Ile Lys Ser Thr Glu Thr Gly Gln Tyr Leu
65 70 75 80
Ala Met Asp Thr Asp Gly Leu Leu Tyr Gly Ser Thr Pro Asn Glu Glu
                                85 90 95
Cys Leu Phe Leu Glu Arg Leu Glu Glu Asn His Tyr Asn Thr Tyr Ile
                        100 105 110
Ser Lys Lys His Ala Glu Lys Asn Trp Phe Val Gly Leu Lys Lys Asn
                115 120 125
Gly Ser Cys Lys Arg Gly Pro Arg Thr His Tyr Gly Gln Lys Ala Ile
        130 135 140
Leu Phe Leu Pro Leu Pro Val Ser Ser Asp
145 150
<210> SEQ ID NO 133
<211> LENGTH: 155
<212> TYPE: PRT
<213> ORGANISM: Homo sapiens
<400> SEQUENCE: 133
Met Ala Glu Gly Glu Ile Thr Thr Phe Thr Ala Leu Thr Glu Lys Phe
1 5 10 15
Asn Leu Pro Pro Gly Asn Tyr Lys Lys Pro Lys Leu Leu Tyr Cys Ser
                        20 25 30
Asn Gly Gly His Phe Leu Arg Ile Leu Pro Asp Gly Thr Val Asp Gly
                35 40 45
Thr Arg Asp Arg Ser Asp Gln His Ile Gln Leu Gln Leu Ser Ala Glu
        50 55 60
Ser Val Gly Glu Val Tyr Ile Lys Ser Thr Glu Thr Gly Gln Tyr Leu
65 70 75 80
Ala Met Asp Thr Asp Gly Leu Leu Tyr Gly Ser Gln Thr Pro Asn Glu
                                85 90 95
Glu Cys Leu Phe Leu Glu Arg Leu Glu Glu Asn His Tyr Asn Thr Tyr
                        100 105 110
Ile Ser Lys Lys His Ala Glu Lys Asn Trp Phe Val Gly Leu Lys Lys
                115 120 125
Asn Gly Ser Cys Lys Arg Gly Pro Arg Thr His Tyr Gly Gln Lys Ala
        130 135 140
Ile Leu Phe Leu Pro Leu Pro Val Ser Ser Asp
145 150 155
<210> SEQ ID NO 134
<211> LENGTH: 155
<212> TYPE: PRT
<213> ORGANISM: Homo sapiens
<400> SEQUENCE: 134
Met Ala Glu Gly Glu Ile Thr Thr Phe Thr Ala Leu Thr Glu Lys Phe
1 5 10 15
Asn Leu Pro Pro Gly Asn Tyr Lys Lys Pro Lys Leu Leu Tyr Cys Ser
                        20 25 30
Asn Gly Gly His Phe Leu Arg Ile Leu Pro Asp Gly Thr Val Asp Gly
                35 40 45
Thr Arg Asp Arg Ser Asp Gln His Ile Gln Leu Gln Leu Ser Ala Glu
        50 55 60
Ser Val Gly Glu Val Tyr Ile Lys Ser Thr Glu Thr Gly Gln Tyr Leu
65 70 75 80
Ala Met Asp Thr Asp Gly Leu Leu Tyr Gly Ser Gln Thr Pro Asn Glu
                                85 90 95
Glu Cys Leu Phe Leu Glu Arg Leu Glu Glu Asn His Tyr Asn Thr Tyr
                        100 105 110
Ile Ser Lys Lys His Ala Glu Lys Asn Trp Phe Val Gly Leu Lys Lys
                115 120 125
Asn Gly Ser Cys Lys Arg Gly Pro Arg Thr His Tyr Gly Gln Lys Ala
        130 135 140
Ile Leu Phe Leu Pro Leu Pro Val Ser Ser Asp
145 150 155
<210> SEQ ID NO 135
<211> LENGTH: 155
<212> TYPE: PRT
<213> ORGANISM: Homo sapiens
<400> SEQUENCE: 135
Met Ala Glu Gly Glu Ile Thr Thr Phe Thr Ala Leu Thr Glu Lys Phe
1 5 10 15
Asn Leu Pro Pro Gly Asn Tyr Lys Lys Pro Lys Leu Leu Tyr Cys Ser
                        20 25 30
Asn Gly Gly His Phe Leu Arg Ile Leu Pro Asp Gly Thr Val Asp Gly
                35 40 45
Thr Arg Asp Arg Ser Asp Gln His Ile Gln Leu Gln Leu Ser Ala Glu
        50 55 60
Ser Val Gly Glu Val Tyr Ile Lys Ser Thr Glu Thr Gly Gln Tyr Leu
65 70 75 80
Ala Met Asp Thr Asp Gly Leu Leu Tyr Gly Ser Gln Thr Pro Asn Glu
                                85 90 95
Glu Cys Leu Phe Leu Glu Arg Leu Glu Glu Asn His Tyr Asn Thr Tyr
                        100 105 110
Ile Ser Lys Lys His Ala Glu Lys Asn Trp Phe Val Gly Leu Lys Lys
                115 120 125
Asn Gly Ser Cys Lys Arg Gly Pro Arg Thr His Tyr Gly Gln Lys Ala
        130 135 140
Ile Leu Phe Leu Pro Leu Pro Val Ser Ser Asp
145 150 155
<210> SEQ ID NO 136
<211> LENGTH: 155
<212> TYPE: PRT
<213> ORGANISM: Homo sapiens
<400> SEQUENCE: 136
Met Ala Glu Gly Glu Ile Thr Thr Phe Thr Ala Leu Thr Glu Lys Phe
1 5 10 15
Asn Leu Pro Pro Gly Asn Tyr Lys Lys Pro Lys Leu Leu Tyr Cys Ser
                        20 25 30
Asn Gly Gly His Phe Leu Arg Ile Leu Pro Asp Gly Thr Val Asp Gly
                35 40 45
Thr Arg Asp Arg Ser Asp Gln His Ile Gln Leu Gln Leu Ser Ala Glu
        50 55 60
Ser Val Gly Glu Val Tyr Ile Lys Ser Thr Glu Thr Gly Gln Tyr Leu
65 70 75 80
Ala Met Asp Thr Asp Gly Leu Leu Tyr Gly Ser Gln Thr Pro Asn Glu
                                85 90 95
Glu Cys Leu Phe Leu Glu Arg Leu Glu Glu Asn His Tyr Asn Thr Tyr
                        100 105 110
Ile Ser Lys Lys His Ala Glu Lys Asn Trp Phe Val Gly Leu Lys Lys
                115 120 125
Asn Gly Ser Cys Lys Arg Gly Pro Arg Thr His Tyr Gly Gln Lys Ala
        130 135 140
Ile Leu Phe Leu Pro Leu Pro Val Ser Ser Asp
145 150 155
<210> SEQ ID NO 137
<211> LENGTH: 154
<212> TYPE: PRT
<213> ORGANISM: Homo sapiens
<400> SEQUENCE: 137
Met Ala Glu Gly Glu Ile Thr Thr Phe Thr Ala Leu Thr Glu Lys Phe
1 5 10 15
Asn Leu Pro Pro Gly Asn Tyr Lys Lys Pro Lys Leu Leu Tyr Cys Ser
                        20 25 30
Asn Gly Gly His Phe Leu Arg Ile Leu Pro Asp Gly Thr Val Asp Gly
                35 40 45
Thr Arg Asp Arg Ser Asp Gln His Ile Gln Leu Gln Leu Ser Ala Glu
        50 55 60
Ser Val Gly Glu Val Tyr Ile Lys Ser Thr Glu Thr Gly Gln Tyr Leu
65 70 75 80
Ala Met Asp Thr Asp Gly Leu Leu Tyr Gly Ser Thr Pro Asn Glu Glu
                                85 90 95
Cys Leu Phe Leu Glu Arg Leu Glu Glu Asn His Tyr Asn Thr Tyr Ile
                        100 105 110
Ser Lys Lys His Ala Glu Lys Asn Trp Phe Val Gly Leu Lys Lys Asn
                115 120 125
Gly Ser Cys Lys Arg Gly Pro Arg Thr His Tyr Gly Gln Lys Ala Ile
        130 135 140
Leu Phe Leu Pro Leu Pro Val Ser Ser Asp
145 150
<210> SEQ ID NO 138
<211> LENGTH: 154
<212> TYPE: PRT
<213> ORGANISM: Homo sapiens
<400> SEQUENCE: 138
Met Ala Glu Gly Glu Ile Thr Thr Phe Thr Ala Leu Thr Glu Lys Phe
1 5 10 15
Asn Leu Pro Pro Gly Asn Tyr Lys Lys Pro Lys Leu Leu Tyr Cys Ser
                        20 25 30
Asn Gly Gly His Phe Leu Arg Ile Leu Pro Asp Gly Thr Val Asp Gly
                35 40 45
Thr Arg Asp Arg Ser Asp Gln His Ile Gln Leu Gln Leu Ser Ala Glu
        50 55 60
Ser Val Gly Glu Val Tyr Ile Lys Ser Thr Glu Thr Gly Gln Tyr Leu
65 70 75 80
Ala Met Asp Thr Asp Gly Leu Leu Tyr Gly Ser Thr Pro Asn Glu Glu
                                85 90 95
Cys Leu Phe Leu Glu Arg Leu Glu Glu Asn His Tyr Asn Thr Tyr Ile
                        100 105 110
Ser Lys Lys His Ala Glu Lys Asn Trp Phe Val Gly Leu Lys Lys Asn
                115 120 125
Gly Ser Cys Lys Arg Gly Pro Arg Thr His Tyr Gly Gln Lys Ala Ile
        130 135 140
Leu Phe Leu Pro Leu Pro Val Ser Ser Asp
145 150
<210> SEQ ID NO 139
<211> LENGTH: 288
<212> TYPE: PRT
<213> ORGANISM: Homo sapiens
<400> SEQUENCE: 139
Met Val Gly Val Gly Gly Gly Asp Val Glu Asp Val Thr Pro Arg Pro
1 5 10 15
Gly Gly Cys Gln Ile Ser Gly Arg Gly Ala Arg Gly Cys Asn Gly Ile
                        20 25 30
Pro Gly Ala Ala Ala Trp Glu Ala Ala Leu Pro Arg Arg Arg Pro Arg
                35 40 45
Arg His Pro Ser Val Asn Pro Arg Ser Arg Ala Ala Gly Ser Pro Arg
        50 55 60
Thr Arg Gly Arg Arg Thr Glu Glu Arg Pro Ser Gly Ser Arg Leu Gly
65 70 75 80
Asp Arg Gly Arg Gly Arg Ala Leu Pro Gly Gly Arg Leu Gly Gly Arg
                                85 90 95
Gly Arg Gly Arg Ala Pro Glu Arg Val Gly Gly Arg Gly Arg Gly Arg
                        100 105 110
Gly Thr Ala Ala Pro Arg Ala Ala Pro Ala Ala Arg Gly Ser Arg Pro
                115 120 125
Gly Pro Ala Gly Thr Met Ala Ala Gly Ser Ile Thr Thr Leu Pro Ala
        130 135 140
Leu Pro Glu Asp Gly Gly Ser Gly Ala Phe Pro Pro Gly His Phe Lys
145 150 155 160
Asp Pro Lys Arg Leu Tyr Cys Lys Asn Gly Gly Phe Phe Leu Arg Ile
                                165 170 175
His Pro Asp Gly Arg Val Asp Gly Val Arg Glu Lys Ser Asp Pro His
                        180 185 190
Ile Lys Leu Gln Leu Gln Ala Glu Glu Arg Gly Val Val Ser Ile Lys
                195 200 205
Gly Val Cys Ala Asn Arg Tyr Leu Ala Met Lys Glu Asp Gly Arg Leu
        210 215 220
Leu Ala Ser Lys Cys Val Thr Asp Glu Cys Phe Phe Phe Glu Arg Leu
225 230 235 240
Glu Ser Asn Asn Tyr Asn Thr Tyr Arg Ser Arg Lys Tyr Thr Ser Trp
                                245 250 255
Tyr Val Ala Leu Lys Arg Thr Gly Gln Tyr Lys Leu Gly Ser Lys Thr
                        260 265 270
Gly Pro Gly Gln Lys Ala Ile Leu Phe Leu Pro Met Ser Ala Lys Ser
                275 280 285
<210> SEQ ID NO 140
<211> LENGTH: 239
<212> TYPE: PRT
<213> ORGANISM: Homo sapiens
<400> SEQUENCE: 140
Met Gly Leu Ile Trp Leu Leu Leu Leu Ser Leu Leu Glu Pro Gly Trp
1 5 10 15
Pro Ala Ala Gly Pro Gly Ala Arg Leu Arg Arg Asp Ala Gly Gly Arg
                        20 25 30
Gly Gly Val Tyr Glu His Leu Gly Gly Ala Pro Arg Arg Arg Lys Leu
                35 40 45
Tyr Cys Ala Thr Lys Tyr His Leu Gln Leu His Pro Ser Gly Arg Val
        50 55 60
Asn Gly Ser Leu Glu Asn Ser Ala Tyr Ser Ile Leu Glu Ile Thr Ala
65 70 75 80
Val Glu Val Gly Ile Val Ala Ile Arg Gly Leu Phe Ser Gly Arg Tyr
                                85 90 95
Leu Ala Met Asn Lys Arg Gly Arg Leu Tyr Ala Ser Glu His Tyr Ser
                        100 105 110
Ala Glu Cys Glu Phe Val Glu Arg Ile His Glu Leu Gly Tyr Asn Thr
                115 120 125
Tyr Ala Ser Arg Leu Tyr Arg Thr Val Ser Ser Thr Pro Gly Ala Arg
        130 135 140
Arg Gln Pro Ser Ala Glu Arg Leu Trp Tyr Val Ser Val Asn Gly Lys
145 150 155 160
Gly Arg Pro Arg Arg Gly Phe Lys Thr Arg Arg Thr Gln Lys Ser Ser
                                165 170 175
Leu Phe Leu Pro Arg Val Leu Asp His Arg Asp His Glu Met Val Arg
                        180 185 190
Gln Leu Gln Ser Gly Leu Pro Arg Pro Pro Gly Lys Gly Val Gln Pro
                195 200 205
Arg Arg Arg Arg Gln Lys Gln Ser Pro Asp Asn Leu Glu Pro Ser His
        210 215 220
Val Gln Ala Ser Arg Leu Gly Ser Gln Leu Glu Ala Ser Ala His
225 230 235
<210> SEQ ID NO 141
<211> LENGTH: 206
<212> TYPE: PRT
<213> ORGANISM: Homo sapiens
<400> SEQUENCE: 141
Met Ser Gly Pro Gly Thr Ala Ala Val Ala Leu Leu Pro Ala Val Leu
1 5 10 15
Leu Ala Leu Leu Ala Pro Trp Ala Gly Arg Gly Gly Ala Ala Ala Pro
                        20 25 30
Thr Ala Pro Asn Gly Thr Leu Glu Ala Glu Leu Glu Arg Arg Trp Glu
                35 40 45
Ser Leu Val Ala Leu Ser Leu Ala Arg Leu Pro Val Ala Ala Gln Pro
        50 55 60
Lys Glu Ala Ala Val Gln Ser Gly Ala Gly Asp Tyr Leu Leu Gly Ile
65 70 75 80
Lys Arg Leu Arg Arg Leu Tyr Cys Asn Val Gly Ile Gly Phe His Leu
                                85 90 95
Gln Ala Leu Pro Asp Gly Arg Ile Gly Gly Ala His Ala Asp Thr Arg
                        100 105 110
Asp Ser Leu Leu Glu Leu Ser Pro Val Glu Arg Gly Val Val Ser Ile
                115 120 125
Phe Gly Val Ala Ser Arg Phe Phe Val Ala Met Ser Ser Lys Gly Lys
        130 135 140
Leu Tyr Gly Ser Pro Phe Phe Thr Asp Glu Cys Thr Phe Lys Glu Ile
145 150 155 160
Leu Leu Pro Asn Asn Tyr Asn Ala Tyr Glu Ser Tyr Lys Tyr Pro Gly
                                165 170 175
Met Phe Ile Ala Leu Ser Lys Asn Gly Lys Thr Lys Lys Gly Asn Arg
                        180 185 190
Val Ser Pro Thr Met Lys Val Thr His Phe Leu Pro Arg Leu
                195 200 205
<210> SEQ ID NO 142
<211> LENGTH: 268
<212> TYPE: PRT
<213> ORGANISM: Homo sapiens
<400> SEQUENCE: 142
Met Ser Leu Ser Phe Leu Leu Leu Leu Phe Phe Ser His Leu Ile Leu
1 5 10 15
Ser Ala Trp Ala His Gly Glu Lys Arg Leu Ala Pro Lys Gly Gln Pro
                        20 25 30
Gly Pro Ala Ala Thr Asp Arg Asn Pro Arg Gly Ser Ser Ser Arg Gln
                35 40 45
Ser Ser Ser Ser Ala Met Ser Ser Ser Ser Ala Ser Ser Ser Pro Ala
        50 55 60
Ala Ser Leu Gly Ser Gln Gly Ser Gly Leu Glu Gln Ser Ser Phe Gln
65 70 75 80
Trp Ser Pro Ser Gly Arg Arg Thr Gly Ser Leu Tyr Cys Arg Val Gly
                                85 90 95
Ile Gly Phe His Leu Gln Ile Tyr Pro Asp Gly Lys Val Asn Gly Ser
                        100 105 110
His Glu Ala Asn Met Leu Ser Val Leu Glu Ile Phe Ala Val Ser Gln
                115 120 125
Gly Ile Val Gly Ile Arg Gly Val Phe Ser Asn Lys Phe Leu Ala Met
        130 135 140
Ser Lys Lys Gly Lys Leu His Ala Ser Ala Lys Phe Thr Asp Asp Cys
145 150 155 160
Lys Phe Arg Glu Arg Phe Gln Glu Asn Ser Tyr Asn Thr Tyr Ala Ser
                                165 170 175
Ala Ile His Arg Thr Glu Lys Thr Gly Arg Glu Trp Tyr Val Ala Leu
                        180 185 190
Asn Lys Arg Gly Lys Ala Lys Arg Gly Cys Ser Pro Arg Val Lys Pro
                195 200 205
Gln His Ile Ser Thr His Phe Leu Pro Arg Phe Lys Gln Ser Glu Gln
        210 215 220
Pro Glu Leu Ser Phe Thr Val Thr Val Pro Glu Lys Lys Lys Pro Pro
225 230 235 240
Ser Pro Ile Lys Pro Lys Ile Pro Leu Ser Ala Pro Arg Lys Asn Thr
                                245 250 255
Asn Ser Val Lys Tyr Arg Leu Lys Phe Arg Phe Gly
                        260 265
<210> SEQ ID NO 143
<211> LENGTH: 123
<212> TYPE: PRT
<213> ORGANISM: Homo sapiens
<400> SEQUENCE: 143
Met Ser Leu Ser Phe Leu Leu Leu Leu Phe Phe Ser His Leu Ile Leu
1 5 10 15
Ser Ala Trp Ala His Gly Glu Lys Arg Leu Ala Pro Lys Gly Gln Pro
                        20 25 30
Gly Pro Ala Ala Thr Asp Arg Asn Pro Arg Gly Ser Ser Ser Arg Gln
                35 40 45
Ser Ser Ser Ser Ala Met Ser Ser Ser Ser Ala Ser Ser Ser Pro Ala
        50 55 60
Ala Ser Leu Gly Ser Gln Gly Ser Gly Leu Glu Gln Ser Ser Phe Gln
65 70 75 80
Trp Ser Pro Ser Gly Arg Arg Thr Gly Ser Leu Tyr Cys Arg Val Gly
                                85 90 95
Ile Gly Phe His Leu Gln Ile Tyr Pro Asp Gly Lys Val Asn Gly Ser
                        100 105 110
His Glu Ala Asn Met Leu Ser Gln Val His Arg
                115 120
<210> SEQ ID NO 144
<211> LENGTH: 208
<212> TYPE: PRT
<213> ORGANISM: Homo sapiens
<400> SEQUENCE: 144
Met Ala Leu Gly Gln Lys Leu Phe Ile Thr Met Ser Arg Gly Ala Gly
1 5 10 15
Arg Leu Gln Gly Thr Leu Trp Ala Leu Val Phe Leu Gly Ile Leu Val
                        20 25 30
Gly Met Val Val Pro Ser Pro Ala Gly Thr Arg Ala Asn Asn Thr Leu
                35 40 45
Leu Asp Ser Arg Gly Trp Gly Thr Leu Leu Ser Arg Ser Arg Ala Gly
        50 55 60
Leu Ala Gly Glu Ile Ala Gly Val Asn Trp Glu Ser Gly Tyr Leu Val
65 70 75 80
Gly Ile Lys Arg Gln Arg Arg Leu Tyr Cys Asn Val Gly Ile Gly Phe
                                85 90 95
His Leu Gln Val Leu Pro Asp Gly Arg Ile Ser Gly Thr His Glu Glu
                        100 105 110
Asn Pro Tyr Ser Leu Leu Glu Ile Ser Thr Val Glu Arg Gly Val Val
                115 120 125
Ser Leu Phe Gly Val Arg Ser Ala Leu Phe Val Ala Met Asn Ser Lys
        130 135 140
Gly Arg Leu Tyr Ala Thr Pro Ser Phe Gln Glu Glu Cys Lys Phe Arg
145 150 155 160
Glu Thr Leu Leu Pro Asn Asn Tyr Asn Ala Tyr Glu Ser Asp Leu Tyr
                                165 170 175
Gln Gly Thr Tyr Ile Ala Leu Ser Lys Tyr Gly Arg Val Lys Arg Gly
                        180 185 190
Ser Lys Val Ser Pro Ile Met Thr Val Thr His Phe Leu Pro Arg Ile
                195 200 205
<210> SEQ ID NO 145
<211> LENGTH: 204
<212> TYPE: PRT
<213> ORGANISM: Homo sapiens
<400> SEQUENCE: 145
Met Gly Ser Pro Arg Ser Ala Leu Ser Cys Leu Leu Leu His Leu Leu
1 5 10 15
Val Leu Cys Leu Gln Ala Gln His Val Arg Glu Gln Ser Leu Val Thr
                        20 25 30
Asp Gln Leu Ser Arg Arg Leu Ile Arg Thr Tyr Gln Leu Tyr Ser Arg
                35 40 45
Thr Ser Gly Lys His Val Gln Val Leu Ala Asn Lys Arg Ile Asn Ala
        50 55 60
Met Ala Glu Asp Gly Asp Pro Phe Ala Lys Leu Ile Val Glu Thr Asp
65 70 75 80
Thr Phe Gly Ser Arg Val Arg Val Arg Gly Ala Glu Thr Gly Leu Tyr
                                85 90 95
Ile Cys Met Asn Lys Lys Gly Lys Leu Ile Ala Lys Ser Asn Gly Lys
                        100 105 110
Gly Lys Asp Cys Val Phe Thr Glu Ile Val Leu Glu Asn Asn Tyr Thr
                115 120 125
Ala Leu Gln Asn Ala Lys Tyr Glu Gly Trp Tyr Met Ala Phe Thr Arg
        130 135 140
Lys Gly Arg Pro Arg Lys Gly Ser Lys Thr Arg Gln His Gln Arg Glu
145 150 155 160
Val His Phe Met Lys Arg Leu Pro Arg Gly His His Thr Thr Glu Gln
                                165 170 175
Ser Leu Arg Phe Glu Phe Leu Asn Tyr Pro Pro Phe Thr Arg Ser Leu
                        180 185 190
Arg Gly Ser Gln Arg Thr Trp Ala Pro Glu Pro Arg
                195 200
<210> SEQ ID NO 146
<211> LENGTH: 215
<212> TYPE: PRT
<213> ORGANISM: Homo sapiens
<400> SEQUENCE: 146
Met Gly Ser Pro Arg Ser Ala Leu Ser Cys Leu Leu Leu His Leu Leu
1 5 10 15
Val Leu Cys Leu Gln Ala Gln Val Thr Val Gln Ser Ser Pro Asn Phe
                        20 25 30
Thr Gln His Val Arg Glu Gln Ser Leu Val Thr Asp Gln Leu Ser Arg
                35 40 45
Arg Leu Ile Arg Thr Tyr Gln Leu Tyr Ser Arg Thr Ser Gly Lys His
        50 55 60
Val Gln Val Leu Ala Asn Lys Arg Ile Asn Ala Met Ala Glu Asp Gly
65 70 75 80
Asp Pro Phe Ala Lys Leu Ile Val Glu Thr Asp Thr Phe Gly Ser Arg
                                85 90 95
Val Arg Val Arg Gly Ala Glu Thr Gly Leu Tyr Ile Cys Met Asn Lys
                        100 105 110
Lys Gly Lys Leu Ile Ala Lys Ser Asn Gly Lys Gly Lys Asp Cys Val
                115 120 125
Phe Thr Glu Ile Val Leu Glu Asn Asn Tyr Thr Ala Leu Gln Asn Ala
        130 135 140
Lys Tyr Glu Gly Trp Tyr Met Ala Phe Thr Arg Lys Gly Arg Pro Arg
145 150 155 160
Lys Gly Ser Lys Thr Arg Gln His Gln Arg Glu Val His Phe Met Lys
                                165 170 175
Arg Leu Pro Arg Gly His His Thr Thr Glu Gln Ser Leu Arg Phe Glu
                        180 185 190
Phe Leu Asn Tyr Pro Pro Phe Thr Arg Ser Leu Arg Gly Ser Gln Arg
                195 200 205
Thr Trp Ala Pro Glu Pro Arg
        210 215
<210> SEQ ID NO 147
<211> LENGTH: 233
<212> TYPE: PRT
<213> ORGANISM: Homo sapiens
<400> SEQUENCE: 147
Met Gly Ser Pro Arg Ser Ala Leu Ser Cys Leu Leu Leu His Leu Leu
1 5 10 15
Val Leu Cys Leu Gln Ala Gln Glu Gly Pro Gly Arg Gly Pro Ala Leu
                        20 25 30
Gly Arg Glu Leu Ala Ser Leu Phe Arg Ala Gly Arg Glu Pro Gln Gly
                35 40 45
Val Ser Gln Gln His Val Arg Glu Gln Ser Leu Val Thr Asp Gln Leu
        50 55 60
Ser Arg Arg Leu Ile Arg Thr Tyr Gln Leu Tyr Ser Arg Thr Ser Gly
65 70 75 80
Lys His Val Gln Val Leu Ala Asn Lys Arg Ile Asn Ala Met Ala Glu
                                85 90 95
Asp Gly Asp Pro Phe Ala Lys Leu Ile Val Glu Thr Asp Thr Phe Gly
                        100 105 110
Ser Arg Val Arg Val Arg Gly Ala Glu Thr Gly Leu Tyr Ile Cys Met
                115 120 125
Asn Lys Lys Gly Lys Leu Ile Ala Lys Ser Asn Gly Lys Gly Lys Asp
        130 135 140
Cys Val Phe Thr Glu Ile Val Leu Glu Asn Asn Tyr Thr Ala Leu Gln
145 150 155 160
Asn Ala Lys Tyr Glu Gly Trp Tyr Met Ala Phe Thr Arg Lys Gly Arg
                                165 170 175
Pro Arg Lys Gly Ser Lys Thr Arg Gln His Gln Arg Glu Val His Phe
                        180 185 190
Met Lys Arg Leu Pro Arg Gly His His Thr Thr Glu Gln Ser Leu Arg
                195 200 205
Phe Glu Phe Leu Asn Tyr Pro Pro Phe Thr Arg Ser Leu Arg Gly Ser
        210 215 220
Gln Arg Thr Trp Ala Pro Glu Pro Arg
225 230
<210> SEQ ID NO 148
<211> LENGTH: 244
<212> TYPE: PRT
<213> ORGANISM: Homo sapiens
<400> SEQUENCE: 148
Met Gly Ser Pro Arg Ser Ala Leu Ser Cys Leu Leu Leu His Leu Leu
1 5 10 15
Val Leu Cys Leu Gln Ala Gln Glu Gly Pro Gly Arg Gly Pro Ala Leu
                        20 25 30
Gly Arg Glu Leu Ala Ser Leu Phe Arg Ala Gly Arg Glu Pro Gln Gly
                35 40 45
Val Ser Gln Gln Val Thr Val Gln Ser Ser Pro Asn Phe Thr Gln His
        50 55 60
Val Arg Glu Gln Ser Leu Val Thr Asp Gln Leu Ser Arg Arg Leu Ile
65 70 75 80
Arg Thr Tyr Gln Leu Tyr Ser Arg Thr Ser Gly Lys His Val Gln Val
                                85 90 95
Leu Ala Asn Lys Arg Ile Asn Ala Met Ala Glu Asp Gly Asp Pro Phe
                        100 105 110
Ala Lys Leu Ile Val Glu Thr Asp Thr Phe Gly Ser Arg Val Arg Val
                115 120 125
Arg Gly Ala Glu Thr Gly Leu Tyr Ile Cys Met Asn Lys Lys Gly Lys
        130 135 140
Leu Ile Ala Lys Ser Asn Gly Lys Gly Lys Asp Cys Val Phe Thr Glu
145 150 155 160
Ile Val Leu Glu Asn Asn Tyr Thr Ala Leu Gln Asn Ala Lys Tyr Glu
                                165 170 175
Gly Trp Tyr Met Ala Phe Thr Arg Lys Gly Arg Pro Arg Lys Gly Ser
                        180 185 190
Lys Thr Arg Gln His Gln Arg Glu Val His Phe Met Lys Arg Leu Pro
                195 200 205
Arg Gly His His Thr Thr Glu Gln Ser Leu Arg Phe Glu Phe Leu Asn
        210 215 220
Tyr Pro Pro Phe Thr Arg Ser Leu Arg Gly Ser Gln Arg Thr Trp Ala
225 230 235 240
Pro Glu Pro Arg
<210> SEQ ID NO 149
<211> LENGTH: 140
<212> TYPE: PRT
<213> ORGANISM: Homo sapiens
<400> SEQUENCE: 149
Met Ala Glu Asp Gly Asp Pro Phe Ala Lys Leu Ile Val Glu Thr Asp
1 5 10 15
Thr Phe Gly Ser Arg Val Arg Val Arg Gly Ala Glu Thr Gly Leu Tyr
                        20 25 30
Ile Cys Met Asn Lys Lys Gly Lys Leu Ile Ala Lys Ser Asn Gly Lys
                35 40 45
Gly Lys Asp Cys Val Phe Thr Glu Ile Val Leu Glu Asn Asn Tyr Thr
        50 55 60
Ala Leu Gln Asn Ala Lys Tyr Glu Gly Trp Tyr Met Ala Phe Thr Arg
65 70 75 80
Lys Gly Arg Pro Arg Lys Gly Ser Lys Thr Arg Gln His Gln Arg Glu
                                85 90 95
Val His Phe Met Lys Arg Leu Pro Arg Gly His His Thr Thr Glu Gln
                        100 105 110
Ser Leu Arg Phe Glu Phe Leu Asn Tyr Pro Pro Phe Thr Arg Ser Leu
                115 120 125
Arg Gly Ser Gln Arg Thr Trp Ala Pro Glu Pro Arg
        130 135 140
<210> SEQ ID NO 150
<211> LENGTH: 208
<212> TYPE: PRT
<213> ORGANISM: Homo sapiens
<400> SEQUENCE: 150
Met Ala Pro Leu Gly Glu Val Gly Asn Tyr Phe Gly Val Gln Asp Ala
1 5 10 15
Val Pro Phe Gly Asn Val Pro Val Leu Pro Val Asp Ser Pro Val Leu
                        20 25 30
Leu Ser Asp His Leu Gly Gln Ser Glu Ala Gly Gly Leu Pro Arg Gly
                35 40 45
Pro Ala Val Thr Asp Leu Asp His Leu Lys Gly Ile Leu Arg Arg Arg
        50 55 60
Gln Leu Tyr Cys Arg Thr Gly Phe His Leu Glu Ile Phe Pro Asn Gly
65 70 75 80
Thr Ile Gln Gly Thr Arg Lys Asp His Ser Arg Phe Gly Ile Leu Glu
                                85 90 95
Phe Ile Ser Ile Ala Val Gly Leu Val Ser Ile Arg Gly Val Asp Ser
                        100 105 110
Gly Leu Tyr Leu Gly Met Asn Glu Lys Gly Glu Leu Tyr Gly Ser Glu
                115 120 125
Lys Leu Thr Gln Glu Cys Val Phe Arg Glu Gln Phe Glu Glu Asn Trp
        130 135 140
Tyr Asn Thr Tyr Ser Ser Asn Leu Tyr Lys His Val Asp Thr Gly Arg
145 150 155 160
Arg Tyr Tyr Val Ala Leu Asn Lys Asp Gly Thr Pro Arg Glu Gly Thr
                                165 170 175
Arg Thr Lys Arg His Gln Lys Phe Thr His Phe Leu Pro Arg Pro Val
                        180 185 190
Asp Pro Asp Lys Val Pro Glu Leu Tyr Lys Asp Ile Leu Ser Gln Ser
                195 200 205
<210> SEQ ID NO 151
<211> LENGTH: 208
<212> TYPE: PRT
<213> ORGANISM: Homo sapiens
<400> SEQUENCE: 151
Met Trp Lys Trp Ile Leu Thr His Cys Ala Ser Ala Phe Pro His Leu
1 5 10 15
Pro Gly Cys Cys Cys Cys Cys Phe Leu Leu Leu Phe Leu Val Ser Ser
                        20 25 30
Val Pro Val Thr Cys Gln Ala Leu Gly Gln Asp Met Val Ser Pro Glu
                35 40 45
Ala Thr Asn Ser Ser Ser Ser Ser Phe Ser Ser Pro Ser Ser Ala Gly
        50 55 60
Arg His Val Arg Ser Tyr Asn His Leu Gln Gly Asp Val Arg Trp Arg
65 70 75 80
Lys Leu Phe Ser Phe Thr Lys Tyr Phe Leu Lys Ile Glu Lys Asn Gly
                                85 90 95
Lys Val Ser Gly Thr Lys Lys Glu Asn Cys Pro Tyr Ser Ile Leu Glu
                        100 105 110
Ile Thr Ser Val Glu Ile Gly Val Val Ala Val Lys Ala Ile Asn Ser
                115 120 125
Asn Tyr Tyr Leu Ala Met Asn Lys Lys Gly Lys Leu Tyr Gly Ser Lys
        130 135 140
Glu Phe Asn Asn Asp Cys Lys Leu Lys Glu Arg Ile Glu Glu Asn Gly
145 150 155 160
Tyr Asn Thr Tyr Ala Ser Phe Asn Trp Gln His Asn Gly Arg Gln Met
                                165 170 175
Tyr Val Ala Leu Asn Gly Lys Gly Ala Pro Arg Arg Gly Gln Lys Thr
                        180 185 190
Arg Arg Lys Asn Thr Ser Ala His Phe Leu Pro Met Val Val His Ser
                195 200 205
<210> SEQ ID NO 152
<211> LENGTH: 225
<212> TYPE: PRT
<213> ORGANISM: Homo sapiens
<400> SEQUENCE: 152
Met Ala Ala Leu Ala Ser Ser Leu Ile Arg Gln Lys Arg Glu Val Arg
1 5 10 15
Glu Pro Gly Gly Ser Arg Pro Val Ser Ala Gln Arg Arg Val Cys Pro
                        20 25 30
Arg Gly Thr Lys Ser Leu Cys Gln Lys Gln Leu Leu Ile Leu Leu Ser
                35 40 45
Lys Val Arg Leu Cys Gly Gly Arg Pro Ala Arg Pro Asp Arg Gly Pro
        50 55 60
Glu Pro Gln Leu Lys Gly Ile Val Thr Lys Leu Phe Cys Arg Gln Gly
65 70 75 80
Phe Tyr Leu Gln Ala Asn Pro Asp Gly Ser Ile Gln Gly Thr Pro Glu
                                85 90 95
Asp Thr Ser Ser Phe Thr His Phe Asn Leu Ile Pro Val Gly Leu Arg
                        100 105 110
Val Val Thr Ile Gln Ser Ala Lys Leu Gly His Tyr Met Ala Met Asn
                115 120 125
Ala Glu Gly Leu Leu Tyr Ser Ser Pro His Phe Thr Ala Glu Cys Arg
        130 135 140
Phe Lys Glu Cys Val Phe Glu Asn Tyr Tyr Val Leu Tyr Ala Ser Ala
145 150 155 160
Leu Tyr Arg Gln Arg Arg Ser Gly Arg Ala Trp Tyr Leu Gly Leu Asp
                                165 170 175
Lys Glu Gly Gln Val Met Lys Gly Asn Arg Val Lys Lys Thr Lys Ala
                        180 185 190
Ala Ala His Phe Leu Pro Lys Leu Leu Glu Val Ala Met Tyr Gln Glu
                195 200 205
Pro Ser Leu His Ser Val Pro Glu Ala Ser Pro Ser Ser Pro Pro Ala
        210 215 220
Pro
225
<210> SEQ ID NO 153
<211> LENGTH: 243
<212> TYPE: PRT
<213> ORGANISM: Homo sapiens
<400> SEQUENCE: 153
Met Ala Ala Ala Ile Ala Ser Ser Leu Ile Arg Gln Lys Arg Gln Ala
1 5 10 15
Arg Glu Ser Asn Ser Asp Arg Val Ser Ala Ser Lys Arg Arg Ser Ser
                        20 25 30
Pro Ser Lys Asp Gly Arg Ser Leu Cys Glu Arg His Val Leu Gly Val
                35 40 45
Phe Ser Lys Val Arg Phe Cys Ser Gly Arg Lys Arg Pro Val Arg Arg
        50 55 60
Arg Pro Glu Pro Gln Leu Lys Gly Ile Val Thr Arg Leu Phe Ser Gln
65 70 75 80
Gln Gly Tyr Phe Leu Gln Met His Pro Asp Gly Thr Ile Asp Gly Thr
                                85 90 95
Lys Asp Glu Asn Ser Asp Tyr Thr Leu Phe Asn Leu Ile Pro Val Gly
                        100 105 110
Leu Arg Val Val Ala Ile Gln Gly Val Lys Ala Ser Leu Tyr Val Ala
                115 120 125
Met Asn Gly Glu Gly Tyr Leu Tyr Ser Ser Asp Val Phe Thr Pro Glu
        130 135 140
Cys Lys Phe Lys Glu Ser Val Phe Glu Asn Tyr Tyr Val Ile Tyr Ser
145 150 155 160
Ser Thr Leu Tyr Arg Gln Gln Glu Ser Gly Arg Ala Trp Phe Leu Gly
                                165 170 175
Leu Asn Lys Glu Gly Gln Ile Met Lys Gly Asn Arg Val Lys Lys Thr
                        180 185 190
Lys Pro Ser Ser His Phe Val Pro Lys Pro Ile Glu Val Cys Met Tyr
                195 200 205
Arg Glu Pro Ser Leu His Glu Ile Gly Glu Lys Gln Gly Arg Ser Arg
        210 215 220
Lys Ser Ser Gly Thr Pro Thr Met Asn Gly Gly Lys Val Val Asn Gln
225 230 235 240
Asp Ser Thr
<210> SEQ ID NO 154
<211> LENGTH: 181
<212> TYPE: PRT
<213> ORGANISM: Homo sapiens
<400> SEQUENCE: 154
Met Glu Ser Lys Glu Pro Gln Leu Lys Gly Ile Val Thr Arg Leu Phe
1 5 10 15
Ser Gln Gln Gly Tyr Phe Leu Gln Met His Pro Asp Gly Thr Ile Asp
                        20 25 30
Gly Thr Lys Asp Glu Asn Ser Asp Tyr Thr Leu Phe Asn Leu Ile Pro
                35 40 45
Val Gly Leu Arg Val Val Ala Ile Gln Gly Val Lys Ala Ser Leu Tyr
        50 55 60
Val Ala Met Asn Gly Glu Gly Tyr Leu Tyr Ser Ser Asp Val Phe Thr
65 70 75 80
Pro Glu Cys Lys Phe Lys Glu Ser Val Phe Glu Asn Tyr Tyr Val Ile
                                85 90 95
Tyr Ser Ser Thr Leu Tyr Arg Gln Gln Glu Ser Gly Arg Ala Trp Phe
                        100 105 110
Leu Gly Leu Asn Lys Glu Gly Gln Ile Met Lys Gly Asn Arg Val Lys
                115 120 125
Lys Thr Lys Pro Ser Ser His Phe Val Pro Lys Pro Ile Glu Val Cys
        130 135 140
Met Tyr Arg Glu Pro Ser Leu His Glu Ile Gly Glu Lys Gln Gly Arg
145 150 155 160
Ser Arg Lys Ser Ser Gly Thr Pro Thr Met Asn Gly Gly Lys Val Val
                                165 170 175
Asn Gln Asp Ser Thr
                        180
<210> SEQ ID NO 155
<211> LENGTH: 245
<212> TYPE: PRT
<213> ORGANISM: Homo sapiens
<400> SEQUENCE: 155
Met Ala Ala Ala Ile Ala Ser Ser Leu Ile Arg Gln Lys Arg Gln Ala
1 5 10 15
Arg Glu Arg Glu Lys Ser Asn Ala Cys Lys Cys Val Ser Ser Pro Ser
                        20 25 30
Lys Gly Lys Thr Ser Cys Asp Lys Asn Lys Leu Asn Val Phe Ser Arg
                35 40 45
Val Lys Leu Phe Gly Ser Lys Lys Arg Arg Arg Arg Arg Pro Glu Pro
        50 55 60
Gln Leu Lys Gly Ile Val Thr Lys Leu Tyr Ser Arg Gln Gly Tyr His
65 70 75 80
Leu Gln Leu Gln Ala Asp Gly Thr Ile Asp Gly Thr Lys Asp Glu Asp
                                85 90 95
Ser Thr Tyr Thr Leu Phe Asn Leu Ile Pro Val Gly Leu Arg Val Val
                        100 105 110
Ala Ile Gln Gly Val Gln Thr Lys Leu Tyr Leu Ala Met Asn Ser Glu
                115 120 125
Gly Tyr Leu Tyr Thr Ser Glu Leu Phe Thr Pro Glu Cys Lys Phe Lys
        130 135 140
Glu Ser Val Phe Glu Asn Tyr Tyr Val Thr Tyr Ser Ser Met Ile Tyr
145 150 155 160
Arg Gln Gln Gln Ser Gly Arg Gly Trp Tyr Leu Gly Leu Asn Lys Glu
                                165 170 175
Gly Glu Ile Met Lys Gly Asn His Val Lys Lys Asn Lys Pro Ala Ala
                        180 185 190
His Phe Leu Pro Lys Pro Leu Lys Val Ala Met Tyr Lys Glu Pro Ser
                195 200 205
Leu His Asp Leu Thr Glu Phe Ser Arg Ser Gly Ser Gly Thr Pro Thr
        210 215 220
Lys Ser Arg Ser Val Ser Gly Val Leu Asn Gly Gly Lys Ser Met Ser
225 230 235 240
His Asn Glu Ser Thr
                                245
<210> SEQ ID NO 156
<211> LENGTH: 255
<212> TYPE: PRT
<213> ORGANISM: Homo sapiens
<400> SEQUENCE: 156
Met Ser Gly Lys Val Thr Lys Pro Lys Glu Glu Lys Asp Ala Ser Lys
1 5 10 15
Val Leu Asp Asp Ala Pro Pro Gly Thr Gln Glu Tyr Ile Met Leu Arg
                        20 25 30
Gln Asp Ser Ile Gln Ser Ala Glu Leu Lys Lys Lys Glu Ser Pro Phe
                35 40 45
Arg Ala Lys Cys His Glu Ile Phe Cys Cys Pro Leu Lys Gln Val His
        50 55 60
His Lys Glu Asn Thr Glu Pro Glu Glu Pro Gln Leu Lys Gly Ile Val
65 70 75 80
Thr Lys Leu Tyr Ser Arg Gln Gly Tyr His Leu Gln Leu Gln Ala Asp
                                85 90 95
Gly Thr Ile Asp Gly Thr Lys Asp Glu Asp Ser Thr Tyr Thr Leu Phe
                        100 105 110
Asn Leu Ile Pro Val Gly Leu Arg Val Val Ala Ile Gln Gly Val Gln
                115 120 125
Thr Lys Leu Tyr Leu Ala Met Asn Ser Glu Gly Tyr Leu Tyr Thr Ser
        130 135 140
Glu Leu Phe Thr Pro Glu Cys Lys Phe Lys Glu Ser Val Phe Glu Asn
145 150 155 160
Tyr Tyr Val Thr Tyr Ser Ser Met Ile Tyr Arg Gln Gln Gln Ser Gly
                                165 170 175
Arg Gly Trp Tyr Leu Gly Leu Asn Lys Glu Gly Glu Ile Met Lys Gly
                        180 185 190
Asn His Val Lys Lys Asn Lys Pro Ala Ala His Phe Leu Pro Lys Pro
                195 200 205
Leu Lys Val Ala Met Tyr Lys Glu Pro Ser Leu His Asp Leu Thr Glu
        210 215 220
Phe Ser Arg Ser Gly Ser Gly Thr Pro Thr Lys Ser Arg Ser Val Ser
225 230 235 240
Gly Val Leu Asn Gly Gly Lys Ser Met Ser His Asn Glu Ser Thr
                                245 250 255
<210> SEQ ID NO 157
<211> LENGTH: 226
<212> TYPE: PRT
<213> ORGANISM: Homo sapiens
<400> SEQUENCE: 157
Met Leu Arg Gln Asp Ser Ile Gln Ser Ala Glu Leu Lys Lys Lys Glu
1 5 10 15
Ser Pro Phe Arg Ala Lys Cys His Glu Ile Phe Cys Cys Pro Leu Lys
                        20 25 30
Gln Val His His Lys Glu Asn Thr Glu Pro Glu Glu Pro Gln Leu Lys
                35 40 45
Gly Ile Val Thr Lys Leu Tyr Ser Arg Gln Gly Tyr His Leu Gln Leu
        50 55 60
Gln Ala Asp Gly Thr Ile Asp Gly Thr Lys Asp Glu Asp Ser Thr Tyr
65 70 75 80
Thr Leu Phe Asn Leu Ile Pro Val Gly Leu Arg Val Val Ala Ile Gln
                                85 90 95
Gly Val Gln Thr Lys Leu Tyr Leu Ala Met Asn Ser Glu Gly Tyr Leu
                        100 105 110
Tyr Thr Ser Glu Leu Phe Thr Pro Glu Cys Lys Phe Lys Glu Ser Val
                115 120 125
Phe Glu Asn Tyr Tyr Val Thr Tyr Ser Ser Met Ile Tyr Arg Gln Gln
        130 135 140
Gln Ser Gly Arg Gly Trp Tyr Leu Gly Leu Asn Lys Glu Gly Glu Ile
145 150 155 160
Met Lys Gly Asn His Val Lys Lys Asn Lys Pro Ala Ala His Phe Leu
                                165 170 175
Pro Lys Pro Leu Lys Val Ala Met Tyr Lys Glu Pro Ser Leu His Asp
                        180 185 190
Leu Thr Glu Phe Ser Arg Ser Gly Ser Gly Thr Pro Thr Lys Ser Arg
                195 200 205
Ser Val Ser Gly Val Leu Asn Gly Gly Lys Ser Met Ser His Asn Glu
        210 215 220
Ser Thr
225
<210> SEQ ID NO 158
<211> LENGTH: 199
<212> TYPE: PRT
<213> ORGANISM: Homo sapiens
<400> SEQUENCE: 158
Met Ser Gly Lys Val Thr Lys Pro Lys Glu Glu Lys Asp Ala Ser Lys
1 5 10 15
Glu Pro Gln Leu Lys Gly Ile Val Thr Lys Leu Tyr Ser Arg Gln Gly
                        20 25 30
Tyr His Leu Gln Leu Gln Ala Asp Gly Thr Ile Asp Gly Thr Lys Asp
                35 40 45
Glu Asp Ser Thr Tyr Thr Leu Phe Asn Leu Ile Pro Val Gly Leu Arg
        50 55 60
Val Val Ala Ile Gln Gly Val Gln Thr Lys Leu Tyr Leu Ala Met Asn
65 70 75 80
Ser Glu Gly Tyr Leu Tyr Thr Ser Glu Leu Phe Thr Pro Glu Cys Lys
                                85 90 95
Phe Lys Glu Ser Val Phe Glu Asn Tyr Tyr Val Thr Tyr Ser Ser Met
                        100 105 110
Ile Tyr Arg Gln Gln Gln Ser Gly Arg Gly Trp Tyr Leu Gly Leu Asn
                115 120 125
Lys Glu Gly Glu Ile Met Lys Gly Asn His Val Lys Lys Asn Lys Pro
        130 135 140
Ala Ala His Phe Leu Pro Lys Pro Leu Lys Val Ala Met Tyr Lys Glu
145 150 155 160
Pro Ser Leu His Asp Leu Thr Glu Phe Ser Arg Ser Gly Ser Gly Thr
                                165 170 175
Pro Thr Lys Ser Arg Ser Val Ser Gly Val Leu Asn Gly Gly Lys Ser
                        180 185 190
Met Ser His Asn Glu Ser Thr
                195
<210> SEQ ID NO 159
<211> LENGTH: 226
<212> TYPE: PRT
<213> ORGANISM: Homo sapiens
<400> SEQUENCE: 159
Met Leu Arg Gln Asp Ser Ile Gln Ser Ala Glu Leu Lys Lys Lys Glu
1 5 10 15
Ser Pro Phe Arg Ala Lys Cys His Glu Ile Phe Cys Cys Pro Leu Lys
                        20 25 30
Gln Val His His Lys Glu Asn Thr Glu Pro Glu Glu Pro Gln Leu Lys
                35 40 45
Gly Ile Val Thr Lys Leu Tyr Ser Arg Gln Gly Tyr His Leu Gln Leu
        50 55 60
Gln Ala Asp Gly Thr Ile Asp Gly Thr Lys Asp Glu Asp Ser Thr Tyr
65 70 75 80
Thr Leu Phe Asn Leu Ile Pro Val Gly Leu Arg Val Val Ala Ile Gln
                                85 90 95
Gly Val Gln Thr Lys Leu Tyr Leu Ala Met Asn Ser Glu Gly Tyr Leu
                        100 105 110
Tyr Thr Ser Glu Leu Phe Thr Pro Glu Cys Lys Phe Lys Glu Ser Val
                115 120 125
Phe Glu Asn Tyr Tyr Val Thr Tyr Ser Ser Met Ile Tyr Arg Gln Gln
        130 135 140
Gln Ser Gly Arg Gly Trp Tyr Leu Gly Leu Asn Lys Glu Gly Glu Ile
145 150 155 160
Met Lys Gly Asn His Val Lys Lys Asn Lys Pro Ala Ala His Phe Leu
                                165 170 175
Pro Lys Pro Leu Lys Val Ala Met Tyr Lys Glu Pro Ser Leu His Asp
                        180 185 190
Leu Thr Glu Phe Ser Arg Ser Gly Ser Gly Thr Pro Thr Lys Ser Arg
                195 200 205
Ser Val Ser Gly Val Leu Asn Gly Gly Lys Ser Met Ser His Asn Glu
        210 215 220
Ser Thr
225
<210> SEQ ID NO 160
<211> LENGTH: 192
<212> TYPE: PRT
<213> ORGANISM: Homo sapiens
<400> SEQUENCE: 160
Met Ala Leu Leu Arg Lys Ser Tyr Ser Glu Pro Gln Leu Lys Gly Ile
1 5 10 15
Val Thr Lys Leu Tyr Ser Arg Gln Gly Tyr His Leu Gln Leu Gln Ala
                        20 25 30
Asp Gly Thr Ile Asp Gly Thr Lys Asp Glu Asp Ser Thr Tyr Thr Leu
                35 40 45
Phe Asn Leu Ile Pro Val Gly Leu Arg Val Val Ala Ile Gln Gly Val
        50 55 60
Gln Thr Lys Leu Tyr Leu Ala Met Asn Ser Glu Gly Tyr Leu Tyr Thr
65 70 75 80
Ser Glu Leu Phe Thr Pro Glu Cys Lys Phe Lys Glu Ser Val Phe Glu
                                85 90 95
Asn Tyr Tyr Val Thr Tyr Ser Ser Met Ile Tyr Arg Gln Gln Gln Ser
                        100 105 110
Gly Arg Gly Trp Tyr Leu Gly Leu Asn Lys Glu Gly Glu Ile Met Lys
                115 120 125
Gly Asn His Val Lys Lys Asn Lys Pro Ala Ala His Phe Leu Pro Lys
        130 135 140
Pro Leu Lys Val Ala Met Tyr Lys Glu Pro Ser Leu His Asp Leu Thr
145 150 155 160
Glu Phe Ser Arg Ser Gly Ser Gly Thr Pro Thr Lys Ser Arg Ser Val
                                165 170 175
Ser Gly Val Leu Asn Gly Gly Lys Ser Met Ser His Asn Glu Ser Thr
                        180 185 190
<210> SEQ ID NO 161
<211> LENGTH: 247
<212> TYPE: PRT
<213> ORGANISM: Homo sapiens
<400> SEQUENCE: 161
Met Ala Ala Ala Ile Ala Ser Gly Leu Ile Arg Gln Lys Arg Gln Ala
1 5 10 15
Arg Glu Gln His Trp Asp Arg Pro Ser Ala Ser Arg Arg Arg Ser Ser
                        20 25 30
Pro Ser Lys Asn Arg Gly Leu Cys Asn Gly Asn Leu Val Asp Ile Phe
                35 40 45
Ser Lys Val Arg Ile Phe Gly Leu Lys Lys Arg Arg Leu Arg Arg Gln
        50 55 60
Asp Pro Gln Leu Lys Gly Ile Val Thr Arg Leu Tyr Cys Arg Gln Gly
65 70 75 80
Tyr Tyr Leu Gln Met His Pro Asp Gly Ala Leu Asp Gly Thr Lys Asp
                                85 90 95
Asp Ser Thr Asn Ser Thr Leu Phe Asn Leu Ile Pro Val Gly Leu Arg
                        100 105 110
Val Val Ala Ile Gln Gly Val Lys Thr Gly Leu Tyr Ile Ala Met Asn
                115 120 125
Gly Glu Gly Tyr Leu Tyr Pro Ser Glu Leu Phe Thr Pro Glu Cys Lys
        130 135 140
Phe Lys Glu Ser Val Phe Glu Asn Tyr Tyr Val Ile Tyr Ser Ser Met
145 150 155 160
Leu Tyr Arg Gln Gln Glu Ser Gly Arg Ala Trp Phe Leu Gly Leu Asn
                                165 170 175
Lys Glu Gly Gln Ala Met Lys Gly Asn Arg Val Lys Lys Thr Lys Pro
                        180 185 190
Ala Ala His Phe Leu Pro Lys Pro Leu Glu Val Ala Met Tyr Arg Glu
                195 200 205
Pro Ser Leu His Asp Val Gly Glu Thr Val Pro Lys Pro Gly Val Thr
        210 215 220
Pro Ser Lys Ser Thr Ser Ala Ser Ala Ile Met Asn Gly Gly Lys Pro
225 230 235 240
Val Asn Lys Ser Lys Thr Thr
                                245
<210> SEQ ID NO 162
<211> LENGTH: 252
<212> TYPE: PRT
<213> ORGANISM: Homo sapiens
<400> SEQUENCE: 162
Met Val Lys Pro Val Pro Leu Phe Arg Arg Thr Asp Phe Lys Leu Leu
1 5 10 15
Leu Cys Asn His Lys Asp Leu Phe Phe Leu Arg Val Ser Lys Leu Leu
                        20 25 30
Asp Cys Phe Ser Pro Lys Ser Met Trp Phe Leu Trp Asn Ile Phe Ser
                35 40 45
Lys Gly Thr His Met Leu Gln Cys Leu Cys Gly Lys Ser Leu Lys Lys
        50 55 60
Asn Lys Asn Pro Thr Asp Pro Gln Leu Lys Gly Ile Val Thr Arg Leu
65 70 75 80
Tyr Cys Arg Gln Gly Tyr Tyr Leu Gln Met His Pro Asp Gly Ala Leu
                                85 90 95
Asp Gly Thr Lys Asp Asp Ser Thr Asn Ser Thr Leu Phe Asn Leu Ile
                        100 105 110
Pro Val Gly Leu Arg Val Val Ala Ile Gln Gly Val Lys Thr Gly Leu
                115 120 125
Tyr Ile Ala Met Asn Gly Glu Gly Tyr Leu Tyr Pro Ser Glu Leu Phe
        130 135 140
Thr Pro Glu Cys Lys Phe Lys Glu Ser Val Phe Glu Asn Tyr Tyr Val
145 150 155 160
Ile Tyr Ser Ser Met Leu Tyr Arg Gln Gln Glu Ser Gly Arg Ala Trp
                                165 170 175
Phe Leu Gly Leu Asn Lys Glu Gly Gln Ala Met Lys Gly Asn Arg Val
                        180 185 190
Lys Lys Thr Lys Pro Ala Ala His Phe Leu Pro Lys Pro Leu Glu Val
                195 200 205
Ala Met Tyr Arg Glu Pro Ser Leu His Asp Val Gly Glu Thr Val Pro
        210 215 220
Lys Pro Gly Val Thr Pro Ser Lys Ser Thr Ser Ala Ser Ala Ile Met
225 230 235 240
Asn Gly Gly Lys Pro Val Asn Lys Ser Lys Thr Thr
                                245 250
<210> SEQ ID NO 163
<211> LENGTH: 207
<212> TYPE: PRT
<213> ORGANISM: Homo sapiens
<400> SEQUENCE: 163
Met Ala Glu Val Gly Gly Val Phe Ala Ser Leu Asp Trp Asp Leu His
1 5 10 15
Gly Phe Ser Ser Ser Leu Gly Asn Val Pro Leu Ala Asp Ser Pro Gly
                        20 25 30
Phe Leu Asn Glu Arg Leu Gly Gln Ile Glu Gly Lys Leu Gln Arg Gly
                35 40 45
Ser Pro Thr Asp Phe Ala His Leu Lys Gly Ile Leu Arg Arg Arg Gln
        50 55 60
Leu Tyr Cys Arg Thr Gly Phe His Leu Glu Ile Phe Pro Asn Gly Thr
65 70 75 80
Val His Gly Thr Arg His Asp His Ser Arg Phe Gly Ile Leu Glu Phe
                                85 90 95
Ile Ser Leu Ala Val Gly Leu Ile Ser Ile Arg Gly Val Asp Ser Gly
                        100 105 110
Leu Tyr Leu Gly Met Asn Glu Arg Gly Glu Leu Tyr Gly Ser Lys Lys
                115 120 125
Leu Thr Arg Glu Cys Val Phe Arg Glu Gln Phe Glu Glu Asn Trp Tyr
        130 135 140
Asn Thr Tyr Ala Ser Thr Leu Tyr Lys His Ser Asp Ser Glu Arg Gln
145 150 155 160
Tyr Tyr Val Ala Leu Asn Lys Asp Gly Ser Pro Arg Glu Gly Tyr Arg
                                165 170 175
Thr Lys Arg His Gln Lys Phe Thr His Phe Leu Pro Arg Pro Val Asp
                        180 185 190
Pro Ser Lys Leu Pro Ser Met Ser Arg Asp Leu Phe His Tyr Arg
                195 200 205
<210> SEQ ID NO 164
<211> LENGTH: 216
<212> TYPE: PRT
<213> ORGANISM: Homo sapiens
<400> SEQUENCE: 164
Met Gly Ala Ala Arg Leu Leu Pro Asn Leu Thr Leu Cys Leu Gln Leu
1 5 10 15
Leu Ile Leu Cys Cys Gln Thr Gln Gly Glu Asn His Pro Ser Pro Asn
                        20 25 30
Phe Asn Gln Tyr Val Arg Asp Gln Gly Ala Met Thr Asp Gln Leu Ser
                35 40 45
Arg Arg Gln Ile Arg Glu Tyr Gln Leu Tyr Ser Arg Thr Ser Gly Lys
        50 55 60
His Val Gln Val Thr Gly Arg Arg Ile Ser Ala Thr Ala Glu Asp Gly
65 70 75 80
Asn Lys Phe Ala Lys Leu Ile Val Glu Thr Asp Thr Phe Gly Ser Arg
                                85 90 95
Val Arg Ile Lys Gly Ala Glu Ser Glu Lys Tyr Ile Cys Met Asn Lys
                        100 105 110
Arg Gly Lys Leu Ile Gly Lys Pro Ser Gly Lys Ser Lys Asp Cys Val
                115 120 125
Phe Thr Glu Ile Val Leu Glu Asn Asn Tyr Thr Ala Phe Gln Asn Ala
        130 135 140
Arg His Glu Gly Trp Phe Met Ala Phe Thr Arg Gln Gly Arg Pro Arg
145 150 155 160
Gln Ala Ser Arg Ser Arg Gln Asn Gln Arg Glu Ala His Phe Ile Lys
                                165 170 175
Arg Leu Tyr Gln Gly Gln Leu Pro Phe Pro Asn His Ala Glu Lys Gln
                        180 185 190
Lys Gln Phe Glu Phe Val Gly Ser Ala Pro Thr Arg Arg Thr Lys Arg
                195 200 205
Thr Arg Arg Pro Gln Pro Leu Thr
        210 215
<210> SEQ ID NO 165
<211> LENGTH: 207
<212> TYPE: PRT
<213> ORGANISM: Homo sapiens
<400> SEQUENCE: 165
Met Tyr Ser Ala Pro Ser Ala Cys Thr Cys Leu Cys Leu His Phe Leu
1 5 10 15
Leu Leu Cys Phe Gln Val Gln Val Leu Val Ala Glu Glu Asn Val Asp
                        20 25 30
Phe Arg Ile His Val Glu Asn Gln Thr Arg Ala Arg Asp Asp Val Ser
                35 40 45
Arg Lys Gln Leu Arg Leu Tyr Gln Leu Tyr Ser Arg Thr Ser Gly Lys
        50 55 60
His Ile Gln Val Leu Gly Arg Arg Ile Ser Ala Arg Gly Glu Asp Gly
65 70 75 80
Asp Lys Tyr Ala Gln Leu Leu Val Glu Thr Asp Thr Phe Gly Ser Gln
                                85 90 95
Val Arg Ile Lys Gly Lys Glu Thr Glu Phe Tyr Leu Cys Met Asn Arg
                        100 105 110
Lys Gly Lys Leu Val Gly Lys Pro Asp Gly Thr Ser Lys Glu Cys Val
                115 120 125
Phe Ile Glu Lys Val Leu Glu Asn Asn Tyr Thr Ala Leu Met Ser Ala
        130 135 140
Lys Tyr Ser Gly Trp Tyr Val Gly Phe Thr Lys Lys Gly Arg Pro Arg
145 150 155 160
Lys Gly Pro Lys Thr Arg Glu Asn Gln Gln Asp Val His Phe Met Lys
                                165 170 175
Arg Tyr Pro Lys Gly Gln Pro Glu Leu Gln Lys Pro Phe Lys Tyr Thr
                        180 185 190
Thr Val Thr Lys Arg Ser Arg Arg Ile Arg Pro Thr His Pro Ala
                195 200 205
<210> SEQ ID NO 166
<211> LENGTH: 216
<212> TYPE: PRT
<213> ORGANISM: Homo sapiens
<400> SEQUENCE: 166
Met Arg Ser Gly Cys Val Val Val His Val Trp Ile Leu Ala Gly Leu
1 5 10 15
Trp Leu Ala Val Ala Gly Arg Pro Leu Ala Phe Ser Asp Ala Gly Pro
                        20 25 30
His Val His Tyr Gly Trp Gly Asp Pro Ile Arg Leu Arg His Leu Tyr
                35 40 45
Thr Ser Gly Pro His Gly Leu Ser Ser Cys Phe Leu Arg Ile Arg Ala
        50 55 60
Asp Gly Val Val Asp Cys Ala Arg Gly Gln Ser Ala His Ser Leu Leu
65 70 75 80
Glu Ile Lys Ala Val Ala Leu Arg Thr Val Ala Ile Lys Gly Val His
                                85 90 95
Ser Val Arg Tyr Leu Cys Met Gly Ala Asp Gly Lys Met Gln Gly Leu
                        100 105 110
Leu Gln Tyr Ser Glu Glu Asp Cys Ala Phe Glu Glu Glu Ile Arg Pro
                115 120 125
Asp Gly Tyr Asn Val Tyr Arg Ser Glu Lys His Arg Leu Pro Val Ser
        130 135 140
Leu Ser Ser Ala Lys Gln Arg Gln Leu Tyr Lys Asn Arg Gly Phe Leu
145 150 155 160
Pro Leu Ser His Phe Leu Pro Met Leu Pro Met Val Pro Glu Glu Pro
                                165 170 175
Glu Asp Leu Arg Gly His Leu Glu Ser Asp Met Phe Ser Ser Pro Leu
                        180 185 190
Glu Thr Asp Ser Met Asp Pro Phe Gly Leu Val Thr Gly Leu Glu Ala
                195 200 205
Val Arg Ser Pro Ser Phe Glu Lys
        210 215
<210> SEQ ID NO 167
<211> LENGTH: 211
<212> TYPE: PRT
<213> ORGANISM: Homo sapiens
<400> SEQUENCE: 167
Met Ala Pro Leu Ala Glu Val Gly Gly Phe Leu Gly Gly Leu Glu Gly
1 5 10 15
Leu Gly Gln Gln Val Gly Ser His Phe Leu Leu Pro Pro Ala Gly Glu
                        20 25 30
Arg Pro Pro Leu Leu Gly Glu Arg Arg Ser Ala Ala Glu Arg Ser Ala
                35 40 45
Arg Gly Gly Pro Gly Ala Ala Gln Leu Ala His Leu His Gly Ile Leu
        50 55 60
Arg Arg Arg Gln Leu Tyr Cys Arg Thr Gly Phe His Leu Gln Ile Leu
65 70 75 80
Pro Asp Gly Ser Val Gln Gly Thr Arg Gln Asp His Ser Leu Phe Gly
                                85 90 95
Ile Leu Glu Phe Ile Ser Val Ala Val Gly Leu Val Ser Ile Arg Gly
                        100 105 110
Val Asp Ser Gly Leu Tyr Leu Gly Met Asn Asp Lys Gly Glu Leu Tyr
                115 120 125
Gly Ser Glu Lys Leu Thr Ser Glu Cys Ile Phe Arg Glu Gln Phe Glu
        130 135 140
Glu Asn Trp Tyr Asn Thr Tyr Ser Ser Asn Ile Tyr Lys His Gly Asp
145 150 155 160
Thr Gly Arg Arg Tyr Phe Val Ala Leu Asn Lys Asp Gly Thr Pro Arg
                                165 170 175
Asp Gly Ala Arg Ser Lys Arg His Gln Lys Phe Thr His Phe Leu Pro
                        180 185 190
Arg Pro Val Asp Pro Glu Arg Val Pro Glu Leu Tyr Lys Asp Leu Leu
                195 200 205
Met Tyr Thr
        210
<210> SEQ ID NO 168
<211> LENGTH: 209
<212> TYPE: PRT
<213> ORGANISM: Homo sapiens
<400> SEQUENCE: 168
Met Asp Ser Asp Glu Thr Gly Phe Glu His Ser Gly Leu Trp Val Ser
1 5 10 15
Val Leu Ala Gly Leu Leu Leu Gly Ala Cys Gln Ala His Pro Ile Pro
                        20 25 30
Asp Ser Ser Pro Leu Leu Gln Phe Gly Gly Gln Val Arg Gln Arg Tyr
                35 40 45
Leu Tyr Thr Asp Asp Ala Gln Gln Thr Glu Ala His Leu Glu Ile Arg
        50 55 60
Glu Asp Gly Thr Val Gly Gly Ala Ala Asp Gln Ser Pro Glu Ser Leu
65 70 75 80
Leu Gln Leu Lys Ala Leu Lys Pro Gly Val Ile Gln Ile Leu Gly Val
                                85 90 95
Lys Thr Ser Arg Phe Leu Cys Gln Arg Pro Asp Gly Ala Leu Tyr Gly
                        100 105 110
Ser Leu His Phe Asp Pro Glu Ala Cys Ser Phe Arg Glu Leu Leu Leu
                115 120 125
Glu Asp Gly Tyr Asn Val Tyr Gln Ser Glu Ala His Gly Leu Pro Leu
        130 135 140
His Leu Pro Gly Asn Lys Ser Pro His Arg Asp Pro Ala Pro Arg Gly
145 150 155 160
Pro Ala Arg Phe Leu Pro Leu Pro Gly Leu Pro Pro Ala Leu Pro Glu
                                165 170 175
Pro Pro Gly Ile Leu Ala Pro Gln Pro Pro Asp Val Gly Ser Ser Asp
                        180 185 190
Pro Leu Ser Met Val Gly Pro Ser Gln Gly Arg Ser Pro Ser Tyr Ala
                195 200 205
Ser
<210> SEQ ID NO 169
<211> LENGTH: 170
<212> TYPE: PRT
<213> ORGANISM: Homo sapiens
<400> SEQUENCE: 169
Met Arg Arg Arg Leu Trp Leu Gly Leu Ala Trp Leu Leu Leu Ala Arg
1 5 10 15
Ala Pro Asp Ala Ala Gly Thr Pro Ser Ala Ser Arg Gly Pro Arg Ser
                        20 25 30
Tyr Pro His Leu Glu Gly Asp Val Arg Trp Arg Arg Leu Phe Ser Ser
                35 40 45
Thr His Phe Phe Leu Arg Val Asp Pro Gly Gly Arg Val Gln Gly Thr
        50 55 60
Arg Trp Arg His Gly Gln Asp Ser Ile Leu Glu Ile Arg Ser Val His
65 70 75 80
Val Gly Val Val Val Ile Lys Ala Val Ser Ser Gly Phe Tyr Val Ala
                                85 90 95
Met Asn Arg Arg Gly Arg Leu Tyr Gly Ser Arg Leu Tyr Thr Val Asp
                        100 105 110
Cys Arg Phe Arg Glu Arg Ile Glu Glu Asn Gly His Asn Thr Tyr Ala
                115 120 125
Ser Gln Arg Trp Arg Arg Arg Gly Gln Pro Met Phe Leu Ala Leu Asp
        130 135 140
Arg Arg Gly Gly Pro Arg Pro Gly Gly Arg Thr Arg Arg Tyr His Leu
145 150 155 160
Ser Ala His Phe Leu Pro Val Leu Val Ser
                                165 170
<210> SEQ ID NO 170
<211> LENGTH: 251
<212> TYPE: PRT
<213> ORGANISM: Homo sapiens
<400> SEQUENCE: 170
Met Leu Gly Ala Arg Leu Arg Leu Trp Val Cys Ala Leu Cys Ser Val
1 5 10 15
Cys Ser Met Ser Val Leu Arg Ala Tyr Pro Asn Ala Ser Pro Leu Leu
                        20 25 30
Gly Ser Ser Trp Gly Gly Leu Ile His Leu Tyr Thr Ala Thr Ala Arg
                35 40 45
Asn Ser Tyr His Leu Gln Ile His Lys Asn Gly His Val Asp Gly Ala
        50 55 60
Pro His Gln Thr Ile Tyr Ser Ala Leu Met Ile Arg Ser Glu Asp Ala
65 70 75 80
Gly Phe Val Val Ile Thr Gly Val Met Ser Arg Arg Tyr Leu Cys Met
                                85 90 95
Asp Phe Arg Gly Asn Ile Phe Gly Ser His Tyr Phe Asp Pro Glu Asn
                        100 105 110
Cys Arg Phe Gln His Gln Thr Leu Glu Asn Gly Tyr Asp Val Tyr His
                115 120 125
Ser Pro Gln Tyr His Phe Leu Val Ser Leu Gly Arg Ala Lys Arg Ala
        130 135 140
Phe Leu Pro Gly Met Asn Pro Pro Pro Tyr Ser Gln Phe Leu Ser Arg
145 150 155 160
Arg Asn Glu Ile Pro Leu Ile His Phe Asn Thr Pro Ile Pro Arg Arg
                                165 170 175
His Thr Arg Ser Ala Glu Asp Asp Ser Glu Arg Asp Pro Leu Asn Val
                        180 185 190
Leu Lys Pro Arg Ala Arg Met Thr Pro Ala Pro Ala Ser Cys Ser Gln
                195 200 205
Glu Leu Pro Ser Ala Glu Asp Asn Ser Pro Met Ala Ser Asp Pro Leu
        210 215 220
Gly Val Val Arg Gly Gly Arg Val Asn Thr His Ala Gly Gly Thr Gly
225 230 235 240
Pro Glu Gly Cys Arg Pro Phe Ala Lys Phe Ile
                                245 250
<210> SEQ ID NO 171
<211> LENGTH: 6
<212> TYPE: PRT
<213> ORGANISM: Homo sapiens
<220> FEATURE:
<221> NAME/KEY: MOD_RES
<222> LOCATION: (1)..(1)
<223> OTHER INFORMATION: Lys or Arg
<220> FEATURE:
<221> NAME/KEY: MOD_RES
<222> LOCATION: (2)..(5)
<223> OTHER INFORMATION: Any amino acid
<220> FEATURE:
<221> NAME/KEY: MOD_RES
<222> LOCATION: (6)..(6)
<223> OTHER INFORMATION: Lys or Arg
<400> SEQUENCE: 171
Xaa Xaa Xaa Xaa Xaa Xaa
1 5
<210> SEQ ID NO 172
<211> LENGTH: 8
<212> TYPE: PRT
<213> ORGANISM: Homo sapiens
<220> FEATURE:
<221> NAME/KEY: MOD_RES
<222> LOCATION: (1)..(1)
<223> OTHER INFORMATION: Lys or Arg
<220> FEATURE:
<221> NAME/KEY: MOD_RES
<222> LOCATION: (2)..(7)
<223> OTHER INFORMATION: Any amino acid
<220> FEATURE:
<221> NAME/KEY: MOD_RES
<222> LOCATION: (8)..(8)
<223> OTHER INFORMATION: Lys or Arg
<400> SEQUENCE: 172
Xaa Xaa Xaa Xaa Xaa Xaa Xaa Xaa
1 5
<210> SEQ ID NO 173
<211> LENGTH: 6
<212> TYPE: PRT
<213> ORGANISM: Homo sapiens
<400> SEQUENCE: 173
Leu Val Pro Arg Gly Ser
1 5
<210> SEQ ID NO 174
<211> LENGTH: 800
<212> TYPE: DNA
<213> ORGANISM: Homo sapiens
<400> SEQUENCE: 174
taatacgact cactataggg aaataagaga gaaaagaaga gtaagaagaa atataagagc 60
caccatggcc ggtcccgcga cccaaagccc catgaaactt atggccctgc agttgctgct 120
ttggcactcg gccctctgga cagtccaaga agcgactcct ctcggacctg cctcatcgtt 180
gccgcagtca ttccttttga agtgtctgga gcaggtgcga aagattcagg gcgatggagc 240
cgcactccaa gagaagctct gcgcgacata caaactttgc catcccgagg agctcgtact 300
gctcgggcac agcttgggga ttccctgggc tcctctctcg tcctgtccgt cgcaggcttt 360
gcagttggca gggtgccttt cccagctcca ctccggtttg ttcttgtatc agggactgct 420
gcaagccctt gagggaatct cgccagaatt gggcccgacg ctggacacgt tgcagctcga 480
cgtggcggat ttcgcaacaa ccatctggca gcagatggag gaactgggga tggcacccgc 540
gctgcagccc acgcaggggg caatgccggc ctttgcgtcc gcgtttcagc gcagggcggg 600
tggagtcctc gtagcgagcc accttcaatc atttttggaa gtctcgtacc gggtgctgag 660
acatcttgcg cagccgtgaa gcgctgcctt ctgcggggct tgccttctgg ccatgccctt 720
cttctctccc ttgcacctgt acctcttggt ctttgaataa agcctgagta ggaaggcggc 780
cgctcgagca tgcatctaga 800
<210> SEQ ID NO 175
<211> LENGTH: 758
<212> TYPE: RNA
<213> ORGANISM: Homo sapiens
<400> SEQUENCE: 175
gggaaauaag agagaaaaga agaguaagaa gaaauauaag agccaccaug gccggucccg 60
cgacccaaag ccccaugaaa cuuauggccc ugcaguugcu gcuuuggcac ucggcccucu 120
ggacagucca agaagcgacu ccucucggac cugccucauc guugccgcag ucauuccuuu 180
ugaagugucu ggagcaggug cgaaagauuc agggcgaugg agccgcacuc caagagaagc 240
ucugcgcgac auacaaacuu ugccaucccg aggagcucgu acugcucggg cacagcuugg 300
ggauucccug ggcuccucuc ucguccuguc cgucgcaggc uuugcaguug gcagggugcc 360
uuucccagcu ccacuccggu uuguucuugu aucagggacu gcugcaagcc cuugagggaa 420
ucucgccaga auugggcccg acgcuggaca cguugcagcu cgacguggcg gauuucgcaa 480
caaccaucug gcagcagaug gaggaacugg ggauggcacc cgcgcugcag cccacgcagg 540
gggcaaugcc ggccuuugcg uccgcguuuc agcgcagggc ggguggaguc cucguagcga 600
gccaccuuca aucauuuuug gaagucucgu accgggugcu gagacaucuu gcgcagccgu 660
gaagcgcugc cuucugcggg gcuugccuuc uggccaugcc cuucuucucu cccuugcacc 720
uguaccucuu ggucuuugaa uaaagccuga guaggaag 758
<210> SEQ ID NO 176
<211> LENGTH: 207
<212> TYPE: PRT
<213> ORGANISM: Homo sapiens
<400> SEQUENCE: 176
Met Ala Gly Pro Ala Thr Gln Ser Pro Met Lys Leu Met Ala Leu Gln
1 5 10 15
Leu Leu Leu Trp His Ser Ala Leu Trp Thr Val Gln Glu Ala Thr Pro
                        20 25 30
Leu Gly Pro Ala Ser Ser Leu Pro Gln Ser Phe Leu Leu Lys Cys Leu
                35 40 45
Glu Gln Val Arg Lys Ile Gln Gly Asp Gly Ala Ala Leu Gln Glu Lys
        50 55 60
Leu Val Ser Glu Cys Ala Thr Tyr Lys Leu Cys His Pro Glu Glu Leu
65 70 75 80
Val Leu Leu Gly His Ser Leu Gly Ile Pro Trp Ala Pro Leu Ser Ser
                                85 90 95
Cys Pro Ser Gln Ala Leu Gln Leu Ala Gly Cys Leu Ser Gln Leu His
                        100 105 110
Ser Gly Leu Phe Leu Tyr Gln Gly Leu Leu Gln Ala Leu Glu Gly Ile
                115 120 125
Ser Pro Glu Leu Gly Pro Thr Leu Asp Thr Leu Gln Leu Asp Val Ala
        130 135 140
Asp Phe Ala Thr Thr Ile Trp Gln Gln Met Glu Glu Leu Gly Met Ala
145 150 155 160
Pro Ala Leu Gln Pro Thr Gln Gly Ala Met Pro Ala Phe Ala Ser Ala
                                165 170 175
Phe Gln Arg Arg Ala Gly Gly Val Leu Val Ala Ser His Leu Gln Ser
                        180 185 190
Phe Leu Glu Val Ser Tyr Arg Val Leu Arg His Leu Ala Gln Pro
                195 200 205
<210> SEQ ID NO 177
<211> LENGTH: 716
<212> TYPE: RNA
<213> ORGANISM: Homo sapiens
<400> SEQUENCE: 177
gggaaauaag agagaaaaga agaguaagaa gaaauauaag agccaccaug aacuuucucu 60
ugucaugggu gcacuggagc cuugcgcugc ugcuguaucu ucaucacgcu aaguggagcc 120
aggccgcacc cauggcggag gguggcggac agaaucacca cgaaguaguc aaauucaugg 180
acguguacca gaggucguau ugccauccga uugaaacucu uguggauauc uuucaagaau 240
accccgauga aaucgaguac auuuucaaac cgucgugugu cccucucaug aggugcgggg 300
gaugcugcaa ugaugaaggg uuggagugug uccccacgga ggagucgaau aucacaaugc 360
aaaucaugcg caucaaacca caucaggguc agcauauugg agagaugucc uuucuccagc 420
acaacaaaug ugaguguaga ccgaagaagg accgagcccg acaggaaaac ccaugcggac 480
cgugcuccga gcggcgcaaa cacuuguucg uacaagaccc ccagacaugc aagugcucau 540
guaagaauac cgauucgcgg uguaaggcga gacagcugga auugaacgag cgcacgugua 600
ggugcgacaa gccuagacgg ugagcugccu ucugcggggc uugccuucug gccaugcccu 660
ucuucucucc cuugcaccug uaccucuugg ucuuugaaua aagccugagu aggaag 716
<210> SEQ ID NO 178
<211> LENGTH: 4
<212> TYPE: PRT
<213> ORGANISM: Homo sapiens
<400> SEQUENCE: 178
Leu Val Pro Arg
1
<210> SEQ ID NO 179
<211> LENGTH: 4
<212> TYPE: PRT
<213> ORGANISM: Homo sapiens
<400> SEQUENCE: 179
Ile Glu Gly Arg
1
<210> SEQ ID NO 180
<211> LENGTH: 4
<212> TYPE: PRT
<213> ORGANISM: Homo sapiens
<400> SEQUENCE: 180
Ile Asp Gly Arg
1
<210> SEQ ID NO 181
<211> LENGTH: 4
<212> TYPE: PRT
<213> ORGANISM: Homo sapiens
<400> SEQUENCE: 181
Ala Glu Gly Arg
1
```

1. A method of treating a mammalian subject in need thereof comprising administering an mRNA encoding a polypeptide of interest.

2. The method of claim 1, wherein the mammalian subject is suffering from or is at risk of developing an acute or life-threatening disease or condition.

3. The method of claim 2, wherein the mammalian subject is suffering from a traumatic injury.

4. The method of claim 2, wherein the polypeptide of interest accelerates wound healing.

5. The method of claim 1, wherein the mammalian subject is suffering from a bacterial infection and wherein the polypeptide of interest is an anti-microbial peptide (AMP).

6. The method of claim 5, wherein the polypeptide of interest is an anti-viral.

7. The method of claim 1, wherein the polypeptide of interest is a cytokine.

8. The method of claim 7, wherein the mRNA is formulated and wherein the formulation is selected from the group consisting of lipid nanoparticle, polymer, hydrogel and surgical sealant.

9. The method of claim 8, wherein the formulated mRNA is administered to the mammalian subject by a route selected from the group consisting of transdermal, epicutaneous, intradermal, subcutaneous, intravenous, intramuscular, transdermal, topical, and systemic.

10. The method of claim 9, wherein the formulated mRNA is administered transdermally to the mammalian subject.

11. The method of claim 9, wherein the formulated mRNA is administered topically to the mammalian subject.

12. The method of claim 8, wherein the formulated mRNA is administered to the mammalian subject using bandages or dressings comprising the formulated mRNA.

13. The method of claim 1, wherein the polypeptide of interest is a protein expressed by macrophages.

14. The method of claim 13, wherein the mRNA is formulated and wherein the formulation is selected from the group consisting of lipid nanoparticle, polymer, hydrogel and surgical sealant.

15. The method of claim 14, wherein the formulated mRNA is administered to the mammalian subject by a route selected from the group consisting of transdermal, epicutaneous, intradermal, subcutaneous, intravenous, intramuscular, transdermal, topical, and systemic.

16. The method of claim 14, wherein the formulated mRNA is administered to the mammalian subject using bandages or dressings comprising the formulated mRNA.

17. The method of claim 1, wherein the polypeptide of interest is an angiogenic growth factor.

18. The method of claim 17, wherein the mRNA is formulated and wherein the formulation is selected from the group consisting of lipid nanoparticle, polymer, hydrogel and surgical sealant.

19. The method of claim 18, wherein the formulated mRNA is administered to the mammalian subject by a route selected from the group consisting of transdermal, epicutaneous, intradermal, subcutaneous, intravenous, intramuscular, transdermal, topical, and systemic.

20. The method of claim 18, wherein the formulated mRNA is administered to the mammalian subject using bandages or dressings comprising the formulated mRNA.
